(19) United States
(12) Patent Application Publication
(10) Pub. No.: US 2025/0140406 A1
(43) Pub. Date: May 1, 2025

(54) CLASSIFYING TUMORS AND PREDICTING RESPONSIVENESS

(71) Applicant: Oncocyte Corporation, Irvine, CA (US)

(72) Inventors: Robert Scott Seitz, Hampton Cove, AL (US); Brian Z. Ring, Foster City, CA (US); Catherine Cronister, Nashville, TN (US); David Hout, Franklin, TN (US); Tyler Jon Nielsen, Brentwood, TN (US); Brock Lloyd Schweitzer, Nashville, TN (US); Douglas T. Ross, Burlingame, CA (US)

(21) Appl. No.: 18/835,307

(22) PCT Filed: Feb. 1, 2023
(86) PCT No.: PCT/US2023/012158

§ 371 Date: Aug. 1, 2024

Related U.S. Application Data
(60) Provisional application No. 63/305,944, filed on Feb. 2, 2022.

(51) Int. Cl. G16H 50/20; G16B 20/00; G16B 25/10; G16B 40/20

(52) U.S. Cl. CPC G16H 50/20; G16B 20/00; G16B 25/10; G16B 40/20

(57) ABSTRACT

Cancer is the second leading cause of death in the United States. Increasingly, immune modulating therapies, such as therapy with immune checkpoint inhibitors (ICI) are being explored as promising potential therapies for many cancers. Presented herein are systems and methods for prediction, and especially automated prediction, of subject response to cancer therapies. Also presented herein are methods for selection of cancer therapies based upon predicted subject response and/or technologies for administering cancer therapies to appropriate subjects.

### CROSS-REFERENCE TO RELATED APPLICATIONS

This application claims priority to U.S. Provisional Application No. 63/305,944, filed Feb. 2, 2022, the entirety of which is incorporated herein by reference.

### BACKGROUND

Cancer is the second leading cause of death in the United States. Increasingly, immune modulating therapies, such as therapy with immune checkpoint inhibitors (ICI) are being explored as promising potential therapies for many cancers.

### SUMMARY

The present disclosure provides technologies for determining likelihood of patient responsiveness to certain therapies (e.g., for stratifying patient populations), and for treatment of cancer by administering such therapy to responsive patients and/or populations (and/or withholding such therapy and/or administering alternative therapy to non-responsive patients and/or populations), as defined herein. In particular, the present disclosure provides technologies for determining likelihood of patient responsiveness to immunomodulation therapy.

Without wishing to be bound by any particular theory, the present disclosure provides an insight that effective biomarkers for responsiveness to relevant therapy (e.g., immunomodulation therapy, and particularly ICI therapy) may be those that capture aspects of immunosurveillance, immunosuppression, and immune evasion as a tumor transitions from a proliferative to a metastatic state. Alternatively or additionally, the present disclosure provides an insight that effective biomarkers for responsiveness to immunomodulation therapy may asses one or more features of an immunological state of the tumor microenvironment (TME).

The present disclosure demonstrates, among other things, that assessment of a mesenchymal (M) gene expression signature, a mesenchymal stem-like (MSL) gene expression signature and an immunomodulatory (IM) gene expression signature can together provide an immuno-oncology score (an IO score) that is an effective biomarker for responsiveness to certain therapies (e.g. immunomodulation therapy, and particularly ICI therapy). In some embodiments, mesenchymal (M) gene expression signature, mesenchymal stem-like (MSL) gene expression signature and immunomodulatory (IM) gene expression signature are assessed through examination of a set of genes provided herein. In some embodiments, mesenchymal (M) gene expression signature, mesenchymal stem-like (MSL) gene expression signature and immunomodulatory (IM) gene expression signature are assessed through examination of genes determined through use of a gene expression algorithm.

In some embodiments, the present disclosure provides technologies for monitoring therapy administered to a cancer patient through assessment of an IO score over time. Alternatively or additionally, the present disclosure provides methods of selecting and/or adjusting therapies administered to a cancer patient through assessment of an IO score at multiple time points. In some embodiments, the present disclosure provides methods for selectively administering one or more therapies to a cancer patient determined to have an IO score meeting a certain threshold value.

Without wishing to be bound by a particular theory, the present disclosure provides an insight that assessment of an IO score can inform selection of a particular therapy (e.g. immunomodulation therapy, and particularly ICI therapy) for administration to a patient with a malignancy or potential malignancy. In some embodiments, the present disclosure provides an insight that assessment of an IO score can inform selection of a combination of one or more therapies, either in tandem or in sequence (e.g. comprising one or more immunomodulation therapies).

The present disclosure demonstrates, among other things, development of a tumor classifier effective to distinguish between responsiveness and non-responsiveness to immunomodulation therapy. In some embodiments, the present disclosure provides an insight that a tumor classifier can be trained for use in multiple different tumor types.

Alternatively or additionally, the present disclosure permits assessment of association (e.g., correlation) with classified IM, M, and/or MSL features. In some embodiments, the present disclosure permits identification and/or characterization of other parameters (e.g., RNA levels, gene expression, gene mutation, protein expression, protein modification, epigenetic modification, etc.) for association. In some embodiments, such associated features may comprise biomarkers that may be detected (e.g., measurement of presence and/or or levels). In some embodiments, such associated features may comprise a particular form (e.g., variant form (e.g., presence of a particular allele or mutation), modified form (e.g., epigenetic modification of a gene or gene associated sequence, phosphorylation or glycosylation of a protein, etc.), a particular one of known forms (e.g., splicing forms, allelelic forms, etc.), etc.) of one or more genes or gene products. In some embodiments, technologies provided herein permit assessment of association with IM, M, and/or MSL features, which can reveal presence and/or development of biological event(s) that recommend particular therapy be used in addition or as an alternative to immunomodulation therapy.

In some embodiments, the present disclosure provides a method of characterizing a potential cancer therapy by determining that said therapy directly or indirectly correlates with IM, M, and/or MSL features. In some embodiments, the present disclosure provides a method comprising a step of detecting in a subject who is a candidate for receiving a particular therapy a biomarker established to correlate with responsiveness or non-responsiveness to the therapy.

In some embodiments, the present disclosure provides a method of treating a subject in whom a biomarker has been detected, the method comprising steps of administering immunomodulation therapy or therapy that sensitizes to immunomodulation therapy if the therapy has been correlated with IM status and administering alternative therapy if the biomarker has been correlated with M or MSL subtype.

In some embodiments, the present disclosure provides a method of treating a subject in whom a biomarker has been detected, the method comprising steps of administering therapy that has been correlated with IM status if the biomarker has also been so correlated and administering therapy that has been correlated with M or MSL subtype if the therapy has also been so correlated.

In some embodiments, mesenchymal (M) gene expression signature, mesenchymal stem-like (MSL) gene expression signature and/or immunomodulatory (IM) gene expression signatures as provided here, and/or models or representations of tumor subtype and/or are used to establish and/or characterize (e.g., validate) biomarkers of tumor subtype or status (i.e., of IM, M, or MSL character), and/or of responsiveness to particular therapy, for example by demonstrating correlation with a provided gene expression signature and/or with a result (e.g., a heat map) of its application to tissue analysis.

Still further, by demonstrating effectiveness of provided technologies at classifying tumor subtype, status and/or responsiveness, the present disclosure provides technologies that permit investigation and/or interpretation of data such as clinical and/or cell line data, including relevant to development of resistance to one or more particular therapies (e.g., ICI therapy) and/or emergence of additional targets for therapy. Thus, in some embodiments, the present disclosure provides technologies for identifying and/or characterizing therapeutic targets, for selecting, administering and/or adjusting therapeutic regimens (e.g., to address or anticipate developing resistance and/or emerging target(s) in a particular subject or set of subjects.

Advantages of certain embodiments of provided technologies include that such assessment may be of data inputs from any of a variety of platforms; as documented herein, strategies provided by the present disclosure can provide an effective IO score biomarker independent of data input source.

### BRIEF DESCRIPTION OF THE DRAWINGS

FIG. 1: Common Immune Checkpoint Pathways and FDA-Approved ICIs.

Figure adapted from Hui et al., “Immune checkpoint inhibitors” J. Cell Biol. 218, 2019, incorporated herein by reference in its entirety. Artwork by Neil Smith (nel@neilsmithillustration.co.uk).

FIG. 2: Schematic of chimeric antigen receptor (CAR) structure, adapted from Feins et al et al., “An introduction to chimeric antigen receptor (CAR) T-cell immunotherapy for human cancer”, Am J Hematol. 94, 2019, incorporated herein by reference in its entirety.

FIG. 3: Major types of neoantigen vaccines, adapted from Peng et al., “Neoantigen vaccine: an emerging tumor immunotherapy”, Mol. Cancer, 18, 2019, incorporated herein by reference in its entirety.

FIG. 4: Mechanisms of Rescue of CAR T cell Exhaustion with Checkpoint Blockade, adapted from Grosser et al., “Combination Immunotherapy with CAR T Cells and Checkpoint Blockade for the Treatment of Solid Tumors”, Cancer Cell, 36, 2019, incorporated herein by reference in its entirety.

FIG. 5: Pathways interfering with PD-1 signaling, adapted from Langdon et al., “Combination of dual mTORC1/2 inhibition and immune-checkpoint blockade potentiates anti-tumour immunity”, Oncoimmunology, 7, 2018, incorporated herein by reference in its entirety.

FIG. 6: Gene selection process for building the 27-gene immuno-oncology algorithm. Gene set resulted from data set normalization, batch correction, gene set enrichment analysis, and elastic net modeling.

FIG. 7: Overview of IO score as a measure of the TME state.

FIG. 8: Mapping of IO score against gene signatures for bladder cancer data

FIG. 9: Association of IO scoring with gene signature classifications

FIG. 10: Placement of the 27 IO scores relative to the TME and identification of pathways associated with certain metagenes

FIG. 11: Confirmation of IO scoring threshold accuracy

FIG. 12: IO scoring as predictor of overall survival rates for bladder cancer ICI therapy trial.

FIG. 13: Mapping of gene expression and compound sensitivity in relation to subtypes with training set of cell lines.

FIG. 14: Mapping of gene expression and compound sensitivity in relation to subtypes with test set of cell lines.

FIG. 15: Correlation of immune signatures (red is positive correlation, blue is negative correlation).

FIG. 16: Mapping of gene expression and immune signatures in relation to subtypes for lung adenocarcinoma training data set.

FIG. 17: Mapping of gene expression and immune signatures in relation to subtypes for lung squamous cell training data set.

FIG. 18: Mapping of gene expression and immune signatures in relation to subtypes for lung adenocarcinoma test data set.

FIG. 19: Mapping of gene expression and immune signatures in relation to subtypes for lung squamous cell carcinoma test data set.

FIG. 20A: Correlation of miRNA expression to DTIO binary call for pre-miRNA validation set; FIG. 20B: pre-miRNA test set; FIG. 20C: mature miRNA validation set; and FIG. 20D: mature miRNA test set.

FIG. 21: Representation of sets of co-expressed genes across fifteen different tumor types and resulting immune infiltrate signatures.

FIG. 22: Comparison of immune network signatures to ImmGen signatures.

FIG. 23: Comparison of immune network signatures to xCell signatures.

FIG. 24A: Mapping of gene expression and immune signatures in relation to subtypes for breast test data set; FIG. 24B: for lung adenocarcinoma training data set;

FIG. 24C: for lung squamous cell carcinoma training data set; FIG. 24D: for colon carcinoma test data set; and FIG. 24E: for bladder carcinoma test data set;

FIG. 25A: Mapping of gene expression and immune signatures in relation to subtypes for TCGA training set; FIG. 25B: for TCGA test set; and FIG. 25C: for both training and test set.

FIG. 26A: Distribution of immune network signatures in relation to IM, M, and MSL subtypes for lymphoid training set; FIG. 26B: for myeloid training set; FIG. 26C: for lymphoid test set; FIG. 26D: for myeloid test set; FIG. 26E: for lympohid training and test sets; FIG. 26F: for myeloid training and test sets.

FIG. 27A: Kaplan-Meier plots of patient survival for patients for network B-cell signature for IMVIgor cohort; FIG. 26B: for network non-B lymphoid signature for IMVIgor cohort; FIG. 27C: for network B-cell signature for UNC bladder samples;

FIG. 27D: for network non-B lymphoid signature for UNC bladder samples.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” refers to the administration of a composition to a subject or system (e.g., to a cell, organ, tissue, organism, or relevant component or set of components thereof). Those of ordinary skill will appreciate that route of administration may vary depending, for example, on the subject or system to which the composition is being administered, the nature of the composition, the purpose of the administration, etc. For example, in certain embodiments, administration to an animal subject (e.g., to a human) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Agonist Therapy: The term “agonist therapy”, as used herein, refers to administration of an agonist that agonizes a particular target of interest to achieve a desired therapeutic effect. In some embodiments, agonist therapy involves administering a single dose of an agonist. In some embodiments, agonist therapy involves administering multiple doses of an agonist. In some embodiments, agonist therapy involves administering an agonist according to a dosing regimen known or expected to achieve the therapeutic effect, for example, because such result has been established to a designated degree of statistical confidence, e.g., through administration to a relevant population.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTERs; ankyrin repeat proteins or DARPINSR; Avimers®; DARTs; TCR-like antibodies; Adnectins®; Affilins®; Trans-Bodies®; AffibodiesR; TrimerXR; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINS®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody component: as used herein, refers to a polypeptide element (that may be a complete polypeptide, or a portion of a larger polypeptide, such as for example a fusion polypeptide as described herein) that represents a portion of an antibody or antibody agent. In some embodiments, an antibody component includes one or more immunoglobulin structural features. In some embodiments, an antibody component specifically binds to an antigen. Typically, an antibody component is a polypeptide whose amino acid sequence includes elements characteristic of an antibody-binding region (e.g., an antibody light chain variable region or one or more complementarity determining regions (“CDRs”) thereof, or an antibody heavy chain or variable region or one more CDRs thereof, optionally in presence of one or more framework regions). In some embodiments, an antibody component is or comprises a full-length antibody. In some embodiments, the term “antibody component” encompasses any protein having a binding domain, which is homologous or largely homologous to an immunoglobulin-binding domain. In particular embodiments, an included “antibody component” encompasses polypeptides having a binding domain that shows at least 99% identity with an immunoglobulin binding domain. In some embodiments, an included “antibody component” is any polypeptide having a binding domain that shows at least 70%, 75%, 80%, 85%, 90%, 95% or 98% identity with an immunoglobulin binding domain, for example a reference immunoglobulin binding domain. An included “antibody component” may have an amino acid sequence identical to that of an antibody (or a portion thereof, e.g., an antigen-binding portion thereof) that is found in a natural source. An antibody component may be monospecific, bi-specific, or multi-specific. An antibody component may include structural elements characteristic of any immunoglobulin class, including any of the human classes: IgG, IgM, IgA, IgD, and IgE. It has been shown that the antigen-binding function of an antibody can be performed by fragments of a full-length antibody. Such antibody embodiments may also be bispecific, dual specific, or multi-specific formats specifically binding to two or more different antigens. Examples of binding fragments encompassed within the term “antigen-binding portion” of an antibody include (i) a Fab fragment, a monovalent fragment consisting of the V_{H}, V_{L}, C_{H}1 and C_{L} domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{H} and V_{L} domains of a single arm of an antibody, (v) a dAb fragment (Ward et al., (1989) Nature 341:544-546), which comprises a single variable domain; and (vi) an isolated complementarity determining region (CDR). Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In some embodiments, an “antibody component”, as described herein, is or comprises such a single chain antibody. In some embodiments, an “antibody component” is or comprises a diabody. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., (1993) Proc. Natl. Acad. Sci. USA 90:6444-6448; Poljak, R. J., (1994) Structure 2(12): 1121-1123). Such antibody binding portions are known in the art (Kontermann and Dubel eds., Antibody Engineering (2001) Springer-Verlag. New York. 790 pp. (ISBN 3-540-41354-5). In some embodiments, an antibody component is or comprises a single chain “linear antibody” comprising a pair of tandem Fv segments (V_{H}-CH1-V_{H}-CH1) which, together with complementary light chain polypeptides, form a pair of antigen binding regions (Zapata et al., (1995) Protein Eng. 8(10): 1057-1062; and U.S. Pat. No. 5,641,870). In some embodiments, an antibody component may have structural elements characteristic of chimeric or humanized antibodies. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some embodiments, an antibody component may have structural elements characteristic of a human antibody.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to a to an entity whose presence, level, or form, correlates with a particular biological event or state of interest, so that it is considered to be a “marker” of that event or state. To give but a few examples, in some embodiments, a biomarker may be or comprises a marker for a particular disease state, or for likelihood that a particular disease, disorder or condition may develop. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be an entity of any chemical class. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is a gene. In some embodiments, a biomarker is a gene associated with a particular cell type. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc.). In some embodiments, a biomarker is a particular form (e.g., variant form (e.g., presence of a particular allele or mutation), modified form (e.g., epigenetic modification of a gene or gene associated sequence, phosphorylation or glycosylation of a protein, etc.), a particular one of known forms (e.g., splicing forms, allelelic forms, etc.), etc.) of one or more genes or gene products.

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.

Characteristic sequence: A “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Combination Therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: A “composition” or a “pharmaceutical composition” according to this invention refers to the combination of two or more agents as described herein for co-administration or administration as part of the same regimen. It is not required in all embodiments that the combination of agents result in physical admixture, that is, administration as separate co-agents each of the components of the composition is possible; however many patients or practitioners in the field may find it advantageous to prepare a composition that is an admixture of two or more of the ingredients in a pharmaceutically acceptable carrier, diluent, or excipient, making it possible to administer the component ingredients of the combination at the same time.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Certain methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage Form: As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Diagnostic information: As used herein, “diagnostic information” or “information for use in diagnosis” is information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosing Regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Effector function: as used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: as used herein refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Engineered: Those of ordinary skill in the art, reading the present disclosure, will appreciate that the term “engineered”, as used herein, refers to an aspect of having been manipulated and altered by the hand of man. In particular, the term “engineered cell” refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Genome: As used herein, the term “genome” refers to the total genetic information carried by an individual organism or cell, represented by the complete DNA sequences of its chromosomes.

Genome Profile: As used herein, the term “genome profile” refers to a representative subset of the total information contained within a genome. Typically, a genome profile contains genotypes at a particular set of polymorphic loci. In some embodiments, a genome profile may correlate with a particular feature, trait, or set thereof characteristic of, for example, a particular animal, line, breed, or crossbreed population.

Host: The term “host” is used herein to refer to a system (e.g., a cell, organism, etc) in which a polypeptide of interest is present. In some embodiments, a host is a system that is susceptible to infection with a particular infectious agent. In some embodiments, a host is a system that expresses a particular polypeptide of interest.

Host cell: as used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Inducible Effector Cell Surface Marker: As used herein, the term “inducible effector cell surface marker” refers to an entity, that typically is or includes at least one polypeptide, expressed on the surface of immune effector cells, including without limitation natural killer (NK) cells, which expression is induced or significantly upregulated during activation of the effector cells. In some embodiments, increased surface expression involves increased localization of the marker on the cell surface (e.g., relative to in the cytoplasm or in secreted form, etc). Alternatively or additionally, in some embodiments, increased surface expression involves increased production of the marker by the cell. In some embodiments, increased surface expression of a particular inducible effector cell surface marker correlates with and/or participates in increased activity by the effector cell (e.g., increased antibody-mediated cellular cytotoxicity [ADCC]). In some embodiments, an inducible effector cell surface marker is selected from a group consisting of a member of the TNFR family, a member of the CD28 family, a cell adhesion molecule, a vascular adhesion molecule, a G protein regulator, an immune cell activating protein, a recruiting chemokine/cytokine, a receptor for a recruiting chemokine/cytokine, an ectoenzyme, a member of the immunoglobulin superfamily, a lysosomal associated membrane protein. Certain exemplary inducible cell surface markers include, without limitation, CD38, CD137, OX40, GITR, CD30, ICOS, etc. In some particular embodiments, the term refers to any of the above-mentioned inducible cell surface markers other than CD38.

Inhibitory agent: As used herein, the term “inhibitory agent” refers to an entity, condition, or event whose presence, level, or degree correlates with decreased level or activity of a target). In some embodiments, an inhibitory agent may be act directly (in which case it exerts its influence directly upon its target, for example by binding to the target); in some embodiments, an inhibitory agent may act indirectly (in which case it exerts its influence by interacting with and/or otherwise altering a regulator of the target, so that level and/or activity of the target is reduced). In some embodiments, an inhibitory agent is one whose presence or level correlates with a target level or activity that is reduced relative to a particular reference level or activity (e.g., that observed under appropriate reference conditions, such as presence of a known inhibitory agent, or absence of the inhibitory agent in question, etc.).

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre and post natal forms. In some embodiments, a patient is suffering from or susceptible to one or more disorders or conditions. In some embodiments, a patient displays one or more symptoms of a disorder or condition. In some embodiments, a patient has been diagnosed with one or more disorders or conditions

Pharmaceutically Acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutical Composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. Prevention may be considered complete when onset of a disease, disorder or condition has been delayed for a predefined period of time.

Prognostic and predictive information: As used herein, the terms “prognostic information” and “predictive information” are used to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Receptor tyrosine kinase: The term “receptor tyrosine kinase”, as used herein, refers to any members of the protein family of receptor tyrosine kinases (RTK), which includes but is not limited to sub-families such as Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2, EphB3, EphB4, and EphB6).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Refractory: The term “refractory” as used herein, refers to any subject or condition that does not respond with an expected clinical efficacy following the administration of provided compositions as normally observed by practicing medical personnel.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3): 205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Sample: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Solid Tumor: As used herein, the term “solid tumor” refers to an abnormal mass of tissue that usually does not contain cysts or liquid areas. Solid tumors may be benign or malignant. Different types of solid tumors are named for the type of cells that form them. Examples of solid tumors are sarcomas, carcinomas, lymphomas, mesothelioma, neuroblastoma, retinoblastoma, etc.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Stage of cancer: As used herein, the term “stage of cancer” refers to a qualitative or quantitative assessment of the level of advancement of a cancer. In some embodiments, criteria used to determine the stage of a cancer may include, but are not limited to, one or more of where the cancer is located in a body, tumor size, whether the cancer has spread to lymph nodes, whether the cancer has spread to one or more different parts of the body, etc. In some embodiments, cancer may be staged using the so-called TNM System, according to which T refers to the size and extent of the main tumor, usually called the primary tumor; N refers to the the number of nearby lymph nodes that have cancer; and M refers to whether the cancer has metastasized. In some embodiments, a cancer may be referred to as Stage 0 (abnormal cells are present but have not spread to nearby tissue, also called carcinoma in situ, or CIS; CIS is not cancer, but it may become cancer), Stage I-III (cancer is present; the higher the number, the larger the tumor and the more it has spread into nearby tissues), or Stage IV (the cancer has spread to distant parts of the body). In some embodiments, a cancer may be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to figure out the stage).

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, terms “individual” or “patient” are used and are intended to be interchangeable with “subject”. In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, and/or condition displays one or more symptoms of a disease, disorder, and/or condition and/or has been diagnosed with the disease, disorder, or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Surrogate Marker: The term “surrogate marker”, as used herein, refers to an entity whose presence, level, or form, may act as a proxy for presence, level, or form of another entity (e.g., a biomarker) of interest. Typically, a surrogate marker may be easier to detect or analyze (e.g., quantify) than is the entity of interest. To give but one example, in some embodiments, where the entity of interest is a protein, an expressed nucleic acid (e.g., mRNA) encoding the protein may sometimes be utilized as a surrogate marker for the protein (or its level). To give another example, in some embodiments, where the entity of interest is an enzyme, a product of the enzyme's activity may sometimes be utilized as a surrogate marker for the enzyme (or its activity level). To give one more example, in some embodiments, where the entity of interest is a small molecule, a metabolite of the small molecule may sometimes be used as a surrogate marker for the small molecule.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition).

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic Regimen: A “therapeutic regimen”, as that term is used herein, refers to a dosing regimen whose administration across a relevant population is correlated with a desired or beneficial therapeutic outcome.

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in any of the pro-angiogenic markers discussed above, an increase in anti-angiogenic markers described herein, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### DETAILED DESCRIPTION OF CERTAIN EMBODIMENTS

### Cancer Subtype Classification

Molecular classification of cancer subtypes is becoming an increasingly important tool, both for understanding tumor development and progression, and for designing treatment plans for particular tumors and/or tumor subtypes. Indeed, potential new therapies are now commonly evaluated and/or approved based on presence of a particular molecular signature established to correlate with responsiveness to the relevant therapy (and/or absence of a molecular signature established to negatively correlate with such responsiveness), for example as may be assessed via basket trials, and/or based on molecular subtyping of a relevant disease, disorder or condition, for example as may be assessed via umbrella trials. See, for example, Park et al. “An Overview of Precision Oncology Basket and Umbrella Trials for Clinicians” CA Cancer J Clin 70:125, March/April 2020, incorporated herein by reference in its entirety.

Work by Lehmann et al. (See Lehman et al. “Identification of human triple-negative breast cancer subtypes and preclinical models for selection of targeted therapies” J Clin Invest, 121(7), 2011, incorporated herein by reference in its entirety) has demonstrated that triple negative breast cancer (TNBC) tumors can be classified into subtypes through analysis of gene expression signatures. Lehmann et al. determined gene expression profiles for annotated genes within publicly available TNBC samples and performed centroid-based cluster analysis based upon the 20% of genes with the highest and lowest expression levels in at least 50% of the samples (2188 genes total). Clusters were categorized based upon features of differentially expressed genes, leading to identification of six different subtypes, specifically: basal-like 1 (BL1), basal-like 2 (BL2), immunomodulatory (IM), mesenchymal (M), mesenchymal stem-like MSL), and luminal androgen receptor (LAR). It was found that there was significant heterogeneity within TNBC tumors. Furthermore, Lehmann et al reported that certain cell lines representative of different subtypes showed differential response to certain therapies. Table 1 below summarizes the specific findings reported in Lehmann et al.:

TABLE 1
Tumor subtypes from Lehman et al.

| | | Treatments to which |
|---|---|---|
| | Highly | Representative Cell Lines |
| Tumor Subtype | Expressed Genes | Respond |
| BL1 and BL2 | Cell cycle genes | Cisplatin |
| | DNA repair genes | |
| IM | Genes involved in immune | NA |
| | cell processes | |
| M and MSL | Genes involved in epithelial- | NVP-BEX235 |
| | mesenchymal transition | (a PI3K/mTOR inhibitor) |
| | Growth factor pathway | Dasantinib |
| | genes | (an abl/src inhibitor) |
| LAR | Androgen receptor signaling | Bicalutimide |
| | genes | (an AR antagonist) |

Lehmann et al. concluded that gene expression analyses can be useful to define distinct subtypes of TNBC, and further proposed that such analyses “may provide biomarkers that can be used for patient selection in the design of clinical trials for TNBC and/or as potential markers for response to treatment”; Lehmann et al also recommended that further such analyses, together with RNAi loss-of-function screens be performed in order to “identify new components of the “driver” signaling pathways in each of these subtypes that can be targeted in future drug discovery efforts for TNBC″. See last paragraph of “Conclusion” section of, Lehman et al. “Identification of human triple-negative breast cancer subtypes and preclinical models for selection of targeted therapies” J Clin Invest, 121(7), 2011.

Ring et al. (See, Ring et al. “Generation of an algorithm based on minimal gene sets to clinically subtype triple negative breast cancer patients” BMC Cancer, 16, February 2016, incorporated herein by reference in its entirety) independently analyzed the same gene expression datasets utilized by Lehman et al., to identify genes enriched in different TNBC subtypes, and then further performed shrunken centroid analysis and elastic-net regularized linear modeling to define a set of genes whose expression could be analyzed to classify TNBC samples into the defined subtypes. Specifically, Ring et al. used linear regression, targeted maximum likelihood estimation, random forest, and elastic-net regularized linear models to create subclassifying models, with each subclass (subtype) being defined by an individual model (See, Subramanian et al., “Gene set enrichment analysis: a knowledge-based approach for interpreting genome-wide expression profiles”, PNAS, 102, 2015; see also, Friedman et al., “Regularization Paths for Generalized Linear Models via Coordinate Descent”, J Stat Softw, 33, 2010; see also, Hajian-Tilaki et al., “Receiver Operating Characteristic (ROC) Curve Analysis for Medical Diagnostic Test Evaluation”, 4, 2013, each of which is incorporated herein by reference in its entirety). Genes found to contribute to the individual subtype models were combined to create a 101-gene centroid model for TNBC subtype classification. This Ring et al. model represented a significant simplification, relative to the Lehmann et al. model, which relied on expression information for 2188 genes.

Furthermore, Ring et al. observed that gene expression Lehmann et al. had associated with the IM tumor subtype in fact was not reflective of tumor-cell expression at all but likely reflected presence of tumor infiltrating lymphocytes (TIL) in relevant tumor samples. Exclusion of IM gene signatures led to loss of information for samples, so the IM subtype was removed and cases initially assigned to this classification were analyzed separately. As a result, Ring et al. reduced the TNBC classes to five subtypes: BL1, BL2, LAR, M, and MSL; each of which could be reliably identified through use of the reduced 101-gene panel.

Ring et al. also reported preliminary evidence that subtype classification using its 101 gene model could be useful for predicting patient outcomes for certain therapies. For example, Ring et al reported that BLI and BL2 TNBC subtypes, as defined using its 101 gene model, differ in their pathological response to mitotic inhibitors; BL1 subtype tumors tended to have a better response rate. As other classification approaches (including both the Lehmann et al. 2188-gene model and traditional pathological assessments) had similarly noted better prognosis for chemotherapy with BLI subtype tumors relative to BL2 subtype, this finding was considered to provide initial validation that the Ring et al. 101 gene model represented important progress toward development of predictive assessment tool; however Ring et al itself notes both that further clinical validation of predictive success would be required to establish a medically useful tool and, furthermore, that reduced gene sets able “to individually classify each subtype” still needed to be developed.

It is worth noting that, in subsequent work, Lehmann et al. observed that tumors assigned by the 2188 gene model to a primary M classification did not have a secondary correlation to the IM subtype also defined by that model. See, Lehmann et al. “Refinement of Triple-Negative Breast Cancer Molecular Subtypes: Implications for Neoadjuvant Chemotherapy Selection”, 11, June 2016, incorporated herein by reference. In fact, M subtype tumors demonstrated a strong negative correlation with the gene expression features of the IM subtype. As noted above, Ring et al subsequently established that the IM signature observed by Lehmann et al was not in fact a tumor subtype, but rather represented presence of TIL in the samples. This observation that the IM signature represented gene expression by TIL was confirmed by Grigoriadis et al., who furthermore noted that each of the five actual tumor subtypes could be further classified by either a positive or negative IM gene signature. See, Grigoriadis et al. “Mesenchymal subtype negatively associates with the presence of immune infiltrates within a triple negative breast cancer classifier”, 2016 San Antonio Breast Cancer Symposium, December 2016, incorporated herein by reference in its entirety).

The present disclosure provides technologies for improved cancer subtype classification and, moreover, provides technologies for predicting tumor responsiveness to particular immunotherapies (e.g., to immune checkpoint inhibitor therapies).

Among other things, the present disclosure (1) provides technologies for establishing small gene sets (i.e., involving about 10 to about 50, or preferably about 10 to about 30 genes) whose expression patterns accurately subtype tumor samples; (2) provides an insight that consideration of including mesenchymal (M) subtype signature and also immunomodulatory (IM) status, and in certain embodiments including each of (a) M subtype, (b) mesenchymal-stem-like (MSL) subtype, and also (c) IM status, permits effective assessment of likely responsiveness to immunotherapies such as immune checkpoint inhibitor therapies; and (3) that assessment of IM status (as a positive predictor of responsiveness) vs M and/or MSL status (as a negative predictor of responsiveness) using the provided small gene set effectively determines likelihood of tumor responsiveness to immune checkpoint inhibitor therapy.

The present disclosure exemplifies provided technologies in the context of both triple negative breast and non-small cell lung cancer, and teaches its applicability across cancers (e.g., across solid tumors).

Among other things, the present disclosure solves certain problems associated with tumor subtyping and/or predicting such responsiveness. For example, in a study of gene signatures associated with tumor inflammation and epithelial-to-mesenchymal transition in lung cancer, Thompson et al. described “Disagreement [that] exists in the literature about the relationship of inflammatory genes to the mesenchymal phenotype”. See Thompson et al., “Gene signatures of tumor inflammation and epithelial-to-mesenchymal transition (EMT) predict responses to immune checkpoint blockade in lung cancer with high accuracy”, Lung Cancer, 139, 2020, incorporated herein by reference. Specifically, Thompson et al. noted that other researchers (Chae et al, “Epithelial mesenchymal transition (EMT) signature is inversely associated with T-cell infiltration in non-small cell lung cancer (NSCLC)”, Sci. Rep., 8, 2018) had “found that a more mesenchymal signature was associated with lower T cell gene expression in NSCLC” which they contrasted with their own data, which they described as “showing that tumors with higher inflammation scores had higher (more mesenchymal) EMT scores”, which they observed was “similar” to reports from yet others (Lou et al, “Epithelial-mesenchymal transition is associated with a distinct tumor microenvironment including elevation of inflammatory signals and multiple immune checkpoints in lung adenocarcinoma”, Clin. Cancer Res., 22, 2016, and Chen et al., “Metastasis is regulated via microRNA-200/ZEB1 axis control of tumour cell PD-L1 expression and intratumoral immunosuppression”, Nat. Commun., 5, 2014, each of which is incorporated herein by reference.

The present disclosure provides technologies that define small gene sets effective for tumor subtype classification, and furthermore for comparison of “M” and/or “MSL” vs “IM” status, while establishing benefit of a combined “positive”/“negative” assessment approach, considering both IM (positive) and M and/or MSL (negative) features, for determining tumor responsiveness to immunomodulation therapy such as immune checkpoint inhibitor therapy.

Among other things, the present disclosure provides technologies for assigning an immuno-oncology (IO) score to a tumor sample by assessing both the negative predicting features of the M subtype and the positive predicting features of the IM status through gene expression analysis of a small set (e.g., about 10 to about 50, or preferably about 10 to about 30) of genes. In some embodiments, the present disclosure provides technologies for assigning an IO score to a tumor sample by assessing both the negative predicting features of the MSL subtype and the positive predicting features of the IM status through gene expression analysis of a small set (e.g., about 10 to about 50, or preferably about 10 to about 30) of genes. In some embodiments, the present disclosure provides technologies for assigning an IO score to a tumor sample by assessing both the negative predicting features of the M and MSL subtype and the positive predicting features of the IM status through gene expression analysis of a small set (e.g., about 10 to about 50, or preferably about 10 to about 30) of genes. The present disclosure exemplifies effectiveness of provided strategies, including by development of a 27-gene panel established to be effective for tumor subtype classification and characterization of likely responsiveness (or resistance) as described herein.

Importantly, the present disclosure demonstrates that, unlike previous cancer subtyping and scoring methods, provided technologies can develop small gene sets (e.g., including about 10 to about 50, or even about 10 to about 30 genes) effective to classify tumor subtypes and furthermore to predict tumor responsiveness across different cancers. Indeed, literature reports have declared that “it is improbable to predict wide-ranging clinical benefits without using a wide set of biomarkers”. See, Fares et al. “Mechanisms of Resistance to Immune Checkpoint Blockade”, ACSO Educational Book, 39, 2019, incorporated herein by reference. The present disclosure demonstrates surprising success in this area of acknowledged challenge.

Without wishing to be bound by any particular theory, the present disclosure provides an insight that consideration of conditions of the tumor microenvironment may contribute to successful development of predictive models as described herein. For example, in some embodiments, the present disclosure teaches potentially excluding from gene sets utilized for assessment of tumor subtype and/or responsiveness to immunomodulation therapy (e.g., to immune checkpoint inhibitor therapy) as described herein genes, such as those that encode for the TGF-β family of proteins (e.g. TGFB1), that participate broadly in multiple cellular functions. In some embodiments, the present disclosure teaches that focus on more downstream genes and/or on genes involved in features of the tumor microenvironment.

Among other things, the present disclosure therefore provides a medically useful tool for classifying tumor samples and/or for predicting likely prognosis and/or predicting likely responsiveness of the tumor(s) to particular therapeutic modalities and/or treatment regimens, and specifically to immunomodulation therapy treatments such as immune checkpoint inhibitor therapy when appropriate or to therapies which act upon the tumor microenvironment to enhance immunogenicity and improve responsiveness to immunomodulation therapy treatments such as immune checkpoint inhibitor therapy when appropriate.

In some embodiments, the present disclosure provides kits for detecting expression of gene expression signatures in or from tumor samples, as well as technologies for selecting, monitoring, and/or adjusting therapies administered.

Alternatively or additionally, in some embodiments, the present disclosure provides technologies for developing small gene sets (e.g., including about 10 to about 50, or even about 10 to about 30 genes) and/or for establishing their effectiveness in classifying tumor samples and/or in predicting likely prognosis and/or responsiveness to particular therapeutic modalities and/or treatment regimens, and specifically to immunomodulation therapy treatments such as immune checkpoint inhibitor therapy.

### Immunomodulation Therapy

As noted herein, the present disclosure provides insights relating to responsiveness of particular tumors (i.e., patients) to particular therapy, and specifically to immunomodulation therapy. Without wishing to be bound by any particular theory, the present disclosure teaches that consideration of particular markers (e.g., those reflective of a mesenchymal and/or mesenchymal-like state, and/or those reflective of immunological activity within the tumor microenvironment) together can distinguish between and among tumors that (a) are in an immunologically “cold” state and are unlikely to respond to immunomodulation therapy; (b) are in an immunologically “hot” state and are likely to respond to immunomodulation therapy; and (c) are in an immunologically “poised” state, susceptible to transition to a “hot” state (e.g., by exposure to a particular treatment or therapy which may, in some embodiments, be or comprise immunomodulation therapy or may be or comprise other therapy, for example that may enhance immunogenicity for subsequent treatment by immunomodulation therapy).

Among other things, the present disclosure provides an insight that consideration of “intrinsic” vs “extrinsic” features characteristic of an immunological state can provide useful and valuable information, including as is relevant to therapy. For example, in some embodiments, assessment of immunological state can consider: (i) properties of a tumor itself; and/or (ii) properties of surrounding non-malignant, stromal tissue. In some embodiments, properties according to (i) may be referred to as intrinsic features and may correlate to the M or mesenchymal subtype. In some embodiments, properties according to (ii) may be referred to as extrinsic features and correlates to the MSL or mesenchymal-stem-like subtype. Without wishing to be bound by any particular theory, the present disclosure proposes, in some embodiments, that intrinsic and extrinsic features work together (e.g., synergistically with each other) to promote immune escape, e.g., where an immune system loses its capability adequately impede the growth of a tumor (See, Seliger, B. and C. Massa, Immune Therapy Resistance and Immune Escape of Tumors. Cancers, 2021. 13(3): p. 551; Xiong, J., H. Wang, and Q. Wang, Suppressive Myeloid Cells Shape the Tumor Immune Microenvironment. Advanced Biology, 2021. 5(3): p. 1900311; Xiao, Y. and D. Yu, Tumor microenvironment as a therapeutic target in cancer. Pharmacol Ther, 2021. 221: p. 107753, each of which is incorporated herein by reference in its entirety); in some embodiments, according to the present disclosure, assessment of such features can therefore inform likelihood that the relevant tumor will or will not be responsive to a particular therapy . . .

In some embodiments, intrinsic features of an immunologically “cold” state may include one or more of (i) an ability to evade recognition by the immune system (e.g., through mutations in tumor DNA); and (ii) having undergone epithelial to mesenchymal transition (See, Seliger, B. and C. Massa, Immune Therapy Resistance and Immune Escape of Tumors. Cancers, 2021. 13(3): p. 551; McGranahan, N., et al., Allele-Specific HLA Loss and Immune Escape in Lung Cancer Evolution. Cell, 2017. 171(6): p. 1259-1271.e11, each of which is incorporated herein by reference in its entirety).

In some embodiments, extrinsic features of an immunologically “cold” state may include one or more of (i) increased presence of immune-suppressive cells (e.g., certain types of CAFs, M2 macrophages, N2 neutrophils); (ii) increased vascularization of the tumor microenvironment (TME); and (iii) increased expression of one or more extracellular matrix genes (ECM) (See, Seliger, B. and C. Massa, Immune Therapy Resistance and Immune Escape of Tumors. Cancers, 2021. 13(3): p. 551; Desbois, M. and Y. Wang, Cancer-associated fibroblasts: Key players in shaping the tumor immune microenvironment. Immunological Reviews, 2021. 302(1): p. 241-258, each of which is incorporated herein by reference in its entirety).

In some embodiments, tumors classified as having high expression levels of genes with an M subtype (e.g., a tumor with M subtype) may secrete factors that recruit cancer-associated fibroblasts (CAFs) to the stroma. In some embodiments, CAFs may secrete factors such as fibroblast growth factor (FGF) and Wnt Family Member 3A (Wnt-3a) to promote proliferation in cancer cells and stromal derived factor-1 (CXCL12) to increase metastatic potential.

In some embodiments, immunosuppression in the stroma (e.g., in a tumor with MSL subtype) may limit types of immune cells targeting a tumor. In some embodiments, a tumor with M or MSL subtype may appear non-antigenic to one or more immune cells.

In some embodiments, the present disclosure provides technologies for administering (and/or monitoring and/or refraining from administering) certain therapies, e.g., an immunomodulatory therapy such as ICI therapy. Alternatively or additionally, in some embodiments, the present disclosure provides technologies for administering (and/or monitoring and/or refraining from administering) an immunomodulatory therapy such as T-cell therapy (e.g., CAR-T therapy) and/or vaccine therapy (e.g., neoantigen vaccination). Still further alternatively or additionally, in some embodiments, the present disclosure provides technologies for administering (and/or monitoring and/or refraining from administering) one or more combination therapies including, for example a combination of a non-immunomodulatory therapy (e.g., chemotherapy, radiation therapy, surgery, etc) with an immunomodulation therapy (e.g., ICI therapy, T cell therapy, vaccination, etc). Indeed, in some embodiments, treatment with another therapy may sensitize or otherwise enhance responsiveness of tumor to immunomodulation therapy, e.g., by enhancing the immunogenicity state of the tumor, as may in some embodiments be assessed, for example, as described herein.

### Immune Checkpoint Inhibitory Therapy

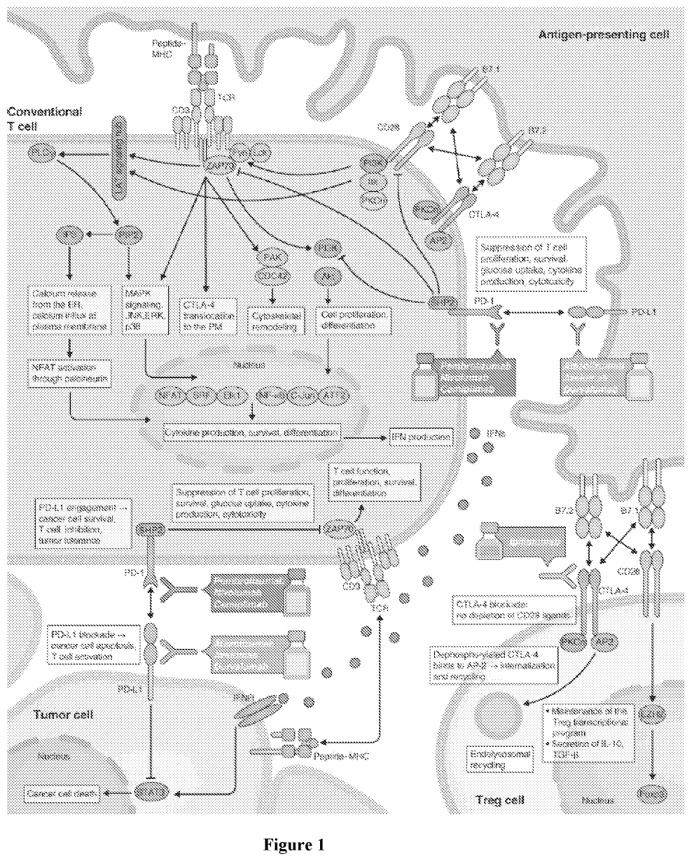

Recent research has shown that malignant cells can escape immunosurveillance through different mechanisms, including activation of immune checkpoint pathways that can suppress immune responses. T cells typically target tumor cells through two main mechanisms: 1) antigen-specific signals mediated by T cell receptors or 2) antigen-nonspecific signals through co-signaling receptors (see FIG. 1). Cellular expression of co-signaling receptors can either activate T-cell response (co-stimulatory receptors) or reduce T cell response (co-inhibitory receptors). See, for example, Chen et al. “Molecular mechanisms of T cell co-stimulation and co-inhibition” Nat. Rev. Immunol., 13, 2013, incorporated herein by reference in its entirety.

Tumor cells that express co-inhibitory receptors are able to “hide” as functional host tissue to evade immune recognition and attack. Inhibitory factors, e.g. antibodies, that bind to co-inhibitory immune checkpoints can interrupt these pathways and promote an immune response targeting tumor cells. These immune checkpoint inhibitors (ICIs) can target various immune checkpoints, including, for example, CTLA-4 (CD 152), PD-1, PD-L1, BTLA, VISTA, TIM-3, LAG3, CD47, and TIGIT, as well as their respective binding partners. ICIs can also target various co-stimulatory molecules, including, for example, CD137, OX40, and GITR. See, for example, Advani et al. “CD47 Blockage by Hu5F9-G4 and Rituximab in Non-Hodgkin's Lymphoma” N. Engl. J. Med., 379, 2018; Anderson et al., “Promotion of tissue inflammation by the immune receptor Tim-3 expressed on innate immune cells” Science, 318, 2007; Fourcade et al. “CD8(+) T cells specific for tumor antigens can be rendered dysfunctional by the tumor microenvironment through upregulation of the inhibitory receptors BTLA and PD-1” Cancer Res., 72, 2012; Gough et al. “Adjuvant therapy with agonistic antibodies to CD134 (OX40) increases local control after surgical or radiation therapy of cancer in mice” J. Immunother., 33, 2010; Hernandez-Chacon et al., “Costimulation through the CD137/4-1BB pathway protects human melanoma tumor-infiltrating lymphocytes from activation-induced cell death and enhances antitumor effector function” J. Immunother., 34, 2011; Lines et al. “VISTA is an immune checkpoint molecule for human T cells” Cancer Res., 74, 2014; Ngiow et al. “Anti-TIM3 antibody promoters T cell IFN-gamma-mediated antitumor immunity and suppresses established tumors” Cancer Res., 71, 2011; Schaer et al. “Anti-GITR antibodies-potential clinical applications for tumor immunotherapy” Curr. Opin. Investig. Drugs, 11, 2010; Wang et al. “VISTA, a novel mouse Ig superfamily ligand that negatively regulates T cell responses” J. Exp. Med., 208, 2011; Watanabe et al. “BTLA is a lymphocyte inhibitory receptor with similarities to CTLA-4 and PD-1” Nat. Immunol., 4, 2003; Woo et al. “Immune inhibitory molecules LAG-3 and PD-1 synergistically regulate T-cell function to promote tumoral immune escape” Cancer Res., 72, 2012; Vaddepally et al. “Review of Indications of FDA-Approved Immune Checkpoint Inhibitors per NCCN Guidelines with the Level of Evidence” Cancers, 12, 2020, each of which is incorporated herein by reference in its entirety.

Immunotherapies using immune checkpoint inhibitors (ICIs) have shown great promise in the treatment of various cancers, particularly including cancers characterized by solid tumors. Indeed, ICI therapy is standard of care for lung cancer, breast cancer, and certain other solid tumor types (See, Tang et al., “Comprehensive analysis of the clinical immuno-oncology landscape”, Ann. Oncol., 29, 2018; see also, Vaddepally et al., “Review of Indications of FDA-Approved Immune Checkpoint Inhibitors per NCNN Guidelines with the Level of Evidence”, Cancers (Basel), 12, 2020, each of which is incorporated herein by reference in its entirety). Although ICIs are able to improve clinical outcomes for patients with a variety of solid tumors, only a small subset of patients respond (See, Havel et al., “The evolving landscape of biomarkers for checkpoint inhibitor immunotherapy”, Nat Rev Cancer, 19, 2019; see also, Marshall et al., “Immuno-Oncology: Emerging Targets and Combination Therapies”, Front Oncol, 8, 2018, each of which is incorporated herein by reference in its entirety). Moreover, ICIs can cause immune-related adverse events, some of which are clinically serious and potentially life-threatening (See, Postow et al., “Immune-Related Adverse Events Associated with Immune Checkpoint Blockade”, N. Engl. J Med, 378, 2018; see also, Puzanov et al., “Managing toxicities associated with immune checkpoint inhibitors: consensus recommendations from the Society for Immunotherapy of Cancer (SITC) Toxicity Management Working Group”, J Immunother Cancer, 5, 2017, each of which is incorporated herein by reference in its entirety). The present disclosure addresses a need to identify patients who are more likely to benefit from ICI therapy with minimal toxicity.

There are currently a number of FDA-approved ICIs on the market that target PD-1, PDL-1, and CTLA-4 immune checkpoints (see Table 2 below). Immunomodulation therapy treatment with these ICIs has been approved and tested for a variety of indications, with scoring guidelines also available based upon the publicly available National Comprehensive Cancer Network (NCCN) scoring guidelines (see Tables 3-9 below). Dosage and usage information for each drug is also available within corresponding, publicly available FDA prescribing information.

TABLE 2
FDA-Approved ICIs

| | | Initial FDA | |
|---|---|---|---|
| Drug Name | Target | Approval Date | Dosage information |
| ipilimumab | CTLA-4 | 2011 | See page 1 of FDA |
| | | | prescribing information |
| nivolumab | PD-1 | 2014 | See page 1 of FDA |
| | | | prescribing information |
| pembrolizumab | PD-1 | 2014 | See pages 1-3 of FDA |
| | | | prescribing information |
| cemiplimab- | PD-1 | 2018 | See page 1 of FDA |
| rwlc | | | prescribing information |
| atezolizumab | PDL-1 | 2016 | See page 1 of FDA |
| | | | prescribing information |
| avelumab | PDL-1 | 2017 | See page 1 of FDA |
| | | | prescribing information |
| durvalumab | PDL-1 | 2017 | See page 1 of FDA |
| | | | prescribing information |

TABLE 3
Ipilimumab Indications and NCCN Guidelines. Adapted from Vaddepally et al.

| | NCCN Guideline |
|---|---|
| Indications | Category |
| Surgically unresectable, stage 3 or 4 malignant melanoma, | 2A |
| previously treated or untreated in adults and pediatric | |
| patients >12 years | |
| BRAF V600 wild-type unresectable or metastatic melanoma | 1 |
| In combination with nivolumab for unresectable or metastatic | 1 |
| melanoma across BRAF status | |
| Adjuvant treatment of cutaneous melanoma stage IIIA, IIIB, | 2A |
| and IIIC after complete resection along with total | |
| lymphadenectomy | |
| In combination with nivolumab, for patients with previously | 1 |
| untreated advanced renal cell carcinoma (RCC), relapse and | 2A |
| stage IV, with intermediate- or poor-risk RCC, regardless of | |
| PD-L1 | |
| This combination can be used in relapse and stage IV RCC | |
| patients as a subsequent therapy after patients have undergone | |
| TKI, VEGF or mTOR therapy | |
| In combination with nivolumab for microsatellite instability- | 2A |
| high (MSI-H) or mismatch repair deficient (dMMR) | |
| metastatic colorectal cancer that has progressed following | |
| treatment with fluoropyrimidine, oxaliplatin, and irinotecan in | |
| adults and pediatric patients >12 years | |

TABLE 4
Nivolumab Indications and NCCN Guidelines. Adapted from Vaddepally et al.

| | NCCN Guideline |
|---|---|
| Indications | Category |
| Unresectable or metastatic melanoma cancer progressed | 1 |
| following treatment with ipilimumab, or a BRAF inhibitor in | |
| BRAF mutation-positive patients | |
| In combination with ipilimumab for unresectable or | 1 |
| metastatic melanoma across BRAF status | |
| Lymph node-positive or metastatic melanoma patients who | 1 |
| had undergone complete resection | |
| Current first-line systemic therapy in patients with recurrent | 1 |
| or metastatic melanoma regardless of BRAF V600-mutation | |
| status | |
| Second line regardless of the histological subtype in non- | 1 |
| small-cell lung cancer (NSCLC) in patients who showed | |
| progression despite the platinum-based therapy | |
| Small-cell lung cancer (SCLC) patients who progressed on | 2A |
| platinum-based therapy and at least one other line of therapy | |
| Advanced renal cell cancer (RCC) with prior anti-cancer | 1 |
| therapy (mTOR) | |
| In combination with ipilimumab, for patients with previously | 1 |
| untreated advanced RCC, relapse and stage IV, with | 2A |
| intermediate- or poor-risk RCC, regardless of PD-L1 This | |
| combination can be used in relapse and stage IV RCC patients | |
| as a subsequent therapy after patients have undergone TKI, | |
| VEGF or mTOR therapy | |
| Hodgkin's lymphoma that has progressed or relapsed after | 2A |
| auto-HSCT and post-transplantation brentuximab vedotin | |
| therapy, or three or more lines of systemic therapy that | |
| includes auto-HSCT | |
| Recurrent or metastatic squamous cell cancer of head and | 1* |
| neck (SCCHN) that hasprogressed on or after platinum-based | 2B* |
| therapy (non-nasopharyngeal-Category 1*; | |
| nasopharyngeal-Category 2B*) | |
| Surgically unresectable or metastatic urothelial cancer | A |
| In combination with ipilimumab for microsatellite instability- | 2A |
| high (MSI-H) or mismatch repair deficient (dMMR) | |
| metastatic colorectal cancer that has progressed following | |
| treatment with fluoropyrimidine, oxaliplatin, and irinotecan in | |
| adults and pediatric patients >12 years | |
| Hepatocellular carcinoma (HCC) previously treated with | 2A |
| sorafenib | |

TABLE 5
Pembrolizumab. Indications and NCCN Guidelines.
Adapted from Vaddepally et al.

| | NCCN Guideline |
|---|---|
| Indications | Category |
| Metastatic melanoma refractory to ipilimumab and BRAF | 2A |
| inhibitor with BRAF mutation | |
| Previously untreated advanced melanoma regardless of BRAF | 2A |
| mutation status | |
| Adjuvant treatment of lymph node(s)-positive melanoma | 1 |
| following complete resection | |
| Metastatic melanoma with limited resectability, if there is no | 2A |
| disease after resection, as an adjuvant therapy | |
| Metastatic non-small-cell lung cancer (NSCLC) that | 1 |
| progressed after platinum-based therapy or, if appropriate, | |
| targeted therapy (EGFR/ALK mutation) and positive for | |
| PDL-1 | |
| First-line treatment in patients with metastatic non-small-cell | 1 |
| lung cancer with high PDL-1 expression (50%) but no EGFR | 2B if PDL-1 1-49% |
| or ALK mutation | |
| First-line treatment in combination with pemetrexed and | 1 |
| carboplatin for metastatic non-squamous NSCLC without | |
| EGFR or ALK mutation, irrespective of PDL-1 expression | |
| First-line treatment in metastatic squamous NSCLC in | 1 |
| combination with carboplatin with paclitaxel/nab-paclitaxel | |
| regardless of PD-L1 status | |
| First-line monotherapy in patients with stage 3 NSCLC who | 1 |
| are not candidates for surgical resection as well as | |
| chemoradiation or metastatic NSCLC with PDL-1 expression | |
| 1% and no EGFR or ALK mutation | |
| For recurrent or metastatic squamous cell cancer of head and | 1* |
| neck (HNSCC) patients with progression on standard | 2B* |
| platinum-based therapy (non-nasopharyngeal-Category 1*; | |
| nasopharyngeal and PD-L1 positive-Category 2B*) | |
| First-line therapy for patients with metastatic or unresectable, | 2A |
| recurrent HNSCC either as monotherapy in patients whose | |
| tumor expresses PD-L1 (combined positive score 1%) or in | |
| combination with platinum and fluorouracil | |
| Refractory adult and pediatric classical Hodgkin's lymphoma | 2A |
| Unresectable or metastatic urothelial cancer with progression | 2A |
| on or after platinum-based therapy including in the adjuvant | |
| setting | |
| First-line therapy for unresectable or metastatic urothelial | 2A |
| cancer patients who are ineligible for cisplatin-containing | |
| chemotherapy | |
| Locally advanced or metastatic urothelial carcinoma patients | 2A |
| who are not eligible for cisplatin-containing therapy and | |
| whose tumors express PD-L1 >10%, or in patients who are | |
| not eligible for any platinum-containing chemotherapy | |
| regardless of PD-L1 status | |
| Unresectable or metastatic solid tumor patients with | 2A |
| biomarker MSI-H or dMMR who have progressed after first- | |
| line therapy without satisfactory alternative therapy, | |
| irrespective of the location of the primary tumor | |
| Third-line therapy for recurrent locally advanced or metastatic | 2A |
| gastric or gastroesophageal junction (GEJ) adenocarcinoma | |
| patients with PD-L1 expression (combined positive score | |
| 1%) who have progressed on or after two or more prior lines | |
| of therapy including fluoropyrimidine and a platinum-based | |
| regimen and, if appropriate, HER2/neu-targeted therapy | |
| Esophageal (squamous and adenocarcinoma) and EGJ | 2A |
| adenocarcinoma, subsequent therapy for MSI-H or dMMR | |
| tumors; Category 2B for second-line therapy with PD-L1 | |
| expression 10% Category 2B for third-line or subsequent | |
| therapy | |
| Recurrent or metastatic cervical cancer progressing on or after | 2A |
| chemotherapy and positive for PDL-1 | |
| Refractory or relapsed primary mediastinal large B-cell | 2A |
| lymphoma (PMBCL) | |
| HCC patients who had previously been treated with sorafenib | 2B |
| First-line therapy for adult and pediatric patients with | 2A |
| recurrent or locally advanced or metastatic Merkel cell | |
| carcinoma (MCC) | |
| Combination with axitinib (Inlyta) as first-line treatment for | 1* |
| patients with metastatic renal cell cancer (RCC) (poor and | 2A* |
| intermediate risk-Category 1*; favorable risk-Category | |
| 2A*) | |

TABLE 6
Cemiplimab Indications and NCCN Guidelines. Adapted from Vaddepally et al.

| | NCCN Guideline |
|---|---|
| Indications | Category |
| Metastatic or locally advanced cutaneous squamous cell | 2A |
| carcinoma who are not the candidate for curative surgery or | |
| radiation | |

TABLE 7
Avelumab Indications and NCCN Guidelines. Adapted from Vaddepally et al.

| | NCCN Guideline |
|---|---|
| Indications | Category |
| Metastatic Merkel cell carcinoma of adults and pediatric | 2A |
| patients >12 years including those who have not received | |
| prior chemotherapy | |
| Locally advanced or metastatic urothelial carcinoma patients | 2A |
| whose disease progressed during or following platinum- | |
| containing chemotherapy or within 12 months of neoadjuvant | |
| or adjuvant platinum-containing chemotherapy | |
| Avelumab in combination with axitinib (Inlyta) for the first- | 2A |
| line treatment of patients with advanced renal cell carcinoma | |
| (RCC) alternative to pembrolizumab (which is the preferred | |
| agent) | |

TABLE 8
Durvalumab Indications and NCCN Guidelines. Adapted from Vaddepally et al.

| | NCCN Guideline |
|---|---|
| Indications | Category |
| Locally advanced or metastatic urothelial carcinoma patients | 2A |
| with disease progression during or following platinum- | |
| containing chemotherapy, or whose disease has progressed | |
| within 12 months of receiving platinum-containing | |
| chemotherapy neoadjuvant or adjuvant, alternative to | |
| preferred agent pembrolizumab | |
| Stage III non-small-cell lung cancer (NSCLC) patients for | 1 |
| surgically unresectable tumors and whose cancer has not | |
| progressed after treatment with chemoradiation | |

TABLE 9
Atezolizumab Indications and NCCN Guidelines. Adapted from Vaddepally et al.

| | NCCN Guideline |
|---|---|
| Indications | Category |
| Locally advanced or metastatic urothelial carcinoma with | 2A |
| disease progression during or following platinum-containing | |
| chemotherapy, or within 12 months of receiving platinum- | |
| containing chemotherapy as neoadjuvant or adjuvant therapy | |
| Locally advanced or metastatic urothelial carcinoma patients | 2A |
| who are not candidates for platinum-based chemotherapy | |
| regardless of PD-L1 expression | |
| Metastatic non-small-cell lung cancer (NSCLC) patients with | 1 |
| disease progression during or following platinum-containing | |
| chemotherapy who have progressed on an appropriate FDA- | |
| approved targeted therapy | |
| In combination with bevacizumab, paclitaxel and carboplatin | 1 |
| for initial treatment of people with metastatic non-squamous | |
| non-small-cell lung cancer (NSCLC) with no EGFR or ALK | |
| In combination with carboplatin and etoposide, for the initial | 1 |
| treatment of adults with extensive-stage small-cell lung | |
| cancer | |
| In combination with paclitaxel for adults with unresectable | 2A |
| locally advanced or metastatic triple-negative breast cancer in | |
| people whose tumors express PD-L1 | |

Combinations of ICI therapy with targeted therapeutics such as small molecule immunomodulators (e.g. colony stimulating factor-1 receptor (CSF-1R) and focal adhesion kinase (FAK)) and anti-angiogenesis (e.g. VEGF) inhibitors that act upon the tumor microenvironment are being investigated to improve durable response rates. See, for example, Osipov et al. “Small molecule immunomodulation: the tumor microenvironment and overcoming immune escape” J Immunother Cancer, 7:224, 2019; Ciciola et al. “Combining Immune Checkpoint Inhibitors with Anti-Angiogenic Agents” J Clin Med., 9(3): 675, 2020.

TABLE 10
Recent FDA Approvals for ICI therapy in combination with standard
of care chemotherapy or targeted therapeutics. Adapted from
cancerresearch.org/immunotherapy/timeline-of-progress.

| | FDA Approval |
|---|---|
| Description | Date |
| The FDA approved the combination of atezolizumab | May 29, 2020 |
| (Tecentriq), a PD-L1 checkpoint inhibitor, and bevacizumab | |
| (Avastin), a VEGF-A monoclonal antibody, for the treatment | |
| of patients with previously untreated hepatocellular | |
| carcinoma (HCC), the most common form of liver cancer. | |
| The FDA approved durvalumab (Imfinzi), a PD-L1 | Mar. 30, 2020 |
| checkpoint inhibitor immunotherapy, as a the first-line | |
| treatment of adult patients with extensive-stage small cell | |
| lung cancer (ES-SCLC) in combination with standard-of-care | |
| chemotherapy. | |

TABLE 11
Examples of Clinical Trials utilizing targeted therapeutics to act upon the TME
to improve immunomodulation. Adapted from Osipov et al. and Ciciola et al.

| Description | NCI Identifier |
|---|---|
| Evaluation of Safety and Activity of an Anti-PDL1 Antibody | NCT02777710 |
| (DURVALUMAB) Combined With CSF-1R TKI | |
| (PEXIDARTINIB) in Patients With Metastatic/Advanced | |
| Pancreatic or Colorectal Cancers | |
| A Study of ARRY-382 in Combination With Pembrolizumab | NCT02880371 |
| for the Treatment of Patients With Advanced Solid Tumors | |
| Phase I/II Study of BLZ945 Single Agent or BLZ945 in | NCT02829723 |
| Combination With PDR001 in Advanced Solid Tumors | |
| Study of FAK (Defactinib) and PD-1 (Pembrolizumab) | NCT02758587 |
| Inhibition in Advanced Solid Malignancies (FAK-PD1) | |
| ROCKIF Trial: Re-sensitization of Carboplatin-resistant | NCT03287271 |
| Ovarian Cancer With Kinase Inhibition of FAK | |
| Defactinib Combined With Pembrolizumab and Gemcitabine | NCT02546531 |
| in Patients With Advanced Cancer | |
| Study of Safety, Efficacy and Pharmacokinetics of CT-707 in | NCT02695550 |
| Patients With ALK-positive Non-small Cell Lung Cancer | |
| Study of Pembrolizumab With or Without Defactinib | NCT03727880 |
| Following Chemotherapy as a Neoadjuvant and Adjuvant | |
| Treatment for Resectable Pancreatic Ductal Adenocarcinoma | |
| Phase I/II Study of Nivolumab and Ipilimumab Combined | NCT03377023 |
| With Nintedanib in Non Small Cell Lung Cancer | |
| Combination Chemotherapy, Bevacizumab, and/or | NCT02997228 |
| Atezolizumab in Treating Patients With Deficient DNA | |
| Mismatch Repair Metastatic Colorectal Cancer, the COMMIT | |
| Study | |
| Study of First-line Pembrolizumab (MK-3475) With | NCT03898180 |
| Lenvatinib (MK-7902/E7080) in Urothelial Carcinoma | |
| Cisplatin-ineligible Participants Whose Tumors Express | |
| Programmed Cell Death-Ligand 1 and in Participants | |
| Ineligible for Platinum-containing Chemotherapy (MK-7902- | |
| 011/E7080-G000-317/ LEAP-011) | |

### T Cell Therapy

Among the immunomodulation therapies being developed and/or utilized to treat certain cancers are therapies that involve administration of populations of cells (typically T cells) that have been expanded ex vivo. Adoptive T cell therapies, including CAR-T therapies, have shown great promise in certain contexts. See, for example, Hinrichs & Restifo Nat Biotechnol 31:999, 2013; Newick et al Oncolytics 2016; Zhang & Wang doi.org/10.1177/1533033819831068, 2019. The present disclosure provides technologies that can improve effectiveness of T cell therapies, by providing tumor characterization technologies, and establishing parameters (e.g., correlations) indicative of tumor responsiveness to immunomodulation.

Chimeric antigen receptor (CAR)-T-cell therapy is a form of immunomodulation therapy that repurposes T cells to express specific protein components able to recognize surface-exposed antigens on cancer cells. Once bound to a target, the reprogrammed T cells activate and proceed to destroy the tumor cells through various mechanisms, including, e.g., stimulated cell expansion and enhanced cytokine production (See, Tang et al. “Therapeutic potential of CAR-T cell-derived exosomes: a cell-free modality for targeted cancer therapy”, Oncotarget, 6, 2015, incorporated herein by reference in its entirety). T cells may be harvested from a patient by leukapheresis and enriched through various positive and negative selection methods, including, e.g., elutriation, ex vivo expansion. Isolated T cell populations can be engineered ex vivo to express necessary CAR machinery, including, e.g., tumor-binding regions, which are often optimized to target cancer-specific surface antigens. These reprogrammed T cells can be further enriched to select for viable cells expressing the desired CAR activation and binding domains, e.g. through flow cytometry methods, including fluorescence-activated cell sorting (FACS).

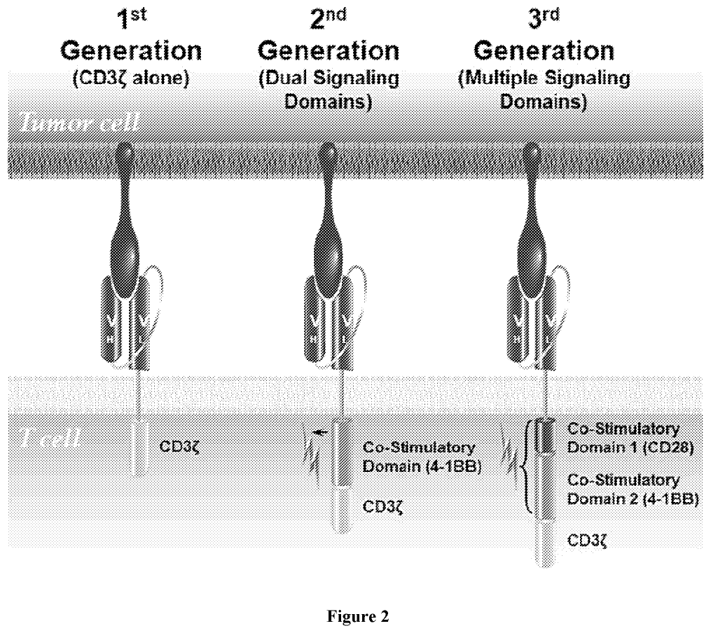

Engineered CAR-T cells typically comprise an extracellular domain for antigen recognition, which is connected to one or more intracellular signaling domains to control T-cell activation. An antigen recognition domain may consist of one or more antibody components, e.g. the variable heavy and variable light chains of an antibody, which are fused through a peptide spacer. A peptide spacer may be further linked to an intracellular signaling domain, such an immune-receptor-tyrosine-based-activation-motif (ITAM) protein. Recent work has shown that inclusion of one or more co-stimulatory domains can lead to improved T-cell activation, among other things (see FIG. 2). CAR-T cells may be harvested from a patient for self-use or collected from a healthy, allogeneic donor for use in a patient. See, Feins et al. “An introduction to chimeric antigen receptor (CAR) T-cell immunotherapy for human cancer”, Am J Hematol. 94, 2019, incorporated herein by reference in its entirety.

There are several FDA-approved CAR-T therapies currently available for treatment of certain B-cell lymphomas. These therapies include tisagenlecleucel (Kymriah™), axicabtagene ciloleucel (Yescarta™), and brexucabtagene autoleucel (Tecartus™). Dosage and usage information for each therapy is available within corresponding, publicly available FDA prescribing information.

### Neoantigen Vaccine Therapy

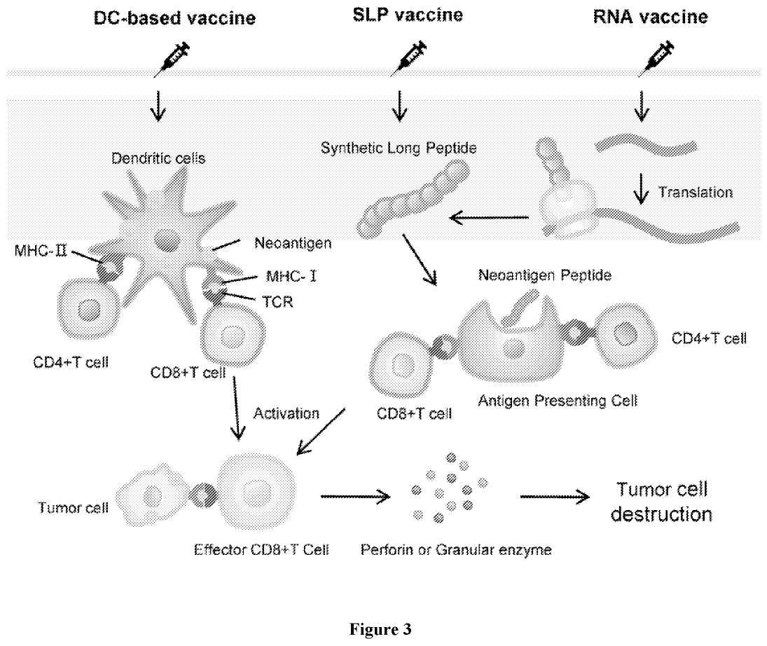

Neoantigens are cancer-specific epitopes that arise as a result of unique mutations within tumor cells. A variety of therapeutic modalities have been developed to trigger or enhance a patient's immune response to neoantigens that arise in his/her tumor. For example, a variety of prediction algorithms and/or characterization regimes have been developed to identify those neoantigens most likely to support a robust patient immune response, and vaccine technologies that administer peptides containing neoantigens, nucleic acids (e.g., DNA or RNA) that encode them, dendritic cells that display them, T-cells that target them, etc. have been the subject of many studies (See, for example, FIG. 3 below and Peng et al., “Neoantigen vaccine: an emerging tumor immunotherapy”, Mol. Cancer, 18, 2019; see also, Chu et al. Theranostics 8:4238, 2018, each of which is incorporated herein by reference in its entirety).

### Combination Therapy

In some embodiments, the present disclosure relates to administration (and/or monitoring, and/or withholding) of one or more combination therapies, typically including at least one immunomodulation therapy.

For example, according to the present disclosure, in some embodiments, administration of one therapy may increase responsiveness to another therapy (e.g., to an immunomodulation therapy).

Moreover, those skilled in the art are aware that combination therapy, including combinations of immunomodulatory therapies, is often recommended for cancer therapy.

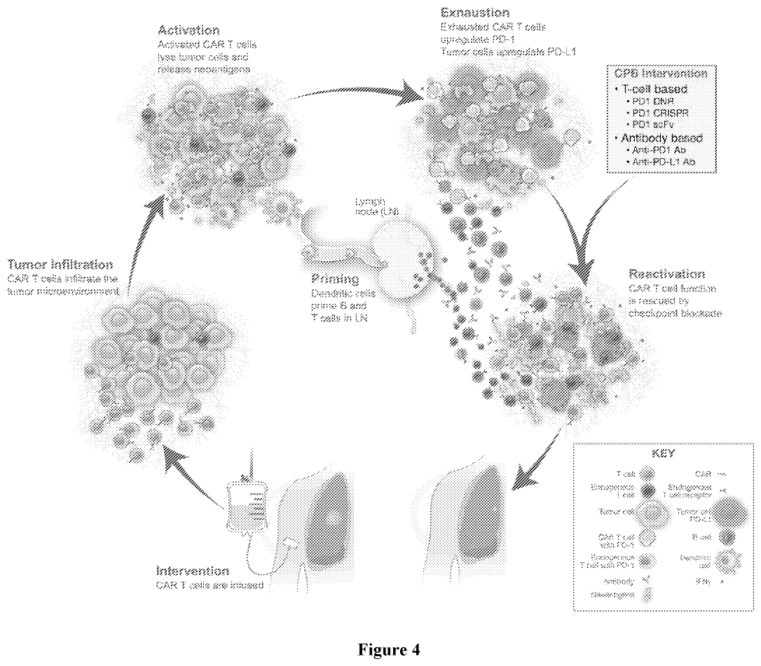

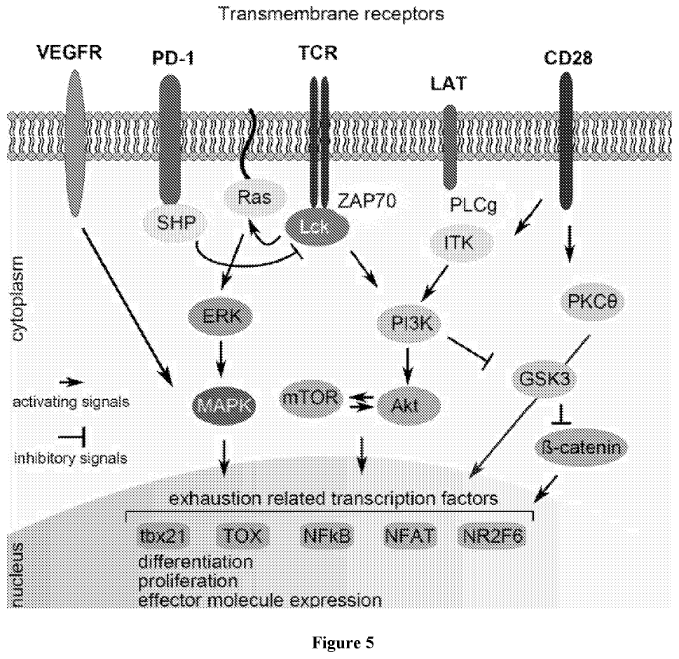

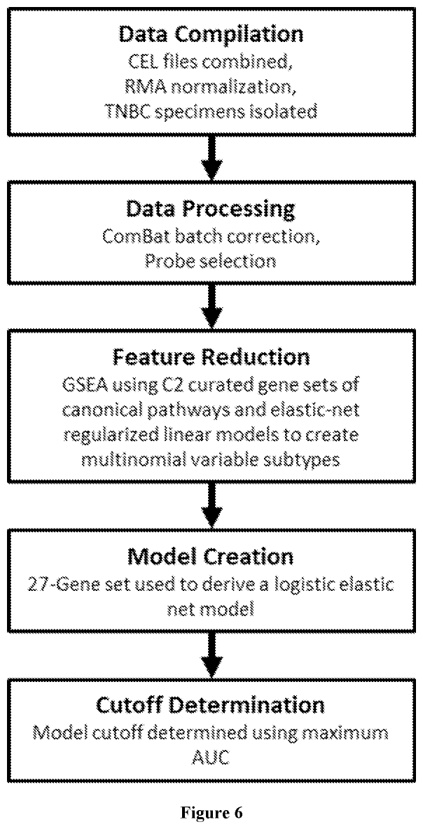

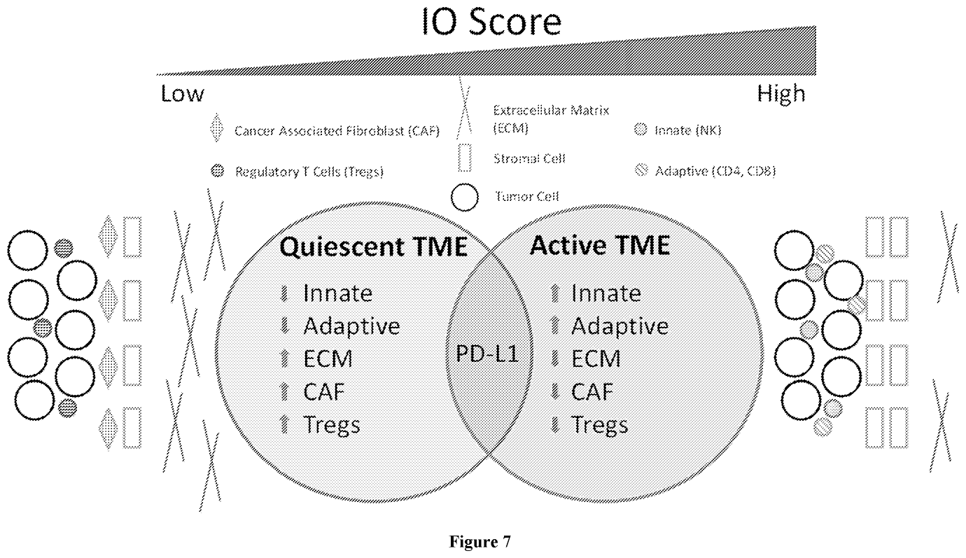

For example, combination of ICIs with CAR-T therapy has been proposed, among other things to address up-regulation of certain immune checkpoints that has been shown to correlate with tumor resistance to CAR-T cell therapy. (See, Beatty et al., “Chimeric antigen receptor T cells are vulnerable to immunosuppressive mechanisms present within the tumor microenvironment”, Oncoimmunology, 3, 2014, incorporated herein by reference in its entirety). Alternatively or additionally, combination of T cell and ICI therapy may address T-cell exhaustion reported with certain adoptive T cell (e.g., CAR-T therapies) after initial activation and lysis of tumor cells (See FIG. 4). Initial administration of CAR-T therapy followed by ICI treatment has been proposed as a strategy to induce reactivation of CAR-T function and produce functional therapeutic persistence (See, Grosser et al., “Combination Immunotherapy with CAR T Cells and Checkpoint Blockade for the Treatment of Solid Tumors”, Cancer Cell, 36, 2019, incorporated herein by reference in its entirety).

Additionally, pre-clinical studies have shown that combination therapies comprising an anti-CTLA-4 antibody and a tumor antigen-specific vaccine led to increased survival in a tumor cell model (See, Linch et al., “Combination OX40 agonist/CTLA-blockade with HER2 vaccination reverses T-cell anergy and promotes survival in tumor-bearing mice”, PNAS, 2016, incorporated herein by reference in its entirety). Various reports recommending combination of ICI therapy with neoantigen therapy have also been described. See, for example, Fotin-Mleczek et al. J Gene Med. 14(6): 428-39; see also WO2014/127917.

In some embodiments, provided technologies are applied to combination therapy with at least one immunomodulation therapy and at least one other therapy (e.g., chemotherapy, radiation therapy, surgical therapy, etc.).

For example, certain kinase inhibitors have been shown to enhance ICI therapy effects (See, Langdon et al., “Combination of dual mTORC1/2 inhibition and immune-checkpoint blockade potentiates anti-tumour immunity”, Oncoimmunology, 7, 2018, incorporated herein by reference in its entirety). Various pathways are known to interact with PD-1 signaling, for example, and could be targeted through co-administration of various therapeutics with ICIs (See FIG. 5).

Without wishing to be bound by a particular therapy, the present disclosure provides insights relating to tumor responsiveness that are applicable to various combination therapies. In some embodiments, a combination of one or more immunotherapies and/or anti-tumor therapies may be predicted to be effective when administered to particular patients identified as described herein and/or when administered in a particular order. In some embodiments, the present disclosure provides technologies for selecting patients to receive (or not) such combination therapy, and/or for monitoring such combination therapy (e.g., to assess likely continued effectiveness over time). In some embodiments, effectiveness is assessed or pre predicted relative to a particular comparator therapy (e.g., monotherapy).

### IO Scores for Immune Checkpoint Inhibitor Therapy

Given the importance of ICI therapy, significant effort has been invested in determining predictive biomarkers that can support patient selection for ICI therapy (i.e., that can discriminate between patients who are or are not likely to respond if treated with ICI therapy).

For example, several studies have investigated expression of programmed death-ligand 1 (PD-L1) on tumor cells as a potential predictive biomarker for responsiveness to therapy targeting PD-1 and/or PD-L1. Unfortunately, literature reports that PD-L1 testing does not consistently predict patient benefit from immunomodulation therapy (See, Gibney et al., “Predictive biomarkers for checkpoint inhibitor-based immunotherapy”, Lancet Oncol, 17, 2016; see also, Mehnert et al., “The Challenge for Development of Valuable Immuno-oncology Biomarkers”, Clin Cancer Res, 23, 2017; see also, Wojas-Krawczyk et al., “Beyond PD-L1 Markers for Lung Cancer Immunotherapy”, Int J Mol Sci, 20, 2019, each of which is incorporated herein by reference in its entirety).

The present disclosure identifies the source of a problem with many such efforts to identify sufficiently effective predictive biomarkers for ICI therapy to be useful in treating patient populations. For example, without wishing to be bound by any particular theory, the present disclosure proposes that complexity of the tumor-immune system interactions that characterize the tumor microenvironment (TME) can complicate efforts to develop such sufficiently effective biomarkers. Within the TME is a complex and dynamic milieu of non-malignant cells that interact with each other and with the tumor cells, affecting tumor growth, invasion and metastasis (See, Binnewies et al., “Understanding the tumor immune microenvironment (TIME) for effective therapy”, Nat Med, 24, 2018; see also, Butturini et al., “Tumor Dormancy and Interplay with Hypoxic Tumor Microenvironment”, 20, 2019, each of which is incorporated herein by reference in its entirety). The present disclosure proposes that a biomarker which is able to capture the complex interactions and signals of the TME could be more useful in selecting patients who are more likely to benefit from ICI therapies because multiple dimensions are assessed. Assessment of multiple biomarker dimensions can increase sensitivity and accommodate sampling error to produce more accurate results when working with limited sample sizes, e.g. limited amount of tumor tissue sample.

One approach to developing positive or negative immunomodulatory signatures that might be useful as biomarkers of responsiveness to ICI therapy involved clinical subtyping of triple negative breast cancer (TNBC) patients (See, Ring et al., “Generation of an algorithm based on minimal gene sets to clinically subtype triple negative breast cancer patients”, BMC Cancer, 16, 2016, incorporated herein by reference in its entirety). In particular, a 101-gene model was developed that classified TNBC into five molecular subtypes, including two basal like (BL1 and BL2), luminal androgen receptor (LAR), mesenchymal (M), and mesenchymal stem-like (MSL); with each of these subtypes further classified by a positive or negative immunomodulatory (IM) signature.

The present disclosure report provides an insight that TNBC tumors of the M subtype never had a positive IM signature, an observation that can now be appreciated to be consistent with studies showing that the M and IM subtypes are inversely correlated (See, Lehmann et al., “Refinement of Triple-Negative Breast Cancer Molecular Subtypes: Implications for Neoadjuvant Chemotherapy Selection”, PLoS One, 11, 2016; see also, Grigoriadis et al., “Mesenchymal Subtype Negatively Associates with the Presence of Immune Infiltrates within a Triple Negative Breast Cancer Classifier”, 2016, each of which is incorporated herein by reference in its entirety).

Without wishing to be bound by any particular theory, the present disclosure proposes that the M and MSL subtypes may be considered antithetical to the IM subtype, with the former subtypes indicating a more quiescent immunological state and the latter indicating an immunologically active state. Additionally, the present disclosure provides an insight that the molecular basis for the M, MSL, and IM subtypes can translate across other solid tumor types based on features of the TME driving this profile. The present disclosure describes technologies that it demonstrates are effective to develop a gene expression algorithm to measure a TME by optimizing a gene set to include those most relevant to the M, MSL, and IM subtypes. Among other things, the present disclosure provides an insight that strategies provided herein can distinguish tumors in an immunologically active (e.g., “hot”) state from tumors that are either: 1) in a more quiescent state and unlikely to respond (e.g., “cold”) to immunomodulation therapy (e.g. due to increased expression of signatures associated with M and MSL subtypes); and/or 2) in a more quiescent state yet poised to develop or enter an immunologically active state (e.g., to become immunologically “hot”), and therefore likely to respond to immunomodulation therapy (e.g. due to increased expression of signatures associated with IM subtype). These findings may well generalize across tumors (e.g, particularly across solid tumors) and therefore have expanded utility across multiple cancer types.

The present disclosure exemplifies effectiveness of provided technologies through development and validation of a new 27-gene immuno-oncology algorithm that measures the TME and generates an associated IO score predicting response to immunomodulation therapy treatment. This algorithm was optimized using genes expressed in both quiescent and immunologically active tumors and may be useful in predicting response to immunotherapies.

In some embodiments, genes assessed in a provided algorithm are associated with a positive IM signature and M and/or MSL subtypes. In particular embodiments, genes with a positive IM signature are characterized as being associated with increased innate immunity (e.g. increased tumor infiltrating lymphocyte and/or natural killer cell levels) and/or adaptive immunity (e.g. increased CD4, CD8 levels) as well as decreased inflammatory characteristics (e.g. decreased neutrophil and/or regulatory T-cell levels). In some embodiments, genes with an M subtype are characterized as having increased expression of one or more of: (1) markers of epithelial-to-mesenchymal transition (EMT); (2) factors (e.g., secreted factors) that may recruit cancer-associated fibroblasts (CAFs) to the stroma. In some embodiments, genes with an MSL subtype are characterized as expressing 1) markers of cancer-associated fibroblasts (CAFs); and 2) markers of mesenchymal stem cells (MSCs), relative to a reference. In some embodiments, inclusion of independent IM, EMT, CAF, and MSC signatures ensures accurate algorithm scoring when making prognostic or predictive responses to immunomodulation therapy.

Among other things, the present disclosure documents a variety of advantages provided by technologies described herein, including the exemplified small gene set (i.e., 27-gene) immuno-oncology algorithm.

For example, the ability to define small (e.g., about 10 to about 50, or even about 10 to about 30) gene sets effective to achieve subtype classification and/or responsiveness prediction as described herein dramatically improves commercial feasibility. Moreover, application across cancers provides unusual and unexpected versatility.

The present disclosure addresses a previously unmet need for improved biomarkers to optimize ICI immunomodulation therapy use in clinical settings. Provided small gene set algorithms (e.g., the exemplified 27-gene immuno-oncology algorithm) can distinguish patients likely to benefit from treatments such as ICIs. Unlike previously described biomarker models, provided technologies measure the immunological state of the TME as a means to capture the interplay of the patient's immune system and tumor immune evasion. The concept that “tumors are wounds that do not heal” has been used to describe this interplay as the tumor co-ops the wound healing response which encompasses immunosurveillance as well as various aspects of wound healing that appear to be components of tumor maintenance and growth (See, Dvorak et al., “Tumors: wounds that do not heal-redux”, Cancer Immunol Res, 3, 2015, incorporated herein by reference in its entirety). Without wishing to be bound by any particular theory, we propose that provided strategies uniquely capture aspects of immunosurveillance, immunosuppression, and immune evasion as a tumor transitions from a proliferative to a metastatic state, thereby enabling for effective and accurate prediction.

In some embodiments, provided gene sets and/or algorithms may include and/or focus on genes associated with IM, EMT, CAF, and MSC signatures, optionally in preference to or even with exclusion of other markers (e.g. various growth factors), which can regulate many different cellular functions and provide confounding effects on scoring.

Another advantage of provided technologies include their ability to utilize data obtained from any of a variety of platforms.

In some embodiments, technologies described herein have improved predictive power through measurement of each of IM, M, and MSL signatures rather than a single marker group.

In some embodiments, technologies herein measure each of IM, M, and MSL signatures relative to a reference threshold (e.g., relative to the expression of an alternate set of genes, etc.). In some embodiments, a reference threshold may be determined through analysis of patient data (e.g., relative to patterns of gene expression compared to a pre-determined clinical standard).

Without wishing to be bound by any particular theory, we propose that, by measuring the immunological state of the TME as a whole, technologies described herein (e.g., including the exemplified 27-gene algorithm) may offer independent and incremental predictive value over the current gold standard biomarkers in the clinic.

### Other Features or Characteristics

In some embodiments, patients assessed or selected (e.g., to receive [or not] particular therapy) in accordance with the present disclosure may be characterized by one or more features and/or characteristics other than (e.g., in addition to) a particular IO score.

In some embodiments, features and characteristics assessed in accordance with the present disclosure may include one or more of cancer type (e.g. tissue type and/or histology of a tumor), prior lines of treatment received, age, and/or circulating tumor cell burden.

### Monitoring Over Time

In some embodiments, assessment of one or more particular features and/or characteristics (e.g., IO score and/or other characteristics or features) is performed with respect to the same patient at a plurality of different time points. In some embodiments, assessment of one or more particular features and/or characteristics is performed with respect to a particular patient prior to initiation of a particular therapeutic regimen and/or prior to administration of a particular dose of therapy in accordance with such therapeutic regimen.

For example, in some embodiments, features and/or characteristic assessment(s) is/are performed with respect to a subject or subjects who is receiving, has received, or is a candidate to receive immunomodulation therapy (e.g., with an ICI). In some embodiments, one or more features and/or characteristics is assessed prior to administration of such immunomodulation therapy. In some embodiments, one or more features and/or characteristics is assessed after administration of one or more doses of such immunomodulation therapy. In some embodiments, one or more features and/or characteristics is assessed prior to administration of immunomodulation therapy, and one or more features and/or characteristics is assessed after administration of one or more doses of immunomodulation therapy.

In some embodiments, different features and/or characteristics may be assessed at different times. In some embodiments, a plurality of features and/or characteristics may be assessed at the same time, and optionally others may be assessed at a different time.

In some embodiments, one or more features and/or characteristics may be assessed at multiple times. In some embodiments, at least one feature and/or characteristic may be assessed only a single time and one or more other feature(s) and/or characteristic(s) may be assessed at multiple times.

In some embodiments, provided technologies identify and/or select a subject or subject(s) to whom immunomodulation therapy (e.g. ICI therapy) is administered. Alternatively or additionally, in some embodiments, provided technologies determine timing for administration of one or more doses (which may, in some embodiments, be the same dose or may be different doses) of such immunomodulation therapy. In some particular embodiments, provided technologies determine timing for administration of one or more doses of such immunomodulation therapy relative to one or more doses of another therapy (e.g. chemotherapy).

In some embodiments, such monitoring of features and/or characteristics over time may inform decisions to continue or modify particular therapy, to interrupt or terminate such therapy, and/or to initiate alternative therapy.

In some embodiments, without wishing to be bound by any particular theory, assessment of one or more particular features and/or characteristics (e.g., IO score and/or other characteristics or features) affirms a quiescent TME (cold), might indicate that agents which modify or stimulate the immune response through stromal derived signals might be beneficial. Such agents may include, but are not limited to, focal adhesion kinase (FAK) inhibitors, anti TGF-beta, anti angiogenesis (e.g. VEGF, or other multi-targeted receptor tyrosine kinase (RTK) inhibitors and other vascular normalization agents), therapies which target the CD73-adenosine axis (e.g. CD73 inhibitors), other small molecule immunomodulation therapies (e.g. CSF1 Receptor inhibitors), traditional chemotherapies and MTOR inhibitors, bispecific molecules and antibodies, metabolic sequestration agents, and anti TIGIT therapies.

In some embodiments a low IO score implies that a patient is less likely to respond to ICI therapy and/or that a patient should consider alternate therapies guided by standardized consensus guidelines such as the NCCN guidelines, and or consider treatments offered in the context of an ongoing clinical trial.

### Algorithm Development

Elastic-net regularized linear models were employed to create individual subclassifying models for the BL1, BL2, LAR, MSL, M, and IM subtypes with the subtypes treated as a multinomial variable. The genes utilized for the M and IM subtype classifications with this model were then used to derive a logistic elastic net model on the new data set, minus three genes whose probes had been reassigned between analyses. Strength of association with classification variables was assessed using ten-fold cross validation of the misclassification error. The model threshold for determining the immuno-oncology score (IO score) was determined using the maximum area under the curve (AUC), in contrast to the significance of the correlation method for determining threshold previously described by Ring et al.

Without wishing to be bound by any particular theory, we note that one differentiated feature of the way this signature was developed was that it was a robust classifier first, and the association of the three features (M, IM, MSL) and their association with ICI (and other immune therapies) discovered later. The robust ability to classify, independent of knowing the biologic significance of classes, allows seamless translation between tumors of different tissue of origin. For example, a classifier can be trained on any gene expression dataset for a cancer of interest (e.g., a solid tumor cancer such as, for example, bladder, breast, cervical, colon, endometrial, kidney, lip, liver, lung (small cell or non-small cell), melanoma, mesothelioma, oral, ovarian, pancreatic, prostate, rectal, sarcoma, thyroid, etc.) and then, after its ability to define, detect, and/or distinguish subtypes of the relevant cancer is established, assess its correlation with responsiveness to particular therapy (e.g., ICI therapy).

In some embodiments, one or more genes (e.g., genes not included in a classifier or otherwise of interest) can be assessed through an established classifier in order to determine association with one of the three features (M, IM, MSL). For example, in some embodiments, these additional genes of interest can be added to an existing classifier gene set (e.g., the 27 gene set described herein, the 939 gene set described in Example 9) and association with the three features (M, IM, MSL) can be assessed through cluster analysis.

As described herein, among other things, the present disclosure provides effective classification of M, IM, and MSL features. Those skilled in the art, reading the present disclosure will therefore appreciate that it permits assessment of association (e.g., correlation) with these classified features. Thus, the present disclosure permits identification and/or characterization of other parameters (e.g., gene expression, gene mutation, protein expression, protein modification, epigenetic modification, etc.) that so associate. In some embodiments, such associated features may be or comprise biomarkers (e.g., that may act as a proxy for M, IM and/or MSL features, and therefore, in some embodiments, for likelihood of responsiveness to immunomodulation therapy) that may be detected, for example to characterize subject(s) prior to administration of immunomodulation therapy (e.g., to assess likelihood of responsiveness and/or to select for receipt of immunomodulation therapy and/or for alternative therapy) and/or to monitor subject(s) receiving immunomodulation therapy (e.g., for continued responsiveness and/or for development of resistance). Moreover, those skilled in the art, reading the present disclosure will appreciate that, in some embodiments, technologies provided by the present disclosure, by permitting assessment of association with M, IM, and/or MSL features, can reveal presence and/or development of biological event(s) (e.g., expression and/or mutation of a particular gene or genes) that recommend particular therapy (e.g., targeting a particular expressed or mutated gene) be utilized in addition or as an alternative to immunomodulation therapy.

The present disclosure demonstrates that use of unsupervised cluster analysis can facilitate identification of distinct biologic phenotypes that may each contribute to classification in any individual tumor specimen. Without wishing to be bound by any particular theory, we propose that this strategy may enhance biologic prediction of response to therapy (e.g., to IO therapy) in some samples; alternatively or additionally, this approach may increase sensitivity, for example by allowing some redundancy in detecting the immune status. For example, as noted above, non-surgical biopsies can be very sparse and stochastic sampling error risks missing relevant biology (e.g. TILS). The redundancy of measuring phenotype from multiple compartments may accommodate sampling error and give accurate results on more sparse specimens.

For at least these reasons, those skilled in the art will appreciate that features of algorithm development described herein are likely applicable across cancer types (e.g., for solid tumor cancers).

### Use

Technologies provided herein are useful in the assessment of tumor samples and/or for the development and/or validation of tumor subtype classifiers and/or predictors of responsiveness to therapy.

### Assessment of Tumor Samples

For example, with respect to assessment of tumor samples, a tumor sample of interest (e.g., a sample of a solid tumor such as for example, a skin, breast, lung, head and neck, gastric, renal, bladder, urothelial, bone, prostate, thyroid, or pancreatic tumor) may obtained and/or gene expression data from such a sample is obtained for analysis.

Those skilled in the art are aware of appropriate technologies for obtaining and preparing tumor samples, and for obtaining gene expression data from such samples. For example, gene expression assessment technologies include, but are not limited to microarray analysis, reverse transcription polymerase chain reaction (RT-PCR), Northern blot, reporter genes, real-time PCR, fluorescent in situ hybridization, hybridization detection, RNA-sequencing, and serial analysis of gene expression (SAGE).

In some embodiments, a tumor sample is from a patient prior to initiation of therapy (i.e., the sample is from a patient who has not received therapy to treat the tumor). In some embodiments, a tumor sample is from an excised tumor (e.g., a tumor that has been removed by surgery). In some embodiments, a tumor sample is a tumor biopsy. In some embodiments, the tumor sample is a liquid (e.g., is or comprises one or more of CNS fluid, blood, plasma, pleural fluid, serum, sweat, tears, urine, etc.; most typically blood, plasma, and/or serum.

In some embodiments, a tumor sample is from a patient who is receiving therapy (e.g., anti-cancer therapy which, in some embodiments, does not include and/or has not included ICI therapy and in other embodiments is or comprises ICI therapy).

In some embodiments, as discussed above, multiple tumor samples may be obtained from a patient (and/or from a particular tumor in a patient) over time, for example, to assess effectiveness of therapy and/or to assess continued likely responsiveness to therapy.

In some embodiments, one or more therapies (e.g., ICI therapy) are administered (or continued) for patients determined to have an IO score indicative of likely responsiveness as described herein. In some embodiments, one ore more therapies (e.g., ICI therapy) are withheld, or additional or alternative therapies are administered for patients determined to have an IO score indicative of likely non-responsiveness, or of a decrease in likely responsiveness over time. In some embodiments, additional or alternative therapies may comprise therapies associated with one or more genes, gene mutations and/or gene pathways identified (e.g., as described herein or otherwise) to be associated with a reduced IO score (e.g., associated with M or MSL classifiers). In some embodiments, IO score is re-assessed after administration of additional or alternative therapies. In some embodiments, IO score is monitored over time, for example to determine whether likely responsiveness to one or more therapies (e.g., ICI therapy) may change.

Algorithm Development and/or Assessment

As discussed herein, the present specification provides technologies for algorithm development and/or assessment. Included within such provided technologies are systems for validating and/or otherwise characterizing tumor subtype classifiers and/or predictors of responsiveness to therapy, for example by comparison with those described herein.

As described herein, the present disclosure documents effective classification of tumor (e.g., solid tumor, e.g., TNBC tumor) subtypes; provided classification technologies (e.g., the small gene set model described herein) provide a reference relative to which alternative embodiments or strategies can be compared; in some embodiments, the present disclosure thus provides methods that involve such comparison.

In some embodiments, technologies provided herein are useful for the determination of patterns of gene expression (e.g., identification of genes whose quantitative variation in expression may vary in similar ways across large sample sets, also referred to herein as metagenes). In some embodiments, metagenes may be used as classifiers to measure sample physiology by identifying physiologically significant subsets of samples (e.g., acting as diagnostics to support clinical decision making, including treatment selection). In some embodiments, one or more genes within a metagene group may be used to measure physiology. In some embodiments, two or more genes within a metagene group may be used to measure physiology. In some embodiments, three or more genes within a metagene group may be used to measure physiology. In some embodiments, a selected number of genes within a metagene group that is representative of the group as a whole may be used to measure physiology.

Analogously, the present disclosure documents effective prediction of likely tumor responsiveness to therapy; these technologies also provide a reference relative to which alternative embodiments or strategies can be compared; in some embodiments, the present disclosure thus provides methods that involve such comparison.

### EXEMPLIFICATION

### Example 1: Materials and Methods

### Data Analysis

All analyses, unless otherwise stated, were done on RStudio Version 1.2 utilizing R version 3.6 (See, RStudio Team, “Rstudio: Integrated Development for R”, 2019; see also, R Core Team, R: “A language and environment for statistical computing”, 2020).

### Algorithm Development

Elastic net regularized linear net models can be employed to create individual subclassifying models for BL1, BL2, LAR, MSL, M, and IM subtypes with each independent subtype treated as a multinomial variable. Genes utilized for the M and IM subtype classifications within this model can then be used to derive a logistic elastic net model on the new data set, removing genes whose probes are reassigned between analyses. Strength of association with classification variables can then be assessed using ten-fold cross validation of misclassification error. Model threshold for determining immuno-oncology (IO) score can be determined using maximum area under the curve (AUC). In some embodiments, model threshold for determining immuno-oncology (IO) score may be adjusted for particular patients and/or tumor samples. For example, in some embodiments, model threshold may be increased for patients with tumors that are particularly aggressive (e.g., requiring high level of treatment efficacy within a shortened time period). In some embodiments, model threshold may be decreased if one or more therapies of interest (e.g., ICI therapy) has low toxicity when administered to a subject and/or subject tissue. In some embodiments, model threshold may be adjusted to account for one or more therapies already administered and/or currently being administered to a subject. In some embodiments, model threshold may be adjusted to account for one or more additional therapies (e.g., non-ICI therapies) already administered and/or currently being administered to a subject. For example, in some embodiments, model threshold may be adjusted to account for one or more additional non-ICI therapies already administered and/or currently being administered prior to an ICI therapy.

### Gene Expression Dataset Processing

Twenty-five gene expression profile data sets, representing three microarray platforms, were downloaded from the publicly available Gene Expression Omnibus (GEO, ncbi.nlm.nih.gov/geo/). Data were combined from raw microarray expression (CEL) files collectively normalized by robust multiarray average (RMA), and log transformed. Samples from this data set were pared down to triple negative status using a bimodal distribution of ESR1, ERBB2, and PGR genes, resulting in 1284 unique TNBC samples. Of these, 994 unique TNBC samples were used to train the model, and the remaining 335 unique TNBC samples were used for model validation.

For genes represented by multiple probes, the probe with the highest interquartile range was selected to prioritize genes with a large dynamic range of expression. Batch correction was performed using an Empirical Bayes method, ComBat (See, Johnson et al., “Adjusting batch effects in microarray expression data using empirical Bayes methods”, Biostatistics, 8, 2007, incorporated herein by reference in its entirety). Patient datasets were previously made publicly available under the ethical policies of the National Institutes of Health's Gene Expression Omnibus (GMO) database. No additional ethics review was required for the in-silico analysis of these datasets.

TABLE 12
Source of TNBC specimens for Training and Validation

| Dataset | TNBC Specimens |
|---|---|
| GSE1456 | 44 |
| GSE1561 | 21 |
| GSE2034 | 59 |
| GSE2109 | 55 |
| GSE2603 | 35 |
| GSE2990 | 11 |
| GSE3494 | 27 |
| GSE3744 | 17 |
| GSE5327 | 35 |
| GSE5364 | 36 |
| GSE5462 | 2 |
| GSE6596 | 8 |
| GSE7390 | 42 |
| GSE7904 | 17 |
| GSE10780 | 5 |
| GSE11121 | 21 |
| GSE12093 | 57 |
| GSE12763 | 5 |
| GSE13787 | 10 |
| GSE16716 | 62 |
| GSE25066 | 178 |
| GSE31519 | 67 |
| GSE58812 | 107 |
| GSE76124 | 198 |
| GSE76250 | 165 |

### Model Building

Model building for the 27-gene immuno-oncology algorithm was performed using R version 3.5.2 (FIG. 6). The 101-gene signature was used to identify gene sets that distinguished the classes via gene set enrichment analysis (GSEA) using the C2 curated gene sets of canonical pathways (See, Subramanian et al., “Gene set enrichment analysis: a knowledge-based approach for interpreting genome-wide expression profiles”, PNAS, 102, 2005, incorporated herein by reference in its entirety). Elastic-net regularized linear models were employed to create individual subclassifying models for the BL1, BL2, LAR, MSL, M, and IM subtypes with the subtypes treated as a multinomial variable (See, Friedman et al., “Regularization Paths for Generalized Linear Models via Coordinate Descent”, J Stat Softw, 33, 2010, incorporated herein by reference in its entirety). The 30 genes utilized for the M and IM subtype classifications with this model were then used to derive a logistic elastic net model on the new data set, minus three genes whose probes had been reassigned between analyses. Strength of association with classification variables was assessed using ten-fold cross validation of the misclassification error. The model threshold for determining the immuno-oncology score (IO score) was determined using the maximum area under the curve (AUC) (See, Hajian-Tilaki et al., “Receiver Operating Characteristic (ROC) Curve Analysis for Medical Diagnostic Test Evaluation”, 4, 2013, each of which is incorporated herein by reference in its entirety), in contrast to the significance of the correlation method for determining threshold previously described by Ring et al . . .

### GSE81838 Dataset Analysis of TNBC Tumor Epithelial and Adjacent Stromal Tissue

Microarray data was obtained from GSE81838 where laser-capture microdissection had been performed on 10 TNBC tumors to isolate malignant epithelial cell-enriched areas and the adjacent stromal cell-containing areas of the tumor sections (See, Lehmann et al. “Refinement of Triple-Negative Breast Cancer Molecular Subtypes: Implications for Neoadjuvant Chemotherapy Selection”, 11, June 2016, incorporated herein by reference). The IO scores for each sample were obtained and correlated between the matched tumor epithelial and adjacent stromal tissue using Spearman's method.

### TCGA Breast Cancer Datasets and Analysis

Gene expression profiles from breast cancer specimens collected for The Cancer Genome Atlas (TCGA) were obtained from the National Cancer Institute Genomic Data Commons Data Portal. TNBC status was confirmed by bimodal modeling of ESR1, PGR, and ERBB2 gene expression, resulting in 180 total samples with matching tumor infiltrating lymphocytes (TILs) presence and intensity as described in Lehmann et al. Neutrophil presence was obtained by the TCGA study investigators and aligned to the TNBC samples. The IO scores of samples with intense TIL staining and samples with neutrophil presence of 30% or greater was assessed by the Welch t-test for significance.

### GEO Non-Small Cell Lung Cancer (NSCLC) Datasets and Analysis

The clinical response to immunomodulation therapy and expression data of NSCLC patients in the GSE135222 (27 patients) and GSE126044 (16 patients) cohorts was obtained from GEO. Response was measured in both cohorts using Response Evaluation Criteria in Solid Tumors (RESCIST) metrics, where patients exhibiting partial response or stable disease for >6 months were classified as responders (See, Schwartz et al., “RECIST 1.1-Update and clarification: From the RECIST committee”, Eur J Cancer, 62, 2016; see also, Jung et al., “DNA methylation loss promotes immune evasion of tumours with high mutation and copy number load”, Nat Commun, 10, 2019; see also, Kim et al., “Single-cell transcriptome analysis reveals TOX as a promoting factor for T cell exhaustion and a predictor for anti-PD-1 responses in human cancer”, Genome Med, 12, 2020; each of which is incorporated herein by reference in its entirety). Because response was defined in the same manner for both cohorts, we were able to combine the data for purposes of the analysis. Expression data from the combined cohort were processed using the 27-gene algorithm and analyzed by IO score. The difference in IO score between responders and non-responders was evaluated for significance using the Welch t-test. The data from the combined cohort was then evaluated for the correlation of IO score to objective response. The predefined threshold was used to divide patients into IO score positive and negative and compared to objective response to calculate an odds ratio.

### Example 2: Distinguishing Quiescent from Active Tumor Microenvironment

The present Example describes technologies for distinguishing quiescent from active tumor microenvironments through assessment of certain gene expression patterns or characteristics. In particular, the present Example describes determination of an IO score for a particular tumor sample, as reflective of the quiescent or immunologically active state of the TME. As described herein, without wishing to be bound by any particular theory, we propose that a negative IO score may indicate a quiescent state, where the tumor cells are more actively promoting angiogenesis, inducing an inflammatory response, and stimulating cancer-associated fibroblasts which collectively is constructing extracellular matrix. By comparison, a positive IO score may indicate one or more of: 1) a tumor poised to transition to an immunologically active TME (e.g. upon administration of an ICI); and 2) an immunologically active TME with reduced inflammatory characteristics combined with an increase in the innate and adaptive immune systems increasing tumor cell invasion. Further, using the IO score as a continuous variable may be predictive to the intensity and durability of response and correlate with clinical efficacy (e.g, objective response). Whereas a biomarker, e.g. an immune checkpoint receptor such as PD-L1, may be present in both states, the present disclosure describes development of small gene set(s)—such as the 27-gene algorithm described herein-able to distinguish a quiescent from an active TME.

### Example 3: Concordance Between IO Score and IM Status

The present Example confirms that IO scores determined using the 27-gene immuno-oncology algorithm correlate with IM scoring statuses from a previous 101-gene model. An independent expression-based centroid model, defined by M and IM features of a previous 101-gene model, were obtained through elastic net modeling to produce a total of 27 genes. These 27 genes were combined in an independent algorithm to generate IO scores corresponding to likelihood of response to immunomodulation therapy. The 27-gene immuno-oncology algorithm was compared to the previous 101-gene model through validation of 335 unique TNBC samples, resulting in 88% concordance for IO+/IM+ and IO−/IM− scores, as shown in Table 13 below.

TABLE 13
Concordance between IM status from the 101-gene model and
IO score from the 27-gene immuno-oncology algorithm within
the validation cohort of 335 unique TNBC samples.

| | | 101-gene | | | |
|---|---|---|---|---|---|
| | | IM+ | | IM− | |
| 27- | IO+ | 82 | (24%) | 37 | (11%) |
| gene | IO− | 2 | (1%) | 214 | (64%) |

### Example 4: Correlation of IO Score to Tumor Epithelial and Adjacent Stromal Tissue in TNBC

The present Example demonstrates that IO scores determined in accordance with the present disclosure can serve as a measure of the tumor microenvironment (TME) spanning tumor and stromal regions.

IO Scores were calculated for matched TNBC tumor epithelial and adjacent stromal tissue samples in the GSE81838 dataset. Due to low sample size (20 samples from 10 patients), IO scores for matched tumor epithelial and adjacent stromal tissue samples were calculated using Spearman's method. Correlation of IO scores between tissue types was calculated to be 92.7% (p<0.001) when matched to each patient, suggesting that IO score is a measure of TME spanning at least tumor and stromal regions.

### Example 5: IO Scoring of TNBC Samples with TILs or Neutrophils

The present Example demonstrates that IO scores determined in accordance with the present disclosure can correlate with levels of tumor infiltrating lymphocytes (TILs) and neutrophils. High levels of TILs may indicate an active immunological state and improved outcome after immunomodulation therapy, while increased levels of neutrophils may correspond to a quiescent immunological state and reduced response to immunomodulation therapy. IO Scores were evaluated for samples obtained from The Cancer Genome Atlas (TCGA), including triple negative breast cancer (TNBC) samples with high TILs and samples with increased neutrophil load. A statistically significant (FIG. 2, p=0.0092) difference in IO score was seen between TNBC samples with high TILs (IO Score=0.09) and samples with increased neutrophil load (IO Score=−0.30), indicating that a positive IO Score may possess features associated with a positive outcome after immunomodulation therapy while a negative IO Score may indicate poor immunomodulation therapy response.

### Example 6: Correlation of IO Score to Immunomodulation Therapy Response in NSCLC Patients

The present Example demonstrates that IO scores determined in accordance with the present disclosure can indicate potential response to immunomodulation therapy. IO Scores were evaluated for a combined cohort of non-small cell lung cancer (NSCLC) patients, where response to immunomodulation therapy was defined as exhibiting partial response or stable disease for at least 6 months. Average IO score for responders (IO Score=0.29) and non-responders (IO Score=−0.096) was found to be significantly by the Welch t-test (FIG. 3, p=0.0035).

### Example 7: Correlation of Mesenchymal Score to Focal Adhesion Kinase (FAK) Inhibitor Sensitivity in NSCLC Xenografts

The present Example demonstrates that using the 27-gene immuno-oncology algorithm described herein it is possible to predict sensitivity to FAK inhibitor drugs which may subsequently be used for immunomodulation of the TME. Adenocarcinoma xenograft model data were attained from GSE109302 and assessed by the 27-gene immuno-oncology algorithm. Of the 10 NSCLC cell lines, five were resistant and five were sensitive to the drug BI 853520. The average mesenchymal score for the resistant group was 0.076 and the sensitive group was 0.358 (p=0.025). Without wishing to be bound by any particular theory, these data demonstrate it may be possible to identify patients who will benefit from drugs which act upon the TME to improve immunomodulation (e.g., by pushing a “poised” tumor into a “hot” state as described herein), either alone or in combination with ICIs.

### Example 8A: Exemplary Gene Sets

In some embodiments, a gene set for use in accordance with the present disclosure comprises at least one gene from the following group:
Group A: CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNF AIP8, TNFSF10, COL2A1, FOXC1, KRT16, MIA, SFRP1, APOD, ASPN, HTRA1.

In some embodiments, such a gene set may include all genes from Group A.

### Example 8B: Exemplary Gene Sets

In some embodiments, a gene set for use in accordance with the present disclosure includes at least one gene from each of the following groups:
Group B1: CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10;
Group B2: COL2A1, FOXC1, KRT16, MIA, SFRP1;
Group B3: APOD, ASPN, HTRA1;

In some embodiments, such a gene set may include at least one gene from each of Group B1 and Group B2, and more than one gene from Group B3. In some embodiments, such a gene set may include at least one gene from each of Group B2 and Group B3, and more than one gene from Group B1. In some embodiments, such a gene set may include at least one gene from each of Group B1 and Group B3, and more than one gene from Group B2.

### Example 8C: Exemplary Gene Sets

In some embodiments, a gene set for use in accordance with the present disclosure includes at least one gene from each of the following groups:
Group C1: SAMSN1, CD80, CLEC7A, PDCD1LG2, CD274, S100A8, KYNU, LINC02195, IL9R, DUSP5;
Group C2: TNFAIP8, TNFSF10;
Group C3: RARRES3, APOL3, LINC02446, ZNF683, IFNG, FASLG;
Group C4: CD48, CD52, C16orf54, TESPA1, JAML, GMFG, ARHGAP15, TMEM273;
Group C5: CD3G, TIGIT, SIRPG, TRAC, CD3E, CD2, TRBV28, CD3D, TRBC2, CCR5, CD8A, CCL5, IL2RB, CXCR6;
Group C6: KMO, SNX10, PIK3AP1, SLC7A7, VCAM1, RASSF4, TFEC, HAVCR2;
Group C7: APOL6, IDO1, CXCL9, GBP5, GBP1, GBP4, CXCL11, CXCL10, LAP3, STAT1, WARS1, SAMHD1;
Group C8: ZBP1, OASL, EPSTI1, IL15RA, USP30-AS1, BATF2, ETV7, PSMB10, RTP4, CARD16;
Group C9: GZMB, GZMH, GNLY, CD8B, CTSW, CST7, NKG7, GZMA, PRF1, CD247, SLA2, PDCD1, CD7, LAG3;
Group C10: HNRNPA1P21, FOXP3, CCR8, CXCL13, AIM2, IL2RA, ICOS, CTLA4, TNFRSF9, IL21R;
Group C11: BTN3A3, BTN3A1, TAP2, NLRC5, HLA-F, PSMB8, PSMB9, TAP1, HCP5, UBE2L6, PSME2, IRF1;
Group C12: C19orf38, IGFLR1, LINC01943, RAB33A, SLC2A6, IFI30, LILRB3, IL23A, PSME2P2, ITGAE, STAC3;
Group C13: FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1;
Group C14: ADH1B, C7, CCL14, SELP, ACKR1, MMRN1, ITM2A, AQP1, ABI3BP, P2RY12;
Group C15: MPRIP, KIF13B, FYCO1, SPTLC2, ADGRA3, RBFOX2; Group C16: ITGB4, KRT17, KRT16, KRT14, KRT5, DSG3, COL17A1;
Group C17: TMEM119, PODN, SVEP1, LAMA2, COL14A1, FGF7, OGN, PRELP, ELN, MFAP4, SSC5D, PTGDS, CHRDL1;
Group C18: ITGBL1, ASPN, PDGFRB, HTRA1, HEG1;
Group C19: ZCCHC24, SGCD, SRPX, APOD, SHC4, MIA, IL17D, LRRN4CL, BOC, PDZRN3, SFRP1;
Group C20: TCF7L1, CACNA1G, SPEG, COL2A1, CRISPLD1, PIANP, NACAD, EFNB3, PCYT1B, RGMA, GLI2, PCDH19.

In some embodiments, such a gene set may include at least one gene from each of Group C3, Group C4, Group C5, Group C7, Group C9, Group C10, Group C11, Group C12, Group C13, Group C14, Group C15, Group C16, Group C17, and Group C20 and more than one gene from Group C1, Group C2, Group C6, Group C8, Group C18, and Group C19.

### Example 8D: Exemplary gene sets

In some embodiments, a gene set for use in accordance with the present disclosure includes at least one gene from the following group:
Group D1: ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MID1, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, ZCCHC24.

In some embodiments, a gene set for use in accordance with the present disclosure includes fewer than all of the genes in Group D1; in some such embodiments, a gene set for use in accordance with the present disclosure includes fewer than or equal to 100, 95, 90, 85, 80, 75, 70, 65, 60, 55, 50, 45, 40, 35, 30, 29, 28, 27 or fewer genes from Group D1.

In some embodiments, a gene set according to any one of Examples 8A-C includes one or more genes from Group D1.

### Example 9: IO Scoring of Bladder Cancer Samples

The present Example confirms that IO scores determined in accordance with the present disclosure can indicate potential response to immunomodulation therapy for various tumor types, including e.g., bladder cancer.

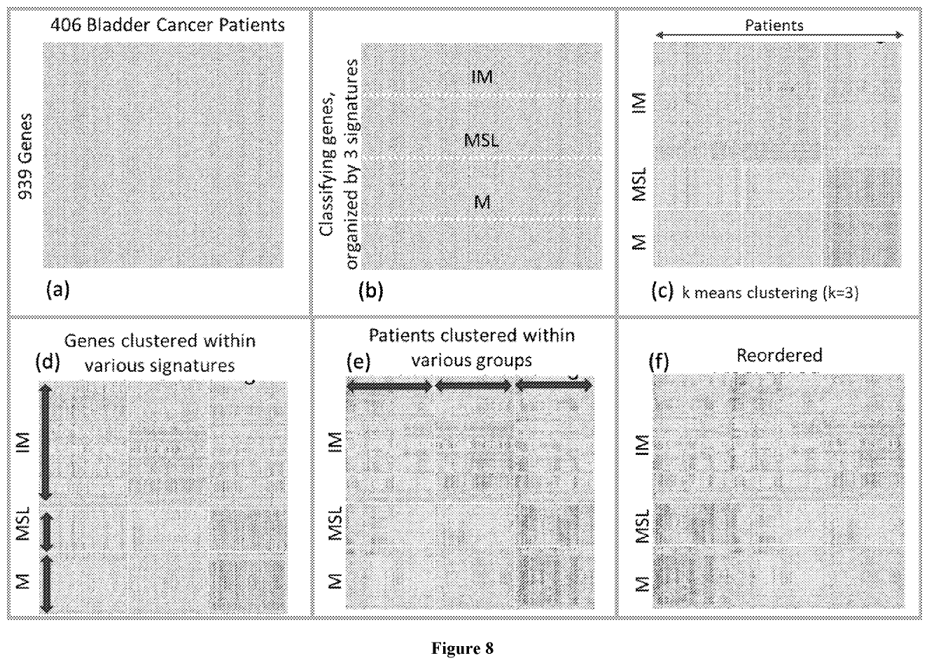

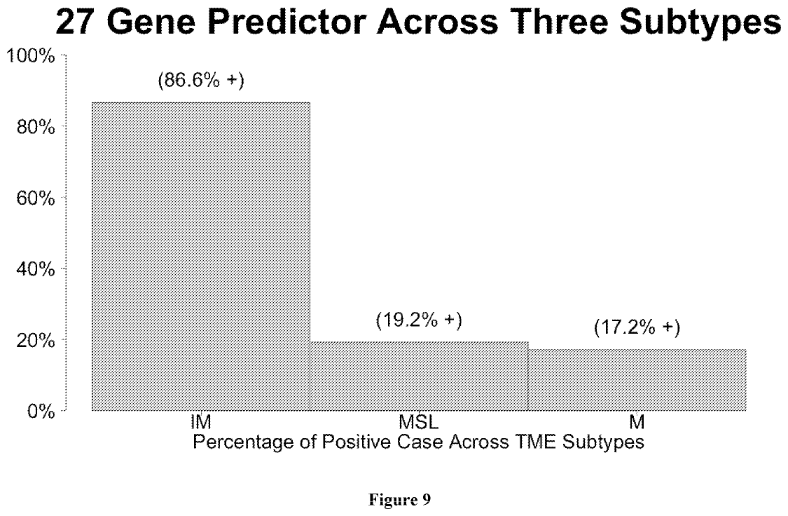

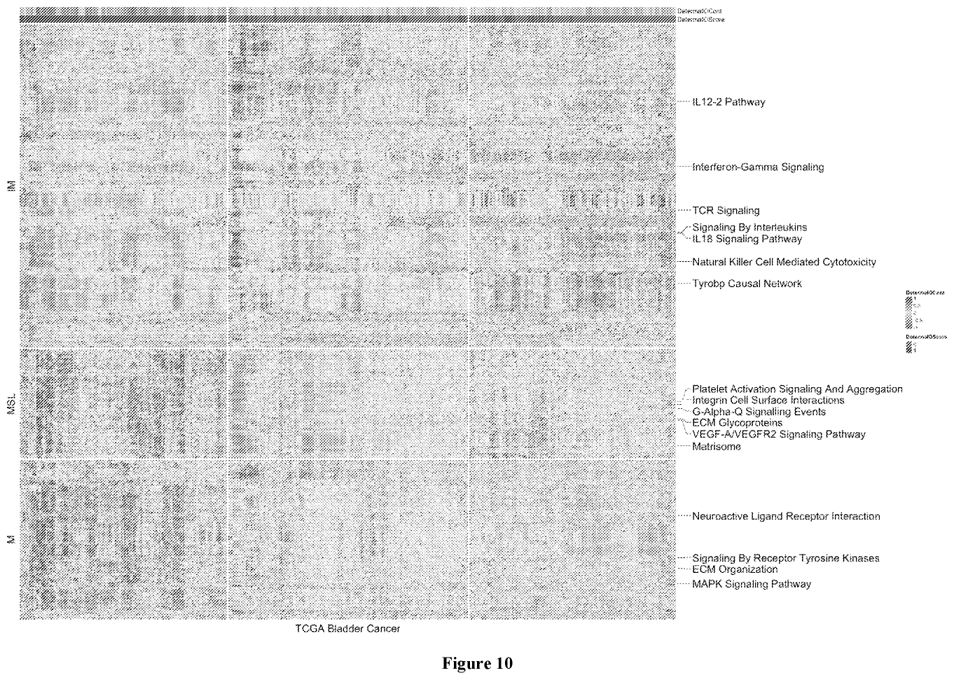

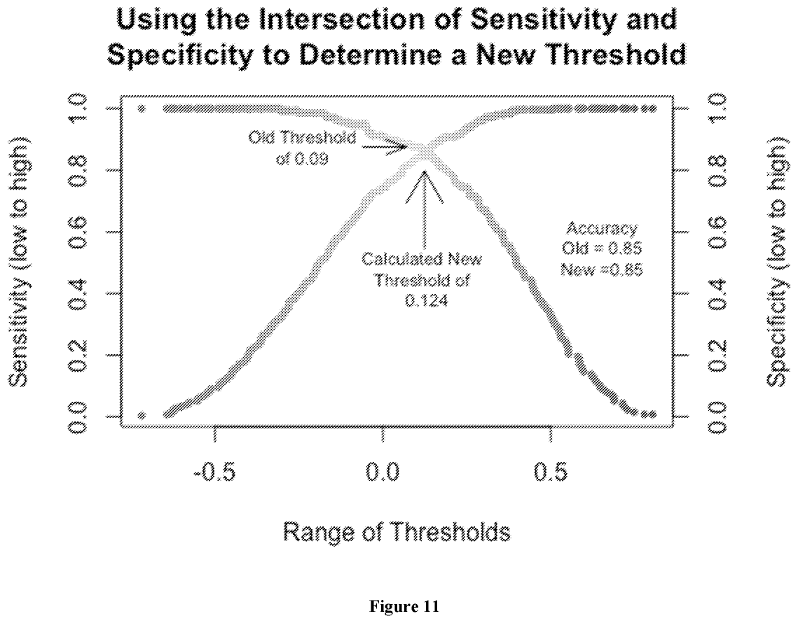

Gene expression data for 1188 breast cancer samples were downloaded and compared against an established molecular classifier (Ring et al. 2016), which selected the top 3000 genes correlated with IM, MSL, and M signatures for TNBC. The 3000 gene set was generated through assessment of the Ring et al. IM, MSL, M signatures (previously identified in TNBC) for two additional tumor types (lung adenocarcinoma and lung squamous cell carcinoma). The gene lists from all three gene expression datasets were compared and 939 genes were selected as being classifiers for IM, MSL, M based on their presence in all three gene lists. Gene expression data for 406 bladder cancer patients were downloaded and assessed using the 27-gene immuno-oncology algorithm described herein. Expression of these 939 genes were then plotted in a heatmap, clustered by signature type and patient group (FIG. 8). The 27-gene immuno-oncology algorithm IO binary score was overlaid on the heatmap, displaying an association with an IM, immunologically “hot” classification (FIG. 9, FIG. 10). These data confirm that a positive score result from the 27-gene immuno-oncology algorithm does associate with genes known to have a high, potentially active, immune function.

Furthermore, hierarchical gene clustering confirms that variations of the particular 27-gene set (e.g., including one or more changes represented in exemplary gene sets provided herein) are useful as described herein, including specifically in assessments of bladder cancer.

Hierarchical clustering of the resulting gene expression data (See, Ward, 1963, which is incorporated herein by reference in its entirety) was used to identify genes that clustered together, or metagenes, within these heatmaps. In particular, metagenes containing one or more of the 27 genes assessed as part of the immuno-oncology algorithm were evaluated. Within this subset of thirteen metagenes, a total of 198 genes were identified that could potentially be selected as alternative genes for use in the 27-gene immuno-oncology algorithm. Additionally, gene set enrichment analysis (See, Subramanian 2005, incorporated herein by reference in its entirety) of metagenes identified certain associated cellular pathways that might be of interest for assessment of tumor samples (FIG. 10). In some embodiments, these pathways may be associated with one or more genes from the 27 gene set associated with the 27-gene immuno-oncology algorithm disclosed herein (e.g., one or more of the 27 genes or their gene products may participate in the pathways). Alternatively or additionally, in some embodiments, these pathways may be associated with a specific IO score (e.g., a positive or negative score). Thus, teachings provided herein may permit selection of alternative gene sets to the 27 gene set explicitly described herein, for example including a reasonably comparable number of genes (e.g., about 10 to about 20, about 20 to about 30, about 30 to about 40, about 40 to about 50, etc.), that achieve useful tumor classification (e.g., define an IO score that discriminates) as described herein. In some embodiments, such sets may include one or more of the 27 genes of the exemplified 27 gene set, optionally in combination with one or more genes that participate in these pathways, which may be the same as or different from other genes in the exemplified 27 gene set.

Among other things, experiments confirmed that the 27-gene immuno-oncology algorithm scoring threshold, which is used as a cutoff for designating a tumor score as “positive” or “negative”, was sufficiently accurate for use in other tumor types, e.g., bladder cancer (FIG. 11). A new threshold was calculated based upon the intersection of sensitivity and specificity within bladder patient data (Habibzadeh 2016, incorporated herein by reference in its entirety) and found to have identical accuracy as compared to a previously established threshold. Therefore the original threshold was maintained for IO scoring. Thus, the present disclosure confirms, among other things, that the 27 gene set defines useful IO thresholds in a variety of cancers and, furthermore that such thresholds provide comparable accuracy, and/or are otherwise reasonably comparable (e.g., are within a range of about 0.1+/−0.02).

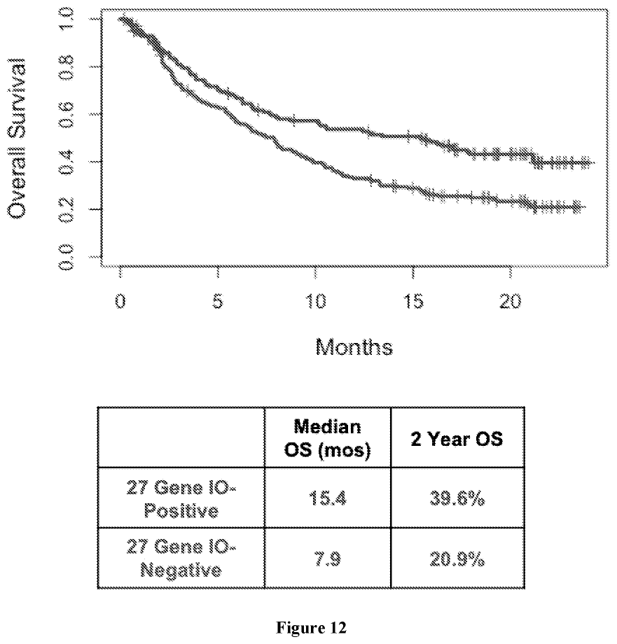

The 27-gene immuno-oncology algorithm of the present disclosure was also applied to data for a clinical cohort of bladder cancer patients treated with an immune checkpoint inhibitor (atezolizumab) in the IMVigor210 trial. Among other things, it was determined that the 27-gene immuno-oncology algorithm was able to provide a prediction of overall survival rates within the trial, based upon corresponding IO scores (FIG. 12).

### Example 10: IO Scoring of Renal Cancer Samples

The present Example confirms that IO scores determined in accordance with the present disclosure can indicate potential response to immunomodulation therapy for various tumor types, including, e.g., renal cancer.

Gene expression data for 403 clear cell kidney cancer and 203 papilloma kidney cancer patients were assessed using the 27-gene immuno-oncology algorithm described herein. Result IO scores were plotted against the 939 genes described in Example 9 above to produce heatmaps, which were clustered by signature type (IM, M, MSL) and patient group. These data confirm that a positive score result from the 27-gene immuno-oncology algorithm does associate with genes known to have a high, potentially active, immune function in certain kidney cancers.

Further experiments analyzed RNAseq data from a group of 43 renal cell carcinoma (RCC) patients that had been treated with an immuno-oncology therapy and monitored for one-year progression free survival (PFS). Patient data was assessed using the 27-gene immuno-oncology algorithm and it was found that patients with a positive IO score had significantly better one-year PFS compared to those with a negative IO score. These results confirm that the 27-gene immuno-oncology algorithm of the present disclosure has a strong correlation with response to ICI therapy in renal cancer and further support applicability of the algorithm in multiple cancer types.

### Example 11: Assessment of Data from Alternative Biological Vectors

The present Example, among other things, demonstrates that classifications provided herein can be correlated with data from alternative biological vectors (e.g., data re miRNA expression, methylation status, protein expression level, protein modification status, etc.) so that, in various embodiments, one or more different types of biological data may be utilized for and/or included in assessments of subjects and/or their immune statuses and/or responsiveness to therapy.

For example, as described herein, for a given set of patient samples for which gene expression data is obtained and IM, MSL and M centroids are assessed as described herein, matched data sets are collected along one or more alternative biological vector(s). These matched data sets can then be mapped to the gene expression centroids, which act as a reference to reveal components indicative or reflective of IM, MSL, and M features. In some embodiments, information obtained from matched data sets can be used to inform selection of one or more therapies (e.g., ICI therapy). In some embodiments, information obtained from matched data sets can be used to inform selection of combination therapies (e.g., additional therapy in combination with ICI therapy). In some embodiments, information obtained from matched data sets can be used to inform selection of one or more alternative therapies (e.g., a therapy other than ICI therapy). Thus, the present disclosure demonstrates that miRNA expression, rather than or in addition to, gene expression patterns of selected gene sets as described here, can be utilized to select and/or monitor patients for responsiveness to therapies and/or for particular characteristics of or changes in immune status.

### Example 12: Assessment of Data from miRNA Targeting Exemplary Gene Sets

The present Example, among other things, demonstrates that classifications provided herein can be correlated with pre-miRNA expression data and may be utilized for and/or included in assessments of subjects and/or their immune statuses and/or responsiveness to therapy.

A list of pre-miRNAs targeting at least one gene within the 101-gene signature was generated using a miRNA target prediction database, miRDB (http://mirdb.org/). These pre-miRNAs were then independently mapped to the IM, M, and MSL centroids (e.g., a pre-miRNA mapping to the IM centroid would be classified as having an IM signature). Classified pre-miRNAs were then assessed to determine, for example, whether an pre-miRNA was classified under a different signature than one or more of its corresponding target genes.

In some embodiments, a tumor sample (e.g., obtained through liquid biopsy, tissue biopsy, etc.) may be assessed to determine expression level of one or more classified pre-miRNAs as described herein. In some embodiments, treatments targeting or inhibiting miRNAs (e.g., pre-miRNAs, mature miRNAs, combinations thereof) classified under a different subtype as compared to one or more target genes could produce a shift in mRNA levels for said one or more target genes, resulting in changes in tumor signatures and/or IO scoring. For example, in some embodiments, treatment targeting or inhibiting miRNA (e.g., pre-miRNA, mature miRNA, combinations thereof) classified as one subtype (e.g., M, MSL) targeting a gene classified as a different subtype (e.g., IM) could produce an increase in overall IM signature for a tumor and result in an increased IO score. In some embodiments, treatment targeting or inhibiting miRNA (e.g., pre-miRNA, mature miRNA, combinations thereof) classified as one subtype (e.g., IM) targeting a gene classified as a different subtype (e.g., M, MSL) could produce an increase in overall M or MSL signature for a tumor and result in a decreased IO score. In some embodiments, information obtained from miRNA (e.g., pre-miRNA, mature miRNA, combinations thereof) assessment can be used to inform treatment decisions—e.g., selection and/or modification of therapy. In some particular embodiments, information obtained from miRNA (e.g., pre-miRNA, mature miRNA, combinations thereof) assessment can be used to inform selection and/or modification of combination therapies (e.g., additional therapy in combination with ICI therapy). In some embodiments, information obtained from matched data sets can be used to inform selection and/or modification of therapies, and in particular of combination therapies (e.g., additional therapy in combination with ICI therapy) based upon changes in IO scoring. In some embodiments, treatment targeting or inhibiting miRNA (e.g., pre-miRNA, mature miRNA, combinations thereof) could be combined with one or more therapies (e.g., chemotherapy, ICI, etc.) based upon changes in IO scoring.

### Example 13: Development of Scoring Method Based Upon miRNA Expression

The present Example, among other things, demonstrates that methods and technologies described herein may be adapted for development of a immuno-oncology algorithm to measure miRNA levels in the TME and generate an associated score predicting or otherwise characterizing response to immunomodulation therapy treatment.

Gene and pre-miRNA expression datasets for eight cancers were downloaded from the genomic data commons (GDC) portal (https://portal.gdc.cancer.gov/) using the Genomic Data Commons package in R. Assessed cancers included Bladder Urothelial Carcinoma (BLCA), Breast invasive carcinoma (BRCA), Colon Adenocarcinoma (COAD), Lung Adenocarcinoma (LUAD), Lung squamous cell carcinoma (LUSC), Ovarian serous cystadenocarcinoma (OV), Stomach adenocarcinoma (STAD) and Head and Neck squamous cell carcinoma (HNSC). Subtype (IM, M, MSL) was calculated as previously described for each sample in the datasets. STAD (n=407) and HNSC (n=545) were categorized as true test sets and OV (n=397) was used as a validation cohort. Remaining cancer datasets (BLCA, BRCA, COAD, LUAD and LUSC) were then split, with 5% of each dataset reserved to create a small validation set (n=137) and 95% concatenated to create the training set (n=2557).

With the training set established, calculations were performed to generate the spearman rho correlation coefficient between expression of pre-miRNA across all samples and subtype (IM, M, MSL), yielding three spearman rho values. Spearman rho values with a p value of <. 05 were removed from the set. Three lists of pre-miRNAs were generated containing 75 pre-miRNAs with the highest spearman rho values in a corresponding subtype (e.g., 75 pre-miRNAs for each of IM, M, and MSL). A total of 179 unique pre-miRNAs (Table 14) were obtained from this assessment, with 46 miRNAs overlapping between lists for M and MSL. Intersection of all three phenotypes was considered an empty set.

TABLE 14
Selected pre-miRNAs with highest spearman
rho coefficient in IM, M, and MSL types.
miRNA

| | | |
|---|---|---|
| hsa-mir-6720 | hsa-mir-5586 | hsa-mir-101-1 |
| hsa-let-7g | hsa-mir-30a | hsa-mir-655 |
| hsa-mir-4491 | hsa-mir-616 | hsa-mir-483 |
| hsa-mir-146b | hsa-mir-222 | hsa-mir-100 |
| hsa-mir-3682 | hsa-mir-18a | hsa-mir-19a |
| hsa-mir-223 | hsa-mir-495 | hsa-mir-488 |
| hsa-mir-490 | hsa-mir-29b-2 | hsa-mir-146a |
| hsa-mir-7-3 | hsa-mir-27b | hsa-mir-378c |
| hsa-mir-148a | hsa-mir-942 | hsa-mir-16-2 |
| hsa-mir-3926-2 | hsa-let-7a-1 | hsa-mir-181c |
| hsa-mir-502 | hsa-mir-4453 | hsa-mir-4780 |
| hsa-mir-202 | hsa-mir-576 | hsa-mir-4740 |
| hsa-mir-6507 | hsa-mir-1296 | hsa-mir-7-2 |
| hsa-mir-485 | hsa-mir-30e | hsa-mir-5694 |
| hsa-mir-29c | hsa-mir-199a-2 | hsa-mir-497 |
| hsa-mir-4772 | hsa-mir-6891 | hsa-mir-1301 |
| hsa-mir-32 | hsa-mir-505 | hsa-mir-433 |
| hsa-mir-625 | hsa-mir-579 | hsa-mir-139 |
| hsa-mir-590 | hsa-mir-7702 | hsa-mir-3677 |
| hsa-mir-494 | hsa-mir-3194 | hsa-mir-379 |
| hsa-let-7c | hsa-mir-5002 | hsa-mir-3150b |
| hsa-mir-10a | hsa-mir-577 | hsa-mir-101-2 |
| hsa-mir-127 | hsa-mir-1247 | hsa-mir-432 |
| hsa-mir-147b | hsa-mir-125b-1 | hsa-mir-186 |
| hsa-mir-143 | hsa-mir-381 | hsa-mir-501 |
| hsa-mir-3940 | hsa-let-7a-3 | hsa-mir-218-1 |
| hsa-mir-409 | hsa-mir-3136 | hsa-mir-4524a |
| hsa-mir-4736 | hsa-mir-376c | hsa-mir-766 |
| hsa-mir-496 | hsa-mir-133b | hsa-mir-301a |
| hsa-mir-340 | hsa-mir-3926-1 | hsa-mir-493 |
| hsa-mir-382 | hsa-mir-643 | hsa-let-7b |
| hsa-mir-585 | hsa-mir-150 | hsa-mir-3922 |
| hsa-mir-299 | hsa-mir-133a-1 | hsa-mir-5571 |
| hsa-mir-181d | hsa-mir-7-1 | hsa-mir-3074 |
| hsa-mir-377 | hsa-mir-511 | hsa-mir-6788 |
| hsa-mir-380 | hsa-mir-214 | hsa-mir-129-2 |
| hsa-mir-218-2 | hsa-mir-758 | hsa-mir-129-1 |
| hsa-mir-376a-1 | hsa-mir-199a-1 | hsa-mir-3690-1 |
| hsa-mir-1258 | hsa-mir-28 | hsa-mir-7846 |
| hsa-mir-454 | hsa-mir-431 | hsa-let-7a-2 |
| hsa-mir-1-1 | hsa-mir-337 | hsa-mir-665 |
| hsa-mir-889 | hsa-mir-5690 | hsa-mir-140 |
| hsa-mir-195 | hsa-mir-142 | hsa-mir-29b-1 |
| hsa-mir-216a | hsa-mir-370 | hsa-mir-15b |
| hsa-mir-16-1 | hsa-mir-204 | hsa-mir-383 |
| hsa-mir-5683 | hsa-mir-5685 | hsa-mir-145 |
| hsa-mir-369 | hsa-mir-329-1 | hsa-mir-541 |
| hsa-mir-192 | hsa-mir-4434 | hsa-mir-342 |
| hsa-let-7e | hsa-mir-155 | hsa-let-7i |
| hsa-mir-548s | hsa-mir-10b | hsa-mir-487a |
| hsa-mir-3613 | hsa-mir-411 | hsa-mir-4999 |
| hsa-mir-605 | hsa-mir-3622a | hsa-mir-133a-2 |
| hsa-mir-135a-2 | hsa-mir-3614 | hsa-mir-23b |
| hsa-mir-199b | hsa-mir-5691 | hsa-mir-410 |
| hsa-mir-148b | hsa-mir-487b | hsa-mir-6718 |
| hsa-mir-6510 | hsa-mir-29a | hsa-mir-217 |
| hsa-mir-656 | hsa-mir-628 | hsa-mir-99a |
| hsa-mir-134 | hsa-mir-1-2 | hsa-mir-654 |
| hsa-mir-4677 | hsa-mir-215 | hsa-mir-6716 |
| hsa-mir-125b-2 | hsa-mir-330 | |

Interquartile range (IQR) for expression was assessed for each of these 179 pre-miRNAs to determine which pre-miRNAs had a robust range of expression values. Pre-miRNAs with an IQR greater than 1 were selected for additional analysis, resulting in130 unique pre-miRNAs total, which are outlined in Table 15.

Next, selected pre-miRNAs were evaluated for presence of corresponding mature miRNA in real-world plasma samples. Data from GSE73002 of the NCBI gene expression omnibus (https://www.ncbi.nlm.nih.gov/geo/) were downloaded and this dataset (containing serum miRNA expression levels for breast cancer detection) was assessed for presence of selected mature miRNAs. A total of 119 unique pre-miRNAs were identified in the dataset.

TABLE 15
Selected pre-miRNAs with IQR > 1.
miRNA

| | | |
|---|---|---|
| hsa-mir-6720 | hsa-mir-5586 | hsa-mir-146a |
| hsa-mir-146b | hsa-mir-30a | hsa-mir-378c |
| hsa-mir-223 | hsa-mir-616 | hsa-mir-16-2 |
| hsa-mir-7-3 | hsa-mir-222 | hsa-mir-181c |
| hsa-mir-148a | hsa-mir-18a | hsa-mir-7-2 |
| hsa-mir-502 | hsa-mir-495 | hsa-mir-497 |
| hsa-mir-485 | hsa-mir-29b-2 | hsa-mir-1301 |
| hsa-mir-29c | hsa-mir-27b | hsa-mir-139 |
| hsa-mir-4772 | hsa-mir-942 | hsa-mir-3677 |
| hsa-mir-32 | hsa-mir-576 | hsa-mir-379 |
| hsa-mir-625 | hsa-mir-1296 | hsa-mir-3150b |
| hsa-mir-590 | hsa-mir-199a-2 | hsa-mir-101-2 |
| hsa-mir-494 | hsa-mir-505 | hsa-mir-432 |
| hsa-let-7c | hsa-mir-7702 | hsa-mir-501 |
| hsa-mir-10a | hsa-mir-577 | hsa-mir-218-1 |
| hsa-mir-127 | hsa-mir-1247 | hsa-mir-766 |
| hsa-mir-147b | hsa-mir-125b-1 | hsa-mir-301a |
| hsa-mir-143 | hsa-mir-381 | hsa-mir-493 |
| hsa-mir-3940 | hsa-mir-376c | hsa-let-7b |
| hsa-mir-409 | hsa-mir-133b | hsa-mir-3074 |
| hsa-mir-496 | hsa-mir-150 | hsa-mir-129-2 |
| hsa-mir-340 | hsa-mir-133a-1 | hsa-mir-129-1 |
| hsa-mir-382 | hsa-mir-7-1 | hsa-mir-3690-1 |
| hsa-mir-299 | hsa-mir-511 | hsa-mir-140 |
| hsa-mir-181d | hsa-mir-214 | hsa-mir-29b-1 |
| hsa-mir-377 | hsa-mir-758 | hsa-mir-15b |
| hsa-mir-380 | hsa-mir-199a-1 | hsa-mir-145 |
| hsa-mir-218-2 | hsa-mir-431 | hsa-mir-342 |
| hsa-mir-1258 | hsa-mir-337 | hsa-mir-4999 |
| hsa-mir-454 | hsa-mir-142 | hsa-mir-133a-2 |
| hsa-mir-1-1 | hsa-mir-370 | hsa-mir-23b |
| hsa-mir-889 | hsa-mir-204 | hsa-mir-410 |
| hsa-mir-195 | hsa-mir-155 | hsa-mir-6718 |
| hsa-mir-16-1 | hsa-mir-10b | hsa-mir-217 |
| hsa-mir-5683 | hsa-mir-411 | hsa-mir-99a |
| hsa-mir-369 | hsa-mir-3614 | hsa-mir-654 |
| hsa-mir-192 | hsa-mir-487b | |
| hsa-let-7e | hsa-mir-29a | |
| hsa-mir-3613 | hsa-mir-628 | |
| hsa-mir-135a-2 | hsa-mir-1-2 | |
| hsa-mir-199b | hsa-mir-215 | |
| hsa-mir-148b | hsa-mir-330 | |
| hsa-mir-6510 | hsa-mir-101-1 | |
| hsa-mir-656 | hsa-mir-655 | |
| hsa-mir-134 | hsa-mir-483 | |
| hsa-mir-4677 | hsa-mir-100 | |
| hsa-mir-125b-2 | hsa-mir-19a | |

Next, pre-miRNA interactions with potential target genes were mapped through use of miRDB (http://mirdb.org/), a miRNA target prediction database. A total of 113 pre-miRNAs were identified as potentially having at least one gene target in the 101-gene signature listing (Table 16) and 73 pre-miRNAs were identified as potentially having at least one gene target in the DetermaIO gene set (Table 17). These pre-miRNAs were also assigned a probability of interaction with each gene. Further analysis produced a list of pre-miRNAs with a target in either the 101 gene signature (Table 18) or the DetermaIO gene set (Table 19) having a probability of at least 70%.

TABLE 16
pre-miRNAs with predicted target
in 101 gene signature list.
pre-miRNA

| | | |
|---|---|---|
| hsa-mir-379 | hsa-mir-18a | hsa-mir-140 |
| hsa-let-7c | hsa-mir-15b | hsa-mir-340 |
| hsa-mir-889 | hsa-mir-628 | hsa-mir-576 |
| hsa-mir-493 | hsa-mir-7702 | hsa-mir-501 |
| hsa-mir-758 | hsa-mir-625 | hsa-mir-377 |
| hsa-let-7e | hsa-mir-218-2 | hsa-mir-299 |
| hsa-mir-29b-2 | hsa-mir-3940 | hsa-mir-27b |
| hsa-mir-4999 | hsa-mir-139 | hsa-mir-16-1 |
| hsa-mir-766 | hsa-mir-410 | hsa-mir-616 |
| hsa-mir-155 | hsa-mir-148a | hsa-mir-655 |
| hsa-mir-942 | hsa-mir-378c | hsa-mir-497 |
| hsa-mir-5683 | hsa-mir-129-1 | hsa-mir-494 |
| hsa-mir-29a | hsa-mir-3613 | hsa-mir-495 |
| hsa-mir-431 | hsa-mir-150 | hsa-mir-142 |
| hsa-mir-656 | hsa-mir-125b-2 | hsa-mir-1301 |
| hsa-mir-3614 | hsa-mir-370 | hsa-mir-6510 |
| hsa-mir-4677 | hsa-mir-10b | hsa-mir-204 |
| hsa-mir-143 | hsa-mir-145 | hsa-mir-369 |
| hsa-mir-337 | hsa-mir-502 | hsa-mir-3677 |
| hsa-mir-3074 | hsa-mir-133b | hsa-mir-496 |
| hsa-mir-100 | hsa-mir-4772 | hsa-mir-218-1 |
| hsa-mir-214 | hsa-mir-195 | hsa-mir-222 |
| hsa-mir-215 | hsa-mir-199b | hsa-mir-5586 |
| hsa-mir-181d | hsa-mir-483 | hsa-mir-382 |
| hsa-mir-134 | hsa-mir-454 | hsa-mir-148b |
| hsa-mir-511 | hsa-mir-147b | hsa-mir-192 |
| hsa-mir-146a | hsa-mir-381 | hsa-mir-577 |
| hsa-mir-217 | hsa-mir-485 | hsa-mir-127 |
| hsa-mir-181c | hsa-mir-146b | hsa-mir-19a |
| hsa-mir-3150b | hsa-mir-29c | hsa-mir-10a |
| hsa-mir-411 | hsa-mir-7-1 | hsa-mir-342 |
| hsa-mir-16-2 | hsa-mir-301a | hsa-mir-590 |
| hsa-mir-654 | hsa-mir-129-2 | hsa-mir-432 |
| hsa-mir-380 | hsa-mir-1296 | hsa-mir-32 |
| hsa-mir-409 | hsa-let-7b | hsa-mir-223 |
| hsa-mir-30a | hsa-mir-505 | hsa-mir-330 |
| hsa-mir-23b | hsa-mir-487b | hsa-mir-6720 |

TABLE 17
pre-miRNAs with predicted target in DetermaIO gene list.

| | Gene | Probability | |
|---|---|---|---|
| | Accession | of | Gene |
| miRNA | Number | Targeting | Name |
| hsa-mir-146b | NM_001278736 | 81.6941 | CCL5 |
| hsa-mir-223 | NM_004863 | 81.1901 | SPTLC2 |
| hsa-mir-148a | NM_001844 | 73.4678 | COL2A1 |
| hsa-mir-485 | NM_001171581 | 83.1959 | ITM2A |
| hsa-mir-29c | NM_001844 | 97.45356 | COL2A1 |
| hsa-mir-4772 | NM_002775 | 78.3221 | HTRA1 |
| hsa-mir-4772 | NM_004863 | 56.5861 | SPTLC2 |
| hsa-mir-32 | NM_001453 | 56.3943 | FOXC1 |
| hsa-mir-32 | NM_004863 | 58.89778 | SPTLC2 |
| hsa-mir-32 | NM_003012 | 54.10264 | SFRP1 |
| hsa-mir-32 | NM_004419 | 91.93814 | DUSP5 |
| hsa-mir-32 | NM_001193335 | 84.3651 | ASPN |
| hsa-mir-625 | NM_003012 | 50.5249 | SFRP1 |
| hsa-mir-590 | NM_004863 | 58.70663 | SPTLC2 |
| hsa-mir-590 | NM_001302123 | 58.31867 | CXCL11 |
| hsa-mir-494 | NM_001190943 | 65.46408 | TNFSF10 |
| hsa-let-7c | NM_001171581 | 89.4822 | ITM2A |
| hsa-mir-496 | NM_001171581 | 53.6328 | ITM2A |
| hsa-mir-340 | NM_001190943 | 56.8947 | TNFSF10 |
| hsa-mir-299 | NM_001077654 | 93.57209 | TNFAIP8 |
| hsa-mir-181d | NM_004419 | 70.5801 | DUSP5 |
| hsa-mir-181d | NM_001193335 | 73.0576 | ASPN |
| hsa-mir-181d | NM_001190943 | 51.8133 | TNFSF10 |
| hsa-mir-181d | NM_001193335 | 85.7785 | ASPN |
| hsa-mir-380 | NM_001453 | 84.3932 | FOXC1 |
| hsa-mir-889 | NM_001844 | 95.91891 | COL2A1 |
| hsa-mir-16-1 | NM_004419 | 74.7026 | DUSP5 |
| hsa-mir-16-1 | NM_003012 | 72.3666 | SFRP1 |
| hsa-mir-5683 | NM_002164 | 58.29101 | IDO1 |
| hsa-mir-369 | NM_001193335 | 51.33138 | ASPN |
| hsa-mir-192 | NM_003012 | 54.4194 | SFRP1 |
| hsa-mir-192 | NM_001193335 | 77.7057 | ASPN |
| hsa-mir-3613 | NM_004419 | 87.96533 | DUSP5 |
| hsa-mir-3613 | NM_001171581 | 68.55283 | ITM2A |
| hsa-mir-3613 | NM_001171581 | 81.5659 | ITM2A |
| hsa-mir-199b | NM_001302123 | 58.422 | CXCL11 |
| hsa-mir-199b | NM_004419 | 55.1577 | DUSP5 |
| hsa-mir-199b | NM_004863 | 50.36683 | SPTLC2 |
| hsa-mir-148b | NM_001844 | 73.4678 | COL2A1 |
| hsa-mir-6510 | NM_001190943 | 72.2871 | TNFSF10 |
| hsa-mir-134 | NM_001844 | 77.4781 | COL2A1 |
| hsa-mir-4677 | NM_004863 | 88.3559 | SPTLC2 |
| hsa-mir-4677 | NM_003012 | 73.45515 | SFRP1 |
| hsa-mir-4677 | NM_003012 | 58.41983 | SFRP1 |
| hsa-mir-4677 | NM_001844 | 64.4672 | COL2A1 |
| hsa-mir-125b-2 | NM_004863 | 81.1744 | SPTLC2 |
| hsa-mir-5586 | NM_001844 | 86.4851 | COL2A1 |
| hsa-mir-616 | NM_001193335 | 83.66219 | ASPN |
| hsa-mir-222 | NM_001302123 | 54.7001 | CXCL11 |
| hsa-mir-18a | NM_001077654 | 78.6758 | TNFAIP8 |
| hsa-mir-18a | NM_001844 | 51.9634 | COL2A1 |
| hsa-mir-495 | NM_001453 | 65.72426 | FOXC1 |
| hsa-mir-495 | NM_002775 | 67.9268 | HTRA1 |
| hsa-mir-495 | NM_001171581 | 96.44696 | ITM2A |
| hsa-mir-29b-2 | NM_001077654 | 68.4902 | TNFAIP8 |
| hsa-mir-27b | NM_004419 | 73.7791 | DUSP5 |
| hsa-mir-27b | NM_004863 | 82.47013 | SPTLC2 |
| hsa-mir-27b | NM_003012 | 93.27801 | SFRP1 |
| hsa-mir-942 | NM_003012 | 68.1522 | SFRP1 |
| hsa-mir-942 | NM_004863 | 79.63713 | SPTLC2 |
| hsa-mir-576 | NM_003012 | 76.7581 | SFRP1 |
| hsa-mir-376c | NM_001077654 | 65.51542 | TNFAIP8 |
| hsa-mir-133b | NM_001302123 | 50 | CXCL11 |
| hsa-mir-133b | NM_004419 | 76.9457 | DUSP5 |
| hsa-mir-133b | NM_001453 | 79.5863 | FOXC1 |
| hsa-mir-150 | NM_004863 | 55.13409 | SPTLC2 |
| hsa-mir-7-1 | NM_003012 | 64.3331 | SFRP1 |
| hsa-mir-511 | NM_001647 | 83.1836 | APOD |
| hsa-mir-511 | NM_004863 | 84.70739 | SPTLC2 |
| hsa-mir-511 | NM_001077654 | 64.9614 | TNFAIP8 |
| hsa-mir-511 | NM_002775 | 62.2474 | HTRA1 |
| hsa-mir-214 | NM_001190943 | 51.6121 | TNFSF10 |
| hsa-mir-758 | NM_001278736 | 64.9068 | CCL5 |
| hsa-mir-758 | NM_001190943 | 64.0171 | TNFSF10 |
| hsa-mir-758 | NM_004863 | 55.0982 | SPTLC2 |
| hsa-mir-142 | NM_004863 | 51.4403 | SPTLC2 |
| hsa-mir-204 | NM_001453 | 97.1037 | FOXC1 |
| hsa-mir-204 | NM_016584 | 62.8694 | IL23A |
| hsa-mir-155 | NM_001077654 | 64.1595 | TNFAIP8 |
| hsa-mir-155 | NM_002164 | 82.2192 | IDO1 |
| hsa-mir-10b | NM_003012 | 73.7255 | SFRP1 |
| hsa-mir-3614 | NM_001077654 | 87.36709 | TNFAIP8 |
| hsa-mir-487b | NM_001302123 | 89.14869 | CXCL11 |
| hsa-mir-29a | NM_001193335 | 56.2208 | ASPN |
| hsa-mir-29a | NM_001844 | 97.45356 | COL2A1 |
| hsa-mir-628 | NM_002775 | 62.15602 | HTRA1 |
| hsa-mir-215 | NM_001077654 | 61.3624 | TNFAIP8 |
| hsa-mir-215 | NM_004863 | 51.96964 | SPTLC2 |
| hsa-mir-330 | NM_001453 | 55.4929 | FOXC1 |
| hsa-mir-330 | NM_003012 | 70.2497 | SFRP1 |
| hsa-mir-655 | NM_001453 | 81.2044 | FOXC1 |
| hsa-mir-655 | NM_003012 | 51.4532 | SFRP1 |
| hsa-mir-19a | NM_002775 | 76.157 | HTRA1 |
| hsa-mir-19a | NM_003012 | 61.70509 | SFRP1 |
| hsa-mir-146a | NM_001278736 | 81.6941 | CCL5 |
| hsa-mir-146a | NM_001453 | 99.13475 | FOXC1 |
| hsa-mir-146a | NM_004863 | 59.43829 | SPTLC2 |
| hsa-mir-181c | NM_004419 | 70.5801 | DUSP5 |
| hsa-mir-181c | NM_001193335 | 73.0576 | ASPN |
| hsa-mir-181c | NM_001190943 | 51.8133 | TNFSF10 |
| hsa-mir-7-2 | NM_003012 | 64.3331 | SFRP1 |
| hsa-mir-1301 | NM_001647 | 53.771 | APOD |
| hsa-mir-379 | NM_001302123 | 83.8024 | CXCL11 |
| hsa-mir-432 | NM_001190943 | 53.58751 | TNFSF10 |
| hsa-mir-501 | NM_001453 | 80.2884 | FOXC1 |
| hsa-mir-218-1 | NM_004863 | 67.60088 | SPTLC2 |
| hsa-mir-301a | NM_001453 | 72.43312 | FOXC1 |
| hsa-mir-301a | NM_001171581 | 70.7526 | ITM2A |
| hsa-mir-493 | NM_001077654 | 80.6789 | TNFAIP8 |
| hsa-mir-3074 | NM_001453 | 65.20962 | FOXC1 |
| hsa-mir-3074 | NM_001844 | 52.13298 | COL2A1 |
| hsa-mir-140 | NM_001302123 | 86.3204 | CXCL11 |
| hsa-mir-140 | NM_001171581 | 54.9455 | ITM2A |
| hsa-mir-145 | NM_002775 | 81.3835 | HTRA1 |
| hsa-mir-145 | NM_004863 | 67.75261 | SPTLC2 |
| hsa-mir-23b | NM_004863 | 58.86111 | SPTLC2 |
| hsa-mir-23b | NM_001453 | 88.2522 | FOXC1 |
| hsa-mir-23b | NM_004419 | 78.7952 | DUSP5 |
| hsa-mir-23b | NM_001193335 | 61.9589 | ASPN |
| hsa-mir-410 | NM_001190943 | 65.46408 | TNFSF10 |
| hsa-mir-217 | NM_001077654 | 94.65965 | TNFAIP8 |
| hsa-mir-217 | NM_004863 | 66.77802 | SPTLC2 |
| hsa-mir-217 | NM_002775 | 50.98073 | HTRA1 |
| hsa-mir-217 | NM_001171581 | 53.7001 | ITM2A |
| hsa-mir-654 | NM_001077654 | 58.5766 | TNFAIP8 |

TABLE 18
pre-miRNAs with predicted target in 101 gene
signature list having at least 70% probability.
pre-miRNA

| | | |
|---|---|---|
| hsa-mir-379 | hsa-mir-18a | hsa-mir-501 |
| hsa-let-7c | hsa-mir-15b | hsa-mir-377 |
| hsa-mir-889 | hsa-mir-625 | hsa-mir-299 |
| hsa-mir-493 | hsa-mir-218-2 | hsa-mir-27b |
| hsa-mir-758 | hsa-mir-3940 | hsa-mir-16-1 |
| hsa-let-7e | hsa-mir-148a | hsa-mir-616 |
| hsa-mir-29b-2 | hsa-mir-3613 | hsa-mir-655 |
| hsa-mir-4999 | hsa-mir-125b-2 | hsa-mir-497 |
| hsa-mir-766 | hsa-mir-370 | hsa-mir-494 |
| hsa-mir-155 | hsa-mir-10b | hsa-mir-495 |
| hsa-mir-942 | hsa-mir-145 | hsa-mir-1301 |
| hsa-mir-29a | hsa-mir-502 | hsa-mir-6510 |
| hsa-mir-431 | hsa-mir-133b | hsa-mir-204 |
| hsa-mir-656 | hsa-mir-4772 | hsa-mir-3677 |
| hsa-mir-3614 | hsa-mir-195 | hsa-mir-496 |
| hsa-mir-4677 | hsa-mir-199b | hsa-mir-218-1 |
| hsa-mir-143 | hsa-mir-483 | hsa-mir-222 |
| hsa-mir-3074 | hsa-mir-454 | hsa-mir-5586 |
| hsa-mir-214 | hsa-mir-147b | hsa-mir-382 |
| hsa-mir-215 | hsa-mir-381 | hsa-mir-148b |
| hsa-mir-181d | hsa-mir-485 | hsa-mir-192 |
| hsa-mir-134 | hsa-mir-146b | hsa-mir-577 |
| hsa-mir-511 | hsa-mir-29c | hsa-mir-127 |
| hsa-mir-146a | hsa-mir-7-1 | hsa-mir-19a |
| hsa-mir-217 | hsa-mir-301a | hsa-mir-590 |
| hsa-mir-181c | hsa-let-7b | hsa-mir-432 |
| hsa-mir-411 | hsa-mir-140 | hsa-mir-32 |
| hsa-mir-16-2 | hsa-mir-487b | hsa-mir-223 |
| hsa-mir-380 | hsa-mir-505 | hsa-mir-330 |
| hsa-mir-30a | hsa-mir-340 | hsa-mir-6720 |
| hsa-mir-23b | hsa-mir-576 | hsa-mir-7-2 |
| hsa-mir-376c | | |

TABLE 19
pre-miRNAs with predicted target in DetermaIO
gene list having at least 70% probability.

| | Gene Accession | Probability of | |
|---|---|---|---|
| miRNA | Number | Targeting | Gene Name |
| hsa-mir-146b | NM_001278736 | 81.6941 | CCL5 |
| hsa-mir-223 | NM_004863 | 81.1901 | SPTLC2 |
| hsa-mir-148a | NM_001844 | 73.4678 | COL2A1 |
| hsa-mir-485 | NM_001171581 | 83.1959 | ITM2A |
| hsa-mir-29c | NM_001844 | 97.45356 | COL2A1 |
| hsa-mir-4772 | NM_002775 | 78.3221 | HTRA1 |
| hsa-mir-32 | NM_004419 | 91.93814 | DUSP5 |
| hsa-mir-32 | NM_001193335 | 84.3651 | ASPN |
| hsa-let-7c | NM_001171581 | 89.4822 | ITM2A |
| hsa-mir-299 | NM_001077654 | 93.57209 | TNFAIP8 |
| hsa-mir-181d | NM_004419 | 70.5801 | DUSP5 |
| hsa-mir-181d | NM_001193335 | 73.0576 | ASPN |
| hsa-mir-181d | NM_001193335 | 85.7785 | ASPN |
| hsa-mir-380 | NM_001453 | 84.3932 | FOXC1 |
| hsa-mir-889 | NM_001844 | 95.91891 | COL2A1 |
| hsa-mir-16-1 | NM_004419 | 74.7026 | DUSP5 |
| hsa-mir-16-1 | NM_003012 | 72.3666 | SFRP1 |
| hsa-mir-192 | NM_001193335 | 77.7057 | ASPN |
| hsa-mir-3613 | NM_004419 | 87.96533 | DUSP5 |
| hsa-mir-3613 | NM_001171581 | 81.5659 | ITM2A |
| hsa-mir-148b | NM_001844 | 73.4678 | COL2A1 |
| hsa-mir-6510 | NM_001190943 | 72.2871 | TNFSF10 |
| hsa-mir-134 | NM_001844 | 77.4781 | COL2A1 |
| hsa-mir-4677 | NM_004863 | 88.3559 | SPTLC2 |
| hsa-mir-4677 | NM_003012 | 73.45515 | SFRP1 |
| hsa-mir-125b-2 | NM_004863 | 81.1744 | SPTLC2 |
| hsa-mir-5586 | NM_001844 | 86.4851 | COL2A1 |
| hsa-mir-616 | NM_001193335 | 83.66219 | ASPN |
| hsa-mir-18a | NM_001077654 | 78.6758 | TNFAIP8 |
| hsa-mir-495 | NM_001171581 | 96.44696 | ITM2A |
| hsa-mir-27b | NM_004419 | 73.7791 | DUSP5 |
| hsa-mir-27b | NM_004863 | 82.47013 | SPTLC2 |
| hsa-mir-27b | NM_003012 | 93.27801 | SFRP1 |
| hsa-mir-942 | NM_004863 | 79.63713 | SPTLC2 |
| hsa-mir-576 | NM_003012 | 76.7581 | SFRP1 |
| hsa-mir-133b | NM_004419 | 76.9457 | DUSP5 |
| hsa-mir-133b | NM_001453 | 79.5863 | FOXC1 |
| hsa-mir-511 | NM_001647 | 83.1836 | APOD |
| hsa-mir-511 | NM_004863 | 84.70739 | SPTLC2 |
| hsa-mir-204 | NM_001453 | 97.1037 | FOXC1 |
| hsa-mir-155 | NM_002164 | 82.2192 | IDO1 |
| hsa-mir-10b | NM_003012 | 73.7255 | SFRP1 |
| hsa-mir-3614 | NM_001077654 | 87.36709 | TNFAIP8 |
| hsa-mir-487b | NM_001302123 | 89.14869 | CXCL11 |
| hsa-mir-29a | NM_001844 | 97.45356 | COL2A1 |
| hsa-mir-330 | NM_003012 | 70.2497 | SFRP1 |
| hsa-mir-655 | NM_001453 | 81.2044 | FOXC1 |
| hsa-mir-19a | NM_002775 | 76.157 | HTRA1 |
| hsa-mir-146a | NM_001278736 | 81.6941 | CCL5 |
| hsa-mir-146a | NM_001453 | 99.13475 | FOXC1 |
| hsa-mir-181c | NM_004419 | 70.5801 | DUSP5 |
| hsa-mir-181c | NM_001193335 | 73.0576 | ASPN |
| hsa-mir-379 | NM_001302123 | 83.8024 | CXCL11 |
| hsa-mir-501 | NM_001453 | 80.2884 | FOXC1 |
| hsa-mir-301a | NM_001453 | 72.43312 | FOXC1 |
| hsa-mir-301a | NM_001171581 | 70.7526 | ITM2A |
| hsa-mir-493 | NM_001077654 | 80.6789 | TNFAIP8 |
| hsa-mir-140 | NM_001302123 | 86.3204 | CXCL11 |
| hsa-mir-145 | NM_002775 | 81.3835 | HTRA1 |
| hsa-mir-23b | NM_001453 | 88.2522 | FOXC1 |
| hsa-mir-23b | NM_004419 | 78.7952 | DUSP5 |
| hsa-mir-217 | NM_001077654 | 94.65965 | TNFAIP8 |

Further analysis determined that certain pre-miRNAs could interact with multiple genes (e.g., multiple genes from the 101 gene signature and/or DetermaIO gene list). A total of 16 pre-miRNAs were found to potentially interact with only a single gene in the 101 gene signature list (Table 20) with a probability of at least 70%. A total of 42 pre-miRNAs were found to potentially interact with only a single gene in the DetermaIO gene list with a probability of at least 70% (Table 21).

TABLE 20
pre-miRNAs with a single predicted target in 101
gene signature list having at least 70% probability.
pre-miRNA

hsa-mir-3677
hsa-mir-342
hsa-mir-3150b
hsa-let-7e
hsa-mir-378c
hsa-mir-5586
hsa-mir-3940
hsa-mir-483
hsa-mir-454
hsa-mir-100
hsa-mir-139
hsa-mir-409
hsa-mir-7702
hsa-mir-10a
hsa-mir-337
hsa-mir-431

TABLE 21A
pre-miRNAs with a single predicted target in DetermaIO
gene list having at least 70% probability.

| | Gene Accession | Probability of | Gene |
|---|---|---|---|
| miRNA | Number | Targeting | Name |
| hsa-mir-146b | NM_001278736 | 81.6941 | CCL5 |
| hsa-mir-223 | NM_004863 | 81.1901 | SPTLC2 |
| hsa-mir-148a | NM_001844 | 73.4678 | COL2A1 |
| hsa-mir-485 | NM_001171581 | 83.1959 | ITM2A |
| hsa-mir-29c | NM_001844 | 97.45356 | COL2A1 |
| hsa-mir-625 | NM_003012 | 50.5249 | SFRP1 |
| hsa-mir-494 | NM_001190943 | 65.46408 | TNFSF10 |
| hsa-let-7c | NM_001171581 | 89.4822 | ITM2A |
| hsa-mir-496 | NM_001171581 | 53.6328 | ITM2A |
| hsa-mir-340 | NM_001190943 | 56.8947 | TNFSF10 |
| hsa-mir-299 | NM_001077654 | 93.57209 | TNFAIP8 |
| hsa-mir-380 | NM_001453 | 84.3932 | FOXC1 |
| hsa-mir-889 | NM_001844 | 95.91891 | COL2A1 |
| hsa-mir-5683 | NM_002164 | 58.29101 | IDO1 |
| hsa-mir-369 | NM_001193335 | 51.33138 | ASPN |
| hsa-mir-148b | NM_001844 | 73.4678 | COL2A1 |
| hsa-mir-6510 | NM_001190943 | 72.2871 | TNFSF10 |
| hsa-mir-134 | NM_001844 | 77.4781 | COL2A1 |
| hsa-mir-125b-2 | NM_004863 | 81.1744 | SPTLC2 |
| hsa-mir-5586 | NM_001844 | 86.4851 | COL2A1 |
| hsa-mir-616 | NM_001193335 | 83.66219 | ASPN |
| hsa-mir-222 | NM_001302123 | 54.7001 | CXCL11 |
| hsa-mir-29b-2 | NM_001077654 | 68.4902 | TNFAIP8 |
| hsa-mir-576 | NM_003012 | 76.7581 | SFRP1 |
| hsa-mir-376c | NM_001077654 | 65.51542 | TNFAIP8 |
| hsa-mir-150 | NM_004863 | 55.13409 | SPTLC2 |
| hsa-mir-7-1 | NM_003012 | 64.3331 | SFRP1 |
| hsa-mir-214 | NM_001190943 | 51.6121 | TNFSF10 |
| hsa-mir-142 | NM_004863 | 51.4403 | SPTLC2 |
| hsa-mir-10b | NM_003012 | 73.7255 | SFRP1 |
| hsa-mir-3614 | NM_001077654 | 87.36709 | TNFAIP8 |
| hsa-mir-487b | NM_001302123 | 89.14869 | CXCL11 |
| hsa-mir-628 | NM_002775 | 62.15602 | HTRA1 |
| hsa-mir-7-2 | NM_003012 | 64.3331 | SFRP1 |
| hsa-mir-1301 | NM_001647 | 53.771 | APOD |
| hsa-mir-379 | NM_001302123 | 83.8024 | CXCL11 |
| hsa-mir-432 | NM_001190943 | 53.58751 | TNFSF10 |
| hsa-mir-501 | NM_001453 | 80.2884 | FOXC1 |
| hsa-mir-218-1 | NM_004863 | 67.60088 | SPTLC2 |
| hsa-mir-493 | NM_001077654 | 80.6789 | TNFAIP8 |
| hsa-mir-410 | NM_001190943 | 65.46408 | TNFSF10 |
| hsa-mir-654 | NM_001077654 | 58.5766 | TNFAIP8 |

Methods and technologies described above were further applied to analysis of mature miRNA. Gene and mature miRNA expression datasets for five cancers were downloaded from the genomic data commons (GDC) portal (https://portal.gdc.cancer.gov/) using the Genomic Data Commons package in R. Assessed cancers included Bladder Urothelial Carcinoma (BLCA), Breast invasive carcinoma (BRCA), Colon Adenocarcinoma (COAD), Lung Adenocarcinoma (LUAD), and Lung squamous cell carcinoma (LUSC). Subtype was calculated as previously described for each sample in the datasets. Cancer types (BLCA, BRCA, COAD, LUAD and LUSC) were then split so that 5% of each dataset was reserved to create a small validation set (n-97) and 95% were concatenated to create the training set (n=1875).

With the training set established, calculations were performed to generate the spearman rho correlation coefficient, between the expression of mature miRNA across all samples and the subtype (IM, M, MSL), yielding three spearman rho values. Spearman rho values with a p value of <. 05 were removed from the set. Three lists of mature miRNAs were generated containing 75 mature miRNAs with the highest spearman rho values in a corresponding subtype (e.g., 75 mature miRNAs for each of IM, M, and MSL). A total of 151 unique mature miRNAs were obtained from this assessment (Table 21B).

TABLE 21B
Selected mature miRNAs with highest spearman rho coefficient in IM, M, and MSL types.
Mature miRNA

| | | | | | |
|---|---|---|---|---|---|
| hsa-miR-375 | hsa-miR-143-5p | hsa-miR-500a-3p | hsa-miR-532-3p | hsa-miR-199a-5p | hsa-miR-130a-3p |
| hsa-miR-146b-3p | hsa-miR-625-3p | hsa-miR-193a-5p | hsa-miR-148a-5p | hsa-miR-532-5p | hsa-miR-26a-5p |
| hsa-miR-150-5p | hsa-miR-93-5p | hsa-miR-30e-5p | hsa-miR-21-3p | hsa-miR-10b-5p | hsa-let-7g-5p |
| hsa-miR-222-3p | hsa-miR-99b-3p | hsa-miR-10a-5p | hsa-miR-148a-3p | hsa-let-7b-5p | hsa-miR-199b-3p |
| hsa-miR-143-3p | hsa-miR-9-5p | hsa-miR-200b-3p | hsa-miR-140-3p | hsa-miR-181b-5p | hsa-miR-425-5p |
| hsa-miR-221-3p | hsa-let-7c-5p | hsa-miR-127-3p | hsa-miR-155-5p | hsa-miR-214-5p | hsa-miR-379-5p |
| hsa-miR-199b-5p | hsa-let-7a-5p | hsa-miR-181a-2-3p | hsa-miR-30c-2-3p | hsa-miR-181c-5p | |
| hsa-miR-30a-5p | hsa-miR-197-3p | hsa-miR-584-5p | hsa-miR-744-5p | hsa-miR-185-5p | |
| hsa-miR-27b-3p | hsa-miR-16-5p | hsa-miR-186-5p | hsa-miR-29a-3p | hsa-miR-15a-5p | |
| hsa-miR-27b-5p | hsa-miR-345-5p | hsa-miR-92a-3p | hsa-miR-141-5p | hsa-miR-30a-3p | |
| hsa-miR-339-5p | hsa-miR-423-5p | hsa-miR-22-5p | hsa-miR-23b-3p | hsa-let-7e-3p | |
| hsa-miR-126-5p | hsa-miR-100-5p | hsa-miR-574-3p | hsa-let-7i-5p | hsa-let-7i-3p | |
| hsa-miR-497-5p | hsa-miR-589-5p | hsa-miR-342-3p | hsa-miR-192-5p | hsa-miR-330-5p | |
| hsa-miR-141-3p | hsa-miR-28-5p | hsa-miR-146b-5p | hsa-miR-203a-3p | hsa-miR-93-3p | |
| hsa-miR-181a-3p | hsa-miR-103a-3p | hsa-miR-152-3p | hsa-miR-423-3p | hsa-let-7d-5p | |
| hsa-miR-576-5p | hsa-miR-139-5p | hsa-miR-26b-5p | hsa-miR-17-3p | hsa-miR-1307-3p | |
| hsa-miR-23a-3p | hsa-miR-106b-5p | hsa-miR-335-3p | hsa-miR-191-5p | hsa-miR-1301-3p | |
| hsa-miR-145-3p | hsa-miR-101-3p | hsa-miR-148b-3p | hsa-miR-744-3p | hsa-miR-378a-3p | |
| hsa-miR-337-3p | hsa-miR-28-3p | hsa-miR-629-5p | hsa-miR-145-5p | hsa-miR-361-5p | |
| hsa-miR-151a-5p | hsa-miR-874-3p | hsa-let-7b-3p | hsa-miR-132-3p | hsa-miR-374a-3p | |
| hsa-miR-125a-5p | hsa-miR-199a-3p | hsa-miR-127-5p | hsa-miR-25-3p | hsa-miR-200c-3p | |
| hsa-miR-29c-5p | hsa-miR-652-3p | hsa-miR-339-3p | hsa-miR-24-3p | hsa-miR-1976 | |
| hsa-miR-29b-3p | hsa-miR-425-3p | hsa-miR-193b-3p | hsa-miR-107 | hsa-miR-484 | |
| hsa-miR-128-3p | hsa-miR-181c-3p | hsa-miR-15b-5p | hsa-miR-542-3p | hsa-miR-378a-5p | |
| hsa-miR-34a-5p | hsa-miR-126-3p | hsa-miR-424-5p | hsa-miR-331-3p | hsa-miR-125b-5p | |
| hsa-miR-664a-3p | hsa-miR-223-3p | hsa-miR-766-3p | hsa-miR-99b-5p | hsa-miR-194-5p | |
| hsa-miR-451a | hsa-miR-21-5p | hsa-miR-29c-3p | hsa-miR-142-3p | hsa-miR-99a-5p | |
| hsa-miR-505-3p | hsa-miR-106b-3p | hsa-miR-30e-3p | hsa-miR-20a-5p | hsa-miR-92b-3p | |
| hsa-let-7e-5p | hsa-miR-134-5p | hsa-miR-17-5p | hsa-miR-361-3p | hsa-miR-210-3p | |

Interquartile range (IQR) for expression was assessed for these 151 mature miRNAs to determine which mature miRNAs had a robust range of expression values. Mature miRNAs with an IQR greater than 1 were selected for additional analysis, resulting in 134 unique mature miRNAs total.

TABLE 21C
Selected mature miRNAs with IQR >1.
Mature miRNA

| | | | | |
|---|---|---|---|---|
| hsa-miR-375 | hsa-miR-625-3p | hsa-miR-181a-2-3p | hsa-miR-141-5p | hsa-miR-330-5p |
| hsa-miR-146b-3p | hsa-miR-93-5p | hsa-miR-584-5p | hsa-miR-23b-3p | hsa-miR-93-3p |
| hsa-miR-150-5p | hsa-miR-99b-3p | hsa-miR-92a-3p | hsa-miR-192-5p | hsa-miR-1307-3p |
| hsa-miR-222-3p | hsa-miR-9-5p | hsa-miR-22-5p | hsa-miR-203a-3p | hsa-miR-1301-3p |
| hsa-miR-143-3p | hsa-let-7c-5p | hsa-miR-574-3p | hsa-miR-423-3p | hsa-miR-378a-3p |
| hsa-miR-221-3p | hsa-miR-197-3p | hsa-miR-342-3p | hsa-miR-17-3p | hsa-miR-200c-3p |
| hsa-miR-199b-5p | hsa-miR-16-5p | hsa-miR-146b-5p | hsa-miR-191-5p | hsa-miR-1976 |
| hsa-miR-30a-5p | hsa-miR-345-5p | hsa-miR-152-3p | hsa-miR-744-3p | hsa-miR-484 |
| hsa-miR-27b-3p | hsa-miR-423-5p | hsa-miR-26b-5p | hsa-miR-145-5p | hsa-miR-378a-5p |
| hsa-miR-27b-5p | hsa-miR-100-5p | hsa-miR-335-3p | hsa-miR-24-3p | hsa-miR-125b-5p |
| hsa-miR-339-5p | hsa-miR-589-5p | hsa-miR-148b-3p | hsa-miR-107 | hsa-miR-194-5p |
| hsa-miR-126-5p | hsa-miR-139-5p | hsa-miR-629-5p | hsa-miR-542-3p | hsa-miR-99a-5p |
| hsa-miR-497-5p | hsa-miR-106b-5p | hsa-let-7b-3p | hsa-miR-331-3p | hsa-miR-92b-3p |
| hsa-miR-141-3p | hsa-miR-101-3p | hsa-miR-127-5p | hsa-miR-99b-5p | hsa-miR-210-3p |
| hsa-miR-181a-3p | hsa-miR-874-3p | hsa-miR-339-3p | hsa-miR-142-3p | hsa-miR-130a-3p |
| hsa-miR-576-5p | hsa-miR-199a-3p | hsa-miR-193b-3p | hsa-miR-20a-5p | hsa-miR-199b-3p |
| hsa-miR-145-3p | hsa-miR-652-3p | hsa-miR-15b-5p | hsa-miR-361-3p | hsa-miR-425-5p |
| hsa-miR-337-3p | hsa-miR-425-3p | hsa-miR-424-5p | hsa-miR-199a-5p | hsa-miR-379-5p |
| hsa-miR-151a-5p | hsa-miR-181c-3p | hsa-miR-766-3p | hsa-miR-532-5p | |
| hsa-miR-125a-5p | hsa-miR-126-3p | hsa-miR-29c-3p | hsa-miR-10b-5p | |
| hsa-miR-29c-5p | hsa-miR-223-3p | hsa-miR-17-5p | hsa-let-7b-5p | |
| hsa-miR-29b-3p | hsa-miR-106b-3p | hsa-miR-532-3p | hsa-miR-181b-5p | |
| hsa-miR-128-3p | hsa-miR-134-5p | hsa-miR-148a-5p | hsa-miR-214-5p | |
| hsa-miR-34a-5p | hsa-miR-500a-3p | hsa-miR-21-3p | hsa-miR-181c-5p | |
| hsa-miR-664a-3p | hsa-miR-193a-5p | hsa-miR-148a-3p | hsa-miR-185-5p | |
| hsa-miR-451a | hsa-miR-30e-5p | hsa-miR-155-5p | hsa-miR-15a-5p | |
| hsa-miR-505-3p | hsa-miR-10a-5p | hsa-miR-30c-2-3p | hsa-miR-30a-3p | |
| hsa-let-7e-5p | hsa-miR-200b-3p | hsa-miR-744-5p | hsa-let-7e-3p | |
| hsa-miR-143-5p | hsa-miR-127-3p | hsa-miR-29a-3p | hsa-let-7i-3p | |

Next, selected mature miRNAs were evaluated in real-world plasma samples.

Data from GSE73002 from the NCBI gene expression omnibus (https://www.ncbi.nlm.nih.gov/geo/) were downloaded and this dataset (containing serum miRNA expression levels for breast cancer detection) were assessed for presence of selected mature miRNAs. A total of 131 unique miRNAs were identified in the dataset.

Among other things, the present Example demonstrates that available miRNA expression data may be utilized, and, in some embodiments, combined with the 101 gene signature to produce a model to classify miRNAs (e.g., pre-miRNAs, mature miRNAs, combinations thereof) into a particular signature type (e.g., IM, M, MSL). In some embodiments, this model may be used to assess a sample of interest (e.g., tumor sample from a subject, blood and/or plasma sample from a subject) for miRNA (e.g., pre-miRNA, mature miRNA, combinations thereof) expression levels in order to assess IM, M, and MSL signature levels. In some embodiments, assessment of gene signature levels could be leveraged to create a blood-based, miRNA-specific scoring system to inform selection and/or modification of therapy (e.g., ICI therapy, chemotherapy, etc.).

### Example 14: Assessment of Gene Mutation Data

The present Example, among other things, demonstrates that classifications provided herein can be correlated with gene mutation and interaction data and may be utilized for and/or included in assessments of subjects and/or their immune statuses and/or responsiveness to therapy.

A dataset comprising gene mutation and interaction data (e.g., amino acid changes, affected protein, mutation type, participant information) was downloaded from IntAct database (https://www.ebi.ac.uk/intact/home). The dataset was analyzed for interactions with one or more genes within the gene list described in Example 15, resulting in a list of 4285 gene mutations of interest. These gene mutations were further analyzed to identify any potential interactions with other genes within the 939 gene list, producing a list of 309 interactions of interest.

Next, identified gene mutations with interactions were assessed to determine whether certain mutations could result in altered interactions, e.g., whether a wild-type protein would interact with a protein with a signature of IM, M, or MSL, and an identified mutation would interact with a protein from a different signature of IM, M, or MSL. Gene mutations were mapped to IM, M, or MSL subtypes based upon clustering analysis for each tumor sample, wherein a gene was within a certain Euclidean distance for each subtype. Gene mutations were assigned to a single subtype if (i) they clustered with one subtype based upon data from 11 or more different tumor tissue types or if (ii) they clustered with one subtype based upon data from 10 different tumor tissue types and clustered with a different subtype in 6 or less different tumor tissue types. Gene mutations were assigned to multiple subtypes if (i) they clustered with one subtype based upon data from 10 tumor tissue types and clustered with a different subtype based upon data from 7 or 8 different tumor tissue types, (ii) they clustered with one subtype based upon data from 8 or 9 tumor tissue types and clustered with a different subtype based upon data from 7-9 different tumor tissue types. Gene mutations were not given a subtype if they clustered with one subtype based upon data from 80r 9 tumor tissue types and clustered with a different subtype based upon data from 6 or less different tumor tissue types.

After assignment of subtypes, gene mutations were filtered to identify interactions between genes (e.g., interactions of expressed mutant protein with a wild-type or mutant protein of interest) wherein a gene of one subtype interacted with a gene of another subtype. A total of 92 gene mutations of interest were identified as a result of this analysis. Gene mutations with no predicted effect were then removed, producing a list of 64 gene mutations of interest (Table 22). Mutation descriptions in Table 22 are presented in formatting as outlined in IntAct database (https://www.ebi.ac.uk/intact/download/datasets #mutations).

TABLE 22
Gene mutations interacting with one or more genes of a different subtype.

| Affected | Affected | | |
|---|---|---|---|
| protein symbol | Gene Subtypes | Feature type | Interactions |
| HTRA1 | M | mutation(MI: 0118) | [‘HTRA4:IM’] |
| LPL | M | mutation(MI: 0118) | [‘GPIHBP1:MSL’] |
| MPP1 | IM | mutation(MI: 0118) | [‘GYPC:MSL’] |
| HTRA4 | IM | mutation(MI: 0118) | [‘HTRA1:M’] |
| FGFR1 | M | mutation(MI: 0118) | [‘FGF2:MSL_M’] |
| GUCY1B1 | MSL_M | mutation(MI: 0118) | [‘GUCY1A1:MSL’] |
| GNAO1 | M | mutation(MI: 0118) | [‘GNG2:MSL’] |
| PILRA | IM | mutation(MI: 0118) | [‘SORCS1:MS_M’] |
| CCL5 | IM | mutation(MI: 0118) | [‘CXCL12:MSL’] |
| GYPC | MSL | mutation(MI: 0118) | [‘MPP1:IM’] |
| ADAM8 | IM | mutation(MI: 0118) | [‘SNX33:M’] |
| PLEK | IM | mutation(MI: 0118) | [‘CAVIN2:MSL’, ‘F13A1:MSL’] |
| PLEK | IM | mutation(MI: 0118) | [‘ACTN1:M’] |
| BTK | IM | mutation decreasing(MI: 0119) | [‘GTF21:M’] |
| FYN | MSL | mutation decreasing(MI: 0119) | [‘PRKCQ:IM’] |
| GPIHBP1 | MSL | mutation decreasing(MI: 0119) | [‘LPL:M’] |
| BTK | IM | mutation decreasing(MI: 0119) | [‘SH3BP5:MSL_M’] |
| IGFBP5 | M | mutation decreasing(MI: 0119) | [‘IGF1:MSL’] |
| CFH | MSL | mutation decreasing(MI: 0119) | [‘C3:IM_MSL’] |
| LAT | IM | mutation decreasing(MI: 0119) | [‘PLCG1:M’] |
| NOD2 | IM | mutation decreasing strength(MI: 1133) | [‘VIM:MSL’] |
| LILRB2 | IM | mutation decreasing strength(MI: 1133) | [‘ANGPTL2:M’] |
| APOE | IM | mutation decreasing strength(MI: 1133) | [‘LRP1:M’] |
| PRKN | M | mutation decreasing strength(MI: 1133) | [‘UBE2L6:IM’] |
| LRP1 | M | mutation decreasing strength(MI: 1133) | [‘APOE:IM’] |
| NTRK3 | MSL | mutation decreasing strength(MI: 1133) | [‘DOK6:M’] |
| LRP1 | M | mutation decreasing strength(MI: 1133) | [‘APOE:IM’] |
| THEMIS | IM | mutation decreasing strength(MI: 1133) | [‘PLCG1:M’] |
| FOXP3 | IM | mutation disrupting(MI: 0573) | [‘RBPMS:M’] |
| WARS1 | IM | mutation disrupting(MI: 0573) | [‘CDH5:MSL’] |
| VCAM1 | IM | mutation disrupting(MI: 0573) | [‘SPTBN1:M’, ‘APOE:IM’, ‘TLN1:M’, |
| | | | ‘PSMA3:IM’, ‘S100A8:IM’, ‘PSMA6:IM’] |
| CFH | MSL | mutation disrupting(MI: 0573) | [‘C3:IM_MSL’] |
| SH2D1B | IM | mutation disrupting(MI: 0573) | [‘INPP5D:IM’, ‘DAPP1:IM’, ‘PECAM1:MSL’, |
| | | | ‘CD84:IM’] |
| PLCG1 | M | mutation disrupting(MI: 0573) | [‘MAP4K1:IM’] |
| FOXP3 | IM | mutation disrupting(MI: 0573) | [‘RBPMS:M’] |
| LY96 | IM | mutation disrupting(MI: 0573) | [‘TLR4:IM_MSL’] |
| VCAM1 | IM | mutation disrupting(MI: 0573) | [‘TLN1:M’, ‘VCL:M’, ‘PSMA3:IM’, ‘FERMT3 |
| | | | :IM’, ‘PSMA6:IM’] |
| VCAM1 | IM | mutation disrupting(MI: 0573) | [‘TLN1:M’] |
| VCAM1 | IM | mutation disrupting(MI: 0573) | [‘KIDINS220:M’, ‘TMPO:IM_M’, ‘TLN1:M’, |
| | | | ‘PSMA3:IM’, ‘PSMA6:IM’, ‘HIP1:M’, |
| | | | ‘FLNC:M’, ‘SPTBN1:M’] |
| HTRA1 | M | mutation disrupting rate(MI: 1129) | [‘HTRA4:IM’] |
| CTNNA3 | M | mutation disrupting strength(MI: 1128) | [‘USHBP1:MSL’] |
| LPL | M | mutation disrupting strength(MI: 1128) | [‘GPIHBP1:MSL’] |
| FAM107A | MSL | mutation disrupting strength(MI: 1128) | [‘CCDC136:M’] |
| PRKN | M | mutation disrupting strength(MI: 1128) | [‘UBE2L6:IM’] |
| BTK | IM | mutation disrupting strength(MI: 1128) | [‘MEOX2:MSL’] |
| NTRK3 | MSL | mutation disrupting strength(MI: 1128) | [‘DOK6:M’] |
| PRKN | M | mutation disrupting strength(MI: 1128) | [‘NFKBIE:IM’] |
| LILRB2 | IM | mutation disrupting strength(MI: 1128) | [‘ANGPTL2:M’] |
| ESR1 | IM_MSL | mutation disrupting strength(MI: 1128) | [‘ESR2:IM_M’] |
| PILRA | IM | mutation disrupting strength(MI: 1128) | [‘PIANP:M’] |
| NTRK3 | MSL | mutation disrupting strength(MI: 1128) | [‘NTF3:M’, ‘DOK6:M’] |
| APOE | IM | mutation disrupting strength(MI: 1128) | [‘LRP1:M’] |
| DPYSL2 | MSL_M | mutation disrupting strength(MI: 1128) | [‘DPYSL3:M’] |
| DNMT3A | M | mutation disrupting strength(MI: 1128) | [‘TCL1A:IM’] |
| GPIHBP1 | MSL | mutation disrupting strength(MI: 1128) | [‘LPL:M’] |
| PBXIP1 | M | mutation disrupting strength(MI: 1128) | [‘ESR1:IM_MSL’] |
| ESR1 | IM_MSL | mutation disrupting strength(MI: 1128) | [‘STAT1:IM’] |
| LDOC1 | M | mutation disrupting strength(MI: 1128) | [‘FAM107A:MSL’] |
| SELP | MSL | mutation increasing(MI: 0382) | [‘SELPLG:IM’] |
| LILRB2 | IM | mutation increasing strength(MI: 1132) | [‘ANGPTL2:M’] |
| KDR | MSL | mutation increasing strength(MI: 1132) | [‘ANTXR1:M’] |
| ANTXR1 | M | mutation increasing strength(MI: 1132) | [‘KDR:MSL’] |
| PILRA | IM | mutation increasing strength(MI: 1132) | [‘PIANP:M’] |
| FGFR1 | M | mutation increasing strength(MI: 1132) | [‘FGF2:MSL_M’] |

Among other things, the present Example demonstrates that gene mutations and/or interactions may be assessed to determine potential interactions with genes of interest that map to one or more of the IM, M, and MSL subtypes (e.g., genes from the 101-gene signature list, DetermaIO list, and/or larger gene sets based upon clustering with the 101-gene signature list). In some embodiments, certain gene mutations that map to one subtype (e.g., IM, M, MSL) may be identified as having potential interactions with one or more genes of interest mapping to a different subtype (e.g., IM, M, MSL). In some embodiments, such gene mutations may be used to inform treatment of a subject. For example, in some embodiments, treatment targeting or inhibiting a mutated gene having one signature (e.g., M, MSL) that interacts with a gene having a different signature (e.g., IM) could produce an increase in overall IM signature for a tumor and result in an increased IO score. In some embodiments, treatment targeting or inhibiting mutated gene having one signature (e.g., IM) that interacts with a gene having a different signature (e.g., M, MSL) could produce an increase in overall M or MSL signature for a tumor and result in a decreased IO score. In some embodiments, information obtained from gene mutation assessment can be used to inform selection and/or modification of therapy, and particularly of combination therapies (e.g., additional therapy in combination with ICI therapy). In some embodiments, information obtained from gene mutations can be used to inform selection and/or modification of therapy, and particularly of combination therapies (e.g., additional therapy in combination with ICI therapy) based upon changes in IO scoring. In some embodiments, treatment targeting or inhibiting mutated genes could be combined with one or more therapies (e.g., chemotherapy, ICI, etc.) based upon changes in IO scoring. In some embodiments, treatment targeting or inhibiting a mutated gene (e.g., via one or more of an antibody, small molecule, antibody conjugate, etc.) could be targeted to one or more regions the corresponding expressed protein (e.g., one or more regions that is/are associated with mutation).

In some embodiments, the present example demonstrates that certain gene mutations (e.g., mutation in FGFR1) may associate with the M subtype and/or may increase affinity for one or more genes in the MSL subtype (e.g., MSL). In some embodiments, increased interactions between or among genes in M and MSL subtypes may promote reduced tumor responsiveness to one or more therapies. Accordingly, in some embodiments, the present disclosure teaches that a treatment may be selected specifically targeting such gene mutations; without wishing to be bound by any particular theory, the present disclosure teaches that administering such targeted treatment (and, in some embodiments, administering such in combination with IO therapy), may provide improved subject outcome as compared to a non-targeted therapy and/or to IO therapy alone.

In some embodiments, the present example demonstrates that certain gene mutations (e.g., mutation in SELP) may associate with the MSL subtype and increase affinity for one or more genes in the IM subtype (e.g., MSL). In some embodiments, a treatment may be selected specifically targeting such gene mutations, for example in order to increase expression of genes associated with IM (e.g., potentially increasing IM subtype character of a tumor and/or DetermaIO scoring).

### Example 15: Assessment of Compound Sensitivity

The present Example, among other things, demonstrates that classifications provided herein can be correlated with compound sensitivity data and may be utilized for and/or included in assessments of subjects and/or their immune statuses and/or responsiveness to therapy.

Datasets of tumor sensitivity to various compounds as part of the PRISM screen were downloaded (https://depmap.org/portal/download/). Compounds were annotated with compound names and mechanisms of action based upon publicly available data. Analysis was restricted to cell line assays not annotated as ‘Failed’, resulting in a total of 568 cell lines and 4686 compounds. Gene expression data for a set of 19177 genes on 1389 cell lines was also acquired from the Broad Institute (https://depmap.org/portal/download/). Cell lines in the gene expression data set were split into 2/3 training and 1/3 test sets, balanced for cell lineage, using the ‘caret’ package in R version 4.1.1. Cells were classified by subtype (e.g., IM, M, MSL), as described herein using the 101 gene signature. Data was limited to cell lines that were included in the gene expression training set and were also employed in the compound sensitivity screen. This resulted in a set of 4686 compounds applied to 371 cell lines. The test set consisted of 4686 compounds applied to 188 cell lines.

Compounds were limited to those with a significant (p value <0.05) negative correlation with the IM, MSL or M subtypes, determined by classifying cell lines using the 101 gene signatures as previously herein, and then correlating the compound sensitivity scores with the subtype correlations across the training and test sets of cell lines. Only compounds with a correlation of less than −0.1 with at least one subtype correlation in the training cell set were retained for further analysis. This resulted in a set of 598 compounds, some of which were not uniformly associated with the different subtypes (Table 23A).

TABLE 23A
Compounds with at least one subtype correlation in training cell set.

| BROAD compound ID | Compound Name | Mechanism of Action | TNBCType train | DTIO Corr train | IM Corr test | MSL Corr test | M Corr test | DTIO Corr test |
|---|---|---|---|---|---|---|---|---|
| A00077618 | 8-bromo-cGMP | PKA activator | MSL | 0.074852 | −0.00708 | −0.05953 | −0.06633 | −0.017 |
| A00842753 | oleuropein | estrogen receptor agonist | MSL | −0.00213 | 0.035013 | −0.08668 | −0.08237 | 0.021715 |
| A04497688 | trilostane | 3beta-hydroxy-delta5-steroid dehydrogenase inhibitor | MSL | −0.01163 | −0.099 | −0.00526 | 0.01632 | −0.05152 |
| A09472452 | flecainide | sodium channel blocker | IM | −0.16563 | −0.0668 | 0.045385 | −0.00817 | −0.0192 |
| A10523515 | GSK429286A | rho associated kinase inhibitor | MSL | −0.00189 | 0.070284 | −0.17861 | −0.16057 | 0.102994 |
| A10773072 | glycerol-monolaurate | beta lactamase inhibitor | M | 0.048439 | 0.223193 | −0.11751 | −0.22188 | 0.143213 |
| A10977446 | carvedilol | adrenergic receptor antagonist | IM | −0.08384 | −0.04298 | −0.07456 | 0.045591 | 0.000729 |
| A11990600 | lorazepam | benzodiazepine receptor agonist | IM | −0.13184 | −0.08811 | 0.090871 | 0.097022 | −0.02916 |
| A12230535 | nutlin-3 | MDM inhibitor | M | 0.160633 | 0.06852 | 0.035363 | −0.07413 | 0.150256 |
| A12454076 | L-Hydroxyproline | | IM | −0.0814 | −0.01657 | −0.15111 | −0.01403 | −0.05682 |
| A14886633 | norgestrel | progesterone receptor agonist | M | 0.080278 | 0.057726 | 9.57E−05 | −0.14398 | 0.045486 |
| A16263897 | misonidazole | | MSL | −0.00067 | −0.1232 | −0.0539 | 0.078077 | −0.138 |
| A17009129 | isavuconazole | cytochrome P450 inhibitor | MSL | 0.080655 | 0.107481 | −0.1043 | −0.06544 | 0.043623 |
| A18992208 | lercanidipine | calcium channel blocker | IM | −0.10567 | −0.05125 | 0.056732 | 0.064398 | 0.040143 |
| A19736161 | ondansetron | serotonin receptor antagonist | IM | −0.07634 | −0.1174 | −0.01358 | 0.062424 | −0.11235 |
| A19777893 | menadione-bisulfite | vitamin K | M | 0.003549 | 0.192533 | −0.10861 | −0.20263 | 0.136223 |
| A23124853 | PF-06463922 | ALK tyrosine kinase receptor inhibitor | MSL | −0.07689 | −0.12186 | −0.05668 | −0.02078 | −0.00728 |
| A23127772 | sclareol | | MSL | 0.027899 | −0.0091 | −0.00812 | 0.006849 | −0.02449 |
| A23418262 | floctafenine | cyclooxygenase inhibitor | MSL | −0.0399 | 0.041975 | −0.04168 | −0.04815 | 0.127147 |
| A24514565 | warfarin | vitamin K antagonist | M | 0.141944 | −0.00917 | −0.02248 | −0.08567 | 0.068712 |
| A24587114 | isoetharine | adrenergic receptor agonist | IM | −0.15006 | −0.06951 | 0.0084 | −0.01333 | −0.05616 |
| A25004090 | erastin | ion channel antagonist | MSL | −0.08613 | −0.13507 | −0.07532 | 0.020114 | −0.06388 |
| A27883417 | alexidine | phosphatidylglycerophosphatase inhibitor | M | 0.209638 | 0.162958 | 0.011116 | −0.14599 | 0.100148 |
| A28970875 | puromycin | protein synthesis inhibitor | M | 0.027776 | 0.064756 | 0.012902 | −0.11839 | 0.066029 |
| A29520968 | acifran | cholesterol inhibitor | IM | −0.02068 | −0.0076 | 0.126024 | 0.002814 | −0.00246 |
| A29700858 | carbenoxolone | 11-beta hydroxysteroid dehydrogenase inhibitor | IM | −0.07689 | −0.06543 | −0.01512 | 0.00454 | −0.05769 |
| A30590053 | MR-16728 | acetylcholine release enhancer | IM | −0.16056 | −0.01298 | 0.064867 | 0.025101 | 0.043526 |
| A30886737 | florfenicol | protein synthesis inhibitor | M | 0.077868 | 0.082266 | 0.003813 | −0.06536 | 0.130946 |
| A31811760 | miltefosine | membrane integrity inhibitor | IM | −0.02111 | −0.06197 | 0.091874 | 0.016792 | −0.04486 |
| A33711280 | metixene | acetylcholine receptor antagonist | IM | −0.14865 | −0.09271 | 0.106076 | 0.113219 | −0.04991 |
| A34255068 | rolipram | phosphodiesterase inhibitor | IM | −0.13204 | −0.16867 | 0.120148 | 0.145406 | −0.11127 |
| A36057565 | bruceantin | protein synthesis inhibitor | M | −0.00997 | −0.02817 | 0.041762 | −0.01138 | −0.09134 |
| A36074203 | remacemide | glutamate receptor antagonist | IM | −0.09407 | −0.00275 | 0.012004 | −0.03609 | −0.0384 |
| A36267905 | buphenine | adrenergic receptor agonist | IM | −0.13436 | −0.07524 | 0.078744 | 0.077779 | −0.05463 |
| A40084411 | CGP-12177 | adrenergic receptor agonist | IM | −0.03736 | −0.08055 | −0.03092 | 0.023091 | −0.06572 |
| A40940854 | ibuproxam | cyclooxygenase inhibitor, prostaglandin inhibitor | M | 0.110699 | −0.02197 | 0.073601 | −0.04584 | −0.12705 |
| A41698174 | bacampicillin | bacterial cell wall synthesis inhibitor | MSL | 0.028395 | 0.044536 | −0.07656 | −0.07737 | 0.079115 |
| A42270467 | tenatoprazole | ATPase inhibitor | MSL | 0.114311 | 0.047184 | −0.05139 | 0.019855 | 0.004184 |
| A43999749 | homatropine-methylbromide | acetylcholine receptor antagonist | M | 0.050905 | 0.063393 | −0.01662 | −0.00694 | 0.016977 |
| A44181516 | flumecinol | | MSL | 0.02706 | 0.064037 | −0.09398 | −0.06491 | 0.037858 |
| A44188509 | AZD 1446 | acetylcholine receptor agonist | MSL | −0.01778 | −0.01023 | −0.09838 | −0.00226 | −0.00648 |
| A44511856 | ginsenoside-rg3 | angiogenesis inhibitor; apoptosis stimulant | M | 0.056597 | 0.007852 | −0.02955 | −0.05841 | −0.00056 |
| A44971162 | ODM-201 | androgen receptor antagonist | MSL | 0.003412 | −0.03339 | −0.13807 | 0.043792 | −0.01761 |
| A45889380 | mepacrine | cytokine production inhibitor; NFkB pathway inhibitor; | IM | −0.17027 | −0.10962 | 0.208632 | 0.183871 | −0.04932 |
| | | TP53 activator | | | | | | |
| A50675490 | carazolol | adrenergic receptor antagonist | MSL | 0.050713 | −0.1204 | −0.02083 | 0.041574 | −0.06042 |
| A51747092 | moprolol | adrenergic receptor antagonist | IM | −0.14525 | −0.07275 | −0.02538 | −0.00898 | −0.14209 |
| A52660433 | tetrindole | monoamine oxidase inhibitor | IM | −0.09692 | −0.02376 | 0.060719 | 0.06046 | −0.04907 |
| A55312468 | k-strophanthidin | ATPase inhibitor | MSL | −0.01124 | −0.06974 | −0.10988 | −0.03165 | −0.03307 |
| A55484088 | BNTX | opioid receptor antagonist | IM | −0.1416 | −0.13712 | 0.106241 | 0.093233 | −0.10848 |
| A56085258 | LGX818 | RAF inhibitor | M | 0.095349 | 0.222544 | 0.162724 | −0.23255 | 0.14417 |
| A56241705 | trimethoquinol | adrenergic receptor antagonist | MSL | −0.0983 | −0.06802 | −0.05303 | −0.00108 | −0.05537 |
| A56359832 | zileuton | leukotriene synthesis inhibitor; lipoxygenase inhibitor | MSL | −0.00728 | 0.067338 | −0.05561 | −0.06242 | 0.13867 |
| A56675431 | althiazide | diuretic | M | −0.02356 | 0.043864 | −0.10294 | −0.07072 | 0.080778 |
| A58947127 | AMG319 | PI3K inhibitor | MSL | −0.03859 | 0.079864 | −0.08814 | −0.17127 | 0.032831 |
| A59378440 | butylphthalide | potassium channel antagonist | MSL | 0.011784 | −0.03693 | −0.02003 | −0.03289 | −0.10822 |
| A59961917 | BQ-123 | endothelin receptor antagonist | MSL | 0.04394 | 0.044903 | −0.14206 | −0.13738 | 0.076412 |
| A64610707 | pranoprofen | cyclooxygenase inhibitor | M | 0.100929 | 0.061883 | −0.10549 | −0.07721 | −0.01897 |
| A64743628 | isometheptene-mucate | | IM | −0.11792 | −0.09289 | 0.117989 | 0.05313 | −0.04443 |
| A68083442 | pranidipine | calcium channel blocker | M | 0.103012 | 0.012969 | −0.01285 | −0.08946 | 0.040504 |
| A69917777 | aminopentamide | acetylcholine receptor antagonist | MSL | 0.018261 | −0.00471 | −0.07475 | −0.04959 | −0.03995 |
| A71033472 | fendiline | calcium channel blocker | IM | −0.16084 | −0.12538 | −0.04896 | 0.112397 | −0.11301 |
| A71407503 | inimur | other antifungal | IM | −0.08883 | −0.09878 | 0.031385 | 0.038736 | −0.04743 |
| A72297358 | ozolinone | diuretic | MSL | 0.043059 | −0.13218 | −0.06039 | −0.01029 | −0.09683 |
| A72401848 | gadoteridol | radiopaque medium | MSL | −0.04116 | −0.02624 | −0.00929 | −0.02135 | −0.08228 |
| A73679382 | MK-571 | leukotriene receptor antagonist | MSL | 0.024321 | −0.02535 | −0.00401 | −0.02967 | −0.00581 |
| A75479906 | rimantadine | antiviral; RNA synthesis inhibitor | IM | −0.0701 | −0.13735 | 0.124737 | 0.121882 | −0.05017 |
| A75769826 | SDM25N | opioid receptor antagonist | IM | −0.12001 | −0.16816 | −0.06407 | 0.088071 | −0.16502 |
| A75975749 | bafetinib | Bcr-Abl kinase inhibitor; LYN tyrosine kinase inhibitor | M | 0.119134 | 0.263708 | −0.01663 | −0.24548 | 0.165805 |
| A78341343 | regadenoson | adenosine receptor agonist | IM | −0.15857 | −0.08832 | −0.05759 | 0.02954 | −0.10154 |
| A78877355 | nefopam | cyclooxygenase inhibitor | IM | −0.13132 | −0.14884 | −0.12309 | 0.021929 | −0.14163 |
| A79803969 | memantine | glutamate receptor antagonist | IM | −0.03846 | −0.20725 | 0.101963 | 0.159677 | −0.1964 |
| A80213327 | NSC-23766 | Ras GTPase inhibitor | M | 0.067501 | −0.00484 | 0.133217 | −0.06657 | 0.047997 |
| A80280426 | AD-5467 | aldose reductase inhibitor | MSL | 0.018022 | −0.0506 | −0.05143 | 0.039021 | −0.04673 |
| A81370665 | BI-D1870 | ribosomal protein inhibitor | IM | −0.09163 | −0.01584 | 0.020052 | 0.010011 | 0.018544 |
| A82522119 | tibolone | androgen receptor agonist; estrogen receptor agonist; | IM | −0.11619 | −0.09026 | −0.06141 | −0.05265 | −0.05238 |
| | | progesterone receptor agonist | | | | | | |
| A84810646 | ginsenoside-rd | calcium channel blocker | MSL | −0.02011 | −0.05744 | −0.02352 | −0.00784 | −0.0392 |
| A85025557 | NCS-382 | GABA receptor antagonist | IM | −0.13426 | −0.09394 | 0.059282 | 0.105905 | −0.17053 |
| A86871940 | nicaraven | free radical scavenger | IM | −0.12278 | −0.05557 | 0.071825 | −0.00159 | 0.039544 |
| A88562598 | degarelix | gonadotropin releasing factor hormone receptor antagonist | MSL | 0.019957 | 0.000275 | −0.05421 | −0.03554 | −0.01776 |
| A92630576 | trimebutine | opioid receptor agonist | M | −0.00435 | 0.02851 | −0.03839 | −0.0841 | −0.05432 |
| A92872453 | N-acetylmannosamine | | IM | −0.0802 | −0.09969 | −0.06809 | 0.083615 | −0.05912 |
| A96456596 | FPL-55712 | leukotriene receptor antagonist | IM | −0.1264 | −0.05838 | 0.075493 | 0.133834 | −0.01746 |
| A97739905 | ketoprofen | cyclooxygenase inhibitor | MSL | 0.060339 | −0.06437 | −0.01815 | 0.051319 | 0.01612 |
| A98845662 | desvenlafaxine | serotonin norepinephrine reuptake inhibitor (SNRI) | IM | −0.16436 | −0.09107 | 0.048536 | 0.047545 | −0.04076 |
| K01039503 | sofalcone | mucus protecting agent | IM | −0.05742 | −0.05509 | 0.114824 | 0.04839 | 0.045539 |
| K01291782 | molidustat | hypoxia inducible factor inhibitor | IM | −0.12926 | −0.10307 | −0.046 | 0.151199 | −0.08823 |
| K01507359 | rifampin | RNA polymerase inhibitor | M | 0.115452 | 0.110211 | −0.11515 | −0.18692 | 0.057333 |
| K01624185 | maltobionic-acid | matrix metalloprotease inhibitor | M | 0.09859 | 0.107806 | 0.037165 | −0.08169 | 0.130029 |
| K01942991 | oxotremorine-m | acetylcholine receptor agonist | IM | −0.0669 | −0.08895 | 0.097283 | 0.059918 | −0.11284 |
| K02113016 | olaparib | PARP inhibitor | M | 0.026605 | 0.05691 | −0.10496 | −0.11335 | 0.064041 |
| K02152879 | sivelestat | elastase inhibitor | IM | −0.10576 | −0.00698 | 0.06219 | 0.007744 | 0.049544 |
| K02594908 | sulfanilamide | carbonic anhydrase inhibitor | MSL | −0.03491 | 0.054725 | −0.01366 | 0.006753 | 0.023484 |
| K02750403 | piboserod | serotonin receptor antagonist | MSL | 0.011882 | −0.02493 | −0.05998 | 0.008828 | 0.020963 |
| K02965577 | secalciferol | vitamin D receptor agonist | IM | −0.06151 | −0.12541 | −0.05502 | 0.078662 | −0.09515 |
| K03063480 | PF-477736 | CHK inhibitor | M | −0.01779 | 0.057954 | −0.10663 | −0.16439 | −0.03209 |
| K03164761 | thiazovivin | rho associated kinase inhibitor | MSL | 0.033738 | 0.039098 | −0.21347 | −0.10822 | 0.048453 |
| K03289018 | CCT137690 | Aurora kinase inhibitor | M | −0.02552 | −0.03424 | −0.02426 | −0.09289 | −0.13958 |
| K03384561 | roquinimex | angiogenesis inhibitor; tumor necrosis factor production | IM | −0.09623 | −0.00663 | 0.030006 | −0.00406 | 0.023981 |
| | | inhibitor | | | | | | |
| K03503561 | ravuconazole | sterol demethylase inhibitor | MSL | 0.098854 | 0.028167 | −0.00902 | −0.01521 | −0.03731 |
| K03739921 | hypoxanthine | PARP inhibitor | MSL | 0.021509 | 0.031275 | −0.2363 | −0.03639 | −0.01455 |
| K03765900 | XL-647 | EGFR inhibitor; VEGFR inhibitor | IM | −0.1539 | −0.18202 | 0.364371 | 0.231003 | −0.05086 |
| K04264130 | favipiravir | RNA polymerase inhibitor | IM | −0.12869 | −0.03139 | 0.094929 | −0.04351 | 0.002354 |
| K04394237 | drospirenone | mineralocorticoid receptor antagonist | M | 0.10521 | 0.118209 | 0.004094 | −0.08392 | 0.072095 |
| K04568635 | octenidine | membrane integrity inhibitor | M | 0.147947 | 0.164049 | 0.028498 | −0.21749 | 0.09423 |
| K04603573 | 1,12-Besm | polyamine biosynthesis inhibitor | M | 0.07515 | −0.00575 | −0.13217 | −0.0837 | 0.020164 |
| K04623885 | BIBR-1532 | telomerase inhibitor | MSL | −0.05255 | −0.17124 | −0.14817 | 0.140976 | −0.22597 |
| K05048137 | phytosphingosine | | IM | −0.10885 | −0.06346 | 0.017576 | 0.066885 | 0.004108 |
| K05445342 | WP1066 | STAT inhibitor | IM | −0.08426 | −0.15525 | 0.075395 | 0.129476 | −0.129 |
| K05619559 | tobramycin | bacterial 30S ribosomal subunit inhibitor | IM | −0.08252 | −0.03111 | −0.10534 | −0.017 | −0.03868 |
| K05638300 | sevoflurane | membrane integrity inhibitor | IM | −0.12131 | −0.03476 | −0.05768 | 0.007586 | −0.09134 |
| K05674516 | sofosbuvir | RNA polymerase inhibitor | IM | −0.06249 | −0.00524 | −0.06006 | −0.06371 | 0.021008 |
| K05804044 | AZ-628 | RAF inhibitor | M | 0.190801 | 0.335575 | 0.113257 | −0.23266 | 0.324717 |
| K05900209 | dexlansoprazole | ATPase inhibitor | M | 0.13562 | 0.067482 | −0.03546 | −0.02941 | 0.057768 |
| K06328344 | bremelanotide | melanocortin receptor agonist | IM | −0.07952 | −0.04735 | −0.04913 | 0.07264 | −0.06366 |
| K06388517 | lofemizole | cyclooxygenase inhibitor | MSL | 0.065259 | 0.067703 | −0.15975 | −0.10659 | 0.036188 |
| K06519765 | vinblastine | microtubule inhibitor; tubulin polymerization inhibitor | MSL | −0.10207 | −0.09395 | −0.06392 | −0.05208 | −0.08085 |
| K06543683 | bisindolylmaleimide-ix | PKC inhibitor | M | 0.188652 | 0.225538 | 0.012389 | −0.23095 | 0.162738 |
| K07106112 | BMS-599626 | EGFR inhibitor; protein tyrosine kinase inhibitor | IM | −0.07697 | −0.19006 | 0.419057 | 0.277631 | −0.02871 |
| K07487750 | WHI-P154 | JAK inhibitor | IM | −0.12449 | −0.227 | 0.215164 | 0.246617 | −0.06543 |
| K07612980 | sparfloxacin | bacterial DNA gyrase inhibitor | IM | −0.14733 | −0.10947 | 0.132562 | 0.10192 | −0.10417 |
| K07625016 | norethisterone-enanthate | contraceptive agent | IM | −0.1312 | −0.05873 | 0.104245 | 0.017742 | 0.009756 |
| K07691486 | roscovitine | CDK inhibitor | IM | −0.04702 | −0.21186 | 0.147224 | 0.093376 | −0.16505 |
| K08542803 | gambogic-acid | caspase activator | M | −0.03027 | 0.068122 | −0.05664 | −0.16814 | −0.00196 |
| K08799216 | pelitinib | EGFR inhibitor | IM | −0.13352 | −0.11387 | 0.308393 | 0.196564 | −0.03925 |
| K08893438 | CCG-50014 | G protein signaling inhibitor | M | 0.040398 | 0.039636 | −0.20627 | −0.11977 | −0.03813 |
| K08953028 | U-0521 | catechol O methyltransferase inhibitor | IM | −0.11147 | −0.0012 | 0.062564 | 0.027492 | −0.01537 |
| K09090523 | firocoxib | cyclooxygenase inhibitor | IM | −0.05889 | −0.06912 | 0.049743 | 0.0558 | −0.0068 |
| K09295674 | carzenide | | IM | −0.06454 | −0.00369 | 0.024693 | 0.030621 | 0.002858 |
| K09397065 | SR-57227A | serotonin receptor agonist | IM | −0.10476 | −0.02879 | −0.00449 | −0.08168 | 0.016349 |
| K09416995 | lovastatin | HMGCR inhibitor | M | 0.051166 | 0.131404 | −0.1344 | −0.27624 | 0.05706 |
| K09436144 | YM022 | CCK receptor antagonist | M | 0.038839 | −0.00497 | −0.08788 | −0.05702 | −0.00858 |
| K09602097 | forskolin | adenylyl cyclase activator | IM | −0.06089 | −0.0681 | −0.02751 | 0.028847 | −0.05334 |
| K09951645 | dabrafenib | RAF inhibitor | M | 0.043133 | 0.163112 | −0.02177 | −0.23346 | 0.14386 |
| K10065684 | dantron | laxative | MSL | −0.0095 | −0.03924 | −0.02064 | 0.094016 | −0.05325 |
| K10670311 | sulfasalazine | cyclooxygenase inhibitor | IM | −0.10126 | −0.11737 | −0.04249 | 0.087243 | −0.03952 |
| K10706131 | rivastigmine | acetylcholinesterase inhibitor | IM | −0.05647 | −0.22825 | 0.041894 | 0.155955 | −0.24721 |
| K11071038 | ICI-162846 | histamine receptor antagonist | IM | −0.09255 | −0.15372 | 0.051701 | 0.164712 | −0.05711 |
| K11094367 | acetanilide | hydrogen peroxide decomposition inhibitor | IM | −0.10712 | −0.16111 | 0.069008 | 0.076528 | −0.10671 |
| K11443721 | NE-100 | sigma receptor antagonist | MSL | 0.093195 | 0.023282 | −0.07257 | −0.05032 | 0.025876 |
| K11717138 | benzbromarone | chloride channel blocker | IM | −0.04729 | −0.08532 | 0.031444 | 0.011952 | −0.0086 |
| K11900042 | methylthiouracil | | IM | −0.05914 | −0.19355 | 0.129781 | 0.131068 | −0.1781 |
| K12040459 | AT7867 | AKT inhibitor | MSL | 0.048117 | 0.046025 | −0.11418 | −0.16648 | 0.003574 |
| K12261274 | zaltidine | histamine receptor antagonist | IM | −0.15234 | −0.04172 | −0.03658 | 0.025681 | −0.09292 |
| K12829205 | mechlorethamine | DNA inhibitor | IM | −0.15666 | −0.07571 | 0.085438 | 0.069184 | −0.05962 |
| K13044802 | ciclopirox | membrane integrity inhibitor | IM | −0.15742 | −0.0735 | 0.084523 | 0.04419 | −0.06749 |
| K13060017 | UNC0631 | histone lysine methyltransferase inhibitor | M | 0.105455 | 0.216782 | −0.09072 | −0.32562 | 0.163017 |
| K13169950 | NSC-3852 | HDAC inhibitor | M | 0.093556 | 0.064254 | −0.09058 | −0.16656 | −0.00012 |
| K13183738 | pentamidine | anti-pneumocystis agent | M | 0.215745 | 0.206933 | −0.0345 | −0.22166 | 0.139961 |
| K13387373 | thonzonium | ATPase inhibitor | M | 0.156672 | 0.143061 | 0.105866 | −0.13758 | 0.082616 |
| K13394247 | radafaxine | dopamine norepinephrine reuptake inhibitor | M | 0.059801 | −0.03966 | −0.18176 | −0.12571 | −0.04236 |
| K13486660 | sulfacarbamide | | IM | −0.09211 | −0.02159 | −0.06285 | −0.02112 | 0.061664 |
| K13533483 | cyclosporine | calcineurin inhibitor | IM | −0.11227 | −0.12262 | 0.261027 | 0.22077 | −0.05214 |
| K13646352 | midostaurin | FLT3 inhibitor; KIT inhibitor; PKC inhibitor | MSL | −0.13545 | −0.04213 | −0.10689 | −0.0718 | −0.0893 |
| K13664493 | naphthoquine-phosphate | antimalarial agent | MSL | 0.032118 | −0.01598 | −0.02275 | 0.034434 | 0.045174 |
| K14276241 | daucosterol | apoptosis stimulant | MSL | −0.04126 | 0.01292 | −0.05449 | −0.01266 | 0.044865 |
| K14385366 | FG-4592 | hypoxia inducible factor prolyl hydroxylase inhibitor | IM | −0.07927 | −0.0646 | 0.048589 | 0.051068 | 0.023371 |
| K14571191 | zometapine | | MSL | 0.091461 | −0.13468 | −0.04215 | 0.046023 | −0.08271 |
| K14796088 | berberine | LDL receptor activator | M | 0.109367 | 0.210348 | 0.061263 | −0.15578 | 0.215589 |
| K15025317 | BAY-11-7082 | NFkB pathway inhibitor | M | −0.00647 | 0.077933 | 0.031696 | −0.14739 | 0.040136 |
| K15164005 | apoptosis-activator-II | carboxylesterase inhibitor | MSL | 0.027079 | 0.103711 | −0.18862 | −0.14548 | 0.07557 |
| K15179879 | carfilzomib | proteasome inhibitor | IM | −0.13982 | −0.0442 | 0.121707 | 0.012663 | 0.008413 |
| K15318383 | ammonium-glycyrrhizinate | thrombin inhibitor | IM | −0.06692 | −0.02605 | 0.097102 | 0.050095 | −0.07896 |
| K15327298 | fmoc-l-leucine | PPAR receptor agonist | M | 0.089354 | −0.05134 | 0.022971 | −0.01114 | −0.03928 |
| K15563106 | phloretin | sodium/glucose cotransporter inhibitor | MSL | 0.024439 | 0.021695 | −0.00079 | −0.06264 | −0.04132 |
| K16180792 | bis(maltolato)oxo- | tyrosine phosphatase inhibitor | M | 0.148517 | 0.047451 | −0.04621 | −0.17431 | 0.090307 |
| | vanadium(IV) | | | | | | | |
| K16730910 | regorafenib | FGFR inhibitor; KIT inhibitor; PDGFR tyrosine kinase | M | 0.096675 | 0.167977 | 0.108553 | −0.16788 | 0.127004 |
| | | receptor inhibitor; RAF inhibitor; RET tyrosine kinase | | | | | | |
| | | inhibitor; VEGFR inhibitor | | | | | | |
| K17075857 | chloroxine | opioid receptor antagonist | MSL | 0.006831 | −0.03689 | −0.01788 | 0.016492 | 0.035386 |
| K17203476 | LY2874455 | FGFR antagonist | IM | −0.14489 | −0.01933 | −0.10214 | −0.0758 | −0.0838 |
| K17223896 | chloroxylenol | ATP synthase inhibitor | IM | −0.10003 | −0.04482 | 0.08067 | 0.1329 | −0.04417 |
| K17294426 | clebopride | dopamine receptor antagonist | IM | −0.08216 | −0.01282 | −0.03223 | 0.070657 | 0.012136 |
| K17497770 | butein | EGFR inhibitor; src inhibitor | MSL | −0.01064 | 0.107801 | −0.08886 | −0.15379 | 0.054997 |
| K17702546 | PD-168393 | EGFR inhibitor | IM | −0.14035 | −0.09474 | 0.313213 | 0.204165 | 0.051791 |
| K17849083 | tranilast | angiogenesis inhibitor | IM | −0.16643 | −0.04394 | −0.01337 | −0.01518 | −0.05076 |
| K18424115 | TG-100572 | src inhibitor; VEGFR inhibitor | MSL | 0.090482 | 0.013923 | −0.04088 | −0.05751 | 0.032211 |
| K18779551 | bifemelane | acetylcholine release enhancer | IM | −0.08356 | −0.05315 | −0.04947 | 0.052284 | −0.05356 |
| K18849474 | dalcetrapib | cholesteryl ester transfer protein inhibitor | IM | −0.05336 | −0.04114 | −0.09318 | 0.036301 | 0.053092 |
| K18898553 | salvianolic-acid-B | metalloproteinase inhibitor; EGFR inhibitor | MSL | −0.10921 | 0.001211 | −0.06128 | −0.08646 | 0.002256 |
| K19061412 | mubritinib | protein tyrosine kinase inhibitor | M | 0.095448 | 0.114313 | 0.061376 | −0.09164 | 0.109507 |
| K19111024 | clofibric-acid | PPAR receptor agonist | MSL | 0.111623 | 0.023677 | −0.12595 | −0.05105 | 0.054907 |
| K19180152 | meprobamate | | IM | −0.15038 | −0.04138 | −0.11396 | −0.02222 | 0.004344 |
| K19333160 | RKI-1447 | rho associated kinase inhibitor | MSL | 0.046328 | 0.093793 | −0.20818 | −0.1921 | 0.081223 |
| K19540840 | saracatinib | src inhibitor | IM | −0.18309 | −0.17375 | 0.216987 | 0.210625 | −0.10679 |
| K19605405 | ZM-241385 | adenosine receptor antagonist | IM | −0.06844 | −0.15131 | 0.027386 | 0.159487 | −0.06032 |
| K19687926 | lapatinib | EGFR inhibitor | IM | −0.08369 | −0.12972 | 0.393376 | 0.251921 | −0.0302 |
| K20093108 | BMS-587101 | integrin antagonist | MSL | 0.019858 | −0.05699 | −0.03676 | 0.136695 | −0.08591 |
| K20285085 | R406 | SYK inhibitor | MSL | −0.1076 | −0.00181 | −0.05554 | −0.02564 | −0.04153 |
| K20372197 | droxidopa | norepinephrine precursor | MSL | −0.01104 | −0.03244 | −0.04537 | −0.02397 | 0.014847 |
| K20468903 | BNC105 | tubulin polymerization inhibitor | MSL | −0.11193 | −0.07446 | −0.09188 | −0.08585 | −0.11348 |
| K20722021 | CEP-32496 | RAF inhibitor | M | 0.165552 | 0.198546 | 0.167139 | −0.15151 | 0.146779 |
| K20920669 | cromoglicic-acid | immunosuppressant | IM | −0.09371 | −0.06183 | −0.05807 | 0.04114 | −0.0649 |
| K21071223 | VP-20629 | beta amyloid antagonist | IM | −0.05479 | −0.10002 | −0.08624 | 0.061572 | −0.10992 |
| K21077415 | AMG-319 | PI3K inhibitor | MSL | −0.0218 | −0.00518 | −0.07748 | −0.12017 | −0.04463 |
| K21152241 | enoximone | phosphodiesterase inhibitor | MSL | 0.070904 | 0.018644 | −0.02835 | −0.04057 | 0.032338 |
| K21191422 | selexipag | platelet aggregation inhibitor; IP1 prostacyclin receptor | MSL | −0.10716 | −0.02894 | −0.08401 | 0.003673 | 0.042186 |
| | | agonist | | | | | | |
| K21237892 | semapimod | cytokine production inhibitor; p38 MAPK inhibitor | MSL | 0.012643 | 0.126689 | −0.0812 | −0.09225 | 0.207005 |
| K21372554 | cefetamet-pivoxil | bacterial cell wall synthesis inhibitor | IM | −0.12174 | −0.01246 | −0.041 | 0.001642 | −0.06679 |
| K21450440 | benzthiazide | carbonic anhydrase inhibitor | M | 0.065069 | −0.0228 | −0.07499 | −0.00741 | −0.08286 |
| K21520694 | sulfacetamide | PABA antagonist | IM | −0.02431 | −0.07824 | −0.05729 | −0.02575 | −0.02586 |
| K21612934 | leteprinim | nerve growth factor agonist | MSL | −0.08293 | 0.085753 | −0.09795 | −0.1262 | 0.025073 |
| K21908111 | AZD1208 | Pim kinase inhibitor | M | 0.034721 | 0.063589 | 0.004575 | −0.02916 | 0.028679 |
| K22134346 | simvastatin | HMGCR inhibitor | M | 0.067288 | 0.085057 | −0.1054 | −0.24598 | 0.0435 |
| K22514639 | EGF816 | EGFR inhibitor | IM | −0.07885 | −0.11656 | 0.293672 | 0.240042 | 0.015562 |
| K22861715 | chrysin | breast cancer resistance protein inhibitor | IM | −0.12997 | −0.02384 | 0.062876 | 0.011338 | 0.032259 |
| K23163214 | dizocilpine-(−) | glutamate receptor antagonist | MSL | 0.027285 | −0.0703 | −0.09183 | −0.00928 | −0.06945 |
| K23190681 | AV-412 | protein tyrosine kinase inhibitor | IM | −0.09469 | −0.16678 | 0.337777 | 0.231695 | −0.04654 |
| K23672206 | BCI-540 | glutamate receptor agonist | MSL | −0.02488 | 0.076892 | −0.18145 | −0.08152 | 0.012343 |
| K23677682 | voreloxin | topoisomerase inhibitor | MSL | −0.04486 | 0.029195 | −0.06474 | −0.09678 | 0.014952 |
| K23925186 | oridonin | BCL inhibitor | M | 0.132126 | 0.205549 | −0.13284 | −0.29026 | 0.182341 |
| K24187789 | VU0238429 | acetylcholine receptor allosteric modulator | M | −0.07773 | 0.044152 | −0.15108 | −0.18678 | −0.02333 |
| K24221957 | pralidoxime-chloride | acetylcholinesterase inhibitor | MSL | 0.007532 | −0.08276 | −0.01101 | −0.02894 | −0.13744 |
| K24258499 | vanillin | | IM | −0.11856 | −0.13911 | 0.098569 | 0.083791 | −0.12984 |
| K24443618 | etebenecid | uricosuric blocker | IM | −0.07421 | −0.00277 | −0.03082 | 0.043708 | 0.052056 |
| K24610819 | amibegron | adrenergic receptor agonist | MSL | 0.007642 | 0.087713 | −0.15737 | −0.07103 | 0.013476 |
| K24666289 | copanlisib | PI3K inhibitor | IM | −0.04959 | −0.11897 | 0.384041 | 0.161827 | −0.08326 |
| K24820488 | CH-170 | | MSL | 0.034145 | −0.08902 | −0.09413 | 0.069434 | −0.11906 |
| K25186396 | tangeritin | cell cycle inhibitor | IM | −0.09411 | −0.06559 | −0.06252 | 0.048909 | −0.01838 |
| K25204779 | pritelivir | helicase primase inhibitor | IM | −0.14069 | −0.00386 | 0.014593 | −0.02645 | −0.005 |
| K25243230 | oxytocin | oxytocin receptor agonist | MSL | 0.041671 | −0.06688 | −0.03975 | 0.017894 | −0.07667 |
| K25494650 | chlorproguanil | dihydrofolate reductase inhibitor | MSL | −0.04792 | 0.065011 | −0.12459 | −0.12927 | 0.038087 |
| K25835157 | PRT062070 | JAK inhibitor; syk inhibitor | M | 0.00956 | 0.130468 | 0.02542 | −0.1204 | 0.09515 |
| K25970317 | resibufogenin | Na/K-ATPase inhibitor | MSL | −0.05263 | −0.07748 | −0.2401 | −0.07864 | −0.09203 |
| K26168087 | magnolol | PPAR receptor agonist | IM | −0.12924 | −0.08293 | 0.002903 | 0.091225 | −0.07745 |
| K26222048 | trans-10, cis-12-Conjugated-linoleic-acid | | IM | −0.10091 | −0.11382 | 0.00293 | 0.05243 | −0.07628 |
| K26530649 | carboxypyridine-disulfide | | MSL | −0.05161 | 0.001847 | −0.11927 | −0.07858 | −0.02977 |
| K26603252 | PD-153035 | EGFR inhibitor | IM | −0.12324 | −0.15333 | 0.391061 | 0.290451 | −0.03709 |
| K26818574 | BIX-01294 | histone lysine methyltransferase inhibitor | M | 0.083765 | 0.302478 | −0.07421 | −0.30356 | 0.246345 |
| K26838195 | AST-1306 | EGFR inhibitor | IM | −0.07988 | −0.06985 | 0.298567 | 0.185083 | 0.043791 |
| K27293958 | tiaramide | anti-inflammatory agent | MSL | −0.01164 | 0.067585 | −0.12292 | −0.13766 | 0.018729 |
| K27737647 | H-89 | PKA inhibitor | M | 0.092753 | −0.02458 | −0.08057 | −0.02505 | −0.07686 |
| K27799744 | cyclovirobuxin-d | calcium channel modulator | M | 0.067206 | 0.03318 | 0.046193 | −0.04158 | 0.026665 |
| K27911943 | chromanol-293B-(−)-[3R,4S] | potassium channel blocker | IM | −0.08029 | −0.09685 | 0.102101 | 0.087737 | −0.09957 |
| K27938825 | ASA-404 | angiogenesis inhibitor | MSL | 0.079591 | 0.041909 | −0.07013 | −0.06289 | 0.008515 |
| K28061410 | beta-lapachone | topoisomerase inhibitor | M | 0.121653 | 0.313665 | −0.04815 | −0.31661 | 0.182082 |
| K28210218 | cephalothin | bacterial cell wall synthesis inhibitor | MSL | 0.045571 | 0.028234 | −0.09175 | −0.04328 | −0.01503 |
| K28217197 | ganaxolone | GABA receptor modulator | IM | −0.089 | −0.14842 | 0.124007 | 0.167991 | −0.10835 |
| K28405228 | mecarbinate | | IM | −0.07862 | −0.08712 | −0.08851 | 0.042383 | −0.09259 |
| K28537285 | chidamide | HDAC inhibitor | MSL | −0.03251 | 0.020078 | −0.01799 | −0.05874 | 0.030949 |
| K28687144 | fosfosal | phosphodiesterase inhibitor | MSL | 0.028863 | 0.007647 | −0.05999 | −0.05618 | −0.01003 |
| K28824103 | genipin | choleretic agent | M | 0.094785 | 0.144955 | −0.1193 | −0.21684 | 0.105229 |
| K29133151 | ICI-185,282 | thromboxane receptor antagonist | IM | −0.09302 | −0.05673 | 0.141827 | 0.071292 | −0.02205 |
| K29322660 | edoxaban | coagulation factor inhibitor | IM | −0.15472 | −0.06457 | 0.002997 | 0.057855 | −0.04528 |
| K29812331 | dimemorfan | sigma receptor agonist | MSL | −0.08644 | −0.05498 | −0.0952 | 0.016349 | −0.01506 |
| K29895144 | SKLB1002 | VEGFR inhibitor | MSL | 0.023841 | 0.014703 | −0.05424 | −0.00342 | 0.037882 |
| K29905972 | axitinib | PDGFR tyrosine kinase receptor inhibitor; VEGFR | M | 0.081599 | 0.070755 | −0.02527 | −0.11161 | 0.004812 |
| | | inhibitor | | | | | | |
| K30159788 | RSV604 | RSV replication inhibitor | MSL | −0.0004 | 0.037533 | −0.20377 | −0.06521 | 0.050206 |
| K30519779 | homoquinolinic-acid | glutamate receptor agonist | IM | −0.11207 | −0.08778 | 0.003236 | 0.056629 | −0.0252 |
| K30572193 | CTEP | glutamate receptor antagonist | IM | −0.16455 | −0.00044 | 0.088796 | 0.015499 | 0.000238 |
| K30577245 | docetaxel | tubulin polymerization inhibitor | IM | −0.13437 | −0.12384 | 0.123481 | 0.068662 | −0.10936 |
| K30933884 | UNBS-5162 | CC chemokine receptor antagonist | M | 0.098439 | 0.162311 | −0.03433 | −0.20286 | 0.091475 |
| K30977212 | PIK-293 | PI3K inhibitor | IM | −0.13973 | −0.06338 | 0.00947 | 0.066004 | 0.024673 |
| K31283835 | tofacitinib | JAK inhibitor | IM | −0.09231 | −0.06137 | −0.00174 | 0.012824 | −0.07575 |
| K31309378 | lucitanib | FGFR inhibitor; VEGFR inhibitor | MSL | −0.05947 | 0.073952 | −0.06451 | −0.12232 | 0.00043 |
| K31476763 | AZD9668 | elastase inhibitor | M | 0.069343 | 0.067743 | 0.009005 | −0.11388 | −0.00254 |
| K31495718 | AZD7687 | diacylglycerol O acyltransferase inhibitor | IM | −0.02143 | −0.05809 | 0.08286 | 0.08898 | −0.03167 |
| K31698212 | icotinib | EGFR inhibitor | IM | −0.13826 | −0.26608 | 0.352891 | 0.310877 | −0.10753 |
| K31866293 | TAK-632 | RAF inhibitor | M | 0.084888 | 0.304244 | 0.04709 | −0.27088 | 0.20887 |
| K31920458 | mestranol | estrogen receptor agonist | IM | −0.07999 | −0.00535 | 0.011648 | 0.007728 | −0.05874 |
| K32256160 | solithromycin | protein synthesis inhibitor | MSL | 0.033302 | −0.13232 | −0.07345 | −0.0286 | −0.14777 |
| K32292990 | CGP-53353 | EGFR inhibitor; PKC inhibitor | IM | −0.10733 | −0.03055 | 0.112914 | −0.03902 | 0.019108 |
| K32744045 | disulfiram | aldehyde dehydrogenase inhibitor; DNA methyltransferase | M | 0.159232 | 0.146768 | −0.07301 | −0.08197 | 0.140484 |
| | | inhibitor; TRPV agonist | | | | | | |
| K33425534 | exemestane | aromatase inhibitor | M | 0.030809 | 0.079207 | −0.04525 | −0.10564 | 0.03291 |
| K33882852 | ZK-93423 | benzodiazepine receptor agonist | M | 0.10975 | 0.206781 | −0.01274 | −0.14794 | 0.176686 |
| K34328244 | MF-101 | estrogen receptor agonist | IM | −0.0589 | −0.09967 | 0.051616 | 0.063156 | −0.04255 |
| K34411947 | streptozotocin | DNA alkylating agent | MSL | 0.036014 | −0.07058 | −0.07788 | 0.076155 | −0.06131 |
| K34445261 | SLV-320 | adenosine receptor antagonist | MSL | 0.085278 | 0.073939 | −0.08758 | −0.10739 | 0.082357 |
| K34672903 | HEMADO | | IM | −0.08651 | −0.0662 | 0.184852 | 0.057367 | −0.09381 |
| K34801930 | AZD5069 | CC chemokine receptor antagonist | IM | −0.151 | −0.07882 | 0.028755 | 0.1362 | −0.00725 |
| K34870043 | adoprazine | dopamine receptor antagonist; serotonin receptor agonist | M | 0.041238 | 0.111176 | 0.045812 | −0.08108 | 0.12551 |
| K35367061 | LY223982 | leukotriene receptor antagonist | MSL | 0.000351 | 0.013234 | −0.01914 | 0.005739 | −0.04973 |
| K36198571 | WAY-170523 | metalloproteinase inhibitor | MSL | 0.051599 | −0.0044 | −0.04718 | −0.01887 | 0.02955 |
| K36258877 | AZ-10417808 | caspase inhibitor | IM | −0.18391 | −0.06367 | 0.258775 | 0.033406 | −0.05539 |
| K36270037 | esculin | antioxidant | IM | −0.05207 | −0.07189 | 0.102078 | 0.055975 | −0.0386 |
| K36386086 | dolutegravir | HIV integrase inhibitor | M | 0.116822 | 0.187969 | −0.06818 | −0.19967 | 0.141021 |
| K36574127 | LOXO-101 | tropomyosin receptor kinase inhibitor | IM | −0.07758 | −0.14625 | 0.151335 | 0.094925 | −0.11517 |
| K36889451 | tetramethylthiuram-monosulfide | | MSL | −0.05667 | 0.011406 | −0.05004 | −0.01495 | −0.0223 |
| K36965586 | m-Chlorophenylbiguanide | | IM | −0.09639 | −0.14796 | 0.11287 | 0.086553 | −0.0968 |
| K37561857 | zardaverine | phosphodiesterase inhibitor | M | 0.178243 | 0.129869 | −0.21466 | −0.19572 | 0.169947 |
| K37590257 | GSK256066 | phosphodiesterase inhibitor | IM | −0.17817 | −0.12278 | 0.068568 | 0.115542 | −0.11693 |
| K37708699 | exisulind | phosphodiesterase inhibitor | MSL | 0.007602 | 0.115747 | −0.1137 | −0.10746 | 0.086911 |
| K37798499 | etoposide | topoisomerase inhibitor | M | −0.03294 | −0.00653 | 0.040224 | −0.09649 | −0.05532 |
| K38140108 | gadodiamide | radiopaque medium | IM | −0.11277 | −0.07426 | −0.03759 | 0.048857 | −0.0433 |
| K38168441 | dabigatran | thrombin inhibitor | MSL | −0.0756 | 0.028068 | −0.08152 | −0.08549 | −0.06551 |
| K38380126 | thiamet-g | GLCNAC phosphotransferase inhibitor | MSL | 0.082065 | −0.00247 | −0.01406 | −0.01394 | 0.003014 |
| K38473998 | cilomilast | phosphodiesterase inhibitor | IM | −0.10002 | −0.03855 | −0.16763 | 0.005967 | −0.11845 |
| K39009484 | entrectinib | ALK tyrosine kinase receptor inhibitor; proto-oncogene | IM | −0.2145 | −0.09031 | 0.081441 | 0.076559 | −0.03072 |
| | | tyrosine protein kinase inhibitor | | | | | | |
| K39120595 | bithionol | autotaxin inhibitor | IM | −0.13854 | −0.07219 | 0.141648 | 0.099233 | −0.03398 |
| K39166528 | ID-8 | | IM | −0.06014 | −0.07214 | 0.071957 | 0.118755 | −0.10241 |
| K39371216 | cyacetacide | | MSL | 0.037813 | −0.19511 | −0.02093 | 0.130175 | −0.11905 |
| K39624409 | ADL5859 | opioid receptor agonist | M | 0.087985 | 0.038602 | −0.06954 | −0.10884 | 0.007408 |
| K39974922 | lenvatinib | FGFR inhibitor; KIT inhibitor; PDGFR tyrosine kinase | MSL | −0.03256 | 0.077346 | −0.10886 | −0.16946 | 0.002695 |
| | | receptor inhibitor; VEGFR inhibitor | | | | | | |
| K40109029 | SB-505124 | ALK tyrosine kinase receptor inhibitor | M | 0.256196 | 0.247451 | −0.01627 | −0.19866 | 0.168251 |
| K40654626 | ditiocarb-sodium-trihydrate | immunostimulant | MSL | −0.01519 | −0.07988 | −0.01951 | 0.031028 | −0.10956 |
| K40718343 | AEE788 | EGFR inhibitor; VEGFR inhibitor | IM | −0.1022 | −0.09967 | 0.382868 | 0.199126 | 0.018756 |
| K40905133 | phenacemide | sodium channel blocker | IM | −0.17177 | −0.08373 | −0.06578 | 0.065998 | 0.021264 |
| K40992116 | parachlorophenol | antiinfective drug | IM | −0.17449 | −0.0181 | −0.04431 | −0.00932 | 0.016576 |
| K41024817 | 4-phenolsulfonic-acid | | IM | −0.15688 | −0.06223 | 0.006866 | 0.027169 | −0.03477 |
| K41256143 | dehydroepiandrosterone | protein synthesis stimulant | IM | −0.13521 | −0.07818 | −0.02423 | 0.066763 | −0.06806 |
| K41438959 | perampanel | glutamate receptor antagonist | IM | −0.11441 | −0.03076 | 0.110379 | −0.00313 | −0.02712 |
| K41859756 | NVP-AUY922 | HSP inhibitor | IM | −0.17335 | −0.03423 | 0.019695 | −0.08198 | −0.03736 |
| K42090719 | trichloroacetic-acid | | IM | −0.0717 | −0.04086 | −0.02085 | 0.089242 | 0.060841 |
| K42673188 | verubulin | tubulin polymerization inhibitor | MSL | −0.09012 | −0.03568 | −0.01487 | −0.06849 | −0.05141 |
| K42805893 | osimertinib | EGFR inhibitor | IM | −0.06781 | −0.05978 | 0.377375 | 0.229006 | 0.075302 |
| K42981054 | mibampator | glutamate receptor modulator | M | 0.097536 | 0.001457 | 0.112583 | −0.03964 | 0.042554 |
| K42991124 | creatinol-phosphate | | IM | −0.08518 | −0.00785 | −0.06276 | 0.001052 | 0.027575 |
| K43410529 | MLN2480 | RAF inhibitor | M | 0.0501 | 0.184598 | 0.00079 | −0.19661 | 0.197592 |
| K43890836 | gemcadiol | antilipemic | MSL | −0.0826 | −0.17678 | −0.01587 | 0.033766 | −0.17307 |
| K44164034 | pumosetrag | serotonin receptor agonist | MSL | −0.05227 | −0.09804 | −0.13909 | 0.052217 | −0.0985 |
| K44590731 | TU-2100 | | IM | −0.04996 | −0.09456 | 0.055172 | 0.032532 | −0.0627 |
| K44777625 | FH1 | hepatocyte function enhancer | IM | −0.08253 | −0.10997 | −0.14259 | 0.012367 | −0.06073 |
| K44974079 | ticagrelor | purinergic receptor antagonist | MSL | 0.023118 | −0.03833 | −0.07713 | −0.06078 | 0.012863 |
| K45014108 | ephedrine | adrenergic receptor agonist | IM | −0.10993 | −0.03098 | −0.05617 | 0.037011 | −0.08521 |
| K45033733 | famciclovir | DNA polymerase inhibitor | IM | −0.13195 | −0.06777 | −0.01906 | 0.054406 | −0.08807 |
| K45114938 | mezlocillin | bacterial cell wall synthesis inhibitor | IM | −0.07537 | −0.03356 | −0.09296 | 0.006699 | −0.03457 |
| K45125084 | zolimidine | mucus protecting agent | M | 0.132957 | 0.03578 | −0.09035 | −0.07228 | 0.058752 |
| K45168550 | SB-756050 | G protein-coupled receptor agonist | IM | −0.07957 | −0.03347 | 0.015771 | −0.03952 | −0.06711 |
| K45293975 | 7-hydroxystaurosporine | CDK inhibitor; CHK inhibitor; PKC inhibitor | IM | −0.15153 | −0.06588 | 0.090881 | 0.06924 | −0.07646 |
| K45437867 | 5-methylfurmethiodide | acetylcholine receptor agonist | IM | −0.14129 | −0.09845 | 0.084403 | 0.067827 | −0.01347 |
| K45519571 | RN-1747 | TRPV agonist | IM | −0.18391 | −0.14405 | 0.126911 | 0.142784 | −0.09773 |
| K45724504 | anguidine | protein synthesis inhibitor | IM | −0.16382 | −0.1066 | 0.208315 | 0.097134 | −0.12405 |
| K45906612 | presatovir | RSV fusion inhibitor | MSL | 0.003391 | 0.08224 | −0.04286 | 0.010238 | −0.00362 |
| K45916615 | danofloxacin | bacterial DNA gyrase inhibitor | IM | −0.09057 | −0.01362 | −0.03133 | −0.03681 | −0.01901 |
| K46386702 | ARRY-334543 | EGFR inhibitor | IM | −0.05992 | −0.1679 | 0.362184 | 0.263188 | −0.03109 |
| K46625559 | ozanimod | sphingosine 1 phosphate receptor agonist | MSL | −0.06933 | −0.01467 | −0.1062 | 0.008869 | −0.00325 |
| K47079459 | cetrimonium | | IM | −0.07274 | −0.04223 | 0.062435 | 0.064439 | −0.01557 |
| K47150025 | KI-8751 | KIT inhibitor; PDGFR tyrosine kinase receptor inhibitor; | M | 0.025124 | 0.103896 | 0.006054 | −0.09761 | 0.037321 |
| | | VEGFR inhibitor | | | | | | |
| K47554101 | eperezolid | bacterial 30S ribosomal subunit inhibitor | IM | −0.07798 | −0.0187 | −0.09524 | −0.02493 | −0.04423 |
| K47598052 | PP-1 | src inhibitor | IM | −0.15774 | −0.05172 | 0.117537 | 0.052592 | −0.12268 |
| K47929404 | vercirnon | CC chemokine receptor antagonist | MSL | −0.01789 | 0.03409 | −0.13167 | −0.07744 | −0.04665 |
| K48068743 | teneligliptin | dipeptidyl peptidase inhibitor | IM | −0.10057 | −0.10714 | −0.06979 | 0.048309 | −0.12459 |
| K48443249 | CNX-774 | Bruton's tyrosine kinase (BTK) inhibitor | M | 0.078022 | 0.093581 | 0.024629 | −0.09506 | 0.138921 |
| K48452630 | SRT2104 | SIRT activator | MSL | −0.07665 | −0.05657 | −0.08685 | 0.069648 | −0.05596 |
| K48830578 | OMDM-2 | FAAH inhibitor | MSL | 0.00432 | 0.078566 | −0.10684 | −0.13149 | 0.088637 |
| K49075727 | nintedanib | FGFR inhibitor; PDGFR tyrosine kinase receptor inhibitor; | MSL | −0.04785 | 0.16111 | −0.09244 | −0.17045 | 0.134821 |
| | | VEGFR inhibitor | | | | | | |
| K49294207 | BIBU-1361 | EGFR inhibitor | IM | −0.06618 | −0.1599 | 0.47829 | 0.251671 | −0.00947 |
| K49313711 | ribitol | | IM | −0.06938 | −0.06045 | 0.062396 | 0.082565 | −0.04956 |
| K49328571 | dasatinib | Bcr-Abl kinase inhibitor; ephrin inhibitor; KIT inhibitor; | IM | −0.17261 | −0.14603 | 0.113298 | 0.097507 | −0.06188 |
| | | PDGFR tyrosine kinase receptor inhibitor; src inhibitor; | | | | | | |
| | | tyrosine kinase inhibitor | | | | | | |
| K49371609 | PIK-75 | DNA protein kinase inhibitor; PI3K inhibitor | IM | −0.11777 | −0.15406 | 0.077057 | 0.127026 | −0.16957 |
| K49481516 | galantamine | acetylcholinesterase inhibitor | IM | −0.148 | −0.19516 | 0.065157 | 0.09079 | −0.20144 |
| K49669041 | BX-912 | pyruvate dehydrogenase kinase inhibitor | M | 0.04083 | 0.154246 | −0.03732 | −0.23675 | 0.035229 |
| K50010139 | poziotinib | EGFR inhibitor | IM | −0.16686 | −0.12615 | 0.344706 | 0.216384 | −0.02553 |
| K50050533 | docosanol | lipase clearing factor inhibitor | IM | −0.06288 | −0.0995 | −0.04968 | 0.094732 | −0.06037 |
| K50293352 | MM77 | serotonin receptor antagonist | IM | −0.12411 | −0.07111 | 0.009431 | 0.078062 | −0.06165 |
| K50325075 | UCL-2077 | slow afterhyperpolarization channel blocker | IM | −0.20055 | −0.10508 | 0.11276 | 0.087806 | −0.07511 |
| K50388907 | fenofibrate | PPAR receptor agonist | IM | −0.11683 | −0.14399 | 0.285186 | 0.231846 | −0.03064 |
| K50417881 | eticlopride | dopamine receptor antagonist | IM | −0.07879 | −0.03475 | −0.14196 | −0.02344 | −0.03396 |
| K51541829 | Ro-25-6981 | | IM | −0.07012 | −0.15131 | −0.0147 | 0.047075 | −0.12466 |
| K51671335 | levosulpiride | dopamine receptor antagonist | MSL | 0.107658 | −0.11408 | −0.00136 | 0.12286 | −0.12095 |
| K51747290 | hydroxyurea | ribonucleotide reductase inhibitor | IM | −0.10487 | −0.06108 | −0.03196 | 0.047566 | −0.05508 |
| K51751936 | alfadolone-acetate | benzodiazepine receptor agonist | IM | −0.12963 | −0.07327 | 0.026722 | 0.070515 | −0.04454 |
| K51770398 | JNJ-17203212 | TRPV antagonist | IM | −0.10372 | −0.03139 | 0.141985 | 0.036549 | −0.00666 |
| K51784806 | alacepril | angiotensin converting enzyme inhibitor | M | 0.004384 | 0.13897 | −0.19551 | −0.13404 | 0.154141 |
| K52020312 | metronidazole | DNA inhibitor | IM | −0.15233 | −0.08969 | 0.053225 | 0.030201 | −0.06039 |
| K52256627 | chlorhexidine | membrane integrity inhibitor | M | 0.14564 | 0.166455 | −0.11132 | −0.15937 | 0.086625 |
| K52408781 | clobazam | GABA benzodiazepine site receptor agonist | IM | −0.10318 | −0.08124 | 0.087132 | 0.051505 | −0.07058 |
| K52914072 | LTA | | MSL | 0.001953 | −0.02051 | −0.06874 | −0.04386 | 0.014603 |
| K53097745 | pterostilbene | cyclooxygenase inhibitor; PPAR receptor agonist | MSL | −0.0588 | −0.08513 | −0.05713 | 0.060629 | −0.06647 |
| K53156626 | 7-methoxytacrine | acetylcholinesterase inhibitor | MSL | −0.03204 | −0.03879 | −0.06604 | −0.03683 | 0.06329 |
| K53195974 | sipatrigine | voltage-gated sodium channel blocker | IM | −0.13153 | −0.00213 | 0.076823 | −0.01806 | 0.012277 |
| K53205568 | afalanine | dopamine receptor agonist | IM | −0.12942 | −0.16637 | 0.063889 | 0.157833 | −0.06352 |
| K53236343 | aprindine | voltage-gated sodium channel blocker | M | 0.005508 | 0.076781 | −0.10961 | −0.10894 | 0.067142 |
| K53581288 | baricitinib | JAK inhibitor | IM | −0.15406 | −0.04059 | −0.08102 | 0.059694 | −0.0321 |
| K53809807 | fosphenytoin | sodium channel blocker | MSL | 0.008138 | −0.11284 | −0.13291 | 0.000687 | −0.10855 |
| K53814070 | ripasudil | rho associated kinase inhibitor | MSL | −0.1492 | −0.06374 | −0.14823 | −0.06435 | −0.05247 |
| K53857191 | risperidone | dopamine receptor antagonist; serotonin receptor | MSL | −0.06307 | −0.11173 | −0.14329 | 0.013526 | −0.11209 |
| | | antagonist | | | | | | |
| K53963539 | vortioxetine | serotonin receptor agonist; serotonin receptor antagonist | IM | −0.12603 | −0.01414 | −0.01638 | −0.02013 | 0.010164 |
| K54247840 | halofuginone | collagenase inhibitor | IM | −0.14225 | −0.08061 | 0.060211 | 0.044536 | −0.04258 |
| K54256913 | MK-1775 | WEE1 kinase inhibitor | IM | −0.09981 | −0.18779 | 0.117528 | 0.165509 | −0.15373 |
| K54339150 | proquazone | cyclooxygenase inhibitor | MSL | 0.009492 | 0.020664 | −0.07792 | −0.09809 | 0.020307 |
| K54395039 | PR-619 | DUB inhibitor | M | 0.099296 | 0.149535 | −0.12228 | −0.22033 | 0.056745 |
| K54634444 | artesunate | DNA synthesis inhibitor | M | 0.132469 | 0.157504 | 0.046551 | −0.17875 | 0.077482 |
| K54770957 | etoricoxib | cyclooxygenase inhibitor | IM | −0.1183 | −0.12895 | 0.02269 | 0.123329 | −0.06605 |
| K54955827 | niraparib | PARP inhibitor | M | 0.035768 | −0.02358 | −0.10001 | −0.10909 | −0.03477 |
| K54997624 | alpelisib | PI3K inhibitor | IM | −0.08851 | −0.04958 | 0.216096 | 0.138375 | 0.020615 |
| K55172746 | apricitabine | nucleoside reverse transcriptase inhibitor | MSL | 0.02836 | 0.026506 | −0.08862 | −0.10951 | −0.04005 |
| K55696337 | topotecan | topoisomerase inhibitor | IM | −0.16199 | −0.03228 | 0.07572 | −0.0378 | −0.05843 |
| K55705469 | penbutolol | adrenergic receptor antagonist | M | 0.039307 | 0.077353 | −0.07801 | −0.07482 | 0.017902 |
| K56032964 | AP26113 | ALK tyrosine kinase receptor inhibitor | IM | −0.11487 | −0.10102 | 0.191859 | 0.114139 | −0.04914 |
| K56211775 | RTA-408 | nitric oxide production inhibitor | IM | −0.14048 | −0.06968 | 0.083236 | 0.017447 | −0.04828 |
| K56277358 | MGCD-265 | VEGFR inhibitor | MSL | −0.05784 | 0.100077 | −0.0439 | −0.07228 | 0.080488 |
| K56405753 | MK-2461 | FGFR inhibitor; VEGFR inhibitor | MSL | −0.03035 | 0.05424 | −0.01237 | −0.12881 | −0.00757 |
| K56483981 | chicago-sky-blue-6b | glutamate inhibitor; macrophage migration inhibiting factor | IM | −0.1611 | −0.17007 | 0.18405 | 0.159884 | −0.07521 |
| | | inhibitor | | | | | | |
| K56614220 | clofazimine | GK0582 inhibitor | M | 0.14707 | 0.152968 | 0.085301 | −0.09284 | 0.106905 |
| K56707426 | nitarsone | | MSL | 0.033888 | −0.01836 | −0.02358 | 0.023462 | −0.04129 |
| K56751279 | Y-39983 | rho associated kinase inhibitor | M | −0.00611 | 0.056164 | −0.17608 | −0.10224 | −0.02374 |
| K56912469 | SAR407899 | rho associated kinase inhibitor | MSL | 0.076732 | 0.003595 | −0.22773 | −0.10519 | −0.00558 |
| K56957086 | dacinostat | HDAC inhibitor | IM | −0.1602 | −0.07883 | 0.10812 | 0.061513 | −0.1024 |
| K56981171 | brigatinib | ALK tyrosine kinase receptor inhibitor; EGFR inhibitor | IM | −0.17279 | −0.04068 | 0.119946 | 0.027015 | 0.040238 |
| K57169635 | dacomitinib | EGFR inhibitor | IM | −0.11009 | −0.07724 | 0.376362 | 0.196894 | 0.022339 |
| K57427145 | ripazepam | benzodiazepine receptor agonist | MSL | 0.073026 | −0.09455 | −0.0411 | 0.050636 | −0.07501 |
| K58466253 | palmatine-chloride | dopamine synthesis inhibitor | M | 0.191556 | 0.117239 | −0.12098 | −0.16452 | 0.060979 |
| K58501140 | TAK-875 | insulin secretagogue | MSL | 0.031822 | 0.00946 | −0.00217 | −0.00346 | −0.03291 |
| K58529924 | ONC201 | AKT inhibitor; MAP kinase inhibitor | M | 0.050772 | 0.147903 | −0.10747 | −0.18719 | 0.104631 |
| K59256312 | gabexate | serine protease inhibitor | M | 0.004068 | 0.006432 | −0.1971 | −0.03992 | −0.01776 |
| K59436580 | tedizolid | bacterial 50S ribosomal subunit inhibitor | M | 0.115866 | 0.163451 | −0.01192 | −0.09248 | 0.137835 |
| K59753975 | vindesine | tubulin polymerization inhibitor | MSL | −0.099 | −0.05229 | −0.07282 | −0.10853 | −0.11151 |
| K60038276 | irbesartan | angiotensin receptor antagonist | IM | −0.12978 | −0.05717 | −0.06191 | 0.024934 | −0.11703 |
| K60130390 | UNC0642 | histone lysine methyltransferase inhibitor | IM | −0.0704 | −0.20493 | 0.178865 | 0.145585 | −0.1219 |
| K60230970 | MG-132 | proteasome inhibitor | IM | −0.16705 | −0.02075 | 0.021603 | 0.015206 | 0.047125 |
| K60237333 | niacin | NAD precursor; vitamin B | IM | −0.09449 | −0.02672 | −0.01502 | −0.01548 | 0.025392 |
| K60241851 | isodibut | aldehyde reductase inhibitor | MSL | 0.050746 | −0.04572 | −0.10394 | −0.03436 | −0.0652 |
| K60369935 | ribavirin | antiviral | M | 0.116243 | 0.115464 | −0.0009 | −0.03039 | 0.084068 |
| K60443845 | chlormidazole | fungal lanosterol demethylase inhibitor | M | 0.20157 | 0.174446 | −0.00691 | −0.16596 | 0.161226 |
| K61097567 | SB-218795 | tachykinin antagonist | IM | −0.13767 | −0.01019 | −0.10474 | 0.017786 | −0.06114 |
| K61279411 | oxaloacetate | glutamate release inhibitor | IM | −0.09347 | −0.03375 | −0.12458 | 0.040808 | −0.00273 |
| K61337602 | naratriptan | serotonin receptor agonist | IM | −0.11416 | −0.05093 | 0.027761 | −0.00581 | 0.051605 |
| K61443650 | thiomersal | other antibiotic | M | −0.05708 | 0.141662 | −0.12375 | −0.2551 | 0.028697 |
| K61688984 | RGFP966 | HDAC inhibitor | MSL | 0.069122 | −0.101 | −0.02113 | −0.03962 | −0.02541 |
| K61717546 | fleroxacin | topoisomerase inhibitor | IM | −0.06537 | −0.08463 | 0.033342 | 0.067758 | −0.09069 |
| K62200014 | anagrelide | phosphodiesterase inhibitor | M | 0.152247 | 0.223472 | −0.1054 | −0.19312 | 0.254063 |
| K62213621 | dihydroartemisinin | antimalarial agent | M | 0.185453 | 0.164342 | 0.016492 | −0.17576 | 0.134506 |
| K62374002 | PI3K-IN-2 | PI3K inhibitor | IM | −0.07052 | −0.0386 | 0.201271 | 0.078617 | 0.01769 |
| K62412084 | para-toluenesulfonamide | | IM | −0.08582 | −0.03201 | −0.02125 | 0.030771 | 0.031819 |
| K62427771 | AS-77 | potassium channel blocker | MSL | −0.00922 | −0.01368 | −0.04012 | −0.04584 | 0.049708 |
| K63150726 | JTE-907 | cannabinoid receptor inverse agonist | IM | −0.13101 | −0.05167 | −0.01924 | 0.087247 | −0.04389 |
| K63630713 | etacrynic-acid | sodium/potassium/chloride transporter inhibitor | IM | −0.15224 | −0.00582 | 0.025267 | −0.04481 | 0.02339 |
| K63712959 | temoporfin | radical formation stimulant | M | −0.028 | 0.01902 | −0.13406 | −0.04512 | 0.030397 |
| K63932022 | 2′-MeCCPA | adenosine receptor agonist | MSL | −0.01341 | −0.06271 | −0.04413 | 0.034523 | −0.0753 |
| K64052750 | gefitinib | EGFR inhibitor | IM | −0.06642 | −0.18805 | 0.441103 | 0.312071 | −0.05639 |
| K64120610 | vinflunine | microtubule inhibitor | M | −0.04762 | −0.03782 | 0.039019 | −0.042 | −0.03707 |
| K64755930 | etazolate | phosphodiesterase inhibitor | IM | −0.13298 | −0.13051 | −0.09327 | 0.031045 | −0.20601 |
| K64835161 | ML167 | CLK inhibitor; DYRK inhibitor | IM | −0.07536 | −0.07672 | 0.017805 | 0.058357 | −0.03284 |
| K64888243 | AZ960 | JAK inhibitor | IM | −0.13497 | −0.0625 | 0.055087 | 0.008122 | −0.07766 |
| K65498798 | abametapir | metalloproteinase inhibitor | MSL | 0.033789 | −0.04148 | −0.12308 | −0.00257 | −0.09619 |
| K65856711 | rolipram-S(+) | phosphodiesterase inhibitor | IM | −0.10432 | −0.03939 | −0.06426 | −0.06383 | −0.07222 |
| K65900713 | caroverine | glutamate receptor antagonist; calcium channel blocker | MSL | 0.05539 | −0.0572 | −0.10428 | 0.001324 | −0.01545 |
| K66094457 | CPP | glutamate receptor antagonist | MSL | −0.04563 | −0.04467 | −0.2411 | −0.01154 | −0.04842 |
| K66175015 | afatinib | EGFR inhibitor | IM | −0.10367 | −0.04742 | 0.314961 | 0.144973 | 0.061442 |
| K66296774 | fluvastatin | HMGCR inhibitor | M | −0.06321 | −0.12209 | 0.040377 | −0.05653 | −0.10262 |
| K66437909 | KT-433 | uricosuric agent | MSL | −0.07552 | −0.02445 | −0.06092 | −0.01366 | 0.017374 |
| K66538826 | amuvatinib | FLT3 inhibitor; KIT inhibitor; PDGFR tyrosine kinase | M | 0.135714 | 0.143845 | 0.028057 | −0.07438 | 0.115285 |
| | | receptor inhibitor; RAD51 inhibitor; RET tyrosine kinase | | | | | | |
| | | inhibitor | | | | | | |
| K66715657 | UH-232-(+) | dopamine receptor antagonist | IM | −0.08225 | −0.10064 | 0.116886 | 0.08495 | −0.11458 |
| K66808046 | oxyquinoline | chelating agent | M | 0.117824 | 0.175696 | −0.04854 | −0.16134 | 0.135313 |
| K66845263 | sophocarpine | | MSL | −0.0048 | −0.09778 | −0.09949 | 0.02582 | −0.08854 |
| K67173685 | 4E1RCat | protein synthesis inhibitor | M | 0.15508 | 0.055641 | 0.010513 | −0.08971 | 0.016269 |
| K67217586 | fenthion | cholinesterase inhibitor | M | 0.012324 | 0.012906 | 0.026896 | −0.06781 | 0.055719 |
| K67578145 | GDC-0879 | RAF inhibitor | M | 0.110094 | 0.209026 | 0.112872 | −0.18739 | 0.15809 |
| K67783091 | haloperidol | dopamine receptor antagonist | IM | −0.11242 | −0.11265 | 0.102477 | 0.035997 | −0.1016 |
| K68336408 | tyrphostin-AG-1478 | EGFR inhibitor | IM | −0.0761 | −0.09525 | 0.343383 | 0.225881 | 0.028937 |
| K68408772 | carboxyamidotriazole | calcium channel blocker | M | 0.111446 | 0.135875 | −0.06044 | −0.08683 | 0.145825 |
| K68432770 | ampicillin | bacterial cell wall synthesis inhibitor | MSL | 0.02394 | −0.05408 | −0.09834 | −0.05206 | −0.06314 |
| K68488863 | ENMD-2076 | Aurora kinase inhibitor; FLT3 inhibitor; VEGFR inhibitor | MSL | −0.05611 | 0.02617 | −0.02079 | −0.10569 | −0.05051 |
| K68553471 | todralazine | antihypertensive agent | IM | −0.0824 | −0.10389 | 0.045407 | 0.054547 | −0.08124 |
| K68747584 | PF-03814735 | Aurora kinase inhibitor | MSL | −0.01193 | 0.015965 | −0.04366 | −0.07539 | −0.05974 |
| K68764924 | pantethine | coenzyme A precursor | IM | −0.12373 | −0.02138 | 0.120562 | 0.041295 | −0.01343 |
| K69726342 | atorvastatin | HMGCR inhibitor | M | 0.115334 | 0.105687 | −0.09939 | −0.24151 | 0.076984 |
| K69907333 | flurbiprofen-(S)-(+) | cyclooxygenase inhibitor | IM | −0.04195 | −0.0194 | 0.029424 | 0.018893 | −0.0319 |
| K70301465 | ibrutinib | Bruton's tyrosine kinase (BTK) inhibitor | IM | −0.0828 | −0.09233 | 0.406517 | 0.23678 | 0.022855 |
| K70487031 | flupentixol | dopamine receptor antagonist | IM | −0.10409 | −0.05449 | 0.153586 | 0.108568 | −0.04775 |
| K70792160 | 10-DEBC | AKT inhibitor | IM | −0.11088 | −0.00116 | −0.03074 | −0.0627 | −0.02503 |
| K70914287 | BIBX-1382 | EGFR inhibitor | IM | −0.03277 | −0.19997 | 0.312955 | 0.250931 | −0.07259 |
| K71075609 | dorzolamide | carbonic anhydrase inhibitor | M | 0.071234 | 0.04159 | −0.08131 | −0.09409 | 0.011006 |
| K71499074 | diclofenamide | carbonic anhydrase inhibitor | MSL | −0.07682 | −0.13011 | −0.00564 | 0.060824 | −0.13556 |
| K71534238 | GW-9508 | free fatty acid receptor agonist; G protein-coupled receptor | MSL | −0.03704 | −0.0358 | −0.05526 | 0.060629 | −0.04742 |
| | | agonist | | | | | | |
| K71926323 | marbofloxacin | bacterial DNA gyrase inhibitor | IM | −0.15249 | −0.03736 | −0.00807 | −0.00013 | −0.03718 |
| K72106461 | SYM-2081 | kainate receptor antagonist | IM | −0.0674 | −0.08842 | 0.05746 | 0.016031 | −0.13111 |
| K72280606 | gilteritinib | FLT3 inhibitor | IM | −0.14704 | −0.04581 | 0.074562 | −0.01504 | −0.01768 |
| K72327355 | baicalein | lipoxygenase inhibitor | IM | −0.14417 | −0.06026 | −0.04061 | 0.01721 | −0.04244 |
| K72414522 | AZD5438 | CDK inhibitor | IM | −0.08372 | −0.14978 | 0.127056 | 0.084401 | −0.13006 |
| K72420232 | WZ-4002 | EGFR inhibitor | IM | −0.07257 | −0.1059 | 0.350678 | 0.190731 | 0.017693 |
| K72533376 | etoposide-phosphate | topoisomerase inhibitor | MSL | −0.06661 | 0.124703 | −0.0333 | −0.17294 | 0.097081 |
| K72723676 | benzethonium | sodium channel blocker | M | 0.156786 | 0.279522 | 0.104147 | −0.21902 | 0.160433 |
| K72951360 | valrubicin | DNA inhibitor; topoisomerase inhibitor | M | −0.00301 | 0.032336 | 0.082401 | −0.06157 | 0.010033 |
| K73027814 | oxyfedrine | adrenergic receptor agonist | MSL | 0.007583 | −0.06224 | −0.05029 | 0.063575 | −0.04763 |
| K73132780 | pyronaridine | antimalarial agent | IM | −0.18189 | −0.18746 | 0.170042 | 0.244639 | −0.13683 |
| K73191876 | alizarin | | IM | −0.16152 | −0.20251 | −0.00609 | 0.195884 | −0.23062 |
| K73309154 | OSI-420 | EGFR inhibitor | IM | −0.11853 | −0.01776 | 0.302701 | 0.141574 | 0.118421 |
| K73319509 | PF-04217903 | c-Met inhibitor | IM | −0.11626 | −0.04418 | 0.077708 | 0.074022 | −0.00139 |
| K73383190 | C646 | histone acetyltransferase inhibitor | MSL | 0.102993 | 0.094869 | −0.10324 | −0.09473 | 0.060774 |
| K73391359 | dimethisoquin | local anesthetic | IM | −0.10101 | −0.05619 | −0.12637 | −0.0517 | −0.0274 |
| K73585091 | GS-9620 | toll-like receptor agonist | MSL | 0.002305 | 0.009784 | −0.15048 | −0.09554 | −0.01739 |
| K73753850 | hexaminolevulinate | | IM | −0.09428 | −0.23539 | 0.182628 | 0.133368 | −0.24324 |
| K73838513 | cinacalcet | calcium channel activator | IM | −0.10749 | −0.06837 | 0.018741 | 0.004349 | −0.00144 |
| K73881242 | arbidol | cytochrome P450 inhibitor | IM | −0.19755 | −0.05354 | 0.049005 | 0.080456 | 0.002591 |
| K74671368 | idronoxil | XIAP inhibitor | MSL | −0.05796 | 0.071135 | −0.14246 | −0.22197 | 0.039836 |
| K74913225 | brinzolamide | carbonic anhydrase inhibitor | IM | −0.15267 | −0.08625 | 0.001342 | 0.017103 | −0.00363 |
| K75080769 | trans-4-[8-(3-Fluorophenyl)-1,7-naphthyridin-6-yl]cyclohexanecarboxylic-acid | | IM | −0.1254 | −0.10859 | 0.041206 | 0.089407 | −0.09561 |
| K75615183 | talipexole | adrenergic receptor agonist; dopamine receptor agonist | M | 0.061351 | −0.01427 | 0.058603 | −0.03535 | −0.08717 |
| K75855670 | iodoquinol | antiseptic | MSL | −0.06766 | 0.03942 | −0.10403 | −0.09894 | 0.031627 |
| K75958547 | pitavastatin | HMGCR inhibitor | MSL | −0.04476 | −0.08119 | −0.0845 | −0.1438 | −0.0837 |
| K76239644 | BMS-690514 | EGFR inhibitor; VEGFR inhibitor | IM | −0.16658 | −0.16482 | 0.346557 | 0.248551 | 0.013462 |
| K76315403 | cetrorelix | gonadotropin releasing factor hormone receptor antagonist | MSL | 0.037232 | −0.18278 | −0.07051 | −0.05533 | −0.13444 |
| K76614248 | atizoram | phosphodiesterase inhibitor | MSL | −0.04268 | −0.0276 | −0.09365 | 0.012063 | 0.000749 |
| K77484724 | adefovir | DNA polymerase inhibitor | IM | −0.05371 | −0.05487 | −0.00969 | −0.00707 | −0.06705 |
| K77561571 | topiroxostat | xanthine oxidase inhibitor | MSL | −0.06728 | −0.14463 | −0.01359 | 0.086231 | −0.19563 |
| K77597856 | detomidine | adrenergic receptor agonist | IM | −0.10768 | −0.13783 | 0.074267 | 0.083014 | −0.09938 |
| K77625799 | vandetanib | EGFR inhibitor; RET tyrosine kinase inhibitor; VEGFR | IM | −0.11628 | −0.04602 | 0.296134 | 0.120843 | 0.049274 |
| | | inhibitor | | | | | | |
| K77627880 | A205804 | ICAMI expression inhibitor | IM | −0.12732 | −0.15624 | 0.073913 | 0.165468 | −0.12354 |
| K77641333 | naphazoline | adrenergic receptor agonist | IM | −0.11467 | −0.08753 | 0.035076 | 0.04142 | −0.00742 |
| K77685957 | dequalinium | PKC inhibitor | M | 0.183809 | −0.00828 | 0.087084 | −0.03298 | 0.003922 |
| K77771411 | moxonidine | imidazoline receptor agonist | IM | −0.08951 | −0.21276 | 0.08201 | 0.139686 | −0.26098 |
| K77791657 | 3-deazaneplanocin-A | histone lysine methyltransferase inhibitor | IM | −0.1309 | −0.02722 | 0.090339 | 0.150724 | −0.05245 |
| K78055238 | LY2090314 | glycogen synthase kinase inhibitor | MSL | −0.08353 | −0.01913 | −0.00714 | 0.041704 | 0.003829 |
| K78096648 | oxiperomide | dopamine receptor antagonist | M | 0.16608 | 0.150247 | −0.19984 | −0.19632 | 0.085408 |
| K78126613 | menadione | mitochondrial DNA polymerase inhibitor; phosphatase | M | 0.004833 | 0.157516 | −0.05102 | −0.20537 | 0.116528 |
| | | inhibitor | | | | | | |
| K78299798 | mepiroxol | | IM | −0.10818 | −0.07855 | 0.017892 | 0.0253 | −0.08258 |
| K78567475 | dolastatin-10 | tubulin polymerization inhibitor | MSL | −0.07579 | −0.05477 | −0.00011 | 0.003299 | −0.08086 |
| K78666826 | procaterol | adrenergic receptor agonist | IM | −0.19402 | −0.22022 | 0.080601 | 0.133962 | −0.1159 |
| K79277568 | methoxyamine | DNA repair enzyme inhibitor | IM | −0.05272 | −0.10687 | −0.03117 | 0.066224 | −0.08534 |
| K79710622 | GKT137831 | NADPH oxidase inhibitor | MSL | −0.01941 | −0.01213 | −0.12349 | −0.11556 | 0.027049 |
| K79821389 | rubitecan | topoisomerase inhibitor | IM | −0.11776 | −0.08132 | 0.164589 | 0.071206 | −0.06417 |
| K80043866 | sodium-gualenate | antacid | IM | −0.0463 | −0.12519 | −0.00503 | 0.05288 | −0.07597 |
| K80343549 | TAK-285 | EGFR inhibitor | IM | −0.07158 | −0.15978 | 0.178479 | 0.222079 | −0.06872 |
| K80353807 | khellin | vasodilator | MSL | −0.05154 | −0.06807 | −0.14627 | −0.06911 | −0.10492 |
| K80359953 | talampanel | glutamate receptor antagonist | MSL | −0.00653 | −0.02993 | −0.00771 | −0.03271 | −0.04869 |
| K80419150 | debrisoquin | adrenergic neuron blocker | MSL | 0.049189 | −0.09369 | −0.11534 | −0.05638 | −0.10054 |
| K80480517 | repsox | TGF beta receptor inhibitor | IM | −0.05665 | −0.07924 | 0.08697 | 0.106197 | −0.03254 |
| K81016934 | INC-280 | c-Met inhibitor | IM | −0.13375 | −0.08364 | −0.03988 | 0.12875 | 0.005689 |
| K81136890 | leucylleucine-methyl-ester | | IM | −0.10897 | −0.1343 | 0.017746 | 0.040373 | −0.18094 |
| K81330143 | tideglusib | glycogen synthase kinase inhibitor | M | 0.169265 | −0.00301 | −0.17514 | −0.01784 | −0.03539 |
| K81332461 | maxacalcitol | vitamin D receptor agonist | IM | −0.07504 | −0.07576 | 0.200324 | 0.130705 | −0.00506 |
| K81473089 | tacrine | acetylcholinesterase inhibitor | IM | −0.14634 | −0.01549 | 0.062641 | 0.03587 | 0.070795 |
| K81548480 | AC-264613 | PAR agonist | IM | −0.07682 | −0.07621 | 0.002402 | 0.093802 | −0.03521 |
| K81645907 | captamine | | IM | −0.14238 | −0.0391 | −0.05026 | 0.012321 | −0.0463 |
| K81672972 | dinoprost | prostacyclin analog | IM | −0.06707 | −0.08325 | −0.07463 | 0.036073 | −0.02378 |
| K81728688 | WZ8040 | EGFR inhibitor | IM | −0.13869 | −0.07504 | 0.289497 | 0.180539 | 0.042302 |
| K81801188 | PP-121 | protein tyrosine kinase inhibitor | IM | −0.12182 | −0.08606 | 0.02379 | 0.078213 | −0.08787 |
| K81807412 | N6-methyladenosine | | IM | −0.11803 | −0.10798 | −0.05829 | 0.022165 | −0.08779 |
| K82028950 | CGH2466 | adenosine receptor antagonist | IM | −0.09319 | −0.07415 | 0.043309 | 0.037758 | −0.01803 |
| K82928847 | ACY-1215 | HDAC inhibitor | M | 0.094251 | 0.158679 | 0.050582 | −0.11818 | 0.218163 |
| K82960980 | ceftiofur | bacterial cell wall synthesis inhibitor | IM | −0.07342 | −0.06755 | 0.082893 | 0.04597 | −0.06957 |
| K82967685 | mirodenafil | phosphodiesterase inhibitor | MSL | −0.05009 | −0.05595 | −0.11332 | 0.072489 | −0.05943 |
| K83144676 | olmesartan | angiotensin receptor antagonist | IM | −0.20014 | −0.04767 | −0.02847 | 0.018055 | −0.0502 |
| K83257731 | chloropyramine | histamine receptor antagonist | M | −0.0016 | 0.120983 | −0.05886 | −0.17825 | 0.083052 |
| K83776863 | tryptophan | serotonin receptor partial agonist | M | 0.044023 | −0.06657 | −0.06507 | −0.01791 | −0.09035 |
| K84011460 | nizofenone | ion channel antagonist | IM | −0.14245 | −0.10603 | 0.033316 | 0.049138 | −0.03805 |
| K84810405 | almorexant | orexin receptor antagonist | IM | −0.14273 | −0.02075 | 0.025983 | −0.02213 | −0.02039 |
| K84868168 | erdafitinib | FGFR inhibitor | MSL | −0.0744 | 0.048316 | −0.15447 | −0.18637 | 0.006092 |
| K85046107 | ramosetron | serotonin receptor antagonist | IM | −0.1044 | −0.2143 | 0.123416 | 0.165677 | −0.22649 |
| K85402309 | dovitinib | EGFR inhibitor; FGFR inhibitor; FLT3 inhibitor; PDGFR | M | −0.00848 | 0.022962 | −0.0224 | −0.09759 | −0.0508 |
| | | tyrosine kinase receptor inhibitor; VEGFR inhibitor | | | | | | |
| K86171477 | eltoprazine | serotonin receptor agonist | IM | −0.05317 | −0.00439 | −0.07472 | −0.08297 | −0.0033 |
| K86204871 | terconazole | sterol demethylase inhibitor | IM | −0.11013 | −0.13134 | −0.03559 | 0.082447 | −0.15851 |
| K86307448 | allopurinol | xanthine oxidase inhibitor | IM | −0.1123 | −0.05119 | 0.037473 | 0.067675 | 0.002066 |
| K86856088 | UNC0638 | histone lysine methyltransferase inhibitor | IM | −0.07586 | −0.12643 | 0.169672 | 0.133158 | −0.08839 |
| K86882815 | cabergoline | dopamine receptor agonist | IM | −0.10406 | −0.0254 | −0.00766 | 0.012508 | 0.071812 |
| K87036601 | azodicarbonamide | DNA synthesis inhibitor | IM | −0.08702 | −0.05349 | 0.0555 | 0.03283 | −0.07574 |
| K87316765 | prinaberel | estrogen receptor agonist | MSL | −0.00721 | 0.004934 | −0.11333 | −0.04804 | 0.029147 |
| K87349682 | norepinephrine | adrenergic receptor agonist | IM | −0.1184 | −0.08076 | 0.105905 | −0.01391 | −0.05347 |
| K87737963 | cyt387 | | MSL | −0.03421 | 0.067632 | −0.14158 | −0.11421 | 0.000569 |
| K88061624 | mizoribine | immunosuppressant; inosine monophosphate | M | 0.005053 | −0.05786 | 0.073386 | −0.00258 | −0.02577 |
| | | dehydrogenase inhibitor | | | | | | |
| K88429204 | pyrimethamine | dihydrofolate reductase inhibitor | MSL | 0.085979 | 0.272942 | −0.01069 | −0.1849 | 0.28398 |
| K88506063 | KD025 | rho associated kinase inhibitor | MSL | 0.057163 | −0.01081 | −0.02606 | −0.10459 | −0.00306 |
| K88544581 | CI-976 | ACAT inhibitor | MSL | 0.091976 | 0.060823 | −0.03363 | −0.082 | 0.03532 |
| K88807631 | elacridar | P glycoprotein inhibitor | IM | −0.11012 | −0.09531 | 0.081367 | 0.121247 | −0.08896 |
| K89053832 | VLX600 | ubiquitin C-terminal hydrolase inhibitor | IM | −0.21353 | −0.12445 | 0.113456 | 0.061139 | −0.12764 |
| K89208535 | indacaterol | adrenergic receptor agonist | IM | −0.1532 | −0.09336 | 0.120724 | 0.05954 | −0.08427 |
| K89299012 | DCEBIO | potassium channel activator | M | 0.157728 | 0.120658 | −0.1352 | −0.11985 | 0.120994 |
| K89413285 | remimazolam | benzodiazepine receptor agonist | MSL | −0.02892 | 0.037853 | −0.10586 | −0.0431 | −0.03395 |
| K89634775 | ally lthiourea | nitrification inhibitor | IM | −0.13254 | −0.04074 | −0.08637 | 0.003499 | −0.0553 |
| K89704198 | vincamine | adrenergic receptor antagonist | IM | −0.06329 | −0.17047 | −0.044 | 0.137068 | −0.17673 |
| K90239174 | AMG-487 | CC chemokine receptor antagonist | IM | −0.0821 | −0.08583 | 0.15574 | 0.047664 | −0.01666 |
| K90825648 | RS-127445 | serotonin receptor antagonist | MSL | 0.054312 | 0.053938 | −0.05973 | −0.12961 | 0.0427 |
| K91758890 | 4-HQN | PARP inhibitor | IM | −0.14748 | −0.06704 | 0.106286 | 0.04595 | 0.05447 |
| K91825936 | ZK811752 | CC chemokine receptor antagonist | M | 0.099526 | 0.109527 | −0.00448 | −0.08132 | 0.042807 |
| K91868854 | 2-[1-(4-piperonyl)piper- | serotonin receptor agonist | MSL | 0.00776 | 0.03417 | −0.05087 | −0.06017 | 0.011505 |
| | azinyl]benzothiazole | | | | | | | |
| K92107055 | pimecrolimus | calcineurin inhibitor | MSL | −0.01575 | 0.080851 | −0.01742 | −0.02862 | 0.077442 |
| K92426617 | esaprazole | | IM | −0.05509 | −0.03721 | −0.02482 | −0.04024 | −0.09876 |
| K92908289 | medica-16 | ATP citrase lyase inhibitor | MSL | −0.05017 | −0.06152 | −0.10856 | 0.016127 | 0.004343 |
| K93123848 | RAF265 | RAF inhibitor; VEGFR inhibitor | M | 0.101093 | 0.240203 | −0.10449 | −0.26903 | 0.191571 |
| K93231391 | ethambutol | bacterial cell wall synthesis inhibitor | IM | −0.10826 | −0.12304 | −0.02031 | 0.10026 | −0.12796 |
| K93240442 | dinitolmide | | IM | −0.13801 | −0.07208 | 0.11087 | 0.035794 | −0.07534 |
| K93632104 | sodium-salicylate | prostanoid receptor antagonist | M | 0.033842 | 0.036068 | 0.025363 | −0.02759 | −0.02882 |
| K94072573 | medronic-acid | bone resorption inhibitor | MSL | 0.072827 | −0.13987 | −0.16332 | 0.024658 | −0.18716 |
| K94176593 | TWS-119 | glycogen synthase kinase inhibitor | IM | −0.16057 | −0.02666 | 0.133541 | 0.00573 | −0.03964 |
| K94239562 | paeonol | anti-inflammatory agent | IM | −0.15024 | −0.06775 | −0.03331 | 0.070898 | −0.06165 |
| K94441233 | mevastatin | HMGCR inhibitor | MSL | −0.00119 | −0.07411 | −0.15027 | −0.13048 | −0.09323 |
| K94455792 | ICG-001 | beta-catenin inhibitor | M | 0.178329 | 0.141485 | 0.019291 | −0.22229 | 0.163484 |
| K94832621 | Y-134 | estrogen receptor antagonist | IM | −0.11641 | −0.12575 | −0.04772 | 0.098443 | −0.09333 |
| K95260951 | asenapine | dopamine receptor antagonist; serotonin receptor | MSL | −0.0115 | 0.067082 | −0.17893 | −0.19572 | 0.033668 |
| | | antagonist | | | | | | |
| K95309561 | dienestrol | estrogen receptor agonist | IM | −0.10994 | −0.05691 | −0.0417 | 0.049847 | −0.02711 |
| K95523387 | OLDA | TRPV agonist | MSL | 0.095843 | 0.0783 | −0.06733 | −0.17424 | 0.086135 |
| K95785537 | PP-2 | src inhibitor | IM | −0.1651 | −0.0904 | 0.092261 | 0.046761 | −0.12532 |
| K96042922 | etanidazole | bacterial cell wall synthesis inhibitor | MSL | −0.03599 | −0.10539 | −0.02357 | 0.032236 | −0.03764 |
| K96188950 | caffeic-acid-phenethyl-ester | HIV integrase inhibitor | MSL | 0.099954 | 0.046287 | −0.14119 | −0.04266 | 0.053387 |
| K96194081 | cepharanthine | NFKB pathway inhibitor | IM | −0.09835 | −0.00833 | 0.098167 | 0.073795 | −0.0749 |
| K96259238 | Y-320 | interleukin inhibitor | IM | −0.17254 | −0.1486 | 0.060313 | 0.193536 | −0.10112 |
| K96344439 | tofogliflozin | sodium/glucose cotransporter inhibitor | IM | −0.10738 | −0.13502 | 0.14142 | 0.052584 | −0.0926 |
| K96424892 | chloramphenicol-palmitate | protein synthesis inhibitor | M | 0.06547 | 0.06128 | −0.09487 | −0.11626 | 0.035937 |
| K96874295 | gonadorelin | gonadotropin releasing factor hormone receptor agonist | MSL | −0.05189 | −0.10614 | −0.12242 | −0.01145 | −0.06774 |
| K97025174 | capadenoson | adenosine receptor agonist | IM | −0.1234 | −0.00185 | −0.06004 | 0.032141 | −0.02665 |
| K97056771 | GNF-2 | Bcr-Abl kinase inhibitor | IM | −0.02935 | −0.03304 | −0.05938 | −0.07177 | −0.02831 |
| K97072811 | TG-003 | CLK inhibitor | MSL | −0.04255 | −0.10494 | −0.10317 | 0.033961 | −0.05387 |
| K97233161 | ABT-491 | platelet activating factor receptor antagonist | IM | −0.10653 | −0.08486 | 0.009812 | 0.046206 | −0.12071 |
| K97521363 | clorsulon | glycolysis inhibitor | IM | −0.11992 | −0.02813 | 0.105406 | 0.000191 | −0.01243 |
| K97564742 | mepyramine | histamine receptor antagonist | IM | −0.07718 | −0.15499 | −0.03152 | 0.077879 | −0.11565 |
| K97799481 | oxtriphylline | adenosine receptor antagonist | MSL | 0.020445 | 0.039012 | −0.03878 | −0.08066 | 0.075905 |
| K97939847 | cridanimod | progesterone receptor agonist | MSL | −0.01816 | −0.20219 | −0.01789 | 0.076081 | −0.17 |
| K98251413 | IOX2 | hypoxia inducible factor inhibitor | IM | −0.0409 | −0.04037 | 0.01906 | 0.090664 | −0.06501 |
| K98357249 | IRL-2500 | endothelin receptor antagonist | IM | −0.06473 | −0.01191 | −0.12142 | 0.007861 | 0.02691 |
| K98572433 | AZD8931 | EGFR inhibitor | IM | −0.08735 | −0.09307 | 0.403442 | 0.220841 | 0.04159 |
| K98769987 | flumazenil | benzodiazepine receptor antagonist | MSL | −0.01893 | 0.075314 | −0.13407 | −0.11148 | −0.00489 |
| K99174507 | cardiogenol-c | cardiomyogenesis inducer | MSL | 0.01043 | −0.05174 | −0.00925 | −0.01984 | −0.07629 |
| K99308954 | sodium-monofluorophosphate | | MSL | −0.02506 | 0.126866 | −0.08529 | −0.13473 | 0.045039 |
| K99447003 | enalaprilat | angiotensin converting enzyme inhibitor | MSL | 0.060401 | 0.034044 | −0.1038 | −0.06381 | −0.03184 |
| K99604664 | tanaproget | progesterone receptor agonist | M | 0.134784 | 0.169943 | −0.04088 | −0.15802 | 0.149326 |
| K99621550 | tubocurarine | acetylcholine receptor antagonist | MSL | −0.01099 | −0.01544 | −0.01951 | 0.057187 | −0.07528 |
| K99946902 | hexylresorcinol | local anesthetic | IM | −0.13985 | −0.04512 | 0.005622 | −0.02627 | −0.03629 |
| K99964838 | bosutinib | Abl kinase inhibitor; Bcr-Abl kinase inhibitor; src inhibitor | IM | −0.11177 | −0.13282 | 0.182795 | 0.161122 | 0.000707 |
| M63173034 | clonixin-lysinate | cyclooxygenase inhibitor | MSL | −0.00484 | −0.06378 | −0.03679 | 0.004709 | −0.01494 |

Among other things, our analyses provided valuable insights including, for example, that compounds annotated with a mechanism of action as EGFR inhibitors were significantly associated with the IM class, compounds with a mechanism of action as RAF inhibitors were significantly associated the M class, and compounds with a mechanism of action as rho associated kinase inhibitors were associated with the MSL class (Table 23B)

TABLE 23B
Mechanism of action and associated subtype for selected
compounds with significant subtype association.

| Mechanisms of Action | IM | MSL | M |
|---|---|---|---|
| adrenergic receptor agonist | 9 | 2 | 0 |
| cyclooxygenase inhibitor | 5 | 5 | 1 |
| EGFR inhibitor | 21 | 0 | 0 |
| phosphodiesterase inhibitor | 5 | 5 | 2 |
| RAF inhibitor | 0 | 0 | 7 |
| rho associated kinase inhibitor | 0 | 6 | 1 |

The compound list was further assessed to limit associated genes to those that were present in a list of genes derived to allow for maximum diversity between clusters defined by the 101 gene signature. This list of genes consisted of a union of 937 genes derived from an analysis of lung squamous, melanoma and sarcoma tumor gene expression data sets, 936 genes from an analysis of lung squamous, lung adeno, and breast tumors, 1525 genes from a union of genes sets determined to give maximum diversity, employing the top five sets that use five unique tumor types, and 2072 genes from a union of the top three gene sets that use nine unique tumor types, resulting in 2595 unique genes. When limited to the genes also present in the cell line expression data set, this resulted in a set of 2198 genes. To remove genes with very low expression and very low variability, genes were further limited to those that had a standard deviation greater than 0.3 across the training cell lines, resulting in a final list of 1954 genes.

In addition to gene expression data, gene dependency data was analyzed. These cancer cell line genetic dependencies were estimated using the DEMETER2 model applied to three large-scale RNAi screening datasets: the Broad Institute Project Achilles, Novartis Project DRIVE, and the Marcotte et al. breast cell line dataset (Marcotte, R. et al. Functional genomic landscape of human breast cancer drivers, vulnerabilities, and resistance. Cell 164, 293-309 (2016)), covering a total of 17309 genes and 712 unique cancer cell lines (James M. McFarland, Zandra V. Ho, Guillaume Kugener, Joshua M. Dempster, Phillip G. Montgomery, Jordan G. Bryan, John M. Krill-Burger, Thomas M. Green, Francisca Vazquez, Jesse S. Boehm, Todd R. Golub, William C. Hahn, David E. Root, Aviad Tsherniak. (2018). Improved estimation of cancer dependencies from large-scale RNAi screens using model-based normalization and data integration. Nature Communications 9, Genetic dependency was determined by examining effects of single-gene knockdowns on cell viability, which may, e.g., provide information about genetic pathways that differ between the biology of diverse cell types. Gene dependence data was downloaded from the Broad Institute (https://depmap.org/portal/download/).

This list of genes (based upon gene expression and gene dependency) was analyzed by Pearson correlation with the compound sensitivity data across the training cell lines. Correlation values, p values, q values and local false discovery rates were calculated. This analysis was repeated using the same set of compounds and genes on the test set of cell lines. A list of genes was prepared for genes associated with compounds wherein (i) the q value of the correlation was less than 0.05 in both training and test sets, (ii) the correlation was negative in both training and test sets (indicating that positive gene expression would indicate increased sensitivity of the cells to the compound), and (iii) the compound had a significant negative correlation with one of the subtypes in the training set and the direction of this correlation was also found in the test set of samples, resulting in a list of 1136 gene: compound correlate pairs (Table 24A). When gene dependency was also examined, the criteria were that p values for correlations with compound sensitivity of the gene expression and gene dependency be less than 0.05 for train and test sets, the correlation was negative in both training and test sets (indicating that positive gene expression would indicate increased sensitivity of cells to the compound) and that the compound had a significant negative correlation with one of the TNBC Types in the training set and the direction of this correlation was also found in the test set of samples resulted in a list of 60 gene: compound correlate pairs (Table 24B). Analysis of these two lists of correlate pairs provided 80 unique compounds (Table 25) and 413 unique genes (Table 26).

TABLE 24A
Gene:compound correlate pairs.

| BROAD | | | | | compound | compound | compound | compound | | DTIO | | DTIO |
|---|---|---|---|---|---|---|---|---|---|---|---|---|
| compound | Compound | Mechanism of | Associated | | gene train | gene train | gene train | gene test | TNBCType | Corr | TNBCType | Corr |
| ID | Name | Action (MoA) | Gene | Associated Gene Family | corr | pvalue | qvalue | corr | train | train | test | test |
| A19777893 | menadione- | vitamin K | CHL1 | atp-dependent dna helicase | −0.16405 | 0.002077 | 0.040711 | −0.24587 | M | 0.003549 | M | 0.136223 |
| | bisulfite | | | ddx11-related | | | | | | | | |
| A19777893 | menadione- | vitamin K | COL19A1 | collagen alpha-1 | −0.20712 | 9.49E−05 | 0.005666 | −0.2615 | M | 0.003549 | M | 0.136223 |
| | bisulfite | | | | | | | | | | | |
| A19777893 | menadione- | vitamin K | DSTYK | dual serine/threonine and | −0.19424 | 0.000256 | 0.010993 | −0.28535 | M | 0.003549 | M | 0.136223 |
| | bisulfite | | | tyrosine protein kinase | | | | | | | | |
| A19777893 | menadione- | vitamin K | FCGR2A | low affinity | −0.1722 | 0.001219 | 0.029507 | −0.25535 | M | 0.003549 | M | 0.136223 |
| | bisulfite | | | immunoglobulin gamma fc | | | | | | | | |
| | | | | region receptor ii-a-related | | | | | | | | |
| A19777893 | menadione- | vitamin K | FCRLA | fc receptor-like a | −0.24011 | 5.56E−06 | 0.000784 | −0.26475 | M | 0.003549 | M | 0.136223 |
| | bisulfite | | | | | | | | | | | |
| A19777893 | menadione- | vitamin K | GHR | growth hormone receptor | −0.17005 | 0.001407 | 0.032213 | −0.29744 | M | 0.003549 | M | 0.136223 |
| | bisulfite | | | | | | | | | | | |
| A19777893 | menadione- | vitamin K | LUM | lumican | −0.19584 | 0.000227 | 0.010178 | −0.32589 | M | 0.003549 | M | 0.136223 |
| | bisulfite | | | | | | | | | | | |
| A19777893 | menadione- | vitamin K | LZTS1 | | −0.19066 | 0.000334 | 0.013076 | −0.31334 | M | 0.003549 | M | 0.136223 |
| | bisulfite | | | | | | | | | | | |
| A19777893 | menadione- | vitamin K | NCKAP5L | nck-associated protein 5-like | −0.16478 | 0.001982 | 0.039571 | −0.3161 | M | 0.003549 | M | 0.136223 |
| | bisulfite | | | | | | | | | | | |
| A19777893 | menadione- | vitamin K | P2RX7 | p2x purinoceptor 7 | −0.20541 | 0.000109 | 0.006199 | −0.25845 | M | 0.003549 | M | 0.136223 |
| | bisulfite | | | | | | | | | | | |
| A19777893 | menadione- | vitamin K | PLEKHO1 | pleckstrin homology | −0.16006 | 0.002672 | 0.047229 | −0.25114 | M | 0.003549 | M | 0.136223 |
| | bisulfite | | | domain-containing family o | | | | | | | | |
| | | | | member 1 | | | | | | | | |
| A19777893 | menadione- | vitamin K | PLEKHO2 | pleckstrin homology | −0.21275 | 6.02E−05 | 0.004166 | −0.30027 | M | 0.003549 | M | 0.136223 |
| | bisulfite | | | domain-containing family o | | | | | | | | |
| | | | | member 2 | | | | | | | | |
| A19777893 | menadione- | vitamin K | PLP1 | myelin proteolipid protein | −0.23319 | 1.04E−05 | 0.001237 | −0.24224 | M | 0.003549 | M | 0.136223 |
| | bisulfite | | | | | | | | | | | |
| A19777893 | menadione- | vitamin K | PLPP7 | inactive phospholipid | −0.18307 | 0.000578 | 0.018506 | −0.28435 | M | 0.003549 | M | 0.136223 |
| | bisulfite | | | phosphatase 7 | | | | | | | | |
| A19777893 | menadione- | vitamin K | QKI | protein quaking | −0.21643 | 4.45E−05 | 0.003384 | −0.27363 | M | 0.003549 | M | 0.136223 |
| | bisulfite | | | | | | | | | | | |
| A19777893 | menadione- | vitamin K | SYNM | asparagine--trna ligase; | −0.17425 | 0.001063 | 0.027159 | −0.25909 | M | 0.003549 | M | 0.136223 |
| | bisulfite | | | mitochondrial-related | | | | | | | | |
| A19777893 | menadione- | vitamin K | TAMALIN | protein tamalin | −0.16412 | 0.002068 | 0.040597 | −0.25395 | M | 0.003549 | M | 0.136223 |
| | bisulfite | | | | | | | | | | | |
| A19777893 | menadione- | vitamin K | WWTR1 | ww domain-containing | −0.17938 | 0.000748 | 0.021763 | −0.25083 | M | 0.003549 | M | 0.136223 |
| | bisulfite | | | transcription regulator | | | | | | | | |
| | | | | protein 1 | | | | | | | | |
| A19777893 | menadione- | vitamin K | ZCCHC24 | | −0.17083 | 0.001336 | 0.031201 | −0.31612 | M | 0.003549 | M | 0.136223 |
| | bisulfite | | | | | | | | | | | |
| A27883417 | alexidine | phosphatidyl- | DLX5 | homeobox protein dlx-5 | −0.1652 | 0.001736 | 0.03657 | −0.30277 | M | 0.209638 | M | 0.100148 |
| | | glycerophosphatase | | | | | | | | | | |
| | | inhibitor | | | | | | | | | | |
| A56085258 | LGX818 | RAF inhibitor | A2M | alpha-2-macroglobulin | −0.16839 | 0.001185 | 0.028993 | −0.25529 | M | 0.095349 | M | 0.14417 |
| A56085258 | LGX818 | RAF inhibitor | ADAM23 | disintegrin and | −0.24066 | 3.01E−06 | 0.000507 | −0.28256 | M | 0.095349 | M | 0.14417 |
| | | | | metalloproteinase domain- | | | | | | | | |
| | | | | containing protein 23 | | | | | | | | |
| A56085258 | LGX818 | RAF inhibitor | AMOTL1 | angiomotin-like protein 1 | −0.16334 | 0.001667 | 0.035676 | −0.24815 | M | 0.095349 | M | 0.14417 |
| A56085258 | LGX818 | RAF inhibitor | APOD | apolipoprotein d | −0.2193 | 2.19E−05 | 0.002075 | −0.31606 | M | 0.095349 | M | 0.14417 |
| A56085258 | LGX818 | RAF inhibitor | ASB2 | ankyrin repeat and socs box | −0.29218 | 1.12E−08 | 8.41E−06 | −0.45115 | M | 0.095349 | M | 0.14417 |
| | | | | protein 2 | | | | | | | | |
| A56085258 | LGX818 | RAF inhibitor | BCL2A1 | bcl-2-related protein a1 | −0.38448 | 2.07E−14 | 6.2E−10 | −0.50814 | M | 0.095349 | M | 0.14417 |
| A56085258 | LGX818 | RAF inhibitor | C3orf70 | upf0524 protein c3orf70 | −0.20964 | 5.06E−05 | 0.0037 | −0.32503 | M | 0.095349 | M | 0.14417 |
| A56085258 | LGX818 | RAF inhibitor | CAMK4 | calcium/calmodulin- | −0.16369 | 0.001629 | 0.035163 | −0.24634 | M | 0.095349 | M | 0.14417 |
| | | | | dependent protein kinase | | | | | | | | |
| | | | | type iv | | | | | | | | |
| A56085258 | LGX818 | RAF inhibitor | CD96 | t-cell surface protein tactile | −0.33386 | 4.96E−11 | 1.41E−07 | −0.38369 | M | 0.095349 | M | 0.14417 |
| A56085258 | LGX818 | RAF inhibitor | CDH19 | cadherin-19 | −0.33522 | 4.09E−11 | 1.25E−07 | −0.43896 | M | 0.095349 | M | 0.14417 |
| A56085258 | LGX818 | RAF inhibitor | CHL1 | atp-dependent dna helicase | −0.29742 | 5.95E−09 | 5.24E−06 | −0.31373 | M | 0.095349 | M | 0.14417 |
| | | | | ddx11-related | | | | | | | | |
| A56085258 | LGX818 | RAF inhibitor | CHST11 | carbohydrate | −0.21346 | 3.65E−05 | 0.002957 | −0.30656 | M | 0.095349 | M | 0.14417 |
| | | | | sulfotransferase 11 | | | | | | | | |
| A56085258 | LGX818 | RAF inhibitor | CIITA | mhc class ii transactivator | −0.19451 | 0.000174 | 0.008515 | −0.30937 | M | 0.095349 | M | 0.14417 |
| A56085258 | LGX818 | RAF inhibitor | CMTM5 | cklf-like marvel | −0.17744 | 0.000627 | 0.019495 | −0.36347 | M | 0.095349 | M | 0.14417 |
| | | | | transmembrane domain- | | | | | | | | |
| | | | | containing protein 5 | | | | | | | | |
| A56085258 | LGX818 | RAF inhibitor | COL19A1 | collagen alpha-1 | −0.33232 | 6.15E−11 | 1.67E−07 | −0.4652 | M | 0.095349 | M | 0.14417 |
| A56085258 | LGX818 | RAF inhibitor | COL9A3 | collagen alpha-3 | −0.20705 | 6.29E−05 | 0.004297 | −0.36967 | M | 0.095349 | M | 0.14417 |
| A56085258 | LGX818 | RAF inhibitor | CSPG4 | chondroitin sulfate | −0.20868 | 5.48E−05 | 0.003907 | −0.32376 | M | 0.095349 | M | 0.14417 |
| | | | | proteoglycan 4 | | | | | | | | |
| A56085258 | LGX818 | RAF inhibitor | CTLA4 | cytotoxic t-lymphocyte | −0.26194 | 3.45E−07 | 0.000106 | −0.34344 | M | 0.095349 | M | 0.14417 |
| | | | | protein 4 | | | | | | | | |
| A56085258 | LGX818 | RAF inhibitor | CUBN | cubilin | −0.18418 | 0.000383 | 0.014241 | −0.26741 | M | 0.095349 | M | 0.14417 |
| A56085258 | LGX818 | RAF inhibitor | CYGB | cytoglobin | −0.29983 | 4.42E−09 | 4.2E−06 | −0.28783 | M | 0.095349 | M | 0.14417 |
| A56085258 | LGX818 | RAF inhibitor | DAAM2 | disheveled-associated | −0.29215 | 1.13E−08 | 8.42E−06 | −0.41447 | M | 0.095349 | M | 0.14417 |
| | | | | activator of morphogenesis 2 | | | | | | | | |
| A56085258 | LGX818 | RAF inhibitor | EDNRB | endothelin receptor type b | −0.37653 | 7.69E−14 | 1.39E−09 | −0.42433 | M | 0.095349 | M | 0.14417 |
| A56085258 | LGX818 | RAF inhibitor | FAM180B | protein fam180b | −0.32502 | 1.68E−10 | 3.65E−07 | −0.35665 | M | 0.095349 | M | 0.14417 |
| A56085258 | LGX818 | RAF inhibitor | FCGR2A | low affinity | −0.2842 | 2.89E−08 | 1.68E−05 | −0.37356 | M | 0.095349 | M | 0.14417 |
| | | | | immunoglobulin gamma fc | | | | | | | | |
| | | | | region receptor ii-a-related | | | | | | | | |
| A56085258 | LGX818 | RAF inhibitor | FCGR2B | low affinity | −0.26403 | 2.76E−07 | 9E−05 | −0.369 | M | 0.095349 | M | 0.14417 |
| | | | | immunoglobulin gamma fc | | | | | | | | |
| | | | | region receptor ii-b | | | | | | | | |
| A56085258 | LGX818 | RAF inhibitor | FCMR | fas apoptotic inhibitory | −0.21888 | 2.27E−05 | 0.002132 | −0.33414 | M | 0.095349 | M | 0.14417 |
| | | | | molecule 3 | | | | | | | | |
| A56085258 | LGX818 | RAF inhibitor | FCRLA | fc receptor-like a | −0.39159 | 6.18E−15 | 2.14E−10 | −0.49471 | M | 0.095349 | M | 0.14417 |
| A56085258 | LGX818 | RAF inhibitor | FLT1 | vascular endothelial growth | −0.2932 | 9.93E−09 | 7.7E−06 | −0.26766 | M | 0.095349 | M | 0.14417 |
| | | | | factor receptor 1 | | | | | | | | |
| A56085258 | LGX818 | RAF inhibitor | GAS7 | growth arrest-specific | −0.28115 | 4.11E−08 | 2.19E−05 | −0.46893 | M | 0.095349 | M | 0.14417 |
| | | | | protein 7 | | | | | | | | |
| A56085258 | LGX818 | RAF inhibitor | GHR | growth hormone receptor | −0.1955 | 0.000161 | 0.008071 | −0.23923 | M | 0.095349 | M | 0.14417 |
| A56085258 | LGX818 | RAF inhibitor | GNG2 | guanine nucleotide-binding | −0.25005 | 1.19E−06 | 0.000259 | −0.25046 | M | 0.095349 | M | 0.14417 |
| | | | | protein g | | | | | | | | |
| A56085258 | LGX818 | RAF inhibitor | GPR55 | g-protein coupled receptor 55 | −0.20031 | 0.000109 | 0.006227 | −0.30141 | M | 0.095349 | M | 0.14417 |
| A56085258 | LGX818 | RAF inhibitor | GPSM3 | g-protein-signaling | −0.33901 | 2.39E−11 | 8.4E−08 | −0.2584 | M | 0.095349 | M | 0.14417 |
| | | | | modulator 3 | | | | | | | | |
| A56085258 | LGX818 | RAF inhibitor | GYPC | glycophorin-c | −0.28769 | 1.92E−08 | 1.25E−05 | −0.29639 | M | 0.095349 | M | 0.14417 |
| A56085258 | LGX818 | RAF inhibitor | HPS5 | hermansky-pudlak | −0.26856 | 1.69E−07 | 6.23E−05 | −0.28264 | M | 0.095349 | M | 0.14417 |
| | | | | syndrome 5 protein | | | | | | | | |
| A56085258 | LGX818 | RAF inhibitor | IL12RB2 | interleukin-12 receptor | −0.31955 | 3.51E−10 | 6.2E−07 | −0.30361 | M | 0.095349 | M | 0.14417 |
| | | | | subunit beta-2 | | | | | | | | |
| A56085258 | LGX818 | RAF inhibitor | IL16 | pro-interleukin-16 | −0.3302 | 8.25E−11 | 2.11E−07 | −0.44457 | M | 0.095349 | M | 0.14417 |
| A56085258 | LGX818 | RAF inhibitor | IL24 | | −0.24515 | 1.94E−06 | 0.000369 | −0.27469 | M | 0.095349 | M | 0.14417 |
| A56085258 | LGX818 | RAF inhibitor | IRF4 | interferon regulatory factor 4 | −0.37471 | 1.03E−13 | 1.66E−09 | −0.43362 | M | 0.095349 | M | 0.14417 |
| A56085258 | LGX818 | RAF inhibitor | ITGB3 | integrin beta-3 | −0.20906 | 5.31E−05 | 0.003823 | −0.3046 | M | 0.095349 | M | 0.14417 |
| A56085258 | LGX818 | RAF inhibitor | ITIH5 | inter-alpha-trypsin inhibitor | −0.19754 | 0.000137 | 0.007246 | −0.29554 | M | 0.095349 | M | 0.14417 |
| | | | | heavy chain h5 | | | | | | | | |
| A56085258 | LGX818 | RAF inhibitor | KCNAB1 | voltage-gated potassium | −0.18156 | 0.000465 | 0.016145 | −0.31645 | M | 0.095349 | M | 0.14417 |
| | | | | channel subunit beta-1 | | | | | | | | |
| A56085258 | LGX818 | RAF inhibitor | KCNJ10 | atp-sensitive inward rectifier | −0.20827 | 5.67E−05 | 0.004002 | −0.41309 | M | 0.095349 | M | 0.14417 |
| | | | | potassium channel 10 | | | | | | | | |
| A56085258 | LGX818 | RAF inhibitor | KDR | vascular endothelial growth | −0.21275 | 3.88E−05 | 0.003081 | −0.23905 | M | 0.095349 | M | 0.14417 |
| | | | | factor receptor 2 | | | | | | | | |
| A56085258 | LGX818 | RAF inhibitor | KIRREL1 | kin of irre-like protein 1 | −0.16428 | 0.001566 | 0.034351 | −0.26274 | M | 0.095349 | M | 0.14417 |
| A56085258 | LGX818 | RAF inhibitor | LPXN | leupaxin | −0.2962 | 6.91E−09 | 5.87E−06 | −0.30671 | M | 0.095349 | M | 0.14417 |
| A56085258 | LGX818 | RAF inhibitor | LSAMP | limbic system-associated | −0.24444 | 2.08E−06 | 0.000388 | −0.25145 | M | 0.095349 | M | 0.14417 |
| | | | | membrane protein | | | | | | | | |
| A56085258 | LGX818 | RAF inhibitor | LYL1 | protein lyl-1 | −0.16805 | 0.001213 | 0.029408 | −0.25896 | M | 0.095349 | M | 0.14417 |
| A56085258 | LGX818 | RAF inhibitor | LZTS1 | | −0.25392 | 7.99E−07 | 0.000194 | −0.27449 | M | 0.095349 | M | 0.14417 |
| A56085258 | LGX818 | RAF inhibitor | MCC | colorectal mutant cancer | −0.21255 | 3.95E−05 | 0.003116 | −0.26368 | M | 0.095349 | M | 0.14417 |
| | | | | protein | | | | | | | | |
| A56085258 | LGX818 | RAF inhibitor | MCOLN2 | mucolipin-2 | −0.2334 | 6.04E−06 | 0.000833 | −0.38009 | M | 0.095349 | M | 0.14417 |
| A56085258 | LGX818 | RAF inhibitor | MIA | melanoma-derived growth | −0.30601 | 2.04E−09 | 2.33E−06 | −0.47935 | M | 0.095349 | M | 0.14417 |
| | | | | regulatory protein | | | | | | | | |
| A56085258 | LGX818 | RAF inhibitor | MMP16 | matrix metalloproteinase-16 | −0.20948 | 5.13E−05 | 0.003736 | −0.25534 | M | 0.095349 | M | 0.14417 |
| A56085258 | LGX818 | RAF inhibitor | NFATC2 | nuclear factor of activated t- | −0.26567 | 2.31E−07 | 7.87E−05 | −0.3784 | M | 0.095349 | M | 0.14417 |
| | | | | cells; cytoplasmic 2 | | | | | | | | |
| A56085258 | LGX818 | RAF inhibitor | NGFR | tumor necrosis factor | −0.21368 | 3.58E−05 | 0.00292 | −0.37252 | M | 0.095349 | M | 0.14417 |
| | | | | receptor superfamily | | | | | | | | |
| | | | | member 16 | | | | | | | | |
| A56085258 | LGX818 | RAF inhibitor | NLGN1 | neuroligin-1 | −0.2824 | 3.56E−08 | 1.94E−05 | −0.25938 | M | 0.095349 | M | 0.14417 |
| A56085258 | LGX818 | RAF inhibitor | NRP2 | protein kinase c-binding | −0.21548 | 3.06E−05 | 0.002622 | −0.35039 | M | 0.095349 | M | 0.14417 |
| | | | | protein nell2 | | | | | | | | |
| A56085258 | LGX818 | RAF inhibitor | NRROS | transforming growth factor | −0.29662 | 6.56E−09 | 5.64E−06 | −0.32609 | M | 0.095349 | M | 0.14417 |
| | | | | beta activator lrrc33 | | | | | | | | |
| A56085258 | LGX818 | RAF inhibitor | P2RX7 | p2x purinoceptor 7 | −0.2082 | 5.71E−05 | 0.00402 | −0.26138 | M | 0.095349 | M | 0.14417 |
| A56085258 | LGX818 | RAF inhibitor | PHYHIP | phytanoyl-coa hydroxylase- | −0.19014 | 0.000244 | 0.010656 | −0.29104 | M | 0.095349 | M | 0.14417 |
| | | | | interacting protein | | | | | | | | |
| A56085258 | LGX818 | RAF inhibitor | PIK3CD | phosphatidylinositol 4; 5- | −0.21879 | 2.29E−05 | 0.002143 | −0.27445 | M | 0.095349 | M | 0.14417 |
| | | | | bisphosphate 3-kinase | | | | | | | | |
| | | | | catalytic subunit delta | | | | | | | | |
| | | | | isoform | | | | | | | | |
| A56085258 | LGX818 | RAF inhibitor | PKNOX2 | homeobox protein pknox2 | −0.23598 | 4.73E−06 | 0.000699 | −0.34598 | M | 0.095349 | M | 0.14417 |
| A56085258 | LGX818 | RAF inhibitor | PLEKHO2 | pleckstrin homology | −0.21473 | 3.27E−05 | 0.002741 | −0.24206 | M | 0.095349 | M | 0.14417 |
| | | | | domain-containing family o | | | | | | | | |
| | | | | member 2 | | | | | | | | |
| A56085258 | LGX818 | RAF inhibitor | PLP1 | myelin proteolipid protein | −0.37272 | 1.43E−13 | 2.07E−09 | −0.47469 | M | 0.095349 | M | 0.14417 |
| A56085258 | LGX818 | RAF inhibitor | PLXNB3 | plexin-b3 | −0.18322 | 0.000411 | 0.014908 | −0.27828 | M | 0.095349 | M | 0.14417 |
| A56085258 | LGX818 | RAF inhibitor | PMP2 | myelin p2 protein | −0.33787 | 2.81E−11 | 9.55E−08 | −0.40268 | M | 0.095349 | M | 0.14417 |
| A56085258 | LGX818 | RAF inhibitor | PTCRA | pre t-cell antigen receptor | −0.3693 | 2.47E−13 | 3.07E−09 | −0.45214 | M | 0.095349 | M | 0.14417 |
| | | | | alpha | | | | | | | | |
| A56085258 | LGX818 | RAF inhibitor | PTPRZ1 | receptor-type tyrosine- | −0.27017 | 1.41E−07 | 5.47E−05 | −0.36042 | M | 0.095349 | M | 0.14417 |
| | | | | protein phosphatase zeta | | | | | | | | |
| A56085258 | LGX818 | RAF inhibitor | RENBP | n-acylglucosamine 2- | −0.32001 | 3.3E−10 | 5.88E−07 | −0.32171 | M | 0.095349 | M | 0.14417 |
| | | | | epimerase | | | | | | | | |
| A56085258 | LGX818 | RAF inhibitor | RGS1 | regulator of g-protein | −0.20343 | 8.48E−05 | 0.005251 | −0.31782 | M | 0.095349 | M | 0.14417 |
| | | | | signaling 1 | | | | | | | | |
| A56085258 | LGX818 | RAF inhibitor | RHOJ | rho-related gtp-binding | −0.33576 | 3.79E−11 | 1.2E−07 | −0.33613 | M | 0.095349 | M | 0.14417 |
| | | | | protein rhoj | | | | | | | | |
| A56085258 | LGX818 | RAF inhibitor | SGCD | delta-sarcoglycan | −0.31183 | 9.67E−10 | 1.35E−06 | −0.38792 | M | 0.095349 | M | 0.14417 |
| A56085258 | LGX818 | RAF inhibitor | SHC4 | shc-transforming protein 4 | −0.29815 | 5.44E−09 | 4.9E−06 | −0.3808 | M | 0.095349 | M | 0.14417 |
| A56085258 | LGX818 | RAF inhibitor | SHROOM4 | protein shroom4 | −0.25342 | 8.42E−07 | 0.000202 | −0.28424 | M | 0.095349 | M | 0.14417 |
| A56085258 | LGX818 | RAF inhibitor | SLC35F1 | solute carrier family 35 | −0.2225 | 1.65E−05 | 0.001709 | −0.39285 | M | 0.095349 | M | 0.14417 |
| | | | | member f1 | | | | | | | | |
| A56085258 | LGX818 | RAF inhibitor | SNX10 | sorting nexin-10 | −0.24148 | 2.78E−06 | 0.000477 | −0.24704 | M | 0.095349 | M | 0.14417 |
| A56085258 | LGX818 | RAF inhibitor | SORCS1 | vps10 domain-containing | −0.25559 | 6.73E−07 | 0.000172 | −0.34323 | M | 0.095349 | M | 0.14417 |
| | | | | receptor sorcs1 | | | | | | | | |
| A56085258 | LGX818 | RAF inhibitor | SOX5 | transcription factor sox-5 | −0.21892 | 2.27E−05 | 0.002127 | −0.28721 | M | 0.095349 | M | 0.14417 |
| A56085258 | LGX818 | RAF inhibitor | SRPX | sushi repeat-containing | −0.31846 | 4.06E−10 | 6.86E−07 | −0.33494 | M | 0.095349 | M | 0.14417 |
| | | | | protein srpx | | | | | | | | |
| A56085258 | LGX818 | RAF inhibitor | ST3GAL5 | lactosylceramide alpha-2; 3- | −0.16324 | 0.001679 | 0.035844 | −0.29017 | M | 0.095349 | M | 0.14417 |
| | | | | sialyltransferase | | | | | | | | |
| A56085258 | LGX818 | RAF inhibitor | ST6GALNAC3 | alpha-n- | −0.25362 | 8.24E−07 | 0.000199 | −0.27653 | M | 0.095349 | M | 0.14417 |
| | | | | acetylgalactosaminide | | | | | | | | |
| | | | | alpha-2; 6-sialyltransferase 3 | | | | | | | | |
| A56085258 | LGX818 | RAF inhibitor | ST8SIA1 | alpha-n-acetylneuraminide | −0.29567 | 7.36E−09 | 6.16E−06 | −0.36897 | M | 0.095349 | M | 0.14417 |
| | | | | alpha-2; 8-sialyltransferase | | | | | | | | |
| A56085258 | LGX818 | RAF inhibitor | STK10 | serine/threonine-protein | −0.23813 | 3.85E−06 | 0.000605 | −0.31685 | M | 0.095349 | M | 0.14417 |
| | | | | kinase 10 | | | | | | | | |
| A56085258 | LGX818 | RAF inhibitor | STK32B | serine/threonine-protein | −0.19392 | 0.000182 | 0.008775 | −0.26921 | M | 0.095349 | M | 0.14417 |
| | | | | kinase 32b | | | | | | | | |
| A56085258 | LGX818 | RAF inhibitor | TAMALIN | protein tamalin | −0.23509 | 5.15E−06 | 0.000741 | −0.33024 | M | 0.095349 | M | 0.14417 |
| A56085258 | LGX818 | RAF inhibitor | TIMP3 | metalloproteinase inhibitor 3 | −0.18821 | 0.000283 | 0.011719 | −0.24226 | M | 0.095349 | M | 0.14417 |
| A56085258 | LGX818 | RAF inhibitor | TMEM229B | transmembrane protein 229b | −0.24112 | 2.88E−06 | 0.00049 | −0.32045 | M | 0.095349 | M | 0.14417 |
| A56085258 | LGX818 | RAF inhibitor | TNFRSF14 | tumor necrosis factor | −0.25238 | 9.36E−07 | 0.000218 | −0.28053 | M | 0.095349 | M | 0.14417 |
| | | | | receptor superfamily | | | | | | | | |
| | | | | member 14 | | | | | | | | |
| A56085258 | LGX818 | RAF inhibitor | TRPV2 | transient receptor potential | −0.36439 | 5.36E−13 | 5.3E−09 | −0.39013 | M | 0.095349 | M | 0.14417 |
| | | | | cation channel subfamily v | | | | | | | | |
| | | | | member 2 | | | | | | | | |
| A56085258 | LGX818 | RAF inhibitor | WARS1 | tryptophan--trna ligase; | −0.16727 | 0.001279 | 0.030381 | −0.27796 | M | 0.095349 | M | 0.14417 |
| | | | | cytoplasmic | | | | | | | | |
| A56085258 | LGX818 | RAF inhibitor | WWTR1 | ww domain-containing | −0.19172 | 0.000216 | 0.00985 | −0.30705 | M | 0.095349 | M | 0.14417 |
| | | | | transcription regulator | | | | | | | | |
| | | | | protein 1 | | | | | | | | |
| A75975749 | bafetinib | Bcr-Abl kinase | ANKRD44 | serine/threonine-protein | −0.18164 | 0.000639 | 0.019713 | −0.30286 | M | 0.119134 | M | 0.165805 |
| | | inhibitor; LYN tyrosine | | phosphatase 6 regulatory | | | | | | | | |
| | | kinase inhibitor | | ankyrin repeat subunit b | | | | | | | | |
| A75975749 | bafetinib | Bcr-Abl kinase | APOD | apolipoprotein d | −0.25176 | 1.84E−06 | 0.000356 | −0.29484 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | ASB2 | ankyrin repeat and socs box | −0.18683 | 0.000442 | 0.015634 | −0.30528 | M | 0.119134 | M | 0.165805 |
| | | inhibitor; LYN tyrosine | | protein 2 | | | | | | | | |
| | | kinase inhibitor | | | | | | | | | | |
| A75975749 | bafetinib | Bcr-Abl kinase | BCL2A1 | bcl-2-related protein a1 | −0.32445 | 5.06E−10 | 8.19E−07 | −0.34688 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | C3orf70 | upf0524 protein c3orf70 | −0.19028 | 0.000344 | 0.013311 | −0.33214 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | CD96 | t-cell surface protein tactile | −0.22373 | 2.4E−05 | 0.002208 | −0.24228 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | CDH19 | cadherin-19 | −0.31627 | 1.43E−09 | 1.82E−06 | −0.29508 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | COL19A1 | collagen alpha-1 | −0.34976 | 1.65E−11 | 6.55E−08 | −0.35543 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | COL9A3 | collagen alpha-3 | −0.24685 | 2.95E−06 | 0.000499 | −0.24857 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | CSPG4 | chondroitin sulfate | −0.26527 | 4.77E−07 | 0.000134 | −0.29153 | M | 0.119134 | M | 0.165805 |
| | | inhibitor; LYN tyrosine | | proteoglycan 4 | | | | | | | | |
| | | kinase inhibitor | | | | | | | | | | |
| A75975749 | bafetinib | Bcr-Abl kinase | CTLA4 | cytotoxic t-lymphocyte | −0.22865 | 1.56E−05 | 0.001649 | −0.34779 | M | 0.119134 | M | 0.165805 |
| | | inhibitor; LYN tyrosine | | protein 4 | | | | | | | | |
| | | kinase inhibitor | | | | | | | | | | |
| A75975749 | bafetinib | Bcr-Abl kinase | DAAM2 | disheveled-associated | −0.23336 | 1.03E−05 | 0.001224 | −0.31521 | M | 0.119134 | M | 0.165805 |
| | | inhibitor; LYN tyrosine | | activator of morphogenesis | | | | | | | | |
| | | kinase inhibitor | | 2 | | | | | | | | |
| A75975749 | bafetinib | Bcr-Abl kinase | DSTYK | dual serine/threonine and | −0.17162 | 0.001268 | 0.030229 | −0.24859 | M | 0.119134 | M | 0.165805 |
| | | inhibitor; LYN tyrosine | | tyrosine protein kinase | | | | | | | | |
| | | kinase inhibitor | | | | | | | | | | |
| A75975749 | bafetinib | Bcr-Abl kinase | EDNRB | endothelin receptor type b | −0.30706 | 4.45E−09 | 4.22E−06 | −0.32819 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | FAM180B | protein fam180b | −0.28668 | 4.78E−08 | 2.46E−05 | −0.34598 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | FCGR2A | low affinity | −0.30101 | 9.19E−09 | 7.28E−06 | −0.31337 | M | 0.119134 | M | 0.165805 |
| | | inhibitor; LYN tyrosine | | immunoglobulin gamma fc | | | | | | | | |
| | | kinase inhibitor | | region receptor ii-a-related | | | | | | | | |
| A75975749 | bafetinib | Bcr-Abl kinase | FCGR2B | low affinity | −0.35978 | 3.91E−12 | 2.15E−08 | −0.29889 | M | 0.119134 | M | 0.165805 |
| | | inhibitor; LYN tyrosine | | immunoglobulin gamma fc | | | | | | | | |
| | | kinase inhibitor | | region receptor ii-b | | | | | | | | |
| A75975749 | bafetinib | Bcr-Abl kinase | FCMR | fas apoptotic inhibitory | −0.18407 | 0.000538 | 0.017725 | −0.24024 | M | 0.119134 | M | 0.165805 |
| | | inhibitor; LYN tyrosine | | molecule 3 | | | | | | | | |
| | | kinase inhibitor | | | | | | | | | | |
| A75975749 | bafetinib | Bcr-Abl kinase | FCRLA | fc receptor-like a | −0.32029 | 8.63E−10 | 1.25E−06 | −0.3519 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | FLT1 | vascular endothelial growth | −0.26882 | 3.3E−07 | 0.000103 | −0.2531 | M | 0.119134 | M | 0.165805 |
| | | inhibitor; LYN tyrosine | | factor receptor 1 | | | | | | | | |
| | | kinase inhibitor | | | | | | | | | | |
| A75975749 | bafetinib | Bcr-Abl kinase | GAS7 | growth arrest-specific | −0.31592 | 1.5E−09 | 1.87E−06 | −0.39688 | M | 0.119134 | M | 0.165805 |
| | | inhibitor; LYN tyrosine | | protein 7 | | | | | | | | |
| | | kinase inhibitor | | | | | | | | | | |
| A75975749 | bafetinib | Bcr-Abl kinase | GNG2 | guanine nucleotide-binding | −0.24382 | 3.93E−06 | 0.000613 | −0.25932 | M | 0.119134 | M | 0.165805 |
| | | inhibitor; LYN tyrosine | | protein g | | | | | | | | |
| | | kinase inhibitor | | | | | | | | | | |
| A75975749 | bafetinib | Bcr-Abl kinase | GPR55 | g-protein coupled receptor | −0.31557 | 1.56E−09 | 1.92E−06 | −0.25541 | M | 0.119134 | M | 0.165805 |
| | | inhibitor; LYN tyrosine | | 55 | | | | | | | | |
| | | kinase inhibitor | | | | | | | | | | |
| A75975749 | bafetinib | Bcr-Abl kinase | IL12RB2 | interleukin-12 receptor | −0.19281 | 0.000285 | 0.011792 | −0.28119 | M | 0.119134 | M | 0.165805 |
| | | inhibitor; LYN tyrosine | | subunit beta-2 | | | | | | | | |
| | | kinase inhibitor | | | | | | | | | | |
| A75975749 | bafetinib | Bcr-Abl kinase | IL 16 | pro-interleukin-16 | −0.35047 | 1.5E−11 | 6.07E−08 | −0.39888 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | IRF4 | interferon regulatory factor | −0.30026 | 1E−08 | 7.75E−06 | −0.36836 | M | 0.119134 | M | 0.165805 |
| | | inhibitor; LYN tyrosine | | 4 | | | | | | | | |
| | | kinase inhibitor | | | | | | | | | | |
| A75975749 | bafetinib | Bcr-Abl kinase | ITGA10 | integrin alpha-10 | −0.24995 | 2.19E−06 | 0.000401 | −0.27543 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | LCP2 | lymphocyte cytosolic | −0.22068 | 3.11E−05 | 0.002648 | −0.25768 | M | 0.119134 | M | 0.165805 |
| | | inhibitor; LYN tyrosine | | protein 2 | | | | | | | | |
| | | kinase inhibitor | | | | | | | | | | |
| A75975749 | bafetinib | Bcr-Abl kinase | LPXN | leupaxin | −0.2009 | 0.000155 | 0.007868 | −0.27465 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | LYL1 | protein lyl-1 | −0.20291 | 0.000132 | 0.007083 | −0.23968 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | LZTS1 | | −0.2615 | 7E−07 | 0.000177 | −0.26423 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | MIA | melanoma-derived growth | −0.28781 | 4.21E−08 | 2.23E−05 | −0.31384 | M | 0.119134 | M | 0.165805 |
| | | inhibitor; LYN tyrosine | | regulatory protein | | | | | | | | |
| | | kinase inhibitor | | | | | | | | | | |
| A75975749 | bafetinib | Bcr-Abl kinase | MMP16 | matrix metalloproteinase-16 | −0.22993 | 1.4E−05 | 0.001524 | −0.25299 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | NES | nestin | −0.26536 | 4.72E−07 | 0.000133 | −0.26762 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | NRROS | transforming growth factor | −0.27791 | 1.26E−07 | 5.03E−05 | −0.30984 | M | 0.119134 | M | 0.165805 |
| | | inhibitor; LYN tyrosine | | beta activator lrrc33 | | | | | | | | |
| | | kinase inhibitor | | | | | | | | | | |
| A75975749 | bafetinib | Bcr-Abl kinase | PKNOX2 | homeobox protein pknox2 | −0.30913 | 3.47E−09 | 3.48E−06 | −0.28024 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | PLP1 | myelin proteolipid protein | −0.34609 | 2.77E−11 | 9.44E−08 | −0.40769 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | PLXNC1 | plexin-c1 | −0.25315 | 1.61E−06 | 0.000322 | −0.25776 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | PMP2 | myelin p2 protein | −0.36606 | 1.54E−12 | 1.15E−08 | −0.28479 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | PTCRA | pre t-cell antigen receptor | −0.39041 | 3.44E−14 | 8.59E−10 | −0.31291 | M | 0.119134 | M | 0.165805 |
| | | inhibitor; LYN tyrosine | | alpha | | | | | | | | |
| | | kinase inhibitor | | | | | | | | | | |
| A75975749 | bafetinib | Bcr-Abl kinase | RENBP | n-acylglucosamine 2- | −0.21826 | 3.82E−05 | 0.003048 | −0.24478 | M | 0.119134 | M | 0.165805 |
| | | inhibitor; LYN tyrosine | | epimerase | | | | | | | | |
| | | kinase inhibitor | | | | | | | | | | |
| A75975749 | bafetinib | Bcr-Abl kinase | RGS1 | regulator of g-protein | −0.2271 | 1.79E−05 | 0.001807 | −0.27054 | M | 0.119134 | M | 0.165805 |
| | | inhibitor; LYN tyrosine | | signaling 1 | | | | | | | | |
| | | kinase inhibitor | | | | | | | | | | |
| A75975749 | bafetinib | Bcr-Abl kinase | RHOJ | rho-related gtp-binding | −0.25427 | 1.44E−06 | 0.000297 | −0.32588 | M | 0.119134 | M | 0.165805 |
| | | inhibitor; LYN tyrosine | | protein rhoj | | | | | | | | |
| | | kinase inhibitor | | | | | | | | | | |
| A75975749 | bafetinib | Bcr-Abl kinase | SCML4 | sex comb on midleg-like | −0.31482 | 1.72E−09 | 2.04E−06 | −0.29816 | M | 0.119134 | M | 0.165805 |
| | | inhibitor; LYN tyrosine | | protein 4 | | | | | | | | |
| | | kinase inhibitor | | | | | | | | | | |
| A75975749 | bafetinib | Bcr-Abl kinase | SGCA | alpha-sarcoglycan | −0.19417 | 0.000258 | 0.011027 | −0.25087 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | SGCD | delta-sarcoglycan | −0.31226 | 2.36E−09 | 2.63E−06 | −0.28906 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | SHC4 | shc-transforming protein 4 | −0.32394 | 5.41E−10 | 8.56E−07 | −0.35193 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | SORCS1 | vps10 domain-containing | −0.3851 | 8.09E−14 | 1.4E−09 | −0.25216 | M | 0.119134 | M | 0.165805 |
| | | inhibitor; LYN tyrosine | | receptor sorcs1 | | | | | | | | |
| | | kinase inhibitor | | | | | | | | | | |
| A75975749 | bafetinib | Bcr-Abl kinase | SOX5 | transcription factor sox-5 | −0.22526 | 2.1E−05 | 0.002013 | −0.33593 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | SRPX | sushi repeat-containing | −0.19752 | 0.0002 | 0.009368 | −0.25374 | M | 0.119134 | M | 0.165805 |
| | | inhibitor; LYN tyrosine | | protein srpx | | | | | | | | |
| | | kinase inhibitor | | | | | | | | | | |
| A75975749 | bafetinib | Bcr-Abl kinase | ST3GAL5 | lactosylceramide alpha-2; 3- | −0.18288 | 0.000586 | 0.018659 | −0.26128 | M | 0.119134 | M | 0.165805 |
| | | inhibitor; LYN tyrosine | | sialyltransferase | | | | | | | | |
| | | kinase inhibitor | | | | | | | | | | |
| A75975749 | bafetinib | Bcr-Abl kinase | ST6GALNAC3 | alpha-n- | −0.19973 | 0.000169 | 0.008361 | −0.3294 | M | 0.119134 | M | 0.165805 |
| | | inhibitor; LYN tyrosine | | acetylgalactosaminide | | | | | | | | |
| | | kinase inhibitor | | alpha-2; 6-sialyltransferase 3 | | | | | | | | |
| A75975749 | bafetinib | Bcr-Abl kinase | ST8SIA1 | alpha-n-acetylneuraminide | −0.28571 | 5.33E−08 | 2.65E−05 | −0.25987 | M | 0.119134 | M | 0.165805 |
| | | inhibitor; LYN tyrosine | | alpha-2; 8-sialyltransferase | | | | | | | | |
| | | kinase inhibitor | | | | | | | | | | |
| A75975749 | bafetinib | Bcr-Abl kinase | STK10 | serine/threonine-protein | −0.18937 | 0.000368 | 0.013886 | −0.28438 | M | 0.119134 | M | 0.165805 |
| | | inhibitor; LYN tyrosine | | kinase 10 | | | | | | | | |
| | | kinase inhibitor | | | | | | | | | | |
| A75975749 | bafetinib | Bcr-Abl kinase | TMEM229B | transmembrane protein 229b | −0.19281 | 0.000285 | 0.011792 | −0.2699 | M | 0.119134 | M | 0.165805 |
| | | inhibitor; LYN tyrosine | | | | | | | | | | |
| | | kinase inhibitor | | | | | | | | | | |
| A75975749 | bafetinib | Bcr-Abl kinase | TRPV2 | transient receptor potential | −0.32538 | 4.49E−10 | 7.43E−07 | −0.35086 | M | 0.119134 | M | 0.165805 |
| | | inhibitor; LYN tyrosine | | cation channel subfamily v | | | | | | | | |
| | | kinase inhibitor | | member 2 | | | | | | | | |
| K01507359 | rifampin | RNA polymerase | SCARF2 | scavenger receptor class f | −0.2038 | 0.000141 | 0.0074 | −0.24517 | M | 0.115452 | M | 0.057333 |
| | | inhibitor | | member 2 | | | | | | | | |
| K01507359 | rifampin | RNA polymerase | SMO | smoothened homolog | −0.17829 | 0.000895 | 0.024378 | −0.25815 | M | 0.115452 | M | 0.057333 |
| | | inhibitor | | | | | | | | | | |
| K03765900 | XL-647 | EGFR inhibitor; | ANKRD65 | ankyrin repeat domain- | −0.28971 | 3.4E−08 | 1.87E−05 | −0.3009 | IM | −0.1539 | IM | −0.05086 |
| | | VEGFR inhibitor | | containing protein 65 | | | | | | | | |
| K03765900 | XL-647 | EGFR inhibitor; | COL17A1 | collagen alpha-1 | −0.32929 | 2.69E−10 | 5.06E−07 | −0.32705 | IM | −0.1539 | IM | −0.05086 |
| | | VEGFR inhibitor | | | | | | | | | | |
| K03765900 | XL-647 | EGFR inhibitor; | CXCL16 | c-x-c motif chemokine 16 | −0.20759 | 9.14E−05 | 0.005516 | −0.28528 | IM | −0.1539 | IM | −0.05086 |
| | | VEGFR inhibitor | | | | | | | | | | |
| K03765900 | XL-647 | EGFR inhibitor; | DAPP1 | dual adapter for | −0.32567 | 4.32E−10 | 7.21E−07 | −0.35713 | IM | −0.1539 | IM | −0.05086 |
| | | VEGFR inhibitor | | phosphotyrosine and 3- | | | | | | | | |
| | | | | phosphotyrosine and 3- | | | | | | | | |
| | | | | phosphoinositide | | | | | | | | |
| K03765900 | XL-647 | EGFR inhibitor; | DENNDIC | denn domain-containing | −0.23544 | 8.52E−06 | 0.00107 | −0.29093 | IM | −0.1539 | IM | −0.05086 |
| | | VEGFR inhibitor | | protein 1c | | | | | | | | |
| K03765900 | XL-647 | EGFR inhibitor; | DSG3 | desmoglein-3 | −0.37003 | 8.47E−13 | 7.33E−09 | −0.33926 | IM | −0.1539 | IM | −0.05086 |
| | | VEGFR inhibitor | | | | | | | | | | |
| K03765900 | XL-647 | EGFR inhibitor; | FBLN1 | fibulin-1 | −0.17862 | 0.000789 | 0.022494 | −0.26582 | IM | −0.1539 | IM | −0.05086 |
| | | VEGFR inhibitor | | | | | | | | | | |
| K03765900 | XL-647 | EGFR inhibitor; | FGD3 | fyve; rhogef and ph domain- | −0.18603 | 0.000468 | 0.016223 | −0.31842 | IM | −0.1539 | IM | −0.05086 |
| | | VEGFR inhibitor | | containing protein 3 | | | | | | | | |
| K03765900 | XL-647 | EGFR inhibitor; | ITGB4 | integrin beta-4 | −0.3122 | 2.38E−09 | 2.64E−06 | −0.37313 | IM | −0.1539 | IM | −0.05086 |
| | | VEGFR inhibitor | | | | | | | | | | |
| K03765900 | XL-647 | EGFR inhibitor; | KRT14 | keratin; type i cytoskeletal | −0.33812 | 8.28E−11 | 2.11E−07 | −0.25953 | IM | −0.1539 | IM | −0.05086 |
| | | VEGFR inhibitor | | 14 | | | | | | | | |
| K03765900 | XL-647 | EGFR inhibitor; | KRT16 | keratin; type i cytoskeletal | −0.324 | 5.37E−10 | 8.51E−07 | −0.39867 | IM | −0.1539 | IM | −0.05086 |
| | | VEGFR inhibitor | | 16 | | | | | | | | |
| K03765900 | XL-647 | EGFR inhibitor; | KRT17 | keratin; type i cytoskeletal | −0.33778 | 8.68E−11 | 2.18E−07 | −0.28994 | IM | −0.1539 | IM | −0.05086 |
| | | VEGFR inhibitor | | 17 | | | | | | | | |
| K03765900 | XL-647 | EGFR inhibitor; | KRT5 | keratin; type ii cytoskeletal | −0.3793 | 2.03E−13 | 2.6E−09 | −0.36773 | IM | −0.1539 | IM | −0.05086 |
| | | VEGFR inhibitor | | 5 | | | | | | | | |
| K03765900 | XL-647 | EGFR inhibitor; | MAPK10 | mitogen-activated protein | −0.19522 | 0.000238 | 0.010489 | −0.24025 | IM | −0.1539 | IM | −0.05086 |
| | | VEGFR inhibitor | | kinase 10 | | | | | | | | |
| K03765900 | XL-647 | EGFR inhibitor; | PLAAT4 | phospholipase a and | −0.22419 | 2.3E−05 | 0.002149 | −0.23913 | IM | −0.1539 | IM | −0.05086 |
| | | VEGFR inhibitor | | acyltransferase 4 | | | | | | | | |
| K03765900 | XL-647 | EGFR inhibitor; | PTAFR | platelet-activating factor | −0.32275 | 6.3E−10 | 9.65E−07 | −0.40649 | IM | −0.1539 | IM | −0.05086 |
| | | VEGFR inhibitor | | receptor | | | | | | | | |
| K03765900 | XL-647 | EGFR inhibitor; | PTPN6 | tyrosine-protein phosphatase | −0.30807 | 3.94E−09 | 3.85E−06 | −0.36363 | IM | −0.1539 | IM | −0.05086 |
| | | VEGFR inhibitor | | non-receptor type 6 | | | | | | | | |
| K03765900 | XL-647 | EGFR inhibitor; | S100A8 | protein s100-a8 | −0.24965 | 2.26E−06 | 0.000409 | −0.31627 | IM | −0.1539 | IM | −0.05086 |
| | | VEGFR inhibitor | | | | | | | | | | |
| K03765900 | XL-647 | EGFR inhibitor; | SIRPB2 | signal-regulatory protein | −0.25355 | 1.55E−06 | 0.000314 | −0.29342 | IM | −0.1539 | IM | −0.05086 |
| | | VEGFR inhibitor | | beta-2 | | | | | | | | |
| K03765900 | XL-647 | EGFR inhibitor; | TNF AIP8 | tumor necrosis factor alpha- | −0.31651 | 1.39E−09 | 1.78E−06 | −0.25194 | IM | −0.1539 | IM | −0.05086 |
| | | VEGFR inhibitor | | induced protein 8 | | | | | | | | |
| K03765900 | XL-647 | EGFR inhibitor; | TNFSF10 | tumor necrosis factor ligand | −0.322 | 6.93E−10 | 1.04E−06 | −0.31438 | IM | −0.1539 | IM | −0.05086 |
| | | VEGFR inhibitor | | superfamily member 10 | | | | | | | | |
| K04568635 | octenidine | membrane integrity | ADAM11 | disintegrin and | −0.18264 | 0.000506 | 0.017021 | −0.2787 | M | 0.147947 | M | 0.09423 |
| | | inhibitor | | metalloproteinase domain- | | | | | | | | |
| | | | | containing protein 11 | | | | | | | | |
| K04568635 | octenidine | membrane integrity | CERS1 | ceramide synthase 1 | −0.17362 | 0.000955 | 0.025405 | −0.23784 | M | 0.147947 | M | 0.09423 |
| | | inhibitor | | | | | | | | | | |
| K04568635 | octenidine | membrane integrity | FAM78A | protein fam78a | −0.23004 | 1.07E−05 | 0.00126 | −0.28623 | M | 0.147947 | M | 0.09423 |
| | | inhibitor | | | | | | | | | | |
| K05804044 | AZ-628 | RAF inhibitor | A2M | alpha-2-macroglobulin | −0.21111 | 4.91E−05 | 0.003627 | −0.27125 | M | 0.190801 | M | 0.324717 |
| K05804044 | AZ-628 | RAF inhibitor | AEBP1 | adipocyte enhancer-binding | −0.17089 | 0.001063 | 0.02716 | −0.26693 | M | 0.190801 | M | 0.324717 |
| | | | | protein 1 | | | | | | | | |
| K05804044 | AZ-628 | RAF inhibitor | APOD | apolipoprotein d | −0.22873 | 1.05E−05 | 0.001239 | −0.41082 | M | 0.190801 | M | 0.324717 |
| K05804044 | AZ-628 | RAF inhibitor | ASB2 | ankyrin repeat and socs box | −0.24031 | 3.53E−06 | 0.000571 | −0.36012 | M | 0.190801 | M | 0.324717 |
| | | | | protein 2 | | | | | | | | |
| K05804044 | AZ-628 | RAF inhibitor | BCL2A1 | bcl-2-related protein a1 | −0.3244 | 2.29E−10 | 4.48E−07 | −0.36448 | M | 0.190801 | M | 0.324717 |
| K05804044 | AZ-628 | RAF inhibitor | C3orf70 | upf0524 protein c3orf70 | −0.25107 | 1.22E−06 | 0.000265 | −0.32375 | M | 0.190801 | M | 0.324717 |
| K05804044 | AZ-628 | RAF inhibitor | CDH19 | cadherin-19 | −0.36232 | 9.85E−13 | 8.42E−09 | −0.33307 | M | 0.190801 | M | 0.324717 |
| K05804044 | AZ-628 | RAF inhibitor | CHL1 | atp-dependent dna helicase | −0.23777 | 4.5E−06 | 0.000673 | −0.24278 | M | 0.190801 | M | 0.324717 |
| | | | | ddx11-related | | | | | | | | |
| K05804044 | AZ-628 | RAF inhibitor | CMTM5 | cklf-like marvel | −0.17157 | 0.001014 | 0.026393 | −0.2448 | M | 0.190801 | M | 0.324717 |
| | | | | transmembrane domain- | | | | | | | | |
| | | | | containing protein 5 | | | | | | | | |
| K05804044 | AZ-628 | RAF inhibitor | COL19A1 | collagen alpha-1 | −0.30838 | 1.85E−09 | 2.16E−06 | −0.41797 | M | 0.190801 | M | 0.324717 |
| K05804044 | AZ-628 | RAF inhibitor | COL9A3 | collagen alpha-3 | −0.32496 | 2.13E−10 | 4.22E−07 | −0.39502 | M | 0.190801 | M | 0.324717 |
| K05804044 | AZ-628 | RAF inhibitor | CSPG4 | chondroitin sulfate | −0.27418 | 1.07E−07 | 4.46E−05 | −0.27405 | M | 0.190801 | M | 0.324717 |
| | | | | proteoglycan 4 | | | | | | | | |
| K05804044 | AZ-628 | RAF inhibitor | CTLA4 | cytotoxic t-lymphocyte | −0.24944 | 1.44E−06 | 0.000298 | −0.29473 | M | 0.190801 | M | 0.324717 |
| | | | | protein 4 | | | | | | | | |
| K05804044 | AZ-628 | RAF inhibitor | CYGB | cytoglobin | −0.27288 | 1.23E−07 | 4.96E−05 | −0.34369 | M | 0.190801 | M | 0.324717 |
| K05804044 | AZ-628 | RAF inhibitor | DAAM2 | disheveled-associated | −0.23311 | 6.98E−06 | 0.000926 | −0.30063 | M | 0.190801 | M | 0.324717 |
| | | | | activator of morphogenesis 2 | | | | | | | | |
| K05804044 | AZ-628 | RAF inhibitor | EDNRB | endothelin receptor type b | −0.3548 | 3.08E−12 | 1.82E−08 | −0.33929 | M | 0.190801 | M | 0.324717 |
| K05804044 | AZ-628 | RAF inhibitor | FAM180B | protein fam180b | −0.32371 | 2.52E−10 | 4.84E−07 | −0.39105 | M | 0.190801 | M | 0.324717 |
| K05804044 | AZ-628 | RAF inhibitor | FCER1G | high affinity | −0.21898 | 2.5E−05 | 0.002275 | −0.29011 | M | 0.190801 | M | 0.324717 |
| | | | | immunoglobulin epsilon | | | | | | | | |
| | | | | receptor subunit gamma | | | | | | | | |
| K05804044 | AZ-628 | RAF inhibitor | FCGR2A | low affinity | −0.25237 | 1.07E−06 | 0.000242 | −0.28154 | M | 0.190801 | M | 0.324717 |
| | | | | immunoglobulin gamma fc | | | | | | | | |
| | | | | region receptor ii-a-related | | | | | | | | |
| K05804044 | AZ-628 | RAF inhibitor | FCGR2B | low affinity | −0.26349 | 3.39E−07 | 0.000105 | −0.30444 | M | 0.190801 | M | 0.324717 |
| | | | | immunoglobulin gamma fc | | | | | | | | |
| | | | | region receptor ii-b | | | | | | | | |
| K05804044 | AZ-628 | RAF inhibitor | FCMR | fas apoptotic inhibitory | −0.16428 | 0.001662 | 0.035608 | −0.28415 | M | 0.190801 | M | 0.324717 |
| | | | | molecule 3 | | | | | | | | |
| K05804044 | AZ-628 | RAF inhibitor | FCRLA | fc receptor-like a | −0.35543 | 2.8E−12 | 1.7E−08 | −0.41839 | M | 0.190801 | M | 0.324717 |
| K05804044 | AZ-628 | RAF inhibitor | FLT1 | vascular endothelial growth | −0.30989 | 1.53E−09 | 1.89E−06 | −0.26232 | M | 0.190801 | M | 0.324717 |
| | | | | factor receptor 1 | | | | | | | | |
| K05804044 | AZ-628 | RAF inhibitor | GAS7 | growth arrest-specific | −0.36714 | 4.66E−13 | 4.86E−09 | −0.43906 | M | 0.190801 | M | 0.324717 |
| | | | | protein 7 | | | | | | | | |
| K05804044 | AZ-628 | RAF inhibitor | GHR | growth hormone receptor | −0.22392 | 1.62E−05 | 0.001687 | −0.28625 | M | 0.190801 | M | 0.324717 |
| K05804044 | AZ-628 | RAF inhibitor | GNG2 | guanine nucleotide-binding | −0.25017 | 1.34E−06 | 0.000282 | −0.30488 | M | 0.190801 | M | 0.324717 |
| | | | | protein g | | | | | | | | |
| K05804044 | AZ-628 | RAF inhibitor | GNG7 | guanine nucleotide-binding | −0.20408 | 8.79E−05 | 0.005379 | −0.27393 | M | 0.190801 | M | 0.324717 |
| | | | | protein g | | | | | | | | |
| K05804044 | AZ-628 | RAF inhibitor | GPR55 | g-protein coupled receptor 55 | −0.22022 | 2.24E−05 | 0.002112 | −0.28355 | M | 0.190801 | M | 0.324717 |
| K05804044 | AZ-628 | RAF inhibitor | GSN | gelsolin | −0.24292 | 2.74E−06 | 0.000473 | −0.28737 | M | 0.190801 | M | 0.324717 |
| K05804044 | AZ-628 | RAF inhibitor | IL12RB2 | interleukin-12 receptor | −0.34893 | 7.35E−12 | 3.52E−08 | −0.29022 | M | 0.190801 | M | 0.324717 |
| | | | | subunit beta-2 | | | | | | | | |
| K05804044 | AZ-628 | RAF inhibitor | IL16 | pro-interleukin-16 | −0.33604 | 4.65E−11 | 1.37E−07 | −0.48581 | M | 0.190801 | M | 0.324717 |
| K05804044 | AZ-628 | RAF inhibitor | IRF4 | interferon regulatory factor 4 | −0.34483 | 1.33E−11 | 5.54E−08 | −0.41488 | M | 0.190801 | M | 0.324717 |
| K05804044 | AZ-628 | RAF inhibitor | ITGA10 | integrin alpha-10 | −0.16659 | 0.001424 | 0.032446 | −0.2449 | M | 0.190801 | M | 0.324717 |
| K05804044 | AZ-628 | RAF inhibitor | ITGA9 | integrin alpha-9 | −0.27837 | 6.67E−08 | 3.12E−05 | −0.24282 | M | 0.190801 | M | 0.324717 |
| K05804044 | AZ-628 | RAF inhibitor | KCNAB1 | voltage-gated potassium | −0.21814 | 2.69E−05 | 0.002394 | −0.24308 | M | 0.190801 | M | 0.324717 |
| | | | | channel subunit beta-1 | | | | | | | | |
| K05804044 | AZ-628 | RAF inhibitor | KCNJ10 | atp-sensitive inward rectifier | −0.22282 | 1.78E−05 | 0.001801 | −0.3229 | M | 0.190801 | M | 0.324717 |
| | | | | potassium channel 10 | | | | | | | | |
| K05804044 | AZ-628 | RAF inhibitor | LZTS1 | | −0.22945 | 9.79E−06 | 0.001182 | −0.3171 | M | 0.190801 | M | 0.324717 |
| K05804044 | AZ-628 | RAF inhibitor | MCC | colorectal mutant cancer | −0.1989 | 0.000134 | 0.00713 | −0.26763 | M | 0.190801 | M | 0.324717 |
| | | | | protein | | | | | | | | |
| K05804044 | AZ-628 | RAF inhibitor | MCOLN2 | mucolipin-2 | −0.16957 | 0.001163 | 0.028698 | −0.28064 | M | 0.190801 | M | 0.324717 |
| K05804044 | AZ-628 | RAF inhibitor | MIA | melanoma-derived growth | −0.33918 | 2.99E−11 | 1E−07 | −0.38091 | M | 0.190801 | M | 0.324717 |
| | | | | regulatory protein | | | | | | | | |
| K05804044 | AZ-628 | RAF inhibitor | NES | nestin | −0.27824 | 6.77E−08 | 3.16E−05 | −0.33874 | M | 0.190801 | M | 0.324717 |
| K05804044 | AZ-628 | RAF inhibitor | NFATC2 | nuclear factor of activated t- | −0.28168 | 4.58E−08 | 2.38E−05 | −0.37529 | M | 0.190801 | M | 0.324717 |
| | | | | cells; cytoplasmic 2 | | | | | | | | |
| K05804044 | AZ-628 | RAF inhibitor | NGFR | tumor necrosis factor | −0.17179 | 0.000999 | 0.026137 | −0.32097 | M | 0.190801 | M | 0.324717 |
| | | | | receptor superfamily | | | | | | | | |
| | | | | member 16 | | | | | | | | |
| K05804044 | AZ-628 | RAF inhibitor | NPL | n-acetylneuraminate lyase | −0.20293 | 9.66E−05 | 0.005734 | −0.24924 | M | 0.190801 | M | 0.324717 |
| K05804044 | AZ-628 | RAF inhibitor | NRROS | transforming growth factor | −0.26266 | 3.7E−07 | 0.000111 | −0.33699 | M | 0.190801 | M | 0.324717 |
| | | | | beta activator lrrc33 | | | | | | | | |
| K05804044 | AZ-628 | RAF inhibitor | PKNOX2 | homeobox protein pknox2 | −0.27144 | 1.44E−07 | 5.55E−05 | −0.32605 | M | 0.190801 | M | 0.324717 |
| K05804044 | AZ-628 | RAF inhibitor | PLP1 | myelin proteolipid protein | −0.38023 | 5.76E−14 | 1.17E−09 | −0.38754 | M | 0.190801 | M | 0.324717 |
| K05804044 | AZ-628 | RAF inhibitor | PLXNB3 | plexin-b3 | −0.22015 | 2.26E−05 | 0.002122 | −0.26462 | M | 0.190801 | M | 0.324717 |
| K05804044 | AZ-628 | RAF inhibitor | PMP2 | myelin p2 protein | −0.32496 | 2.13E−10 | 4.22E−07 | −0.36738 | M | 0.190801 | M | 0.324717 |
| K05804044 | AZ-628 | RAF inhibitor | PTCRA | pre t-cell antigen receptor | −0.33169 | 8.51E−11 | 2.15E−07 | −0.40565 | M | 0.190801 | M | 0.324717 |
| | | | | alpha | | | | | | | | |
| K05804044 | AZ-628 | RAF inhibitor | PTPRZ1 | receptor-type tyrosine- | −0.21559 | 3.36E−05 | 0.002791 | −0.31658 | M | 0.190801 | M | 0.324717 |
| | | | | protein phosphatase zeta | | | | | | | | |
| K05804044 | AZ-628 | RAF inhibitor | RENBP | n-acylglucosamine 2- | −0.22573 | 1.37E−05 | 0.001505 | −0.35896 | M | 0.190801 | M | 0.324717 |
| | | | | epimerase | | | | | | | | |
| K05804044 | AZ-628 | RAF inhibitor | RGS1 | regulator of g-protein | −0.28769 | 2.29E−08 | 1.42E−05 | −0.29038 | M | 0.190801 | M | 0.324717 |
| | | | | signaling 1 | | | | | | | | |
| K05804044 | AZ-628 | RAF inhibitor | RHOJ | rho-related gtp-binding | −0.26239 | 3.81E−07 | 0.000114 | −0.29784 | M | 0.190801 | M | 0.324717 |
| | | | | protein rhoj | | | | | | | | |
| K05804044 | AZ-628 | RAF inhibitor | SCML4 | sex comb on midleg-like | −0.27062 | 1.58E−07 | 5.93E−05 | −0.30325 | M | 0.190801 | M | 0.324717 |
| | | | | protein 4 | | | | | | | | |
| K05804044 | AZ-628 | RAF inhibitor | SCRG1 | scrapie-responsive protein 1 | −0.22836 | 1.08E−05 | 0.001268 | −0.25315 | M | 0.190801 | M | 0.324717 |
| K05804044 | AZ-628 | RAF inhibitor | SGCD | delta-sarcoglycan | −0.29116 | 1.52E−08 | 1.06E−05 | −0.31726 | M | 0.190801 | M | 0.324717 |
| K05804044 | AZ-628 | RAF inhibitor | SH3BP1 | bargin-related | −0.19178 | 0.000233 | 0.010329 | −0.35291 | M | 0.190801 | M | 0.324717 |
| K05804044 | AZ-628 | RAF inhibitor | SHC4 | shc-transforming protein 4 | −0.31312 | 1.01E−09 | 1.4E−06 | −0.3485 | M | 0.190801 | M | 0.324717 |
| K05804044 | AZ-628 | RAF inhibitor | SORCS1 | vps10 domain-containing | −0.333 | 7.11E−11 | 1.86E−07 | −0.31767 | M | 0.190801 | M | 0.324717 |
| | | | | receptor sorcs1 | | | | | | | | |
| K05804044 | AZ-628 | RAF inhibitor | SOX5 | transcription factor sox-5 | −0.24005 | 3.62E−06 | 0.00058 | −0.26172 | M | 0.190801 | M | 0.324717 |
| K05804044 | AZ-628 | RAF inhibitor | ST3GAL5 | lactosylceramide alpha-2; 3- | −0.17173 | 0.001003 | 0.026207 | −0.27252 | M | 0.190801 | M | 0.324717 |
| | | | | sialyltransferase | | | | | | | | |
| K05804044 | AZ-628 | RAF inhibitor | ST8SIA1 | alpha-n-acetylneuraminide | −0.29964 | 5.49E−09 | 4.93E−06 | −0.27099 | M | 0.190801 | M | 0.324717 |
| | | | | alpha-2; 8-sialyltransferase | | | | | | | | |
| K05804044 | AZ-628 | RAF inhibitor | STK10 | serine/threonine-protein | −0.16439 | 0.001649 | 0.035446 | −0.26021 | M | 0.190801 | M | 0.324717 |
| | | | | kinase 10 | | | | | | | | |
| K05804044 | AZ-628 | RAF inhibitor | TAMALIN | protein tamalin | −0.23971 | 3.74E−06 | 0.000593 | −0.24244 | M | 0.190801 | M | 0.324717 |
| K05804044 | AZ-628 | RAF inhibitor | TIMP3 | metalloproteinase inhibitor 3 | −0.21966 | 2.35E−05 | 0.00218 | −0.28658 | M | 0.190801 | M | 0.324717 |
| K05804044 | AZ-628 | RAF inhibitor | TMEM229B | transmembrane protein 229b | −0.21657 | 3.08E−05 | 0.002632 | −0.30604 | M | 0.190801 | M | 0.324717 |
| K05804044 | AZ-628 | RAF inhibitor | TNFRSF14 | tumor necrosis factor | −0.26507 | 2.87E−07 | 9.23E−05 | −0.32016 | M | 0.190801 | M | 0.324717 |
| | | | | receptor superfamily | | | | | | | | |
| | | | | member 14 | | | | | | | | |
| K05804044 | AZ-628 | RAF inhibitor | TRPV2 | transient receptor potential | −0.35718 | 2.16E−12 | 1.45E−08 | −0.34489 | M | 0.190801 | M | 0.324717 |
| | | | | cation channel subfamily v | | | | | | | | |
| | | | | member 2 | | | | | | | | |
| K05804044 | AZ-628 | RAF inhibitor | WFDC1 | wap four-disulfide core | −0.21807 | 2.71E−05 | 0.002404 | −0.30701 | M | 0.190801 | M | 0.324717 |
| | | | | domain protein 1 | | | | | | | | |
| K07106112 | BMS-599626 | EGFR inhibitor; protein | ADAM8 | disintegrin and | −0.16357 | 0.001742 | 0.036642 | −0.2475 | IM | −0.07697 | IM | −0.02871 |
| | | tyrosine kinase inhibitor | | metalloproteinase domain- | | | | | | | | |
| | | | | containing protein 8 | | | | | | | | |
| K07106112 | BMS-599626 | EGFR inhibitor; protein | ALOX5 | polyunsaturated fatty acid 5- | −0.18003 | 0.000558 | 0.018103 | −0.39479 | IM | −0.07697 | IM | −0.02871 |
| | | tyrosine kinase inhibitor | | lipoxygenase | | | | | | | | |
| K07106112 | BMS-599626 | EGFR inhibitor; protein | ANKRD65 | ankyrin repeat domain- | −0.24397 | 2.48E−06 | 0.000439 | −0.30311 | IM | −0.07697 | IM | −0.02871 |
| | | tyrosine kinase inhibitor | | containing protein 65 | | | | | | | | |
| K07106112 | BMS-599626 | EGFR inhibitor; protein | COL17A1 | collagen alpha-1 | −0.31878 | 4.84E−10 | 7.91E−07 | −0.38241 | IM | −0.07697 | IM | −0.02871 |
| | | tyrosine kinase inhibitor | | | | | | | | | | |
| K07106112 | BMS-599626 | EGFR inhibitor; protein | CXCL16 | c-x-c motif chemokine 16 | −0.25418 | 8.94E−07 | 0.000211 | −0.30013 | IM | −0.07697 | IM | −0.02871 |
| | | tyrosine kinase inhibitor | | | | | | | | | | |
| K07106112 | BMS-599626 | EGFR inhibitor; protein | CYP4B1 | cytochrome p450 4b1 | −0.18372 | 0.000426 | 0.015278 | −0.36885 | IM | −0.07697 | IM | −0.02871 |
| | | tyrosine kinase inhibitor | | | | | | | | | | |
| K07106112 | BMS-599626 | EGFR inhibitor; protein | DAPP1 | dual adapter for | −0.32404 | 2.41E−10 | 4.68E−07 | −0.42856 | IM | −0.07697 | IM | −0.02871 |
| | | tyrosine kinase inhibitor | | phosphotyrosine and 3- | | | | | | | | |
| | | | | phosphotyrosine and 3- | | | | | | | | |
| | | | | phosphoinositide | | | | | | | | |
| K07106112 | BMS-599626 | EGFR inhibitor; protein | DENND1C | denn domain-containing | −0.30086 | 4.73E−09 | 4.4E−06 | −0.37138 | IM | −0.07697 | IM | −0.02871 |
| | | tyrosine kinase inhibitor | | protein 1c | | | | | | | | |
| K07106112 | BMS-599626 | EGFR inhibitor; protein | DENND2D | denn domain-containing | −0.21421 | 3.78E−05 | 0.003028 | −0.28809 | IM | −0.07697 | IM | −0.02871 |
| | | tyrosine kinase inhibitor | | protein 2d | | | | | | | | |
| K07106112 | BMS-599626 | EGFR inhibitor; protein | DSG3 | desmoglein-3 | −0.37826 | 7.94E−14 | 1.4E−09 | −0.27403 | IM | −0.07697 | IM | −0.02871 |
| | | tyrosine kinase inhibitor | | | | | | | | | | |
| K07106112 | BMS-599626 | EGFR inhibitor; protein | FGD3 | fyve; rhogef and ph domain- | −0.29471 | 9.98E−09 | 7.72E−06 | −0.40975 | IM | −0.07697 | IM | −0.02871 |
| | | tyrosine kinase inhibitor | | containing protein 3 | | | | | | | | |
| K07106112 | BMS-599626 | EGFR inhibitor; protein | HSH2D | hematopoietic sh2 domain- | −0.18696 | 0.000335 | 0.013102 | −0.37526 | IM | −0.07697 | IM | −0.02871 |
| | | tyrosine kinase inhibitor | | containing protein | | | | | | | | |
| K07106112 | BMS-599626 | EGFR inhibitor; protein | ITGB4 | integrin beta-4 | −0.36154 | 1.11E−12 | 8.77E−09 | −0.45377 | IM | −0.07697 | IM | −0.02871 |
| | | tyrosine kinase inhibitor | | | | | | | | | | |
| K07106112 | BMS-599626 | EGFR inhibitor; protein | KRT16 | keratin; type i cytoskeletal | −0.36363 | 8.05E−13 | 7.04E−09 | −0.36269 | IM | −0.07697 | IM | −0.02871 |
| | | tyrosine kinase inhibitor | | 16 | | | | | | | | |
| K07106112 | BMS-599626 | EGFR inhibitor; protein | KRT17 | keratin; type i cytoskeletal | −0.34243 | 1.88E−11 | 7.13E−08 | −0.32754 | IM | −0.07697 | IM | −0.02871 |
| | | tyrosine kinase inhibitor | | 17 | | | | | | | | |
| K07106112 | BMS-599626 | EGFR inhibitor; protein | KRT5 | keratin; type ii cytoskeletal | −0.3821 | 4.24E−14 | 9.54E−10 | −0.33086 | IM | −0.07697 | IM | −0.02871 |
| | | tyrosine kinase inhibitor | | 5 | | | | | | | | |
| K07106112 | BMS-599626 | EGFR inhibitor; protein | LGALS9 | galectin-9 | −0.19215 | 0.000226 | 0.010143 | −0.25463 | IM | −0.07697 | IM | −0.02871 |
| | | tyrosine kinase inhibitor | | | | | | | | | | |
| K07106112 | BMS-599626 | EGFR inhibitor; protein | LPAR5 | lysophosphatidic acid | −0.2942 | 1.06E−08 | 8.07E−06 | −0.32056 | IM | −0.07697 | IM | −0.02871 |
| | | tyrosine kinase inhibitor | | receptor 5 | | | | | | | | |
| K07106112 | BMS-599626 | EGFR inhibitor; protein | MFNG | beta-1; 3-n- | −0.25803 | 6.01E−07 | 0.000159 | −0.26797 | IM | −0.07697 | IM | −0.02871 |
| | | tyrosine kinase inhibitor | | acetylglucosaminyltransferase | | | | | | | | |
| | | | | manic fringe | | | | | | | | |
| K07106112 | BMS-599626 | EGFR inhibitor; protein | PTAFR | platelet-activating factor | −0.32355 | 2.57E−10 | 4.89E−07 | −0.45066 | IM | −0.07697 | IM | −0.02871 |
| | | tyrosine kinase inhibitor | | receptor | | | | | | | | |
| K07106112 | BMS-599626 | EGFR inhibitor; protein | PTPN6 | tyrosine-protein phosphatase | −0.3556 | 2.73E−12 | 1.68E−08 | −0.42466 | IM | −0.07697 | IM | −0.02871 |
| | | tyrosine kinase inhibitor | | non-receptor type 6 | | | | | | | | |
| K07106112 | BMS-599626 | EGFR inhibitor; protein | S100A8 | protein s100-a8 | −0.25994 | 4.93E−07 | 0.000137 | −0.26807 | IM | −0.07697 | IM | −0.02871 |
| | | tyrosine kinase inhibitor | | | | | | | | | | |
| K07106112 | BMS-599626 | EGFR inhibitor; protein | SH3BP1 | bargin-related | −0.26612 | 2.56E−07 | 8.52E−05 | −0.29862 | IM | −0.07697 | IM | −0.02871 |
| | | tyrosine kinase inhibitor | | | | | | | | | | |
| K07106112 | BMS-599626 | EGFR inhibitor; protein | TEAD3 | transcriptional enhancer | −0.26609 | 2.57E−07 | 8.54E−05 | −0.25365 | IM | −0.07697 | IM | −0.02871 |
| | | tyrosine kinase inhibitor | | factor tef-5 | | | | | | | | |
| K07106112 | BMS-599626 | EGFR inhibitor; protein | TNFAIP8 | tumor necrosis factor alpha- | −0.19633 | 0.000164 | 0.008171 | −0.23935 | IM | −0.07697 | IM | −0.02871 |
| | | tyrosine kinase inhibitor | | induced protein 8 | | | | | | | | |
| K07106112 | BMS-599626 | EGFR inhibitor; protein | TNFSF10 | tumor necrosis factor ligand | −0.29818 | 6.56E−09 | 5.64E−06 | −0.33751 | IM | −0.07697 | IM | −0.02871 |
| | | tyrosine kinase inhibitor | | superfamily member 10 | | | | | | | | |
| K07106112 | BMS-599626 | EGFR inhibitor; protein | UNC13D | protein unc-13 homolog d | −0.17233 | 0.000963 | 0.025528 | −0.3177 | IM | −0.07697 | IM | −0.02871 |
| | | tyrosine kinase inhibitor | | | | | | | | | | |
| K07106112 | BMS-599626 | EGFR inhibitor; protein | VIPR1 | vasoactive intestinal | −0.19563 | 0.000173 | 0.008489 | −0.31868 | IM | −0.07697 | IM | −0.02871 |
| | | tyrosine kinase inhibitor | | polypeptide receptor 1 | | | | | | | | |
| K09416995 | lovastatin | HMGCR inhibitor | ANGPTL7 | angiopoietin-related protein 7 | −0.19501 | 0.000185 | 0.008874 | −0.23758 | M | 0.051166 | M | 0.05706 |
| K09416995 | lovastatin | HMGCR inhibitor | ARMCX1 | armadillo repeat-containing | −0.16082 | 0.002116 | 0.041176 | −0.26346 | M | 0.051166 | M | 0.05706 |
| | | | | x-linked protein 1 | | | | | | | | |
| K09416995 | lovastatin | HMGCR inhibitor | CSPG4 | chondroitin sulfate | −0.15944 | 0.002313 | 0.043375 | −0.31573 | M | 0.051166 | M | 0.05706 |
| | | | | proteoglycan 4 | | | | | | | | |
| K09416995 | lovastatin | HMGCR inhibitor | CYBRD1 | cytochrome b reductase 1 | −0.19789 | 0.000148 | 0.007636 | −0.26214 | M | 0.051166 | M | 0.05706 |
| K09416995 | lovastatin | HMGCR inhibitor | EPHA3 | ephrin type-a receptor 3 | −0.21134 | 4.94E−05 | 0.00364 | −0.257 | M | 0.051166 | M | 0.05706 |
| K09416995 | lovastatin | HMGCR inhibitor | EVC | ellis-van creveld syndrome | −0.17333 | 0.000912 | 0.024666 | −0.25736 | M | 0.051166 | M | 0.05706 |
| | | | | protein | | | | | | | | |
| K09416995 | lovastatin | HMGCR inhibitor | FGL2 | fibroleukin | −0.1825 | 0.000475 | 0.016369 | −0.26056 | M | 0.051166 | M | 0.05706 |
| K09416995 | lovastatin | HMGCR inhibitor | MMP16 | matrix metalloproteinase-16 | −0.19006 | 0.000271 | 0.01139 | −0.26083 | M | 0.051166 | M | 0.05706 |
| K09416995 | lovastatin | HMGCR inhibitor | PDE1C | calcium/calmodulin- | −0.22554 | 1.44E−05 | 0.001555 | −0.29327 | M | 0.051166 | M | 0.05706 |
| | | | | dependent 3′; 5′-cyclic | | | | | | | | |
| | | | | nucleotide | | | | | | | | |
| | | | | phosphodiesterase 1c | | | | | | | | |
| K09416995 | lovastatin | HMGCR inhibitor | PTN | pleiotrophin | −0.20096 | 0.000116 | 0.006479 | −0.23928 | M | 0.051166 | M | 0.05706 |
| K09416995 | lovastatin | HMGCR inhibitor | RASSF8 | ras association domain- | −0.17504 | 0.00081 | 0.022883 | −0.25575 | M | 0.051166 | M | 0.05706 |
| | | | | containing protein 8 | | | | | | | | |
| K09416995 | lovastatin | HMGCR inhibitor | REEP2 | receptor expression- | −0.16027 | 0.002192 | 0.04206 | −0.26037 | M | 0.051166 | M | 0.05706 |
| | | | | enhancing protein 2 | | | | | | | | |
| K09416995 | lovastatin | HMGCR inhibitor | RHOJ | rho-related gtp-binding | −0.24636 | 2.02E−06 | 0.00038 | −0.31485 | M | 0.051166 | M | 0.05706 |
| | | | | protein rhoj | | | | | | | | |
| K09416995 | lovastatin | HMGCR inhibitor | SCRG1 | scrapie-responsive protein 1 | −0.24725 | 1.85E−06 | 0.000358 | −0.24415 | M | 0.051166 | M | 0.05706 |
| K09416995 | lovastatin | HMGCR inhibitor | SPARC | sparc | −0.19645 | 0.000165 | 0.008241 | −0.25228 | M | 0.051166 | M | 0.05706 |
| K09416995 | lovastatin | HMGCR inhibitor | SRPX | sushi repeat-containing | −0.25233 | 1.12E−06 | 0.000249 | −0.23894 | M | 0.051166 | M | 0.05706 |
| | | | | protein srpx | | | | | | | | |
| K09416995 | lovastatin | HMGCR inhibitor | SYDE1 | rho gtpase-activating protein | −0.23875 | 4.23E−06 | 0.000644 | −0.26759 | M | 0.051166 | M | 0.05706 |
| | | | | sydel | | | | | | | | |
| K09416995 | lovastatin | HMGCR inhibitor | TIMP2 | metalloproteinase inhibitor 2 | −0.18333 | 0.000447 | 0.015746 | −0.24673 | M | 0.051166 | M | 0.05706 |
| K09416995 | lovastatin | HMGCR inhibitor | TMEM255A | transmembrane protein 255a | −0.15727 | 0.002657 | 0.047066 | −0.29772 | M | 0.051166 | M | 0.05706 |
| K09416995 | lovastatin | HMGCR inhibitor | VIM | vimentin | −0.20599 | 7.7E−05 | 0.004925 | −0.25451 | M | 0.051166 | M | 0.05706 |
| K13060017 | UNC0631 | histone lysine | CHL1 | atp-dependent dna helicase | −0.19048 | 0.000326 | 0.012863 | −0.24128 | M | 0.105455 | M | 0.163017 |
| | | methyltransferase | | ddx11-related | | | | | | | | |
| | | inhibitor | | | | | | | | | | |
| K13060017 | UNC0631 | histone lysine | COL19A1 | collagen alpha-1 | −0.25277 | 1.56E−06 | 0.000315 | −0.2431 | M | 0.105455 | M | 0.163017 |
| | | methyltransferase | | | | | | | | | | |
| | | inhibitor | | | | | | | | | | |
| K13060017 | UNC0631 | histone lysine | CSPG4 | chondroitin sulfate | −0.15961 | 0.002673 | 0.047237 | −0.29838 | M | 0.105455 | M | 0.163017 |
| | | methyltransferase | | proteoglycan 4 | | | | | | | | |
| | | inhibitor | | | | | | | | | | |
| K13060017 | UNC0631 | histone lysine | CST7 | cystatin-f | −0.17664 | 0.000873 | 0.023992 | −0.33144 | M | 0.105455 | M | 0.163017 |
| | | methyltransferase | | | | | | | | | | |
| | | inhibitor | | | | | | | | | | |
| K13060017 | UNC0631 | histone lysine | DAAM2 | disheveled-associated | −0.16242 | 0.002238 | 0.042566 | −0.3327 | M | 0.105455 | M | 0.163017 |
| | | methyltransferase | | activator of morphogenesis | | | | | | | | |
| | | inhibitor | | 2 | | | | | | | | |
| K13060017 | UNC0631 | histone lysine | EDNRB | endothelin receptor type b | −0.20035 | 0.000154 | 0.007863 | −0.27429 | M | 0.105455 | M | 0.163017 |
| | | methyltransferase | | | | | | | | | | |
| | | inhibitor | | | | | | | | | | |
| K13060017 | UNC0631 | histone lysine | GNG2 | guanine nucleotide-binding | −0.22329 | 2.36E−05 | 0.002182 | −0.31662 | M | 0.105455 | M | 0.163017 |
| | | methyltransferase | | protein g | | | | | | | | |
| | | inhibitor | | | | | | | | | | |
| K13060017 | UNC0631 | histone lysine | HMCN1 | hemicentin-1 | −0.16061 | 0.00251 | 0.045491 | −0.33456 | M | 0.105455 | M | 0.163017 |
| | | methyltransferase | | | | | | | | | | |
| | | inhibitor | | | | | | | | | | |
| K13060017 | UNC0631 | histone lysine | IL16 | pro-interleukin-16 | −0.21748 | 3.87E−05 | 0.003076 | −0.31353 | M | 0.105455 | M | 0.163017 |
| | | methyltransferase | | | | | | | | | | |
| | | inhibitor | | | | | | | | | | |
| K13060017 | UNC0631 | histone lysine | LSAMP | limbic system-associated | −0.16546 | 0.00184 | 0.037877 | −0.26388 | M | 0.105455 | M | 0.163017 |
| | | methyltransferase | | membrane protein | | | | | | | | |
| | | inhibitor | | | | | | | | | | |
| K13060017 | UNC0631 | histone lysine | LZTS1 | | −0.16602 | 0.001775 | 0.037078 | −0.2903 | M | 0.105455 | M | 0.163017 |
| | | methyltransferase | | | | | | | | | | |
| | | inhibitor | | | | | | | | | | |
| K13060017 | UNC0631 | histone lysine | NGFR | tumor necrosis factor | −0.17863 | 0.000761 | 0.021993 | −0.31638 | M | 0.105455 | M | 0.163017 |
| | | methyltransferase | | receptor superfamily | | | | | | | | |
| | | inhibitor | | member 16 | | | | | | | | |
| K13060017 | UNC0631 | histone lysine | P2RX7 | p2x purinoceptor 7 | −0.22645 | 1.79E−05 | 0.001808 | −0.30062 | M | 0.105455 | M | 0.163017 |
| | | methyltransferase | | | | | | | | | | |
| | | inhibitor | | | | | | | | | | |
| K13060017 | UNC0631 | histone lysine | PLEKHO2 | pleckstrin homology | −0.17007 | 0.001361 | 0.03155 | −0.30906 | M | 0.105455 | M | 0.163017 |
| | | methyltransferase | | domain-containing family o | | | | | | | | |
| | | inhibitor | | member 2 | | | | | | | | |
| K13060017 | UNC0631 | histone lysine | PLP1 | myelin proteolipid protein | −0.21155 | 6.33E−05 | 0.004314 | −0.26933 | M | 0.105455 | M | 0.163017 |
| | | methyltransferase | | | | | | | | | | |
| | | inhibitor | | | | | | | | | | |
| K13060017 | UNC0631 | histone lysine | PLXNB3 | plexin-b3 | −0.20018 | 0.000156 | 0.007925 | −0.24736 | M | 0.105455 | M | 0.163017 |
| | | methyltransferase | | | | | | | | | | |
| | | inhibitor | | | | | | | | | | |
| K13060017 | UNC0631 | histone lysine | RCN2 | reticulocalbin-2 | −0.17331 | 0.001095 | 0.027688 | −0.29837 | M | 0.105455 | M | 0.163017 |
| | | methyltransferase | | | | | | | | | | |
| | | inhibitor | | | | | | | | | | |
| K13060017 | UNC0631 | histone lysine | SGCD | delta-sarcoglycan | −0.15902 | 0.002772 | 0.048275 | −0.291 | M | 0.105455 | M | 0.163017 |
| | | methyltransferase | | | | | | | | | | |
| | | inhibitor | | | | | | | | | | |
| K13060017 | UNC0631 | histone lysine | SRPX | sushi repeat-containing | −0.16955 | 0.001409 | 0.032234 | −0.33386 | M | 0.105455 | M | 0.163017 |
| | | methyltransferase | | protein srpx | | | | | | | | |
| | | inhibitor | | | | | | | | | | |
| K13060017 | UNC0631 | histone lysine | ST8SIA1 | alpha-n-acetylneuraminide | −0.22286 | 2.45E−05 | 0.002242 | −0.24416 | M | 0.105455 | M | 0.163017 |
| | | methyltransferase | | alpha-2; 8-sialy ltransferase | | | | | | | | |
| | | inhibitor | | | | | | | | | | |
| K13060017 | UNC0631 | histone lysine | TRPV2 | transient receptor potential | −0.25497 | 1.25E−06 | 0.00027 | −0.26572 | M | 0.105455 | M | 0.163017 |
| | | methyltransferase | | cation channel subfamily v | | | | | | | | |
| | | inhibitor | | member 2 | | | | | | | | |
| K13183738 | pentamidine | anti-pneumocystis agent | ACACB | acetyl-coa carboxylase 2 | −0.15713 | 0.002646 | 0.046937 | −0.26193 | M | 0.215745 | M | 0.139961 |
| K13183738 | pentamidine | anti-pneumocystis agent | ADAM11 | disintegrin and | −0.19946 | 0.000128 | 0.006925 | −0.27135 | M | 0.215745 | M | 0.139961 |
| | | | | metalloproteinase domain- | | | | | | | | |
| | | | | containing protein 11 | | | | | | | | |
| K13183738 | pentamidine | anti-pneumocystis agent | DVL2 | segment polarity protein | −0.19711 | 0.000154 | 0.007843 | −0.30089 | M | 0.215745 | M | 0.139961 |
| | | | | dishevelled homolog dvl-2 | | | | | | | | |
| K13183738 | pentamidine | anti-pneumocystis agent | JAM3 | junctional adhesion | −0.15815 | 0.002478 | 0.045153 | −0.24591 | M | 0.215745 | M | 0.139961 |
| | | | | molecule c | | | | | | | | |
| K13183738 | pentamidine | anti-pneumocystis agent | MPP2 | forkhead box protein ml | −0.16459 | 0.001628 | 0.035152 | −0.28632 | M | 0.215745 | M | 0.139961 |
| K13183738 | pentamidine | anti-pneumocystis agent | PDZD4 | pdz domain-containing | −0.20438 | 8.58E−05 | 0.005296 | −0.26962 | M | 0.215745 | M | 0.139961 |
| | | | | protein 4 | | | | | | | | |
| K13183738 | pentamidine | anti-pneumocystis agent | POLR2A | dna-directed rna polymerase | −0.15586 | 0.002867 | 0.04925 | −0.3033 | M | 0.215745 | M | 0.139961 |
| | | | | ii subunit rpb1 | | | | | | | | |
| K13183738 | pentamidine | anti-pneumocystis agent | SMO | smoothened homolog | −0.27546 | 9.24E−08 | 3.98E−05 | −0.2583 | M | 0.215745 | M | 0.139961 |
| K13183738 | pentamidine | anti-pneumocystis agent | TAMALIN | protein tamalin | −0.17156 | 0.001015 | 0.026407 | −0.25341 | M | 0.215745 | M | 0.139961 |
| K13183738 | pentamidine | anti-pneumocystis agent | TUB | tubby protein homolog | −0.27417 | 1.07E−07 | 4.46E−05 | −0.26021 | M | 0.215745 | M | 0.139961 |
| K16180792 | bis(maltolato)oxo- | tyrosine phosphatase | ADAMTS1 | a disintegrin and | −0.19165 | 0.000299 | 0.012145 | −0.31976 | M | 0.148517 | M | 0.090307 |
| | vanadium(IV) | inhibitor | | metalloproteinase with | | | | | | | | |
| | | | | thrombospondin motifs 1 | | | | | | | | |
| K16180792 | bis(maltolato)oxo- | tyrosine phosphatase | ADAMTS9 | a disintegrin and | −0.31119 | 2.43E−09 | 2.69E−06 | −0.31362 | M | 0.148517 | M | 0.090307 |
| | vanadium(IV) | inhibitor | | metalloproteinase with | | | | | | | | |
| | | | | thrombospondin motifs 9 | | | | | | | | |
| K16180792 | bis(maltolato)oxo- | tyrosine phosphatase | ANKRD44 | serine/threonine-protein | −0.20261 | 0.000129 | 0.006982 | −0.28974 | M | 0.148517 | M | 0.090307 |
| | vanadium(IV) | inhibitor | | phosphatase 6 regulatory | | | | | | | | |
| | | | | ankyrin repeat subunit b | | | | | | | | |
| K16180792 | bis(maltolato)oxo- | tyrosine phosphatase | ANTXR1 | anthrax toxin receptor 1 | −0.2193 | 3.32E−05 | 0.002769 | −0.27465 | M | 0.148517 | M | 0.090307 |
| | vanadium(IV) | inhibitor | | | | | | | | | | |
| K16180792 | bis(maltolato)oxo- | tyrosine phosphatase | ARHGAP31 | rho gtpase-activating protein | −0.21331 | 5.48E−05 | 0.003904 | −0.28188 | M | 0.148517 | M | 0.090307 |
| | vanadium(IV) | inhibitor | | 31 | | | | | | | | |
| K16180792 | bis(maltolato)oxo- | tyrosine phosphatase | BCL2A1 | bcl-2-related protein a1 | −0.19582 | 0.000218 | 0.009928 | −0.31153 | M | 0.148517 | M | 0.090307 |
| | vanadium(IV) | inhibitor | | | | | | | | | | |
| K16180792 | bis(maltolato)oxo- | tyrosine phosphatase | C3AR1 | c3a anaphylatoxin | −0.18512 | 0.000481 | 0.0165 | −0.31536 | M | 0.148517 | M | 0.090307 |
| | vanadium(IV) | inhibitor | | chemotactic receptor | | | | | | | | |
| K16180792 | bis(maltolato)oxo- | tyrosine phosphatase | CALD1 | caldesmon | −0.2835 | 6.25E−08 | 2.98E−05 | −0.32385 | M | 0.148517 | M | 0.090307 |
| | vanadium(IV) | inhibitor | | | | | | | | | | |
| K16180792 | bis(maltolato)oxo- | tyrosine phosphatase | CCN2 | con family member 2 | −0.18819 | 0.000385 | 0.014297 | −0.25238 | M | 0.148517 | M | 0.090307 |
| | vanadium(IV) | inhibitor | | | | | | | | | | |
| K16180792 | bis(maltolato)oxo- | tyrosine phosphatase | CD96 | t-cell surface protein tactile | −0.29422 | 1.85E−08 | 1.22E−05 | −0.37879 | M | 0.148517 | M | 0.090307 |
| | vanadium(IV) | inhibitor | | | | | | | | | | |
| K16180792 | bis(maltolato)oxo- | tyrosine phosphatase | CDH19 | cadherin-19 | −0.28672 | 4.36E−08 | 2.29E−05 | −0.31266 | M | 0.148517 | M | 0.090307 |
| | vanadium(IV) | inhibitor | | | | | | | | | | |
| K16180792 | bis(maltolato)oxo- | tyrosine phosphatase | CERKL | ceramide kinase-like protein | −0.20404 | 0.000116 | 0.006479 | −0.28662 | M | 0.148517 | M | 0.090307 |
| | vanadium(IV) | inhibitor | | | | | | | | | | |
| K16180792 | bis(maltolato)oxo- | tyrosine phosphatase | COL19A1 | collagen alpha-1 | −0.26962 | 2.81E−07 | 9.1E−05 | −0.29878 | M | 0.148517 | M | 0.090307 |
| | vanadium(IV) | inhibitor | | | | | | | | | | |
| K16180792 | bis(maltolato)oxo- | tyrosine phosphatase | COL4A2 | collagen alpha-2 | −0.18956 | 0.000349 | 0.013431 | −0.31187 | M | 0.148517 | M | 0.090307 |
| | vanadium(IV) | inhibitor | | | | | | | | | | |
| K16180792 | bis(maltolato)oxo- | tyrosine phosphatase | CPQ | carboxypeptidase q | −0.28128 | 7.99E−08 | 3.58E−05 | −0.29139 | M | 0.148517 | M | 0.090307 |
| | vanadium(IV) | inhibitor | | | | | | | | | | |
| K16180792 | bis(maltolato)oxo- | tyrosine phosphatase | CSPG4 | chondroitin sulfate | −0.17729 | 0.000835 | 0.023313 | −0.29778 | M | 0.148517 | M | 0.090307 |
| | vanadium(IV) | inhibitor | | proteoglycan 4 | | | | | | | | |
| K16180792 | bis(maltolato)oxo- | tyrosine phosphatase | CTLA4 | cytotoxic t-lymphocyte | −0.22705 | 1.7E−05 | 0.001747 | −0.24734 | M | 0.148517 | M | 0.090307 |
| | vanadium(IV) | inhibitor | | protein 4 | | | | | | | | |
| K16180792 | bis(maltolato)oxo- | tyrosine phosphatase | DAAM2 | disheveled-associated | −0.18851 | 0.000376 | 0.0141 | −0.35354 | M | 0.148517 | M | 0.090307 |
| | vanadium(IV) | inhibitor | | activator of morphogenesis 2 | | | | | | | | |
| K16180792 | bis(maltolato)oxo- | tyrosine phosphatase | DLC1 | rho gtpase-activating protein | −0.29197 | 2.4E−08 | 1.47E−05 | −0.24823 | M | 0.148517 | M | 0.090307 |
| | vanadium(IV) | inhibitor | | 7 | | | | | | | | |
| K16180792 | bis(maltolato)oxo- | tyrosine phosphatase | DRAM1 | dna damage-regulated | −0.17328 | 0.001098 | 0.027735 | −0.27887 | M | 0.148517 | M | 0.090307 |
| | vanadium(IV) | inhibitor | | autophagy modulator | | | | | | | | |
| | | | | protein 1 | | | | | | | | |
| K16180792 | bis(maltolato)oxo- | tyrosine phosphatase | EDNRB | endothelin receptor type b | −0.25011 | 2.02E−06 | 0.00038 | −0.24971 | M | 0.148517 | M | 0.090307 |
| | vanadium(IV) | inhibitor | | | | | | | | | | |
| K16180792 | bis(maltolato)oxo- | tyrosine phosphatase | EPHA3 | ephrin type-a receptor 3 | −0.17155 | 0.001233 | 0.029726 | −0.2452 | M | 0.148517 | M | 0.090307 |
| | vanadium(IV) | inhibitor | | | | | | | | | | |
| K16180792 | bis(maltolato)oxo- | tyrosine phosphatase | FCGR2A | low affinity | −0.28126 | 8.01E−08 | 3.59E−05 | −0.2796 | M | 0.148517 | M | 0.090307 |
| | vanadium(IV) | inhibitor | | immunoglobulin gamma fc | | | | | | | | |
| | | | | region receptor ii-a-related | | | | | | | | |
| K16180792 | bis(maltolato)oxo- | tyrosine phosphatase | FCRLA | fc receptor-like a | −0.2598 | 7.73E−07 | 0.00019 | −0.32843 | M | 0.148517 | M | 0.090307 |
| | vanadium(IV) | inhibitor | | | | | | | | | | |
| K16180792 | bis(maltolato)oxo- | tyrosine phosphatase | FERMT2 | fermitin family homolog 2 | −0.24825 | 2.42E−06 | 0.00043 | −0.26591 | M | 0.148517 | M | 0.090307 |
| | vanadium(IV) | inhibitor | | | | | | | | | | |
| K16180792 | bis(maltolato)oxo- | tyrosine phosphatase | FKBP7 | peptidyl-prolyl cis-trans | −0.21984 | 3.17E−05 | 0.002684 | −0.24966 | M | 0.148517 | M | 0.090307 |
| | vanadium(IV) | inhibitor | | isomerase fkbp7 | | | | | | | | |
| K16180792 | bis(maltolato)oxo- | tyrosine phosphatase | FLT1 | vascular endothelial growth | −0.17472 | 0.000995 | 0.026069 | −0.31053 | M | 0.148517 | M | 0.090307 |
| | vanadium(IV) | inhibitor | | factor receptor 1 | | | | | | | | |
| K16180792 | bis(maltolato)oxo- | tyrosine phosphatase | FN1 | fibronectin | −0.27474 | 1.63E−07 | 6.07E−05 | −0.26405 | M | 0.148517 | M | 0.090307 |
| | vanadium(IV) | inhibitor | | | | | | | | | | |
| K16180792 | bis(maltolato)oxo- | tyrosine phosphatase | GAS7 | growth arrest-specific | −0.25575 | 1.16E−06 | 0.000256 | −0.28382 | M | 0.148517 | M | 0.090307 |
| | vanadium(IV) | inhibitor | | protein 7 | | | | | | | | |
| K16180792 | bis(maltolato)oxo- | tyrosine phosphatase | GDNF | glial cell line-derived | −0.19768 | 0.00019 | 0.009029 | −0.28533 | M | 0.148517 | M | 0.090307 |
| | vanadium(IV) | inhibitor | | neurotrophic factor | | | | | | | | |
| K16180792 | bis(maltolato)oxo- | tyrosine phosphatase | GYPC | glycophorin-c | −0.23894 | 5.82E−06 | 0.00081 | −0.27453 | M | 0.148517 | M | 0.090307 |
| | vanadium(IV) | inhibitor | | | | | | | | | | |
| K16180792 | bis(maltolato)oxo- | tyrosine phosphatase | HEG1 | protein heg homolog 1 | −0.17654 | 0.000879 | 0.024099 | −0.25485 | M | 0.148517 | M | 0.090307 |
| | vanadium(IV) | inhibitor | | | | | | | | | | |
| K16180792 | bis(maltolato)oxo- | tyrosine phosphatase | HPS5 | hermansky-pudlak | −0.2768 | 1.3E−07 | 5.17E−05 | −0.35554 | M | 0.148517 | M | 0.090307 |
| | vanadium(IV) | inhibitor | | syndrome 5 protein | | | | | | | | |
| K16180792 | bis(maltolato)oxo- | tyrosine phosphatase | IGFBP7 | insulin-like growth factor- | −0.23741 | 6.7E−06 | 0.000898 | −0.25232 | M | 0.148517 | M | 0.090307 |
| | vanadium(IV) | inhibitor | | binding protein 7 | | | | | | | | |
| K16180792 | bis(maltolato)oxo- | tyrosine phosphatase | ITGA4 | integrin alpha-4 | −0.19276 | 0.000275 | 0.011515 | −0.27254 | M | 0.148517 | M | 0.090307 |
| | vanadium(IV) | inhibitor | | | | | | | | | | |
| K16180792 | bis(maltolato)oxo- | tyrosine phosphatase | ITGB3 | integrin beta-3 | −0.41144 | 8.19E−16 | 4.61E−11 | −0.42256 | M | 0.148517 | M | 0.090307 |
| | vanadium(IV) | inhibitor | | | | | | | | | | |
| K16180792 | bis(maltolato)oxo- | tyrosine phosphatase | KIRREL1 | kin of irre-like protein 1 | −0.29106 | 2.66E−08 | 1.59E−05 | −0.41603 | M | 0.148517 | M | 0.090307 |
| | vanadium(IV) | inhibitor | | | | | | | | | | |
| K16180792 | bis(maltolato)oxo- | tyrosine phosphatase | KLHL29 | kelch-like protein 29 | −0.16538 | 0.001851 | 0.038 | −0.26831 | M | 0.148517 | M | 0.090307 |
| | vanadium(IV) | inhibitor | | | | | | | | | | |
| K16180792 | bis(maltolato)oxo- | tyrosine phosphatase | KRBA1 | protein krbal | −0.22422 | 2.18E−05 | 0.002066 | −0.2633 | M | 0.148517 | M | 0.090307 |
| | vanadium(IV) | inhibitor | | | | | | | | | | |
| K16180792 | bis(maltolato)oxo- | tyrosine phosphatase | LAMA4 | laminin subunit alpha-4 | −0.19623 | 0.000212 | 0.009724 | −0.2547 | M | 0.148517 | M | 0.090307 |
| | vanadium(IV) | inhibitor | | | | | | | | | | |
| K16180792 | bis(maltolato)oxo- | tyrosine phosphatase | LARP6 | la-related protein 6 | −0.18912 | 0.00036 | 0.013714 | −0.25689 | M | 0.148517 | M | 0.090307 |
| | vanadium(IV) | inhibitor | | | | | | | | | | |
| K16180792 | bis(maltolato)oxo- | tyrosine phosphatase | LZTS1 | | −0.26537 | 4.37E−07 | 0.000125 | −0.30303 | M | 0.148517 | M | 0.090307 |
| | vanadium(IV) | inhibitor | | | | | | | | | | |
| K16180792 | bis(maltolato)oxo- | tyrosine phosphatase | MCAM | cell surface glycoprotein | −0.22147 | 2.76E−05 | 0.002434 | −0.3214 | M | 0.148517 | M | 0.090307 |
| | vanadium(IV) | inhibitor | | muc18 | | | | | | | | |
| K16180792 | bis(maltolato)oxo- | tyrosine phosphatase | MFGE8 | lactadherin | −0.18261 | 0.000576 | 0.018465 | −0.31014 | M | 0.148517 | M | 0.090307 |
| | vanadium(IV) | inhibitor | | | | | | | | | | |
| K16180792 | bis(maltolato)oxo- | tyrosine phosphatase | MIA | melanoma-derived growth | −0.20592 | 9.96E−05 | 0.005855 | −0.29543 | M | 0.148517 | M | 0.090307 |
| | vanadium(IV) | inhibitor | | regulatory protein | | | | | | | | |
| K16180792 | bis(maltolato)oxo- | tyrosine phosphatase | MMP16 | matrix metalloproteinase-16 | −0.23044 | 1.26E−05 | 0.001414 | −0.27895 | M | 0.148517 | M | 0.090307 |
| | vanadium(IV) | inhibitor | | | | | | | | | | |
| K16180792 | bis(maltolato)oxo- | tyrosine phosphatase | MOXD1 | dbh-like monooxygenase | −0.22178 | 2.69E−05 | 0.00239 | −0.31384 | M | 0.148517 | M | 0.090307 |
| | vanadium(IV) | inhibitor | | protein 1 | | | | | | | | |
| K16180792 | bis(maltolato)oxo- | tyrosine phosphatase | MPDZ | multiple pdz domain protein | −0.20909 | 7.73E−05 | 0.004938 | −0.2474 | M | 0.148517 | M | 0.090307 |
| | vanadium(IV) | inhibitor | | | | | | | | | | |
| K16180792 | bis(maltolato)oxo- | tyrosine phosphatase | MYH10 | myosin-10 | −0.28316 | 6.49E−08 | 3.06E−05 | −0.30504 | M | 0.148517 | M | 0.090307 |
| | vanadium(IV) | inhibitor | | | | | | | | | | |
| K16180792 | bis(maltolato)oxo- | tyrosine phosphatase | MYLK | myosin light chain kinase; | −0.21442 | 4.99E−05 | 0.003669 | −0.27379 | M | 0.148517 | M | 0.090307 |
| | vanadium(IV) | inhibitor | | smooth muscle | | | | | | | | |
| K16180792 | bis(maltolato)oxo- | tyrosine phosphatase | NLGN1 | neuroligin-1 | −0.24202 | 4.37E−06 | 0.000659 | −0.29507 | M | 0.148517 | M | 0.090307 |
| | vanadium(IV) | inhibitor | | | | | | | | | | |
| K16180792 | bis(maltolato)oxo- | tyrosine phosphatase | NRROS | transforming growth factor | −0.18248 | 0.000581 | 0.018573 | −0.2668 | M | 0.148517 | M | 0.090307 |
| | vanadium(IV) | inhibitor | | beta activator lrrc33 | | | | | | | | |
| K16180792 | bis(maltolato)oxo- | tyrosine phosphatase | OBSL1 | obscurin-like protein 1 | −0.1858 | 0.000458 | 0.016001 | −0.25411 | M | 0.148517 | M | 0.090307 |
| | vanadium(IV) | inhibitor | | | | | | | | | | |
| K16180792 | bis(maltolato)oxo- | tyrosine phosphatase | PDE1C | calcium/calmodulin- | −0.20544 | 0.000104 | 0.006004 | −0.30418 | M | 0.148517 | M | 0.090307 |
| | vanadium(IV) | inhibitor | | dependent 3′; 5′-cyclic | | | | | | | | |
| | | | | nucleotide | | | | | | | | |
| | | | | phosphodiesterase 1c | | | | | | | | |
| K16180792 | bis(maltolato)oxo- | tyrosine phosphatase | PDE7B | camp-specific 3′; 5′-cyclic | −0.20025 | 0.000155 | 0.007898 | −0.24596 | M | 0.148517 | M | 0.090307 |
| | vanadium(IV) | inhibitor | | phosphodiesterase 7b | | | | | | | | |
| K16180792 | bis(maltolato)oxo- | tyrosine phosphatase | PIK3CD | phosphatidylinositol 4; 5- | −0.16595 | 0.001784 | 0.037181 | −0.33482 | M | 0.148517 | M | 0.090307 |
| | vanadium(IV) | inhibitor | | bisphosphate 3-kinase | | | | | | | | |
| | | | | catalytic subunit delta | | | | | | | | |
| | | | | isoform | | | | | | | | |
| K16180792 | bis(maltolato)oxo- | tyrosine phosphatase | PKNOX2 | homeobox protein pknox2 | −0.18428 | 0.000511 | 0.017132 | −0.25483 | M | 0.148517 | M | 0.090307 |
| | vanadium(IV) | inhibitor | | | | | | | | | | |
| K16180792 | bis(maltolato)oxo- | tyrosine phosphatase | PLP1 | myelin proteolipid protein | −0.28877 | 3.46E−08 | 1.9E−05 | −0.35348 | M | 0.148517 | M | 0.090307 |
| | vanadium(IV) | inhibitor | | | | | | | | | | |
| K16180792 | bis(maltolato)oxo- | tyrosine phosphatase | PRTG | protogenin | −0.33409 | 1.26E−10 | 2.89E−07 | −0.25364 | M | 0.148517 | M | 0.090307 |
| | vanadium(IV) | inhibitor | | | | | | | | | | |
| K16180792 | bis(maltolato)oxo- | tyrosine phosphatase | PTPRM | receptor-type tyrosine- | −0.30591 | 4.63E−09 | 4.35E−06 | −0.29048 | M | 0.148517 | M | 0.090307 |
| | vanadium(IV) | inhibitor | | protein phosphatase mu | | | | | | | | |
| K16180792 | bis(maltolato)oxo- | tyrosine phosphatase | QKI | protein quaking | −0.21829 | 3.61E−05 | 0.002939 | −0.24849 | M | 0.148517 | M | 0.090307 |
| | vanadium(IV) | inhibitor | | | | | | | | | | |
| K16180792 | bis(maltolato)oxo- | tyrosine phosphatase | RASSF4 | ras and rab interactor 2 | −0.20577 | 0.000101 | 0.005902 | −0.26113 | M | 0.148517 | M | 0.090307 |
| | vanadium(IV) | inhibitor | | | | | | | | | | |
| K16180792 | bis(maltolato)oxo- | tyrosine phosphatase | RASSF8 | ras association domain- | −0.25558 | 1.18E−06 | 0.000258 | −0.33255 | M | 0.148517 | M | 0.090307 |
| | vanadium(IV) | inhibitor | | containing protein 8 | | | | | | | | |
| K16180792 | bis(maltolato)oxo- | tyrosine phosphatase | RHOJ | rho-related gtp-binding | −0.26385 | 5.12E−07 | 0.000141 | −0.29192 | M | 0.148517 | M | 0.090307 |
| | vanadium(IV) | inhibitor | | protein rhoj | | | | | | | | |
| K16180792 | bis(maltolato)oxo- | tyrosine phosphatase | RUSC2 | iporin | −0.20522 | 0.000105 | 0.006078 | −0.25492 | M | 0.148517 | M | 0.090307 |
| | vanadium(IV) | inhibitor | | | | | | | | | | |
| K16180792 | bis(maltolato)oxo- | tyrosine phosphatase | SCARA5 | scavenger receptor class a | −0.17385 | 0.001056 | 0.027058 | −0.27249 | M | 0.148517 | M | 0.090307 |
| | vanadium(IV) | inhibitor | | member 5 | | | | | | | | |
| K16180792 | bis(maltolato)oxo- | tyrosine phosphatase | SHC4 | shc-transforming protein 4 | −0.2357 | 7.84E−06 | 0.001008 | −0.24436 | M | 0.148517 | M | 0.090307 |
| | vanadium(IV) | inhibitor | | | | | | | | | | |
| K16180792 | bis(maltolato)oxo- | tyrosine phosphatase | SHISA4 | protein shisa-4 | −0.25597 | 1.13E−06 | 0.000252 | −0.32166 | M | 0.148517 | M | 0.090307 |
| | vanadium(IV) | inhibitor | | | | | | | | | | |
| K16180792 | bis(maltolato)oxo- | tyrosine phosphatase | SHROOM4 | protein shroom4 | −0.29439 | 1.81E−08 | 1.2E−05 | −0.39439 | M | 0.148517 | M | 0.090307 |
| | vanadium(IV) | inhibitor | | | | | | | | | | |
| K16180792 | bis(maltolato)oxo- | tyrosine phosphatase | SLC35F1 | solute carrier family 35 | −0.17641 | 0.000887 | 0.024239 | −0.32088 | M | 0.148517 | M | 0.090307 |
| | vanadium(IV) | inhibitor | | member f1 | | | | | | | | |
| K16180792 | bis(maltolato)oxo- | tyrosine phosphatase | SLC6A12 | sodium- and chloride- | −0.23217 | 1.08E−05 | 0.001266 | −0.26619 | M | 0.148517 | M | 0.090307 |
| | vanadium(IV) | inhibitor | | dependent betaine | | | | | | | | |
| | | | | transporter | | | | | | | | |
| K16180792 | bis(maltolato)oxo- | tyrosine phosphatase | SNX10 | sorting nexin-10 | −0.27382 | 1.8E−07 | 6.54E−05 | −0.27325 | M | 0.148517 | M | 0.090307 |
| | vanadium(IV) | inhibitor | | | | | | | | | | |
| K16180792 | bis(maltolato)oxo- | tyrosine phosphatase | SORCS1 | vps10 domain-containing | −0.17359 | 0.001075 | 0.02736 | −0.27511 | M | 0.148517 | M | 0.090307 |
| | vanadium(IV) | inhibitor | | receptor sorcs1 | | | | | | | | |
| K16180792 | bis(maltolato)oxo- | tyrosine phosphatase | SPARC | sparc | −0.21202 | 6.09E−05 | 0.004199 | −0.31662 | M | 0.148517 | M | 0.090307 |
| | vanadium(IV) | inhibitor | | | | | | | | | | |
| K16180792 | bis(maltolato)oxo- | tyrosine phosphatase | SPART | spartin | −0.2644 | 4.83E−07 | 0.000135 | −0.35682 | M | 0.148517 | M | 0.090307 |
| | vanadium(IV) | inhibitor | | | | | | | | | | |
| K16180792 | bis(maltolato)oxo- | tyrosine phosphatase | SRGN | | −0.21734 | 3.92E−05 | 0.0031 | −0.25978 | M | 0.148517 | M | 0.090307 |
| | vanadium(IV) | inhibitor | | | | | | | | | | |
| K16180792 | bis(maltolato)oxo- | tyrosine phosphatase | SRPX | sushi repeat-containing | −0.27636 | 1.37E−07 | 5.34E−05 | −0.2889 | M | 0.148517 | M | 0.090307 |
| | vanadium(IV) | inhibitor | | protein srpx | | | | | | | | |
| K16180792 | bis(maltolato)oxo- | tyrosine phosphatase | STK32B | serine/threonine-protein | −0.25703 | 1.02E−06 | 0.000233 | −0.35902 | M | 0.148517 | M | 0.090307 |
| | vanadium(IV) | inhibitor | | kinase 32b | | | | | | | | |
| K16180792 | bis(maltolato)oxo- | tyrosine phosphatase | SYDE1 | rho gtpase-activating protein | −0.32736 | 3.09E−10 | 5.59E−07 | −0.29724 | M | 0.148517 | M | 0.090307 |
| | vanadium(IV) | inhibitor | | syde1 | | | | | | | | |
| K16180792 | bis(maltolato)oxo- | tyrosine phosphatase | TIMP2 | metalloproteinase inhibitor 2 | −0.21403 | 5.16E−05 | 0.003754 | −0.26124 | M | 0.148517 | M | 0.090307 |
| | vanadium(IV) | inhibitor | | | | | | | | | | |
| K16180792 | bis(maltolato)oxo- | tyrosine phosphatase | TRPV2 | transient receptor potential | −0.2301 | 1.3E−05 | 0.001444 | −0.24959 | M | 0.148517 | M | 0.090307 |
| | vanadium(IV) | inhibitor | | cation channel subfamily v | | | | | | | | |
| | | | | member 2 | | | | | | | | |
| K16180792 | bis(maltolato)oxo- | tyrosine phosphatase | WWTR1 | ww domain-containing | −0.29433 | 1.83E−08 | 1.21E−05 | −0.39193 | M | 0.148517 | M | 0.090307 |
| | vanadium(IV) | inhibitor | | transcription regulator | | | | | | | | |
| | | | | protein 1 | | | | | | | | |
| K19333160 | RKI-1447 | rho associated kinase | ACTR3B | actin-related protein 3b- | −0.16962 | 0.001124 | 0.028133 | −0.2398 | MSL | 0.046328 | MSL | 0.081223 |
| | | inhibitor | | related | | | | | | | | |
| K19333160 | RKI-1447 | rho associated kinase | ARL10 | adp-ribosylation factor-like | −0.16253 | 0.001812 | 0.03753 | −0.27817 | MSL | 0.046328 | MSL | 0.081223 |
| | | inhibitor | | protein 10 | | | | | | | | |
| K19333160 | RKI-1447 | rho associated kinase | CHST10 | carbohydrate | −0.16296 | 0.00176 | 0.036891 | −0.2705 | MSL | 0.046328 | MSL | 0.081223 |
| | | inhibitor | | sulfotransferase 10 | | | | | | | | |
| K19333160 | RKI-1447 | rho associated kinase | CNTNAP1 | contactin-associated protein | −0.27527 | 8.7E−08 | 3.81E−05 | −0.25274 | MSL | 0.046328 | MSL | 0.081223 |
| | | inhibitor | | 1 | | | | | | | | |
| K19333160 | RKI-1447 | rho associated kinase | DACT3 | dapper homolog 3 | −0.30877 | 1.59E−09 | 1.94E−06 | −0.27605 | MSL | 0.046328 | MSL | 0.081223 |
| | | inhibitor | | | | | | | | | | |
| K19333160 | RKI-1447 | rho associated kinase | DLX2 | homeobox protein dlx-2 | −0.16526 | 0.001511 | 0.033626 | −0.2927 | MSL | 0.046328 | MSL | 0.081223 |
| | | inhibitor | | | | | | | | | | |
| K19333160 | RKI-1447 | rho associated kinase | GPR162 | g-protein coupled receptor | −0.25479 | 7.83E−07 | 0.000191 | −0.28022 | MSL | 0.046328 | MSL | 0.081223 |
| | | inhibitor | | 162-related | | | | | | | | |
| K19333160 | RKI-1447 | rho associated kinase | GPSM1 | g-protein-signaling | −0.27133 | 1.35E−07 | 5.29E−05 | −0.24569 | MSL | 0.046328 | MSL | 0.081223 |
| | | inhibitor | | modulator 1 | | | | | | | | |
| K19333160 | RKI-1447 | rho associated kinase | HAND2 | heart- and neural crest | −0.25588 | 7E−07 | 0.000177 | −0.27974 | MSL | 0.046328 | MSL | 0.081223 |
| | | inhibitor | | derivatives-expressed | | | | | | | | |
| | | | | protein 2 | | | | | | | | |
| K19333160 | RKI-1447 | rho associated kinase | HGF | hepatocyte growth factor | −0.163 | 0.001756 | 0.036826 | −0.26698 | MSL | 0.046328 | MSL | 0.081223 |
| | | inhibitor | | | | | | | | | | |
| K19333160 | RKI-1447 | rho associated kinase | JAM3 | junctional adhesion | −0.24893 | 1.42E−06 | 0.000294 | −0.31976 | MSL | 0.046328 | MSL | 0.081223 |
| | | inhibitor | | molecule c | | | | | | | | |
| K19333160 | RKI-1447 | rho associated kinase | KBTBD6 | kelch repeat and btb | −0.16061 | 0.002056 | 0.04045 | −0.24298 | MSL | 0.046328 | MSL | 0.081223 |
| | | inhibitor | | domain-containing protein 6 | | | | | | | | |
| K19333160 | RKI-1447 | rho associated kinase | KIF5A | kinesin heavy chain isoform | −0.26001 | 4.55E−07 | 0.000129 | −0.2515 | MSL | 0.046328 | MSL | 0.081223 |
| | | inhibitor | | 5a | | | | | | | | |
| K19333160 | RKI-1447 | rho associated kinase | KRBA1 | protein krba1 | −0.19805 | 0.000137 | 0.007254 | −0.24991 | MSL | 0.046328 | MSL | 0.081223 |
| | | inhibitor | | | | | | | | | | |
| K19333160 | RKI-1447 | rho associated kinase | LIX1L | lix1-like protein | −0.23916 | 3.71E−06 | 0.000589 | −0.23907 | MSL | 0.046328 | MSL | 0.081223 |
| | | inhibitor | | | | | | | | | | |
| K19333160 | RKI-1447 | rho associated kinase | MICU3 | calcium uptake protein 3; | −0.21213 | 4.3E−05 | 0.003303 | −0.27166 | MSL | 0.046328 | MSL | 0.081223 |
| | | inhibitor | | mitochondrial | | | | | | | | |
| K19333160 | RKI-1447 | rho associated kinase | NLGN2 | neuroligin-2 | −0.24685 | 1.75E−06 | 0.000342 | −0.24008 | MSL | 0.046328 | MSL | 0.081223 |
| | | inhibitor | | | | | | | | | | |
| K19333160 | RKI-1447 | rho associated kinase | NUDT11 | diphosphoinositol | −0.2279 | 1.07E−05 | 0.001258 | −0.24921 | MSL | 0.046328 | MSL | 0.081223 |
| | | inhibitor | | polyphosphate | | | | | | | | |
| | | | | phosphohydrolase 3-beta | | | | | | | | |
| K19333160 | RKI-1447 | rho associated kinase | PIANP | pilr alpha-associated neural | −0.26366 | 3.09E−07 | 9.79E−05 | −0.24854 | MSL | 0.046328 | MSL | 0.081223 |
| | | inhibitor | | protein | | | | | | | | |
| K19333160 | RKI-1447 | rho associated kinase | PLEKHO1 | pleckstrin homology | −0.21951 | 2.27E−05 | 0.002127 | −0.2815 | MSL | 0.046328 | MSL | 0.081223 |
| | | inhibitor | | domain-containing family o | | | | | | | | |
| | | | | member 1 | | | | | | | | |
| K19333160 | RKI-1447 | rho associated kinase | PLPP7 | inactive phospholipid | −0.29234 | 1.21E−08 | 8.91E−06 | −0.31957 | MSL | 0.046328 | MSL | 0.081223 |
| | | inhibitor | | phosphatase 7 | | | | | | | | |
| K19333160 | RKI-1447 | rho associated kinase | RTL5 | retrotransposon gag-like | −0.19173 | 0.000224 | 0.010099 | −0.30955 | MSL | 0.046328 | MSL | 0.081223 |
| | | inhibitor | | protein 5 | | | | | | | | |
| K19333160 | RKI-1447 | rho associated kinase | SMO | smoothened homolog | −0.18192 | 0.000469 | 0.016249 | −0.2961 | MSL | 0.046328 | MSL | 0.081223 |
| | | inhibitor | | | | | | | | | | |
| K19333160 | RKI-1447 | rho associated kinase | STARD9 | star-related lipid transfer | −0.22352 | 1.59E−05 | 0.001665 | −0.30703 | MSL | 0.046328 | MSL | 0.081223 |
| | | inhibitor | | protein 9 | | | | | | | | |
| K19333160 | RKI-1447 | rho associated kinase | VASH1 | tubulinyl-tyr | −0.26064 | 4.26E−07 | 0.000123 | −0.25725 | MSL | 0.046328 | MSL | 0.081223 |
| | | inhibitor | | carboxypeptidase 1 | | | | | | | | |
| K19333160 | RKI-1447 | rho associated kinase | ZEB1 | zinc finger e-box-binding | −0.22431 | 1.48E−05 | 0.001586 | −0.24296 | MSL | 0.046328 | MSL | 0.081223 |
| | | inhibitor | | homeobox 1 | | | | | | | | |
| K19540840 | saracatinib | src inhibitor | DAPP1 | dual adapter for | −0.22658 | 1.14E−05 | 0.001314 | −0.23856 | IM | −0.18309 | IM | −0.10679 |
| | | | | phosphotyrosine and 3- | | | | | | | | |
| | | | | phosphotyrosine and 3- | | | | | | | | |
| | | | | phosphoinositide | | | | | | | | |
| K19540840 | saracatinib | src inhibitor | DENND1C | denn domain-containing | −0.21578 | 2.99E−05 | 0.002576 | −0.28936 | IM | −0.18309 | IM | −0.10679 |
| | | | | protein 1c | | | | | | | | |
| K19540840 | saracatinib | src inhibitor | DSG3 | desmoglein-3 | −0.235 | 5.19E−06 | 0.000746 | −0.25127 | IM | −0.18309 | IM | −0.10679 |
| K19540840 | saracatinib | src inhibitor | ITGB4 | integrin beta-4 | −0.3273 | 1.23E−10 | 2.84E−07 | −0.3346 | IM | −0.18309 | IM | −0.10679 |
| K19540840 | saracatinib | src inhibitor | KRT16 | keratin; type i cytoskeletal 16 | −0.22431 | 1.4E−05 | 0.001526 | −0.27834 | IM | −0.18309 | IM | −0.10679 |
| K19540840 | saracatinib | src inhibitor | KRT5 | keratin; type ii cytoskeletal 5 | −0.20934 | 5.18E−05 | 0.003767 | −0.29174 | IM | −0.18309 | IM | −0.10679 |
| K19540840 | saracatinib | src inhibitor | LTB | lymphotoxin-beta | −0.18179 | 0.000457 | 0.015979 | −0.27785 | IM | −0.18309 | IM | −0.10679 |
| K19540840 | saracatinib | src inhibitor | MAPK10 | mitogen-activated protein | −0.16933 | 0.001111 | 0.027933 | −0.27145 | IM | −0.18309 | IM | −0.10679 |
| | | | | kinase 10 | | | | | | | | |
| K19540840 | saracatinib | src inhibitor | PTAFR | platelet-activating factor | −0.21688 | 2.71E−05 | 0.002407 | −0.27523 | IM | −0.18309 | IM | −0.10679 |
| | | | | receptor | | | | | | | | |
| K19540840 | saracatinib | src inhibitor | PTPN6 | tyrosine-protein phosphatase | −0.27973 | 4.84E−08 | 2.48E−05 | −0.32068 | IM | −0.18309 | IM | −0.10679 |
| | | | | non-receptor type 6 | | | | | | | | |
| K22134346 | simvastatin | HMGCR inhibitor | ARHGAP31 | rho gtpase-activating protein 31 | −0.19459 | 0.00024 | 0.010527 | −0.30771 | M | 0.067288 | M | 0.0435 |
| K22134346 | simvastatin | HMGCR inhibitor | ARMCX1 | armadillo repeat-containing | −0.18048 | 0.000669 | 0.020287 | −0.29267 | M | 0.067288 | M | 0.0435 |
| | | | | x-linked protein 1 | | | | | | | | |
| K22134346 | simvastatin | HMGCR inhibitor | CNRIP1 | cb1 cannabinoid receptor- | −0.16703 | 0.001662 | 0.035614 | −0.24491 | M | 0.067288 | M | 0.0435 |
| | | | | interacting protein 1 | | | | | | | | |
| K22134346 | simvastatin | HMGCR inhibitor | CORO2B | coronin-2b | −0.1836 | 0.000536 | 0.01768 | −0.27781 | M | 0.067288 | M | 0.0435 |
| K22134346 | simvastatin | HMGCR inhibitor | CYBRD1 | cytochrome b reductase 1 | −0.17273 | 0.001139 | 0.028369 | −0.26682 | M | 0.067288 | M | 0.0435 |
| K22134346 | simvastatin | HMGCR inhibitor | EPHA3 | ephrin type-a receptor 3 | −0.21598 | 4.39E−05 | 0.003355 | −0.29345 | M | 0.067288 | M | 0.0435 |
| K22134346 | simvastatin | HMGCR inhibitor | EVC | ellis-van creveld syndrome | −0.19689 | 0.000201 | 0.009402 | −0.26818 | M | 0.067288 | M | 0.0435 |
| | | | | protein | | | | | | | | |
| K22134346 | simvastatin | HMGCR inhibitor | EVI2A | protein evi2a | −0.1743 | 0.001024 | 0.026557 | −0.28479 | M | 0.067288 | M | 0.0435 |
| K22134346 | simvastatin | HMGCR inhibitor | EXTL2 | exostosin-2-related | −0.18191 | 0.000605 | 0.019045 | −0.28106 | M | 0.067288 | M | 0.0435 |
| K22134346 | simvastatin | HMGCR inhibitor | FYN | tyrosine-protein kinase fyn | −0.1676 | 0.001602 | 0.034813 | −0.27314 | M | 0.067288 | M | 0.0435 |
| K22134346 | simvastatin | HMGCR inhibitor | IFFO1 | intermediate filament family | −0.15898 | 0.002779 | 0.048357 | −0.27534 | M | 0.067288 | M | 0.0435 |
| | | | | orphan 1 | | | | | | | | |
| K22134346 | simvastatin | HMGCR inhibitor | KANK2 | kn motif and ankyrin repeat | −0.22304 | 2.41E−05 | 0.002217 | −0.26204 | M | 0.067288 | M | 0.0435 |
| | | | | domain-containing protein 2 | | | | | | | | |
| K22134346 | simvastatin | HMGCR inhibitor | LPAR4 | lysophosphatidic acid | −0.22919 | 1.41E−05 | 0.001533 | −0.29553 | M | 0.067288 | M | 0.0435 |
| | | | | receptor 4 | | | | | | | | |
| K22134346 | simvastatin | HMGCR inhibitor | MOXD1 | dbh-like monooxygenase | −0.19563 | 0.000222 | 0.010019 | −0.3012 | M | 0.067288 | M | 0.0435 |
| | | | | protein 1 | | | | | | | | |
| K22134346 | simvastatin | HMGCR inhibitor | PRKD1 | | −0.20819 | 8.31E−05 | 0.005182 | −0.26241 | M | 0.067288 | M | 0.0435 |
| K22134346 | simvastatin | HMGCR inhibitor | RHOJ | rho-related gtp-binding | −0.19184 | 0.000294 | 0.012037 | −0.32304 | M | 0.067288 | M | 0.0435 |
| | | | | protein rhoj | | | | | | | | |
| K22134346 | simvastatin | HMGCR inhibitor | SLC35F1 | solute carrier family 35 | −0.17887 | 0.000748 | 0.021763 | −0.26588 | M | 0.067288 | M | 0.0435 |
| | | | | member f1 | | | | | | | | |
| K22134346 | simvastatin | HMGCR inhibitor | SRPX | sushi repeat-containing | −0.20612 | 9.81E−05 | 0.005797 | −0.27523 | M | 0.067288 | M | 0.0435 |
| | | | | protein srpx | | | | | | | | |
| K22134346 | simvastatin | HMGCR inhibitor | SYDE1 | rho gtpase-activating protein | −0.20277 | 0.000128 | 0.006929 | −0.29578 | M | 0.067288 | M | 0.0435 |
| | | | | syde1 | | | | | | | | |
| K22134346 | simvastatin | HMGCR inhibitor | ZCCHC24 | | −0.18723 | 0.000413 | 0.01496 | −0.28228 | M | 0.067288 | M | 0.0435 |
| K23190681 | AV-412 | protein tyrosine kinase | ADAM8 | disintegrin and | −0.19505 | 0.000188 | 0.008987 | −0.33616 | IM | −0.09469 | IM | −0.04654 |
| | | inhibitor | | metalloproteinase domain- | | | | | | | | |
| | | | | containing protein 8 | | | | | | | | |
| K23190681 | AV-412 | protein tyrosine kinase | ANKRD65 | ankyrin repeat domain- | −0.30013 | 5.7E−09 | 5.07E−06 | −0.38225 | IM | −0.09469 | IM | −0.04654 |
| | | inhibitor | | containing protein 65 | | | | | | | | |
| K23190681 | AV-412 | protein tyrosine kinase | ARHGDIB | rho gdp-dissociation | −0.24095 | 3.53E−06 | 0.000571 | −0.30378 | IM | −0.09469 | IM | −0.04654 |
| | | inhibitor | | inhibitor 2 | | | | | | | | |
| K23190681 | AV-412 | protein tyrosine kinase | ARHGEF4 | rho guanine nucleotide | −0.23562 | 5.86E−06 | 0.000814 | −0.28733 | IM | −0.09469 | IM | −0.04654 |
| | | inhibitor | | exchange factor 4 | | | | | | | | |
| K23190681 | AV-412 | protein tyrosine kinase | CLEC12A | c-type lectin domain family | −0.18846 | 0.000311 | 0.012492 | −0.26652 | IM | −0.09469 | IM | −0.04654 |
| | | inhibitor | | 12 member a | | | | | | | | |
| K23190681 | AV-412 | protein tyrosine kinase | COL17A1 | collagen alpha-1 | −0.36548 | 7E−13 | 6.5E−09 | −0.38565 | IM | −0.09469 | IM | −0.04654 |
| | | inhibitor | | | | | | | | | | |
| K23190681 | AV-412 | protein tyrosine kinase | CXCL16 | c-x-c motif chemokine 16 | −0.23921 | 4.17E−06 | 0.000637 | −0.27224 | IM | −0.09469 | IM | −0.04654 |
| | | inhibitor | | | | | | | | | | |
| K23190681 | AV-412 | protein tyrosine kinase | CYP4B1 | cytochrome p450 4b1 | −0.2049 | 8.61E−05 | 0.005306 | −0.29417 | IM | −0.09469 | IM | −0.04654 |
| | | inhibitor | | | | | | | | | | |
| K23190681 | AV-412 | protein tyrosine kinase | DAPP1 | dual adapter for | −0.42353 | 3.45E−17 | 5.17E−12 | −0.48092 | IM | −0.09469 | IM | −0.04654 |
| | | inhibitor | | phosphotyrosine and 3- | | | | | | | | |
| | | | | phosphotyrosine and 3- | | | | | | | | |
| | | | | phosphoinositide | | | | | | | | |
| K23190681 | AV-412 | protein tyrosine kinase | DENND1C | denn domain-containing | −0.30432 | 3.4E−09 | 3.42E−06 | −0.30076 | IM | −0.09469 | IM | −0.04654 |
| | | inhibitor | | protein 1c | | | | | | | | |
| K23190681 | AV-412 | protein tyrosine kinase | DSG3 | desmoglein-3 | −0.40941 | 4.61E−16 | 3.68E−11 | −0.35145 | IM | −0.09469 | IM | −0.04654 |
| | | inhibitor | | | | | | | | | | |
| K23190681 | AV-412 | protein tyrosine kinase | FGD3 | fyve; rhogef and ph domain- | −0.30954 | 1.77E−09 | 2.1E−06 | −0.38366 | IM | −0.09469 | IM | −0.04654 |
| | | inhibitor | | containing protein 3 | | | | | | | | |
| K23190681 | AV-412 | protein tyrosine kinase | HSH2D | hematopoietic sh2 domain- | −0.2356 | 5.87E−06 | 0.000815 | −0.32561 | IM | −0.09469 | IM | −0.04654 |
| | | inhibitor | | containing protein | | | | | | | | |
| K23190681 | AV-412 | protein tyrosine kinase | IL23A | interleukin-23 subunit alpha | −0.18689 | 0.00035 | 0.013472 | −0.24999 | IM | −0.09469 | IM | −0.04654 |
| | | inhibitor | | | | | | | | | | |
| K23190681 | AV-412 | protein tyrosine kinase | ITGB4 | integrin beta-4 | −0.30699 | 2.44E−09 | 2.7E−06 | −0.38114 | IM | −0.09469 | IM | −0.04654 |
| | | inhibitor | | | | | | | | | | |
| K23190681 | AV-412 | protein tyrosine kinase | KRT14 | keratin; type i cytoskeletal | −0.32808 | 1.57E−10 | 3.48E−07 | −0.28432 | IM | −0.09469 | IM | −0.04654 |
| | | inhibitor | | 14 | | | | | | | | |
| K23190681 | AV-412 | protein tyrosine kinase | KRT16 | keratin; type i cytoskeletal | −0.35228 | 5.13E−12 | 2.72E−08 | −0.36828 | IM | −0.09469 | IM | −0.04654 |
| | | inhibitor | | 16 | | | | | | | | |
| K23190681 | AV-412 | protein tyrosine kinase | KRT17 | keratin; type i cytoskeletal | −0.41511 | 1.64E−16 | 2.11E−11 | −0.41276 | IM | −0.09469 | IM | −0.04654 |
| | | inhibitor | | 17 | | | | | | | | |
| K23190681 | AV-412 | protein tyrosine kinase | KRT5 | keratin; type ii cytoskeletal | −0.40516 | 9.82E−16 | 5.2E−11 | −0.4264 | IM | −0.09469 | IM | −0.04654 |
| | | inhibitor | | 5 | | | | | | | | |
| K23190681 | AV-412 | protein tyrosine kinase | LGALS9 | galectin-9 | −0.21804 | 2.86E−05 | 0.002496 | −0.28354 | IM | −0.09469 | IM | −0.04654 |
| | | inhibitor | | | | | | | | | | |
| K23190681 | AV-412 | protein tyrosine kinase | LPAR5 | lysophosphatidic acid | −0.30804 | 2.14E−09 | 2.43E−06 | −0.28807 | IM | −0.09469 | IM | −0.04654 |
| | | inhibitor | | receptor 5 | | | | | | | | |
| K23190681 | AV-412 | protein tyrosine kinase | PTAFR | platelet-activating factor | −0.42507 | 2.58E−17 | 4.64E−12 | −0.49078 | IM | −0.09469 | IM | −0.04654 |
| | | inhibitor | | receptor | | | | | | | | |
| K23190681 | AV-412 | protein tyrosine kinase | PTK7 | inactive tyrosine-protein | −0.1563 | 0.002865 | 0.049234 | −0.25171 | IM | −0.09469 | IM | −0.04654 |
| | | inhibitor | | kinase 7 | | | | | | | | |
| K23190681 | AV-412 | protein tyrosine kinase | PTPN6 | tyrosine-protein phosphatase | −0.29966 | 6.03E−09 | 5.3E−06 | −0.36555 | IM | −0.09469 | IM | −0.04654 |
| | | inhibitor | | non-receptor type 6 | | | | | | | | |
| K23190681 | AV-412 | protein tyrosine kinase | RASSF5 | ras association domain- | −0.16831 | 0.001309 | 0.0308 | −0.24321 | IM | −0.09469 | IM | −0.04654 |
| | | inhibitor | | containing protein 5 | | | | | | | | |
| K23190681 | AV-412 | protein tyrosine kinase | S100A8 | protein s100-a8 | −0.25632 | 7.7E−07 | 0.000189 | −0.34257 | IM | −0.09469 | IM | −0.04654 |
| | | inhibitor | | | | | | | | | | |
| K23190681 | AV-412 | protein tyrosine kinase | SH3BP1 | bargin-related | −0.37018 | 3.36E−13 | 3.83E−09 | −0.36164 | IM | −0.09469 | IM | −0.04654 |
| | | inhibitor | | | | | | | | | | |
| K23190681 | AV-412 | protein tyrosine kinase | SIRPB2 | signal-regulatory protein | −0.19284 | 0.000223 | 0.010069 | −0.26026 | IM | −0.09469 | IM | −0.04654 |
| | | inhibitor | | beta-2 | | | | | | | | |
| K23190681 | AV-412 | protein tyrosine kinase | UNC13D | protein unc-13 homolog d | −0.17151 | 0.001052 | 0.026995 | −0.31853 | IM | −0.09469 | IM | −0.04654 |
| | | inhibitor | | | | | | | | | | |
| K23190681 | AV-412 | protein tyrosine kinase | VAV1 | proto-oncogene vav | −0.23139 | 8.68E−06 | 0.001085 | −0.24926 | IM | −0.09469 | IM | −0.04654 |
| | | inhibitor | | | | | | | | | | |
| K23190681 | AV-412 | protein tyrosine kinase | XCL1 | cytokine scm-1 beta-related | −0.22931 | 1.05E−05 | 0.001242 | −0.28227 | IM | −0.09469 | IM | −0.04654 |
| | | inhibitor | | | | | | | | | | |
| K23925186 | oridonin | BCL inhibitor | DACT3 | dapper homolog 3 | −0.19512 | 0.000191 | 0.009082 | −0.25452 | M | 0.132126 | M | 0.182341 |
| K23925186 | oridonin | BCL inhibitor | FAM78A | protein fam78a | −0.18993 | 0.000284 | 0.01177 | −0.29067 | M | 0.132126 | M | 0.182341 |
| K23925186 | oridonin | BCL inhibitor | FMNL3 | formin-like protein 3 | −0.18322 | 0.000467 | 0.016206 | −0.25822 | M | 0.132126 | M | 0.182341 |
| K23925186 | oridonin | BCL inhibitor | LZTS2 | zipper putative tumor | −0.17041 | 0.001153 | 0.028547 | −0.25743 | M | 0.132126 | M | 0.182341 |
| | | | | suppressor 2-related | | | | | | | | |
| K23925186 | oridonin | BCL inhibitor | MEX3A | rna-binding protein mex3a | −0.18672 | 0.000361 | 0.013741 | −0.24883 | M | 0.132126 | M | 0.182341 |
| K23925186 | oridonin | BCL inhibitor | MYH10 | myosin-10 | −0.19038 | 0.000275 | 0.011509 | −0.23849 | M | 0.132126 | M | 0.182341 |
| K23925186 | oridonin | BCL inhibitor | PHC1 | polyhomeotic-like protein 1 | −0.18315 | 0.00047 | 0.016262 | −0.25094 | M | 0.132126 | M | 0.182341 |
| K23925186 | oridonin | BCL inhibitor | PLPP7 | inactive phospholipid | −0.22048 | 2.37E−05 | 0.00219 | −0.26422 | M | 0.132126 | M | 0.182341 |
| | | | | phosphatase 7 | | | | | | | | |
| K26603252 | PD-153035 | EGFR inhibitor | ALOX5 | polyunsaturated fatty acid 5- | −0.25089 | 1.58E−06 | 0.000318 | −0.3717 | IM | −0.12324 | IM | −0.03709 |
| | | | | lipoxy genase | | | | | | | | |
| K26603252 | PD-153035 | EGFR inhibitor | ANKRD65 | ankyrin repeat domain- | −0.26685 | 3.1E−07 | 9.81E−05 | −0.32904 | IM | −0.12324 | IM | −0.03709 |
| | | | | containing protein 65 | | | | | | | | |
| K26603252 | PD-153035 | EGFR inhibitor | COL17A1 | collagen alpha-1 | −0.29128 | 2.07E−08 | 1.32E−05 | −0.41109 | IM | −0.12324 | IM | −0.03709 |
| K26603252 | PD-153035 | EGFR inhibitor | CXCL16 | c-x-c motif chemokine 16 | −0.25839 | 7.44E−07 | 0.000185 | −0.37765 | IM | −0.12324 | IM | −0.03709 |
| K26603252 | PD-153035 | EGFR inhibitor | CYP4B1 | cytochrome p450 4b1 | −0.16797 | 0.001446 | 0.032741 | −0.28801 | IM | −0.12324 | IM | −0.03709 |
| K26603252 | PD-153035 | EGFR inhibitor | DAPP1 | dual adapter for | −0.30983 | 2.21E−09 | 2.51E−06 | −0.42316 | IM | −0.12324 | IM | −0.03709 |
| | | | | phosphotyrosine and 3- | | | | | | | | |
| | | | | phosphotyrosine and 3- | | | | | | | | |
| | | | | phosphoinositide | | | | | | | | |
| K26603252 | PD-153035 | EGFR inhibitor | DENND1C | denn domain-containing | −0.31092 | 1.93E−09 | 2.23E−06 | −0.42894 | IM | −0.12324 | IM | −0.03709 |
| | | | | protein 1c | | | | | | | | |
| K26603252 | PD-153035 | EGFR inhibitor | DENND2D | denn domain-containing | −0.24765 | 2.17E−06 | 0.000399 | −0.30233 | IM | −0.12324 | IM | −0.03709 |
| | | | | protein 2d | | | | | | | | |
| K26603252 | PD-153035 | EGFR inhibitor | DSG3 | desmoglein-3 | −0.35668 | 3.77E−12 | 2.15E−08 | −0.34572 | IM | −0.12324 | IM | −0.03709 |
| K26603252 | PD-153035 | EGFR inhibitor | FGD3 | fyve; rhogef and ph domain- | −0.31466 | 1.21E−09 | 1.59E−06 | −0.40356 | IM | −0.12324 | IM | −0.03709 |
| | | | | containing protein 3 | | | | | | | | |
| K26603252 | PD-153035 | EGFR inhibitor | HSH2D | hematopoietic sh2 domain- | −0.22028 | 2.68E−05 | 0.002388 | −0.30664 | IM | −0.12324 | IM | −0.03709 |
| | | | | containing protein | | | | | | | | |
| K26603252 | PD-153035 | EGFR inhibitor | ITGB4 | integrin beta-4 | −0.34404 | 2.34E−11 | 8.31E−08 | −0.42238 | IM | −0.12324 | IM | −0.03709 |
| K26603252 | PD-153035 | EGFR inhibitor | KRT16 | keratin; type i cytoskeletal 16 | −0.35401 | 5.59E−12 | 2.92E−08 | −0.34579 | IM | −0.12324 | IM | −0.03709 |
| K26603252 | PD-153035 | EGFR inhibitor | KRT17 | keratin; type i cytoskeletal 17 | −0.30703 | 3.13E−09 | 3.24E−06 | −0.29342 | IM | −0.12324 | IM | −0.03709 |
| K26603252 | PD-153035 | EGFR inhibitor | KRT5 | keratin; type ii cytoskeletal 5 | −0.34628 | 1.71E−11 | 6.71E−08 | −0.35965 | IM | −0.12324 | IM | −0.03709 |
| K26603252 | PD-153035 | EGFR inhibitor | LPAR5 | lysophosphatidic acid | −0.36061 | 2.1E−12 | 1.44E−08 | −0.30522 | IM | −0.12324 | IM | −0.03709 |
| | | | | receptor 5 | | | | | | | | |
| K26603252 | PD-153035 | EGFR inhibitor | PTAFR | platelet-activating factor | −0.34036 | 3.93E−11 | 1.22E−07 | −0.46794 | IM | −0.12324 | IM | −0.03709 |
| | | | | receptor | | | | | | | | |
| K26603252 | PD-153035 | EGFR inhibitor | PTPN6 | tyrosine-protein phosphatase | −0.37993 | 1.05E−13 | 1.67E−09 | −0.44809 | IM | −0.12324 | IM | −0.03709 |
| | | | | non-receptor type 6 | | | | | | | | |
| K26603252 | PD-153035 | EGFR inhibitor | S100A8 | protein s100-a8 | −0.17309 | 0.001024 | 0.026552 | −0.32322 | IM | −0.12324 | IM | −0.03709 |
| K26603252 | PD-153035 | EGFR inhibitor | SH3BP1 | bargin-related | −0.27535 | 1.24E−07 | 4.99E−05 | −0.27597 | IM | −0.12324 | IM | −0.03709 |
| K26603252 | PD-153035 | EGFR inhibitor | SIRPB2 | signal-regulatory protein | −0.18599 | 0.000411 | 0.014909 | −0.26662 | IM | −0.12324 | IM | −0.03709 |
| | | | | beta-2 | | | | | | | | |
| K26603252 | PD-153035 | EGFR inhibitor | TNFSF10 | tumor necrosis factor ligand | −0.32407 | 3.57E−10 | 6.27E−07 | −0.24719 | IM | −0.12324 | IM | −0.03709 |
| | | | | superfamily member 10 | | | | | | | | |
| K26603252 | PD-153035 | EGFR inhibitor | UNC13D | protein unc-13 homolog d | −0.21 | 6.37E−05 | 0.004334 | −0.26712 | IM | −0.12324 | IM | −0.03709 |
| K26603252 | PD-153035 | EGFR inhibitor | VSIR | v-type immunoglobulin | −0.18997 | 0.000307 | 0.012364 | −0.24091 | IM | −0.12324 | IM | −0.03709 |
| | | | | domain-containing | | | | | | | | |
| | | | | suppressor of t-cell | | | | | | | | |
| | | | | activation | | | | | | | | |
| K26603252 | PD-153035 | EGFR inhibitor | ZMYND15 | zinc finger mynd domain- | −0.2211 | 2.5E−05 | 0.002273 | −0.25641 | IM | −0.12324 | IM | −0.03709 |
| | | | | containing protein 15 | | | | | | | | |
| K26818574 | BIX-01294 | histone lysine | BCL2A1 | bcl-2-related protein a1 | −0.18098 | 0.000503 | 0.016968 | −0.39482 | M | 0.083765 | M | 0.246345 |
| | | methyltransferase | | | | | | | | | | |
| | | inhibitor | | | | | | | | | | |
| K26818574 | BIX-01294 | histone lysine | CDH19 | cadherin-19 | −0.21375 | 3.74E−05 | 0.003006 | −0.30692 | M | 0.083765 | M | 0.246345 |
| | | methyltransferase | | | | | | | | | | |
| | | inhibitor | | | | | | | | | | |
| K26818574 | BIX-01294 | histone lysine | CHL1 | atp-dependent dna helicase | −0.27143 | 1.33E−07 | 5.25E−05 | −0.34136 | M | 0.083765 | M | 0.246345 |
| | | methyltransferase | | ddx11-related | | | | | | | | |
| | | inhibitor | | | | | | | | | | |
| K26818574 | BIX-01294 | histone lysine | COL19A1 | collagen alpha-1 | −0.19727 | 0.000146 | 0.007566 | −0.45596 | M | 0.083765 | M | 0.246345 |
| | | methyltransferase | | | | | | | | | | |
| | | inhibitor | | | | | | | | | | |
| K26818574 | BIX-01294 | histone lysine | DAAM2 | disheveled-associated | −0.19175 | 0.000224 | 0.010092 | −0.39135 | M | 0.083765 | M | 0.246345 |
| | | methyltransferase | | activator of morphogenesis | | | | | | | | |
| | | inhibitor | | 2 | | | | | | | | |
| K26818574 | BIX-01294 | histone lysine | EDNRB | endothelin receptor type b | −0.24594 | 1.91E−06 | 0.000366 | −0.4605 | M | 0.083765 | M | 0.246345 |
| | | methyltransferase | | | | | | | | | | |
| | | inhibitor | | | | | | | | | | |
| K26818574 | BIX-01294 | histone lysine | FAM180B | protein fam180b | −0.21795 | 2.6E−05 | 0.002336 | −0.48481 | M | 0.083765 | M | 0.246345 |
| | | methyltransferase | | | | | | | | | | |
| | | inhibitor | | | | | | | | | | |
| K26818574 | BIX-01294 | histone lysine | FCGR2A | low affinity | −0.17993 | 0.000543 | 0.017808 | −0.30448 | M | 0.083765 | M | 0.246345 |
| | | methyltransferase | | immunoglobulin gamma fc | | | | | | | | |
| | | inhibitor | | region receptor ii-a-related | | | | | | | | |
| K26818574 | BIX-01294 | histone lysine | FCGR2B | low affinity | −0.20657 | 6.85E−05 | 0.004551 | −0.2798 | M | 0.083765 | M | 0.246345 |
| | | methyltransferase | | immunoglobulin gamma fc | | | | | | | | |
| | | inhibitor | | region receptor ii-b | | | | | | | | |
| K26818574 | BIX-01294 | histone lysine | FCRLA | fc receptor-like a | −0.31111 | 1.18E−09 | 1.57E−06 | −0.4011 | M | 0.083765 | M | 0.246345 |
| | | methyltransferase | | | | | | | | | | |
| | | inhibitor | | | | | | | | | | |
| K26818574 | BIX-01294 | histone lysine | GAS7 | growth arrest-specific | −0.20989 | 5.19E−05 | 0.003772 | −0.42807 | M | 0.083765 | M | 0.246345 |
| | | methyltransferase | | protein 7 | | | | | | | | |
| | | inhibitor | | | | | | | | | | |
| K26818574 | BIX-01294 | histone lysine | GYPC | glycophorin-c | −0.18523 | 0.000367 | 0.013881 | −0.30433 | M | 0.083765 | M | 0.246345 |
| | | methyltransferase | | | | | | | | | | |
| | | inhibitor | | | | | | | | | | |
| K26818574 | BIX-01294 | histone lysine | IL12RB2 | interleukin-12 receptor | −0.18052 | 0.00052 | 0.017326 | −0.33563 | M | 0.083765 | M | 0.246345 |
| | | methyltransferase | | subunit beta-2 | | | | | | | | |
| | | inhibitor | | | | | | | | | | |
| K26818574 | BIX-01294 | histone lysine | IL16 | pro-interleukin-16 | −0.27842 | 6.1E−08 | 2.92E−05 | −0.4846 | M | 0.083765 | M | 0.246345 |
| | | methyltransferase | | | | | | | | | | |
| | | inhibitor | | | | | | | | | | |
| K26818574 | BIX-01294 | histone lysine | IRF4 | interferon regulatory factor | −0.2269 | 1.17E−05 | 0.00134 | −0.43689 | M | 0.083765 | M | 0.246345 |
| | | methyltransferase | | 4 | | | | | | | | |
| | | inhibitor | | | | | | | | | | |
| K26818574 | BIX-01294 | histone lysine | ITIH5 | inter-alpha-trypsin inhibitor | −0.17979 | 0.000548 | 0.017922 | −0.26924 | M | 0.083765 | M | 0.246345 |
| | | methyltransferase | | heavy chain h5 | | | | | | | | |
| | | inhibitor | | | | | | | | | | |
| K26818574 | BIX-01294 | histone lysine | KCNJ10 | atp-sensitive inward rectifier | −0.16171 | 0.001912 | 0.038705 | −0.27546 | M | 0.083765 | M | 0.246345 |
| | | methyltransferase | | potassium channel 10 | | | | | | | | |
| | | inhibitor | | | | | | | | | | |
| K26818574 | BIX-01294 | histone lysine | LPXN | leupaxin | −0.16953 | 0.001131 | 0.028245 | −0.25428 | M | 0.083765 | M | 0.246345 |
| | | methyltransferase | | | | | | | | | | |
| | | inhibitor | | | | | | | | | | |
| K26818574 | BIX-01294 | histone lysine | MIA | melanoma-derived growth | −0.20089 | 0.000109 | 0.006216 | −0.33434 | M | 0.083765 | M | 0.246345 |
| | | methyltransferase | | regulatory protein | | | | | | | | |
| | | inhibitor | | | | | | | | | | |
| K26818574 | BIX-01294 | histone lysine | NGFR | tumor necrosis factor | −0.15854 | 0.002351 | 0.043822 | −0.26243 | M | 0.083765 | M | 0.246345 |
| | | methyltransferase | | receptor superfamily | | | | | | | | |
| | | inhibitor | | member 16 | | | | | | | | |
| K26818574 | BIX-01294 | histone lysine | P2RX7 | p2x purinoceptor 7 | −0.21415 | 3.61E−05 | 0.002939 | −0.27448 | M | 0.083765 | M | 0.246345 |
| | | methyltransferase | | | | | | | | | | |
| | | inhibitor | | | | | | | | | | |
| K26818574 | BIX-01294 | histone lysine | PDZRN3 | e3 ubiquitin-protein ligase | −0.18642 | 0.000336 | 0.013121 | −0.2683 | M | 0.083765 | M | 0.246345 |
| | | methyltransferase | | pdzrn3 | | | | | | | | |
| | | inhibitor | | | | | | | | | | |
| K26818574 | BIX-01294 | histone lysine | PKNOX2 | homeobox protein pknox2 | −0.20258 | 9.5E−05 | 0.005672 | −0.35864 | M | 0.083765 | M | 0.246345 |
| | | methyltransferase | | | | | | | | | | |
| | | inhibitor | | | | | | | | | | |
| K26818574 | BIX-01294 | histone lysine | PLEKHO2 | pleckstrin homology | −0.1935 | 0.000196 | 0.009229 | −0.3448 | M | 0.083765 | M | 0.246345 |
| | | methyltransferase | | domain-containing family o | | | | | | | | |
| | | inhibitor | | member 2 | | | | | | | | |
| K26818574 | BIX-01294 | histone lysine | PLP1 | myelin proteolipid protein | −0.265 | 2.68E−07 | 8.82E−05 | −0.4139 | M | 0.083765 | M | 0.246345 |
| | | methyltransferase | | | | | | | | | | |
| | | inhibitor | | | | | | | | | | |
| K26818574 | BIX-01294 | histone lysine | PMP2 | myelin p2 protein | −0.30337 | 3.14E−09 | 3.24E−06 | −0.35054 | M | 0.083765 | M | 0.246345 |
| | | methyltransferase | | | | | | | | | | |
| | | inhibitor | | | | | | | | | | |
| K26818574 | BIX-01294 | histone lysine | PTCRA | pre t-cell antigen receptor | −0.24819 | 1.53E−06 | 0.000311 | −0.48186 | M | 0.083765 | M | 0.246345 |
| | | methyltransferase | | alpha | | | | | | | | |
| | | inhibitor | | | | | | | | | | |
| K26818574 | BIX-01294 | histone lysine | RENBP | n-acylglucosamine 2- | −0.1905 | 0.000247 | 0.010735 | −0.43127 | M | 0.083765 | M | 0.246345 |
| | | methyltransferase | | epimerase | | | | | | | | |
| | | inhibitor | | | | | | | | | | |
| K26818574 | BIX-01294 | histone lysine | RHOJ | rho-related gtp-binding | −0.22446 | 1.46E−05 | 0.00157 | −0.34455 | M | 0.083765 | M | 0.246345 |
| | | methyltransferase | | protein rhoj | | | | | | | | |
| | | inhibitor | | | | | | | | | | |
| K26818574 | BIX-01294 | histone lysine | SCML4 | sex comb on midleg-like | −0.1827 | 0.000443 | 0.015658 | −0.29749 | M | 0.083765 | M | 0.246345 |
| | | methyltransferase | | protein 4 | | | | | | | | |
| | | inhibitor | | | | | | | | | | |
| K26818574 | BIX-01294 | histone lysine | SGCD | delta-sarcoglycan | −0.19606 | 0.00016 | 0.008056 | −0.39099 | M | 0.083765 | M | 0.246345 |
| | | methyltransferase | | | | | | | | | | |
| | | inhibitor | | | | | | | | | | |
| K26818574 | BIX-01294 | histone lysine | SHC4 | shc-transforming protein 4 | −0.21965 | 2.24E−05 | 0.002108 | −0.34777 | M | 0.083765 | M | 0.246345 |
| | | methyltransferase | | | | | | | | | | |
| | | inhibitor | | | | | | | | | | |
| K26818574 | BIX-01294 | histone lysine | SORBS1 | sorbin and sh3 domain- | −0.15877 | 0.002316 | 0.043413 | −0.36063 | M | 0.083765 | M | 0.246345 |
| | | methyltransferase | | containing protein 1 | | | | | | | | |
| | | inhibitor | | | | | | | | | | |
| K26818574 | BIX-01294 | histone lysine | SORCS1 | vps10 domain-containing | −0.22926 | 9.41E−06 | 0.001151 | −0.30163 | M | 0.083765 | M | 0.246345 |
| | | methyltransferase | | receptor sorcs1 | | | | | | | | |
| | | inhibitor | | | | | | | | | | |
| K26818574 | BIX-01294 | histone lysine | ST8SIA1 | alpha-n-acetylneuraminide | −0.19011 | 0.000254 | 0.010944 | −0.27717 | M | 0.083765 | M | 0.246345 |
| | | methyltransferase | | alpha-2; 8-sialyltransferase | | | | | | | | |
| | | inhibitor | | | | | | | | | | |
| K26818574 | BIX-01294 | histone lysine | TAMALIN | protein tamalin | −0.20005 | 0.000117 | 0.006516 | −0.37086 | M | 0.083765 | M | 0.246345 |
| | | methyltransferase | | | | | | | | | | |
| | | inhibitor | | | | | | | | | | |
| K26818574 | BIX-01294 | histone lysine | TMEM229B | transmembrane protein 229b | −0.17272 | 0.000907 | 0.024562 | −0.29055 | M | 0.083765 | M | 0.246345 |
| | | methyltransferase | | | | | | | | | | |
| | | inhibitor | | | | | | | | | | |
| K26818574 | BIX-01294 | histone lysine | TRPV2 | transient receptor potential | −0.1858 | 0.000352 | 0.013517 | −0.41205 | M | 0.083765 | M | 0.246345 |
| | | methyltransferase | | cation channel subfamily v | | | | | | | | |
| | | inhibitor | | member 2 | | | | | | | | |
| K26818574 | BIX-01294 | histone lysine | WFDC1 | wap four-disulfide core | −0.22462 | 1.44E−05 | 0.001556 | −0.35978 | M | 0.083765 | M | 0.246345 |
| | | methyltransferase | | domain protein 1 | | | | | | | | |
| | | inhibitor | | | | | | | | | | |
| K28061410 | beta-lapachone | topoisomerase inhibitor | APOLD1 | apolipoprotein 1 domain- | −0.2156 | 4.53E−05 | 0.00343 | −0.24277 | M | 0.121653 | M | 0.182082 |
| | | | | containing protein 1 | | | | | | | | |
| K28061410 | beta-lapachone | topoisomerase inhibitor | DAAM2 | disheveled-associated | −0.23539 | 8.06E−06 | 0.001029 | −0.24866 | M | 0.121653 | M | 0.182082 |
| | | | | activator of morphogenesis 2 | | | | | | | | |
| K28061410 | beta-lapachone | topoisomerase inhibitor | FAM180B | protein fam180b | −0.26199 | 6.19E−07 | 0.000162 | −0.27998 | M | 0.121653 | M | 0.182082 |
| K28061410 | beta-lapachone | topoisomerase inhibitor | GAS7 | growth arrest-specific | −0.26458 | 4.75E−07 | 0.000133 | −0.31098 | M | 0.121653 | M | 0.182082 |
| | | | | protein 7 | | | | | | | | |
| K28061410 | beta-lapachone | topoisomerase inhibitor | IL12RB2 | interleukin-12 receptor | −0.20939 | 7.54E−05 | 0.00486 | −0.2616 | M | 0.121653 | M | 0.182082 |
| | | | | subunit beta-2 | | | | | | | | |
| K28061410 | beta-lapachone | topoisomerase inhibitor | IL 16 | pro-interleukin-16 | −0.261 | 6.85E−07 | 0.000174 | −0.26434 | M | 0.121653 | M | 0.182082 |
| K28061410 | beta-lapachone | topoisomerase inhibitor | IRF4 | interferon regulatory factor 4 | −0.27181 | 2.23E−07 | 7.65E−05 | −0.26084 | M | 0.121653 | M | 0.182082 |
| K28061410 | beta-lapachone | topoisomerase inhibitor | ITK | tyrosine-protein kinase | −0.18526 | 0.000476 | 0.016405 | −0.24098 | M | 0.121653 | M | 0.182082 |
| | | | | itk/tsk | | | | | | | | |
| K28061410 | beta-lapachone | topoisomerase inhibitor | LZTS1 | | −0.25159 | 1.75E−06 | 0.000342 | −0.26685 | M | 0.121653 | M | 0.182082 |
| K28061410 | beta-lapachone | topoisomerase inhibitor | PKNOX2 | homeobox protein pknox2 | −0.23233 | 1.06E−05 | 0.001255 | −0.28919 | M | 0.121653 | M | 0.182082 |
| K28061410 | beta-lapachone | topoisomerase inhibitor | PTCRA | pre t-cell antigen receptor | −0.22947 | 1.37E−05 | 0.001505 | −0.29461 | M | 0.121653 | M | 0.182082 |
| | | | | alpha | | | | | | | | |
| K28061410 | beta-lapachone | topoisomerase inhibitor | SHC4 | shc-transforming protein 4 | −0.18615 | 0.000447 | 0.015745 | −0.24831 | M | 0.121653 | M | 0.182082 |
| K28061410 | beta-lapachone | topoisomerase inhibitor | ST6GALNAC3 | alpha-n- | −0.21788 | 3.74E−05 | 0.003009 | −0.25187 | M | 0.121653 | M | 0.182082 |
| | | | | acetylgalactosaminide | | | | | | | | |
| | | | | alpha-2; 6-sialyltransferase 3 | | | | | | | | |
| K28061410 | beta-lapachone | topoisomerase inhibitor | TAMALIN | protein tamalin | −0.17178 | 0.001214 | 0.029433 | −0.26574 | M | 0.121653 | M | 0.182082 |
| K28824103 | genipin | choleretic agent | ABI2 | abl interactor 2 | −0.17755 | 0.000942 | 0.025176 | −0.25554 | M | 0.094785 | M | 0.105229 |
| K28824103 | genipin | choleretic agent | ADAM11 | disintegrin and | −0.18554 | 0.000542 | 0.017803 | −0.27342 | M | 0.094785 | M | 0.105229 |
| | | | | metalloproteinase domain- | | | | | | | | |
| | | | | containing protein 11 | | | | | | | | |
| K28824103 | genipin | choleretic agent | ADGRB2 | adhesion g protein-coupled | −0.18503 | 0.000562 | 0.018194 | −0.27272 | M | 0.094785 | M | 0.105229 |
| | | | | receptor b2 | | | | | | | | |
| K28824103 | genipin | choleretic agent | CAND2 | cullin-associated nedd8- | −0.20781 | 0.000103 | 0.005996 | −0.27123 | M | 0.094785 | M | 0.105229 |
| | | | | dissociated protein 2 | | | | | | | | |
| K28824103 | genipin | choleretic agent | CBX1 | chromobox protein homolog 1 | −0.25264 | 2.08E−06 | 0.000387 | −0.25015 | M | 0.094785 | M | 0.105229 |
| K28824103 | genipin | choleretic agent | CHD3 | chromodomain-helicase- | −0.17169 | 0.001391 | 0.031984 | −0.24417 | M | 0.094785 | M | 0.105229 |
| | | | | dna-binding protein 3 | | | | | | | | |
| K28824103 | genipin | choleretic agent | FAM171A2 | protein fam171a2 | −0.22398 | 2.76E−05 | 0.002434 | −0.25384 | M | 0.094785 | M | 0.105229 |
| K28824103 | genipin | choleretic agent | FOXO3B | forkhead box protein o3b- | −0.19201 | 0.000341 | 0.013251 | −0.27763 | M | 0.094785 | M | 0.105229 |
| | | | | related | | | | | | | | |
| K28824103 | genipin | choleretic agent | MPP2 | forkhead box protein m1 | −0.18453 | 0.000582 | 0.018604 | −0.32854 | M | 0.094785 | M | 0.105229 |
| K28824103 | genipin | choleretic agent | MRC2 | c-type mannose receptor 2 | −0.21017 | 8.57E−05 | 0.005291 | −0.26049 | M | 0.094785 | M | 0.105229 |
| K28824103 | genipin | choleretic agent | NLGN2 | neuroligin-2 | −0.21373 | 6.44E−05 | 0.004368 | −0.25824 | M | 0.094785 | M | 0.105229 |
| K28824103 | genipin | choleretic agent | PALM | paralemmin-1 | −0.17135 | 0.001422 | 0.032415 | −0.24524 | M | 0.094785 | M | 0.105229 |
| K28824103 | genipin | choleretic agent | PDZD4 | pdz domain-containing | −0.23529 | 1.03E−05 | 0.001227 | −0.24402 | M | 0.094785 | M | 0.105229 |
| | | | | protein 4 | | | | | | | | |
| K28824103 | genipin | choleretic agent | RBPMS2 | rna-binding protein with | −0.22239 | 3.16E−05 | 0.002676 | −0.3638 | M | 0.094785 | M | 0.105229 |
| | | | | multiple splicing 2 | | | | | | | | |
| K28824103 | genipin | choleretic agent | SMO | smoothened homolog | −0.21454 | 6.04E−05 | 0.004173 | −0.27256 | M | 0.094785 | M | 0.105229 |
| K28824103 | genipin | choleretic agent | TUB | tubby protein homolog | −0.22462 | 2.61E−05 | 0.002346 | −0.25911 | M | 0.094785 | M | 0.105229 |
| K28824103 | genipin | choleretic agent | USP22 | ubiquitin carboxyl-terminal | −0.17164 | 0.001396 | 0.032059 | −0.25991 | M | 0.094785 | M | 0.105229 |
| | | | | hydrolase 22 | | | | | | | | |
| K28824103 | genipin | choleretic agent | VASH1 | tubulinyl-tyr | −0.26042 | 9.71E−07 | 0.000225 | −0.28016 | M | 0.094785 | M | 0.105229 |
| | | | | carboxypeptidase 1 | | | | | | | | |
| K30159788 | RSV604 | RSV replication | FGFR1 | fibroblast growth factor | −0.16628 | 0.001799 | 0.037375 | −0.26638 | MSL | −0.0004 | MSL | 0.050206 |
| | | inhibitor | | receptor 1 | | | | | | | | |
| K30933884 | UNBS-5162 | CC chemokine receptor | ABI2 | abl interactor 2 | −0.1664 | 0.001399 | 0.032097 | −0.28321 | M | 0.098439 | M | 0.091475 |
| | | antagonist | | | | | | | | | | |
| K30933884 | UNBS-5162 | CC chemokine receptor | ADAM11 | disintegrin and | −0.19943 | 0.000123 | 0.006736 | −0.27114 | M | 0.098439 | M | 0.091475 |
| | | antagonist | | metalloproteinase domain- | | | | | | | | |
| | | | | containing protein 11 | | | | | | | | |
| K30933884 | UNBS-5162 | CC chemokine receptor | DLX6 | homeobox protein dlx-6 | −0.19115 | 0.000235 | 0.010384 | −0.25188 | M | 0.098439 | M | 0.091475 |
| | | antagonist | | | | | | | | | | |
| K30933884 | UNBS-5162 | CC chemokine receptor | EMILIN3 | emilin-3 | −0.18183 | 0.000473 | 0.016326 | −0.23795 | M | 0.098439 | M | 0.091475 |
| | | antagonist | | | | | | | | | | |
| K30933884 | UNBS-5162 | CC chemokine receptor | FAM171A2 | protein fam171a2 | −0.27943 | 5.44E−08 | 2.69E−05 | −0.27281 | M | 0.098439 | M | 0.091475 |
| | | antagonist | | | | | | | | | | |
| K30933884 | UNBS-5162 | CC chemokine receptor | FOXO3B | forkhead box protein o3b- | −0.2549 | 7.74E−07 | 0.00019 | −0.25677 | M | 0.098439 | M | 0.091475 |
| | | antagonist | | related | | | | | | | | |
| K30933884 | UNBS-5162 | CC chemokine receptor | GALNT17 | polypeptide n- | −0.21945 | 2.28E−05 | 0.002135 | −0.3209 | M | 0.098439 | M | 0.091475 |
| | | antagonist | | acetylgalactosaminyltransfer | | | | | | | | |
| | | | | ase-like 6 | | | | | | | | |
| K30933884 | UNBS-5162 | CC chemokine receptor | KCNJ4 | inward rectifier potassium | −0.19046 | 0.000248 | 0.01076 | −0.23833 | M | 0.098439 | M | 0.091475 |
| | | antagonist | | channel 4 | | | | | | | | |
| K30933884 | UNBS-5162 | CC chemokine receptor | MEX3A | rna-binding protein mex3a | −0.29311 | 1.1E−08 | 8.3E−06 | −0.24131 | M | 0.098439 | M | 0.091475 |
| | | antagonist | | | | | | | | | | |
| K30933884 | UNBS-5162 | CC chemokine receptor | MPP2 | forkhead box protein m1 | −0.1761 | 0.000715 | 0.021151 | −0.28776 | M | 0.098439 | M | 0.091475 |
| | | antagonist | | | | | | | | | | |
| K30933884 | UNBS-5162 | CC chemokine receptor | MSI1 | rna-binding protein musashi | −0.21573 | 3.15E−05 | 0.002674 | −0.34931 | M | 0.098439 | M | 0.091475 |
| | | antagonist | | homolog 1 | | | | | | | | |
| K30933884 | UNBS-5162 | CC chemokine receptor | NEXMIF | neurite extension and | −0.15635 | 0.002705 | 0.047585 | −0.29824 | M | 0.098439 | M | 0.091475 |
| | | antagonist | | migration factor | | | | | | | | |
| K30933884 | UNBS-5162 | CC chemokine receptor | POLR2A | dna-directed rna polymerase | −0.16784 | 0.001269 | 0.03025 | −0.2576 | M | 0.098439 | M | 0.091475 |
| | | antagonist | | ii subunit rpb1 | | | | | | | | |
| K30933884 | UNBS-5162 | CC chemokine receptor | RBPMS2 | rna-binding protein with | −0.19022 | 0.000252 | 0.010882 | −0.25586 | M | 0.098439 | M | 0.091475 |
| | | antagonist | | multiple splicing 2 | | | | | | | | |
| K30933884 | UNBS-5162 | CC chemokine receptor | RCOR2 | rest corepressor 2 | −0.23613 | 4.96E−06 | 0.000722 | −0.25441 | M | 0.098439 | M | 0.091475 |
| | | antagonist | | | | | | | | | | |
| K30933884 | UNBS-5162 | CC chemokine receptor | SALL2 | sal-like protein 2 | −0.21383 | 3.71E−05 | 0.002992 | −0.30324 | M | 0.098439 | M | 0.091475 |
| | | antagonist | | | | | | | | | | |
| K30933884 | UNBS-5162 | CC chemokine receptor | SMO | smoothened homolog | −0.21816 | 2.55E−05 | 0.002307 | −0.25619 | M | 0.098439 | M | 0.091475 |
| | | antagonist | | | | | | | | | | |
| K30933884 | UNBS-5162 | CC chemokine receptor | TMPO | lamina-associated | −0.19738 | 0.000144 | 0.00752 | −0.25031 | M | 0.098439 | M | 0.091475 |
| | | antagonist | | polypeptide 2; isoform alpha | | | | | | | | |
| K30933884 | UNBS-5162 | CC chemokine receptor | ZC3H12B | ribonuclease zc3h12b- | −0.16416 | 0.001625 | 0.035123 | −0.27218 | M | 0.098439 | M | 0.091475 |
| | | antagonist | | related | | | | | | | | |
| K31698212 | icotinib | EGFR inhibitor | APOL1 | apolipoprotein 11 | −0.18094 | 0.000487 | 0.016629 | −0.24361 | IM | −0.13826 | IM | −0.10753 |
| K31698212 | icotinib | EGFR inhibitor | DAPP1 | dual adapter for | −0.17078 | 0.001005 | 0.026235 | −0.31509 | IM | −0.13826 | IM | −0.10753 |
| | | | | phosphotyrosine and 3- | | | | | | | | |
| | | | | phosphotyrosine and 3- | | | | | | | | |
| | | | | phosphoinositide | | | | | | | | |
| K31698212 | icotinib | EGFR inhibitor | ITGB4 | integrin beta-4 | −0.21779 | 2.5E−05 | 0.002277 | −0.31123 | IM | −0.13826 | IM | −0.10753 |
| K31698212 | icotinib | EGFR inhibitor | KRT16 | keratin; type i cytoskeletal 16 | −0.19422 | 0.000178 | 0.008644 | −0.24565 | IM | −0.13826 | IM | −0.10753 |
| K31698212 | icotinib | EGFR inhibitor | NMI | n-myc-interactor | −0.22911 | 9.03E−06 | 0.001118 | −0.27643 | IM | −0.13826 | IM | −0.10753 |
| K31698212 | icotinib | EGFR inhibitor | NTN4 | netrin-4 | −0.16545 | 0.001447 | 0.032749 | −0.24283 | IM | −0.13826 | IM | −0.10753 |
| K31698212 | icotinib | EGFR inhibitor | PTAFR | platelet-activating factor | −0.18871 | 0.000272 | 0.011433 | −0.3502 | IM | −0.13826 | IM | −0.10753 |
| | | | | receptor | | | | | | | | |
| K31698212 | icotinib | EGFR inhibitor | PTPN6 | tyrosine-protein phosphatase | −0.18482 | 0.000365 | 0.013828 | −0.28189 | IM | −0.13826 | IM | −0.10753 |
| | | | | non-receptor type 6 | | | | | | | | |
| K31698212 | icotinib | EGFR inhibitor | VSIR | v-type immunoglobulin | −0.16724 | 0.001282 | 0.030424 | −0.27989 | IM | −0.13826 | IM | −0.10753 |
| | | | | domain-containing | | | | | | | | |
| | | | | suppressor of t-cell | | | | | | | | |
| | | | | activation | | | | | | | | |
| K31866293 | TAK-632 | RAF inhibitor | ASB2 | ankyrin repeat and socs box | −0.22412 | 1.5E−05 | 0.001605 | −0.27406 | M | 0.084888 | M | 0.20887 |
| | | | | protein 2 | | | | | | | | |
| K31866293 | TAK-632 | RAF inhibitor | BCL2A1 | bcl-2-related protein al | −0.23617 | 4.93E−06 | 0.00072 | −0.26144 | M | 0.084888 | M | 0.20887 |
| K31866293 | TAK-632 | RAF inhibitor | C3orf70 | upf0524 protein c3orf70 | −0.17213 | 0.000945 | 0.025219 | −0.34983 | M | 0.084888 | M | 0.20887 |
| K31866293 | TAK-632 | RAF inhibitor | CDH19 | cadherin-19 | −0.25999 | 4.56E−07 | 0.000129 | −0.28578 | M | 0.084888 | M | 0.20887 |
| K31866293 | TAK-632 | RAF inhibitor | COL19A1 | collagen alpha-1 | −0.24146 | 2.97E−06 | 0.000501 | −0.37648 | M | 0.084888 | M | 0.20887 |
| K31866293 | TAK-632 | RAF inhibitor | COL9A3 | collagen alpha-3 | −0.20202 | 9.95E−05 | 0.005851 | −0.27006 | M | 0.084888 | M | 0.20887 |
| K31866293 | TAK-632 | RAF inhibitor | CSPG4 | chondroitin sulfate | −0.25042 | 1.22E−06 | 0.000265 | −0.31554 | M | 0.084888 | M | 0.20887 |
| | | | | proteoglycan 4 | | | | | | | | |
| K31866293 | TAK-632 | RAF inhibitor | CTLA4 | cytotoxic t-lymphocyte | −0.18718 | 0.000318 | 0.012649 | −0.24656 | M | 0.084888 | M | 0.20887 |
| | | | | protein 4 | | | | | | | | |
| K31866293 | TAK-632 | RAF inhibitor | CYGB | cytoglobin | −0.21969 | 2.23E−05 | 0.002102 | −0.24697 | M | 0.084888 | M | 0.20887 |
| K31866293 | TAK-632 | RAF inhibitor | DAAM2 | disheveled-associated | −0.17349 | 0.000859 | 0.023734 | −0.34372 | M | 0.084888 | M | 0.20887 |
| | | | | activator of morphogenesis 2 | | | | | | | | |
| K31866293 | TAK-632 | RAF inhibitor | EDNRB | endothelin receptor type b | −0.27431 | 9.68E−08 | 4.12E−05 | −0.30546 | M | 0.084888 | M | 0.20887 |
| K31866293 | TAK-632 | RAF inhibitor | FAM180B | protein fam180b | −0.19812 | 0.000136 | 0.007225 | −0.30667 | M | 0.084888 | M | 0.20887 |
| K31866293 | TAK-632 | RAF inhibitor | FCGR2A | low affinity | −0.20097 | 0.000108 | 0.006187 | −0.27229 | M | 0.084888 | M | 0.20887 |
| | | | | immunoglobulin gamma fc | | | | | | | | |
| | | | | region receptor ii-a-related | | | | | | | | |
| K31866293 | TAK-632 | RAF inhibitor | FCGR2B | low affinity | −0.25496 | 7.7E−07 | 0.000189 | −0.28451 | M | 0.084888 | M | 0.20887 |
| | | | | immunoglobulin gamma fc | | | | | | | | |
| | | | | region receptor ii-b | | | | | | | | |
| K31866293 | TAK-632 | RAF inhibitor | FCRLA | fc receptor-like a | −0.25098 | 1.15E−06 | 0.000255 | −0.36943 | M | 0.084888 | M | 0.20887 |
| K31866293 | TAK-632 | RAF inhibitor | GAS7 | growth arrest-specific | −0.22745 | 1.11E−05 | 0.001292 | −0.37157 | M | 0.084888 | M | 0.20887 |
| | | | | protein 7 | | | | | | | | |
| K31866293 | TAK-632 | RAF inhibitor | GNG2 | guanine nucleotide-binding | −0.23746 | 4.37E−06 | 0.000659 | −0.25302 | M | 0.084888 | M | 0.20887 |
| | | | | protein g | | | | | | | | |
| K31866293 | TAK-632 | RAF inhibitor | GPR55 | g-protein coupled receptor 55 | −0.18214 | 0.000462 | 0.016073 | −0.2411 | M | 0.084888 | M | 0.20887 |
| K31866293 | TAK-632 | RAF inhibitor | GSN | gelsolin | −0.16453 | 0.001586 | 0.034612 | −0.28152 | M | 0.084888 | M | 0.20887 |
| K31866293 | TAK-632 | RAF inhibitor | IL12RB2 | interleukin-12 receptor | −0.23792 | 4.18E−06 | 0.000637 | −0.24854 | M | 0.084888 | M | 0.20887 |
| | | | | subunit beta-2 | | | | | | | | |
| K31866293 | TAK-632 | RAF inhibitor | IL16 | pro-interleukin-16 | −0.2221 | 1.8E−05 | 0.001814 | −0.40428 | M | 0.084888 | M | 0.20887 |
| K31866293 | TAK-632 | RAF inhibitor | IRF4 | interferon regulatory factor 4 | −0.25383 | 8.64E−07 | 0.000206 | −0.3306 | M | 0.084888 | M | 0.20887 |
| K31866293 | TAK-632 | RAF inhibitor | ITGA9 | integrin alpha-9 | −0.20334 | 8.94E−05 | 0.005434 | −0.24189 | M | 0.084888 | M | 0.20887 |
| K31866293 | TAK-632 | RAF inhibitor | LZTS1 | | −0.16699 | 0.001344 | 0.031303 | −0.31698 | M | 0.084888 | M | 0.20887 |
| K31866293 | TAK-632 | RAF inhibitor | MCC | colorectal mutant cancer | −0.17802 | 0.000623 | 0.019405 | −0.23874 | M | 0.084888 | M | 0.20887 |
| | | | | protein | | | | | | | | |
| K31866293 | TAK-632 | RAF inhibitor | MEF2C | myocyte-specific enhancer | −0.16874 | 0.001193 | 0.029125 | −0.2599 | M | 0.084888 | M | 0.20887 |
| | | | | factor 2c | | | | | | | | |
| K31866293 | TAK-632 | RAF inhibitor | MIA | melanoma-derived growth | −0.21401 | 3.66E−05 | 0.002959 | −0.32825 | M | 0.084888 | M | 0.20887 |
| | | | | regulatory protein | | | | | | | | |
| K31866293 | TAK-632 | RAF inhibitor | NES | nestin | −0.23999 | 3.42E−06 | 0.000556 | −0.27216 | M | 0.084888 | M | 0.20887 |
| K31866293 | TAK-632 | RAF inhibitor | NFATC2 | nuclear factor of activated t- | −0.21452 | 3.5E−05 | 0.002875 | −0.3235 | M | 0.084888 | M | 0.20887 |
| | | | | cells; cytoplasmic 2 | | | | | | | | |
| K31866293 | TAK-632 | RAF inhibitor | NRROS | transforming growth factor | −0.24743 | 1.65E−06 | 0.000328 | −0.29319 | M | 0.084888 | M | 0.20887 |
| | | | | beta activator lrrc33 | | | | | | | | |
| K31866293 | TAK-632 | RAF inhibitor | PKNOX2 | homeobox protein pknox2 | −0.21417 | 3.61E−05 | 0.002936 | −0.26336 | M | 0.084888 | M | 0.20887 |
| K31866293 | TAK-632 | RAF inhibitor | PLP1 | myelin proteolipid protein | −0.27768 | 6.64E−08 | 3.11E−05 | −0.3682 | M | 0.084888 | M | 0.20887 |
| K31866293 | TAK-632 | RAF inhibitor | PMP2 | myelin p2 protein | −0.24436 | 2.24E−06 | 0.000407 | −0.33352 | M | 0.084888 | M | 0.20887 |
| K31866293 | TAK-632 | RAF inhibitor | PTCRA | pre t-cell antigen receptor | −0.29656 | 7.26E−09 | 6.1E−06 | −0.35336 | M | 0.084888 | M | 0.20887 |
| | | | | alpha | | | | | | | | |
| K31866293 | TAK-632 | RAF inhibitor | RENBP | n-acylglucosamine 2- | −0.19088 | 0.00024 | 0.010525 | −0.27166 | M | 0.084888 | M | 0.20887 |
| | | | | epimerase | | | | | | | | |
| K31866293 | TAK-632 | RAF inhibitor | RGS1 | regulator of g-protein | −0.25298 | 9.43E−07 | 0.000219 | −0.27287 | M | 0.084888 | M | 0.20887 |
| | | | | signaling 1 | | | | | | | | |
| K31866293 | TAK-632 | RAF inhibitor | SCML4 | sex comb on midleg-like | −0.21216 | 4.28E−05 | 0.003297 | −0.26235 | M | 0.084888 | M | 0.20887 |
| | | | | protein 4 | | | | | | | | |
| K31866293 | TAK-632 | RAF inhibitor | SGCD | delta-sarcoglycan | −0.19728 | 0.000145 | 0.007559 | −0.34988 | M | 0.084888 | M | 0.20887 |
| K31866293 | TAK-632 | RAF inhibitor | SOX5 | transcription factor sox-5 | −0.18047 | 0.000522 | 0.017376 | −0.24658 | M | 0.084888 | M | 0.20887 |
| K31866293 | TAK-632 | RAF inhibitor | SRPX | sushi repeat-containing | −0.21441 | 3.53E−05 | 0.002893 | −0.24491 | M | 0.084888 | M | 0.20887 |
| | | | | protein srpx | | | | | | | | |
| K31866293 | TAK-632 | RAF inhibitor | ST6GALNAC3 | alpha-n- | −0.20411 | 8.39E−05 | 0.005213 | −0.28039 | M | 0.084888 | M | 0.20887 |
| | | | | acetylgalactosaminide | | | | | | | | |
| | | | | alpha-2; 6-sialyltransferase 3 | | | | | | | | |
| K31866293 | TAK-632 | RAF inhibitor | TAMALIN | protein tamalin | −0.24933 | 1.36E−06 | 0.000286 | −0.27589 | M | 0.084888 | M | 0.20887 |
| K31866293 | TAK-632 | RAF inhibitor | TIMP3 | metalloproteinase inhibitor 3 | −0.17239 | 0.000928 | 0.024946 | −0.3191 | M | 0.084888 | M | 0.20887 |
| K31866293 | TAK-632 | RAF inhibitor | TMEM229B | transmembrane protein 229b | −0.18695 | 0.000323 | 0.012799 | −0.32241 | M | 0.084888 | M | 0.20887 |
| K31866293 | TAK-632 | RAF inhibitor | TNFRSF 14 | tumor necrosis factor | −0.2052 | 7.67E−05 | 0.004914 | −0.28456 | M | 0.084888 | M | 0.20887 |
| | | | | receptor superfamily | | | | | | | | |
| | | | | member 14 | | | | | | | | |
| K31866293 | TAK-632 | RAF inhibitor | TRPV2 | transient receptor potential | −0.28492 | 2.9E−08 | 1.68E−05 | −0.28755 | M | 0.084888 | M | 0.20887 |
| | | | | cation channel subfamily v | | | | | | | | |
| | | | | member 2 | | | | | | | | |
| K33882852 | ZK-93423 | benzodiazepine receptor | KBTBD6 | kelch repeat and btb | −0.17118 | 0.001129 | 0.028226 | −0.24466 | M | 0.10975 | M | 0.176686 |
| | | agonist | | domain-containing protein 6 | | | | | | | | |
| K37561857 | zardaverine | phosphodiesterase | ABCA8 | atp-binding cassette sub- | −0.19836 | 0.000162 | 0.008113 | −0.32422 | M | 0.178243 | MSL/M | 0.169947 |
| | | inhibitor | | family a member 8 | | | | | | | | |
| K37561857 | zardaverine | phosphodiesterase | ANTXR1 | anthrax toxin receptor 1 | −0.19276 | 0.000249 | 0.010789 | −0.30004 | M | 0.178243 | MSL/M | 0.169947 |
| | | inhibitor | | | | | | | | | | |
| K37561857 | zardaverine | phosphodiesterase | ARHGAP31 | rho gtpase-activating protein | −0.20084 | 0.000133 | 0.007118 | −0.24472 | M | 0.178243 | MSL/M | 0.169947 |
| | | inhibitor | | 31 | | | | | | | | |
| K37561857 | zardaverine | phosphodiesterase | ATOH8 | protein atonal homolog 8 | −0.16205 | 0.002131 | 0.041347 | −0.25099 | M | 0.178243 | MSL/M | 0.169947 |
| | | inhibitor | | | | | | | | | | |
| K37561857 | zardaverine | phosphodiesterase | CALD1 | caldesmon | −0.18504 | 0.000441 | 0.015609 | −0.29655 | M | 0.178243 | MSL/M | 0.169947 |
| | | inhibitor | | | | | | | | | | |
| K37561857 | zardaverine | phosphodiesterase | CNRIP1 | cb1 cannabinoid receptor- | −0.17643 | 0.000814 | 0.022944 | −0.24266 | M | 0.178243 | MSL/M | 0.169947 |
| | | inhibitor | | interacting protein 1 | | | | | | | | |
| K37561857 | zardaverine | phosphodiesterase | CPQ | carboxypeptidase q | −0.17899 | 0.00068 | 0.020495 | −0.29959 | M | 0.178243 | MSL/M | 0.169947 |
| | | inhibitor | | | | | | | | | | |
| K37561857 | zardaverine | phosphodiesterase | CTLA4 | cytotoxic t-lymphocyte | −0.16638 | 0.001607 | 0.034882 | −0.30142 | M | 0.178243 | MSL/M | 0.169947 |
| | | inhibitor | | protein 4 | | | | | | | | |
| K37561857 | zardaverine | phosphodiesterase | DAAM2 | disheveled-associated | −0.20754 | 7.79E−05 | 0.004966 | −0.26364 | M | 0.178243 | MSL/M | 0.169947 |
| | | inhibitor | | activator of morphogenesis 2 | | | | | | | | |
| K37561857 | zardaverine | phosphodiesterase | DSTYK | dual serine/threonine and | −0.18113 | 0.000584 | 0.018637 | −0.32209 | M | 0.178243 | MSL/M | 0.169947 |
| | | inhibitor | | tyrosine protein kinase | | | | | | | | |
| K37561857 | zardaverine | phosphodiesterase | ENOX1 | ecto-nox disulfide-thiol | −0.16359 | 0.001929 | 0.038927 | −0.25369 | M | 0.178243 | MSL/M | 0.169947 |
| | | inhibitor | | exchanger 1 | | | | | | | | |
| K37561857 | zardaverine | phosphodiesterase | FBXL7 | f-box/lrr-repeat protein 7 | −0.20876 | 7.05E−05 | 0.004644 | −0.27171 | M | 0.178243 | MSL/M | 0.169947 |
| | | inhibitor | | | | | | | | | | |
| K37561857 | zardaverine | phosphodiesterase | FERMT2 | fermitin family homolog 2 | −0.16566 | 0.001685 | 0.035933 | −0.2411 | M | 0.178243 | MSL/M | 0.169947 |
| | | inhibitor | | | | | | | | | | |
| K37561857 | zardaverine | phosphodiesterase | GPC6 | glypican-6 | −0.18707 | 0.00038 | 0.014178 | −0.24855 | M | 0.178243 | MSL/M | 0.169947 |
| | | inhibitor | | | | | | | | | | |
| K37561857 | zardaverine | phosphodiesterase | GUCY1A2 | guanylate cyclase soluble | −0.20046 | 0.000137 | 0.007269 | −0.29039 | M | 0.178243 | MSL/M | 0.169947 |
| | | inhibitor | | subunit alpha-2 | | | | | | | | |
| K37561857 | zardaverine | phosphodiesterase | HTRA1 | serine protease htral | −0.22933 | 1.21E−05 | 0.001372 | −0.25932 | M | 0.178243 | MSL/M | 0.169947 |
| | | inhibitor | | | | | | | | | | |
| K37561857 | zardaverine | phosphodiesterase | JAZF1 | juxtaposed with another zinc | −0.16909 | 0.001343 | 0.031285 | −0.27681 | M | 0.178243 | MSL/M | 0.169947 |
| | | inhibitor | | finger protein 1 | | | | | | | | |
| K37561857 | zardaverine | phosphodiesterase | KIRREL1 | kin of irre-like protein 1 | −0.22012 | 2.72E−05 | 0.002408 | −0.28477 | M | 0.178243 | MSL/M | 0.169947 |
| | | inhibitor | | | | | | | | | | |
| K37561857 | zardaverine | phosphodiesterase | LAMA4 | laminin subunit alpha-4 | −0.19769 | 0.00017 | 0.008407 | −0.29128 | M | 0.178243 | MSL/M | 0.169947 |
| | | inhibitor | | | | | | | | | | |
| K37561857 | zardaverine | phosphodiesterase | MMP16 | matrix metalloproteinase-16 | −0.17737 | 0.000762 | 0.022009 | −0.26196 | M | 0.178243 | MSL/M | 0.169947 |
| | | inhibitor | | | | | | | | | | |
| K37561857 | zardaverine | phosphodiesterase | PCDHB7 | protocadherin beta-7 | −0.20994 | 6.41E−05 | 0.004352 | −0.29052 | M | 0.178243 | MSL/M | 0.169947 |
| | | inhibitor | | | | | | | | | | |
| K37561857 | zardaverine | phosphodiesterase | PDE1C | calcium/calmodulin- | −0.19619 | 0.000191 | 0.009081 | −0.31468 | M | 0.178243 | MSL/M | 0.169947 |
| | | inhibitor | | dependent 3′; 5′-cyclic | | | | | | | | |
| | | | | nucleotide | | | | | | | | |
| | | | | phosphodiesterase 1c | | | | | | | | |
| K37561857 | zardaverine | phosphodiesterase | PDE7B | camp-specific 3′; 5′-cyclic | −0.17821 | 0.000718 | 0.021216 | −0.26066 | M | 0.178243 | MSL/M | 0.169947 |
| | | inhibitor | | phosphodiesterase 7b | | | | | | | | |
| K37561857 | zardaverine | phosphodiesterase | PYGO1 | pygopus homolog 1 | −0.18995 | 0.000307 | 0.012374 | −0.24375 | M | 0.178243 | MSL/M | 0.169947 |
| | | inhibitor | | | | | | | | | | |
| K37561857 | zardaverine | phosphodiesterase | RASSF8 | ras association domain- | −0.19687 | 0.000182 | 0.00877 | −0.24749 | M | 0.178243 | MSL/M | 0.169947 |
| | | inhibitor | | containing protein 8 | | | | | | | | |
| K37561857 | zardaverine | phosphodiesterase | RECK | reversion-inducing cysteine- | −0.19612 | 0.000192 | 0.00911 | −0.25912 | M | 0.178243 | MSL/M | 0.169947 |
| | | inhibitor | | rich protein with kazal | | | | | | | | |
| | | | | motifs | | | | | | | | |
| K37561857 | zardaverine | phosphodiesterase | RHOJ | rho-related gtp-binding | −0.22677 | 1.52E−05 | 0.001614 | −0.27811 | M | 0.178243 | MSL/M | 0.169947 |
| | | inhibitor | | protein rhoj | | | | | | | | |
| K37561857 | zardaverine | phosphodiesterase | SH3PXD2B | sh3 and px domain- | −0.20235 | 0.000118 | 0.006577 | −0.26621 | M | 0.178243 | MSL/M | 0.169947 |
| | | inhibitor | | containing protein 2b | | | | | | | | |
| K37561857 | zardaverine | phosphodiesterase | SHC4 | shc-transforming protein 4 | −0.21121 | 5.77E−05 | 0.004046 | −0.32295 | M | 0.178243 | MSL/M | 0.169947 |
| | | inhibitor | | | | | | | | | | |
| K37561857 | zardaverine | phosphodiesterase | SHISA4 | protein shisa-4 | −0.16891 | 0.001358 | 0.031518 | −0.25725 | M | 0.178243 | MSL/M | 0.169947 |
| | | inhibitor | | | | | | | | | | |
| K37561857 | zardaverine | phosphodiesterase | SMIM10 | small integral membrane | −0.16019 | 0.002399 | 0.044354 | −0.24592 | M | 0.178243 | MSL/M | 0.169947 |
| | | inhibitor | | protein 10 | | | | | | | | |
| K37561857 | zardaverine | phosphodiesterase | SPARC | sparc | −0.20897 | 6.93E−05 | 0.004588 | −0.2752 | M | 0.178243 | MSL/M | 0.169947 |
| | | inhibitor | | | | | | | | | | |
| K37561857 | zardaverine | phosphodiesterase | SRPX | sushi repeat-containing | −0.19884 | 0.000156 | 0.007908 | −0.26039 | M | 0.178243 | MSL/M | 0.169947 |
| | | inhibitor | | protein srpx | | | | | | | | |
| K37561857 | zardaverine | phosphodiesterase | ST3GAL2 | cmp-n-acetylneuraminate- | −0.17048 | 0.001222 | 0.029555 | −0.24425 | M | 0.178243 | MSL/M | 0.169947 |
| | | inhibitor | | beta-galactosamide-alpha- | | | | | | | | |
| | | | | 2; 3-sialyltransferase 2 | | | | | | | | |
| K37561857 | zardaverine | phosphodiesterase | SYDE1 | rho gtpase-activating protein | −0.21693 | 3.57E−05 | 0.002914 | −0.24653 | M | 0.178243 | MSL/M | 0.169947 |
| | | inhibitor | | syde1 | | | | | | | | |
| K37561857 | zardaverine | phosphodiesterase | TMTC1 | protein o-mannosyl- | −0.15846 | 0.002677 | 0.047273 | −0.32165 | M | 0.178243 | MSL/M | 0.169947 |
| | | inhibitor | | transferase tmtc1 | | | | | | | | |
| K37561857 | zardaverine | phosphodiesterase | TUB | tubby protein homolog | −0.22575 | 1.66E−05 | 0.001718 | −0.29453 | M | 0.178243 | MSL/M | 0.169947 |
| | | inhibitor | | | | | | | | | | |
| K37561857 | zardaverine | phosphodiesterase | ZCCHC24 | | −0.17043 | 0.001227 | 0.029623 | −0.29506 | M | 0.178243 | MSL/M | 0.169947 |
| | | inhibitor | | | | | | | | | | |
| K37561857 | zardaverine | phosphodiesterase | ZEB2 | zinc finger e-box-binding | −0.19207 | 0.000262 | 0.011141 | −0.25775 | M | 0.178243 | MSL/M | 0.169947 |
| | | inhibitor | | homeobox 2 | | | | | | | | |
| K40109029 | SB-505124 | ALK tyrosine kinase | CTLA4 | cytotoxic t-lymphocyte | −0.22798 | 1.03E−05 | 0.001224 | −0.23757 | M | 0.256196 | M | 0.168251 |
| | | receptor inhibitor | | protein 4 | | | | | | | | |
| K40109029 | SB-505124 | ALK tyrosine kinase | GASK1B | golgi-associated kinase 1b | −0.24597 | 1.85E−06 | 0.000357 | −0.25533 | M | 0.256196 | M | 0.168251 |
| | | receptor inhibitor | | | | | | | | | | |
| K40109029 | SB-505124 | ALK tyrosine kinase | GHR | growth hormone receptor | −0.26955 | 1.58E−07 | 5.93E−05 | −0.24933 | M | 0.256196 | M | 0.168251 |
| | | receptor inhibitor | | | | | | | | | | |
| K40109029 | SB-505124 | ALK tyrosine kinase | HMCN1 | hemicentin-1 | −0.3493 | 5.7E−12 | 2.97E−08 | −0.24951 | M | 0.256196 | M | 0.168251 |
| | | receptor inhibitor | | | | | | | | | | |
| K40109029 | SB-505124 | ALK tyrosine kinase | IL16 | pro-interleukin-16 | −0.28479 | 2.81E−08 | 1.65E−05 | −0.30037 | M | 0.256196 | M | 0.168251 |
| | | receptor inhibitor | | | | | | | | | | |
| K40109029 | SB-505124 | ALK tyrosine kinase | IRF4 | interferon regulatory factor | −0.2899 | 1.54E−08 | 1.07E−05 | −0.25946 | M | 0.256196 | M | 0.168251 |
| | | receptor inhibitor | | 4 | | | | | | | | |
| K40109029 | SB-505124 | ALK tyrosine kinase | PARD6G | partitioning defective 6 | −0.27305 | 1.07E−07 | 4.47E−05 | −0.26637 | M | 0.256196 | M | 0.168251 |
| | | receptor inhibitor | | homolog gamma | | | | | | | | |
| K40109029 | SB-505124 | ALK tyrosine kinase | TMEM229B | transmembrane protein 229b | −0.16465 | 0.00155 | 0.034145 | −0.25527 | M | 0.256196 | M | 0.168251 |
| | | receptor inhibitor | | | | | | | | | | |
| K46386702 | ARRY-334543 | EGFR inhibitor | ALOX5 | polyunsaturated fatty acid 5- | −0.21787 | 2.75E−05 | 0.00243 | −0.35167 | IM | −0.05992 | IM | −0.03109 |
| | | | | lipoxygenase | | | | | | | | |
| K46386702 | ARRY-334543 | EGFR inhibitor | ANKRD65 | ankyrin repeat domain- | −0.27871 | 6.42E−08 | 3.04E−05 | −0.27596 | IM | −0.05992 | IM | −0.03109 |
| | | | | containing protein 65 | | | | | | | | |
| K46386702 | ARRY-334543 | EGFR inhibitor | ARHGEF4 | rho guanine nucleotide | −0.18456 | 0.000401 | 0.01467 | −0.24294 | IM | −0.05992 | IM | −0.03109 |
| | | | | exchange factor 4 | | | | | | | | |
| K46386702 | ARRY-334543 | EGFR inhibitor | BCL2L14 | apoptosis facilitator bcl-2- | −0.18512 | 0.000385 | 0.014282 | −0.2448 | IM | −0.05992 | IM | −0.03109 |
| | | | | like protein 14 | | | | | | | | |
| K46386702 | ARRY-334543 | EGFR inhibitor | COL17A1 | collagen alpha-1 | −0.2911 | 1.53E−08 | 1.06E−05 | −0.36549 | IM | −0.05992 | IM | −0.03109 |
| K46386702 | ARRY-334543 | EGFR inhibitor | CXCL16 | c-x-c motif chemokine 16 | −0.25673 | 6.88E−07 | 0.000175 | −0.38672 | IM | −0.05992 | IM | −0.03109 |
| K46386702 | ARRY-334543 | EGFR inhibitor | CYP4B1 | cytochrome p450 4b1 | −0.20516 | 8.06E−05 | 0.005075 | −0.33476 | IM | −0.05992 | IM | −0.03109 |
| K46386702 | ARRY-334543 | EGFR inhibitor | DAPP1 | dual adapter for | −0.33215 | 7.99E−11 | 2.05E−07 | −0.38968 | IM | −0.05992 | IM | −0.03109 |
| | | | | phosphotyrosine and 3- | | | | | | | | |
| | | | | phosphotyrosine and 3- | | | | | | | | |
| | | | | phosphoinositide | | | | | | | | |
| K46386702 | ARRY-334543 | EGFR inhibitor | DENND1C | denn domain-containing | −0.28544 | 2.97E−08 | 1.71E−05 | −0.40436 | IM | −0.05992 | IM | −0.03109 |
| | | | | protein 1c | | | | | | | | |
| K46386702 | ARRY-334543 | EGFR inhibitor | DENND2D | denn domain-containing | −0.2154 | 3.41E−05 | 0.002822 | −0.28939 | IM | −0.05992 | IM | −0.03109 |
| | | | | protein 2d | | | | | | | | |
| K46386702 | ARRY-334543 | EGFR inhibitor | DSG3 | desmoglein-3 | −0.3574 | 2.08E−12 | 1.44E−08 | −0.30164 | IM | −0.05992 | IM | −0.03109 |
| K46386702 | ARRY-334543 | EGFR inhibitor | FGD3 | fyve; rhogef and ph domain- | −0.28765 | 2.3E−08 | 1.43E−05 | −0.38187 | IM | −0.05992 | IM | −0.03109 |
| | | | | containing protein 3 | | | | | | | | |
| K46386702 | ARRY-334543 | EGFR inhibitor | HSH2D | hematopoietic sh2 domain- | −0.17983 | 0.000566 | 0.018269 | −0.35709 | IM | −0.05992 | IM | −0.03109 |
| | | | | containing protein | | | | | | | | |
| K46386702 | ARRY-334543 | EGFR inhibitor | ITGB4 | integrin beta-4 | −0.3474 | 9.18E−12 | 4.18E−08 | −0.43913 | IM | −0.05992 | IM | −0.03109 |
| K46386702 | ARRY-334543 | EGFR inhibitor | KRT16 | keratin; type i cytoskeletal 16 | −0.36969 | 3.13E−13 | 3.61E−09 | −0.34942 | IM | −0.05992 | IM | −0.03109 |
| K46386702 | ARRY-334543 | EGFR inhibitor | KRT17 | keratin; type i cytoskeletal 17 | −0.33817 | 3.45E−11 | 1.12E−07 | −0.32262 | IM | −0.05992 | IM | −0.03109 |
| K46386702 | ARRY-334543 | EGFR inhibitor | KRT5 | keratin; type ii cytoskeletal 5 | −0.33128 | 9.02E−11 | 2.25E−07 | −0.26495 | IM | −0.05992 | IM | −0.03109 |
| K46386702 | ARRY-334543 | EGFR inhibitor | LGALS9 | galectin-9 | −0.18266 | 0.000461 | 0.016061 | −0.30803 | IM | −0.05992 | IM | −0.03109 |
| K46386702 | ARRY-334543 | EGFR inhibitor | LPAR5 | lysophosphatidic acid | −0.31234 | 1.12E−09 | 1.51E−06 | −0.35547 | IM | −0.05992 | IM | −0.03109 |
| | | | | receptor 5 | | | | | | | | |
| K46386702 | ARRY-334543 | EGFR inhibitor | PTAFR | platelet-activating factor | −0.33877 | 3.17E−11 | 1.04E−07 | −0.4259 | IM | −0.05992 | IM | −0.03109 |
| | | | | receptor | | | | | | | | |
| K46386702 | ARRY-334543 | EGFR inhibitor | PTPN6 | tyrosine-protein phosphatase | −0.33498 | 5.4E−11 | 1.51E−07 | −0.38814 | IM | −0.05992 | IM | −0.03109 |
| | | | | non-receptor type 6 | | | | | | | | |
| K46386702 | ARRY-334543 | EGFR inhibitor | RASGEF1B | ras-gef domain-containing | −0.15931 | 0.0023 | 0.043244 | −0.24753 | IM | −0.05992 | IM | −0.03109 |
| | | | | family member 1b | | | | | | | | |
| K46386702 | ARRY-334543 | EGFR inhibitor | S100A8 | protein s100-a8 | −0.18814 | 0.000307 | 0.012371 | −0.2876 | IM | −0.05992 | IM | −0.03109 |
| K46386702 | ARRY-334543 | EGFR inhibitor | SH3BP1 | bargin-related | −0.28775 | 2.28E−08 | 1.41E−05 | −0.30924 | IM | −0.05992 | IM | −0.03109 |
| K46386702 | ARRY-334543 | EGFR inhibitor | SIRPB2 | signal-regulatory protein | −0.17104 | 0.001052 | 0.026995 | −0.2414 | IM | −0.05992 | IM | −0.03109 |
| | | | | beta-2 | | | | | | | | |
| K46386702 | ARRY-334543 | EGFR inhibitor | TEAD3 | transcriptional enhancer | −0.23145 | 8.15E−06 | 0.001036 | −0.26729 | IM | −0.05992 | IM | −0.03109 |
| | | | | factor tef-5 | | | | | | | | |
| K46386702 | ARRY-334543 | EGFR inhibitor | TNFSF10 | tumor necrosis factor ligand | −0.25726 | 6.52E−07 | 0.000168 | −0.28575 | IM | −0.05992 | IM | −0.03109 |
| | | | | superfamily member 10 | | | | | | | | |
| K46386702 | ARRY-334543 | EGFR inhibitor | TRAF3IP3 | traf3-interacting jnk- | −0.17141 | 0.001026 | 0.026579 | −0.26103 | IM | −0.05992 | IM | −0.03109 |
| | | | | activating modulator | | | | | | | | |
| K46386702 | ARRY-334543 | EGFR inhibitor | UNC13D | protein unc-13 homolog d | −0.20271 | 9.83E−05 | 0.005808 | −0.29889 | IM | −0.05992 | IM | −0.03109 |
| K46386702 | ARRY-334543 | EGFR inhibitor | VIPR1 | vasoactive intestinal | −0.16628 | 0.001454 | 0.032847 | −0.26419 | IM | −0.05992 | IM | −0.03109 |
| | | | | polypeptide receptor 1 | | | | | | | | |
| K46386702 | ARRY-334543 | EGFR inhibitor | ZMYND15 | zinc finger mynd domain- | −0.23897 | 4.02E−06 | 0.000622 | −0.26625 | IM | −0.05992 | IM | −0.03109 |
| | | | | containing protein 15 | | | | | | | | |
| K49294207 | BIBU-1361 | EGFR inhibitor | COL17A1 | collagen alpha-1 | −0.24453 | 2.67E−06 | 0.000463 | −0.3684 | IM | −0.06618 | IM | −0.00947 |
| K49294207 | BIBU-1361 | EGFR inhibitor | CXCL16 | c-x-c motif chemokine 16 | −0.20584 | 8.35E−05 | 0.005199 | −0.36341 | IM | −0.06618 | IM | −0.00947 |
| K49294207 | BIBU-1361 | EGFR inhibitor | DAPP1 | dual adapter for | −0.27615 | 1.01E−07 | 4.26E−05 | −0.40028 | IM | −0.06618 | IM | −0.00947 |
| | | | | phosphotyrosine and 3- | | | | | | | | |
| | | | | phosphotyrosine and 3- | | | | | | | | |
| | | | | phosphoinositide | | | | | | | | |
| K49294207 | BIBU-1361 | EGFR inhibitor | DENND1C | denn domain-containing | −0.27792 | 8.29E−08 | 3.67E−05 | −0.38121 | IM | −0.06618 | IM | −0.00947 |
| | | | | protein 1c | | | | | | | | |
| K49294207 | BIBU-1361 | EGFR inhibitor | DENND2D | denn domain-containing | −0.19684 | 0.000171 | 0.008425 | −0.24489 | IM | −0.06618 | IM | −0.00947 |
| | | | | protein 2d | | | | | | | | |
| K49294207 | BIBU-1361 | EGFR inhibitor | DSG3 | desmoglein-3 | −0.28748 | 2.8E−08 | 1.65E−05 | −0.33781 | IM | −0.06618 | IM | −0.00947 |
| K49294207 | BIBU-1361 | EGFR inhibitor | FGD3 | fyve; rhogef and ph domain- | −0.23197 | 8.72E−06 | 0.001089 | −0.31506 | IM | −0.06618 | IM | −0.00947 |
| | | | | containing protein 3 | | | | | | | | |
| K49294207 | BIBU-1361 | EGFR inhibitor | HSH2D | hematopoietic sh2 domain- | −0.17298 | 0.000983 | 0.02586 | −0.34629 | IM | −0.06618 | IM | −0.00947 |
| | | | | containing protein | | | | | | | | |
| K49294207 | BIBU-1361 | EGFR inhibitor | ITGB4 | integrin beta-4 | −0.30544 | 3.28E−09 | 3.33E−06 | −0.39282 | IM | −0.06618 | IM | −0.00947 |
| K49294207 | BIBU-1361 | EGFR inhibitor | KRT16 | keratin; type i cytoskeletal 16 | −0.26326 | 4.04E−07 | 0.000119 | −0.42928 | IM | −0.06618 | IM | −0.00947 |
| K49294207 | BIBU-1361 | EGFR inhibitor | KRT17 | keratin; type i cytoskeletal 17 | −0.27269 | 1.47E−07 | 5.65E−05 | −0.31196 | IM | −0.06618 | IM | −0.00947 |
| K49294207 | BIBU-1361 | EGFR inhibitor | KRT5 | keratin; type ii cytoskeletal 5 | −0.25941 | 6.02E−07 | 0.000159 | −0.25585 | IM | −0.06618 | IM | −0.00947 |
| K49294207 | BIBU-1361 | EGFR inhibitor | LPAR5 | lysophosphatidic acid | −0.26692 | 2.74E−07 | 8.96E−05 | −0.33857 | IM | −0.06618 | IM | −0.00947 |
| | | | | receptor 5 | | | | | | | | |
| K49294207 | BIBU-1361 | EGFR inhibitor | PTAFR | platelet-activating factor | −0.28153 | 5.53E−08 | 2.72E−05 | −0.38521 | IM | −0.06618 | IM | −0.00947 |
| | | | | receptor | | | | | | | | |
| K49294207 | BIBU-1361 | EGFR inhibitor | PTPN6 | tyrosine-protein phosphatase | −0.32344 | 3.27E−10 | 5.85E−07 | −0.40666 | IM | −0.06618 | IM | −0.00947 |
| | | | | non-receptor type 6 | | | | | | | | |
| K49294207 | BIBU-1361 | EGFR inhibitor | SH3BP1 | bargin-related | −0.1607 | 0.002225 | 0.042431 | −0.24329 | IM | −0.06618 | IM | −0.00947 |
| K49294207 | BIBU-1361 | EGFR inhibitor | SIRPB2 | signal-regulatory protein | −0.18684 | 0.000365 | 0.013828 | −0.25174 | IM | −0.06618 | IM | −0.00947 |
| | | | | beta-2 | | | | | | | | |
| K49294207 | BIBU-1361 | EGFR inhibitor | TNFSF10 | tumor necrosis factor ligand | −0.2681 | 2.42E−07 | 8.16E−05 | −0.25405 | IM | −0.06618 | IM | −0.00947 |
| | | | | superfamily member 10 | | | | | | | | |
| K49294207 | BIBU-1361 | EGFR inhibitor | UNC13D | protein unc-13 homolog d | −0.1927 | 0.000235 | 0.010393 | −0.26505 | IM | −0.06618 | IM | −0.00947 |
| K49294207 | BIBU-1361 | EGFR inhibitor | VSIR | v-type immunoglobulin | −0.16145 | 0.00212 | 0.041224 | −0.24964 | IM | −0.06618 | IM | −0.00947 |
| | | | | domain-containing | | | | | | | | |
| | | | | suppressor of t-cell | | | | | | | | |
| | | | | activation | | | | | | | | |
| K49294207 | BIBU-1361 | EGFR inhibitor | ZMYND15 | zinc finger mynd domain- | −0.17407 | 0.000911 | 0.024644 | −0.29204 | IM | −0.06618 | IM | −0.00947 |
| | | | | containing protein 15 | | | | | | | | |
| K49328571 | dasatinib | Bcr-Abl kinase | ANKRD33B | ankyrin repeat domain- | −0.27464 | 1.24E−07 | 4.98E−05 | −0.29073 | IM | −0.17261 | IM | −0.06188 |
| | | inhibitor; ephrin | | containing protein 33b | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | APOL1 | apolipoprotein 11 | −0.2592 | 6.38E−07 | 0.000165 | −0.25243 | IM | −0.17261 | IM | −0.06188 |
| | | inhibitor; ephrin | | | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | BIRC3 | baculoviral iap repeat- | −0.23852 | 4.88E−06 | 0.000715 | −0.27806 | IM | −0.17261 | IM | −0.06188 |
| | | inhibitor; ephrin | | containing protein 3 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | C15orf48 | normal mucosa of | −0.16281 | 0.00197 | 0.039424 | −0.27745 | IM | −0.17261 | IM | −0.06188 |
| | | inhibitor; ephrin | | esophagus-specific gene 1 | | | | | | | | |
| | | inhibitor; KIT inhibitor; | | protein | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | CATSPER1 | cation channel sperm- | −0.1617 | 0.002117 | 0.041188 | −0.27935 | IM | −0.17261 | IM | −0.06188 |
| | | inhibitor; ephrin | | associated protein 1 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | CLEC4E | c-type lectin domain family | −0.2357 | 6.36E−06 | 0.000866 | −0.26912 | IM | −0.17261 | IM | −0.06188 |
| | | inhibitor; ephrin | | 4 member e | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | CLIP4 | cap-gly domain-containing | −0.20482 | 9.28E−05 | 0.005573 | −0.26572 | IM | −0.17261 | IM | −0.06188 |
| | | inhibitor; ephrin | | linker protein 4 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | DRAM1 | dna damage-regulated | −0.16332 | 0.001905 | 0.038619 | −0.28159 | IM | −0.17261 | IM | −0.06188 |
| | | inhibitor; ephrin | | autophagy modulator | | | | | | | | |
| | | inhibitor; KIT inhibitor; | | protein 1 | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | IGFBP7 | insulin-like growth factor- | −0.21189 | 5.19E−05 | 0.003772 | −0.24301 | IM | −0.17261 | IM | −0.06188 |
| | | inhibitor; ephrin | | binding protein 7 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | IL15RA | interleukin-15 receptor | −0.18942 | 0.000307 | 0.012374 | −0.23909 | IM | −0.17261 | IM | −0.06188 |
| | | inhibitor; ephrin | | subunit alpha | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | IL32 | | −0.20068 | 0.000129 | 0.006977 | −0.2635 | IM | −0.17261 | IM | −0.06188 |
| | | inhibitor; ephrin | | | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | LTB | lymphotoxin-beta | −0.16377 | 0.00185 | 0.037996 | −0.2851 | IM | −0.17261 | IM | −0.06188 |
| | | inhibitor; ephrin | | | | | | | | | | |
| | | inhibitor; KIT inhibitor, | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | RAC2 | ras-related c3 botulinum | −0.17604 | 0.000808 | 0.022845 | −0.26127 | IM | −0.17261 | IM | −0.06188 |
| | | inhibitor; ephrin | | toxin substrate 2 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | SORCS2 | vps10 domain-containing | −0.18499 | 0.000426 | 0.015275 | −0.29236 | IM | −0.17261 | IM | −0.06188 |
| | | inhibitor; ephrin | | receptor sorcs2 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | SYTL3 | synaptotagmin-like protein 3 | −0.20501 | 9.13E−05 | 0.005515 | −0.30285 | IM | −0.17261 | IM | −0.06188 |
| | | inhibitor; ephrin | | | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | TNF | tumor necrosis factor | −0.30221 | 5.12E−09 | 4.68E−06 | −0.34193 | IM | −0.17261 | IM | −0.06188 |
| | | inhibitor; ephrin | | | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | TNFAIP3 | tumor necrosis factor alpha- | −0.21998 | 2.61E−05 | 0.002344 | −0.25472 | IM | −0.17261 | IM | −0.06188 |
| | | inhibitor; ephrin | | induced protein 3 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | TNF AIP8 | tumor necrosis factor alpha- | −0.22123 | 2.34E−05 | 0.002171 | −0.24852 | IM | −0.17261 | IM | −0.06188 |
| | | inhibitor; ephrin | | induced protein 8 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K49328571 | dasatinib | Bcr-Abl kinase | TNFSF10 | tumor necrosis factor ligand | −0.23745 | 5.4E−06 | 0.000768 | −0.24386 | IM | −0.17261 | IM | −0.06188 |
| | | inhibitor; ephrin | | superfamily member 10 | | | | | | | | |
| | | inhibitor; KIT inhibitor; | | | | | | | | | | |
| | | PDGFR tyrosine kinase | | | | | | | | | | |
| | | receptor inhibitor; src | | | | | | | | | | |
| | | inhibitor; tyrosine | | | | | | | | | | |
| | | kinase inhibitor | | | | | | | | | | |
| K52256627 | chlorhexidine | membrane integrity | DKK2 | dickkopf-related protein 2 | −0.18693 | 0.000342 | 0.01328 | −0.24772 | M | 0.14564 | M | 0.086625 |
| | | inhibitor | | | | | | | | | | |
| K52256627 | chlorhexidine | membrane integrity | DLX6 | homeobox protein dlx-6 | −0.16972 | 0.00117 | 0.028791 | −0.23759 | M | 0.14564 | M | 0.086625 |
| | | inhibitor | | | | | | | | | | |
| K52256627 | chlorhexidine | membrane integrity | GPC3 | glypican-3 | −0.18758 | 0.000326 | 0.012879 | −0.26201 | M | 0.14564 | M | 0.086625 |
| | | inhibitor | | | | | | | | | | |
| K52256627 | chlorhexidine | membrane integrity | PDZD4 | pdz domain-containing | −0.27835 | 6.97E−08 | 3.24E−05 | −0.27238 | M | 0.14564 | M | 0.086625 |
| | | inhibitor | | protein 4 | | | | | | | | |
| K52256627 | chlorhexidine | membrane integrity | PLPP7 | inactive phospholipid | −0.2006 | 0.000119 | 0.006608 | −0.25559 | M | 0.14564 | M | 0.086625 |
| | | inhibitor | | phosphatase 7 | | | | | | | | |
| K52256627 | chlorhexidine | membrane integrity | RBPMS2 | rna-binding protein with | −0.2576 | 6.52E−07 | 0.000168 | −0.26519 | M | 0.14564 | M | 0.086625 |
| | | inhibitor | | multiple splicing 2 | | | | | | | | |
| K52256627 | chlorhexidine | membrane integrity | SCARF2 | scavenger receptor class f | −0.21162 | 4.82E−05 | 0.003581 | −0.27678 | M | 0.14564 | M | 0.086625 |
| | | inhibitor | | member 2 | | | | | | | | |
| K52256627 | chlorhexidine | membrane integrity | SMO | smoothened homolog | −0.23997 | 3.76E−06 | 0.000595 | −0.26163 | M | 0.14564 | M | 0.086625 |
| | | inhibitor | | | | | | | | | | |
| K54395039 | PR-619 | DUB inhibitor | ANXA6 | annexin a6 | −0.17686 | 0.000665 | 0.020214 | −0.29594 | M | 0.099296 | M | 0.056745 |
| K54395039 | PR-619 | DUB inhibitor | ATP8B2 | phospholipid-transporting | −0.16357 | 0.001666 | 0.035668 | −0.24748 | M | 0.099296 | M | 0.056745 |
| | | | | atpase id | | | | | | | | |
| K54395039 | PR-619 | DUB inhibitor | C3orf18 | similar to riken cdna | −0.22346 | 1.55E−05 | 0.001641 | −0.24104 | M | 0.099296 | M | 0.056745 |
| | | | | 6430571113 gene; similar to | | | | | | | | |
| | | | | g20 protein | | | | | | | | |
| K54395039 | PR-619 | DUB inhibitor | DLG4 | disks large homolog 4- | −0.15763 | 0.002458 | 0.044954 | −0.30239 | M | 0.099296 | M | 0.056745 |
| | | | | related | | | | | | | | |
| K54395039 | PR-619 | DUB inhibitor | FBXL7 | f-box/lrr-repeat protein 7 | −0.21952 | 2.21E−05 | 0.002085 | −0.23699 | M | 0.099296 | M | 0.056745 |
| K54395039 | PR-619 | DUB inhibitor | FMNL3 | formin-like protein 3 | −0.23918 | 3.59E−06 | 0.000577 | −0.24778 | M | 0.099296 | M | 0.056745 |
| K54395039 | PR-619 | DUB inhibitor | FOXO3B | forkhead box protein o3b- | −0.15492 | 0.002923 | 0.049814 | −0.25412 | M | 0.099296 | M | 0.056745 |
| | | | | related | | | | | | | | |
| K54395039 | PR-619 | DUB inhibitor | PEAK1 | inactive tyrosine-protein | −0.18788 | 0.000295 | 0.012059 | −0.28406 | M | 0.099296 | M | 0.056745 |
| | | | | kinase peak 1 | | | | | | | | |
| K54395039 | PR-619 | DUB inhibitor | REEP2 | receptor expression- | −0.16837 | 0.001205 | 0.029304 | −0.25182 | M | 0.099296 | M | 0.056745 |
| | | | | enhancing protein 2 | | | | | | | | |
| K54395039 | PR-619 | DUB inhibitor | SHANK1 | sh3 and multiple ankyrin | −0.19641 | 0.000153 | 0.00781 | −0.30708 | M | 0.099296 | M | 0.056745 |
| | | | | repeat domains protein 1 | | | | | | | | |
| K54634444 | artesunate | DNA synthesis inhibitor | EMILIN3 | emilin-3 | −0.18551 | 0.000347 | 0.013375 | −0.27206 | M | 0.132469 | M | 0.077482 |
| K54634444 | artesunate | DNA synthesis inhibitor | MSI1 | rna-binding protein musashi | −0.16232 | 0.001784 | 0.037183 | −0.27116 | M | 0.132469 | M | 0.077482 |
| | | | | homolog 1 | | | | | | | | |
| K54634444 | artesunate | DNA synthesis inhibitor | PIANP | pilr alpha-associated neural | −0.20638 | 6.64E−05 | 0.004457 | −0.25949 | M | 0.132469 | M | 0.077482 |
| | | | | protein | | | | | | | | |
| K58529924 | ONC201 | AKT inhibitor; MAP | DIRAS1 | gtp-binding protein di-ras 1 | −0.16074 | 0.002488 | 0.045255 | −0.25377 | M | 0.050772 | M | 0.104631 |
| | | kinase inhibitor | | | | | | | | | | |
| K58529924 | ONC201 | AKT inhibitor; MAP | DVL2 | segment polarity protein | −0.20955 | 7.44E−05 | 0.004816 | −0.25991 | M | 0.050772 | M | 0.104631 |
| | | kinase inhibitor | | dishevelled homolog dvl-2 | | | | | | | | |
| K58529924 | ONC201 | AKT inhibitor; MAP | MLLT1 | protein enl | −0.16029 | 0.00256 | 0.046038 | −0.28115 | M | 0.050772 | M | 0.104631 |
| | | kinase inhibitor | | | | | | | | | | |
| K60130390 | UNC0642 | histone lysine | ISG15 | ubiquitin-like protein isg15 | −0.17866 | 0.000786 | 0.022451 | −0.295 | IM | −0.0704 | IM | −0.1219 |
| | | methyltransferase | | | | | | | | | | |
| | | inhibitor | | | | | | | | | | |
| K60443845 | chlormidazole | fungal lanosterol | ADAM11 | disintegrin and | −0.19749 | 0.000192 | 0.00911 | −0.24658 | M | 0.20157 | M | 0.161226 |
| | | demethylase inhibitor | | metalloproteinase domain- | | | | | | | | |
| | | | | containing protein 11 | | | | | | | | |
| K60443845 | chlormidazole | fungal lanosterol | MPP2 | forkhead box protein ml | −0.20114 | 0.000145 | 0.007549 | −0.26951 | M | 0.20157 | M | 0.161226 |
| | | demethylase inhibitor | | | | | | | | | | |
| K60443845 | chlormidazole | fungal lanosterol | RCOR2 | rest corepressor 2 | −0.1723 | 0.001172 | 0.028828 | −0.34902 | M | 0.20157 | M | 0.161226 |
| | | demethylase inhibitor | | | | | | | | | | |
| K62200014 | anagrelide | phosphodiesterase | CTLA4 | cytotoxic t-lymphocyte | −0.17003 | 0.00124 | 0.029809 | −0.27496 | M | 0.152247 | M | 0.254063 |
| | | inhibitor | | protein 4 | | | | | | | | |
| K62200014 | anagrelide | phosphodiesterase | FBXL7 | f-box/lrr-repeat protein 7 | −0.24722 | 2.19E−06 | 0.000401 | −0.25199 | M | 0.152247 | M | 0.254063 |
| | | inhibitor | | | | | | | | | | |
| K62200014 | anagrelide | phosphodiesterase | GYPC | glycophorin-c | −0.20748 | 7.65E−05 | 0.004905 | −0.26615 | M | 0.152247 | M | 0.254063 |
| | | inhibitor | | | | | | | | | | |
| K62200014 | anagrelide | phosphodiesterase | PIP4K2B | phosphatidylinositol 5- | −0.16979 | 0.001261 | 0.03013 | −0.25046 | M | 0.152247 | M | 0.254063 |
| | | inhibitor | | phosphate 4-kinase type-2 | | | | | | | | |
| | | | | beta | | | | | | | | |
| K62200014 | anagrelide | phosphodiesterase | PYGO1 | pygopus homolog 1 | −0.16124 | 0.002213 | 0.042288 | −0.24629 | M | 0.152247 | M | 0.254063 |
| | | inhibitor | | | | | | | | | | |
| K62213621 | dihydroartemisinin | antimalarial agent | ACTR3B | actin-related protein 3b- | −0.18073 | 0.00056 | 0.018138 | −0.26955 | M | 0.185453 | M | 0.134506 |
| | | | | related | | | | | | | | |
| K62213621 | dihydroartemisinin | antimalarial agent | EMILIN3 | emilin-3 | −0.18969 | 0.00029 | 0.011906 | −0.25533 | M | 0.185453 | M | 0.134506 |
| K62213621 | dihydroartemisinin | antimalarial agent | FAM171A2 | protein fam171a2 | −0.19222 | 0.000239 | 0.010504 | −0.2432 | M | 0.185453 | M | 0.134506 |
| K62213621 | dihydroartemisinin | antimalarial agent | GALNT17 | polypeptide n- | −0.18095 | 0.000551 | 0.017968 | −0.2462 | M | 0.185453 | M | 0.134506 |
| | | | | acetylgalactosaminyltransferase- | | | | | | | | |
| | | | | like 6 | | | | | | | | |
| K62213621 | dihydroartemisinin | antimalarial agent | KCNH2 | potassium voltage-gated | −0.19548 | 0.000186 | 0.008907 | −0.25898 | M | 0.185453 | M | 0.134506 |
| | | | | channel subfamily h | | | | | | | | |
| | | | | member 2 | | | | | | | | |
| K62213621 | dihydroartemisinin | antimalarial agent | MEX3A | rna-binding protein mex3a | −0.23019 | 9.97E−06 | 0.001196 | −0.31173 | M | 0.185453 | M | 0.134506 |
| K62213621 | dihydroartemisinin | antimalarial agent | MPP2 | forkhead box protein m1 | −0.2072 | 7.3E−05 | 0.004756 | −0.2677 | M | 0.185453 | M | 0.134506 |
| K62213621 | dihydroartemisinin | antimalarial agent | MSI1 | rna-binding protein musashi | −0.21692 | 3.23E−05 | 0.002717 | −0.40243 | M | 0.185453 | M | 0.134506 |
| | | | | homolog 1 | | | | | | | | |
| K62213621 | dihydroartemisinin | antimalarial agent | NYNRIN | protein nynrin | −0.17219 | 0.001021 | 0.026494 | −0.3429 | M | 0.185453 | M | 0.134506 |
| K62213621 | dihydroartemisinin | antimalarial agent | PLANP | pilr alpha-associated neural | −0.22657 | 1.38E−05 | 0.001512 | −0.25594 | M | 0.185453 | M | 0.134506 |
| | | | | protein | | | | | | | | |
| K62213621 | dihydroartemisinin | antimalarial agent | RASL10B | ras-like protein family | −0.18815 | 0.000325 | 0.012846 | −0.24285 | M | 0.185453 | M | 0.134506 |
| | | | | member 10b | | | | | | | | |
| K62213621 | dihydroartemisinin | antimalarial agent | RCOR2 | rest corepressor 2 | −0.19911 | 0.00014 | 0.007362 | −0.32446 | M | 0.185453 | M | 0.134506 |
| K62213621 | dihydroartemisinin | antimalarial agent | SALL2 | sal-like protein 2 | −0.18409 | 0.000439 | 0.015554 | −0.32241 | M | 0.185453 | M | 0.134506 |
| K62213621 | dihydroartemisinin | antimalarial agent | SYPL2 | synaptophysin-like protein 2 | −0.20391 | 9.54E−05 | 0.005686 | −0.26159 | M | 0.185453 | M | 0.134506 |
| K64052750 | gefitinib | EGFR inhibitor | ALOX5 | polyunsaturated fatty acid 5- | −0.23253 | 8.53E−06 | 0.00107 | −0.39281 | IM | −0.06642 | IM | −0.05639 |
| | | | | lipoxy genase | | | | | | | | |
| K64052750 | gefitinib | EGFR inhibitor | ANKRD65 | ankyrin repeat domain- | −0.20112 | 0.000125 | 0.006815 | −0.293 | IM | −0.06642 | IM | −0.05639 |
| | | | | containing protein 65 | | | | | | | | |
| K64052750 | gefitinib | EGFR inhibitor | ARHGAP30 | rho gtpase-activating protein 30 | −0.16656 | 0.00154 | 0.034009 | −0.34284 | IM | −0.06642 | IM | −0.05639 |
| K64052750 | gefitinib | EGFR inhibitor | ARHGDIB | rho gdp-dissociation | −0.1575 | 0.002767 | 0.048233 | −0.30282 | IM | −0.06642 | IM | −0.05639 |
| | | | | inhibitor 2 | | | | | | | | |
| K64052750 | gefitinib | EGFR inhibitor | BCL2L14 | apoptosis facilitator bcl-2- | −0.19144 | 0.000264 | 0.011196 | −0.24638 | IM | −0.06642 | IM | −0.05639 |
| | | | | like protein 14 | | | | | | | | |
| K64052750 | gefitinib | EGFR inhibitor | CLEC12A | c-type lectin domain family | −0.20702 | 7.76E−05 | 0.004951 | −0.24566 | IM | −0.06642 | IM | −0.05639 |
| | | | | 12 member a | | | | | | | | |
| K64052750 | gefitinib | EGFR inhibitor | CLEC7A | c-type lectin domain family | −0.24809 | 1.95E−06 | 0.000371 | −0.24173 | IM | −0.06642 | IM | −0.05639 |
| | | | | 7 member a | | | | | | | | |
| K64052750 | gefitinib | EGFR inhibitor | CLIC5 | chloride intracellular | −0.18933 | 0.000309 | 0.012435 | −0.25116 | IM | −0.06642 | IM | −0.05639 |
| | | | | channel protein 5 | | | | | | | | |
| K64052750 | gefitinib | EGFR inhibitor | COL17A1 | collagen alpha-1 | −0.30786 | 2.55E−09 | 2.79E−06 | −0.44664 | IM | −0.06642 | IM | −0.05639 |
| K64052750 | gefitinib | EGFR inhibitor | CXCL16 | c-x-c motif chemokine 16 | −0.26687 | 2.86E−07 | 9.23E−05 | −0.3692 | IM | −0.06642 | IM | −0.05639 |
| K64052750 | gefitinib | EGFR inhibitor | CYP4B1 | cytochrome p450 4b1 | −0.1937 | 0.000222 | 0.010038 | −0.42112 | IM | −0.06642 | IM | −0.05639 |
| K64052750 | gefitinib | EGFR inhibitor | DAPP1 | dual adapter for | −0.30164 | 5.49E−09 | 4.93E−06 | −0.44278 | IM | −0.06642 | IM | −0.05639 |
| | | | | phosphotyrosine and 3- | | | | | | | | |
| | | | | phosphotyrosine and 3- | | | | | | | | |
| | | | | phosphoinositide | | | | | | | | |
| K64052750 | gefitinib | EGFR inhibitor | DENND1C | denn domain-containing | −0.29433 | 1.32E−08 | 9.53E−06 | −0.47596 | IM | −0.06642 | IM | −0.05639 |
| | | | | protein 1c | | | | | | | | |
| K64052750 | gefitinib | EGFR inhibitor | DENND2D | denn domain-containing | −0.21974 | 2.66E−05 | 0.002379 | −0.31211 | IM | −0.06642 | IM | −0.05639 |
| | | | | protein 2d | | | | | | | | |
| K64052750 | gefitinib | EGFR inhibitor | DSG3 | desmoglein-3 | −0.32232 | 4.01E−10 | 6.84E−07 | −0.33991 | IM | −0.06642 | IM | −0.05639 |
| K64052750 | gefitinib | EGFR inhibitor | FGD3 | fyve; rhogef and ph domain- | −0.30658 | 2.99E−09 | 3.13E−06 | −0.42752 | IM | −0.06642 | IM | −0.05639 |
| | | | | containing protein 3 | | | | | | | | |
| K64052750 | gefitinib | EGFR inhibitor | HSH2D | hematopoietic sh2 domain- | −0.16146 | 0.00215 | 0.041575 | −0.41121 | IM | −0.06642 | IM | −0.05639 |
| | | | | containing protein | | | | | | | | |
| K64052750 | gefitinib | EGFR inhibitor | ITGB4 | integrin beta-4 | −0.30107 | 5.88E−09 | 5.19E−06 | −0.48548 | IM | −0.06642 | IM | −0.05639 |
| K64052750 | gefitinib | EGFR inhibitor | KRT14 | keratin; type i cytoskeletal 14 | −0.30795 | 2.52E−09 | 2.77E−06 | −0.29793 | IM | −0.06642 | IM | −0.05639 |
| K64052750 | gefitinib | EGFR inhibitor | KRT16 | keratin; type i cytoskeletal 16 | −0.36143 | 1.61E−12 | 1.17E−08 | −0.42779 | IM | −0.06642 | IM | −0.05639 |
| K64052750 | gefitinib | EGFR inhibitor | KRT17 | keratin; type i cytoskeletal 17 | −0.31231 | 1.46E−09 | 1.85E−06 | −0.38749 | IM | −0.06642 | IM | −0.05639 |
| K64052750 | gefitinib | EGFR inhibitor | KRT5 | keratin; type ii cytoskeletal 5 | −0.31631 | 8.77E−10 | 1.26E−06 | −0.38115 | IM | −0.06642 | IM | −0.05639 |
| K64052750 | gefitinib | EGFR inhibitor | LPAR5 | lysophosphatidic acid | −0.32608 | 2.44E−10 | 4.72E−07 | −0.40871 | IM | −0.06642 | IM | −0.05639 |
| | | | | receptor 5 | | | | | | | | |
| K64052750 | gefitinib | EGFR inhibitor | PTAFR | platelet-activating factor | −0.29641 | 1.03E−08 | 7.91E−06 | −0.49763 | IM | −0.06642 | IM | −0.05639 |
| | | | | receptor | | | | | | | | |
| K64052750 | gefitinib | EGFR inhibitor | PTPN6 | tyrosine-protein phosphatase | −0.35966 | 2.11E−12 | 1.44E−08 | −0.47022 | IM | −0.06642 | IM | −0.05639 |
| | | | | non-receptor type 6 | | | | | | | | |
| K64052750 | gefitinib | EGFR inhibitor | RASGEF1B | ras-gef domain-containing | −0.1924 | 0.000245 | 0.010697 | −0.25038 | IM | −0.06642 | IM | −0.05639 |
| | | | | family member 1b | | | | | | | | |
| K64052750 | gefitinib | EGFR inhibitor | S100A8 | protein s100-a8 | −0.22101 | 2.39E−05 | 0.0022 | −0.32493 | IM | −0.06642 | IM | −0.05639 |
| K64052750 | gefitinib | EGFR inhibitor | SIRPB2 | signal-regulatory protein | −0.18156 | 0.000546 | 0.017889 | −0.29971 | IM | −0.06642 | IM | −0.05639 |
| | | | | beta-2 | | | | | | | | |
| K64052750 | gefitinib | EGFR inhibitor | TEAD3 | transcriptional enhancer | −0.20896 | 6.62E−05 | 0.004445 | −0.29987 | IM | −0.06642 | IM | −0.05639 |
| | | | | factor tef-5 | | | | | | | | |
| K64052750 | gefitinib | EGFR inhibitor | TNFSF10 | tumor necrosis factor ligand | −0.25786 | 7.32E−07 | 0.000183 | −0.28333 | IM | −0.06642 | IM | −0.05639 |
| | | | | superfamily member 10 | | | | | | | | |
| K64052750 | gefitinib | EGFR inhibitor | UNC13D | protein unc-13 homolog d | −0.16568 | 0.001632 | 0.035219 | −0.31337 | IM | −0.06642 | IM | −0.05639 |
| K64052750 | gefitinib | EGFR inhibitor | VIPR1 | vasoactive intestinal | −0.23496 | 6.81E−06 | 0.000909 | −0.30575 | IM | −0.06642 | IM | −0.05639 |
| | | | | polypeptide receptor 1 | | | | | | | | |
| K64052750 | gefitinib | EGFR inhibitor | VSIR | v-type immunoglobulin | −0.19354 | 0.000225 | 0.010113 | −0.29158 | IM | −0.06642 | IM | −0.05639 |
| | | | | domain-containing | | | | | | | | |
| | | | | suppressor of t-cell | | | | | | | | |
| | | | | activation | | | | | | | | |
| K67578145 | GDC-0879 | RAF inhibitor | A2M | alpha-2-macroglobulin | −0.19473 | 0.00028 | 0.011644 | −0.28495 | M | 0.110094 | M | 0.15809 |
| K67578145 | GDC-0879 | RAF inhibitor | APOD | apolipoprotein d | −0.204 | 0.000139 | 0.007331 | −0.31016 | M | 0.110094 | M | 0.15809 |
| K67578145 | GDC-0879 | RAF inhibitor | ASB2 | ankyrin repeat and socs box | −0.18675 | 0.000498 | 0.016875 | −0.35895 | M | 0.110094 | M | 0.15809 |
| | | | | protein 2 | | | | | | | | |
| K67578145 | GDC-0879 | RAF inhibitor | BCL2A1 | bcl-2-related protein a1 | −0.34678 | 3.72E−11 | 1.18E−07 | −0.3663 | M | 0.110094 | M | 0.15809 |
| K67578145 | GDC-0879 | RAF inhibitor | C3orf70 | upf0524 protein c3orf70 | −0.21119 | 7.9E−05 | 0.005012 | −0.31553 | M | 0.110094 | M | 0.15809 |
| K67578145 | GDC-0879 | RAF inhibitor | CD96 | t-cell surface protein tactile | −0.27983 | 1.31E−07 | 5.2E−05 | −0.33572 | M | 0.110094 | M | 0.15809 |
| K67578145 | GDC-0879 | RAF inhibitor | CDH19 | cadherin-19 | −0.32149 | 1.04E−09 | 1.42E−06 | −0.33921 | M | 0.110094 | M | 0.15809 |
| K67578145 | GDC-0879 | RAF inhibitor | CHL1 | atp-dependent dna helicase | −0.23897 | 7.42E−06 | 0.000969 | −0.2718 | M | 0.110094 | M | 0.15809 |
| | | | | ddx11-related | | | | | | | | |
| K67578145 | GDC-0879 | RAF inhibitor | CMTM5 | cklf-like marvel | −0.19123 | 0.000361 | 0.01374 | −0.25048 | M | 0.110094 | M | 0.15809 |
| | | | | transmembrane domain- | | | | | | | | |
| | | | | containing protein 5 | | | | | | | | |
| K67578145 | GDC-0879 | RAF inhibitor | COL19A1 | collagen alpha-1 | −0.34368 | 5.68E−11 | 1.57E−07 | −0.37532 | M | 0.110094 | M | 0.15809 |
| K67578145 | GDC-0879 | RAF inhibitor | COL9A3 | collagen alpha-3 | −0.24562 | 4.03E−06 | 0.000623 | −0.33425 | M | 0.110094 | M | 0.15809 |
| K67578145 | GDC-0879 | RAF inhibitor | CSPG4 | chondroitin sulfate | −0.17113 | 0.001442 | 0.032684 | −0.27829 | M | 0.110094 | M | 0.15809 |
| | | | | proteoglycan 4 | | | | | | | | |
| K67578145 | GDC-0879 | RAF inhibitor | CTLA4 | cytotoxic t-lymphocyte | −0.22352 | 2.87E−05 | 0.002503 | −0.26156 | M | 0.110094 | M | 0.15809 |
| | | | | protein 4 | | | | | | | | |
| K67578145 | GDC-0879 | RAF inhibitor | CUBN | cubilin | −0.21472 | 5.95E−05 | 0.00413 | −0.30043 | M | 0.110094 | M | 0.15809 |
| K67578145 | GDC-0879 | RAF inhibitor | DAAM2 | disheveled-associated | −0.24419 | 4.6E−06 | 0.000685 | −0.33766 | M | 0.110094 | M | 0.15809 |
| | | | | activator of morphogenesis 2 | | | | | | | | |
| K67578145 | GDC-0879 | RAF inhibitor | EDNRB | endothelin receptor type b | −0.31921 | 1.38E−09 | 1.77E−06 | −0.33396 | M | 0.110094 | M | 0.15809 |
| K67578145 | GDC-0879 | RAF inhibitor | FAM180B | protein fam180b | −0.25254 | 2.09E−06 | 0.00039 | −0.27608 | M | 0.110094 | M | 0.15809 |
| K67578145 | GDC-0879 | RAF inhibitor | FCGR2A | low affinity | −0.23701 | 8.86E−06 | 0.001102 | −0.31278 | M | 0.110094 | M | 0.15809 |
| | | | | immunoglobulin gamma fc | | | | | | | | |
| | | | | region receptor ii-a-related | | | | | | | | |
| K67578145 | GDC-0879 | RAF inhibitor | FCGR2B | low affinity | −0.28835 | 5.19E−08 | 2.6E−05 | −0.34392 | M | 0.110094 | M | 0.15809 |
| | | | | immunoglobulin gamma fc | | | | | | | | |
| | | | | region receptor ii-b | | | | | | | | |
| K67578145 | GDC-0879 | RAF inhibitor | FCMR | fas apoptotic inhibitory | −0.18429 | 0.000592 | 0.01879 | −0.25878 | M | 0.110094 | M | 0.15809 |
| | | | | molecule 3 | | | | | | | | |
| K67578145 | GDC-0879 | RAF inhibitor | FCRLA | fc receptor-like a | −0.31559 | 2.16E−09 | 2.45E−06 | −0.393 | M | 0.110094 | M | 0.15809 |
| K67578145 | GDC-0879 | RAF inhibitor | FLT1 | vascular endothelial growth | −0.32593 | 5.9E−10 | 9.16E−07 | −0.32132 | M | 0.110094 | M | 0.15809 |
| | | | | factor receptor 1 | | | | | | | | |
| K67578145 | GDC-0879 | RAF inhibitor | GAS7 | growth arrest-specific | −0.28738 | 5.77E−08 | 2.81E−05 | −0.39355 | M | 0.110094 | M | 0.15809 |
| | | | | protein 7 | | | | | | | | |
| K67578145 | GDC-0879 | RAF inhibitor | GPR55 | g-protein coupled receptor 55 | −0.24154 | 5.88E−06 | 0.000815 | −0.31117 | M | 0.110094 | M | 0.15809 |
| K67578145 | GDC-0879 | RAF inhibitor | HPS5 | hermansky-pudlak | −0.17848 | 0.000884 | 0.024189 | −0.25407 | M | 0.110094 | M | 0.15809 |
| | | | | syndrome 5 protein | | | | | | | | |
| K67578145 | GDC-0879 | RAF inhibitor | IL12RB2 | interleukin-12 receptor | −0.25067 | 2.51E−06 | 0.000443 | −0.2562 | M | 0.110094 | M | 0.15809 |
| | | | | subunit beta-2 | | | | | | | | |
| K67578145 | GDC-0879 | RAF inhibitor | IL16 | pro-interleukin-16 | −0.30208 | 1.09E−08 | 8.22E−06 | −0.44471 | M | 0.110094 | M | 0.15809 |
| K67578145 | GDC-0879 | RAF inhibitor | IRF4 | interferon regulatory factor 4 | −0.29742 | 1.87E−08 | 1.23E−05 | −0.40019 | M | 0.110094 | M | 0.15809 |
| K67578145 | GDC-0879 | RAF inhibitor | ITGA9 | integrin alpha-9 | −0.16361 | 0.002334 | 0.043621 | −0.29985 | M | 0.110094 | M | 0.15809 |
| K67578145 | GDC-0879 | RAF inhibitor | KCNJ10 | atp-sensitive inward rectifier | −0.24289 | 5.19E−06 | 0.000746 | −0.27857 | M | 0.110094 | M | 0.15809 |
| | | | | potassium channel 10 | | | | | | | | |
| K67578145 | GDC-0879 | RAF inhibitor | MIA | melanoma-derived growth | −0.30481 | 7.9E−09 | 6.51E−06 | −0.40986 | M | 0.110094 | M | 0.15809 |
| | | | | regulatory protein | | | | | | | | |
| K67578145 | GDC-0879 | RAF inhibitor | NFATC2 | nuclear factor of activated t- | −0.28788 | 5.47E−08 | 2.7E−05 | −0.32127 | M | 0.110094 | M | 0.15809 |
| | | | | cells; cytoplasmic 2 | | | | | | | | |
| K67578145 | GDC-0879 | RAF inhibitor | NGFR | tumor necrosis factor | −0.17125 | 0.001431 | 0.032537 | −0.34907 | M | 0.110094 | M | 0.15809 |
| | | | | receptor superfamily | | | | | | | | |
| | | | | member 16 | | | | | | | | |
| K67578145 | GDC-0879 | RAF inhibitor | NRROS | transforming growth factor | −0.27944 | 1.37E−07 | 5.34E−05 | −0.28505 | M | 0.110094 | M | 0.15809 |
| | | | | beta activator lrrc33 | | | | | | | | |
| K67578145 | GDC-0879 | RAF inhibitor | P2RX7 | p2x purinoceptor 7 | −0.16681 | 0.001907 | 0.038637 | −0.25968 | M | 0.110094 | M | 0.15809 |
| K67578145 | GDC-0879 | RAF inhibitor | PKNOX2 | homeobox protein pknox2 | −0.2358 | 9.87E−06 | 0.001188 | −0.31192 | M | 0.110094 | M | 0.15809 |
| K67578145 | GDC-0879 | RAF inhibitor | PLEKHO2 | pleckstrin homology | −0.19019 | 0.00039 | 0.014408 | −0.24616 | M | 0.110094 | M | 0.15809 |
| | | | | domain-containing family o | | | | | | | | |
| | | | | member 2 | | | | | | | | |
| K67578145 | GDC-0879 | RAF inhibitor | PLP1 | myelin proteolipid protein | −0.32079 | 1.13E−09 | 1.52E−06 | −0.38941 | M | 0.110094 | M | 0.15809 |
| K67578145 | GDC-0879 | RAF inhibitor | PMP2 | myelin p2 protein | −0.34413 | 5.35E−11 | 1.51E−07 | −0.25593 | M | 0.110094 | M | 0.15809 |
| K67578145 | GDC-0879 | RAF inhibitor | PTCRA | pre t-cell antigen receptor | −0.28745 | 5.73E−08 | 2.79E−05 | −0.35555 | M | 0.110094 | M | 0.15809 |
| | | | | alpha | | | | | | | | |
| K67578145 | GDC-0879 | RAF inhibitor | PTPRZ1 | receptor-type tyrosine- | −0.18312 | 0.000643 | 0.019793 | −0.34813 | M | 0.110094 | M | 0.15809 |
| | | | | protein phosphatase zeta | | | | | | | | |
| K67578145 | GDC-0879 | RAF inhibitor | RENBP | n-acylglucosamine 2- | −0.21597 | 5.37E−05 | 0.003856 | −0.28553 | M | 0.110094 | M | 0.15809 |
| | | | | epimerase | | | | | | | | |
| K67578145 | GDC-0879 | RAF inhibitor | RHOJ | rho-related gtp-binding | −0.26738 | 4.83E−07 | 0.000135 | −0.29934 | M | 0.110094 | M | 0.15809 |
| | | | | protein rhoj | | | | | | | | |
| K67578145 | GDC-0879 | RAF inhibitor | SCML4 | sex comb on midleg-like | −0.28099 | 1.16E−07 | 4.75E−05 | −0.32686 | M | 0.110094 | M | 0.15809 |
| | | | | protein 4 | | | | | | | | |
| K67578145 | GDC-0879 | RAF inhibitor | SHC4 | shc-transforming protein 4 | −0.25967 | 1.05E−06 | 0.000237 | −0.27344 | M | 0.110094 | M | 0.15809 |
| K67578145 | GDC-0879 | RAF inhibitor | SHROOM4 | protein shroom4 | −0.19967 | 0.000193 | 0.009147 | −0.25423 | M | 0.110094 | M | 0.15809 |
| K67578145 | GDC-0879 | RAF inhibitor | SLC35F1 | solute carrier family 35 | −0.24073 | 6.32E−06 | 0.000862 | −0.24517 | M | 0.110094 | M | 0.15809 |
| | | | | member f1 | | | | | | | | |
| K67578145 | GDC-0879 | RAF inhibitor | SORCS1 | vps10 domain-containing | −0.29938 | 1.49E−08 | 1.04E−05 | −0.27276 | M | 0.110094 | M | 0.15809 |
| | | | | receptor sorcs1 | | | | | | | | |
| K67578145 | GDC-0879 | RAF inhibitor | SOX5 | transcription factor sox-5 | −0.20918 | 9.27E−05 | 0.005571 | −0.29398 | M | 0.110094 | M | 0.15809 |
| K67578145 | GDC-0879 | RAF inhibitor | ST6GALNAC3 | alpha-n- | −0.18297 | 0.00065 | 0.019928 | −0.26558 | M | 0.110094 | M | 0.15809 |
| | | | | acetylgalactosaminide | | | | | | | | |
| | | | | alpha-2; 6-sialyltransferase 3 | | | | | | | | |
| K67578145 | GDC-0879 | RAF inhibitor | ST8SIA1 | alpha-n-acetylneuraminide | −0.25962 | 1.05E−06 | 0.000238 | −0.26401 | M | 0.110094 | M | 0.15809 |
| | | | | alpha-2; 8-sialy ltransferase | | | | | | | | |
| K67578145 | GDC-0879 | RAF inhibitor | TAMALIN | protein tamalin | −0.22305 | 2.98E−05 | 0.002576 | −0.24803 | M | 0.110094 | M | 0.15809 |
| K67578145 | GDC-0879 | RAF inhibitor | TNFRSF14 | tumor necrosis factor | −0.2291 | 1.78E−05 | 0.001799 | −0.32768 | M | 0.110094 | M | 0.15809 |
| | | | | receptor superfamily | | | | | | | | |
| | | | | member 14 | | | | | | | | |
| K67578145 | GDC-0879 | RAF inhibitor | TRPV2 | transient receptor potential | −0.26514 | 6.06E−07 | 0.00016 | −0.31001 | M | 0.110094 | M | 0.15809 |
| | | | | cation channel subfamily v | | | | | | | | |
| | | | | member 2 | | | | | | | | |
| K69726342 | atorvastatin | HMGCR inhibitor | ANGPTL1 | angiopoietin-related protein 1 | −0.17346 | 0.000833 | 0.023275 | −0.25955 | M | 0.115334 | M | 0.076984 |
| K69726342 | atorvastatin | HMGCR inhibitor | ANTXR1 | anthrax toxin receptor 1 | −0.17036 | 0.001034 | 0.026712 | −0.27092 | M | 0.115334 | M | 0.076984 |
| K69726342 | atorvastatin | HMGCR inhibitor | ARMCX1 | armadillo repeat-containing | −0.21491 | 3.22E−05 | 0.002713 | −0.2517 | M | 0.115334 | M | 0.076984 |
| | | | | x-linked protein 1 | | | | | | | | |
| K69726342 | atorvastatin | HMGCR inhibitor | CHST10 | carbohydrate | −0.18388 | 0.000391 | 0.014446 | −0.27109 | M | 0.115334 | M | 0.076984 |
| | | | | sulfotransferase 10 | | | | | | | | |
| K69726342 | atorvastatin | HMGCR inhibitor | CSPG4 | chondroitin sulfate | −0.18244 | 0.000436 | 0.015485 | −0.28967 | M | 0.115334 | M | 0.076984 |
| | | | | proteoglycan 4 | | | | | | | | |
| K69726342 | atorvastatin | HMGCR inhibitor | EPHA3 | ephrin type-a receptor 3 | −0.23994 | 3.23E−06 | 0.000533 | −0.26891 | M | 0.115334 | M | 0.076984 |
| K69726342 | atorvastatin | HMGCR inhibitor | EVC | ellis-van creveld syndrome | −0.20553 | 7.13E−05 | 0.004684 | −0.2736 | M | 0.115334 | M | 0.076984 |
| | | | | protein | | | | | | | | |
| K69726342 | atorvastatin | HMGCR inhibitor | EXTL2 | exostosin-2-related | −0.17388 | 0.000809 | 0.022863 | −0.2948 | M | 0.115334 | M | 0.076984 |
| K69726342 | atorvastatin | HMGCR inhibitor | FYN | tyrosine-protein kinase fyn | −0.15868 | 0.002265 | 0.042866 | −0.26402 | M | 0.115334 | M | 0.076984 |
| K69726342 | atorvastatin | HMGCR inhibitor | IFFO1 | intermediate filament family | −0.19621 | 0.000152 | 0.007782 | −0.25507 | M | 0.115334 | M | 0.076984 |
| | | | | orphan 1 | | | | | | | | |
| K69726342 | atorvastatin | HMGCR inhibitor | LPAR4 | lysophosphatidic acid | −0.20112 | 0.000102 | 0.005959 | −0.28366 | M | 0.115334 | M | 0.076984 |
| | | | | receptor 4 | | | | | | | | |
| K69726342 | atorvastatin | HMGCR inhibitor | MAP3K3 | mitogen-activated protein | −0.18539 | 0.00035 | 0.01346 | −0.24978 | M | 0.115334 | M | 0.076984 |
| | | | | kinase kinase kinase 3 | | | | | | | | |
| K69726342 | atorvastatin | HMGCR inhibitor | MOXD1 | dbh-like monooxygenase | −0.20311 | 8.71E−05 | 0.005343 | −0.2464 | M | 0.115334 | M | 0.076984 |
| | | | | protein 1 | | | | | | | | |
| K69726342 | atorvastatin | HMGCR inhibitor | PDE1C | calcium/calmodulin- | −0.22212 | 1.71E−05 | 0.00175 | −0.26836 | M | 0.115334 | M | 0.076984 |
| | | | | dependent 3′; 5′-cyclic | | | | | | | | |
| | | | | nucleotide | | | | | | | | |
| | | | | phosphodiesterase 1c | | | | | | | | |
| K69726342 | atorvastatin | HMGCR inhibitor | RASSF8 | ras association domain- | −0.16853 | 0.001173 | 0.02884 | −0.28561 | M | 0.115334 | M | 0.076984 |
| | | | | containing protein 8 | | | | | | | | |
| K69726342 | atorvastatin | HMGCR inhibitor | RHOJ | rho-related gtp-binding | −0.20892 | 5.37E−05 | 0.003856 | −0.34189 | M | 0.115334 | M | 0.076984 |
| | | | | protein rhoj | | | | | | | | |
| K69726342 | atorvastatin | HMGCR inhibitor | SLC35F1 | solute carrier family 35 | −0.16358 | 0.001641 | 0.035325 | −0.26581 | M | 0.115334 | M | 0.076984 |
| | | | | member f1 | | | | | | | | |
| K69726342 | atorvastatin | HMGCR inhibitor | SPARC | sparc | −0.19929 | 0.000119 | 0.006594 | −0.25485 | M | 0.115334 | M | 0.076984 |
| K69726342 | atorvastatin | HMGCR inhibitor | TIMP2 | metalloproteinase inhibitor 2 | −0.1812 | 0.000477 | 0.016423 | −0.23894 | M | 0.115334 | M | 0.076984 |
| K69726342 | atorvastatin | HMGCR inhibitor | VIM | vimentin | −0.18855 | 0.000275 | 0.01153 | −0.24243 | M | 0.115334 | M | 0.076984 |
| K69726342 | atorvastatin | HMGCR inhibitor | ZCCHC24 | | −0.23137 | 7.31E−06 | 0.000958 | −0.24867 | M | 0.115334 | M | 0.076984 |
| K70914287 | BIBX-1382 | EGFR inhibitor | ALOX5 | polyunsaturated fatty acid 5- | −0.18571 | 0.000341 | 0.013253 | −0.35824 | IM | −0.03277 | IM | −0.07259 |
| | | | | lipoxygenase | | | | | | | | |
| K70914287 | BIBX-1382 | EGFR inhibitor | COL17A1 | collagen alpha-1 | −0.29556 | 7.47E−09 | 6.23E−06 | −0.35198 | IM | −0.03277 | IM | −0.07259 |
| K70914287 | BIBX-1382 | EGFR inhibitor | CXCL16 | c-x-c motif chemokine 16 | −0.2049 | 7.51E−05 | 0.004846 | −0.36142 | IM | −0.03277 | IM | −0.07259 |
| K70914287 | BIBX-1382 | EGFR inhibitor | DAPP1 | dual adapter for | −0.30397 | 2.64E−09 | 2.85E−06 | −0.35553 | IM | −0.03277 | IM | −0.07259 |
| | | | | phosphotyrosine and 3- | | | | | | | | |
| | | | | phosphotyrosine and 3- | | | | | | | | |
| | | | | phosphoinositide | | | | | | | | |
| K70914287 | BIBX-1382 | EGFR inhibitor | DENND1C | denn domain-containing | −0.24952 | 1.25E−06 | 0.00027 | −0.36569 | IM | −0.03277 | IM | −0.07259 |
| | | | | protein 1c | | | | | | | | |
| K70914287 | BIBX-1382 | EGFR inhibitor | DENND2D | denn domain-containing | −0.20548 | 7.16E−05 | 0.004698 | −0.32721 | IM | −0.03277 | IM | −0.07259 |
| | | | | protein 2d | | | | | | | | |
| K70914287 | BIBX-1382 | EGFR inhibitor | DSG3 | desmoglein-3 | −0.32907 | 9.65E−11 | 2.36E−07 | −0.34731 | IM | −0.03277 | IM | −0.07259 |
| K70914287 | BIBX-1382 | EGFR inhibitor | FGD3 | fyve; rhogef and ph domain- | −0.23794 | 3.92E−06 | 0.000612 | −0.26577 | IM | −0.03277 | IM | −0.07259 |
| | | | | containing protein 3 | | | | | | | | |
| K70914287 | BIBX-1382 | EGFR inhibitor | ITGB4 | integrin beta-4 | −0.30844 | 1.5E−09 | 1.87E−06 | −0.43325 | IM | −0.03277 | IM | −0.07259 |
| K70914287 | BIBX-1382 | EGFR inhibitor | KRT16 | keratin; type i cytoskeletal 16 | −0.29358 | 9.49E−09 | 7.45E−06 | −0.31999 | IM | −0.03277 | IM | −0.07259 |
| K70914287 | BIBX-1382 | EGFR inhibitor | KRT17 | keratin; type i cytoskeletal 17 | −0.32442 | 1.82E−10 | 3.83E−07 | −0.27453 | IM | −0.03277 | IM | −0.07259 |
| K70914287 | BIBX-1382 | EGFR inhibitor | KRT5 | keratin; type ii cytoskeletal 5 | −0.33811 | 2.72E−11 | 9.34E−08 | −0.26105 | IM | −0.03277 | IM | −0.07259 |
| K70914287 | BIBX-1382 | EGFR inhibitor | LPAR5 | lysophosphatidic acid | −0.30791 | 1.6E−09 | 1.95E−06 | −0.28773 | IM | −0.03277 | IM | −0.07259 |
| | | | | receptor 5 | | | | | | | | |
| K70914287 | BIBX-1382 | EGFR inhibitor | PTAFR | platelet-activating factor | −0.30869 | 1.45E−09 | 1.84E−06 | −0.39581 | IM | −0.03277 | IM | −0.07259 |
| | | | | receptor | | | | | | | | |
| K70914287 | BIBX-1382 | EGFR inhibitor | PTPN6 | tyrosine-protein phosphatase | −0.29938 | 4.67E−09 | 4.37E−06 | −0.35933 | IM | −0.03277 | IM | −0.07259 |
| | | | | non-receptor type 6 | | | | | | | | |
| K70914287 | BIBX-1382 | EGFR inhibitor | S100A8 | protein s100-a8 | −0.20435 | 7.86E−05 | 0.004997 | −0.24681 | IM | −0.03277 | IM | −0.07259 |
| K70914287 | BIBX-1382 | EGFR inhibitor | SH3BP1 | bargin-related | −0.21721 | 2.64E−05 | 0.002361 | −0.24431 | IM | −0.03277 | IM | −0.07259 |
| K70914287 | BIBX-1382 | EGFR inhibitor | SIRPB2 | signal-regulatory protein | −0.24529 | 1.91E−06 | 0.000366 | −0.25676 | IM | −0.03277 | IM | −0.07259 |
| | | | | beta-2 | | | | | | | | |
| K70914287 | BIBX-1382 | EGFR inhibitor | TNFSF10 | tumor necrosis factor ligand | −0.23944 | 3.39E−06 | 0.000553 | −0.2628 | IM | −0.03277 | IM | −0.07259 |
| | | | | superfamily member 10 | | | | | | | | |
| K70914287 | BIBX-1382 | EGFR inhibitor | VSIR | v-type immunoglobulin | −0.18516 | 0.000356 | 0.013612 | −0.25087 | IM | −0.03277 | IM | −0.07259 |
| | | | | domain-containing | | | | | | | | |
| | | | | suppressor of t-cell | | | | | | | | |
| | | | | activation | | | | | | | | |
| K70914287 | BIBX-1382 | EGFR inhibitor | ZMYND15 | zinc finger mynd domain- | −0.16085 | 0.001966 | 0.039377 | −0.31983 | IM | −0.03277 | IM | −0.07259 |
| | | | | containing protein 15 | | | | | | | | |
| K72723676 | benzethonium | sodium channel blocker | COL19A1 | collagen alpha-1 | −0.19717 | 0.000156 | 0.007922 | −0.25833 | M | 0.156786 | M | 0.160433 |
| K72723676 | benzethonium | sodium channel blocker | COL9A3 | collagen alpha-3 | −0.1639 | 0.00173 | 0.036494 | −0.29636 | M | 0.156786 | M | 0.160433 |
| K72723676 | benzethonium | sodium channel blocker | IL12RB2 | interleukin-12 receptor | −0.25035 | 1.36E−06 | 0.000286 | −0.26523 | M | 0.156786 | M | 0.160433 |
| | | | | subunit beta-2 | | | | | | | | |
| K72723676 | benzethonium | sodium channel blocker | MIA | melanoma-derived growth | −0.17016 | 0.001136 | 0.028321 | −0.2637 | M | 0.156786 | M | 0.160433 |
| | | | | regulatory protein | | | | | | | | |
| K72723676 | benzethonium | sodium channel blocker | SGCD | delta-sarcoglycan | −0.16427 | 0.001687 | 0.035964 | −0.24047 | M | 0.156786 | M | 0.160433 |
| K72723676 | benzethonium | sodium channel blocker | TMEM229B | transmembrane protein 229b | −0.19379 | 0.000203 | 0.009462 | −0.23742 | M | 0.156786 | M | 0.160433 |
| K76239644 | BMS-690514 | EGFR inhibitor; | ALOX5 | polyunsaturated fatty acid 5- | −0.22225 | 1.78E−05 | 0.001798 | −0.27378 | IM | −0.16658 | IM | 0.013462 |
| | | VEGFR inhibitor | | lipoxygenase | | | | | | | | |
| K76239644 | BMS-690514 | EGFR inhibitor; | ANKRD65 | ankyrin repeat domain- | −0.17958 | 0.000557 | 0.018084 | −0.25755 | IM | −0.16658 | IM | 0.013462 |
| | | VEGFR inhibitor | | containing protein 65 | | | | | | | | |
| K76239644 | BMS-690514 | EGFR inhibitor; | CMTM4 | cklf-like marvel | −0.15962 | 0.002192 | 0.042061 | −0.29029 | IM | −0.16658 | IM | 0.013462 |
| | | VEGFR inhibitor | | transmembrane domain- | | | | | | | | |
| | | | | containing protein 4 | | | | | | | | |
| K76239644 | BMS-690514 | EGFR inhibitor; | COL17A1 | collagen alpha-1 | −0.28881 | 1.84E−08 | 1.21E−05 | −0.28845 | IM | −0.16658 | IM | 0.013462 |
| | | VEGFR inhibitor | | | | | | | | | | |
| K76239644 | BMS-690514 | EGFR inhibitor; | CXCL16 | c-x-c motif chemokine 16 | −0.27048 | 1.48E−07 | 5.67E−05 | −0.30335 | IM | −0.16658 | IM | 0.013462 |
| | | VEGFR inhibitor | | | | | | | | | | |
| K76239644 | BMS-690514 | EGFR inhibitor; | DAPP1 | dual adapter for | −0.33361 | 5.79E−11 | 1.59E−07 | −0.36829 | IM | −0.16658 | IM | 0.013462 |
| | | VEGFR inhibitor | | phosphotyrosine and 3- | | | | | | | | |
| | | | | phosphotyrosine and 3- | | | | | | | | |
| | | | | phosphoinositide | | | | | | | | |
| K76239644 | BMS-690514 | EGFR inhibitor; | DENNDIC | denn domain-containing | −0.31007 | 1.35E−09 | 1.74E−06 | −0.38827 | IM | −0.16658 | IM | 0.013462 |
| | | VEGFR inhibitor | | protein 1c | | | | | | | | |
| K76239644 | BMS-690514 | EGFR inhibitor; | DSG3 | desmoglein-3 | −0.31239 | 1E−09 | 1.39E−06 | −0.30488 | IM | −0.16658 | IM | 0.013462 |
| | | VEGFR inhibitor | | | | | | | | | | |
| K76239644 | BMS-690514 | EGFR inhibitor; | FGD3 | fyve; rhogef and ph domain- | −0.24179 | 2.87E−06 | 0.000489 | −0.39 | IM | −0.16658 | IM | 0.013462 |
| | | VEGFR inhibitor | | containing protein 3 | | | | | | | | |
| K76239644 | BMS-690514 | EGFR inhibitor; | HSH2D | hematopoietic sh2 domain- | −0.24558 | 1.98E−06 | 0.000375 | −0.2877 | IM | −0.16658 | IM | 0.013462 |
| | | VEGFR inhibitor | | containing protein | | | | | | | | |
| K76239644 | BMS-690514 | EGFR inhibitor; | IL 15RA | interleukin-15 receptor | −0.22831 | 1.03E−05 | 0.001223 | −0.24731 | IM | −0.16658 | IM | 0.013462 |
| | | VEGFR inhibitor | | subunit alpha | | | | | | | | |
| K76239644 | BMS-690514 | EGFR inhibitor; | ITGB4 | integrin beta-4 | −0.39573 | 3.59E−15 | 1.41E−10 | −0.37556 | IM | −0.16658 | IM | 0.013462 |
| | | VEGFR inhibitor | | | | | | | | | | |
| K76239644 | BMS-690514 | EGFR inhibitor; | KLF8 | krueppel-like factor 8 | −0.18393 | 0.000405 | 0.014761 | −0.24754 | IM | −0.16658 | IM | 0.013462 |
| | | VEGFR inhibitor | | | | | | | | | | |
| K76239644 | BMS-690514 | EGFR inhibitor; | KRT16 | keratin; type i cytoskeletal | −0.34893 | 6.43E−12 | 3.13E−08 | −0.3908 | IM | −0.16658 | IM | 0.013462 |
| | | VEGFR inhibitor | | 16 | | | | | | | | |
| K76239644 | BMS-690514 | EGFR inhibitor; | KRT17 | keratin; type i cytoskeletal | −0.28716 | 2.23E−08 | 1.39E−05 | −0.29638 | IM | −0.16658 | IM | 0.013462 |
| | | VEGFR inhibitor | | 17 | | | | | | | | |
| K76239644 | BMS-690514 | EGFR inhibitor; | KRT5 | keratin; type ii cytoskeletal | −0.28853 | 1.9E−08 | 1.24E−05 | −0.3001 | IM | −0.16658 | IM | 0.013462 |
| | | VEGFR inhibitor | | 5 | | | | | | | | |
| K76239644 | BMS-690514 | EGFR inhibitor; | LPAR5 | lysophosphatidic acid | −0.30003 | 4.75E−09 | 4.41E−06 | −0.25711 | IM | −0.16658 | IM | 0.013462 |
| | | VEGFR inhibitor | | receptor 5 | | | | | | | | |
| K76239644 | BMS-690514 | EGFR inhibitor; | LTB | lymphotoxin-beta | −0.20853 | 5.82E−05 | 0.00407 | −0.24231 | IM | −0.16658 | IM | 0.013462 |
| | | VEGFR inhibitor | | | | | | | | | | |
| K76239644 | BMS-690514 | EGFR inhibitor; | MAPK10 | mitogen-activated protein | −0.23304 | 6.63E−06 | 0.000892 | −0.26455 | IM | −0.16658 | IM | 0.013462 |
| | | VEGFR inhibitor | | kinase 10 | | | | | | | | |
| K76239644 | BMS-690514 | EGFR inhibitor; | NTN4 | netrin-4 | −0.23336 | 6.43E−06 | 0.000873 | −0.25006 | IM | −0.16658 | IM | 0.013462 |
| | | VEGFR inhibitor | | | | | | | | | | |
| K76239644 | BMS-690514 | EGFR inhibitor; | PSME1 | proteasome activator | −0.16232 | 0.001837 | 0.03784 | −0.24677 | IM | −0.16658 | IM | 0.013462 |
| | | VEGFR inhibitor | | complex subunit 1 | | | | | | | | |
| K76239644 | BMS-690514 | EGFR inhibitor; | PTAFR | platelet-activating factor | −0.31838 | 4.57E−10 | 7.54E−07 | −0.39812 | IM | −0.16658 | IM | 0.013462 |
| | | VEGFR inhibitor | | receptor | | | | | | | | |
| K76239644 | BMS-690514 | EGFR inhibitor; | PTPN6 | tyrosine-protein phosphatase | −0.38357 | 2.83E−14 | 7.28E−10 | −0.38863 | IM | −0.16658 | IM | 0.013462 |
| | | VEGFR inhibitor | | non-receptor type 6 | | | | | | | | |
| K76239644 | BMS-690514 | EGFR inhibitor; | SPTLC2 | serine palmitoyltransferase 2 | −0.16578 | 0.001458 | 0.032905 | −0.25912 | IM | −0.16658 | IM | 0.013462 |
| | | VEGFR inhibitor | | | | | | | | | | |
| K76239644 | BMS-690514 | EGFR inhibitor; | TEAD3 | transcriptional enhancer | −0.1984 | 0.000133 | 0.007116 | −0.24584 | IM | −0.16658 | IM | 0.013462 |
| | | VEGFR inhibitor | | factor tef-5 | | | | | | | | |
| K76239644 | BMS-690514 | EGFR inhibitor; | TNFSF10 | tumor necrosis factor ligand | −0.3578 | 1.71E−12 | 1.22E−08 | −0.2503 | IM | −0.16658 | IM | 0.013462 |
| | | VEGFR inhibitor | | superfamily member 10 | | | | | | | | |
| K76239644 | BMS-690514 | EGFR inhibitor; | UNC13D | protein unc-13 homolog d | −0.20426 | 8.29E−05 | 0.005171 | −0.32715 | IM | −0.16658 | IM | 0.013462 |
| | | VEGFR inhibitor | | | | | | | | | | |
| K76239644 | BMS-690514 | EGFR inhibitor; | VAV1 | proto-oncogene vav | −0.20709 | 6.56E−05 | 0.004423 | −0.24498 | IM | −0.16658 | IM | 0.013462 |
| | | VEGFR inhibitor | | | | | | | | | | |
| K76239644 | BMS-690514 | EGFR inhibitor; | VSIR | v-type immunoglobulin | −0.20258 | 9.51E−05 | 0.005674 | −0.25307 | IM | −0.16658 | IM | 0.013462 |
| | | VEGFR inhibitor | | domain-containing | | | | | | | | |
| | | | | suppressor of t-cell | | | | | | | | |
| | | | | activation | | | | | | | | |
| K78096648 | oxiperomide | dopamine receptor | ADAM11 | disintegrin and | −0.19003 | 0.000293 | 0.012012 | −0.26832 | M | 0.16608 | MSL/M | 0.085408 |
| | | antagonist | | metalloproteinase domain- | | | | | | | | |
| | | | | containing protein 11 | | | | | | | | |
| K78096648 | oxiperomide | dopamine receptor | DVL2 | segment polarity protein | −0.17028 | 0.0012 | 0.029232 | −0.25866 | M | 0.16608 | MSL/M | 0.085408 |
| | | antagonist | | dishevelled homolog dvl-2 | | | | | | | | |
| K78096648 | oxiperomide | dopamine receptor | KBTBD6 | kelch repeat and btb | −0.16844 | 0.001359 | 0.031527 | −0.23969 | M | 0.16608 | MSL/M | 0.085408 |
| | | antagonist | | domain-containing protein 6 | | | | | | | | |
| K78096648 | oxiperomide | dopamine receptor | KCNJ4 | inward rectifier potassium | −0.15711 | 0.002836 | 0.048942 | −0.25506 | M | 0.16608 | MSL/M | 0.085408 |
| | | antagonist | | channel 4 | | | | | | | | |
| K78096648 | oxiperomide | dopamine receptor | MLLT1 | protein enl | −0.16583 | 0.001616 | 0.035009 | −0.32063 | M | 0.16608 | MSL/M | 0.085408 |
| | | antagonist | | | | | | | | | | |
| K78096648 | oxiperomide | dopamine receptor | ZNF101 | zinc finger protein 101 | −0.15976 | 0.002397 | 0.044342 | −0.26227 | M | 0.16608 | MSL/M | 0.085408 |
| | | antagonist | | | | | | | | | | |
| K80343549 | TAK-285 | EGFR inhibitor | DENND1C | denn domain-containing | −0.16238 | 0.002243 | 0.042614 | −0.26121 | IM | −0.07158 | IM | −0.06872 |
| | | | | protein 1c | | | | | | | | |
| K80343549 | TAK-285 | EGFR inhibitor | DENND2D | denn domain-containing | −0.16242 | 0.002238 | 0.042562 | −0.27192 | IM | −0.07158 | IM | −0.06872 |
| | | | | protein 2d | | | | | | | | |
| K80343549 | TAK-285 | EGFR inhibitor | ITGB4 | integrin beta-4 | −0.17623 | 0.000898 | 0.024425 | −0.3462 | IM | −0.07158 | IM | −0.06872 |
| K80343549 | TAK-285 | EGFR inhibitor | LPAR5 | lysophosphatidic acid | −0.22432 | 2.16E−05 | 0.002054 | −0.27437 | IM | −0.07158 | IM | −0.06872 |
| | | | | receptor 5 | | | | | | | | |
| K93123848 | RAF265 | RAF inhibitor; VEGFR | ADAM23 | disintegrin and | −0.19822 | 0.000129 | 0.006981 | −0.2473 | M | 0.101093 | M | 0.191571 |
| | | inhibitor | | metalloproteinase domain- | | | | | | | | |
| | | | | containing protein 23 | | | | | | | | |
| K93123848 | RAF265 | RAF inhibitor; VEGFR | AEBP1 | adipocyte enhancer-binding | −0.16893 | 0.001142 | 0.028405 | −0.24081 | M | 0.101093 | M | 0.191571 |
| | | inhibitor | | protein 1 | | | | | | | | |
| K93123848 | RAF265 | RAF inhibitor; VEGFR | ARHGAP31 | rho gtpase-activating protein | −0.21747 | 2.58E−05 | 0.002321 | −0.24902 | M | 0.101093 | M | 0.191571 |
| | | inhibitor | | 31 | | | | | | | | |
| K93123848 | RAF265 | RAF inhibitor; VEGFR | ARMCX1 | armadillo repeat-containing | −0.20299 | 8.79E−05 | 0.005378 | −0.26747 | M | 0.101093 | M | 0.191571 |
| | | inhibitor | | x-linked protein 1 | | | | | | | | |
| K93123848 | RAF265 | RAF inhibitor; VEGFR | ATP8B2 | phospholipid-transporting | −0.18501 | 0.00036 | 0.013711 | −0.28343 | M | 0.101093 | M | 0.191571 |
| | | inhibitor | | atpase id | | | | | | | | |
| K93123848 | RAF265 | RAF inhibitor; VEGFR | CCDC136 | coiled-coil domain- | −0.18589 | 0.000337 | 0.013135 | −0.25096 | M | 0.101093 | M | 0.191571 |
| | | inhibitor | | containing protein 136 | | | | | | | | |
| K93123848 | RAF265 | RAF inhibitor; VEGFR | CHST10 | carbohydrate | −0.16637 | 0.00136 | 0.031538 | −0.30247 | M | 0.101093 | M | 0.191571 |
| | | inhibitor | | sulfotransferase 10 | | | | | | | | |
| K93123848 | RAF265 | RAF inhibitor; VEGFR | CNRIP1 | cb1 cannabinoid receptor- | −0.17278 | 0.000874 | 0.024004 | −0.25165 | M | 0.101093 | M | 0.191571 |
| | | inhibitor | | interacting protein 1 | | | | | | | | |
| K93123848 | RAF265 | RAF inhibitor; VEGFR | COL19A1 | collagen alpha-1 | −0.19598 | 0.000155 | 0.00787 | −0.25227 | M | 0.101093 | M | 0.191571 |
| | | inhibitor | | | | | | | | | | |
| K93123848 | RAF265 | RAF inhibitor; VEGFR | CYP2U1 | cytochrome p450 2u1 | −0.21352 | 3.63E−05 | 0.002948 | −0.29102 | M | 0.101093 | M | 0.191571 |
| | | inhibitor | | | | | | | | | | |
| K93123848 | RAF265 | RAF inhibitor; VEGFR | EPHA3 | ephrin type-a receptor 3 | −0.16609 | 0.001386 | 0.031919 | −0.28147 | M | 0.101093 | M | 0.191571 |
| | | inhibitor | | | | | | | | | | |
| K93123848 | RAF265 | RAF inhibitor; VEGFR | FAM180B | protein fam180b | −0.16123 | 0.001918 | 0.03878 | −0.24692 | M | 0.101093 | M | 0.191571 |
| | | inhibitor | | | | | | | | | | |
| K93123848 | RAF265 | RAF inhibitor; VEGFR | FGD1 | fyve; rhogef and ph domain- | −0.18426 | 0.000381 | 0.014197 | −0.25211 | M | 0.101093 | M | 0.191571 |
| | | inhibitor | | containing protein 1 | | | | | | | | |
| K93123848 | RAF265 | RAF inhibitor; VEGFR | GPR161 | g-protein coupled receptor | −0.1866 | 0.000319 | 0.012686 | −0.24182 | M | 0.101093 | M | 0.191571 |
| | | inhibitor | | 161 | | | | | | | | |
| K93123848 | RAF265 | RAF inhibitor; VEGFR | HMCN1 | hemicentin-1 | −0.18187 | 0.000454 | 0.015911 | −0.25492 | M | 0.101093 | M | 0.191571 |
| | | inhibitor | | | | | | | | | | |
| K93123848 | RAF265 | RAF inhibitor; VEGFR | IL16 | pro-interleukin-16 | −0.21421 | 3.42E−05 | 0.002828 | −0.28116 | M | 0.101093 | M | 0.191571 |
| | | inhibitor | | | | | | | | | | |
| K93123848 | RAF265 | RAF inhibitor; VEGFR | IRF4 | interferon regulatory factor | −0.15807 | 0.002357 | 0.043888 | −0.24649 | M | 0.101093 | M | 0.191571 |
| | | inhibitor | | 4 | | | | | | | | |
| K93123848 | RAF265 | RAF inhibitor; VEGFR | LIX1L | lix 1-like protein | −0.21023 | 4.81E−05 | 0.003575 | −0.2552 | M | 0.101093 | M | 0.191571 |
| | | inhibitor | | | | | | | | | | |
| K93123848 | RAF265 | RAF inhibitor; VEGFR | MCOLN2 | mucolipin-2 | −0.20922 | 5.24E−05 | 0.003792 | −0.27402 | M | 0.101093 | M | 0.191571 |
| | | inhibitor | | | | | | | | | | |
| K93123848 | RAF265 | RAF inhibitor; VEGFR | NRROS | transforming growth factor | −0.21782 | 2.5E−05 | 0.002273 | −0.27097 | M | 0.101093 | M | 0.191571 |
| | | inhibitor | | beta activator lrrc33 | | | | | | | | |
| K93123848 | RAF265 | RAF inhibitor; VEGFR | PRKD1 | | −0.20553 | 7.13E−05 | 0.004684 | −0.29262 | M | 0.101093 | M | 0.191571 |
| | | inhibitor | | | | | | | | | | |
| K93123848 | RAF265 | RAF inhibitor; VEGFR | PYGO1 | py gopus homolog 1 | −0.20407 | 8.05E−05 | 0.005071 | −0.27335 | M | 0.101093 | M | 0.191571 |
| | | inhibitor | | | | | | | | | | |
| K93123848 | RAF265 | RAF inhibitor; VEGFR | RHOJ | rho-related gtp-binding | −0.21861 | 2.33E−05 | 0.002164 | −0.2411 | M | 0.101093 | M | 0.191571 |
| | | inhibitor | | protein rhoj | | | | | | | | |
| K93123848 | RAF265 | RAF inhibitor; VEGFR | RUNX3 | runt-related transcription | −0.16417 | 0.001577 | 0.034497 | −0.24933 | M | 0.101093 | M | 0.191571 |
| | | inhibitor | | factor 3 | | | | | | | | |
| K93123848 | RAF265 | RAF inhibitor; VEGFR | SGCD | delta-sarcoglycan | −0.17142 | 0.000961 | 0.025493 | −0.24557 | M | 0.101093 | M | 0.191571 |
| | | inhibitor | | | | | | | | | | |
| K93123848 | RAF265 | RAF inhibitor; VEGFR | SMO | smoothened homolog | −0.19623 | 0.000152 | 0.007777 | −0.307 | M | 0.101093 | M | 0.191571 |
| | | inhibitor | | | | | | | | | | |
| K93123848 | RAF265 | RAF inhibitor; VEGFR | SPART | spartin | −0.1863 | 0.000327 | 0.012888 | −0.25979 | M | 0.101093 | M | 0.191571 |
| | | inhibitor | | | | | | | | | | |
| K93123848 | RAF265 | RAF inhibitor; VEGFR | WIPF1 | was/wasl-interacting protein | −0.19352 | 0.000188 | 0.008961 | −0.24425 | M | 0.101093 | M | 0.191571 |
| | | inhibitor | | family member 1 | | | | | | | | |
| K94455792 | ICG-001 | beta-catenin inhibitor | CSPG4 | chondroitin sulfate | −0.19632 | 0.000157 | 0.007939 | −0.23968 | M | 0.178329 | M | 0.163484 |
| | | | | proteoglycan 4 | | | | | | | | |
| K94455792 | ICG-001 | beta-catenin inhibitor | MYH10 | myosin-10 | −0.1641 | 0.001632 | 0.035219 | −0.27195 | M | 0.178329 | M | 0.163484 |
| K94455792 | ICG-001 | beta-catenin inhibitor | RCN2 | reticulocalbin-2 | −0.18914 | 0.000274 | 0.011481 | −0.2527 | M | 0.178329 | M | 0.163484 |
| K94455792 | ICG-001 | beta-catenin inhibitor | TRPV2 | transient receptor potential | −0.17678 | 0.000681 | 0.020514 | −0.26506 | M | 0.178329 | M | 0.163484 |
| | | | | cation channel subfamily v | | | | | | | | |
| | | | | member 2 | | | | | | | | |
| K08542803 | gambogic-acid | caspase activator | FRMD6 | ferm domain-containing | −0.19335 | 0.00021 | 0.030901 | −0.31589 | M | −0.03027 | M | −0.00196 |
| | | | | protein 6 | | | | | | | | |
| K09951645 | dabrafenib | RAF inhibitor | ASB2 | ankyrin repeat and socs box | −0.25863 | 8.69E−07 | 0.001428 | −0.39736 | M | 0.043133 | M | 0.14386 |
| | | | | protein 2 | | | | | | | | |
| K09951645 | dabrafenib | RAF inhibitor | BCL2A1 | bcl-2-related protein a1 | −0.29464 | 1.76E−08 | 0.000133 | −0.41865 | M | 0.043133 | M | 0.14386 |
| K09951645 | dabrafenib | RAF inhibitor | CDH19 | cadherin-19 | −0.21686 | 4.08E−05 | 0.012828 | −0.31348 | M | 0.043133 | M | 0.14386 |
| K09951645 | dabrafenib | RAF inhibitor | CHST11 | carbohydrate | −0.19116 | 0.00031 | 0.03747 | −0.30972 | M | 0.043133 | M | 0.14386 |
| | | | | sulfotransferase 11 | | | | | | | | |
| K09951645 | dabrafenib | RAF inhibitor | COL19A1 | collagen alpha-1 | −0.29259 | 2.23E−08 | 0.000153 | −0.40195 | M | 0.043133 | M | 0.14386 |
| K09951645 | dabrafenib | RAF inhibitor | DAAM2 | disheveled-associated | −0.2037 | 0.000119 | 0.023095 | −0.30795 | M | 0.043133 | M | 0.14386 |
| | | | | activator of morphogenesis 2 | | | | | | | | |
| K09951645 | dabrafenib | RAF inhibitor | EDNRB | endothelin receptor type b | −0.2923 | 2.31E−08 | 0.000154 | −0.36828 | M | 0.043133 | M | 0.14386 |
| K09951645 | dabrafenib | RAF inhibitor | FCGR2A | low affinity | −0.2823 | 7.14E−08 | 0.000321 | −0.30776 | M | 0.043133 | M | 0.14386 |
| | | | | immunoglobulin gamma fc | | | | | | | | |
| | | | | region receptor ii-a-related | | | | | | | | |
| K09951645 | dabrafenib | RAF inhibitor | FCRLA | fc receptor-like a | −0.32782 | 2.91E−10 | 1E−05 | −0.39273 | M | 0.043133 | M | 0.14386 |
| K09951645 | dabrafenib | RAF inhibitor | GAS7 | growth arrest-specific | −0.22244 | 2.54E−05 | 0.009936 | −0.37622 | M | 0.043133 | M | 0.14386 |
| | | | | protein 7 | | | | | | | | |
| K09951645 | dabrafenib | RAF inhibitor | GYPC | glycophorin-c | −0.30953 | 2.98E−09 | 4.47E−05 | −0.30487 | M | 0.043133 | M | 0.14386 |
| K09951645 | dabrafenib | RAF inhibitor | HPS5 | hermansky-pudlak | −0.21148 | 6.36E−05 | 0.016397 | −0.30601 | M | 0.043133 | M | 0.14386 |
| | | | | syndrome 5 protein | | | | | | | | |
| K09951645 | dabrafenib | RAF inhibitor | IL16 | pro-interleukin-16 | −0.26016 | 7.46E−07 | 0.001299 | −0.39859 | M | 0.043133 | M | 0.14386 |
| K09951645 | dabrafenib | RAF inhibitor | IRF4 | interferon regulatory factor 4 | −0.27643 | 1.36E−07 | 0.000479 | −0.33551 | M | 0.043133 | M | 0.14386 |
| K09951645 | dabrafenib | RAF inhibitor | ITGB3 | integrin beta-3 | −0.22968 | 1.35E−05 | 0.006964 | −0.35606 | M | 0.043133 | M | 0.14386 |
| K09951645 | dabrafenib | RAF inhibitor | LPXN | leupaxin | −0.2402 | 5.18E−06 | 0.004057 | −0.31391 | M | 0.043133 | M | 0.14386 |
| K09951645 | dabrafenib | RAF inhibitor | MIA | melanoma-derived growth | −0.26532 | 4.4E−07 | 0.000942 | −0.37161 | M | 0.043133 | M | 0.14386 |
| | | | | regulatory protein | | | | | | | | |
| K09951645 | dabrafenib | RAF inhibitor | NFATC2 | nuclear factor of activated t- | −0.26331 | 5.41E−07 | 0.001055 | −0.32185 | M | 0.043133 | M | 0.14386 |
| | | | | cells; cytoplasmic 2 | | | | | | | | |
| K09951645 | dabrafenib | RAF inhibitor | PLP1 | myelin proteolipid protein | −0.30462 | 5.41E−09 | 6.64E−05 | −0.35707 | M | 0.043133 | M | 0.14386 |
| K09951645 | dabrafenib | RAF inhibitor | PTCRA | pre t-cell antigen receptor | −0.30249 | 6.99E−09 | 7.87E−05 | −0.41346 | M | 0.043133 | M | 0.14386 |
| | | | | alpha | | | | | | | | |
| K09951645 | dabrafenib | RAF inhibitor | SHROOM4 | protein shroom4 | −0.34578 | 2.54E−11 | 2.08E−06 | −0.34358 | M | 0.043133 | M | 0.14386 |
| K09951645 | dabrafenib | RAF inhibitor | SRPX | sushi repeat-containing | −0.23172 | 1.12E−05 | 0.006318 | −0.3477 | M | 0.043133 | M | 0.14386 |
| | | | | protein srpx | | | | | | | | |
| K09951645 | dabrafenib | RAF inhibitor | STK10 | serine/threonine-protein | −0.20043 | 0.000153 | 0.02638 | −0.33469 | M | 0.043133 | M | 0.14386 |
| | | | | kinase 10 | | | | | | | | |
| K09951645 | dabrafenib | RAF inhibitor | TRPV2 | transient receptor potential | −0.30149 | 7.87E−09 | 8.51E−05 | −0.35302 | M | 0.043133 | M | 0.14386 |
| | | | | cation channel subfamily v | | | | | | | | |
| | | | | member 2 | | | | | | | | |
| K09951645 | dabrafenib | RAF inhibitor | WWTR1 | ww domain-containing | −0.24661 | 2.83E−06 | 0.002923 | −0.34605 | M | 0.043133 | M | 0.14386 |
| | | | | transcription regulator | | | | | | | | |
| | | | | protein 1 | | | | | | | | |
| K13169950 | NSC-3852 | HDAC inhibitor | MPP2 | forkhead box protein m1 | −0.19379 | 0.000212 | 0.031005 | −0.29806 | M | 0.093556 | M | −0.00012 |
| K25970317 | resibufogenin | Na/K-ATPase inhibitor | B4GALNT1 | beta-1; 4 n- | −0.25891 | 9.09E−07 | 0.001476 | −0.31383 | MSL | −0.05263 | MSL | −0.09203 |
| | | | | acetylgalactosaminyl- | | | | | | | | |
| | | | | transferase 1 | | | | | | | | |
| K25970317 | resibufogenin | Na/K-ATPase inhibitor | BEND6 | ben domain-containing | −0.20376 | 0.000124 | 0.023598 | −0.33503 | MSL | −0.05263 | MSL | −0.09203 |
| | | | | protein 6 | | | | | | | | |
| K25970317 | resibufogenin | Na/K-ATPase inhibitor | DCLK2 | serine/threonine-protein | −0.25035 | 2.11E−06 | 0.002505 | −0.29911 | MSL | −0.05263 | MSL | −0.09203 |
| | | | | kinase dclk2 | | | | | | | | |
| K25970317 | resibufogenin | Na/K-ATPase inhibitor | FERMT2 | fermitin family homolog 2 | −0.27164 | 2.46E−07 | 0.000679 | −0.34523 | MSL | −0.05263 | MSL | −0.09203 |
| K25970317 | resibufogenin | Na/K-ATPase inhibitor | FHL1 | four and a half lim domains | −0.22738 | 1.75E−05 | 0.00807 | −0.39527 | MSL | −0.05263 | MSL | −0.09203 |
| | | | | protein 1 | | | | | | | | |
| K25970317 | resibufogenin | Na/K-ATPase inhibitor | LRRC8C | volume-regulated anion | −0.21149 | 6.67E−05 | 0.016817 | −0.29933 | MSL | −0.05263 | MSL | −0.09203 |
| | | | | channel subunit lrrc8c | | | | | | | | |
| K25970317 | resibufogenin | Na/K-ATPase inhibitor | MRC2 | c-type mannose receptor 2 | −0.25267 | 1.68E−06 | 0.002166 | −0.33962 | MSL | −0.05263 | MSL | −0.09203 |
| K25970317 | resibufogenin | Na/K-ATPase inhibitor | MSRB3 | methionine-r-sulfoxide | −0.26092 | 7.42E−07 | 0.001299 | −0.38269 | MSL | −0.05263 | MSL | −0.09203 |
| | | | | reductase b3 | | | | | | | | |
| K25970317 | resibufogenin | Na/K-ATPase inhibitor | SYDE1 | rho gtpase-activating protein | −0.29869 | 1.21E−08 | 0.000107 | −0.30644 | MSL | −0.05263 | MSL | −0.09203 |
| | | | | syde1 | | | | | | | | |
| K25970317 | resibufogenin | Na/K-ATPase inhibitor | TLCD5 | tlc domain-containing | −0.23238 | 1.12E−05 | 0.006318 | −0.37712 | MSL | −0.05263 | MSL | −0.09203 |
| | | | | protein 5 | | | | | | | | |
| K25970317 | resibufogenin | Na/K-ATPase inhibitor | TRPC1 | short transient receptor | −0.27139 | 2.52E−07 | 0.000695 | −0.33298 | MSL | −0.05263 | MSL | −0.09203 |
| | | | | potential channel 1 | | | | | | | | |
| K25970317 | resibufogenin | Na/K-ATPase inhibitor | ZEB1 | zinc finger e-box-binding | −0.26988 | 2.95E−07 | 0.000761 | −0.31193 | MSL | −0.05263 | MSL | −0.09203 |
| | | | | homeobox 1 | | | | | | | | |
| K61443650 | thiomersal | other antibiotic | FMNL3 | formin-like protein 3 | −0.23623 | 4.62E−06 | 0.003823 | −0.3199 | M | −0.05708 | M | 0.028697 |
| K61443650 | thiomersal | other antibiotic | PIK3CD | phosphatidylinositol 4; 5- | −0.23165 | 7.12E−06 | 0.004948 | −0.31557 | M | −0.05708 | M | 0.028697 |
| | | | | bisphosphate 3-kinase | | | | | | | | |
| | | | | catalytic subunit delta | | | | | | | | |
| | | | | isoform | | | | | | | | |
| K61443650 | thiomersal | other antibiotic | PRTG | protogenin | −0.22174 | 1.76E−05 | 0.008119 | −0.30441 | M | −0.05708 | M | 0.028697 |
| K84868168 | erdafitinib | FGFR inhibitor | ATP8B2 | phospholipid-transporting | −0.19857 | 0.000185 | 0.029047 | −0.30762 | MSL | −0.0744 | MSL/M | 0.006092 |
| | | | | atpase id | | | | | | | | |
| K84868168 | erdafitinib | FGFR inhibitor | CNTNAP1 | contactin-associated protein 1 | −0.25595 | 1.22E−06 | 0.00177 | −0.3174 | MSL | −0.0744 | MSL/M | 0.006092 |
| K84868168 | erdafitinib | FGFR inhibitor | DCLK2 | serine/threonine-protein | −0.23933 | 5.97E−06 | 0.004446 | −0.31828 | MSL | −0.0744 | MSL/M | 0.006092 |
| | | | | kinase dclk2 | | | | | | | | |
| K84868168 | erdafitinib | FGFR inhibitor | FBN1 | fibrillin-1 | −0.2825 | 7.62E−08 | 0.000328 | −0.30056 | MSL | −0.0744 | MSL/M | 0.006092 |
| K84868168 | erdafitinib | FGFR inhibitor | FGFR1 | fibroblast growth factor | −0.31842 | 1.09E−09 | 2.55E−05 | −0.35083 | MSL | −0.0744 | MSL/M | 0.006092 |
| | | | | receptor 1 | | | | | | | | |
| K84868168 | erdafitinib | FGFR inhibitor | FHL1 | four and a half lim domains | −0.19794 | 0.000194 | 0.029783 | −0.30652 | MSL | −0.0744 | MSL/M | 0.006092 |
| | | | | protein 1 | | | | | | | | |
| K84868168 | erdafitinib | FGFR inhibitor | GLI1 | zinc finger protein gli1 | −0.20185 | 0.000144 | 0.025558 | −0.33185 | MSL | −0.0744 | MSL/M | 0.006092 |
| K84868168 | erdafitinib | FGFR inhibitor | GLIPR2 | golgi-associated plant | −0.31212 | 2.4E−09 | 4.11E−05 | −0.31536 | MSL | −0.0744 | MSL/M | 0.006092 |
| | | | | pathogenesis-related protein 1 | | | | | | | | |
| K84868168 | erdafitinib | FGFR inhibitor | MAP1B | microtubule-associated | −0.24434 | 3.74E−06 | 0.003398 | −0.32118 | MSL | −0.0744 | MSL/M | 0.006092 |
| | | | | protein 1b | | | | | | | | |
| K84868168 | erdafitinib | FGFR inhibitor | MEX3B | rna-binding protein mex3b | −0.19734 | 0.000203 | 0.030421 | −0.31704 | MSL | −0.0744 | MSL/M | 0.006092 |
| K84868168 | erdafitinib | FGFR inhibitor | MRAS | ras-related protein m-ras | −0.26462 | 5.1E−07 | 0.001031 | −0.30048 | MSL | −0.0744 | MSL/M | 0.006092 |
| K84868168 | erdafitinib | FGFR inhibitor | MRC2 | c-type mannose receptor 2 | −0.20138 | 0.000149 | 0.025983 | −0.36328 | MSL | −0.0744 | MSL/M | 0.006092 |
| K84868168 | erdafitinib | FGFR inhibitor | MSRB3 | methionine-r-sulfoxide | −0.29951 | 1.1E−08 | 0.000104 | −0.31656 | MSL | −0.0744 | MSL/M | 0.006092 |
| | | | | reductase b3 | | | | | | | | |
| K84868168 | erdafitinib | FGFR inhibitor | NLGN2 | neuroligin-2 | −0.2421 | 4.62E−06 | 0.003823 | −0.3305 | MSL | −0.0744 | MSL/M | 0.006092 |
| K84868168 | erdafitinib | FGFR inhibitor | NREP | neuronal regeneration- | −0.19768 | 0.000198 | 0.030069 | −0.32394 | MSL | −0.0744 | MSL/M | 0.006092 |
| | | | | related protein | | | | | | | | |
| K84868168 | erdafitinib | FGFR inhibitor | PDGFRB | platelet-derived growth | −0.33939 | 6.98E−11 | 3.82E−06 | −0.3048 | MSL | −0.0744 | MSL/M | 0.006092 |
| | | | | factor receptor beta | | | | | | | | |
| K84868168 | erdafitinib | FGFR inhibitor | RBPMS2 | rna-binding protein with | −0.19068 | 0.000334 | 0.038903 | −0.3019 | MSL | −0.0744 | MSL/M | 0.006092 |
| | | | | multiple splicing 2 | | | | | | | | |
| K84868168 | erdafitinib | FGFR inhibitor | SYDE1 | rho gtpase-activating protein | −0.27347 | 2.02E−07 | 0.000615 | −0.29962 | MSL | −0.0744 | MSL/M | 0.006092 |
| | | | | syde1 | | | | | | | | |
| K84868168 | erdafitinib | FGFR inhibitor | TTC28 | tetratricopeptide repeat | −0.2646 | 5.11E−07 | 0.001031 | −0.31995 | MSL | −0.0744 | MSL/M | 0.006092 |
| | | | | protein 28 | | | | | | | | |
| K84868168 | erdafitinib | FGFR inhibitor | TUBA1A | tubulin alpha-1a chain | −0.22155 | 2.89E−05 | 0.010639 | −0.3389 | MSL | −0.0744 | MSL/M | 0.006092 |
| K84868168 | erdafitinib | FGFR inhibitor | VASH1 | tubulinyl-tyr | −0.19412 | 0.000259 | 0.034218 | −0.33053 | MSL | −0.0744 | MSL/M | 0.006092 |
| | | | | carboxypeptidase 1 | | | | | | | | |
| K84868168 | erdafitinib | FGFR inhibitor | VIM | vimentin | −0.26067 | 7.61E−07 | 0.001311 | −0.32641 | MSL | −0.0744 | MSL/M | 0.006092 |
| K84868168 | erdafitinib | FGFR inhibitor | ZEB1 | zinc finger e-box-binding | −0.27392 | 1.93E−07 | 0.000598 | −0.31151 | MSL | −0.0744 | MSL/M | 0.006092 |
| | | | | homeobox 1 | | | | | | | | |
| K94441233 | mevastatin | HMGCR inhibitor | CAVIN1 | caveolae-associated protein 1 | −0.27085 | 3.38E−07 | 0.000819 | −0.33251 | MSL | −0.00119 | MSL | −0.09323 |
| K94441233 | mevastatin | HMGCR inhibitor | CCDC80 | coiled-coil domain- | −0.21025 | 8.52E−05 | 0.01925 | −0.31342 | MSL | −0.00119 | MSL | −0.09323 |
| | | | | containing protein 80 | | | | | | | | |
| K94441233 | mevastatin | HMGCR inhibitor | COL13A1 | collagen alpha-1 | −0.20335 | 0.000146 | 0.02572 | −0.34946 | MSL | −0.00119 | MSL | −0.09323 |
| K94441233 | mevastatin | HMGCR inhibitor | EVC | ellis-van creveld syndrome | −0.30251 | 1.03E−08 | 0.000104 | −0.3209 | MSL | −0.00119 | MSL | −0.09323 |
| | | | | protein | | | | | | | | |
| K94441233 | mevastatin | HMGCR inhibitor | HEG1 | protein heg homolog 1 | −0.2727 | 2.79E−07 | 0.000738 | −0.36346 | MSL | −0.00119 | MSL | −0.09323 |
| K94441233 | mevastatin | HMGCR inhibitor | LAYN | layilin | −0.23458 | 1.1E−05 | 0.006258 | −0.31407 | MSL | −0.00119 | MSL | −0.09323 |
| K94441233 | mevastatin | HMGCR inhibitor | MAP3K3 | mitogen-activated protein | −0.20686 | 0.000111 | 0.022463 | −0.30851 | MSL | −0.00119 | MSL | −0.09323 |
| | | | | kinase kinase kinase 3 | | | | | | | | |
| K94441233 | mevastatin | HMGCR inhibitor | MCAM | cell surface glycoprotein | −0.22153 | 3.39E−05 | 0.01158 | −0.31283 | MSL | −0.00119 | MSL | −0.09323 |
| | | | | muc18 | | | | | | | | |
| K94441233 | mevastatin | HMGCR inhibitor | PIK3CD | phosphatidylinositol 4; 5- | −0.21451 | 6.05E−05 | 0.016012 | −0.30633 | MSL | −0.00119 | MSL | −0.09323 |
| | | | | bisphosphate 3-kinase | | | | | | | | |
| | | | | catalytic subunit delta | | | | | | | | |
| | | | | isoform | | | | | | | | |

TABLE 24B
Mechanism of action and associated subtype for selected
compounds with significant subtype association.

| BROAD | | | | Associated | compound | compound |
|---|---|---|---|---|---|---|
| compound | Compound | | Associated | Gene | gene train | gene train |
| ID | Name | MoA | Gene | Family | corr | pvalue |
| K09951645 | dabrafenib | RAF inhibitor | SH2D1B | sh2 domain- | −0.11236 | 0.035098 |
| | | | | containing | | |
| | | | | protein 1b | | |
| K09951645 | dabrafenib | RAF inhibitor | EVC2 | limbin | −0.12568 | 0.018328 |
| K20285085 | R406 | SYK inhibitor | CRMP1 | dihydro- | −0.1488 | 0.005151 |
| | | | | pyrimidinase- | | |
| | | | | related | | |
| | | | | protein 1 | | |
| K72327355 | baicalein | lipoxygenase | PRICKLE2 | prickle-like | −0.15967 | 0.002312 |
| | | inhibitor | | protein 2 | | |
| K39974922 | lenvatinib | FGFR inhibitor; | DCLK2 | serine/ | −0.20098 | 0.000132 |
| | | KIT inhibitor; | | threonine- | | |
| | | PDGFR tyrosine | | protein | | |
| | | kinase receptor | | kinase | | |
| | | inhibitor; | | dclk2 | | |
| | | VEGFR inhibitor | | | | |
| K84868168 | erdafitinib | FGFR inhibitor | EBF1 | transcription | −0.10611 | 0.047295 |
| | | | | factor coel | | |
| A00842753 | oleuropein | estrogen | FBN1 | fibrillin-1 | −0.11021 | 0.039319 |
| | | receptor | | | | |
| | | agonist | | | | |
| K20285085 | R406 | SYK inhibitor | FHL1 | four and a | −0.25729 | 9.95E−07 |
| | | | | half lim | | |
| | | | | domains | | |
| | | | | protein 1 | | |
| K39974922 | lenvatinib | FGFR inhibitor; | NFASC | neurofascin | −0.15569 | 0.003184 |
| | | KIT inhibitor; | | | | |
| | | PDGFR tyrosine | | | | |
| | | kinase receptor | | | | |
| | | inhibitor, | | | | |
| | | VEGFR inhibitor | | | | |
| K94441233 | mevastatin | HMGCR inhibitor | ABL1 | tyrosine- | −0.12545 | 0.01994 |
| | | | | protein | | |
| | | | | kinase abl1 | | |
| K75958547 | pitavastatin | HMGCR inhibitor | IL4I1 | 1-amino-acid | −0.10707 | 0.040092 |
| | | | | oxidase | | |
| K17203476 | LY2874455 | FGFR antagonist | CAVIN1 | caveolae- | −0.19016 | 0.000244 |
| | | | | associated | | |
| | | | | protein 1 | | |
| K09951645 | dabrafenib | RAF inhibitor | LCP2 | lymphocyte | −0.15397 | 0.003783 |
| | | | | cytosolic | | |
| | | | | protein 2 | | |
| K75958547 | pitavastatin | HMGCR inhibitor | LTBP2 | | −0.16591 | 0.001403 |
| K08542803 | gambogic- | caspase | MICB | mhc class i | −0.16383 | 0.001738 |
| | acid | activator | | polypeptide- | | |
| | | | | related | | |
| | | | | sequence | | |
| | | | | a-related | | |
| K02113016 | olaparib | PARP inhibitor | POLR2A | dna-directed rna | −0.22693 | 1.3E−05 |
| | | | | polymerase ii | | |
| | | | | subunit rpb1 | | |
| K13044802 | ciclopirox | membrane | POLR2A | dna-directed rna | −0.12618 | 0.015437 |
| | | integrity | | polymerase ii | | |
| | | inhibitor | | subunit rpb1 | | |
| K13169950 | NSC-3852 | HDAC inhibitor | POLR2A | dna-directed rna | −0.12525 | 0.017274 |
| | | | | polymerase ii | | |
| | | | | subunit rpb1 | | |
| K54955827 | niraparib | PARP inhibitor | POLR2A | dna-directed rna | −0.12615 | 0.017891 |
| | | | | polymerase ii | | |
| | | | | subunit rpb1 | | |
| K75958547 | pitavastatin | HMGCR inhibitor | PTPN14 | tyrosine-protein | −0.22104 | 1.88E−05 |
| | | | | phosphatase non- | | |
| | | | | receptor type 14 | | |
| K03289018 | CCT137690 | Aurora kinase | PTPRG | receptor-type | −0.16073 | 0.00201 |
| | | inhibitor | | tyrosine-protein | | |
| | | | | phosphatase | | |
| | | | | gamma | | |
| K54955827 | niraparib | PARP inhibitor | FAT4 | protocadherin | −0.11104 | 0.037319 |
| | | | | fat 4 | | |
| K61688984 | RGFP966 | HDAC inhibitor | LIMD2 | | −0.17338 | 0.000866 |
| K75958547 | pitavastatin | HMGCR inhibitor | RASSF4 | ras and rab | −0.13101 | 0.011889 |
| | | | | interactor 2 | | |
| K84868168 | erdafitinib | FGFR inhibitor | CACNA1G | voltage-dependent | −0.11953 | 0.025334 |
| | | | | t-type calcium | | |
| | | | | channel subunit | | |
| | | | | alpha-1g | | |
| K13169950 | NSC-3852 | HDAC inhibitor | ADAMTSL1 | | −0.15882 | 0.002476 |
| K84868168 | erdafitinib | FGFR inhibitor | NREP | neuronal | −0.19768 | 0.000198 |
| | | | | regeneration- | | |
| | | | | related protein | | |
| A19736161 | ondansetron | serotonin | EPSTI1 | epithelial- | −0.14488 | 0.005359 |
| | | receptor | | stromal | | |
| | | antagonist | | interaction | | |
| | | | | protein 1 | | |
| K79821389 | rubitecan | topoisomerase | EBI3 | interleukin-27 | −0.11499 | 0.031497 |
| | | inhibitor | | subunit beta | | |
| K67578145 | GDC-0879 | RAF inhibitor | TNFSF13B | tumor necrosis | −0.11464 | 0.03354 |
| | | | | factor ligand | | |
| | | | | superfamily | | |
| | | | | member 13b | | |
| K19333160 | RKI-1447 | rho associated | FBXO17 | f-box only | −0.18772 | 0.000305 |
| | | kinase | | protein 17 | | |
| | | inhibitor | | | | |
| K67578145 | GDC-0879 | RAF inhibitor | SH2D1B | sh2 domain- | −0.13502 | 0.012187 |
| | | | | containing | | |
| | | | | protein 1b | | |
| K94455792 | ICG-001 | beta-catenin | EDNRB | endothelin | −0.18843 | 0.000289 |
| | | inhibitor | | receptor type b | | |
| K19687926 | lapatinib | EGFR inhibitor | EPHB3 | ephrin type-b | −0.14608 | 0.005046 |
| | | | | receptor 3 | | |
| K42805893 | osimertinib | EGFR inhibitor | EPHB3 | ephrin type-b | −0.14965 | 0.004011 |
| | | | | receptor 3 | | |
| K46386702 | ARRY-334543 | EGFR inhibitor | EPHB3 | ephrin type-b | −0.14245 | 0.006482 |
| | | | | receptor 3 | | |
| K05804044 | AZ-628 | RAF inhibitor | FLT1 | vascular | −0.30989 | 1.53E−09 |
| | | | | endothelial | | |
| | | | | growth factor | | |
| | | | | receptor 1 | | |
| K13394247 | radafaxine | dopamine | SYNM | asparagine--trna | −0.11637 | 0.029509 |
| | | norepinephrine | | ligase; | | |
| | | reuptake | | mitochondrial- | | |
| | | inhibitor | | related | | |
| K12040459 | AT7867 | AKT inhibitor | RCOR2 | rest | −0.16454 | 0.001633 |
| | | | | corepressor 2 | | |
| A12230535 | nutlin-3 | MDM inhibitor | HSPG2 | basement | −0.12664 | 0.017451 |
| | | | | membrane- | | |
| | | | | specific heparan | | |
| | | | | sulfate | | |
| | | | | proteoglycan | | |
| | | | | core protein | | |
| K81332461 | maxacalcitol | vitamin D | ITGB4 | integrin beta-4 | −0.13978 | 0.008639 |
| | | receptor | | | | |
| | | agonist | | | | |
| K31866293 | TAK-632 | RAF inhibitor | NRROS | transforming | −0.24743 | 1.65E−06 |
| | | | | growth factor | | |
| | | | | beta activator | | |
| | | | | lrrc33 | | |
| K56981171 | brigatinib | ALK tyrosine | KRT17 | keratin; type i | −0.26518 | 4.81E−07 |
| | | kinase receptor | | cytoskeletal 17 | | |
| | | inhibitor; | | | | |
| | | EGFR inhibitor | | | | |
| A56085258 | LGX818 | RAF inhibitor | LCP2 | lymphocyte | −0.19511 | 0.000166 |
| | | | | cytosolic | | |
| | | | | protein 2 | | |
| K26818574 | BIX-01294 | histone lysine | NGFR | tumor necrosis | −0.15854 | 0.002351 |
| | | methyl- | | factor receptor | | |
| | | transferase | | superfamily | | |
| | | inhibitor | | member 16 | | |
| K12040459 | AT7867 | AKT inhibitor | CLEC1A | c-type lectin | −0.13105 | 0.012334 |
| | | | | domain family 1 | | |
| | | | | member a | | |
| A56085258 | LGX818 | RAF inhibitor | PLXNB3 | plexin-b3 | −0.18322 | 0.000411 |
| K05804044 | AZ-628 | RAF inhibitor | PLXNB3 | plexin-b3 | −0.22015 | 2.26E−05 |
| K31866293 | TAK-632 | RAF inhibitor | PLXNB3 | plexin-b3 | −0.11197 | 0.032232 |
| K13183738 | pentamidine | anti- | POLR2A | dna-directed rna | −0.15586 | 0.002867 |
| | | pneumocystis | | polymerase ii | | |
| | | agent | | subunit rpb1 | | |
| K29905972 | axitinib | PDGFR tyrosine | POLR2A | dna-directed rna | −0.13705 | 0.00913 |
| | | kinase receptor | | polymerase ii | | |
| | | inhibitor; VEGFR | | subunit rpb1 | | |
| | | inhibitor | | | | |
| K47150025 | KI-8751 | KIT inhibitor; | POLR2A | dna-directed rna | −0.11823 | 0.023693 |
| | | PDGFR tyrosine | | polymerase ii | | |
| | | kinase receptor | | subunit rpb1 | | |
| | | inhibitor; | | | | |
| | | VEGFR inhibitor | | | | |
| K77791657 | 3- | histone lysine | PSMA3 | proteasome | −0.18919 | 0.000267 |
| | deazaneplanocin- | methyl- | | subunit alpha | | |
| | A | transferase | | type-3 | | |
| | | inhibitor | | | | |
| K16180792 | bis(maltolato) | tyrosine | PTPN14 | tyrosine-protein | −0.10809 | 0.042695 |
| | oxovanadium | phosphatase | | phosphatase non- | | |
| | (IV) | inhibitor | | receptor type 14 | | |
| K31866293 | TAK-632 | RAF inhibitor | BCL2A1 | bcl-2-related | −0.23617 | 4.93E−06 |
| | | | | protein a1 | | |
| K19687926 | lapatinib | EGFR inhibitor | TMPRSS3 | transmembrane | −0.12707 | 0.014857 |
| | | | | protease | | |
| | | | | serine 4 | | |
| K37561857 | zardaverine | phosphodi- | TACC1 | transforming | −0.13693 | 0.009586 |
| | | esterase | | acidic coiled- | | |
| | | inhibitor | | coil-containing | | |
| | | | | protein 1 | | |
| K93123848 | RAF265 | RAF inhibitor; | CNTNAP1 | contactin- | −0.19589 | 0.000156 |
| | | VEGFR inhibitor | | associated | | |
| | | | | protein 1 | | |
| A75975749 | bafetinib | Bcr-Abl kinase | LPXN | leupaxin | −0.2009 | 0.000155 |
| | | inhibitor; LYN | | | | |
| | | tyrosine kinase | | | | |
| | | inhibitor | | | | |
| K94455792 | ICG-001 | beta-catenin | SH3PXD2A | sh3 and px | −0.13274 | 0.011019 |
| | | inhibitor | | domain- | | |
| | | | | containing | | |
| | | | | protein 2a | | |

| | | compound | compound | compound | | | | |
|---|---|---|---|---|---|---|---|---|
| BROAD | compound | gene dep | gene dep | gene dep | TNB | DTIO | TNB | DTIO |
| compound | gene test | train | train | test | CType | Corr | CType | Corr |
| ID | corr | corr | pvalue | corr | train | train | test | test |
| K09951645 | −0.25099 | −0.20891 | 0.000699 | −0.24599 | M | 0.043133 | M | 0.14386 |
| K09951645 | −0.15596 | −0.14925 | 0.040389 | −0.24637 | M | 0.043133 | M | 0.14386 |
| K20285085 | −0.15157 | −0.15198 | 0.036823 | −0.26917 | MSL | −0.1076 | MSL | −0.04153 |
| K72327355 | −0.22131 | −0.20953 | 0.021081 | −0.23851 | IM | −0.14417 | IM | −0.04244 |
| K39974922 | −0.26734 | −0.13985 | 0.022271 | −0.17941 | MSL | −0.03256 | M | 0.002695 |
| K84868168 | −0.1734 | −0.13163 | 0.03252 | −0.1897 | MSL | −0.0744 | M | 0.006092 |
| A00842753 | −0.19084 | −0.16182 | 0.024937 | −0.19376 | MSL | −0.00213 | MSL | 0.021715 |
| K20285085 | −0.19191 | −0.21511 | 0.019323 | −0.26019 | MSL | −0.1076 | MSL | −0.04153 |
| K39974922 | −0.16642 | −0.13017 | 0.044397 | −0.18236 | MSL | −0.03256 | M | 0.002695 |
| K94441233 | −0.14942 | −0.14081 | 0.023967 | −0.2168 | MSL | −0.00119 | MSL | −0.09323 |
| K75958547 | −0.25368 | −0.22129 | 0.001778 | −0.21053 | MSL | −0.04476 | M | −0.0837 |
| K17203476 | −0.20469 | −0.18906 | 0.007798 | −0.24234 | IM | −0.14489 | MSL | −0.0838 |
| K09951645 | −0.17003 | −0.25728 | 2.77E−05 | −0.30414 | M | 0.043133 | M | 0.14386 |
| K75958547 | −0.25061 | −0.1581 | 0.026493 | −0.22687 | MSL | −0.04476 | M | −0.0837 |
| K08542803 | −0.205 | −0.12811 | 0.035033 | −0.17798 | M | −0.03027 | M | −0.00196 |
| K02113016 | −0.2403 | −0.12235 | 0.044173 | −0.21218 | M | 0.026605 | M | 0.064041 |
| K13044802 | −0.17936 | −0.14154 | 0.018859 | −0.26998 | IM | −0.15742 | IM | −0.06749 |
| K13169950 | −0.20879 | −0.12204 | 0.045123 | −0.24502 | M | 0.093556 | M | −0.00012 |
| K54955827 | −0.2096 | −0.13507 | 0.027622 | −0.21753 | M | 0.035768 | M | −0.03477 |
| K75958547 | −0.28287 | −0.12737 | 0.034757 | −0.16688 | MSL | −0.04476 | M | −0.0837 |
| K03289018 | −0.19761 | −0.1761 | 0.003449 | −0.16896 | M | −0.02552 | M | −0.13958 |
| K54955827 | −0.16653 | −0.23812 | 0.000827 | −0.19313 | M | 0.035768 | M | −0.03477 |
| K61688984 | −0.21277 | −0.19938 | 0.027689 | −0.26501 | MSL | 0.069122 | IM | −0.02541 |
| K75958547 | −0.25647 | −0.20299 | 0.024936 | −0.40468 | MSL | −0.04476 | M | −0.0837 |
| K84868168 | −0.17592 | −0.14067 | 0.022248 | −0.18424 | MSL | −0.0744 | M | 0.006092 |
| K13169950 | −0.18811 | −0.2034 | 0.025243 | −0.26133 | M | 0.093556 | M | −0.00012 |
| K84868168 | −0.32394 | −0.31993 | 0.000368 | −0.43505 | MSL | −0.0744 | M | 0.006092 |
| A19736161 | −0.1616 | −0.15834 | 0.012533 | −0.19919 | IM | −0.07634 | IM | −0.11235 |
| K79821389 | −0.18602 | −0.25324 | 3.25E−05 | −0.20379 | IM | −0.11776 | IM | −0.06417 |
| K67578145 | −0.15832 | −0.19925 | 0.006548 | −0.20293 | M | 0.110094 | M | 0.15809 |
| K19333160 | −0.19525 | −0.15882 | 0.008569 | −0.21333 | MSL | 0.046328 | MSL | 0.081223 |
| K67578145 | −0.20015 | −0.14902 | 0.016814 | −0.20635 | M | 0.110094 | M | 0.15809 |
| K94455792 | −0.15061 | −0.16395 | 0.006632 | −0.23104 | M | 0.178329 | M | 0.163484 |
| K19687926 | −0.18741 | −0.15157 | 0.012005 | −0.16446 | IM | −0.08369 | IM | −0.0302 |
| K42805893 | −0.29476 | −0.18764 | 0.001776 | −0.17527 | IM | −0.06781 | IM | 0.075302 |
| K46386702 | −0.2314 | −0.14635 | 0.015515 | −0.21657 | IM | −0.05992 | IM | −0.03109 |
| K05804044 | −0.26232 | −0.15815 | 0.008857 | −0.1733 | M | 0.190801 | M | 0.324717 |
| K13394247 | −0.17284 | −0.15372 | 0.033271 | −0.2128 | M | 0.059801 | MSL | −0.04236 |
| K12040459 | −0.21449 | −0.14352 | 0.024367 | −0.17868 | MSL | 0.048117 | M | 0.003574 |
| A12230535 | −0.21153 | −0.18337 | 0.01155 | −0.24656 | M | 0.160633 | M | 0.150256 |
| K81332461 | −0.35213 | −0.12972 | 0.036948 | −0.17447 | IM | −0.07504 | IM | −0.00506 |
| K31866293 | −0.29319 | −0.12524 | 0.048374 | −0.20673 | M | 0.084888 | M | 0.20887 |
| K56981171 | −0.18505 | −0.20637 | 0.004081 | −0.18953 | IM | −0.17279 | IM | 0.040238 |
| A56085258 | −0.23289 | −0.20467 | 0.000653 | −0.23575 | M | 0.095349 | M | 0.14417 |
| K26818574 | −0.26243 | −0.20577 | 0.000624 | −0.17753 | M | 0.083765 | M | 0.246345 |
| K12040459 | −0.18863 | −0.12532 | 0.038525 | −0.2028 | MSL | 0.048117 | M | 0.003574 |
| A56085258 | −0.27828 | −0.16993 | 0.004793 | −0.2418 | M | 0.095349 | M | 0.14417 |
| K05804044 | −0.26462 | −0.14752 | 0.014886 | −0.19098 | M | 0.190801 | M | 0.324717 |
| K31866293 | −0.24065 | −0.14213 | 0.019017 | −0.26994 | M | 0.084888 | M | 0.20887 |
| K13183738 | −0.3033 | −0.20342 | 0.000722 | −0.23393 | M | 0.215745 | M | 0.139961 |
| K29905972 | −0.21957 | −0.18554 | 0.002205 | −0.24346 | M | 0.081599 | M | 0.004812 |
| K47150025 | −0.27588 | −0.15532 | 0.010168 | −0.23856 | M | 0.025124 | M | 0.037321 |
| K77791657 | −0.16247 | −0.20043 | 0.00083 | −0.35165 | IM | −0.1309 | IM | −0.05245 |
| K16180792 | −0.22066 | −0.18412 | 0.002573 | −0.27993 | M | 0.148517 | M | 0.090307 |
| K31866293 | −0.26144 | −0.16506 | 0.020778 | −0.26705 | M | 0.084888 | M | 0.20887 |
| K19687926 | −0.21639 | −0.13638 | 0.023963 | −0.1645 | IM | −0.08369 | IM | −0.0302 |
| K37561857 | −0.16647 | −0.18834 | 0.039393 | −0.31546 | M | 0.178243 | MSL | 0.169947 |
| K93123848 | −0.17363 | −0.25722 | 0.000263 | −0.26581 | M | 0.101093 | M | 0.191571 |
| A75975749 | −0.27465 | −0.18253 | 0.011277 | −0.26801 | M | 0.119134 | M | 0.165805 |
| K94455792 | −0.19148 | −0.2288 | 0.011249 | −0.27796 | M | 0.178329 | M | 0.163484 |

### Table 24A and 24B Key

MoA: Mechanism of action.
Associated Gene: Gene whose expression correlated with compound sensitivity across cell lines.
Associated Gene Family: Protein family in which that gene is found.
compound gene train corr: Correlation of gene expression with compound sensitivity in the training data. Strong negative correlation means that greater expression was linked to increased sensitivity to the compounds.
compound gene train pvalue: Measure of significance for this association.
compound gene test corr: Correlation in the test data.
compound gene dep train corr: Correlation of gene dependency with compound sensitivity in the training data set. A negative correlation means that the importance of the expression of that gene to cell health was linked to increased compound sensitivity.
compound gene dep train pvalue: Measure of significance for this association.
compound gene dep test corr: Correlation of gene dependency with compound sensitivity in the test data set.
TNBCType train: Classification of association of the compound sensitivity with the IM, MSL and M cell line classes in the training data set.
DTIO Corr train: Association of the compound sensitivity with the DetermaIO cell line classes in the training data set.
TNBCType test: Classification of the association of the compound sensitivity with the IM, MSL and M cell line classes in the test data set.
DTIO Corr test: Association of the compound sensitivity with the DetermaIO cell line classes in the test data set.

TABLE 25
Unique compounds identified through assessment of compound:gene
and compound:gene:gene dependency correlations.

| | | | Present |
|---|---|---|---|
| | | Present | in com- |
| BROAD | | in com- | pound:gene:gene |
| com- | | pound:gene | dependency |
| pound ID | Compound Name | correlations | correlations |
| A00842753 | RGFP966 | | X |
| A12230535 | TAK-285 | | X |
| A19736161 | benzethonium | | X |
| A19777893 | oleuropein | X | |
| A27883417 | nutlin-3 | X | |
| A56085258 | ondansetron | X | X |
| A75975749 | menadione-bisulfite | X | X |
| K01507359 | alexidine | X | |
| K02113016 | gefitinib | | X |
| K03289018 | BIBX-1382 | | X |
| K03765900 | LGX818 | X | |
| K04568635 | bafetinib | X | |
| K05804044 | rifampin | X | X |
| K07106112 | olaparib | X | |
| K08542803 | dasatinib | X | X |
| K09416995 | CCT137690 | X | |
| K09951645 | chlorhexidine | X | X |
| K12040459 | rubitecan | | X |
| K13044802 | GDC-0879 | | X |
| K13060017 | XL-647 | X | |
| K13169950 | PR-619 | X | X |
| K13183738 | octenidine | X | X |
| K13394247 | oxiperomide | | X |
| K16180792 | AZ-628 | X | X |
| K17203476 | dihydroartemisinin | | X |
| K19333160 | BMS-599626 | X | X |
| K19540840 | gambogic-acid | X | |
| K19687926 | BMS-690514 | | X |
| K20285085 | UNC0642 | | X |
| K22134346 | lovastatin | X | |
| K23190681 | dabrafenib | X | |
| K23925186 | AT7867 | | |
| K25970317 | artesunate | X | |
| K26603252 | ciclopirox | X | |
| K26818574 | UNC0631 | X | X |
| K28061410 | NSC-3852 | X | |
| K28824103 | pentamidine | X | |
| K29905972 | RAF265 | | X |
| K30159788 | radafaxine | X | |
| K30933884 | bis(maltolato)oxova- | X | |
| | nadium(IV) | | |
| K31698212 | LY2874455 | X | |
| K31866293 | RKI-1447 | | X |
| K33882852 | saracatinib | X | |
| K37561857 | lapatinib | X | X |
| K39974922 | thiomersal | | X |
| K40109029 | R406 | X | |
| K42805893 | 3-deazaneplanocin-A | | X |
| K46386702 | simvastatin | X | X |
| K47150025 | mevastatin | | X |
| K49294207 | AV-412 | X | |
| K49328571 | oridonin | X | |
| K52256627 | resibufogenin | X | |
| K54395039 | PD-153035 | X | |
| K54634444 | BIX-01294 | X | |
| K54955827 | atorvastatin | | X |
| K56981171 | erdafitinib | | X |
| K58529924 | beta-lapachone | X | |
| K60130390 | genipin | X | |
| K60443845 | axitinib | X | |
| K61443650 | niraparib | X | |
| K61688984 | baicalein | | X |
| K62200014 | RSV604 | X | |
| K62213621 | UNBS-5162 | X | |
| K64052750 | icotinib | X | |
| K67578145 | TAK-632 | X | X |
| K69726342 | ZK-93423 | X | |
| K70914287 | zardaverine | X | |
| K72327355 | chlormidazole | | X |
| K72723676 | lenvatinib | X | |
| K75958547 | anagrelide | | X |
| K76239644 | SB-505124 | X | |
| K77791657 | ICG-001 | | X |
| K78096648 | osimertinib | X | |
| K79821389 | pitavastatin | | X |
| K80343549 | ARRY-334543 | X | |
| K81332461 | maxacalcitol | | X |
| K84868168 | selumetinib | X | X |
| K93123848 | KI-8751 | X | X |
| K94441233 | ONC201 | X | X |
| K94455792 | BIBU-1361 | X | X |

TABLE 26
Unique genes identified through assessment of compound:gene
and compound:gene:gene dependency correlations.

| | | | Present in |
|---|---|---|---|
| | | | compound: |
| | | Present in | gene:gene |
| | | compound:gene | dependency |
| | Gene | correlations | correlations |
| | A2M | X | |
| | ABCA8 | X | |
| | ABI2 | X | |
| | ABL1 | | X |
| | ACACB | X | |
| | ACTR3B | X | |
| | ADAM11 | X | |
| | ADAM23 | X | |
| | ADAM8 | X | |
| | ADAMTS1 | X | |
| | ADAMTS9 | X | |
| ADAMTSL1 | | | X |
| | ADGRB2 | X | |
| | AEBP1 | X | |
| | ALOX5 | X | |
| | AMOTL1 | X | |
| | ANGPTL1 | X | |
| | ANGPTL7 | X | |
| | ANKRD33B | X | |
| | ANKRD44 | X | |
| | ANKRD65 | X | |
| | ANTXR1 | X | |
| | ANXA6 | X | |
| | APOD | X | |
| | APOL1 | X | |
| | APOLD1 | X | |
| | ARHGAP30 | X | |
| | ARHGAP31 | X | |
| | ARHGDIB | X | |
| | ARHGEF4 | X | |
| | ARL10 | X | |
| | ARMCX1 | X | |
| | ASB2 | X | |
| | ATOH8 | X | |
| | ATP8B2 | X | |
| | B4GALNT1 | X | |
| | BCL2A1 | X | X |
| | BCL2L14 | X | |
| | BEND6 | X | |
| | BIRC3 | X | |
| | C15orf48 | X | |
| | C3AR1 | X | |
| | C3orf18 | X | |
| | C3orf70 | X | |
| | CACNA1G | | X |
| | CALD1 | X | |
| | CAMK4 | X | |
| | CAND2 | X | |
| | CATSPER1 | X | |
| | CAVIN1 | X | X |
| | CBX1 | X | |
| | CCDC136 | X | |
| | CCDC80 | X | |
| | CCN2 | X | |
| | CD96 | X | |
| | CDH19 | X | |
| | CERKL | X | |
| | CERS1 | X | |
| | CHD3 | X | |
| | CHL1 | X | |
| | CHST10 | X | |
| | CHST11 | X | |
| | CIITA | X | |
| | CLEC12A | X | |
| | CLEC1A | | X |
| | CLEC4E | X | |
| | CLEC7A | X | |
| | CLIC5 | X | |
| | CLIP4 | X | |
| | CMTM4 | X | |
| | CMTM5 | X | |
| | CNRIP1 | X | |
| | CNTNAP1 | X | X |
| | COL13A1 | X | |
| | COL17A1 | X | |
| | COL19A1 | X | |
| | COL4A2 | X | |
| | COL9A3 | X | |
| | CORO2B | X | |
| | CPQ | X | |
| | CRMP1 | | X |
| | CSPG4 | X | |
| | CST7 | X | |
| | CTLA4 | X | |
| | CUBN | X | |
| | CXCL16 | X | |
| | CYBRD1 | X | |
| | CYGB | X | |
| | CYP2U1 | X | |
| | CYP4B1 | X | |
| | DAAM2 | X | |
| | DACT3 | X | |
| | DAPP1 | X | |
| | DCLK2 | X | X |
| | DENND1C | X | |
| | DENND2D | X | |
| | DIRAS1 | X | |
| | DKK2 | X | |
| | DLC1 | X | |
| | DLG4 | X | |
| | DLX2 | X | |
| | DLX5 | X | |
| | DLX6 | X | |
| | DRAM1 | X | |
| | DSG3 | X | |
| | DSTYK | X | |
| | DVL2 | X | |
| | EBF1 | | X |
| | EBI3 | | X |
| | EDNRB | X | X |
| | EMILIN3 | X | |
| | ENOX1 | X | |
| | EPHA3 | X | |
| | EPHB3 | | X |
| | EPHB3 | | X |
| | EPHB3 | | X |
| | EPSTI1 | | X |
| | EVC | X | |
| | EVC2 | | X |
| | EVI2A | X | |
| | EXTL2 | X | |
| | FAM171A2 | X | |
| | FAM180B | X | |
| | FAM78A | X | |
| | FAT4 | | X |
| | FBLN1 | X | |
| | FBN1 | X | X |
| | FBXL7 | X | |
| | FBXO17 | | X |
| | FCER1G | X | |
| | FCGR2A | X | |
| | FCGR2B | X | |
| | FCMR | X | |
| | FCRLA | X | |
| | FERMT2 | X | |
| | FGD1 | X | |
| | FGD3 | X | |
| | FGFR1 | X | |
| | FGL2 | X | |
| | FHL1 | X | X |
| | FKBP7 | X | |
| | FLT1 | X | X |
| | FMNL3 | X | |
| | FN1 | X | |
| | FOXO3B | X | |
| | FRMD6 | X | |
| | FYN | X | |
| | GALNT17 | X | |
| | GAS7 | X | |
| | GASK1B | X | |
| | GDNF | X | |
| | GHR | X | |
| | GLI1 | X | |
| | GLIPR2 | X | |
| | GNG2 | X | |
| | GNG7 | X | |
| | GPC3 | X | |
| | GPC6 | X | |
| | GPR161 | X | |
| | GPR162 | X | |
| | GPR55 | X | |
| | GPSM1 | X | |
| | GPSM3 | X | |
| | GSN | X | |
| | GUCY1A2 | X | |
| | GYPC | X | |
| | HAND2 | X | |
| | HEG1 | X | |
| | HGF | X | |
| | HMCN1 | X | |
| | HPS5 | X | |
| | HSH2D | X | |
| | HSPG2 | | X |
| | HTRA1 | X | |
| | IFFO1 | X | |
| | IGFBP7 | X | |
| | IL12RB2 | X | |
| | IL15RA | X | |
| | IL16 | X | |
| | IL23A | X | |
| | IL24 | X | |
| | IL32 | X | |
| | IL4I1 | | X |
| | IRF4 | X | |
| | ISG15 | X | |
| | ITGA10 | X | |
| | ITGA4 | X | |
| | ITGA9 | X | |
| | ITGB3 | X | |
| | ITGB4 | X | X |
| | ITIH5 | X | |
| | ITK | X | |
| | JAM3 | X | |
| | JAZF1 | X | |
| | KANK2 | X | |
| | KBTBD6 | X | |
| | KCNAB1 | X | |
| | KCNH2 | X | |
| | KCNJ10 | X | |
| | KCNJ4 | X | |
| | KDR | X | |
| | KIF5A | X | |
| | KIRREL1 | X | |
| | KLF8 | X | |
| | KLHL29 | X | |
| | KRBA1 | X | |
| | KRT14 | X | |
| | KRT16 | X | |
| | KRT17 | X | X |
| | KRT5 | X | |
| | LAMA4 | X | |
| | LARP6 | X | |
| | LAYN | X | |
| | LCP2 | X | X |
| | LGALS9 | X | |
| | LIMD2 | | X |
| | LIX1L | X | |
| | LPAR4 | X | |
| | LPAR5 | X | |
| | LPXN | X | X |
| | LRRC8C | X | |
| | LSAMP | X | |
| | LTB | X | |
| | LTBP2 | | X |
| | LUM | X | |
| | LYL1 | X | |
| | LZTS1 | X | |
| | LZTS2 | X | |
| | MAP1B | X | |
| | MAP3K3 | X | |
| | MAPK10 | X | |
| | MCAM | X | |
| | MCC | X | |
| | MCOLN2 | X | |
| | MEF2C | X | |
| | MEX3A | X | |
| | MEX3B | X | |
| | MFGE8 | X | |
| | MFNG | X | |
| | MIA | X | |
| | MICB | | X |
| | MICU3 | X | |
| | MLLT1 | X | |
| | MMP16 | X | |
| | MOXD1 | X | |
| | MPDZ | X | |
| | MPP2 | X | |
| | MRAS | X | |
| | MRC2 | X | |
| | MSI1 | X | |
| | MSRB3 | X | |
| | MYH10 | X | |
| | MYLK | X | |
| | NCKAP5L | X | |
| | NES | X | |
| | NEXMIF | X | |
| | NFASC | | X |
| | NFATC2 | X | |
| | NGFR | X | X |
| | NLGN1 | X | |
| | NLGN2 | X | |
| | NMI | X | |
| | NPL | X | |
| | NREP | X | X |
| | NRP2 | X | |
| | NRROS | X | X |
| | NTN4 | X | |
| | NUDT11 | X | |
| | NYNRIN | X | |
| | OBSL1 | X | |
| | P2RX7 | X | |
| | PALM | X | |
| | PARD6G | X | |
| | PCDHB7 | X | |
| | PDE1C | X | |
| | PDE7B | X | |
| | PDGFRB | X | |
| | PDZD4 | X | |
| | PDZRN3 | X | |
| | PEAK1 | X | |
| | PHC1 | X | |
| | PHYHIP | X | |
| | PIANP | X | |
| | PIK3CD | X | |
| | PIP4K2B | X | |
| | PKNOX2 | X | |
| | PLAAT4 | X | |
| | PLEKHO1 | X | |
| | PLEKHO2 | X | |
| | PLP1 | X | |
| | PLPP7 | X | |
| | PLXNB3 | X | X |
| | PLXNC1 | X | |
| | PMP2 | X | |
| | POLR2A | X | X |
| | PRICKLE2 | | X |
| | PRKD1 | X | |
| | PRTG | X | |
| | PSMA3 | | X |
| | PSME1 | X | |
| | PTAFR | X | |
| | PTCRA | X | |
| | PTK7 | X | |
| | PTN | X | |
| | PTPN14 | | X |
| | PTPN14 | | X |
| | PTPN6 | X | |
| | PTPRG | | X |
| | PTPRM | X | |
| | PTPRZ1 | X | |
| | PYGO1 | X | |
| | QKI | X | |
| | RAC2 | X | |
| | RASGEF1B | X | |
| | RASL10B | X | |
| | RASSF4 | X | X |
| | RASSF5 | X | |
| | RASSF8 | X | |
| | RBPMS2 | X | |
| | RCN2 | X | |
| | RCOR2 | X | X |
| | RECK | X | |
| | REEP2 | X | |
| | RENBP | X | |
| | RGS1 | X | |
| | RHOJ | X | |
| | RTL5 | X | |
| | RUNX3 | X | |
| | RUSC2 | X | |
| | S100A8 | X | |
| | SALL2 | X | |
| | SCARA5 | X | |
| | SCARF2 | X | |
| | SCML4 | X | |
| | SCRG1 | X | |
| | SGCA | X | |
| | SGCD | X | |
| | SH2D1B | | X |
| | SH2D1B | | X |
| | SH3BP1 | X | |
| SH3PXD2A | | | X |
| | SH3PXD2B | X | |
| | SHANK1 | X | |
| | SHC4 | X | |
| | SHISA4 | X | |
| | SHROOM4 | X | |
| | SIRPB2 | X | |
| | SLC35F1 | X | |
| | SLC6A12 | X | |
| | SMIM10 | X | |
| | SMO | X | |
| | SNX10 | X | |
| | SORBS1 | X | |
| | SORCS1 | X | |
| | SORCS2 | X | |
| | SOX5 | X | |
| | SPARC | X | |
| | SPART | X | |
| | SPTLC2 | X | |
| | SRGN | X | |
| | SRPX | X | |
| | ST3GAL2 | X | |
| | ST3GAL5 | X | |
| | ST6GALNAC3 | X | |
| | ST8SIA1 | X | |
| | STARD9 | X | |
| | STK10 | X | |
| | STK32B | X | |
| | SYDE1 | X | |
| | SYNM | X | X |
| | SYPL2 | X | |
| | SYTL3 | X | |
| | TACC1 | | X |
| | TAMALIN | X | |
| | TEAD3 | X | |
| | TIMP2 | X | |
| | TIMP3 | X | |
| | TLCD5 | X | |
| | TMEM229B | X | |
| | TMEM255A | X | |
| | TMPO | X | |
| | TMPRSS3 | | X |
| | TMTC1 | X | |
| | TNF | X | |
| | TNFAIP3 | X | |
| | TNFAIP8 | X | |
| | TNFRSF14 | X | |
| | TNFSF10 | X | |
| TNFSF13B | | | X |
| | TRAF3IP3 | X | |
| | TRPC1 | X | |
| | TRPV2 | X | |
| | TTC28 | X | |
| | TUB | X | |
| | TUBA1A | X | |
| | UNC13D | X | |
| | USP22 | X | |
| | VASH1 | X | |
| | VAV1 | X | |
| | VIM | X | |
| | VIPR1 | X | |
| | VSIR | X | |
| | WARS1 | X | |
| | WFDC1 | X | |
| | WIPF1 | X | |
| | WWTR1 | X | |
| | XCL1 | X | |
| | ZC3H12B | X | |
| | ZCCHC24 | X | |
| | ZEB1 | X | |
| | ZEB2 | X | |
| | ZMYND15 | X | |
| | ZNF101 | X | |

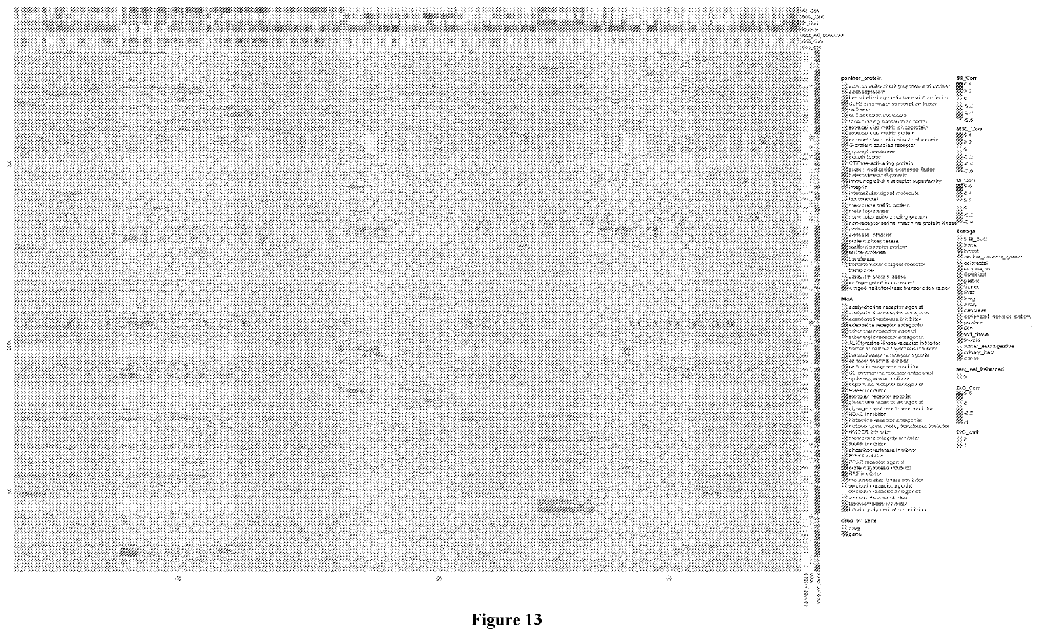

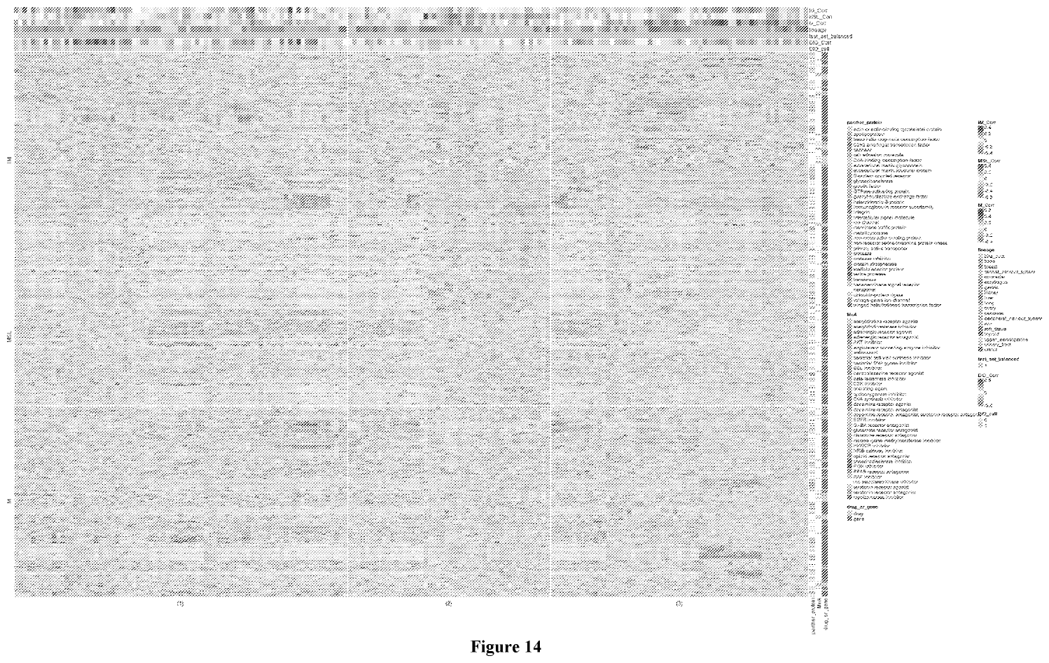

Relationships between gene expression and compound sensitivity in relation to subtypes can be visualized and explored via clustering. Using the gene list and selected compounds as described above, both datasets can be clustered and plotted as a heatmap. Clustering using the training set of cell lines shows genes and compounds with potentially related pathways and mechanisms of action co-clustering (FIG. 13). Mechanisms of action appear to have relationships with subtypes (Table 23), which were also observed in the test set of cell lines (FIG. 14).

These results were also validated through application of methods described herein to datasets obtained from the Cancer Therapeutics Response Portal (CTRP), which provided data from a screen of cancer cell lines for small-molecule sensitivity (Seashore et al., Cancer Discov. 2015 November; 5(11): 1210-23). As described for the PRISM screen, correlation of compound sensitivity across the cell lines to the IM, MSL and M subtype was determined, as well as measurements of significance (p values and q values). q values were determined as described by John D. Storey, Robert Tibshirani, Statistical significance for genomewide studies, Proceedings of the National Academy of Sciences August 2003, 100 (16) 9440-9445 using the q value R package. Compound correlations where the correlation was negative, indicating sensitivity to the compound, and with a q value less than 0.05 were assessed. From this list of compounds, 83% produced comparable results in both the PRISM and CTRP assays, suggesting a high degree of confidence in observed correlations.

Similarly, the results from the PRISM screen which had a q value less than 0.05 and were also screened in the CTRP assay (N=10) were assessed. 90% of these gave comparable results in both assays. Compounds classified as inhibitors of EGFR were identified as highly significant in both the CTRP and PRISM screens. Tumors of the IM class were also identified as especially sensitive to certain EGFR inhibitors (e.g. lapatinib, canertinib, afatinib). Interestingly, several drugs in this class have shown to have immunomodulatory effects during cancer treatment (Griguolo, et al., J. Immunotherapy Cancer 7, 90 (2019); Tu et al., Cancer Res. 2021 Jun. 15; 81(12): 3270-3282). suggests that patients with a tumor classified as IM by TNBC Type (the 101 gene signature) might be candidates for this class of drug.

Among other things, the present example demonstrates that compounds may be classified by association with genes of particular subtypes (IM, MSL, M) in order to inform selection of one or more therapies. In some embodiments, a tumor sample (e.g., obtained through liquid biopsy, tissue biopsy, etc.) may be assessed to determine expression level of one or more genes or miRNAs as described herein. In some embodiments, a tumor sample may be assessed for DetermaIO scoring. In some embodiments, selection of treatment with one or more compounds may be based upon DetermaIO score. For example, in some embodiments, one or more compound treatments may be selected due to increased sensitivity of a tumor sample to such treatment(s) depending on DetermaIO score. In some embodiments, one or more compound treatments may be selected due to increased sensitivity of a tumor sample to such treatment(s) depending expression levels of genes and/or miRNAs classified in a particular subtype (e.g., IM, M, MSL).

In some embodiments, the present example demonstrates that certain compounds (e.g., anti-VEGF compounds, vitamin D receptor inhibitors) may associate with the MSL subtype. In some embodiments, the present example demonstrates that certain compounds (e.g., CSF1R inhibitors) may associate with the M subtype. In some embodiments, the present example demonstrates that certain compounds (e.g., CD40 agonists) may associate with the IM subtype.

### Example 16: Assessment of Tumor Immune Signatures

The present Example, among other things, demonstrates that classifications provided herein can be correlated with tumor immune infiltrate types and may be utilized and/or included in assessments of subjects and/or their immune statuses and/or responsiveness to therapy.

Gene expression data sets from tumors in eighteen types (lung squamous cell, lung adenocarcinoma, breast, ovarian, kidney clear cell, head and neck, prostate, melanoma, colon, bladder, pancreas, kidney papillary, sarcoma, rectal, B cell lymphoma, kidney chromophobe, esophageal and stomach cancers) were downloaded from the US NIH Genomic Data Commons Portal (https://portal.gdc.cancer.gov/). Each tumor's gene expression data was processed identically. Experiment data downloaded from the NIH data repository was converted to a DGE list using the R (version 4.1.1) function SE2DGEList in the edgeR package. Duplicate genes were reduced to a single gene by selecting the gene with the maximum standard deviation across all samples in the data set. HUGO Gene Nomenclature Committee (HGNC) names for each gene were added using the biomarRt package. Genes without a HGNC designation were removed from the analysis. Using TMM normalization in the EdgeR package, normalization factors were calculated, applied, and log 2 transformed counts per million (cpm) were determined for each gene for each sample. The distribution of cpm and the standard deviation of genes across samples was plotted, and cutoffs determined to remove very low expressing genes. Samples were then renormalized using TMM after these genes were removed. Potential confounding effects due to the institution from which a tumor sample was acquired was addressed by removing batch effect based on the tissue source site using the EdgeR package. The normalized and batch corrected gene expression data sets were then combined into a 2/3 training, 1/3 test set with tumors balanced for tumor type between the two sets, without shared institutions between the two. This results in 4696 tumor samples in the training set, and 2195 samples in the test. This gene expression data set is referred to herein as TCGA18.

One set of immune cell signatures were created from defined mouse immune cell populations. Gene expression data from the ImmGen public consortium (www.immgen.org) was downloaded. These data are derived from defined and purified mouse cells using the ImmGen ULI GSE109125 data set. Gene names were translated into human homologs, and when duplicate genes were present, those with maximum standard deviation across all lines were selected. This list of genes was limited to those that were present in a list of 937 genes derived from an analysis of lung squamous, melanoma and sarcoma tumor gene expression data sets to allow for maximum diversity between clusters defined by the 101 gene signatures, resulting in 740 genes. A data set was created using these 740 genes and the normalized and batch corrected data from TCGA18. Using these data, gene expression of tumor samples were correlated with gene expression of the ImmGen cells. Principal components were calculated and plotted for these correlations. The first component largely defined a tumor vs purified cell signature, so was removed, and a new correlation value for all samples was determined, and the average of the correlations with the ImmGen samples for each class of ImmGen sample was calculated. The average expression of genes for each cluster was calculated as the “ImmGen corr subPC1” signature. The average expression of these signatures for all assayed cells is summarized by subtype (e.g., IM, MSL, M) and tissue of origin (Table 27A).

TABLE 27A
Average signature expression by subtype (IM, M, MSL) and tissue of origin.

| | | | | | ImmGen | | |
|---|---|---|---|---|---|---|---|
| | | ImmGen | | ImSig | corr | community | |
| | ImSig | corr | | score. | subPC1 | Macro- | ImSig |
| | score. | subPC1 | community | Macro- | macro- | phage | score. |
| | B cells | B cell | B cell | phages | phage | M1 M2 | T cells |
| Bcell lymphoma | | | | | | | |
| IM | 13.99 | 0.33 | 1.85 | 91.59 | 0.33 | 4.56 | 16.41 |
| bladder | | | | | | | |
| IM | 14.90 | 0.26 | 4.21 | 132.09 | 0.33 | 2.93 | 22.00 |
| M | 8.66 | 0.27 | 3.19 | 52.38 | 0.31 | 1.07 | 9.80 |
| MSL | 24.76 | 0.28 | 3.12 | 89.64 | 0.31 | 2.35 | 19.05 |
| breast | | | | | | | |
| IM | 21.56 | 0.28 | 3.25 | 146.55 | 0.33 | 3.90 | 31.39 |
| M | 11.21 | 0.28 | 2.44 | 64.94 | 0.32 | 2.37 | 10.20 |
| MSL | 13.07 | 0.28 | 2.27 | 88.10 | 0.32 | 2.93 | 14.94 |
| colon | | | | | | | |
| IM | 13.95 | 0.27 | 3.28 | 98.69 | 0.32 | 2.38 | 17.38 |
| M | 11.64 | 0.27 | 2.49 | 67.51 | 0.31 | 1.41 | 12.22 |
| MSL | 19.29 | 0.29 | 2.69 | 104.69 | 0.33 | 2.67 | 20.14 |
| esophageal | | | | | | | |
| IM | 15.68 | 0.27 | 5.20 | 109.32 | 0.32 | 2.71 | 19.66 |
| M | 11.49 | 0.27 | 4.77 | 70.13 | 0.32 | 1.60 | 12.24 |
| MSL | 16.25 | 0.29 | 4.14 | 91.97 | 0.31 | 2.60 | 17.62 |
| Head and neck | | | | | | | |
| IM | 15.13 | 0.26 | 5.36 | 106.47 | 0.33 | 2.84 | 19.66 |
| M | 11.44 | 0.27 | 4.89 | 56.19 | 0.32 | 2.06 | 8.91 |
| MSL | 13.76 | 0.28 | 4.30 | 120.33 | 0.34 | 3.64 | 19.08 |
| kidchrome | | | | | | | |
| IM | 14.78 | 0.27 | 1.55 | 105.81 | 0.34 | 2.11 | 17.36 |
| M | 13.01 | 0.27 | 0.69 | 71.85 | 0.32 | 0.88 | 14.87 |
| MSL | 13.73 | 0.27 | 1.50 | 89.61 | 0.33 | 1.87 | 16.55 |
| kidclear | | | | | | | |
| IM | 14.82 | 0.26 | 2.40 | 126.93 | 0.34 | 3.83 | 21.97 |
| M | 11.21 | 0.27 | 1.75 | −5.30 | 0.30 | 1.69 | 0.43 |
| MSL | 13.00 | 0.27 | 2.07 | 46.14 | 0.32 | 3.17 | 9.37 |
| kidpap | | | | | | | |
| IM | 15.25 | 0.26 | 3.09 | 117.09 | 0.38 | 3.25 | 19.70 |
| M | 10.99 | 0.26 | 2.23 | 40.49 | 0.35 | 1.76 | 10.78 |
| MSL | 14.55 | 0.27 | 2.56 | 92.12 | 0.36 | 2.85 | 15.35 |
| lungadeno | | | | | | | |
| IM | 14.68 | 0.28 | 4.17 | 94.86 | 0.34 | 4.11 | 18.25 |
| M | 3.48 | 0.28 | 3.90 | −17.12 | 0.32 | 2.54 | 0.53 |
| MSL | 13.14 | 0.29 | 4.13 | 89.84 | 0.35 | 3.95 | 13.65 |
| lungsquamous | | | | | | | |
| IM | 15.25 | 0.28 | 4.51 | 95.92 | 0.34 | 3.50 | 18.36 |
| M | 8.30 | 0.28 | 4.13 | 38.77 | 0.33 | 2.36 | 6.46 |
| MSL | 17.18 | 0.28 | 4.77 | 158.41 | 0.35 | 4.03 | 23.28 |
| melanoma | | | | | | | |
| IM | 27.16 | 0.27 | 1.43 | 171.98 | 0.34 | 3.93 | 34.49 |
| M | 3.90 | 0.27 | 1.10 | 29.78 | 0.34 | 2.19 | 3.17 |
| MSL | 24.01 | 0.28 | 0.81 | 154.56 | 0.33 | 3.55 | 24.64 |
| ovarian | | | | | | | |
| IM | 14.27 | 0.27 | 3.22 | 126.55 | 0.34 | 2.72 | 18.61 |
| M | 13.44 | 0.28 | 2.54 | 54.03 | 0.31 | 1.73 | 13.78 |
| MSL | 18.81 | 0.28 | 3.20 | 117.54 | 0.33 | 3.37 | 22.76 |
| pancreas | | | | | | | |
| IM | 31.96 | 0.28 | 4.36 | 132.17 | 0.34 | 3.95 | 23.59 |
| M | −3.95 | 0.27 | 3.96 | 62.92 | 0.34 | 2.91 | 6.69 |
| MSL | 18.73 | 0.29 | 4.20 | 98.00 | 0.33 | 3.67 | 19.23 |
| prostate | | | | | | | |
| IM | 22.82 | 0.26 | 1.89 | 134.39 | 0.30 | 2.47 | 28.74 |
| M | 12.38 | 0.26 | 0.75 | 80.21 | 0.29 | 0.72 | 13.37 |
| MSL | 14.15 | 0.26 | 1.24 | 93.14 | 0.29 | 1.53 | 16.80 |
| rectal | | | | | | | |
| IM | 13.91 | 0.27 | 3.48 | 93.99 | 0.33 | 2.50 | 16.82 |
| M | 12.27 | 0.27 | 3.01 | 78.64 | 0.32 | 1.88 | 13.72 |
| MSL | 16.94 | 0.28 | 3.18 | 102.20 | 0.33 | 2.76 | 18.96 |
| sarcoma | | | | | | | |
| IM | 20.94 | 0.26 | 1.33 | 260.61 | 0.36 | 4.72 | 45.67 |
| M | 10.79 | 0.26 | 0.20 | 9.32 | 0.32 | 2.00 | 5.84 |
| MSL | 15.92 | 0.26 | 1.02 | 143.06 | 0.35 | 3.86 | 21.62 |
| stomach | | | | | | | |
| IM | 13.46 | 0.27 | 4.40 | 115.29 | 0.33 | 3.36 | 19.48 |
| M | 2.29 | 0.27 | 3.94 | 41.90 | 0.32 | 2.22 | 6.69 |
| MSL | 22.74 | 0.28 | 3.98 | 84.03 | 0.31 | 3.15 | 17.62 |

| | ImmGen | ImmGen | ImmGen | | | |
|---|---|---|---|---|---|---|
| | corr | corr | corr | | | |
| | subPC1 | subPC1 | subPC1 | | | |
| | alpha- | gamma- | activated | community | community | community |
| | beta T | delta T | T | T cell 1 | T cell 2 | NKT |
| Bcell lymphoma | | | | | | |
| IM | 0.22 | 0.21 | 0.20 | 7.17 | −0.63 | 4.05 |
| bladder | | | | | | |
| IM | 0.25 | 0.25 | 0.24 | −0.15 | −0.54 | 2.08 |
| M | 0.26 | 0.25 | 0.23 | −1.15 | −0.68 | −0.37 |
| MSL | 0.26 | 0.25 | 0.23 | 1.00 | −0.59 | 1.08 |
| breast | | | | | | |
| IM | 0.25 | 0.25 | 0.23 | 1.59 | −0.51 | 3.13 |
| M | 0.25 | 0.25 | 0.22 | −0.55 | −0.78 | 0.50 |
| MSL | 0.25 | 0.25 | 0.23 | 0.25 | −0.77 | 1.37 |
| colon | | | | | | |
| IM | 0.26 | 0.25 | 0.23 | 0.10 | −0.18 | 1.49 |
| M | 0.26 | 0.25 | 0.23 | −0.91 | −0.22 | 0.02 |
| MSL | 0.25 | 0.25 | 0.22 | 1.68 | −0.19 | 1.61 |
| esophageal | | | | | | |
| IM | 0.25 | 0.25 | 0.23 | 0.83 | −0.53 | 1.97 |
| M | 0.26 | 0.25 | 0.23 | −0.56 | −0.82 | 0.15 |
| MSL | 0.25 | 0.25 | 0.23 | 1.31 | −0.54 | 1.22 |
| Head and neck | | | | | | |
| IM | 0.26 | 0.25 | 0.24 | 0.25 | −0.92 | 2.42 |
| M | 0.26 | 0.25 | 0.23 | −0.73 | −1.12 | 0.65 |
| MSL | 0.25 | 0.24 | 0.22 | 0.60 | −0.94 | 1.86 |
| kidchrome | | | | | | |
| IM | 0.24 | 0.25 | 0.22 | −1.31 | −0.41 | 0.12 |
| M | 0.25 | 0.25 | 0.22 | −2.14 | −0.70 | −1.38 |
| MSL | 0.25 | 0.25 | 0.23 | −1.36 | −0.63 | −0.04 |
| kidclear | | | | | | |
| IM | 0.24 | 0.25 | 0.23 | −0.63 | 0.22 | 2.55 |
| M | 0.25 | 0.25 | 0.23 | −1.69 | −0.68 | −0.58 |
| MSL | 0.25 | 0.25 | 0.23 | −0.91 | −0.38 | 1.07 |
| kidpap | | | | | | |
| IM | 0.23 | 0.24 | 0.21 | −0.64 | −0.33 | 1.57 |
| M | 0.24 | 0.24 | 0.21 | −1.88 | −0.40 | −0.25 |
| MSL | 0.24 | 0.24 | 0.21 | −0.54 | −0.60 | 0.71 |
| lungadeno | | | | | | |
| IM | 0.24 | 0.24 | 0.22 | 1.71 | 0.01 | 2.79 |
| M | 0.25 | 0.24 | 0.22 | −0.50 | −0.27 | 0.41 |
| MSL | 0.24 | 0.24 | 0.21 | 1.34 | 0.03 | 1.92 |
| lungsquamous | | | | | | |
| IM | 0.25 | 0.25 | 0.23 | 1.27 | −0.23 | 2.34 |
| M | 0.25 | 0.25 | 0.22 | 0.09 | −0.44 | 0.64 |
| MSL | 0.24 | 0.24 | 0.21 | 1.33 | 0.04 | 2.06 |
| melanoma | | | | | | |
| IM | 0.25 | 0.25 | 0.23 | 1.73 | −0.76 | 3.54 |
| M | 0.25 | 0.25 | 0.23 | −0.98 | −1.02 | 0.52 |
| MSL | 0.25 | 0.25 | 0.22 | 2.24 | −0.90 | 2.79 |
| ovarian | | | | | | |
| IM | 0.25 | 0.24 | 0.23 | −0.29 | −0.55 | 1.12 |
| M | 0.26 | 0.25 | 0.22 | −0.77 | −0.69 | −0.53 |
| MSL | 0.25 | 0.25 | 0.23 | 0.81 | −0.69 | 1.65 |
| pancreas | | | | | | |
| IM | 0.24 | 0.24 | 0.22 | 1.57 | 0.06 | 2.52 |
| M | 0.25 | 0.24 | 0.22 | −0.57 | −0.09 | 0.71 |
| MSL | 0.25 | 0.24 | 0.22 | 1.80 | 0.15 | 2.01 |
| prostate | | | | | | |
| IM | 0.27 | 0.27 | 0.24 | 0.79 | −0.61 | 2.39 |
| M | 0.27 | 0.27 | 0.24 | −1.69 | −0.93 | −0.26 |
| MSL | 0.27 | 0.27 | 0.24 | −0.57 | −0.92 | 0.68 |
| rectal | | | | | | |
| IM | 0.25 | 0.25 | 0.23 | −0.18 | −0.21 | 1.35 |
| M | 0.26 | 0.25 | 0.23 | −1.17 | −0.37 | 0.16 |
| MSL | 0.25 | 0.25 | 0.23 | 0.80 | −0.41 | 1.40 |
| sarcoma | | | | | | |
| IM | 0.24 | 0.24 | 0.23 | −0.22 | −0.48 | 3.43 |
| M | 0.25 | 0.25 | 0.23 | −2.09 | −0.74 | −0.49 |
| MSL | 0.25 | 0.24 | 0.23 | −0.80 | −0.62 | 1.40 |
| stomach | | | | | | |
| IM | 0.25 | 0.24 | 0.23 | 1.25 | −0.32 | 2.56 |
| M | 0.25 | 0.25 | 0.23 | −0.27 | −0.52 | 0.84 |
| MSL | 0.25 | 0.25 | 0.23 | 2.05 | −0.39 | 2.09 |

TABLE 27A
key

| Component | Class of signature | Cell type detected |
|---|---|---|
| ImSig score.B cells | ImSig | B cells |
| ImmGen corr subPC1 B | ImmGen vector with first principal | B cells |
| cell | component removed | |
| community B cell | Community detection signature | B cells |
| | derived from known markers | |
| ImSig score.Macrophages | ImSig | Macrophages |
| ImmGen corr subPC1 | ImmGen vector with first principal | Macrophages |
| macrophage | component removed | |
| community Macrophage | Community detection signature | Macrophages |
| M1 M2 | derived from known markers | |
| ImSig score.T cells | ImSig | T cells |
| ImmGen corr subPC1 | ImmGen vector with first principal | T cells, specifically gamma-delta |
| alpha-beta T | component removed | unactived T cells |
| ImmGen corr subPC1 | ImmGen vector with first principal | T cells, specifically alpha-beta |
| gamma-delta T | component removed | unactived T cells |
| ImmGen corr subPC1 | ImmGen vector with first principal | T cells, specifically actived T cells |
| activated T | component removed | |
| community T cell 1 | Community detection signature | T cells, specifically a mixed T cell |
| | derived from known markers | population |
| community T cell 2 | Community detection signature | T cells, specifically a mixed T cell |
| | derived from known markers | population |
| community NK T | Community detection signature | T cells, specifically a mixed T cell |
| | derived from known markers | population |

In addition to the ImmGen signatures, signatures were also created to identify sets of immune cells that could potentially infiltrate tumors together (e.g., natural killer cells and T cells). The signature used genes known to be markers for diverse immune cell types. A list of genes known to be differentially expressed in immune cell types was prepared comprising information from published sources as well additional genes of interest (Table 27B). Gene expression data for the training sets of tumor types breast, ovarian, bladder, lung adenocarcinoma and lung squamous cell carcinoma were each individually scaled. These scaled data sets were then clustered using fclust, with k=15. Clusters of gene identified by this process were limited to genes whose expression was three standard deviations above the mean for all genes. Clusters were only selected for further use if they contained at least ten genes. These selected clusters for all five tumors were combined and then used to create a network using the igraph package in R. Clusters of genes were defined by the cluster_louvain function in igraph. Genes defined by the community detection method were annotated by their cluster name (Table 28), which was derived from an examination of the immune markers that contributed to each cluster. The average expression of genes for each cluster was calculated as the community detection immune signature. The average expression of these signatures for all assayed cells is summarized by subtype (e.g., IM, MSL, M) and tissue of origin (Table 27A).

TABLE 27B
List of genes comprising those differentially expressed in
immune cell types in addition to other genes of interest.

| Gene | Cell Type |
|---|---|
| ABCB1 | Cancer stem cell |
| ABCB5 | Cancer stem cell |
| ABCG2 | Hematopoietic stem cell |
| ACTA2 | Fibroblast |
| ADGRE1 | Macrophage |
| AFP | Cancer stem cell |
| ALCAM | Mesenchymal stem cell |
| ALDH1A1 | Cancer stem cell |
| ANPEP | Mesenchymal stem cell |
| ARG1 | M2 macrophage |
| B3GAT1 | Natural killer cell |
| BLNK | B cell |
| BMI1 | Hematopoietic stem cell |
| CASP3 | Hematopoietic stem cell |
| CAV1 | Fibroblast |
| CCL1 | M2B macrophage |
| CCL11 | M1 macrophage |
| ccl15 | M1 macrophage |
| CCL17 | M2A macrophage |
| ccl17 | N2 neutrophil |
| CCL2 | M1 macrophage |
| ccl2 | N2 neutrophil |
| CCL22 | M2A macrophage |
| CCL24 | M2A macrophage |
| CCL3 | M1 macrophage |
| ccl3 | N1 neutrophil |
| ccl3 | N2 neutrophil |
| CCL4 | M1 macrophage |
| ccl4 | N2 neutrophil |
| CCL5 | M1 macrophage |
| CCL5 | M2D macrophage |
| CCL8 | M1 macrophage |
| ccl8 | N2 neutrophil |
| CCR1 | T helper1 (Th1) cell |
| CCR2 | M2C macrophage |
| CCR3 | Eosinophil |
| CCR4 | T helper2 (Th2) cell |
| CCR5 | T helper1 (Th1) cell |
| CCR8 | T helper2 (Th2) cell |
| CD14 | Macrophage |
| CD158 | Natural killer cell |
| CD160 | Natural killer cell |
| CD163 | M2 macrophage |
| CD163 | M2A macrophage |
| CD163 | M2C macrophage |
| CD19 | B cell |
| CD1A | Dendritic cell |
| CD1C | Dendritic cell |
| CD1D | Epithelial cell |
| CD2 | T cell |
| CD200R1 | M2A macrophage |
| CD209 | Dendritic cell |
| CD22 | B cell |
| CD24 | B cell |
| CD244 | Natural killer cell |
| CD28 | T cell |
| CD34 | Hematopoietic stem cell |
| CD37 | B cell |
| CD38 | M1 macrophage |
| CD3D | Natural killer cell |
| CD3E | Natural killer cell |
| CD3G | Natural killer cell |
| CD4 | Regulatory T (Treg) cell |
| CD40 | B cell |
| CD40LG | B cell |
| CD44 | Mesenchymal stem cell |
| CD47 | Cancer stem cell |
| CD48 | Hematopoietic stem cell |
| CD5 | T cell |
| CD5L | M2 macrophage |
| CD68 | M1 macrophage |
| CD69 | B cell |
| CD7 | T cell |
| CD70 | T Cell |
| CD74 | B cell |
| CD79A | B cell |
| CD79B | B cell |
| CD80 | M1 macrophage |
| CD83 | Dendritic cell |
| CD86 | M1 macrophage |
| CD86 | M2B macrophage |
| CD8A | CD8+ T cell |
| CD9 | Embryonic stem cell |
| CD93 | Hematopoietic stem cell |
| CDH1 | Embryonic stem cell |
| CDH1 | Epithelial cell |
| CDH5 | Endothelial cell |
| CEACAM6 | Cancer stem cell |
| CEACAM8 | Neutrophil |
| CHI3L1 | M2 macrophage |
| CHIT1 | M2 macrophage |
| Class | Dendritic cell |
| CLEC4C | Dendritic cell |
| CLEC7A | M2 macrophage |
| CR2 | B cell |
| CSF1R | Macrophage |
| CSF3R | Neutrophil |
| CTLA4 | Regulatory T (Treg) cell |
| CTNNB1 | Cancer stem cell |
| cxcl1 | N2 neutrophil |
| CXCL10 | M1 macrophage |
| CXCL10 | M2D macrophage |
| CXCL16 | M2D macrophage |
| cxcl16 | N2 neutrophil |
| cxcl2 | N2 neutrophil |
| CXCL8 | Neutrophil |
| cxcl8 | N2 neutrophil |
| CXCR1 | Neutrophil |
| CXCR2 | Neutrophil |
| CXCR3 | T helper1 (Th1) cell |
| CXCR4 | Hematopoietic stem cell |
| CXCR5 | B cell |
| DCLK1 | Cancer stem cell |
| DPP4 | Cancer stem cell |
| ECSCR | Endothelial cell |
| EGR2 | M2 macrophage |
| EMCN | Endothelial cell |
| ENG | Mesenchymal stem cell |
| ENTPD1 | Regulatory T (Treg) cell |
| EPCAM | Cancer stem cell |
| ERBB2 | Mesenchymal stem cell |
| EZH2 | Cancer stem cell |
| FAP | Fibroblast |
| fas | N1 neutrophil |
| FCER2 | B cell |
| FCGR1A | M1 macrophage |
| FCGR2A | M1 macrophage |
| FCGR2B | M1 macrophage |
| FCGR2C | M1 macrophage |
| FCGR3A | M1 macrophage |
| FCGR3A | Natural killer cell |
| FCGR3B | Natural killer cell |
| FLOT2 | Cancer stem cell |
| FLT1 | Endothelial cell |
| FLT3 | Hematopoietic stem cell |
| FLT4 | Endothelial cell |
| FOXP3 | Regulatory T (Treg) cell |
| FPR1 | Neutrophil |
| FPR2 | M1 macrophage |
| FUT4 | Neutrophil |
| FZD9 | Mesenchymal stem cell |
| GATA3 | T helper2 (Th2) cell |
| GFI1 | Hematopoietic stem cell |
| GLI1 | Cancer stem cell |
| GNG11 | Endothelial cell |
| GPR18 | M1 macrophage |
| HAVCR2 | T helperl (Th1) cell |
| HLA-ABC | Mesenchymal stem cell |
| HLA-DR | Mesenchymal stem cell |
| HLA-DRA | B cell |
| ICAM1 | Endothelial cell |
| icam1 | N1 neutrophil |
| ICAM2 | Endothelial cell |
| IFNG | T helper1 (Th1) cell |
| IFNGR1 | T helper1 (Th1) cell |
| II | Dendritic cell |
| IKZF2 | Regulatory T (Treg) cell |
| il10 | M2A macrophage |
| il10 | M2B macrophage |
| il10 | M2C macrophage |
| il10 | M2D macrophage |
| il12a | M1 macrophage |
| il12b | M2D macrophage |
| IL13 | T helper2 (Th2) cell |
| IL17A | T helper17 (Th17) cell |
| IL18R1 | T helper1 (Th1) cell |
| il1b | M1 macrophage |
| il1b | M2B macrophage |
| il1r1 | M1 macrophage |
| il1r2 | M2A macrophage |
| IL1RAP | Cancer stem cell |
| IL1RN | Macrophage |
| il23a | M1 macrophage |
| IL2RA | Regulatory T (Treg) cell |
| IL2RB | Natural killer cell |
| IL4 | T helper2 (Th2) cell |
| IL5 | T helper2 (Th2) cell |
| IL5RA | Eosinophil |
| IL6 | M1 macrophage |
| il6 | M2B macrophage |
| IL6R | Epithelial cell |
| IL6ST | Epithelial cell |
| ITGA1 | Mesenchymal stem cell |
| ITGA2 | Regulatory T (Treg) cell |
| ITGA4 | Mesenchymal stem cell |
| ITGA5 | Mesenchymal stem cell |
| ITGA6 | Mesenchymal stem cell |
| ITGAE | Epithelial cell |
| ITGAM | Neutrophil |
| ITGAV | Mesenchymal stem cell |
| ITGB1 | Mesenchymal stem cell |
| KLF4 | Cancer stem cell |
| KLRB1 | Natural killer cell |
| KLRC1 | Natural killer cell |
| KLRD1 | Natural killer cell |
| KLRK1 | Natural killer cell |
| KRT18 | Epithelial cell |
| KRT19 | Epithelial cell |
| KRT8 | Epithelial cell |
| LAG3 | Regulatory T (Treg) cell |
| LCN2 | Neutrophil |
| LGR5 | Cancer stem cell |
| LRRC32 | Regulatory T (Treg) cell |
| LRRC36 | T helper17 (Th17) cell |
| LTA | T helper1 (Th1) cell |
| LY9 | B cell |
| LYVE1 | Endothelial cell |
| LYZ | Macrophage |
| MCAM | Mesenchymal stem cell |
| MCL1 | Hematopoietic stem cell |
| MET | Cancer stem cell |
| MHC | Dendritic cell |
| MME | Fibroblast |
| MME | Mesenchymal stem cell |
| MNDA | Neutrophil |
| MPO | Neutrophil |
| MRC1 | M2 macrophage |
| mrc1 | M2A macrophage |
| MS4A1 | B cell |
| MS4A4A | M2 macrophage |
| MUC1 | Epithelial cell |
| MYB | Hematopoietic stem cell |
| MYC | M2 macrophage |
| MYD88 | Cancer stem cell |
| NANOG | Cancer stem cell |
| NCAM1 | Natural killer cell |
| NCR1 | Natural killer cell |
| NES | Cancer stem cell |
| NGFR | Mesenchymal stem cell |
| NKG7 | Natural killer cell |
| NOS2 | Epithelial cell |
| nos2 | M1 macrophage |
| NT5E | Mesenchymal stem cell |
| PAX5 | B cell |
| PDGFRA | Fibroblast |
| PDGFRB | Fibroblast |
| PDPN | Endothelial cell |
| PECAM1 | Mesenchymal stem cell |
| PLVAP | Endothelial cell |
| PODXL | Embryonic stem cell |
| POU2AF1 | B cell |
| POU2F2 | B cell |
| POU5F1 | Mesenchymal stem cell |
| PROCR | Endothelial cell |
| PROM1 | Mesenchymal stem cell |
| PTEN | Hematopoietic stem cell |
| PTGDR2 | T helper2 (Th2) cell |
| PTPRC | T cell |
| RETNLB | M2 macrophage |
| RORA | T helper17 (Th17) cell |
| S100A4 | Fibroblast |
| S100A8 | Neutrophil |
| S100A9 | Neutrophil |
| SELE | Endothelial cell |
| SELL | B cell |
| SELP | Endothelial cell |
| SIGLEC8 | Eosinophil |
| SLAMF1 | Hematopoietic stem cell |
| SOCS3 | M1 macrophage |
| SOX2 | Cancer stem cell |
| ST6GAL1 | B cell |
| STAB2 | Endothelial cell |
| STAT3 | T helper17 (Th17) cell |
| STAT5A | Hematopoietic stem cell |
| STAT5B | Hematopoietic stem cell |
| TBX21 | T helper1 (Th1) cell |
| TEK | Endothelial cell |
| TFRC | Mesenchymal stem cell |
| TGFB1 | Regulatory T (Treg) cell |
| TGFb1 | M2A macrophage |
| tgfb1 | M2C macrophage |
| tgfb1 | M2D macrophage |
| TGM2 | M2A macrophage |
| THBD | Endothelial cell |
| THY1 | Mesenchymal stem cell |
| TIGIT | Regulatory T (Treg) cell |
| TLR1 | M2C macrophage |
| TLR2 | M1 macrophage |
| TLR4 | M1 macrophage |
| TLR8 | M2C macrophage |
| TNF | T helper1 (Th1) cell |
| TNF | M1 macrophage |
| tnf | M2B macrophage |
| tnf | M2D macrophage |
| tnf | N1 neutrophil |
| TNFRSF13B | B cell |
| TNFRSF13C | B cell |
| TNFRSF18 | Regulatory T (Treg) cell |
| TNFRSF1A | Mesenchymal stem cell |
| TP63 | Cancer stem cell |
| TREM1 | Macrophage |
| VCAM1 | Mesenchymal stem cell |
| VEGFA | Endothelial cell |
| VEGFa | M2D macrophage |
| VWF | Endothelial cell |

TABLE 28
Genes defined by community detection method and annotated with cluster name.

| Gene | Cluster | Gene | Cluster | Gene | Cluster |
|---|---|---|---|---|---|
| PLVAP | endothelial | ENG | endothelial | TLR4 | macrophage |
| ECSCR | endothelial | MNDA | macrophage | CD14 | macrophage |
| GNG11 | endothelial | CD86 | macrophage | CD74 | macrophage |
| EMCN | endothelial | HAVCR2 | macrophage | HLA-DRA | macrophage |
| TEK | endothelial | FCGR2A | macrophage | IL2RA | macrophage |
| CD93 | endothelial | FCGR1A | macrophage | CD209 | macrophage |
| CD34 | endothelial | FCGR3A | macrophage | FPR1 | macrophage |
| CDH5 | endothelial | CD4 | macrophage | CD48 | NK_T |
| PECAM1 | endothelial | CCR1 | macrophage | CD8A | NK_T |
| VWF | endothelial | CD163 | macrophage | TBX21 | NK_T |
| FLT4 | endothelial | MS4A4A | macrophage | CXCR3 | NK_T |
| CAV1 | endothelial | CSF1R | macrophage | CD3D | NK_T |
| LYVE1 | endothelial | TLR8 | macrophage | CD2 | NK_T |
| LRRC32 | endothelial | ITGAM | macrophage | CD3E | NK_T |
| Gene | Cluster | Gene | Cluster | Gene | Cluster |
| CD3G | NK_T | KLRD1 | NK_T | TNFRSF13C | B_cell |
| CD5 | NK_T | NCR1 | NK_T | FCER2 | B_cell |
| LY9 | NK_T | CD7 | NK_T | POU2F2 | B_cell |
| SLAMF1 | NK_T | CCR5 | NK_T | CD22 | B_cell |
| TIGIT | NK_T | CTLA4 | NK_T | PTPRC | T_cell_plus |
| CCL4 | NK_T | CXCR5 | B_cell | LTA | T_cell_plus |
| KLRK1 | NK_T | MS4A1 | B_cell | CCR4 | T_cell_plus |
| GFI1 | NK_T | PAX5 | B_cell | CD28 | T_cell_plus |
| IL2RB | NK_T | CD79B | B_cell | CD40LG | T_cell_plus |
| NKG7 | NK_T | CD79A | B_cell | FLT3 | T_cell_plus |
| CCL5 | NK_T | POU2AF1 | B_cell | KLRB1 | T_cell_plus |
| LAG3 | NK_T | CD19 | B_cell | CCR2 | T_cell_plus |
| CXCL10 | NK_T | TNFRSF13B | B_cell | CD37 | T_cell_plus |
| IFNG | NK_T | CR2 | B_cell | | |

A previously defined set of immune signatures was also employed (Nirmal et al., Cancer Immunol Res. 2018 November; 6(11): 1388-1400). TCGA18 gene expression samples were processed as described above, but without a log transformation, and the imsig package in R used to classify samples. These signatures for all assayed cells are summarized by subtype (e.g. IM, MSL, M) and tissue of origin (Table 27A).

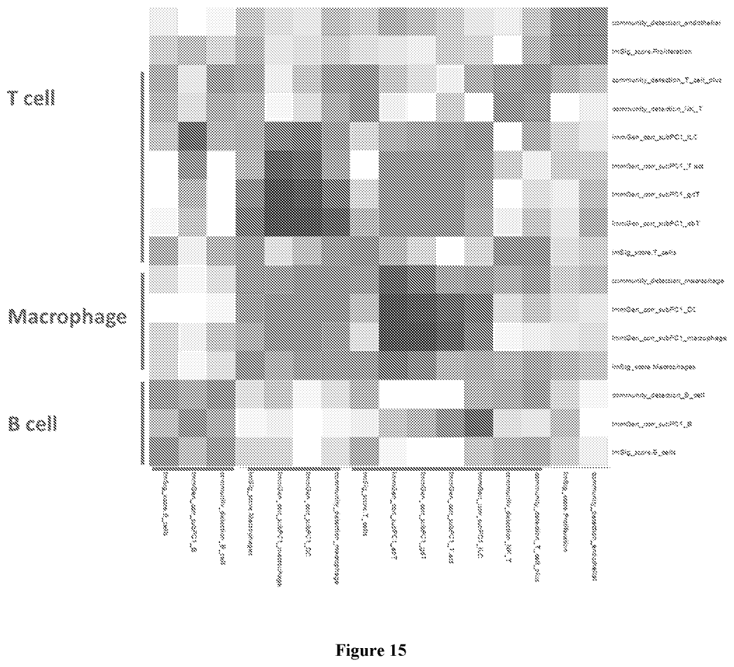

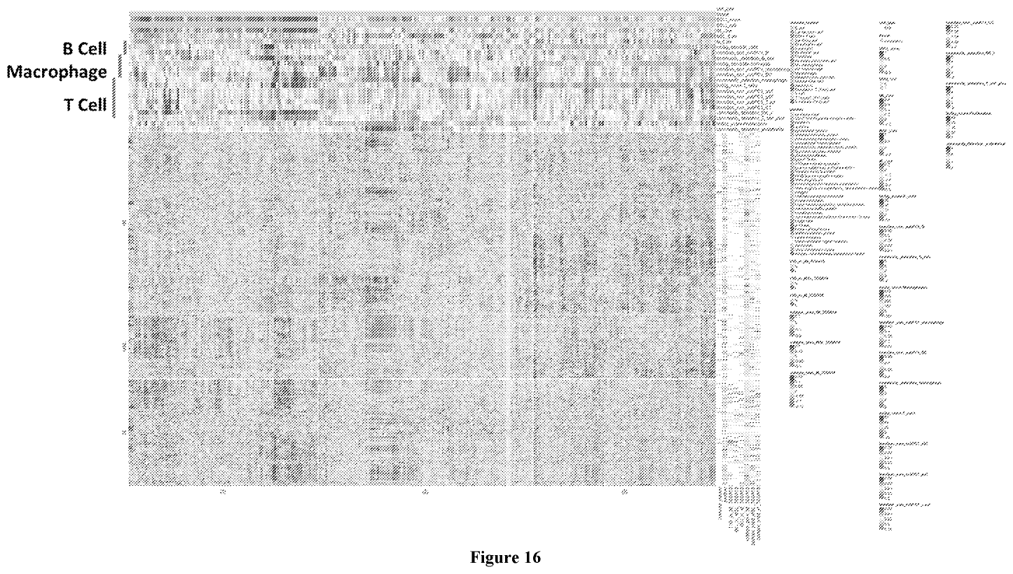

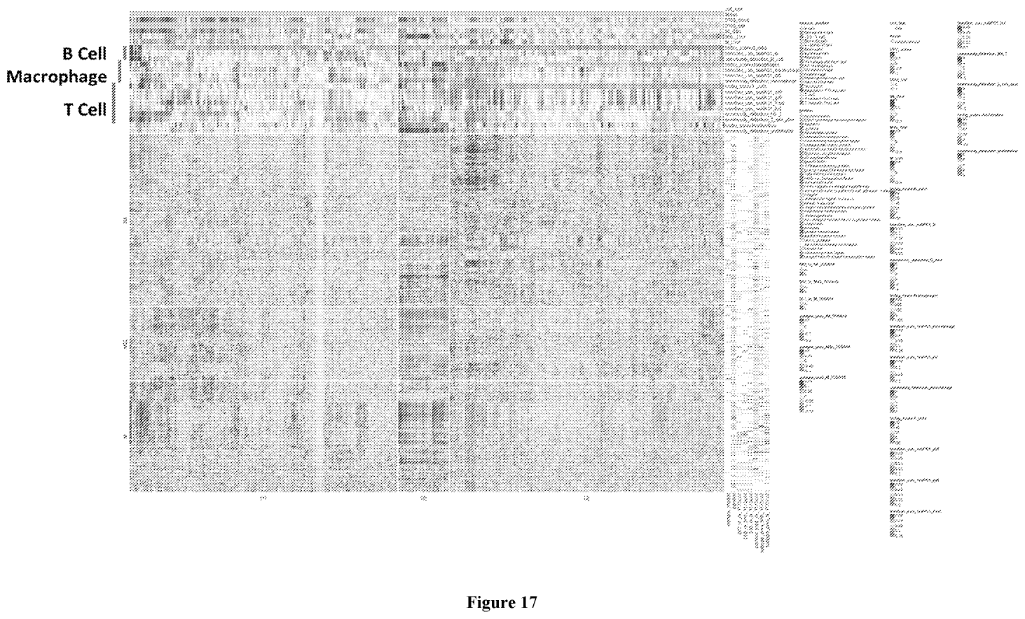

These three sets of distinct signatures, designated as ‘ImSig’, ‘ImmGen’ and ‘Community Detection’, show considerable conservation, as seen in a high correlation between most signatures sharing a B cell, T cell, or macrophage target (FIG. 15). As the community detection signatures identify sets of co-infiltrating immune cells, correlation of these signatures with multiple immune cell types may occur. The signatures were then used to classify clustered tumor expression data. Training data for lung adenocarcinoma and lung squamous cell tumors are shown (FIG. 16 and FIG. 17). In the lung adeno cluster the macrophage signature was strong for many of DetermaIO+/MSL cases and some also have T cell infiltrate signatures. B cell infiltrates appeared in other tumors, predominantly in the IM cluster. In the M cluster, there were a high number of un-activated T cell (as seen with the ImmGen ab and gd T cell signatures) infiltrates, while activated T cells (ImmGen activated T cell and ImSig and community detection T cell signatures) were mostly in IM. In the lung squamous cases, B cell infiltrates appeared rare outside of IM. However, distinct from lung adenocarcinoma cases, T cell infiltration was observed in IM and MSL, though high levels of un-activated T cells were still found in M.

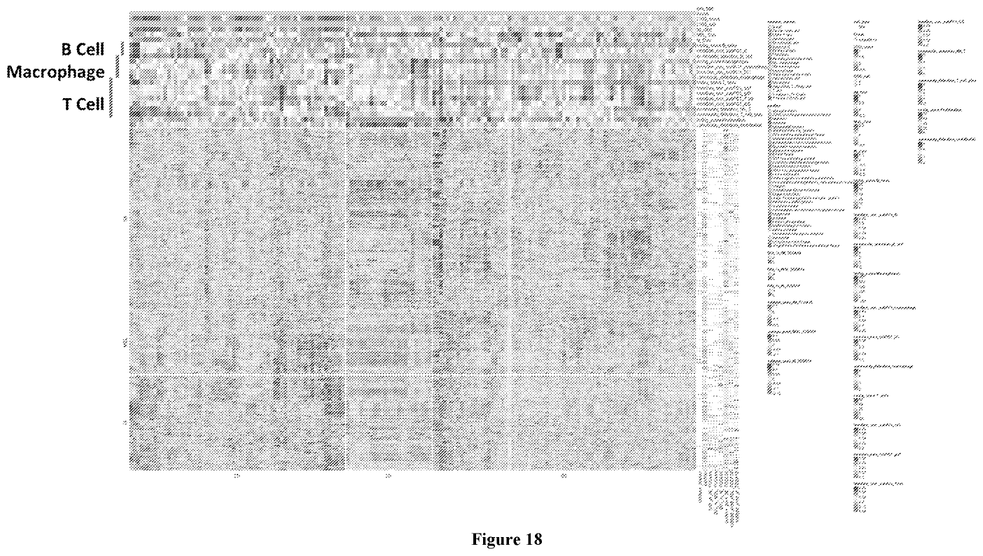

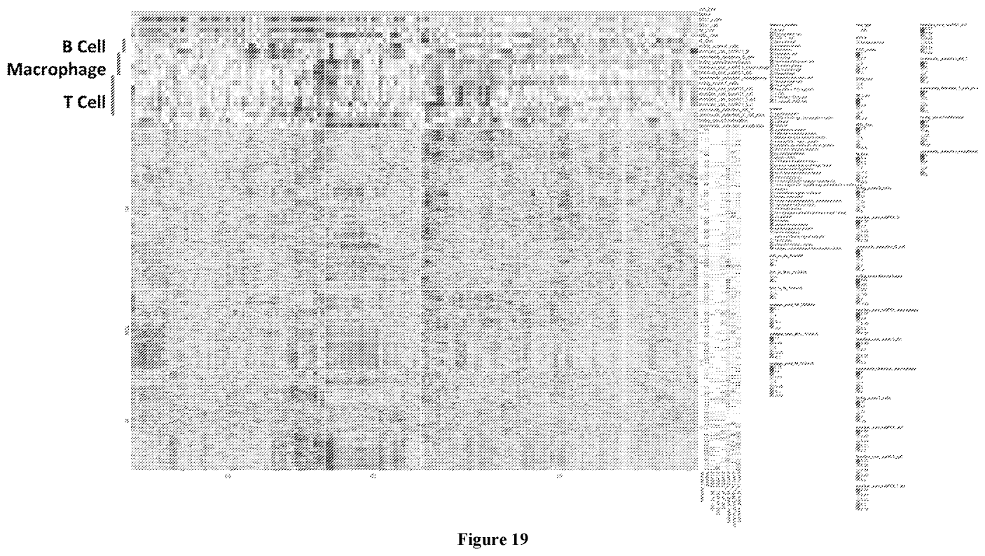

Similar distributions of immune signatures were observed in the test set of cases for these same tumors, both lung adenocarcinoma (FIG. 18) and lung squamous cell carcinoma (FIG. 19). For both tumors, macrophage infiltration was observed frequently in IM and MSL cases, and un-activated T cell infiltration was predominantly in M cases, while activated T cell infiltration was in the IM cluster (and in lung squamous, also in MSL), as seen in the training data.

Among other things, the present example demonstrates that immune infiltrate information may be determined through analysis of gene expression data and correlated with various tumor and gene expression subtypes (e.g., IM, M, MSL). In some embodiments, methods provided herein may be used to determine immune infiltrate levels for a particular tumor type without the need for a solid tumor biopsy. In some embodiments, immune infiltrate information may be used to inform or select one or more therapies for a tumor. In some embodiments, immune infiltrate information may be used in combination with tumor gene expression subtype data and/or DetermaIO scoring to inform or select one or more therapies for a tumor. In some embodiments, immune infiltrate information may be used in combination with tumor gene expression subtype data and/or DetermaIO scoring to identify patients whose cancer may not be adequately met by existing therapeutic regimens, or otherwise are strong candidates for novel drug discovery and development programs.

### Example 17: Scoring Based Upon Gene Methylation

The present Example, among other things, demonstrates that gene methylation status may be utilized in score prediction or other characterization of technologies as described herein (e.g., assessment of likely responsiveness to IO therap(ies), selection and/or modification of administered therap(ies) (including combination therap(ies)), monitoring of tumor development and/or evolution, etc).

Gene expression and gene methylation (Beta values—where beta 0 means unmethylated and beta of 1 means fully methylated) datasets for five cancers were downloaded from the genomic data commons (GDC) portal (https://portal.gdc.cancer.gov/) using the Genomic Data Commons package in R. Assessed cancers included Bladder Urothelial Carcinoma (BLCA), Breast invasive carcinoma (BRCA), Colon Adenocarcinoma (COAD), Lung Adenocarcinoma (LUAD), and Lung squamous cell carcinoma (LUSC). Subtype (IM, M, MSL) was calculated as previously described for each sample in the dataset. Cancer datasets (BLCA, BRCA, COAD, LUAD and LUSC) were then split, with 5% of each dataset reserved to create a small validation set (n=130) and 95% concatenated to create the training set (n=2026).

With the training set established, calculations were performed to generate the spearman rho correlation coefficient between each gene's beta values across all samples and subtype (IM, M, MSL), yielding three spearman rho values. Spearman rho values with a p value of <. 05 were removed from the set. Three lists of genes were generated containing 75 genes with the highest spearman rho in a corresponding subtype (e.g., 75 genes for each of IM, M, and MSL). A total of 214 unique genes were obtained from this assessment. Genes whose methylation status (beta value) were highly correlated with a particular subtype (IM, M, MSL) were selected as described in Example 15 and are outlined in Table 29 below.

TABLE 29
Genes with particularly elevated methylation levels that correlate with a subtype (IM, M, MSL).

| Gene | Subtype | Gene | Subtype | Gene | Subtype | Gene |
|---|---|---|---|---|---|---|
| TRAF1 | M | APOBEC3A | MSL | BATF | M | GRB7 |
| S100A9 | MSL | PCNAP1 | M | LGALS9B | MSL | CD101 |
| CD3E | M | ATIC | MSL | MIR770 | MSL | RARRES3 |
| HIST4H4 | MSL | A2ML1 | MSL | EPHA1 | MSL | GSR |
| WARS | MSL | BST2 | M | CNDP2 | MSL | TSTD1 |
| GRIK4 | MSL | VAMP1 | M | TM4SF1 | MSL | HN1 |
| MTMR11 | IM | NME6 | M | C17orf44 | IM | BCL2L2 |
| RHOB | IM | CSNK1E | IM | CORO1A | M | CRB3 |
| OAS2 | M | C17orf87 | M | MPL | IM | DCAF5 |
| RNF167 | M | VAMP8 | MSL | MIR92A1 | MSL | TUBA1C |
| RHBDL2 | IM | RAB13 | IM | MIR19A | MSL | PHKG1 |
| C10orf107 | IM | RAVER1 | IM | SEC16B | MSL | GRHL2 |
| HS1BP3 | IM | RILP | IM | BCL9L | IM | SP140L |
| HLA-E | M | TNFSF8 | M | SIT1 | M | LNX1 |
| IFNAR1 | M | TMC6 | M | ARNTL2 | MSL | CHMP4C |
| RTKN | IM | ELOF1 | M | BOK | IM | MGC3771 |
| ZNF641 | IM | CD3D | M | CYP4F3 | MSL | EVL |
| TRIM69 | M | PARP4 | MSL | KSR1 | MSL | LGALS8 |
| SPN | M | LOC257358 | M | PTPRCAP | M | ROD1 |
| AGAP2 | IM | LTBR | IM | FNBP1 | M | MIR142 |
| LOC100131496 | IM | CFLAR | M | RHOH | M | MMP14 |
| ESRP2 | IM | DENND2D | M | S100A8 | MSL | ASF1A |
| GGPS1 | M | C11orf34 | MSL | LOC150568 | MSL | CST7 |
| HTATIP2 | MSL | MIR18A | MSL | MIR1251 | MSL | ACAP1 |
| SLA2 | M | FASLG | M | GPD2 | IM | TNFSF13 |
| MAP1LC3B2 | MSL | LOC100130776 | IM | LTA | M | MIR17 |
| MON1A | IM | DIABLO | M | STK38L | MSL | CTNND1 |
| ABHD10 | IM | ADAM28 | MSL | AGTRAP | IM | ST7OT4 |
| KDELR3 | IM | C21orf15 | MSL | HMGA1 | MSL | LOC100233209 |
| SSH3 | IM | ENO1 | MSL | MIR22 | IM | 8-Sep |
| TNFRSF10A | MSL | KRTDAP | MSL | TAP1 | M | C14orf33 |
| MIR20A | MSL | EVI2A | M | PAPPA2 | MSL | FLJ13224 |
| MIR1252 | M | IRF1 | M | CXCR6 | M | PPP1R14B |
| GSDMC | MSL | LAX1 | M | KMO | M | SYS1 |
| Subtype | Gene | Subtype | Gene | Subtype | Gene | Subtype |
| IM | BTN3A3 | M | ACVR1 | IM | Clorf150 | MSL |
| M | INPP5J | IM | MOBKL2A | M | CHML | M |
| M | HIF1A | MSL | PRAME | MSL | C10orf91 | MSL |
| MSL | SLC25A15 | IM | SLC44A2 | IM | ZC3HAV1L | MSL |
| MSL | MPHOSPH9 | M | MIR155 | M | COMTD1 | MSL |
| MSL | UBE2L6 | M | P4HA2 | IM | GRIA1 | MSL |
| IM | UNC13C | MSL | WDR34 | IM | NFU1 | IM |
| IM | C5orf56 | M | VRK2 | MSL | HLA-B | M |
| IM | LOC285692 | MSL | MIR19B1 | MSL | TTC33 | MSL |
| MSL | LOC648691 | MSL | C15orf52 | IM | GAPDH | MSL |
| IM | PLCD4 | IM | OR2G3 | MSL | SNORD93 | MSL |
| MSL | FRK | IM | CRK | IM | PSMB8 | M |
| MSL | RAB19 | MSL | MIR609 | M | PTPN6 | M |
| MSL | TMEM159 | IM | IGFBP4 | IM | CLEC2D | M |
| MSL | C17orf91 | IM | TEC | MSL | SLC2A1 | MSL |
| IM | ZC3HAV1 | M | C2orf29 | MSL | FAM96B | IM |
| M | SNX11 | M | RARA | IM | TNFSF12- | IM |
| | | | | | TNFSF13 | |
| IM | DEGS1 | MSL | CDH1 | IM | EVI2B | M |
| MSL | RCAN3 | M | MPZL2 | IM | ACTR3C | MSL |
| M | ST7OT1 | IM | SLPI | MSL | | |
| IM | NLRC5 | M | PRSS8 | IM | | |
| MSL | TAGAP | M | LOC400931 | IM | | |
| MSL | GPR65 | M | LOC338799 | IM | | |
| IM | CD2 | M | KCTD11 | IM | | |
| IM | TBC1D10C | M | TMEM149 | M | | |
| MSL | GBP4 | MSL | B2M | M | | |
| IM | BTLA | M | SLC25A23 | IM | | |
| IM | TEF | IM | CD52 | M | | |
| M | CORO1B | M | TNKS1BP1 | IM | | |
| IM | MIR17HG | MSL | OCLN | IM | | |
| IM | AIP | M | SLC39A2 | IM | | |
| MSL | EIF2C4 | IM | LAMB2 | IM | | |
| IM | SUOX | IM | RAP1A | M | | |
| M | MFSD6L | IM | DAPP1 | MSL | | |

Among other things, the present Example demonstrates that available gene methylation data may be utilized, and, in some embodiments, combined with the 101 gene signature, to produce a model to classify genes with abnormally high methylation levels (e.g., as compared to a reference) into a particular signature type (e.g., IM, M, MSL). In some embodiments, this model may be used to assess a sample of interest (e.g., tumor sample from a subject, blood and/or plasma sample from a subject) for gene methylation status (e.g., relative to a reference) in order to assess IM, M, and MSL signature levels. In some embodiments, assessment of gene signature levels could be leveraged to create a gene methylation-specific scoring system to inform selection and/or modification of therapy (e.g., ICI therapy, chemotherapy, etc.).

For example, in some embodiments, a tumor sample with one or more methylated genes correlating to a particular subtype (e.g., M, MSL) could be treated with one or more therapies to reduce or inhibit methylation (e.g., methyltransferase inhibitor, demethylase) and shift tumor status to a different subtype (e.g., IM). In some embodiments, one or more therapies to reduce or inhibit methylation (e.g., methyltransferase inhibitor, demethylase) may be combined with one or more additional therapies (e.g., ICI therapy, chemotherapy, etc.).

### Example 18: Scoring Based Upon Gene Methylation

The present Example, among other things, demonstrates that classifications provided herein can be correlated with promoter and/or gene methylation and expression level and may be utilized for and/or included in assessments of subjects and/or their immune statuses and/or responsiveness to therapy.

Potential promoter regions were identified using R (version 4.2.1) and the package GenomicFeatures and the TxDb.Hsapiens.UCSC.hg19.knownGene annotation package, applied to breast cancer data was from the TCGA. Potential promoter regions 100 kb upstream from the start of gene were selected for further analysis. Methylation probes for the promoter regions of the genome were then selected.

Each individual tumor sample was mapped to one or more of the IM, M, and MSL subtypes as described herein. Samples were then grouped to perform three different sets of comparisons: Group 1) Samples with an IM subtype as compared to those with an M or MSL subtype; Group 2) Samples with an IM subtype as compared to those with an MSL subtype; and Group 3) Samples with an MSL subtype as compared to those with an M subtype.

For each group, a logistic regression model was fit for each gene to identify candidate genes that could be drivers of transition from one immune state to another (e.g., IM to M/MSL or vice versa (Group 1 above), IM to MSL or vice versa (Group 2 above), M to MSL or vice versa (Group 3 above)). Each model used the promoter with largest standard deviation in methylation beta values as the feature to predict subtype. The p-value for the feature was stored for each of these models. A Benjamin-Hochberg correction was applied to select the “best” models. A linear regression model was then generated for each probe to predict gene expression based upon methylation value. These analyses were intended to confirm that methylation status of a promoter corresponds to expression of a potential target gene. Mean absolute percentage error (MAPE) was calculated and stored for each model. Genes whose MAPE was in the bottom quantile of each Group were kept for further analysis, as shown in Table 30, Table 31, and Table 32 below.

TABLE 30
Gene list produced after MAPE filtering of Group 1 genes.
Gene

| | | | | | | |
|---|---|---|---|---|---|---|
| ARHGEF10 | SLC2A7 | SPATA5L1 | ZNF230 | TMEM30A | PIRT | HIVEP3 |
| TPRKB | MCM2 | ANP32E | CXCR4 | HIP1R | ITGA1 | RAB17 |
| SGSM2 | KRT27 | CDC42EP4 | ANXA6 | CRYZ | EVX2 | ACP2 |
| SKAP2 | VGLL4 | TGM7 | SKA2 | CASP2 | MAD1L1 | EID1 |
| ZMIZ2 | PYROXD1 | CDK2AP1 | GALNT11 | SPRR2F | CKAP5 | LAMB2 |
| DZIP3 | GTF2H5 | B4GALT2 | POLL | KLHDC8B | B3GNT6 | LPP |
| DNAJB12 | TGFBI | RUNX1 | ZC3H11A | TNFAIP2 | SELENBP1 | PLXDC2 |
| PELO | KHNYN | Clorf198 | DDAH1 | CD81 | PPP2R2D | RALBP1 |
| GOLPH3L | NCK1 | SEC14L1 | RBMS2 | TRAF3IP2 | CSNK1A1L | LMO7 |
| SLC25A46 | RUSC1 | SLC37A1 | MON1A | LIMA1 | ITPRIPL2 | SLC29A1 |
| ALDH7A1 | LAMA4 | FKBPL | SIGIRR | SPARC | BBS7 | RANBP1 |
| DIXDC1 | TPM3 | RAB11FIP3 | LPAR2 | EPB41L1 | SLC33A1 | MAP3K13 |
| ZNF562 | CCDC86 | AMPD2 | TRIOBP | ZNF8 | CYB5R4 | MKS1 |
| HNRNPF | TRIB2 | SULF2 | NSUN5 | MRPS27 | NEU1 | SYPL1 |
| ILVBL | ZNF207 | RASA3 | NCOR2 | NDUFA6 | EPHB4 | CDK10 |
| FLII | TMEM63A | IFITM3 | SH3BP2 | USP6NL | TRIM4 | ILF2 |
| KDR | HSPA1B | UNC45A | ZNF440 | ZNF581 | SNX1 | TNFSF12 |
| C1RL | CYTH3 | ANO6 | LIMS1 | CFL2 | SOCS4 | PPEF2 |
| MEF2D | TUT1 | FKBP1A | MLH3 | S100A10 | ADAM18 | ACVRL1 |
| MTX3 | SPATA13 | IL17RC | MCM3AP | STK36 | DNAJC14 | GLUL |
| COMT | PRDM7 | C11orf58 | OR2H2 | PTP4A1 | TMEM129 | LARP1 |
| LAMB1 | ZMYND8 | SHROOM3 | GGCT | PKN1 | NDUFAF3 | ASTE1 |
| STT3A | FLAD1 | FBXO6 | TM9SF4 | CDK5RAP1 | INPP5A | AGPAT1 |
| YIPF3 | BUD31 | MGAT1 | MBIP | NUFIP1 | WIPF1 | GNAI2 |
| FAM118A | ARCN1 | GUSB | CAST | CCNL2 | PLRG1 | ZNF621 |
| PTER | ZDHHC13 | SLC36A2 | PRRG2 | C10orf88 | ACTL6A | TCF12 |
| GSDMD | TRAK1 | MRPS21 | GPATCH8 | HLCS | PDIA4 | DUS1L |
| CARD8 | PNPLA2 | COX11 | AK3 | LDLR | NR2F2 | BID |
| LGALS3BP | GHITM | GPR152 | JMJD1C | PSD4 | NCOA4 | AGPAT3 |
| ZBED5 | GC | PGAP2 | RARA | DAAM1 | UBXN6 | OR8A1 |
| PXN | LMNA | WDR75 | RUFY3 | MAX | TMEM140 | BCL9L |
| TNPO1 | PALMD | NDUFS3 | FAR1 | PSMB7 | GANC | APLP2 |
| IRF6 | ATP6V0A1 | LBH | GMFB | NUCB1 | ORAI3 | APBB2 |
| ARAP1 | KBTBD4 | C12orf40 | RAPGEF1 | CD44 | MFN2 | MLH1 |
| SHMT2 | FBXO40 | BCR | CRTC2 | TBC1D22B | ZMYND11 | CABLES1 |
| YIPF5 | AP2A2 | RHOD | ZNF600 | SPINK7 | ROBO4 | PSMC5 |
| FSCN1 | EPM2AIP1 | GPS2 | RAB4A | MUL1 | ELOVL5 | ZNF787 |
| FAM107B | TMC6 | KRT8 | PACSIN2 | MGST2 | PDCL3 | SLC38A10 |
| NBR1 | ITGB2 | PPM1M | HSPA4 | PPOX | GPN1 | IRX5 |
| LGALS1 | GOLIM4 | SLC27A3 | CLSTN1 | COL1A1 | PEX5 | GORASP2 |
| MARCKS | TFAP2A | ETS2 | UBE2U | CRELD1 | OTX2 | SDC2 |
| NUDT16 | CPT2 | MORC1 | BBX | PTPN12 | FKBP10 | MED18 |
| ZNF561 | VGLL2 | GRHL2 | STAB1 | TPM1 | BCL2L13 | AHSP |
| C11orf24 | MPZL3 | MAF | SHC1 | CD74 | RNASEL | ISY1 |
| CALHM2 | MCRS1 | ZNF358 | FBXL19 | LDHAL6B | ZNF639 | STK38L |
| WWTR1 | CD5L | EPS8L2 | RPS6KA2 | ANKH | NUMA1 | ARHGEF7 |
| SCOC | PPP1R9B | FAM111A | NUBP1 | FAM193A | GH1 | PARVA |
| NSUN3 | C15orf39 | PTPRK | PGK2 | TNIP2 | OCIAD2 | KNCN |
| WIPI2 | FKBP14 | BARHL2 | HIPK1 | DDX46 | SYNPO | RPL32 |

| | | | | |
|---|---|---|---|---|
| KIF22 | CIZ1 | COL15A1 | HSP90AB1 | RCOR3 |
| OCIAD1 | SPEM1 | NDUFC2 | UBLCP1 | TXNRD1 |
| C1QC | HSD3B7 | TTC14 | CDYL | TRIM22 |
| TNFAIP8L1 | NUPR1 | PSMG1 | DDX1 | XPO1 |
| TSPAN4 | NBL1 | UBB | CALD1 | SCLT1 |
| CDC25B | ATCAY | RRP7A | PPP1R15B | ARHGAP27 |
| FUT11 | PARP14 | MTUS1 | TCF21 | TMEM18 |
| GPR137 | ABHD12 | TMX4 | CEP97 | CCNG1 |
| LPIN2 | AP1M2 | CFLAR | MTIF3 | BCAP29 |
| GNB2 | S100A16 | TNS1 | ETS1 | HSD17B4 |
| DHCR24 | MARVELD2 | BAIAP2 | LNX1 | UBXN11 |
| ESAM | SRRM1 | NSMCE1 | SCFD1 | CASKIN2 |
| LGALS8 | LMBRD1 | EHD2 | ZCCHC14 | RPP30 |
| DCUNID3 | USP47 | SH3YL1 | RPS6KA1 | TRAPPC9 |
| FOXRED1 | PIGC | POLD2 | KIAA1217 | RIN2 |
| MYOF | NXPH2 | WHAMM | DENND2D | THBS1 |
| MAP3K11 | FRG1 | LYPLAL1 | SPRED2 | LYN |
| SRP19 | SLMAP | ZNF320 | GAB1 | SNX25 |
| TCEA2 | RNF34 | VIM | ZSCAN29 | TAF7 |
| CD93 | RAB11B | PURA | ABHD14B | TPRA1 |
| DDR1 | ZNF558 | PHOX2A | TLE3 | PTPRB |
| AGK | BTBD1 | CRX | SEC13 | PROM2 |
| DPH2 | HOOK1 | TMBIM1 | TMC4 | DCTN3 |
| GPATCH3 | IFI16 | HTR6 | FAM110A | GSTO1 |
| PSMD6 | MKNK2 | PNLIP | EIF5A | ECE1 |
| PELI1 | RAI1 | DSPP | RNF25 | TBRG4 |
| JARID2 | KDM2B | YTHDF2 | ANKRD11 | POGZ |
| YWHAQ | USP33 | PARP6 | TRMT12 | DTX3 |
| CLUAP1 | WFDC10A | SCPEP1 | LCN15 | TMBIM4 |
| DNMT3A | IFT122 | NBEAL2 | HERC2 | TIMP2 |
| NDUFS2 | RPL23A | CST3 | PHACTR4 | RBBP9 |
| VARS2 | INPP5B | RPS7 | DNAJB1 | TTC38 |
| PTPRF | ALDOA | BCL9 | TGFBR2 | AP4S1 |
| B3GNT9 | ZNF385A | RBM47 | ZNF44 | PKP3 |
| MGAT4B | OR10G2 | DIP2C | FANCF | GALR3 |
| TXN2 | PTPN6 | ARHGEF10L | ALG10B | FFAR3 |
| TTC27 | BTNL2 | NAGK | RPTOR | CRY2 |
| PLEKHA6 | SMAD3 | EPS8 | NET1 | MFSD1 |
| TOMM40L | SMTN | TPM4 | CCDC50 | MAPK6 |
| POLG2 | USP54 | NXNL1 | KRT85 | GSS |
| ATN1 | UBA5 | TNKS1BP1 | CDK2 | |
| KDM3B | SERPINB1 | SLC12A4 | KCTD12 | |
| SCD | UBE2E1 | PRKAG2 | BTBD17 | |
| SFTPA1 | NUDT19 | SLC44A2 | SLC11A2 | |
| TKTL2 | PTK2 | MRPL9 | PAPOLB | |
| COASY | NME3 | PAAF1 | TNPO2 | |
| EIF3E | WDR6 | AMBRA1 | TMEM69 | |
| RHOT2 | POLR1C | RFFL | PDE8A | |
| STK16 | UACA | PRCP | ABHD8 | |

TABLE 31
Gene list produced after MAPE filtering of Group 2 genes.
Gene

| | | | | | |
|---|---|---|---|---|---|
| TNPO1 | LGALS3BP | NCOA4 | DDX46 | DNAJB12 | SULF2 |
| RALGAPB | SLMAP | DIXDC1 | CABLES1 | DZIP3 | TNIP2 |
| CRELD1 | SHMT2 | ZNF207 | TXNRD1 | CCDC86 | NBEAL2 |
| PSMB8 | GSN | SLC27A3 | PARP14 | PPEF2 | CD5L |
| RHOD | PNPLA2 | ARCN1 | MGST2 | SEC13 | ELOVL5 |
| CTRL | SMAD3 | FBXO38 | ZNF558 | RPL23A | DCUN1D4 |
| ZNF600 | CARD8 | PSMD5 | CRX | RFFL | SPRR2F |
| ALDH4A1 | LDHAL6B | PGAP2 | TAF7 | NUCB1 | FBXL19 |
| DCUN1D3 | LPIN2 | ZNF358 | ANKRD11 | MARVELD2 | PIRT |
| PPP2R2D | DDR1 | PSMC5 | VGLL2 | TMEM140 | TCN2 |
| ARAP1 | RNASEL | ANXA6 | APC | LPAR2 | TRAF3IP2 |
| KRT8 | THBS1 | CDK2 | CALD1 | EID1 | HERC2 |
| C1QC | DDAH1 | HIVEP3 | HLCS | GPATCH8 | DCTN1 |
| DNAJC14 | ZNF621 | PARVA | BCL2L13 | PDIA4 | POLG2 |
| TNFAIP8L1 | ABHD14B | FBXO40 | SLC24A5 | TNFSF12 | AP4S1 |
| BCR | AXL | TMX4 | FKBP10 | MAD1L1 | SKA2 |
| TLE3 | BRD9 | C15orf39 | TMEM30A | FFAR3 | HIP1R |
| CDK10 | ABRA | FAM107B | PTGR1 | TBRG4 | CALHM2 |
| ANP32E | ITGB2 | DSPP | KBTBD4 | SPARC | PRDM7 |
| GSTO1 | PPFIBP2 | FOXE3 | MGAT1 | ATCAY | GOLIM4 |
| BUD31 | MPZL3 | TUT1 | CCNL2 | ZNF385A | UBE2E1 |
| TKTL2 | SLC29A1 | ORAI3 | HTR6 | NDUFAF3 | ATN1 |
| RAB11FIP3 | CD44 | PPM1M | ITPRIPL2 | PROM2 | PPP1R9B |
| EPHB4 | NXPH2 | LAMB2 | NAGK | SERPINB1 | CYTH3 |
| YTHDF2 | NDUFS2 | PTPN6 | ISY1 | TOMM40L | OR2H2 |
| GRAMD4 | MEF2D | FAM110A | PAPOLB | ZNF787 | KDM2B |
| ALDOA | GRHL2 | USP47 | CEP97 | TIMP2 | RBM47 |
| ALX3 | TNFRSF1B | GUSB | ATM | LBH | SGSM2 |
| XPO1 | RASSF1 | NET1 | LYPLAL1 | MLH3 | TNPO2 |
| COASY | TCF21 | RBM5 | CRYZ | ALCAM | FOXRED1 |
| C1RL | MTIF3 | CD81 | SLC36A2 | POLL | IFITM3 |
| ACTL6A | PHACTR4 | AHSP | EPHX1 | HSP90AB1 | FLII |
| STAB1 | TRIB2 | CFL2 | GALR3 | TSC22D1 | RALBP1 |
| GPS2 | LMNA | ACP2 | NEU1 | SLC44A2 | USP6NL |
| HSPA1B | LAMA4 | SLC38A10 | ATP4B | LARP1 | ILF2 |
| BARHL2 | TRIM22 | TPM1 | SLC2A7 | C1S | TBC1D22B |
| SH3BP2 | PGK2 | PIGC | KCTD12 | NID1 | CDYL |
| PURA | SCPEP1 | TPRA1 | CPT2 | BTBD1 | PSMG1 |
| PELO | MRPS27 | TSPAN4 | WHAMM | TFAP2A | AK3 |
| SFTPA1 | FUT11 | ABHD12 | ZCCHC14 | S100A13 | RPP30 |
| L3MBTL2 | SCLT1 | CRTC2 | RRP7A | LDLR | IFI16 |
| OTX2 | ATP6V0A1 | GSS | LCN15 | MMRN2 | PLEKHA6 |
| PNLIP | HSPA4 | UBLCP1 | UBXN11 | RASA3 | RUNX1 |
| OR8A1 | MON1A | YIPF5 | ANO6 | CKAP5 | MGAT4B |
| JARID2 | PTPRK | PTK2 | MTX3 | RFPL1 | DNMT3A |
| MORC1 | AGPAT1 | ZNF562 | AGK | Clorf210 | TARBP2 |
| ACACA | DNAJB1 | RNF25 | SPINK7 | LHX5 | BBX |
| ZNF721 | FLAD1 | CAST | KHNYN | ETS1 | TTC38 |
| GTF2H5 | TMBIM1 | TNKS1BP1 | EPS8L2 | INPP5B | UBXN6 |
| ARRDC2 | PXN | WSB1 | PDE7A | S100A13 | HECW2 |
| BCAS3 | BRD4 | ZNF350 | SLC38A8 | HEXA | CABLES1 |
| ZNF560 | ZNF254 | MORC2 | STK38L | NME2 | YTHDF2 |
| DNAJB12 | PPFIBP2 | AMIGO3 | COX7C | SFXN1 | FLII |
| OR10G2 | SIAH1 | TJAP1 | RNF34 | PARVB | DNAJC5 |
| STK19 | KIR3DX1 | RGS16 | TTC1 | MFSD1 | CD93 |
| ECE1 | MGST2 | ZNF621 | AHR | PDXK | CYB561 |
| BRD9 | NEURL4 | S100A6 | NUCB1 | ATP6V0A1 | PSMG1 |
| RALGDS | PTGS1 | HSP90AB1 | GTSF1L | NIPA1 | FURIN |
| TSTD1 | CCNDBP1 | ANO6 | OCIAD2 | ITPR3 | LMBRD1 |
| BMF | SELENBP1 | MMRN2 | PELI1 | SLC25A2 | EIF5A |
| TMCO1 | PSG3 | GRAMD4 | TJP2 | SIRT6 | ZNF44 |
| SHC1 | ICA1 | GOLIM4 | RAB11FIP3 | KPRP | RAB4A |
| UBXN6 | RAB11B | C10orf105 | C12orf40 | NRP2 | MYL12A |
| CCDC50 | POGZ | ABCG1 | ZNF440 | RASA3 | FKBP10 |
| Clorf198 | TGFBR2 | CKAP5 | RNASEL | MRPL3 | TRERF1 |
| ATM | AIPL1 | ILVBL | TAF7 | MNAT1 | WDR82 |
| ZFP41 | GNAI2 | AP2A2 | ZNF787 | CCDC149 | SYPL1 |
| GNB2 | PNLIP | TBRG4 | PHACTR4 | PRKACA | ST7 |
| FSCN1 | CTRL | CDYL | RNF220 | | |

| | | | | | |
|---|---|---|---|---|---|
| CST3 | ZNF8 | SOCS4 | RAI1 | FANCF | FKBP14 |
| MAX | NUDT16 | EPB41L1 | PKN1 | BCAP29 | HIPK1 |
| BAIAP2 | NSMCE1 | KDM3B | OCIAD1 | SLC33A1 | LIMS1 |
| PMP22 | MYOF | POLD2 | ETS2 | GAB1 | FMOD |
| COMT | ZNF26 | ILVBL | TRAK1 | MKNK2 | KIAA1217 |
| HOOK1 | RAB4A | DTX3 | ASB5 | TMBIM4 | AP1M2 |
| RAPGEF1 | SPATA13 | NUDT19 | TPRKB | IPO5 | ZMYND11 |
| COX8A | LGALS1 | MUL1 | IL17RC | KRT27 | BCL9 |
| ZNF518A | PRRG2 | GORASP2 | DUS1L | FSCN1 | RIN2 |
| SHROOM3 | NDUFA6 | LIMA1 | B4GALT2 | SCFD1 | PPP1R15B |
| FKBPL | TTC14 | SLC25A46 | TMEM63A | EHD2 | ZMIZ2 |
| TCEA2 | PXN | TGFBI | CRY2 | TGM7 | PSMB7 |
| PALMD | ACVRL1 | CD74 | IRF6 | ZC3H11A | GPATCH4 |
| TSKU | NUPR1 | RNF34 | CD93 | APLP2 | RARA |
| SELENBP1 | SYPL1 | CCNDBP1 | RUSC1 | TGFBR2 | MRPL3 |
| ALDH7A1 | SEC14L1 | TRIM4 | STK36 | MAP3K11 | SPEM1 |
| BTBD17 | KIF22 | NCK1 | EIF3E | NFATC4 | GNAI2 |
| SPG21 | CXCR4 | STK38L | UBE2U | ZNF44 | KLHDC8B |
| TRIOBP | EVX2 | TMC6 | NXNL1 | POLR1C | TMC4 |
| RALGDS | UACA | BID | PTPRF | RAB17 | ZNF561 |
| PELI1 | ZSCAN29 | PARP6 | ARHGEF10L | NBR1 | WDR6 |
| NDUFS3 | PSD4 | BMF | RPS6KA1 | STT3A | ZBED5 |
| SPATA5L1 | MCRS1 | BBS7 | INPP5A | COX11 | MCM2 |
| AP2A2 | DHCR24 | BCL9L | ALG10B | C11orf24 | RCOR3 |
| LYN | WWTR1 | PDE8A | CDK2AP1 | FXYD5 | SH3YL1 |
| TRIO | C11orf58 | EIF5A | RBMS2 | OR10G2 | TNAIP2 |
| LMBRD1 | PSMD6 | ITGA1 | SLC37A1 | DCTN3 | PDCL3 |
| SNX1 | TMEM129 | SPRED2 | JMJD1C | ARHGEF7 | PRKAR2A |
| LOXL1 | USP54 | GMFB | SIGIRR | COL15A1 | PTPRB |
| DENND2D | RPS7 | WIPI2 | VIM | PPOX | TMEM18 |
| MTUS1 | VARS2 | PTER | ZNF581 | GH1 | SLC11A2 |
| SRP19 | UBB | SLC12A4 | CIZ1 | WFDC10A | FAM118A |
| KNCN | GC | COL1A1 | NUBP1 | MKS1 | AMPD2 |
| TXN2 | PEX5 | GANC | LMO7 | IFT122 | GSDMD |
| PRCP | NUMA1 | SYNPO | APBB2 | PSMG2 | |
| ZMYND8 | CCNG1 | CDC42EP4 | GOLPH3L | ZNF639 | |
| NR2F2 | TRMT12 | PKP3 | RPS6KA2 | DDX1 | |
| TPM4 | LGALS8 | ESAM | PRKAG2 | AMBRA1 | |
| MYL9 | GPR142 | HPCAL1 | PTPN12 | ANKH | |
| GALNT11 | TPM3 | NDUFC2 | MAP3K13 | UBA5 | |
| ZDHHC13 | S100A10 | CASKIN2 | SNX25 | TTC27 | |
| RANBP1 | USP33 | MED18 | YIPF3 | DIP2C | |
| CSNK1A1L | YWHAQ | KNG1 | MRPL9 | AGPAT3 | |
| VGLL4 | CFLAR | RUFY3 | GHITM | PAAF1 | |
| GGCT | FAM193A | BANF1 | UNC45A | PLXDC2 | |
| CDK5RAP1 | RPTOR | LUZP1 | NSUN3 | NCOR2 | |
| FAM111A | SHC1 | B3GNT6 | ST7 | ZNF529 | |
| COL6A3 | ZNF320 | KRT85 | LNX1 | CLUAP1 | |
| KDR | IRX5 | Clorf198 | ADAM18 | CDC25B | |
| DDX1 | TMEM30A | | | | |
| HPCAL1 | GRHL2 | | | | |
| GPRC5C | SSH1 | | | | |
| GORASP2 | HNF1B | | | | |
| CD81 | ALDH7A1 | | | | |
| ZNF320 | IL10RB | | | | |
| RPS7 | TCF12 | | | | |
| DENND2D | GSN | | | | |
| RBM19 | FBXO6 | | | | |
| ZNF544 | UBE2E1 | | | | |
| GALR3 | KDM3B | | | | |
| FAM174B | TXNIP | | | | |
| FXYD5 | CCDC88A | | | | |
| RPL10L | THBS3 | | | | |
| MEF2D | ALDOA | | | | |
| IFT122 | CCDC86 | | | | |
| ZMIZ2 | ZNF430 | | | | |
| FAM118A | SRC | | | | |
| GBX1 | SETMAR | | | | |

The UniProt web API was then searched for genes from the analyses above (Table 30, Table 31, Table 32) with an associated keyword of “disease variant,” with results shown in Table 33, Table 34, and Table 35 below. Additionally, the Targetome database was utilized to determine which identified genes are also targets for one or more cancer therapies, as shown in Table 36, Table 37, and Table 38 below. Genes that contained the keyword of interest or were in the Targetome database were identified as genes of high interest for potential modulators of the immune state transition.

TABLE 33
Gene list produced after keyword filtering of Group 1 genes.
Gene

| | | | | | | | |
|---|---|---|---|---|---|---|---|
| ARHGEF10 | AMPD2 | CSNK1A1L | FOXRED1 | CRX | TNPO2 | MLH1 | KRT8 |
| TPRKB | GUSB | BBS7 | MAP3K11 | HTR6 | MAPK6 | IRX5 | GRHL2 |
| SLC25A46 | SLC36A2 | SLC33A1 | DDR1 | PNLIP | GSS | STK38L | MAF |
| ALDH7A1 | CXCR4 | NEU1 | AGK | DSPP | PTK2 | DNMT3A | FAM111A |
| KDR | TRIOBP | EPHB4 | DPH2 | PPP1R15B | POLR1C | NDUFS2 | RARA |
| COMT | SH3BP2 | NDUFAF3 | GPATCH3 | RPS6KA1 | CST3 | VARS2 | FAR1 |
| STT3A | MLH3 | ACTL6A | CIZ1 | GAB1 | PRKAG2 | IRF6 | RPS6KA2 |
| CARD8 | TRAF3IP2 | NR2F2 | HSD3B7 | EIF5A | HERC2 | SHMT2 | HIPK1 |
| MCM2 | SPARC | LAMB2 | ATCAY | ANKRD11 | POLG2 | YIPF5 | MAX |
| PYROXD1 | EPB41L1 | MAP3K13 | ABHD12 | TXNRD1 | ATN1 | NSUN3 | PSMB7 |
| GTF2H5 | NDUFA6 | MKS1 | PIGC | HSD17B4 | KDM3B | WIPI2 | PPOX |
| TGFBI | CFL2 | ACVRL1 | RAB11B | LYN | SFTPA1 | GC | COL1A1 |
| LAMA4 | STK36 | GLUL | MKNK2 | ECE1 | COASY | LMNA | CRELD1 |
| TPM3 | PKN1 | TCF12 | NDUFC2 | POGZ | STK16 | ITGB2 | PTPN12 |
| TMEM63A | HLCS | KIF22 | RRP7A | TGFBR2 | IFT122 | TFAP2A | TPM1 |
| FLAD1 | LDLR | C1QC | MTUS1 | NET1 | ALDOA | CPT2 | ANKH |
| TRAK1 | MAD1L1 | LPIN2 | VIM | KRT85 | SMAD3 | PGAP2 | MFN2 |
| PNPLA2 | B3GNT6 | GNB2 | PURA | CDK2 | UBA5 | NDUFS3 | ROBO4 |
| RUNX1 | SELENBP1 | DHCR24 | PHOX2A | SLC11A2 | GH1 | BCR | ELOVL5 |
| PEX5 | OTX2 | FKBP10 | | | | | |

TABLE 34
Gene list produced after keyword filtering of Group 2 genes.
Gene

| | | | | | | | |
|---|---|---|---|---|---|---|---|
| CRELD1 | PNPLA2 | NET1 | RRP7A | CST3 | CXCR4 | B3GNT6 | TGFBR2 |
| PSMB8 | SMAD3 | CFL2 | AGK | MAX | DHCR24 | KRT85 | MAP3K11 |
| ALDH4A1 | CARD8 | TPM1 | MAD1L1 | PMP22 | VARS2 | PKN1 | POLR1C |
| KRT8 | LPIN2 | PIGC | SPARC | COMT | GC | TRAK1 | STT3A |
| C1QC | DDR1 | ABHD12 | ATCAY | SELENBP1 | PEX5 | TPRKB | PPOX |
| BCR | AXL | GSS | NDUFAF3 | ALDH7A1 | TPM3 | TMEM63A | GH1 |
| EPHB4 | ITGB2 | YIPF5 | MLH3 | TRIOBP | IRX5 | IRF6 | MKS1 |
| ALDOA | NDUFS2 | PTK2 | C1S | NDUFS3 | EPB41L1 | STK36 | IFT122 |
| ALX3 | GRHL2 | TXNRD1 | TFAP2A | LYN | KDM3B | RPS6KA1 | ANKH |
| COASY | LMNA | CRX | LDLR | TRIO | SLC25A46 | VIM | UBA5 |
| ACTL6A | LAMA4 | ANKRD11 | ELOVL5 | MTUS1 | TGFBI | CIZ1 | HIPK1 |
| SH3BP2 | FLAD1 | APC | TRAF3IP2 | NR2F2 | STK38L | RPS6KA2 | PPP1R15B |
| PURA | FBXO38 | HLCS | HERC2 | CSNK1A1L | BBS7 | PRKAG2 | PSMB7 |
| SFTPA1 | PGAP2 | FKBP10 | POLG2 | FAM111A | EIF5A | PTPN12 | RARA |
| OTX2 | CDK2 | HTR6 | ATN1 | COL6A3 | WIPI2 | MAP3K13 | MRPL3 |
| PNLIP | DSPP | ATM | TNPO2 | KDR | COL1A1 | NSUN3 | MCM2 |
| GTF2H5 | FOXE3 | SLC36A2 | FOXRED1 | NDUFA6 | NDUFC2 | SLC33A1 | SLC11A2 |
| SHMT2 | LAMB2 | NEU1 | RUNX1 | ACVRL1 | KNG1 | GAB1 | AMPD2 |
| GSN | GUSB | CPT2 | DNMT3A | KIF22 | BANF1 | MKNK2 | |

TABLE 35
Gene list produced after keyword filtering of Group 3 genes.
Gene

| | | | | |
|---|---|---|---|---|
| BCAS3 | SELENBP1 | SIRT6 | MORC2 | SLC38A8 |
| ECE1 | RAB11B | MRPL3 | HECW2 | STK38L |
| TMCO1 | POGZ | PRKACA | DNAJC5 | AHR |
| ATM | TGFBR2 | GRHL2 | CYB561 | |
| GNB2 | AIPL1 | HNF1B | EIF5A | |
| TJP2 | PNLIP | ALDH7A1 | FKBP10 | |
| RPL10L | HEXA | TCF12 | ALDOA | |
| IFT122 | PDXK | GSN | SRC | |
| SIAH1 | NIPA1 | KDM3B | RNF220 | |

TABLE 36
Gene list filtering Group 1 genes for those identified as targets for
one or more cancer therapies.
Gene

| | | | | | | |
|---|---|---|---|---|---|---|
| KDR | PKN1 | ACVRL1 | MKNK2 | LYN | PTK2 | RARA |
| COMT | CSNK1A1L | C1QC | HTR6 | TGFBR2 | STK16 | RPS6KA2 |
| CXCR4 | EPHB4 | MAP3K11 | RPS6KA1 | CDK2 | STK38L | HIPK1 |
| STK36 | MAP3K13 | DDR1 | TXNRD1 | MAPK6 | BCR | PSMB7 |

TABLE 37
Gene list filtering Group 2 genes for those identified as targets for
one or more cancer therapies.
Gene

| | | | | | | |
|---|---|---|---|---|---|---|
| PSMB8 | DDR1 | TXNRD1 | LYN | CXCR4 | RPS6KA1 | TGFBR2 |
| C1QC | AXL | HTR6 | CSNK1A1L | STK38L | RPS6KA2 | MAP3K11 |
| BCR | CDK2 | C1S | KDR | PKN1 | MAP3K13 | HIPK1 |
| EPHB4 | PTK2 | COMT | ACVRL1 | STK36 | MKNK2 | PSMB7 |
| RARA | | | | | | |

TABLE 38
Gene list filtering Group 3 genes for those
identified as targets for one or more cancer therapies.
Gene

| | | |
|---|---|---|
| TGFBR2 | SRC | AHR |
| PRKACA | STK38L | |

In some embodiments, the present example demonstrates that methylation status of certain genes and/or gene promoters may increase IM, M, and/or MSL character of a tumor. In some embodiments, changes in methylation status (e.g., increased or decreased methylation) of certain genes and/or gene promoters may drive a change in subtype character of a tumor. In some embodiments, changes in methylation status (e.g., increased or decreased methylation) of certain genes and/or gene promoters may drive a change from M and/or MSL subtype to IM subtype. In some embodiments, a treatment may be selected to produce changes in methylation status for certain genes and/or gene promoters, for example in order to increase or decrease methylation of genes associated with IM (e.g., potentially increasing IM subtype character of a tumor and/or DetermaIO scoring).

### Example 19: Assessment of Data from miRNA Targeting Exemplary Gene Sets

The present Example, among other things, demonstrates that classifications provided herein can be correlated with pre-miRNA expression data and utilized for and/or included in assessments of subjects and/or their immune statuses and/or responsiveness to therapy.

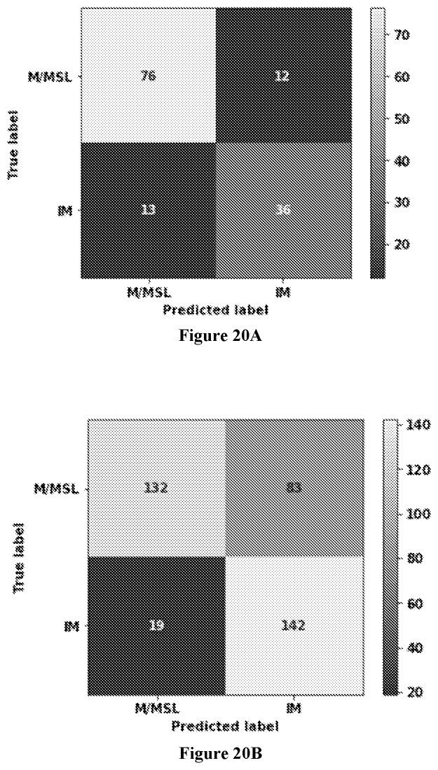

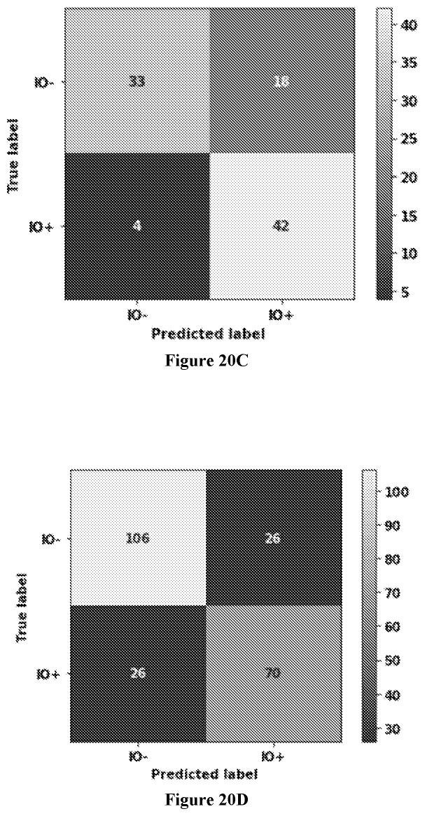

A list of pre-miRNAs was generated as outlined in Example 13 above (see Table 14). Expression levels of miRNAs of interest were used to build a linear regression model predicting continuous DetermaIO score. An optimal threshold was calculated using an f-1 score curve to see if such continuous scores could be mapped to a binary DetermaIO call. With a threshold of 021, a precision of 80% was achieved on the validation set and 75% for the blind test set when using expression of selected pre-miRNA to predict mRNA expression-based DetermaIO call (FIG. 20, parts A-B). The same process was repeated for the list of mature miRNAs previously described in Example 13 above (see Table 21B).

miRNAs of interest were also entered into the IntAct database from EMBL EBI. Results were filtered to show entries in which interation was between an miRNA of interest and genes of interest disclosed in the present application. Another filtering step narrowed the list to include either: 1) immune “cold” miRNAs (mapping to an M or MSL subtype) that interact with an immune “hot” gene (mapping to an IM subtype); or 2) immune “hot” miRNAs (mapping to an IM subtype) that interact with an immune “cold” gene (mapping to an M or MSL subtype). Resulting miRNAs of interest are outlined in Table 39 below.

TABLE 39
Hot and cold miRNA/gene interaction pairs.

| Alias(es) | Alias(es) | |
|---|---|---|
| interactor A | interactor B | Interaction type(s) |
| hsa-mir-29a- | mrna_igf1 | psi-mi: “MI: 0915”(physical association) |
| 3p | | |
| mrna_igf1 | hsa-mir-142-5p | psi-mi: “MI: 0915”(physical association) |
| mrna_zeb2 | hsa-mir-142-3p | psi-mi: “MI: 0915”(physical association) |
| mrna_zeb2 | hsa-mir-142-3p | psi-mi: “MI: 0915”(physical association) |
| hsa-mir-155- | mrna_zeb2 | psi-mi: “MI: 0915”(physical association) |
| 5p | | |

In some embodiments, a tumor sample (e.g., obtained through liquid biopsy, tissue biopsy, etc.) may be assessed to determine expression level of one or more classified pre-miRNAs as described herein. In some embodiments, treatments targeting or inhibiting miRNAs (e.g., pre-miRNAs, mature miRNAs, combinations thereof) classified under a different subtype as compared to one or more target genes could produce a shift in mRNA levels for said one or more target genes, resulting in changes in tumor signatures and/or IO scoring. For example, in some embodiments, treatment targeting or inhibiting miRNA (e.g., pre-miRNA, mature miRNA, combinations thereof) classified as one subtype (e.g., M, MSL) targeting a gene classified as a different subtype (e.g., IM) could produce an increase in overall IM signature for a tumor and result in an increased IO score. In some embodiments, treatment targeting or inhibiting miRNA (e.g., pre-miRNA, mature miRNA, combinations thereof) classified as one subtype (e.g., IM) targeting a gene classified as a different subtype (e.g., M, MSL) could produce an increase in overall M or MSL signature for a tumor and result in a decreased IO score. In some embodiments, information obtained from miRNA (e.g., pre-miRNA, mature miRNA, combinations thereof) assessment can be used to inform treatment decisions—e.g., selection and/or modification of therapy. In some particular embodiments, information obtained from miRNA (e.g., pre-miRNA, mature miRNA, combinations thereof) assessment can be used to inform selection and/or modification of combination therapies (e.g., additional therapy in combination with ICI therapy). In some embodiments, information obtained from matched data sets can be used to inform selection and/or modification of therapies, and in particular of combination therapies (e.g., additional therapy in combination with ICI therapy) based upon changes in IO scoring. In some embodiments, treatment targeting or inhibiting miRNA (e.g., pre-miRNA, mature miRNA, combinations thereof) could be combined with one or more therapies (e.g., chemotherapy, ICI, etc.) based upon changes in IO scoring.

### Example 20: Assessment of Interactions within Exemplary Gene Sets

The present Example, among other things, demonstrates that gene/gene interactions may be assessed within exemplary gene sets generated through methods disclosed herein in order to determine respnosiveness to therapy and/or select one or more therapies.

A set of genes generated through centroid modeling methods disclosed herein and in Example 11 were entered into the IntAct database from EMBL EBI. Results were filtered to generate a list of gene interactions and then further filtered to produce a list of interactions between an immune “hot” (IM) and immune “cold” (M, MSL) gene. These interactions are outlined in Table 40 below.

In some embodiments, gene interactions (e.g., between an immune “hot” and immune “cold” gene as described herein) may inform selection of one or more therapies. For example, in some embodiments, increased expression of an immune “cold” (M, MSL) gene with a known interaction with an immune “hot” (IM) gene may inform selection of one or more therapies intended to suppress the immune “cold” gene. In some embodiments, one or more therapies intended to supporess the immune “cold” gene may be combined with another therapy (e.g., ICI therapy). In some embodiments, increased expression of an immune “cold” (M, MSL) gene with a known interaction with an immune “hot” (IM) gene may be combined with DetermaIO scoring to inform selection of one or more therapies (e.g., ICI therapy).

TABLE 40
Hot and cold gene interaction pairs.

| Alias(es) | Alias(es) | A | B |
|---|---|---|---|
| interact | interact | Phe | Phe |
| or A | or B | no | no |
| FYB1 | FYN | Hot | Cold |
| ABL1 | HCK | Cold | Hot |
| PLCG1 | AGAP2 | Cold | Hot |
| HCK | ELMO1 | Hot | Cold |
| RCN2 | TRAF1 | Cold | Hot |
| GTF2I | NFKB2 | Cold | Hot |
| BCL7A | RELB | Cold | Hot |
| FYN | CD2 | Cold | Hot |
| CD2 | FYN | Hot | Cold |
| LEPR | JAK3 | Cold | Hot |
| LEPR | JAK2 | Cold | Hot |
| CLNK | FYN | Hot | Cold |
| LYN | EFS | Hot | Cold |
| ABL1 | DOK2 | Cold | Hot |
| DOK2 | ABL1 | Hot | Cold |
| SMAD9 | ASB2 | Cold | Hot |
| BTK | GTF2I | Hot | Cold |
| GTF2I | BTK | Cold | Hot |
| DLG4 | KCNA3 | Cold | Hot |
| KCNA3 | DLG4 | Hot | Cold |
| NCF4 | HDAC4 | Hot | Cold |
| MRC1 | BTK | Cold | Hot |
| CBX1 | BTK | Cold | Hot |
| EFS | LYN | Cold | Hot |
| CHD3 | PSME1 | Cold | Hot |
| BEND5 | GRAP2 | Cold | Hot |
| RBPMS | GRAP2 | Cold | Hot |
| TIFA | DVL2 | Hot | Cold |
| IFFO1 | ACAP1 | Cold | Hot |
| KCNJ4 | IL16 | Cold | Hot |
| GAS7 | FMNL1 | Cold | Hot |
| CSF1 | MYH10 | Hot | Cold |
| IKZF1 | PIAS3 | Hot | Cold |
| IKZF1 | ULK1 | Hot | Cold |
| FYN | HCLS1 | Cold | Hot |
| MX1 | TRPC6 | Hot | Cold |
| TRPC6 | MX1 | Cold | Hot |
| TRPC1 | MX1 | Cold | Hot |
| QKI | HCLS1 | Cold | Hot |
| SHC3 | DOK2 | Cold | Hot |
| PLCG1 | LCP2 | Cold | Hot |
| IGKC | CAV1 | Hot | Cold |
| WIPF1 | DLG4 | Hot | Cold |
| ENAH | ABI3 | Cold | Hot |
| IGHM | HBB | Hot | Cold |
| IGHG1 | LTBP4 | Hot | Cold |
| IGHG1 | ELN | Hot | Cold |
| IGHG1 | FGFR1 | Hot | Cold |
| MAX | EPAS1 | Hot | Cold |
| FYN | LAT | Cold | Hot |
| LAT | PLCG1 | Hot | Cold |
| TLR4 | LY96 | Cold | Hot |
| LY96 | TLR4 | Hot | Cold |
| ABL1 | ACAP1 | Cold | Hot |
| ABL1 | P2RX7 | Cold | Hot |
| ABL1 | FYB1 | Cold | Hot |
| WIPF1 | FYN | Hot | Cold |
| JAK2 | PLCG1 | Hot | Cold |
| AGO2 | ZBP1 | Cold | Hot |
| AGO2 | HERC5 | Cold | Hot |
| ARRB2 | MYH10 | Hot | Cold |
| ARRB2 | FLNA | Hot | Cold |
| ARRB2 | MAP1B | Hot | Cold |
| ARRB2 | SPIN1 | Hot | Cold |
| ARRB2 | BSN | Hot | Cold |
| FGF2 | CASP1 | Cold | Hot |
| STK4 | LATS2 | Hot | Cold |
| LATS2 | STK4 | Cold | Hot |
| PTPRC | TIE1 | Hot | Cold |
| GHR | PTPRC | Cold | Hot |
| PTPRC | LEPR | Hot | Cold |
| PTPRM | LCK | Cold | Hot |
| LCK | PTPRG | Hot | Cold |
| PTPRB | LCK | Cold | Hot |
| DLG4 | JAK3 | Cold | Hot |
| SLA2 | SYNM | Hot | Cold |
| AGAP2 | MPRIP | Hot | Cold |
| BTK | RFTN1 | Hot | Cold |
| LCP2 | FYN | Hot | Cold |
| PLEK | ACTN1 | Hot | Cold |
| HDAC5 | MAX | Cold | Hot |
| MMP9 | ERG | Hot | Cold |
| SVIL | MYO1G | Cold | Hot |
| ESR1 | LACTB | Cold | Hot |
| ESR2 | LACTB | Cold | Hot |
| ESR2 | ASB2 | Cold | Hot |
| FSTL1 | CD14 | Cold | Hot |
| PLCG1 | LAT | Cold | Hot |
| FNBP1 | ADAM8 | Cold | Hot |
| ADAM8 | SNX33 | Hot | Cold |
| GAB3 | DLG4 | Hot | Cold |
| DLG4 | IRF7 | Cold | Hot |
| SP100 | CBX5 | Hot | Cold |
| LDOC1 | IKZF3 | Cold | Hot |
| ADA | SGCD | Hot | Cold |
| CRYAB | CCL22 | Cold | Hot |
| P2RX7 | EFNB3 | Hot | Cold |
| IRF2 | FKBP7 | Hot | Cold |
| IL23A | ESR2 | Hot | Cold |
| GFI1 | PIAS3 | Hot | Cold |
| PIAS3 | GFI1 | Cold | Hot |
| LCP2 | PLCG1 | Hot | Cold |
| GRIP1 | AGAP2 | Cold | Hot |
| AGAP2 | GRIA1 | Hot | Cold |
| AGAP2 | GRIP1 | Hot | Cold |
| CD14 | FSTL1 | Hot | Cold |
| DHX58 | AGO2 | Hot | Cold |
| AGO2 | DHX58 | Cold | Hot |
| FMNL1 | FMNL3 | Hot | Cold |
| FZD8 | C1QA | Cold | Hot |
| C1QA | FZD1 | Hot | Cold |
| C1QA | FZD7 | Hot | Cold |
| C1QA | FZD8 | Hot | Cold |
| C1QA | FZD4 | Hot | Cold |
| VCAM1 | TLN1 | Hot | Cold |
| VCAM1 | FLNA | Hot | Cold |
| VCAM1 | FLNC | Hot | Cold |
| VCAM1 | QKI | Hot | Cold |
| VCAM1 | HIP1 | Hot | Cold |
| VCAM1 | MYH10 | Hot | Cold |
| FYB1 | PLCG1 | Hot | Cold |
| WIPF1 | ROBO4 | Hot | Cold |
| FUT8 | GCH1 | Cold | Hot |
| STAT1 | KIT | Hot | Cold |
| PLCG1 | HCK | Cold | Hot |
| TEAD3 | FAS | Cold | Hot |
| DOCK8 | LRCH2 | Hot | Cold |
| CBX1 | MAX | Cold | Hot |
| ARRB2 | SMO | Hot | Cold |
| RUNX3 | HDAC4 | Hot | Cold |
| RUNX3 | HDAC5 | Hot | Cold |
| HDAC4 | RUNX3 | Cold | Hot |
| HDAC5 | RUNX3 | Cold | Hot |
| AGO2 | HERC6 | Cold | Hot |
| ESR2 | IL24 | Cold | Hot |
| CAV1 | NCF1 | Cold | Hot |
| LCK | KIT | Hot | Cold |
| KIT | ZAP70 | Cold | Hot |
| KIT | FGR | Cold | Hot |
| HSH2D | KIT | Hot | Cold |
| KIT | HCK | Cold | Hot |
| STAP1 | KIT | Hot | Cold |
| KIT | LCK | Cold | Hot |
| KIT | HSH2D | Cold | Hot |
| LYN | KIT | Hot | Cold |
| TXK | KIT | Hot | Cold |
| BLK | KIT | Hot | Cold |
| KIT | LYN | Cold | Hot |
| GRAP2 | KIT | Hot | Cold |
| HCK | KIT | Hot | Cold |
| KIT | GRAP2 | Cold | Hot |
| ZAP70 | KIT | Hot | Cold |
| CLNK | PTK7 | Hot | Cold |
| MPEG1 | TNS2 | Hot | Cold |
| STK4 | ACACA | Hot | Cold |
| STK4 | PAK3 | Hot | Cold |
| STK4 | GTF2I | Hot | Cold |
| WIPF1 | ABI2 | Hot | Cold |
| MEOX2 | STX11 | Cold | Hot |
| LZTS2 | LCK | Cold | Hot |
| ATF7 | FCER2 | Cold | Hot |
| AQP1 | TRAF1 | Cold | Hot |
| IKZF3 | AQP1 | Hot | Cold |
| BLK | EFS | Hot | Cold |
| TCL1A | TXLNB | Hot | Cold |
| RTP5 | BEND5 | Hot | Cold |
| RBPMS | RTP5 | Cold | Hot |
| LZTS2 | RTP5 | Cold | Hot |
| LPXN | TXLNB | Hot | Cold |
| NMI | TXLNB | Hot | Cold |
| TXLNB | NMI | Cold | Hot |
| ABI3 | KANK2 | Hot | Cold |
| DOK3 | RBPMS | Hot | Cold |
| NATD1 | IKZF3 | Cold | Hot |
| DOCK8 | MEOX2 | Hot | Cold |
| LMO3 | SAMD3 | Cold | Hot |
| IKZF1 | LMO3 | Hot | Cold |
| TNIP3 | LZTS2 | Hot | Cold |
| PILRA | PIANP | Hot | Cold |
| SNX33 | ADAM8 | Cold | Hot |
| BEND5 | RTP5 | Cold | Hot |
| TXLNB | LPXN | Cold | Hot |
| KANK2 | ABI3 | Cold | Hot |
| RBPMS | DOK3 | Cold | Hot |
| MEOX2 | DOCK8 | Cold | Hot |
| SAMD3 | LMO3 | Hot | Cold |
| RBPMS | FOXP3 | Cold | Hot |
| CARD9 | MEOX2 | Hot | Cold |
| MEOX2 | GPSM3 | Cold | Hot |
| TRAF1 | AQP1 | Hot | Cold |
| RTP5 | RBPMS | Hot | Cold |
| LZTS2 | CARD9 | Cold | Hot |
| ESR1 | NFKB2 | Cold | Hot |
| ESR1 | FAS | Cold | Hot |
| ESR2 | NFKB2 | Cold | Hot |
| AIM2 | RCN2 | Hot | Cold |
| AIM2 | FOXC1 | Hot | Cold |
| AIM2 | GTF2I | Hot | Cold |
| AIM2 | BEND3 | Hot | Cold |
| MNDA | RCN2 | Hot | Cold |
| MNDA | DHX57 | Hot | Cold |
| ESR1 | STAT1 | Cold | Hot |
| FOXP3 | NOVA2 | Hot | Cold |
| FOXP3 | BEND3 | Hot | Cold |
| FOXP3 | NOVA1 | Hot | Cold |
| FOXP3 | FOXP2 | Hot | Cold |
| MAX | WIZ | Hot | Cold |
| FOXP3 | FOXP1 | Hot | Cold |
| FGD1 | TAP2 | Cold | Hot |
| MYH10 | LYN | Cold | Hot |
| FYCO1 | DDX60 | Cold | Hot |
| NLRP3 | CBX5 | Hot | Cold |
| FOXO1 | FOXP3 | Cold | Hot |
| NINL | ARRB2 | Cold | Hot |
| NINL | XRN1 | Cold | Hot |
| IL10 | MEF2D | Hot | Cold |
| MAF | IL10 | Cold | Hot |
| PLCG1 | CD244 | Cold | Hot |
| CD244 | PLCG1 | Hot | Cold |
| RAB37 | RAB3C | Hot | Cold |
| LATS2 | GRAP2 | Cold | Hot |
| DVL2 | GRAP2 | Cold | Hot |
| DVL2 | TIFA | Cold | Hot |
| GRAP2 | LATS2 | Hot | Cold |
| GRAP2 | DVL2 | Hot | Cold |
| MEOX1 | PARVG | Cold | Hot |
| LPXN | TNS2 | Hot | Cold |
| FOSB | FOXP3 | Cold | Hot |
| TRAF1 | CRY2 | Hot | Cold |
| GPR25 | VAPB | Hot | Cold |
| AXIN2 | STX11 | Cold | Hot |
| DLG2 | AOAH | Cold | Hot |
| IKZF3 | HSPB7 | Hot | Cold |
| CD53 | CD302 | Hot | Cold |
| RPRM | FGD2 | Cold | Hot |
| STX11 | SHC3 | Hot | Cold |
| TIFA | KANK2 | Hot | Cold |
| CD33 | RPRM | Hot | Cold |
| FPR2 | FXYD6 | Hot | Cold |
| CD79A | APOD | Hot | Cold |
| IL21R | AQP1 | Hot | Cold |
| CD53 | CNIH2 | Hot | Cold |
| RHOH | KANK2 | Hot | Cold |
| CD53 | RPRM | Hot | Cold |
| CD79A | AQP1 | Hot | Cold |
| CD79A | VAPB | Hot | Cold |
| CD79A | CNIH3 | Hot | Cold |
| CD79A | WFS1 | Hot | Cold |
| CD53 | APOD | Hot | Cold |
| SUSD3 | CNIH3 | Hot | Cold |
| BACH2 | BATF3 | Cold | Hot |
| IKZF3 | KANK2 | Hot | Cold |
| TRAF1 | KANK2 | Hot | Cold |
| TRAF1 | SHC3 | Hot | Cold |
| LZTS2 | TNIP3 | Cold | Hot |
| KCNA1 | APOL2 | Cold | Hot |
| KLHL6 | CRY2 | Hot | Cold |
| LPXN | AQP1 | Hot | Cold |
| CD69 | RPRM | Hot | Cold |
| ABI3 | NHSL2 | Hot | Cold |
| KCNA3 | KCNA1 | Hot | Cold |
| ISLR2 | NKG7 | Cold | Hot |
| SUSD3 | RPRM | Hot | Cold |
| FPR2 | APOD | Hot | Cold |
| CD79A | HACD4 | Hot | Cold |
| SIT1 | ITM2A | Hot | Cold |
| VAPB | GPR25 | Cold | Hot |
| CD302 | CD53 | Cold | Hot |
| PRKN | FAS | Cold | Hot |
| LRP1 | APOE | Cold | Hot |
| RPRM | CD33 | Cold | Hot |
| FXYD6 | FPR2 | Cold | Hot |
| CNIH2 | CD53 | Cold | Hot |
| KANK2 | RHOH | Cold | Hot |
| RPRM | CD53 | Cold | Hot |
| RPRM | SUSD3 | Cold | Hot |
| KIF3C | STK4 | Cold | Hot |
| SMAP2 | SMAD9 | Hot | Cold |
| MEOX2 | BTK | Cold | Hot |
| LAT | GRAP | Hot | Cold |
| LAT | TLN1 | Hot | Cold |
| LAT | FYN | Hot | Cold |
| LCP2 | GRAP | Hot | Cold |
| CD36 | TLR6 | Cold | Hot |
| TLR4 | TLR6 | Cold | Hot |
| CD36 | LYN | Cold | Hot |
| C1QA | HSPB2 | Hot | Cold |
| KCNA5 | KCNA3 | Cold | Hot |
| LSP1 | TMOD1 | Hot | Cold |
| BATF3 | RTL6 | Hot | Cold |
| BATF3 | NPTX2 | Hot | Cold |
| CD79A | TTC28 | Hot | Cold |
| LAMP3 | SYNM | Hot | Cold |
| LAMP3 | ARL10 | Hot | Cold |
| ICAM1 | FOXF1 | Hot | Cold |
| ICAM1 | TTC28 | Hot | Cold |
| CYTIP | HBB | Hot | Cold |
| STX11 | KIF7 | Hot | Cold |
| CARD8 | SALL2 | Hot | Cold |
| CARD8 | PEAK1 | Hot | Cold |
| CARD8 | LRCH2 | Hot | Cold |
| BIN2 | AMPH | Hot | Cold |
| CD1B | NRSN2 | Hot | Cold |
| VNN2 | GPM6A | Hot | Cold |
| VNN2 | APOD | Hot | Cold |
| VNN2 | FZD7 | Hot | Cold |
| HTRA4 | TTC28 | Hot | Cold |
| MMRN1 | APOE | Cold | Hot |
| LPXN | LPP | Hot | Cold |
| LPXN | MIB1 | Hot | Cold |
| LPXN | CEP68 | Hot | Cold |
| FGL2 | FUT8 | Hot | Cold |
| FGL2 | KIF7 | Hot | Cold |
| ESR1 | GBP1 | Cold | Hot |
| XCL1 | FSTL1 | Hot | Cold |
| ICAM3 | FUT8 | Hot | Cold |
| S1PR1 | CD69 | Cold | Hot |
| FLNA | ARRB2 | Cold | Hot |
| PLCG1 | ITK | Cold | Hot |
| ARRB2 | EDNRA | Hot | Cold |
| EDNRA | ARRB2 | Cold | Hot |
| TAL1 | LYL1 | Cold | Hot |
| JAK2 | LEPR | Hot | Cold |
| FNBP1 | WIPF1 | Cold | Hot |
| AQP1 | CD47 | Cold | Hot |
| CCL22 | CCL14 | Hot | Cold |
| ISG15 | SYNM | Hot | Cold |
| DLG4 | AGAP2 | Cold | Hot |
| AGAP2 | DLG4 | Hot | Cold |
| AGAP2 | NUMBL | Hot | Cold |
| AGAP2 | ANK2 | Hot | Cold |
| AGAP2 | SOGA1 | Hot | Cold |
| AGAP2 | ACTN1 | Hot | Cold |
| AGAP2 | VAPB | Hot | Cold |
| AGAP2 | TLN2 | Hot | Cold |
| AGAP2 | DCLK1 | Hot | Cold |
| AGAP2 | MAP6 | Hot | Cold |
| AGAP2 | ENAH | Hot | Cold |
| IFIH1 | WIZ | Hot | Cold |
| AGTR1 | ABI3 | Cold | Hot |
| AGTR1 | CD37 | Cold | Hot |
| CCR4 | RTN1 | Hot | Cold |
| AGTR1 | ARRB2 | Cold | Hot |
| NCBP3 | BST2 | Cold | Hot |
| NCBP3 | CSF1 | Cold | Hot |
| FYN | FLT3 | Cold | Hot |
| ABL1 | FLT3 | Cold | Hot |
| APOE | LRP1 | Hot | Cold |
| DDX60 | RCN2 | Hot | Cold |
| DDX60 | SYNC | Hot | Cold |
| IRF7 | MAP1B | Hot | Cold |
| MX1 | LRP4 | Hot | Cold |
| NOD2 | MYL9 | Hot | Cold |
| NOD2 | MYL3 | Hot | Cold |
| OAS3 | TLN1 | Hot | Cold |
| OASL | DHX57 | Hot | Cold |
| VAMP5 | SYNPO | Hot | Cold |
| APBB1 | MEI1 | Cold | Hot |
| APBB1 | WDFY4 | Cold | Hot |
| DTNA | PLCB2 | Cold | Hot |
| ESR1 | XRN1 | Cold | Hot |
| PIAS3 | APOE | Cold | Hot |
| STK4 | FYN | Hot | Cold |
| LAT | ITPR1 | Hot | Cold |
| PTPRC | CASQ2 | Hot | Cold |
| PTPRC | PLCG1 | Hot | Cold |
| EPHB3 | CD14 | Cold | Hot |
| SH2D2A | GTF2I | Hot | Cold |
| ITK | PLCG1 | Hot | Cold |
| SYDE1 | LYN | Cold | Hot |
| STAP1 | CDO1 | Hot | Cold |
| STAT1 | LMOD1 | Hot | Cold |
| TXK | CDO1 | Hot | Cold |
| ZAP70 | CDO1 | Hot | Cold |
| SOCS1 | RCN2 | Hot | Cold |
| HCK | CHD3 | Hot | Cold |
| DOK2 | FLNA | Hot | Cold |
| DOK2 | SPIN1 | Hot | Cold |
| LRCH2 | DOCK8 | Cold | Hot |
| FOXP3 | ZEB2 | Hot | Cold |
| HTRA1 | HTRA4 | Cold | Hot |
| HTRA4 | TGFB3 | Hot | Cold |
| WFS1 | BATF | Cold | Hot |
| WFS1 | GPSM3 | Cold | Hot |
| WFS1 | TLR10 | Cold | Hot |
| NCBP3 | PSME1 | Cold | Hot |
| GLIS2 | IL27 | Cold | Hot |
| KLF2 | IL32 | Cold | Hot |
| GLIS2 | IL32 | Cold | Hot |
| MAX | TNFSF12 | Hot | Cold |
| TIFAB | ACTN1 | Hot | Cold |
| HDAC4 | ABI3 | Cold | Hot |
| HDAC5 | FMNL1 | Cold | Hot |
| CNR2 | RCN2 | Hot | Cold |
| PI16 | TRPV2 | Cold | Hot |
| ABL1 | TCL1A | Cold | Hot |
| FGR | APOD | Hot | Cold |
| LATS2 | HERC6 | Cold | Hot |
| CPEB1 | XRN1 | Cold | Hot |
| CPEB1 | SMAP2 | Cold | Hot |
| IRF1 | BCL7A | Hot | Cold |
| IRF4 | CIC | Hot | Cold |
| IRF4 | BCL7A | Hot | Cold |
| IRF4 | MEF2D | Hot | Cold |
| IRF8 | CIC | Hot | Cold |
| IRF8 | BCL9 | Hot | Cold |
| IRF8 | FLI1 | Hot | Cold |
| KLF8 | MAX | Cold | Hot |
| STAT4 | ALMS1 | Hot | Cold |
| STAT4 | BCL7A | Hot | Cold |
| FGFR1 | SMAP2 | Cold | Hot |
| SKAP1 | FYN | Hot | Cold |
| CTLA4 | FYN | Hot | Cold |
| CIQA | LRP1 | Hot | Cold |
| ADCY2 | ARRB2 | Cold | Hot |
| STK4 | MAP1B | Hot | Cold |
| BTK | FLNA | Hot | Cold |
| BTK | ACTN1 | Hot | Cold |
| IFIT2 | RCN2 | Hot | Cold |
| DLG4 | SYTL3 | Cold | Hot |
| HCLS1 | DLG4 | Hot | Cold |
| RUNX3 | DLG4 | Hot | Cold |
| FRMD6 | IKZF1 | Cold | Hot |
| FOXP3 | RBPMS | Hot | Cold |
| MEOX2 | CARD9 | Cold | Hot |
| CARD9 | LZTS2 | Hot | Cold |
| PARVG | MEOX2 | Hot | Cold |
| GPSM3 | MEOX2 | Hot | Cold |
| ABI2 | WIPF1 | Cold | Hot |
| STX11 | MEOX2 | Hot | Cold |
| LCK | LZTS2 | Hot | Cold |
| AQP1 | IKZF3 | Cold | Hot |
| TXLNB | TCL1A | Cold | Hot |
| PARVG | MEOX1 | Hot | Cold |
| TNS2 | LPXN | Cold | Hot |
| FOXP3 | FOSB | Hot | Cold |
| CRY2 | TRAF1 | Cold | Hot |
| BATF3 | BACH2 | Hot | Cold |
| KANK2 | TRAF1 | Cold | Hot |
| SHC3 | TRAF1 | Cold | Hot |
| AQP1 | LPXN | Cold | Hot |
| RPRM | CD69 | Cold | Hot |
| KCNA1 | KCNA3 | Cold | Hot |
| SHC3 | STX11 | Cold | Hot |
| CNR2 | CNR1 | Hot | Cold |
| CNR1 | CNR2 | Cold | Hot |
| ESR1 | PREX1 | Cold | Hot |

### Example 21: Assessment of Tumor Immune Infiltrate Signatures

The present Example, among other things, demonstrates that classifications provided herein can be correlated with tumor immune infiltrate types and may be utilized and/or included in assessments of subjects and/or their immune statuses and/or responsiveness to therapy.

### Preparation of RNASeq Datasets

Gene expression data sets from tumors in twenty tissue types (lung squamous cell, lung adenocarcinoma, breast, ovarian, kidney clear cell, head and neck, prostate, melanoma, colon, bladder, pancreas, kidney papillary, sarcoma, rectal, B cell lymphoma, kidney chromophobe, esophageal, stomach cancers, thymoma, and acute myeloid leukemia) were downloaded from the US NIH Genomic Data Commons Portal (https://portal.gdc.cancer.gov/). Additionally, murine RNAseq data from the ImmGen projects GSE109125 (Yoshida, Lareau et al. 2019) and GSE122108 (ImmGen 2016) were downloaded. The first dataset, GSE109125, are primary RNAseq data for 103 highly purified immunocyte populations representing all lineages and several differentiation cascades, profiled using the ImmGen ULI pipeline. The second dataset, GSE122108 are primary RNASeq data for progenitor, resident, and stimulated (C.alb, LPS, injury, APAP+ starved overnight and pIC) mononuclear phagocytes from fourteen organs.

Each tumor's gene expression data was processed identically. Experiment data was converted to a DGE list using the R (version 4.1.1) function SE2DGEList in the edgeR package (Robinson, McCarthy et al. 2010). Duplicate genes were reduced to a single gene by selecting the gene with the maximum standard deviation across all samples in the data set. Mouse gene names were converted to human orthologs using biomaRt (Durinck, Spellman et al. 2009). When there was gene duplication in orthologs, the gene with greatest homology with a human ortholog was retained. HUGO Gene Nomenclature Committee (HGNC) names for each gene were added using the biomarRt package. Genes without a HGNC designation were removed from the analysis. Using TMM normalization in the EdgeR package, normalization factors were calculated, applied, and log 2 transformed counts per million (cpm) were determined for each gene for each sample. The distribution of cpm and the standard deviation of genes across samples was plotted, and cutoffs determined to remove very low expressing genes. Samples were then renormalized using TMM after these genes were removed.

Potential confounding effects due to the institution from which a tumor sample was acquired was addressed by removing batch effect based on the tissue source site using the EdgeR package. To combine the two ImmGen datasets, each was batch corrected in edgeR, but finding no differences in models created using batch corrected and unaltered expression data, no batch correction was employed for final models using ImmGen data. The normalized and batch corrected gene expression human data sets were randomly combined into a 2/3 training, 1/3 test set with tumors balanced for tumor type between the two sets, without shared institutions between the two. This resulted in 4696 tumor samples in the training set, and 2195 samples in the test set. This gene expression data set is referred to herein as TCGA20.

### Creation of ImmGen-Derived Signatures

One set of immune cell signatures was created from defined mouse immune cell populations. Gene expression data from the ImmGen public consortium (www.immgen.org) was downloaded. These data are derived from defined and purified mouse cells using the ImmGen ULI GSE109125 and GSE122108 data sets. Gene names were translated into human homologs, and when duplicate genes were present, those with maximum standard deviation across all lines were selected. To allow the applicability of models created using the ImmGen data to human tumors, this list of genes was limited to those that were present in both the TCGA20 and ImmGen dataset (N=14,611). Genes were further limited to those that Ensembl had scored as having a high confidence for the orthology (mmusculus homolog orthology confidence=1), resulting in 14094 genes. ImmGen cell populations were grouped according to cell type, and also subdivided based on tissue of origin to identify potentially novel molecularly physiologically defined subtypes via k-means clustering, with k being determined with factoextra (Kassambara and Mundt 2020) using the elbow method (within total sum of squares). An elastic net (Friedman, Hastie et al. 2010) was used with lambda being set so that models contain at least five genes (unbounded on the upper end). The selected genes and coefficients for these models are shown in Table 41A.

TABLE 41A

| Model | Model | Gene | Gene |
|---|---|---|---|
| names | description | Names | coefficients |
| abT | alpha beta T cells | (Intercept) | 2.001435055 |
| abT | alpha beta T cells | AGFG2 | 0.02817566 |
| abT | alpha beta T cells | ARHGEF11 | −0.127029452 |
| abT | alpha beta T cells | ATF6 | −0.036109757 |
| abT | alpha beta T cells | BBC3 | −0.046892288 |
| abT | alpha beta T cells | CCDC186 | −0.277005348 |
| abT | alpha beta T cells | DNAAF4 | 0.010442018 |
| abT | alpha beta T cells | DRC7 | 0.228172052 |
| abT | alpha beta T cells | EGLN2 | −0.025400659 |
| abT | alpha beta T cells | ESM1 | 0.126208896 |
| abT | alpha beta T cells | FBXO4 | −0.020497817 |
| abT | alpha beta T cells | HDAC9 | −0.12708357 |
| abT | alpha beta T cells | IL2RA | 0.003309983 |
| abT | alpha beta T cells | KCNH2 | 0.162758024 |
| abT | alpha beta T cells | KLC3 | 0.139619461 |
| abT | alpha beta T cells | LYPD6B | 0.128050897 |
| abT | alpha beta T cells | MC1R | 0.065291376 |
| abT | alpha beta T cells | NCMAP | 0.307146288 |
| abT | alpha beta T cells | PLEKHA7 | 0.160165673 |
| abT | alpha beta T cells | SOX13 | −0.028158057 |
| abT | alpha beta T cells | TEDDM1 | 0.000519815 |
| abT | alpha beta T cells | TMEM106B | −0.376396054 |
| abT | alpha beta T cells | TRAT1 | 0.104222021 |
| abT | alpha beta T cells | TRAV22 | 0.320717609 |
| abT | alpha beta T cells | TRBV12-4 | 0.158582519 |
| abT | alpha beta T cells | TRBV28 | 0.603361788 |
| abT | alpha beta T cells | TRBV30 | 0.255161162 |
| abT | alpha beta T cells | TRIM26 | −0.091774153 |
| abT | alpha beta T cells | TRPA1 | 0.134336354 |
| abT | alpha beta T cells | WFIKKN2 | −0.100872871 |
| abT | alpha beta T cells | WNK3 | −0.014812825 |
| abT | alpha beta T cells | ZNF516 | −0.246461708 |
| B_mature | mature B cells | (Intercept) | −1.549992694 |
| B_mature | mature B cells | ATP11A | −0.031583511 |
| B_mature | mature B cells | CACNA1I | 0.063664894 |
| B_mature | mature B cells | KLHL14 | 0.535385558 |
| B_mature | mature B cells | LCP2 | −0.50433392 |
| CD8.T.act | CD8+ activated T | (Intercept) | −6.64893008 |
| | cells | | |
| CD8.T.act | CD8+ activated T | GZMM | 0.080056183 |
| | cells | | |
| CD8.T.act | CD8+ activated T | LRRC75B | 0.362673397 |
| | cells | | |
| CD8.T.act | CD8+ activated T | MMP16 | 0.086408591 |
| | cells | | |
| CD8.T.act | CD8+ activated T | NCMAP | −0.045303327 |
| | cells | | |
| CD8.T.act | CD8+ activated T | OSGIN1 | −0.096222092 |
| | cells | | |
| CD8.T.act | CD8+ activated T | TRAV23DV6 | 0.712614542 |
| | cells | | |
| CD8.T.act | CD8+ activated T | WFIKKN2 | 0.335981436 |
| | cells | | |
| Dendritic_ | Dendritic cells, k- | (Intercept) | −8.123768812 |
| clus1 | means defined | | |
| | cluster 1 | | |
| Dendritic_ | Dendritic cells, k- | ACTR1B | 0.158306819 |
| clus1 | means defined | | |
| | cluster 1 | | |
| Dendritic_ | Dendritic cells, k- | CADM3 | 0.023449502 |
| clus1 | means defined | | |
| | cluster 1 | | |
| Dendritic_ | Dendritic cells, k- | CCDC170 | 0.126059806 |
| clus1 | means defined | | |
| | cluster 1 | | |
| Dendritic_ | Dendritic cells, k- | DKK2 | 0.285146848 |
| clus1 | means defined | | |
| | cluster 1 | | |
| Dendritic_ | Dendritic cells, k- | MMP12 | 0.033976559 |
| clus1 | means defined | | |
| | cluster 1 | | |
| Dendritic_ | Dendritic cells, k- | NCCRP1 | 0.447996595 |
| clus1 | means defined | | |
| | cluster 1 | | |
| Dendritic_ | Dendritic cells, k- | PNPO | 0.110169671 |
| clus1 | means defined | | |
| | cluster 1 | | |
| Dendritic_ | Dendritic cells, k- | PPP1R14A | 0.252866799 |
| clus1 | means defined | | |
| | cluster 1 | | |
| Dendritic_ | Dendritic cells, k- | RADIL | 0.062270484 |
| clus1 | means defined | | |
| | cluster 1 | | |
| Dendritic_ | Dendritic cells, k- | STK32C | 0.019624646 |
| clus1 | means defined | | |
| | cluster 1 | | |
| Dendritic_ | Dendritic cells, k- | SUCNR1 | 0.407838462 |
| clus1 | means defined | | |
| | cluster 1 | | |
| Dendritic_ | Dendritic cells, k- | TRAPPC5 | 0.008898636 |
| clus1 | means defined | | |
| | cluster 1 | | |
| Dendritic_ | Dendritic cells, k- | VRK1 | 0.386902817 |
| clus1 | means defined | | |
| | cluster 1 | | |
| Dendritic_ | Dendritic cells, k- | (Intercept) | −6.722646121 |
| clus2 | means defined | | |
| | cluster 2 | | |
| Dendritic_ | Dendritic cells, k- | CLTRN | 0.568477717 |
| clus2 | means defined | | |
| | cluster 2 | | |
| Dendritic_ | Dendritic cells, k- | DDX58 | −0.03843886 |
| clus2 | means defined | | |
| | cluster 2 | | |
| Dendritic_ | Dendritic cells, k- | GRAMD2A | 0.153602144 |
| clus2 | means defined | | |
| | cluster 2 | | |
| Dendritic_ | Dendritic cells, k- | PPT1 | 0.459595114 |
| clus2 | means defined | | |
| | cluster 2 | | |
| Dendritic_ | Dendritic cells, k- | WLS | −0.119007461 |
| clus2 | means defined | | |
| | cluster 2 | | |
| Dendritic_ | Dendritic cells, k- | XCR1 | 0.050767932 |
| clus2 | means defined | | |
| | cluster 2 | | |
| Dendritic_ | Dendritic cells, k- | (Intercept) | −13.3434289 |
| clus3 | means defined | | |
| | cluster 3 | | |
| Dendritic_ | Dendritic cells, k- | CEBPZ | −0.043224663 |
| clus3 | means defined | | |
| | cluster 3 | | |
| Dendritic_ | Dendritic cells, k- | DAO | 0.12683647 |
| clus3 | means defined | | |
| | cluster 3 | | |
| Dendritic_ | Dendritic cells, k- | DCAF12 | −0.121365297 |
| clus3 | means defined | | |
| | cluster 3 | | |
| Dendritic_ | Dendritic cells, k- | EHMT1 | 0.213071073 |
| clus3 | means defined | | |
| | cluster 3 | | |
| Dendritic_ | Dendritic cells, k- | EIF4G3 | −0.241703538 |
| clus3 | means defined | | |
| | cluster 3 | | |
| Dendritic_ | Dendritic cells, k- | MMP7 | 0.390350102 |
| clus3 | means defined | | |
| | cluster 3 | | |
| Dendritic_ | Dendritic cells, k- | OPRD1 | 0.119991091 |
| clus3 | means defined | | |
| | cluster 3 | | |
| Dendritic_ | Dendritic cells, k- | POLR3C | 1.084677505 |
| clus3 | means defined | | |
| | cluster 3 | | |
| Dendritic_ | Dendritic cells, k- | RGS6 | 0.188014525 |
| clus3 | means defined | | |
| | cluster 3 | | |
| Dendritic_ | Dendritic cells, k- | TBC1D17 | 0.289201175 |
| clus3 | means defined | | |
| | cluster 3 | | |
| Dendritic_ | Dendritic cells, k- | (Intercept) | −13.5669761 |
| clus 4 | means defined | | |
| | cluster 4 | | |
| Dendritic_ | Dendritic cells, k- | BTBD1 | −0.702940517 |
| clus 4 | means defined | | |
| | cluster 4 | | |
| Dendritic_ | Dendritic cells, k- | ELANE | 0.311312019 |
| clus 4 | means defined | | |
| | cluster 4 | | |
| Dendritic_ | Dendritic cells, k- | GALM | −0.008124744 |
| clus 4 | means defined | | |
| | cluster 4 | | |
| Dendritic_ | Dendritic cells, k- | GTDC1 | −0.283430504 |
| clus 4 | means defined | | |
| | cluster 4 | | |
| Dendritic_ | Dendritic cells, k- | KAT7 | 0.070669481 |
| clus 4 | means defined | | |
| | cluster 4 | | |
| Dendritic_ | Dendritic cells, k- | MPO | 0.065416945 |
| clus 4 | means defined | | |
| | cluster 4 | | |
| Dendritic_ | Dendritic cells, k- | NKX2-3 | 0.004093534 |
| clus 4 | means defined | | |
| | cluster 4 | | |
| Dendritic_ | Dendritic cells, k- | PAQR5 | 0.063290853 |
| clus 4 | means defined | | |
| | cluster 4 | | |
| Dendritic_ | Dendritic cells, k- | PLA2G4D | 0.553801138 |
| clus 4 | means defined | | |
| | cluster 4 | | |
| Dendritic_ | Dendritic cells, k- | PNCK | 0.334293496 |
| clus 4 | means defined | | |
| | cluster 4 | | |
| Dendritic_ | Dendritic cells, k- | RAB33B | 0.370419827 |
| clus 4 | means defined | | |
| | cluster 4 | | |
| Dendritic_ | Dendritic cells, k- | RGP1 | 0.444839022 |
| clus 4 | means defined | | |
| | cluster 4 | | |
| Dendritic_ | Dendritic cells, k- | RNF11 | −0.263831387 |
| clus 4 | means defined | | |
| | cluster 4 | | |
| Dendritic_ | Dendritic cells, k- | RNF220 | 0.006810057 |
| clus 4 | means defined | | |
| | cluster 4 | | |
| Dendritic_ | Dendritic cells, k- | RNU4-2 | 0.063610658 |
| clus 4 | means defined | | |
| | cluster 4 | | |
| Dendritic_ | Dendritic cells, k- | SLC34A1 | 0.013380526 |
| clus 4 | means defined | | |
| | cluster 4 | | |
| Dendritic_ | Dendritic cells, k- | TEX2 | 0.036354183 |
| clus 4 | means defined | | |
| | cluster 4 | | |
| Dendritic_ | Dendritic cells, k- | THEM6 | 0.063757031 |
| clus 4 | means defined | | |
| | cluster 4 | | |
| Dendritic_ | Dendritic cells, k- | TRAPPC5 | 1.033001367 |
| clus 4 | means defined | | |
| | cluster 4 | | |
| Dendritic_ | Dendritic cells, k- | TRIM58 | 0.031600281 |
| clus 4 | means defined | | |
| | cluster 4 | | |
| Dendritic_ | Dendritic cells, k- | UPB1 | 0.205840822 |
| clus 4 | means defined | | |
| | cluster 4 | | |
| Dendritic_ | Dendritic cells, k- | ZDHHC19 | 0.471118953 |
| clus 4 | means defined | | |
| | cluster 4 | | |
| gdT | gamma-delta T | (Intercept) | −6.017483799 |
| | cells | | |
| gdT | gamma-delta T | CCR10 | 0.097500198 |
| | cells | | |
| gdT | gamma-delta T | DAPL1 | 0.078407795 |
| | cells | | |
| gdT | gamma-delta T | FAM124B | 0.122009728 |
| | cells | | |
| gdT | gamma-delta T | GGT1 | 0.0534037 |
| | cells | | |
| gdT | gamma-delta T | SOX13 | 0.460115544 |
| | cells | | |
| gdT | gamma-delta T | TRDC | 0.38370397 |
| | cells | | |
| ILC | Innate lymphoid | (Intercept) | −3.616471171 |
| | cells | | |
| ILC | Innate lymphoid | CRYGN | 0.130466746 |
| | cells | | |
| ILC | Innate lymphoid | KLHL30 | 1.46E−05 |
| | cells | | |
| ILC | Innate lymphoid | KLRG1 | 0.025191909 |
| | cells | | |
| ILC | Innate lymphoid | MATK | −0.180422579 |
| | cells | | |
| ILC | Innate lymphoid | NCR1 | 0.375227391 |
| | cells | | |
| ILC | Innate lymphoid | NMUR1 | 0.448198899 |
| | cells | | |
| ILC | Innate lymphoid | PHACTR3 | 0.174855253 |
| | cells | | |
| ILC | Innate lymphoid | RASGEF1C | 0.003713342 |
| | cells | | |
| ILC | Innate lymphoid | RET | 0.132813605 |
| | cells | | |
| ILC | Innate lymphoid | SCARB1 | −0.07105525 |
| | cells | | |
| ILC | Innate lymphoid | SPRY2 | 0.013616034 |
| | cells | | |
| Macrophage_ | Macrophage cells, | (Intercept) | −7.091174516 |
| clus1 | k-means defined | | |
| | cluster 1 | | |
| Macrophage_ | Macrophage cells, | ADAMDEC1 | 0.082406686 |
| clus1 | k-means defined | | |
| | cluster 1 | | |
| Macrophage_ | Macrophage cells, | ANKDD1A | −0.120502141 |
| clus1 | k-means defined | | |
| | cluster 1 | | |
| Macrophage_ | Macrophage cells, | B3GNT7 | 0.02882706 |
| clus1 | k-means defined | | |
| | cluster 1 | | |
| Macrophage_ | Macrophage cells, | FAM83F | 0.096395206 |
| clus1 | k-means defined | | |
| | cluster 1 | | |
| Macrophage_ | Macrophage cells, | HAMP | −0.03965109 |
| clus1 | k-means defined | | |
| | cluster 1 | | |
| Macrophage_ | Macrophage cells, | IGF2BP3 | 0.056557041 |
| clus1 | k-means defined | | |
| | cluster 1 | | |
| Macrophage_ | Macrophage cells, | KCNJ10 | 0.01181564 |
| clus1 | k-means defined | | |
| | cluster 1 | | |
| Macrophage_ | Macrophage cells, | LGI4 | 0.036759845 |
| clus1 | k-means defined | | |
| | cluster 1 | | |
| Macrophage_ | Macrophage cells, | MATN3 | 0.026126966 |
| clus1 | k-means defined | | |
| | cluster 1 | | |
| Macrophage_ | Macrophage cells, | MMP12 | 0.08377652 |
| clus1 | k-means defined | | |
| | cluster 1 | | |
| Macrophage_ | Macrophage cells, | MMP13 | 0.268521879 |
| clus1 | k-means defined | | |
| | cluster 1 | | |
| Macrophage_ | Macrophage cells, | PARP9 | 0.065091506 |
| clus1 | k-means defined | | |
| | cluster 1 | | |
| Macrophage_ | Macrophage cells, | RAG1 | 0.046479459 |
| clus1 | k-means defined | | |
| | cluster 1 | | |
| Macrophage_ | Macrophage cells, | SLC13A3 | 0.046205156 |
| clus1 | k-means defined | | |
| | cluster 1 | | |
| Macrophage_ | Macrophage cells, | SLC6A1 | −0.006575066 |
| clus1 | k-means defined | | |
| | cluster 1 | | |
| Macrophage_ | Macrophage cells, | SPON1 | 0.087465459 |
| clus1 | k-means defined | | |
| | cluster 1 | | |
| Macrophage_ | Macrophage cells, | TCF21 | 0.259197858 |
| clus1 | k-means defined | | |
| | cluster 1 | | |
| Macrophage_ | Macrophage cells, | TMEM117 | −0.032205423 |
| clus1 | k-means defined | | |
| | cluster 1 | | |
| Macrophage_ | Macrophage cells, | TRIM55 | 0.268894639 |
| clus1 | k-means defined | | |
| | cluster 1 | | |
| Macrophage_ | Macrophage cells, | TSPAN10 | 0.135826007 |
| clus1 | k-means defined | | |
| | cluster 1 | | |
| Macrophage_ | Macrophage cells, | VIPAS39 | 0.291509288 |
| clus1 | k-means defined | | |
| | cluster 1 | | |
| Macrophage_ | Macrophage cells, | ZFYVE28 | 0.096370959 |
| clus1 | k-means defined | | |
| | cluster 1 | | |
| Macrophage_ | Macrophage cells, | (Intercept) | −4.020385943 |
| clus2 | k-means defined | | |
| | cluster 2 | | |
| Macrophage_ | Macrophage cells, | C6 | 0.248733891 |
| clus2 | k-means defined | | |
| | cluster 2 | | |
| Macrophage_ | Macrophage cells, | DYSF | 0.215674654 |
| clus2 | k-means defined | | |
| | cluster 2 | | |
| Macrophage_ | Macrophage cells, | FGG | 0.095031229 |
| clus2 | k-means defined | | |
| | cluster 2 | | |
| Macrophage_ | Macrophage cells, | PCOLCE2 | 0.055627283 |
| clus2 | k-means defined | | |
| | cluster 2 | | |
| Macrophage_ | Macrophage cells, | STAB2 | 0.030343236 |
| clus2 | k-means defined | | |
| | cluster 2 | | |
| Macrophage_ | Macrophage cells, | (Intercept) | −8.230602674 |
| clus3 | k-means defined | | |
| | cluster 3 | | |
| Macrophage_ | Macrophage cells, | HES2 | 0.577907806 |
| clus3 | k-means defined | | |
| | cluster 3 | | |
| Macrophage_ | Macrophage cells, | HIBCH | −0.061966381 |
| clus3 | k-means defined | | |
| | cluster 3 | | |
| Macrophage_ | Macrophage cells, | HLA-DRA | 0.054458451 |
| clus3 | k-means defined | | |
| | cluster 3 | | |
| Macrophage_ | Macrophage cells, | KLK13 | 0.052526618 |
| clus3 | k-means defined | | |
| | cluster 3 | | |
| Macrophage_ | Macrophage cells, | LGI3 | 0.550751383 |
| clus3 | k-means defined | | |
| | cluster 3 | | |
| Macrophage_ | Macrophage cells, | RBPJL | 0.676278283 |
| clus3 | k-means defined | | |
| | cluster 3 | | |
| Macrophage_ | Macrophage cells, | RTBDN | 0.020583144 |
| clus3 | k-means defined | | |
| | cluster 3 | | |
| Macrophage_ | Macrophage cells, | SAP18 | 0.474280619 |
| clus3 | k-means defined | | |
| | cluster 3 | | |
| Macrophage_ | Macrophage cells, | TCOF1 | 0.335241139 |
| clus3 | k-means defined | | |
| | cluster 3 | | |
| Macrophage_ | Macrophage cells, | (Intercept) | −3.729871533 |
| clus4 | k-means defined | | |
| | cluster 4 | | |
| Macrophage_ | Macrophage cells, | CALHM2 | −0.038894611 |
| clus4 | k-means defined | | |
| | cluster 4 | | |
| Macrophage_ | Macrophage cells, | CTSK | 0.15724825 |
| clus4 | k-means defined | | |
| | cluster 4 | | |
| Macrophage_ | Macrophage cells, | DUSP13 | 0.377946852 |
| clus4 | k-means defined | | |
| | cluster 4 | | |
| Macrophage_ | Macrophage cells, | RCSD1 | −0.0752708 |
| clus4 | k-means defined | | |
| | cluster 4 | | |
| Macrophage_ | Macrophage cells, | TRIM29 | 0.024329186 |
| clus4 | k-means defined | | |
| | cluster 4 | | |
| Macrophage_ | Macrophage cells, | ZFAND2A | 0.30151853 |
| clus4 | k-means defined | | |
| | cluster 4 | | |
| Macrophage_ | Macrophage cells, | (Intercept) | −7.12929125 |
| clus5 | k-means defined | | |
| | cluster 5 | | |
| Macrophage_ | Macrophage cells, | CALML4 | 0.103286223 |
| clus5 | k-means defined | | |
| | cluster 5 | | |
| Macrophage_ | Macrophage cells, | CLRN3 | 0.017322042 |
| clus5 | k-means defined | | |
| | cluster 5 | | |
| Macrophage_ | Macrophage cells, | DTX3 | −0.140238486 |
| clus5 | k-means defined | | |
| | cluster 5 | | |
| Macrophage_ | Macrophage cells, | GATA6 | 0.39415529 |
| clus5 | k-means defined | | |
| | cluster 5 | | |
| Macrophage_ | Macrophage cells, | ICAM2 | 0.050052326 |
| clus5 | k-means defined | | |
| | cluster 5 | | |
| Macrophage_ | Macrophage cells, | PFKL | 0.023916193 |
| clus5 | k-means defined | | |
| | cluster 5 | | |
| Macrophage_ | Macrophage cells, | PRG4 | 0.110299091 |
| clus5 | k-means defined | | |
| | cluster 5 | | |
| Macrophage_ | Macrophage cells, | PYCARD | 0.287196787 |
| clus5 | k-means defined | | |
| | cluster 5 | | |
| Macrophage_ | Macrophage cells, | RP1 | 0.173075061 |
| clus5 | k-means defined | | |
| | cluster 5 | | |
| Macrophage_ | Macrophage cells, | SELP | 0.055773841 |
| clus5 | k-means defined | | |
| | cluster 5 | | |
| Macrophage_ | Macrophage cells, | WNT2 | 0.100552962 |
| clus5 | k-means defined | | |
| | cluster 5 | | |
| Macrophage_ | Macrophage cells, | (Intercept) | −4.132570306 |
| clus6 | k-means defined | | |
| | cluster 6 | | |
| Macrophage_ | Macrophage cells, | ADAMDEC1 | −0.008257374 |
| clus6 | k-means defined | | |
| | cluster 6 | | |
| Macrophage_ | Macrophage cells, | ANAPC7 | 0.105478953 |
| clus6 | k-means defined | | |
| | cluster 6 | | |
| Macrophage_ | Macrophage cells, | AP1B1 | 0.232559156 |
| clus6 | k-means defined | | |
| | cluster 6 | | |
| Macrophage_ | Macrophage cells, | CD207 | −0.002113765 |
| clus6 | k-means defined | | |
| | cluster 6 | | |
| Macrophage_ | Macrophage cells, | CDKL4 | −0.056586152 |
| clus6 | k-means defined | | |
| | cluster 6 | | |
| Macrophage_ | Macrophage cells, | CLCF1 | −0.044286212 |
| clus6 | k-means defined | | |
| | cluster 6 | | |
| Macrophage_ | Macrophage cells, | DENND3 | −0.106884529 |
| clus6 | k-means defined | | |
| | cluster 6 | | |
| Macrophage_ | Macrophage cells, | DOT1L | −0.044799992 |
| clus6 | k-means defined | | |
| | cluster 6 | | |
| Macrophage_ | Macrophage cells, k- | GGCT | −0.146509 |
| clus6 | means defined cluster 6 | | |
| Macrophage_ | Macrophage cells, k- | HELZ2 | −0.141925 |
| clus6 | means defined cluster 6 | | |
| Macrophage_ | Macrophage cells, k- | HS3ST3B1 | −0.082309 |
| clus6 | means defined cluster 6 | | |
| Macrophage_ | Macrophage cells, k- | KLHL33 | 0.1253497 |
| clus6 | means defined cluster 6 | | |
| Macrophage_ | Macrophage cells, k- | LMTK2 | −0.474807 |
| clus6 | means defined cluster 6 | | |
| Macrophage_ | Macrophage cells, k- | MOG | 0.0144396 |
| clus6 | means defined cluster 6 | | |
| Macrophage_ | Macrophage cells, k- | NOL10 | −0.00831 |
| clus6 | means defined cluster 6 | | |
| Macrophage_ | Macrophage cells, k- | PHYKPL | −0.208104 |
| clus6 | means defined cluster 6 | | |
| Macrophage_ | Macrophage cells, k- | PTK2B | −0.22175 |
| clus6 | means defined cluster 6 | | |
| Macrophage_ | Macrophage cells, k- | REPS2 | 0.0618522 |
| clus6 | means defined cluster 6 | | |
| Macrophage_ | Macrophage cells, k- | SH3GL1 | 0.2562942 |
| clus6 | means defined cluster 6 | | |
| Macrophage_ | Macrophage cells, k- | SMPD3 | 0.0281673 |
| clus6 | means defined cluster 6 | | |
| Macrophage_ | Macrophage cells, k- | SNX6 | 0.0488675 |
| clus6 | means defined cluster 6 | | |
| Macrophage_ | Macrophage cells, k- | SORBS1 | −0.112206 |
| clus6 | means defined cluster 6 | | |
| Macrophage_ | Macrophage cells, k- | STK40 | −0.277894 |
| clus6 | means defined cluster 6 | | |
| Macrophage_ | Macrophage cells, k- | USP16 | −0.128092 |
| clus6 | means defined cluster 6 | | |
| Macrophage_ | Macrophage cells, k- | WWP1 | 0.559664 |
| clus6 | means defined cluster 6 | | |
| Macrophage_ | Macrophage cells, k- | (Intercept) | −0.842026 |
| clus7 | means defined cluster 7 | | |
| Macrophage_ | Macrophage cells, k- | ANKRD12 | −0.918137 |
| clus7 | means defined cluster 7 | | |
| Macrophage_ | Macrophage cells, k- | ATP13A5 | −0.264776 |
| clus7 | means defined cluster 7 | | |
| Macrophage_ | Macrophage cells, k- | CAPN1 | −0.105234 |
| clus7 | means defined cluster 7 | | |
| Macrophage_ | Macrophage cells, k- | CDC26 | 0.1433384 |
| clus7 | means defined cluster 7 | | |
| Macrophage_ | Macrophage cells, k- | FOXRED2 | −0.010795 |
| clus7 | means defined cluster 7 | | |
| Macrophage_ | Macrophage cells, k- | H4C14 | −0.034928 |
| clus7 | means defined cluster 7 | | |
| Macrophage_ | Macrophage cells, k- | HAPLN1 | 0.1492342 |
| clus7 | means defined cluster 7 | | |
| Macrophage_ | Macrophage cells, k- | HPCAL4 | 0.0032812 |
| clus7 | means defined cluster 7 | | |
| Macrophage_ | Macrophage cells, k- | HSPB3 | 0.6597222 |
| clus7 | means defined cluster 7 | | |
| Macrophage_ | Macrophage cells, k- | KLF2 | −0.043163 |
| clus7 | means defined cluster 7 | | |
| Macrophage_ | Macrophage cells, k- | MTDH | 0.3174562 |
| clus7 | means defined cluster 7 | | |
| Macrophage_ | Macrophage cells, k- | PLEKHG3 | −0.273146 |
| clus7 | means defined cluster 7 | | |
| Macrophage_ | Macrophage cells, k- | SNAP25 | 0.1833946 |
| clus7 | means defined cluster 7 | | |
| Macrophage_ | Macrophage cells, k- | SPOCK2 | −0.05739 |
| clus7 | means defined cluster 7 | | |
| Macrophage_ | Macrophage cells, k- | USP53 | −0.04631 |
| clus7 | means defined cluster 7 | | |
| Mast_cell | Mast cells | (Intercept) | −6.320001 |
| Mast_cell | Mast cells | SMPX | 0.115906 |
| Mast_cell | Mast cells | STK32B | 0.3220007 |
| Mast_cell | Mast cells | TPSAB1 | 0.0001682 |
| Mast_cell | Mast cells | TPSB2 | 1.60E−15 |
| Mast_cell | Mast cells | TPSG1 | 0.7737122 |
| Microglia | Microglial cells | (Intercept) | −3.274518 |
| Microglia | Microglial cells | ATP13A5 | 0.0254463 |
| Microglia | Microglial cells | COX14 | −0.155755 |
| Microglia | Microglial cells | COX6B1 | −0.487811 |
| Microglia | Microglial cells | CSMD3 | 0.3769117 |
| Microglia | Microglial cells | MEIG1 | 0.0293387 |
| Microglia | Microglial cells | PFN1 | −0.152983 |
| Microglia | Microglial cells | SLCO1C1 | 0.0746896 |
| Microglia | Microglial cells | TMX4 | 0.5382676 |
| Monocyte_ | Monocyte cells, k- | (Intercept) | −12.3884 |
| clus1 | means defined cluster 1 | | |
| Monocyte_ | Monocyte cells, k- | ACE | 0.4953719 |
| clus1 | means defined cluster 1 | | |
| Monocyte_ | Monocyte cells, k- | ADAM32 | −0.130983 |
| clus1 | means defined cluster 1 | | |
| Monocyte_ | Monocyte cells, k- | ADAMTS4 | −0.118824 |
| clus1 | means defined cluster 1 | | |
| Monocyte_ | Monocyte cells, k- | ALB | 0.1326397 |
| clus1 | means defined cluster 1 | | |
| Monocyte_ | Monocyte cells, k- | C3orf20 | −0.043665 |
| clus1 | means defined cluster 1 | | |
| Monocyte_ | Monocyte cells, k- | CCDC68 | 0.1930406 |
| clus1 | means defined cluster 1 | | |
| Monocyte_ | Monocyte cells, k- | CTNNA3 | −0.081262 |
| clus1 | means defined cluster 1 | | |
| Monocyte_ | Monocyte cells, k- | CXCL11 | 0.0169017 |
| clus1 | means defined cluster 1 | | |
| Monocyte_ | Monocyte cells, k- | CXCL9 | 0.0945416 |
| clus1 | means defined cluster 1 | | |
| Monocyte_ | Monocyte cells, k- | CYP1A1 | 0.0094036 |
| clus1 | means defined cluster 1 | | |
| Monocyte_ | Monocyte cells, k- | DNAH6 | −0.117278 |
| clus1 | means defined cluster 1 | | |
| Monocyte_ | Monocyte cells, k- | ENPP6 | −0.018847 |
| clus1 | means defined cluster 1 | | |
| Monocyte_ | Monocyte cells, k- | ETHE1 | 0.2915961 |
| clus1 | means defined cluster 1 | | |
| Monocyte_ | Monocyte cells, k- | FCN1 | 0.0807263 |
| clus1 | means defined cluster 1 | | |
| Monocyte_ | Monocyte cells, k- | FLRT3 | −0.073566 |
| clus1 | means defined cluster 1 | | |
| Monocyte_ | Monocyte cells, k- | FUT9 | −0.204966 |
| clus1 | means defined cluster 1 | | |
| Monocyte_ | Monocyte cells, k- | GDF15 | 0.0934752 |
| clus1 | means defined cluster 1 | | |
| Monocyte_ | Monocyte cells, k- | GDF3 | 0.189272 |
| clus1 | means defined cluster 1 | | |
| Monocyte_ | Monocyte cells, k- | GLP1R | 0.3059683 |
| clus1 | means defined cluster 1 | | |
| Monocyte_ | Monocyte cells, k- | GPNMB | 0.1573053 |
| clus1 | means defined cluster 1 | | |
| Monocyte_ | Monocyte cells, k- | KLK1 | 0.0809401 |
| clus1 | means defined cluster 1 | | |
| Monocyte_ | Monocyte cells, k- | LNX1 | −0.080215 |
| clus1 | means defined cluster 1 | | |
| Monocyte_ | Monocyte cells, k- | MROH2A | −0.013603 |
| clus1 | means defined cluster 1 | | |
| Monocyte_ | Monocyte cells, k- | MYRF | −0.025713 |
| clus1 | means defined cluster 1 | | |
| Monocyte_ | Monocyte cells, k- | NR0B2 | 0.0356621 |
| clus1 | means defined cluster 1 | | |
| Monocyte_ | Monocyte cells, k- | PTPRB | 0.3180536 |
| clus1 | means defined cluster 1 | | |
| Monocyte_ | Monocyte cells, k- | RNF222 | −0.015217 |
| clus1 | means defined cluster 1 | | |
| Monocyte_ | Monocyte cells, k- | SDK2 | −0.378768 |
| clus1 | means defined cluster 1 | | |
| Monocyte_ | Monocyte cells, k- | SLC1A2 | −0.065051 |
| clus1 | means defined cluster 1 | | |
| Monocyte_ | Monocyte cells, k- | SYNGR1 | 0.0108365 |
| clus1 | means defined cluster 1 | | |
| Monocyte_ | Monocyte cells, k- | TMEM117 | −0.080936 |
| clus1 | means defined cluster 1 | | |
| Monocyte_ | Monocyte cells, k- | VIPAS39 | 0.4075701 |
| clus1 | means defined cluster 1 | | |
| Monocyte_ | Monocyte cells, k- | (Intercept) | −10.00118 |
| clus2 | means defined cluster 2 | | |
| Monocyte_ | Monocyte cells, k- | CAPRIN2 | −0.120379 |
| clus2 | means defined cluster 2 | | |
| Monocyte_ | Monocyte cells, k- | CENPE | −0.112678 |
| clus2 | means defined cluster 2 | | |
| Monocyte_ | Monocyte cells, k- | CXCL11 | 0.0059509 |
| clus2 | means defined cluster 2 | | |
| Monocyte_ | Monocyte cells, k- | GPLD1 | 0.1435982 |
| clus2 | means defined cluster 2 | | |
| Monocyte_ | Monocyte cells, k- | HIF1A | 0.4876146 |
| culs2 | means defined cluster 2 | | |
| Monocyte_ | Monocyte cells, k- | HLA-DRA | −0.006791 |
| clus2 | means defined cluster 2 | | |
| Monocyte_ | Monocyte cells, k- | HOPX | 0.0075312 |
| clus2 | means defined cluster 2 | | |
| Monocyte_ | Monocyte cells, k- | IMMP2L | −0.016049 |
| clus2 | means defined cluster 2 | | |
| Monocyte_ | Monocyte cells, k- | JADE2 | −0.091665 |
| clus2 | means defined cluster 2 | | |
| Monocyte_ | Monocyte cells, k- | KYAT1 | −0.258265 |
| clus2 | means defined cluster 2 | | |
| Monocyte_ | Monocyte cells, k- | LSM11 | −0.026337 |
| clus2 | means defined cluster 2 | | |
| Monocyte_ | Monocyte cells, k- | MBLAC2 | −0.190036 |
| clus2 | means defined cluster 2 | | |
| Monocyte_ | Monocyte cells, k- | MPIG6B | 0.0296941 |
| clus2 | means defined cluster 2 | | |
| Monocyte_ | Monocyte cells, k- | MRNIP | −0.076447 |
| clus2 | means defined cluster 2 | | |
| Monocyte_ | Monocyte cells, k- | PCID2 | 0.426335 |
| clus2 | means defined cluster 2 | | |
| Monocyte_ | Monocyte cells, k- | PTCH1 | −0.214745 |
| clus2 | means defined cluster 2 | | |
| Monocyte_ | Monocyte cells, k- | RAD9B | −0.229406 |
| clus2 | means defined cluster 2 | | |
| Monocyte_ | Monocyte cells, k- | REST | 0.4973285 |
| clus2 | means defined cluster 2 | | |
| Monocyte_ | Monocyte cells, k- | RHOV | 0.0636676 |
| clus2 | means defined cluster 2 | | |
| Monocyte_ | Monocyte cells, k- | SH2D6 | 0.3383157 |
| clus2 | means defined cluster 2 | | |
| Monocyte_ | Monocyte cells, k- | TM4SF19 | 0.113478 |
| clus2 | means defined cluster 2 | | |
| Monocyte_ | Monocyte cells, k- | TM4SF19- | 1.71E−17 |
| clus2 | means defined cluster 2 | DYNLT2B | |
| Monocyte_ | Monocyte cells, k- | ULK1 | −0.000917 |
| clus2 | means defined cluster 2 | | |
| Monocyte_ | Monocyte cells, k- | (Intercept) | −4.785432 |
| clus3 | means defined cluster 3 | | |
| Monocyte_ | Monocyte cells, k- | CCDC192 | 0.3610516 |
| clus3 | means defined cluster 3 | | |
| Monocyte_ | Monocyte cells, k- | SCNN1A | 0.1586076 |
| clus3 | means defined cluster 3 | | |
| Monocyte_ | Monocyte cells, k- | SCNN1G | 0.0844571 |
| clus3 | means defined cluster 3 | | |
| Monocyte_ | Monocyte cells, k- | TGM3 | 0.1254089 |
| clus3 | means defined cluster 3 | | |
| Monocyte_ | Monocyte cells, k- | TMEM121B | 0.6782325 |
| clus3 | means defined cluster 3 | | |
| Monocyte_ | Monocyte cells, k- | (Intercept) | −3.234572 |
| clus4 | means defined cluster 4 | | |
| Monocyte_ | Monocyte cells, k- | C1GALT1C1 | 0.2737209 |
| clus4 | means defined cluster 4 | | |
| Monocyte_ | Monocyte cells, k- | CD9 | −0.051805 |
| clus4 | means defined cluster 4 | | |
| Monocyte_ | Monocyte cells, k- | DCST2 | 0.01848 |
| clus4 | means defined cluster 4 | | |
| Monocyte_ | Monocyte cells, k- | DIABLO | −0.370062 |
| clus4 | means defined cluster 4 | | |
| Monocyte_ | Monocyte cells, k- | GOT1 | −0.213832 |
| clus4 | means defined cluster 4 | | |
| Monocyte_ | Monocyte cells, k- | JADE2 | −0.034674 |
| clus4 | means defined cluster 4 | | |
| Monocyte_ | Monocyte cells, k- | MGAT4A | −0.218986 |
| clus4 | means defined cluster 4 | | |
| Monocyte_ | Monocyte cells, k- | RBKS | 0.5059444 |
| ucls4 | means defined cluster 4 | | |
| Monocyte_ | Monocyte cells, k- | RSPO1 | 0.5938048 |
| clus4 | means defined cluster 4 | | |
| Monocyte_ | Monocyte cells, k- | TSTD1 | 0.0312869 |
| clus4 | means defined cluster 4 | | |
| stem | stem cells | (Intercept) | −5.451469 |
| stem | stem cells | DACH1 | 0.0523615 |
| stem | stem cells | HOXA10 | 0.0228399 |
| stem | stem cells | MAP7D2 | 0.4619375 |
| stem | stem cells | MPL | 0.2202485 |
| stem | stem cells | NKX2-3 | 0.734782 |
| stem | stem cells | SAMD12 | 0.0350193 |
| stroma | stromal cells | (Intercept) | −4.394228 |
| stroma | stromal cells | IGFBP5 | 0.2713573 |
| stroma | stromal cells | MSX1 | 0.2815415 |
| stroma | stromal cells | PRSS23 | 0.0936651 |
| stroma | stromal cells | RGS4 | 0.1496425 |
| stroma | stromal cells | STC1 | 0.1310073 |
| stroma | stromal cells | TNC | 0.0550913 |
| stroma | stromal cells | UBD | 0.1262248 |
| Dendritic_ | Dendritic cells | (Intercept) | −5.912319 |
| cell_all | | | |
| Dendritic_ | Dendritic cells | ADAM11 | 0.1002379 |
| cell_all | | | |
| Dendritic_ | Dendritic cells | AP1S3 | 0.0214533 |
| cell_all | | | |
| Dendritic_ | Dendritic cells | BCL2L14 | 0.0092591 |
| cell_all | | | |
| Dendritic_ | Dendritic cells | BLOC1S2 | 0.6153238 |
| cell_all | | | |
| Dendritic_ | Dendritic cells | CACNG8 | 0.1962639 |
| cell_all | | | |
| Dendritic_ | Dendritic cells | CD84 | −0.056769 |
| cell_all | | | |
| Dendritic_ | Dendritic cells | CNTRL | 0.0341237 |
| cell_all | | | |
| Dendritic_ | Dendritic cells | COL23A1 | 0.0634118 |
| cell_all | | | |
| Dendritic_ | Dendritic cells | DDX58 | −0.006258 |
| cell_all | | | |
| Dendritic_ | Dendritic cells | FFAR4 | 0.0680295 |
| cell_all | | | |
| Dendritic_ | Dendritic cells | FLT3 | 0.0678801 |
| cell_all | | | |
| Dendritic_ | Dendritic cells | GPR4 | 0.0187906 |
| cell_all | | | |
| Dendritic_ | Dendritic cells | LPAR3 | 0.0846475 |
| cell_all | | | |
| Dendritic_ | Dendritic cells | MTMR14 | 0.036754 |
| cell_all | | | |
| Dendritic_ | Dendritic cells | MYCL | 0.0313354 |
| cell_all | | | |
| Dendritic_ | Dendritic cells | NCCRP1 | 0.069873 |
| cell_all | | | |
| Dendritic_ | Dendritic cells | NME9 | 0.0015251 |
| cell_all | | | |
| Dendritic_ | Dendritic cells | POLG | −0.122899 |
| cell_all | | | |
| Dendritic_ | Dendritic cells | SH3BP5 | −0.081743 |
| cell_all | | | |
| Dendritic_ | Dendritic cells | ZBTB46 | 0.0753025 |
| cell_all | | | |
| Dendritic_ | Dendritic cells | ZMIZ2 | 0.1389079 |
| cell_all | | | |
| granulocyte | Granulocytes | (Intercept) | −1.573921 |
| granulocyte | Granulocytes | BPNT1 | −0.398936 |
| granulocyte | Granulocytes | CKLF | 0.0768094 |
| granulocyte | Granulocytes | CLCN3 | −0.005616 |
| granulocyte | Granulocytes | CLPB | −0.150694 |
| granulocyte | Granulocytes | EDNRA | 0.1639499 |
| granulocyte | Granulocytes | MS4A2 | 0.2927978 |
| granulocyte | Granulocytes | NCAM1 | 0.1325591 |
| granulocyte | Granulocytes | NLRP12 | 0.390913 |
| granulocyte | Granulocytes | SLC25A4 | −0.09173 |
| granulocyte | Granulocytes | SLC41A1 | −0.053435 |
| granulocyte | Granulocytes | STK32B | 0.2356176 |
| granulocyte | Granulocytes | TMEM40 | 0.0298787 |
| granulocyte | Granulocytes | ZNF146 | −0.153386 |
| Macrophage_ | Macrophage cells with | (Intercept) | −4.106222 |
| Brain | brain tissue origins | | |
| Macrophage_ | Macrophage cells with | AKAP3 | 0.0374379 |
| Brain | brain tissue origins | | |
| Macrophage_ | Macrophage cells with | ARR3 | −0.017346 |
| Brain | brain tissue origins | | |
| Macrophage_ | Macrophage cells with | ASB16 | −0.009873 |
| Brain | brain tissue origins | | |
| Macrophage_ | Macrophage cells with | CLGN | 0.0509409 |
| Brain | brain tissue origins | | |
| Macrophage_ | Macrophage cells with | EBF4 | −0.021489 |
| Brain | brain tissue origins | | |
| Macrophage_ | Macrophage cells with | EFCAB6 | −0.188585 |
| Brain | brain tissue origins | | |
| Macrophage_ | Macrophage cells with | ENAM | −0.068888 |
| Brain | brain tissue origins | | |
| Macrophage_ | Macrophage cells with | HPCAL4 | 0.1597102 |
| Brain | brain tissue origins | | |
| Macrophage_ | Macrophage cells with | HSPB3 | 1.3420763 |
| Brain | brain tissue origins | | |
| Macrophage_ | Macrophage cells with | KLF2 | −0.236661 |
| Brain | brain tissue origins | | |
| Macrophage_ | Macrophage cells with | MEIG1 | −0.274868 |
| Brain | brain tissue origins | | |
| Macrophage_ | Macrophage cells with | QRFPR | 0.3079325 |
| Brain | brain tissue origins | | |
| Macrophage_ | Macrophage cells with | SALL1 | 0.0265053 |
| Brain | brain tissue origins | | |
| Macrophage_ | Macrophage cells with | SLCO1C1 | −0.073116 |
| Brain | brain tissue origins | | |
| Macrophage_ | Macrophage cells with | SNAP25 | 0.0906954 |
| Brain | brain tissue origins | | |
| Macrophage_ | Macrophage cells with | SPOCK2 | −0.161885 |
| Brain | brain tissue origins | | |
| Macrophage_ | Macrophage cells with | TAGLN3 | 0.0898985 |
| Brain | brain tissue origins | | |
| Macrophage_ | Macrophage cells with | VTN | −0.157918 |
| Brain | brain tissue origins | | |
| Macrophage_ | Macrophage cells with | (Intercept) | −3.391014 |
| Heart | heart tissue origins | | |
| Macrophage_ | Macrophage cells with | ACTC1 | 0.1507811 |
| Heart | heart tissue origins | | |
| Macrophage_ | Macrophage cells with | AREG | 0.0735382 |
| Heart | heart tissue origins | | |
| Macrophage_ | Macrophage cells with | FOSL1 | 0.044466 |
| Heart | heart tissue origins | | |
| Macrophage_ | Macrophage cells with | HLA-DRA | 0.2742392 |
| Heart | heart tissue origins | | |
| Macrophage_ | Macrophage cells with | LSMEM1 | 0.0824488 |
| Heart | heart tissue origins | | |
| Macrophage_ | Macrophage cells with | TNNC1 | 0.3733761 |
| Heart | heart tissue origins | | |
| Macrophage_ | Macrophage cells with | (Intercept) | −5.754201 |
| Liver | liver tissue origins | | |
| Macrophage_ | Macrophage cells with | AASS | 0.5996662 |
| Liver | liver tissue origins | | |
| Macrophage_ | Macrophage cells with | C6 | 0.1840223 |
| Liver | liver tissue origins | | |
| Macrophage_ | Macrophage cells with | CD207 | 0.0004984 |
| Liver | liver tissue origins | | |
| Macrophage_ | Macrophage cells with | CDH5 | 0.1824303 |
| Liver | liver tissue origins | | |
| Macrophage_ | Macrophage cells with | CLEC4G | 0.0398063 |
| Liver | liver tissue origins | | |
| Macrophage_ | Macrophage cells with | FGG | 0.3530157 |
| Liver | liver tissue origins | | |
| Macrophage_ | Macrophage cells with | SERPINA1 | 0.2808324 |
| Liver | liver tissue origins | | |
| Macrophage_ | Macrophage cells with | SMC1B | 0.2803812 |
| Liver | liver tissue origins | | |
| Macrophage_ | Macrophage cells with | (Intercept) | 0.3288313 |
| Lung | lung tissue origins | | |
| Macrophage_ | Macrophage cells with | B3GNT7 | 0.1271161 |
| Lung | lung tissue origins | | |
| Macrophage_ | Macrophage cells with | CHD5 | 0.0546691 |
| Lung | lung tissue origins | | |
| Macrophage_ | Macrophage cells with | FLVCR2 | 0.1739783 |
| Lung | lung tissue origins | | |
| Macrophage_ | Macrophage cells with | ITPR3 | −0.156732 |
| Lung | lung tissue origins | | |
| Macrophage_ | Macrophage cells with | KLHL33 | 0.2293481 |
| Lung | lung tissue origins | | |
| Macrophage_ | Macrophage cells with | LBH | −0.349301 |
| Lung | lung tissue origins | | |
| Macrophage_ | Macrophage cells with | MFAP4 | 0.2702648 |
| Lung | lung tissue origins | | |
| Macrophage_ | Macrophage cells with | MIDN | −0.204931 |
| Lung | lung tissue origins | | |
| Macrophage_ | Macrophage cells with | MISP3 | 0.0084275 |
| Lung | lung tissue origins | | |
| Macrophage_ | Macrophage cells with | NSMAF | −0.178247 |
| Lung | lung tissue origins | | |
| Macrophage_ | Macrophage cells with | SLC9A4 | 0.035116 |
| Lung | lung tissue origins | | |
| Macrophage_ | Macrophage cells with | TBX4 | 0.5009861 |
| Lung | lung tissue origins | | |
| Macrophage_ | Macrophage cells with | (Intercept) | −10.86262 |
| Other | other tissue origins | | |
| Macrophage_ | Macrophage cells with | A4GALT | 0.3428367 |
| Other | other tissue origins | | |
| Macrophage_ | Macrophage cells with | AAR2 | 0.1886536 |
| Other | other tissue origins | | |
| Macrophage_ | Macrophage cells with | AKT3 | −0.052303 |
| Other | other tissue origins | | |
| Macrophage_ | Macrophage cells with | ASGR2 | 0.1333548 |
| Other | other tissue origins | | |
| Macrophage_ | Macrophage cells with | C3orf62 | −0.068357 |
| Other | other tissue origins | | |
| Macrophage_ | Macrophage cells with | CFD | 0.2517181 |
| Other | other tissue origins | | |
| Macrophage_ | Macrophage cells with | DLST | 0.583357 |
| Other | other tissue origins | | |
| Macrophage_ | Macrophage cells with | EPOR | 0.1387225 |
| Other | other tissue origins | | |
| Macrophage_ | Macrophage cells with | FAM83G | −0.01287 |
| Other | other tissue origins | | |
| Macrophage_ | Macrophage cells with | GPC4 | 0.0370189 |
| Other | other tissue origins | | |
| Macrophage_ | Macrophage cells with | GSK3A | −0.198529 |
| Other | other tissue origins | | |
| Macrophage_ | Macrophage cells with | IGF2BP1 | 0.2902343 |
| Other | other tissue origins | | |
| Macrophage_ | Macrophage cells with | IGF2BP3 | 0.003376 |
| Other | other tissue origins | | |
| Macrophage_ | Macrophage cells with | IL4I1 | −0.027645 |
| Other | other tissue origins | | |
| Macrophage_ | Macrophage cells with | LDB3 | −0.046662 |
| Other | other tissue origins | | |
| Macrophage_ | Macrophage cells with | LIMCH1 | −0.027317 |
| Other | other tissue origins | | |
| Macrophage_ | Macrophage cells with | LRAT | −0.250784 |
| Other | other tissue origins | | |
| Macrophage_ | Macrophage cells with | MMP13 | 0.1846006 |
| Other | other tissue origins | | |
| Macrophage_ | Macrophage cells with | NEXN | −0.098988 |
| Other | other tissue origins | | |
| Macrophage_ | Macrophage cells with | P2RX6 | 0.0591444 |
| Other | other tissue origins | | |
| Macrophage_ | Macrophage cells with | PBXIP1 | −0.074238 |
| Other | other tissue origins | | |
| Macrophage_ | Macrophage cells with | PLN | −0.095633 |
| Other | other tissue origins | | |
| Macrophage_ | Macrophage cells with | PPP1R3C | −0.094268 |
| Other | other tissue origins | | |
| Macrophage_ | Macrophage cells with | PRKCE | −0.062716 |
| Other | other tissue origins | | |
| Macrophage_ | Macrophage cells with | SEC14L4 | −0.06227 |
| Other | other tissue origins | | |
| Macrophage_ | Macrophage cells with | SLC13A3 | 0.0941934 |
| Other | other tissue origins | | |
| Macrophage_ | Macrophage cells with | SMOC2 | 0.0588569 |
| Other | other tissue origins | | |
| Macrophage_ | Macrophage cells with | TAF11 | 0.045049 |
| Other | other tissue origins | | |
| Macrophage_ | Macrophage cells with | TBX4 | −0.520128 |
| Other | other tissue origins | | |
| Macrophage_ | Macrophage cells with | TEAD2 | −0.30886 |
| Other | other tissue origins | | |
| Macrophage_ | Macrophage cells with | TMOD1 | 0.1696014 |
| Other | other tissue origins | | |
| Macrophage_ | Macrophage cells with | TNNC1 | −0.162403 |
| Other | other tissue origins | | |
| Macrophage_ | Macrophage cells with | TSPAN10 | 0.1474647 |
| Other | other tissue origins | | |
| Macrophage_ | Macrophage cells with | WDR36 | 0.0373679 |
| Other | other tissue origins | | |
| Macrophage_ | Macrophage cells with | XRCC1 | 0.2726984 |
| Other | other tissue origins | | |
| Macrophage_ | Macrophage cells with | (Intercept) | −7.886531 |
| Peritoneal_ | peritoneal cavity tissue | | |
| Cavity | origins | | |
| Macrophage_ | Macrophage cells with | BCAM | 0.0034573 |
| Peritoneal_ | peritoneal cavity tissue | | |
| Cavity | origins | | |
| Macrophage_ | Macrophage cells with | FABP7 | 0.0139429 |
| Peritoneal_ | peritoneal cavity tissue | | |
| Cavity | origins | | |
| Macrophage_ | Macrophage cells with | FAM20A | 0.1139873 |
| Peritoneal_ | peritoneal cavity tissue | | |
| Cavity | origins | | |
| Macrophage_ | Macrophage cells with | FAM78B | 0.2362067 |
| Peritoneal_ | peritoneal cavity tissue | | |
| Cavity | origins | | |
| Macrophage_ | Macrophage cells with | GLB1 | 0.0496994 |
| Peritoneal_ | peritoneal cavity tissue | | |
| Cavity | origins | | |
| Macrophage_ | Macrophage cells with | HAL | 0.1725152 |
| Peritoneal_ | peritoneal cavity tissue | | |
| Cavity | origins | | |
| Macrophage_ | Macrophage cells with | KDM6B | −0.022042 |
| Peritoneal_ | peritoneal cavity tissue | | |
| Cavity | origins | | |
| Macrophage_ | Macrophage cells with | LIPN | 0.0626554 |
| Peritoneal_ | peritoneal cavity tissue | | |
| Cavity | origins | | |
| Macrophage_ | Macrophage cells with | MST1R | 0.1016218 |
| Peritoneal_ | peritoneal cavity tissue | | |
| Cavity | origins | | |
| Macrophage_ | Macrophage cells with | PADI4 | 0.0385276 |
| Peritoneal_ | peritoneal cavity tissue | | |
| Cavity | origins | | |
| Macrophage_ | Macrophage cells with | PYCARD | 0.2990131 |
| Peritoneal_ | peritoneal cavity tissue | | |
| Cavity | origins | | |
| Macrophage_ | Macrophage cells with | RARB | 0.5058355 |
| Peritoneal_ | peritoneal cavity tissue | | |
| Cavity | origins | | |
| Macrophage_ | Macrophage cells with | RNF141 | 0.0399126 |
| Peritoneal_ | peritoneal cavity tissue | | |
| Cavity | origins | | |
| Macrophage_ | Macrophage cells with | SPATA13 | −0.18623 |
| Peritoneal_ | peritoneal cavity tissue | | |
| Cavity | origins | | |
| Macrophage_ | Macrophage cells with | VAMP4 | 0.1420015 |
| Peritoneal_ | peritoneal cavity tissue | | |
| Cavity | origins | | |
| Macrophage_ | Macrophage cells with | VSIG8 | 0.1705707 |
| Peritoneal_ | peritoneal cavity tissue | | |
| Cavity | origins | | |
| Monocyte_ | Monocyte cells | (Intercept) | −3.1518 |
| all | | | |
| Monocyte_ | Monocyte cells | ACE | 0.187378 |
| all | | | |
| Monocyte_ | Monocyte cells | ALDH3B2 | −0.040236 |
| all | | | |
| Monocyte_ | Monocyte cells | ARHGEF37 | 0.2068529 |
| all | | | |
| Monocyte_ | Monocyte cells | CCNO | −0.082706 |
| all | | | |
| Monocyte_ | Monocyte cells | CENPE | −0.118123 |
| all | | | |
| Monocyte_ | Monocyte cells | CEP89 | −0.026038 |
| all | | | |
| Monocyte_ | Monocyte cells | CYP4B1 | 0.0017585 |
| all | | | |
| Monocyte_ | Monocyte cells | DAXX | 0.2480184 |
| all | | | |
| Monocyte_ | Monocyte cells | DCBLD2 | −0.059914 |
| all | | | |
| Monocyte_ | Monocyte cells | GABBR1 | −0.291848 |
| all | | | |
| Monocyte_ | Monocyte cells | GPLD1 | 0.1297842 |
| all | | | |
| Monocyte_ | Monocyte cells | JADE1 | −0.277935 |
| all | | | |
| Monocyte_ | Monocyte cells | KIF18A | −0.06814 |
| all | | | |
| Monocyte_ | Monocyte cells | LETM2 | −0.123993 |
| all | | | |
| Monocyte_ | Monocyte cells | MARVELD1 | −0.065281 |
| all | | | |
| Monocyte_ | Monocyte cells | MCF2L | 0.1285281 |
| all | | | |
| Monocyte_ | Monocyte cells | MSRB2 | −0.048449 |
| all | | | |
| Monocyte_ | Monocyte cells | NECAP1 | 0.0826144 |
| all | | | |
| Monocyte_ | Monocyte cells | OTUD7B | −0.071734 |
| all | | | |
| Monocyte_ | Monocyte cells | PLAC8 | 0.0420314 |
| all | | | |
| Monocyte_ | Monocyte cells | PLEKHF2 | 0.0183138 |
| all | | | |
| Monocyte_ | Monocyte cells | PPP1R3G | −0.112416 |
| all | | | |
| Monocyte_ | Monocyte cells | RSPO1 | 0.3097449 |
| all | | | |
| Monocyte_ | Monocyte cells | SFTPB | 0.1710195 |
| all | | | |
| Monocyte_ | Monocyte cells | SH2D6 | 0.2448665 |
| all | | | |
| Monocyte_ | Monocyte cells | SYNE2 | −0.018358 |
| all | | | |
| Monocyte_ | Monocyte cells | SYT8 | −0.09532 |
| all | | | |
| Monocyte_ | Monocyte cells | TBC1D19 | −0.06701 |
| all | | | |
| Monocyte_ | Monocyte cells | TONSL | −0.006234 |
| all | | | |
| Monocyte_ | Monocyte cells | VSIG8 | −0.079298 |
| all | | | |
| Monocyte_ | Monocyte cells | ZFYVE9 | 0.143403 |
| all | | | |
| Monocyte_ | Monocyte cells | ZNF76 | −0.137201 |
| all | | | |
| B_all | B cells | (Intercept) | −1.327731 |
| B_all | B cells | BFSP2 | 0.1144329 |
| B_all | B cells | FYB1 | −0.043922 |
| B_all | B cells | IGHV1-24 | 0.6209573 |
| B_all | B cells | IGHV1-46 | 9.94E−15 |
| B_all | B cells | IGHV1-69 | 4.58E−16 |
| B_all | B cells | IGHV1 | 0.1739779 |
| | | OR15-9 | |
| B_all | B cells | IGHV2-26 | 0.0594361 |
| B_all | B cells | IGHV2-5 | 3.64E−15 |
| B_all | B cells | IGHV2-70 | 9.80E−17 |
| B_all | B cells | IGHV2-70D | 3.92E−17 |
| B_all | B cells | IGHV3-15 | 0.0022022 |
| B_all | B cells | KCNG1 | 0.0696491 |
| B_all | B cells | MZB1 | 0.0639528 |
| B_all | B cells | PAX5 | 0.0165548 |
| B_all | B cells | PPM1H | −0.487917 |
| B_all | B cells | PPPIR36 | 0.0671497 |
| B_all | B cells | VPREB3 | 0.270114 |
| Macrophage_ | Macrophage cells | (Intercept) | 0.7265421 |
| all | | | |
| Macrophage_ | Macrophage cells | ACY3 | 0.0151788 |
| all | | | |
| Macrophage_ | Macrophage cells | AGMO | 0.0121083 |
| all | | | |
| Macrophage_ | Macrophage cells | ANKRD12 | −0.186162 |
| all | | | |
| Macrophage_ | Macrophage cells | ASB4 | 0.1293867 |
| all | | | |
| Macrophage_ | Macrophage cells | ATP13A5 | −0.008178 |
| all | | | |
| Macrophage_ | Macrophage cells | B3GLCT | 0.016268 |
| all | | | |
| Macrophage_ | Macrophage cells | C2CD3 | −0.003533 |
| all | | | |
| Macrophage_ | Macrophage cells | CANX | 0.040146 |
| all | | | |
| Macrophage_ | Macrophage cells | CASC3 | −0.007099 |
| all | | | |
| Macrophage_ | Macrophage cells | CCL24 | 0.0082989 |
| all | | | |
| Macrophage_ | Macrophage cells | CD2BP2 | −0.150476 |
| all | | | |
| Macrophage_ | Macrophage cells | CPE | −0.004293 |
| all | | | |
| Macrophage_ | Macrophage cells | CXCL14 | 0.0657919 |
| all | | | |
| Macrophage_ | Macrophage cells | DENND3 | −0.008955 |
| all | | | |
| Macrophage_ | Macrophage cells | EFEMP2 | 0.1616696 |
| all | | | |
| Macrophage_ | Macrophage cells | ELF4 | −0.008196 |
| all | | | |
| Macrophage_ | Macrophage cells | EME2 | −0.161664 |
| all | | | |
| Macrophage_ | Macrophage cells | GBGT1 | 0.2021341 |
| all | | | |
| Macrophage_ | Macrophage cells | GPATCH11 | −0.050541 |
| all | | | |
| Macrophage_ | Macrophage cells | GPLD1 | −0.016197 |
| all | | | |
| Macrophage_ | Macrophage cells | HSH2D | −0.036164 |
| all | | | |
| Macrophage_ | Macrophage cells | IL1A | 0.0584859 |
| all | | | |
| Macrophage_ | Macrophage cells | MGMT | 0.1032007 |
| all | | | |
| Macrophage_ | Macrophage cells | MRC1 | 0.1710779 |
| all | | | |
| Macrophage_ | Macrophage cells | MYO1H | −0.071332 |
| all | | | |
| Macrophage_ | Macrophage cells | MYO7A | 0.0757502 |
| all | | | |
| Macrophage_ | Macrophage cells | NEURL1 | 0.0897319 |
| all | | | |
| Macrophage_ | Macrophage cells | NXPE3 | −0.051996 |
| all | | | |
| Macrophage_ | Macrophage cells | PCP4L1 | −0.046003 |
| all | | | |
| Macrophage_ | Macrophage cells | PKP3 | −0.003309 |
| all | | | |
| Macrophage_ | Macrophage cells | PLAC8 | −0.040104 |
| all | | | |
| Macrophage_ | Macrophage cells | PPP1R3G | 0.1983249 |
| all | | | |
| Macrophage_ | Macrophage cells | RHOC | 0.057847 |
| all | | | |
| Macrophage_ | Macrophage cells | RSPO1 | −0.064499 |
| all | | | |
| Macrophage_ | Macrophage cells | SERPINB6 | 0.1218214 |
| all | | | |
| Macrophage_ | Macrophage cells | SH2D3C | −0.16929 |
| all | | | |
| Macrophage_ | Macrophage cells | SHLD2 | −0.141058 |
| all | | | |
| Macrophage_ | Macrophage cells | SLCO1C1 | −0.095745 |
| all | | | |
| Macrophage_ | Macrophage cells | SMARCD3 | −0.00475 |
| all | | | |
| Macrophage_ | Macrophage cells | SNRPC | −0.03814 |
| all | | | |
| Macrophage_ | Macrophage cells | SPOCK2 | −0.036981 |
| all | | | |
| Macrophage_ | Macrophage cells | TRIM56 | −0.063329 |
| all | | | |
| Macrophage_ | Macrophage cells | VRK3 | −0.02568 |
| all | | | |
| Macrophage_ | Macrophage cells | VTN | −0.090261 |
| all | | | |
| Macrophage_ | Macrophage cells | VWA3B | −0.018121 |
| all | | | |
| Macrophage_ | Macrophage cells | ZNF217 | −0.035176 |
| all | | | |

Each tumor's gene expression data was processed identically. Experiment data was converted to a DGE list using the R (version 4.1.1) function SE2DGEList in the edgeR package (Robinson, McCarthy et al. 2010). Duplicate genes were reduced to a single gene by selecting the gene with the maximum standard deviation across all samples in the data set. Mouse gene names were converted to human orthologs using biomaRt (Durinck, Spellman et al. 2009). When there was gene duplication in orthologs, the gene with greatest homology with a human ortholog was retained. HUGO Gene Nomenclature Committee (HGNC) names for each gene were added using the biomarRt package. Genes without a HGNC designation were removed from the analysis. Using TMM normalization in the EdgeR package, normalization factors were calculated, applied, and log 2 transformed counts per million (cpm) were determined for each gene for each sample. The distribution of cpm and the standard deviation of genes across samples was plotted, and cutoffs determined to remove very low expressing genes. Samples were then renormalized using TMM after these genes were removed.

### Creation of Working Set of Immune Markers.

Genes that are differentially expressed in specific immune cell types were nominated from a variety of published sources. To validate the given association of the genes with immune cell types, RNASeq data from defined mouse immune cell populations from the ImmGen project GSE109125 (Yoshida, Lareau et al. 2019) and GSE122108 (ImmGen 2016) and defined human immune cell populations GSE22886 (Abbas, Baldwin et al. 2005) were used. As definitions of immune cell type differed between studies, the cell types were simplified to B lymphoid, non-B lymphoid, and myeloid types. A Student T-test was used to determine if the expression in the corresponding category of defined cells was greater than expected by chance. Genes with a positive T score and a p value less than 0.05 in either the mouse or human RNASeq data sets were retained for further use, resulting in 680 immune markers. To keep the number of markers for each cell type balanced as some cell types had a markedly greater number of nominated markers than others, the genes were ranked by p value and a maximum of 24 markers (mean of number of genes associated with each specific cell type, e.g. ‘M2D macrophage’) were retained. If the number of genes within a particular annotation was less than 12 (1st quartile of genes associated with each cell type), then no attempt was made to model that cell type. Published sources for certain genes used for model building in the present disclosure are presented in Table 41B.

TABLE 41B
Published sources from which candidate immune genes were used for model building,

| Source | Link or publication information |
|---|---|
| CellMarker | Congxue Hu, Tengyue Li, Yingqi Xu, Xinxin Zhang, Feng Li, Jing Bai, |
| | Jing Chen, Wenqi Jiang, Kaiyue Yang, Qi Ou, Xia Li, Peng Wang, |
| | Yunpeng Zhang, CellMarker 2.0: an updated database of manually |
| | curated cell markers in human/mouse and web tools based on scRNA- |
| | seq data, \ddot{1}¿½Nucleic Acids Research, Volume 51, Issue D1, 6 Jan. |
| | 2023, Pages D870\ddot{1}¿½D876, \ddot{1}¿½ |
| | https://protect- |
| | us.mimecast.com/s/g1s7CPN6MGtZgvApC6p2C_?domain=doi.org |
| Pangluo DB | Oscar Franz\ddot{1}¿½n, Li-Ming Gan, Johan L M |
| | Bj\ddot{1}¿½rkegren, \ddot{1}¿½PanglaoDB: a web server for exploration of mouse |
| | and human single-cell RNA sequencing data, \ddot{1}¿½Database, Volume |
| | 2019, 2019, baz046, \ddot{1}¿½doi: 10.1093/database/baz046 |
| BIORAD | https://protect- |
| | us.mimecast.com/s/xu_iCQWANXC9VB40UA9brP?domain=bio-rad- |
| | antibodies.com |
| Macrophage Polarization - Mini-review | bio-rad-antibodies.com |
| \| Bio-Rad (https://protect- | |
| us.mimecast.com/s/KOY4CR68MXCQ | |
| YnP2fQhJ0T?domain=bio-rad- | |
| antibodies.com) | |
| Wang L X, Zhang S X, Wu H J, Rong | Wang L X, Zhang S X, Wu H J, Rong X L, Guo J. M2b macrophage |
| XL, Guo J. M2b macrophage | polarization and its roles in diseases. J Leukoc Biol. 2019; 106(2): 345- |
| polarization and its roles in diseases. J | 358. |
| Leukoc Biol. 2019; 106(2): 345-358. | |
| Jablonski K A, Amici S A, Webb L M, et | Jablonski K A, Amici S A, Webb L M, et al. Novel Markers to Delineate |
| al. Novel Markers to Delineate Murine | Murine M1 and M2 Macrophages. PLoS One. 2015; 10(12) |
| M1 and M2 Macrophages. PLoS One. | |
| 2015; 10(12) | |
| Trombetta A C, Soldano S, Contini P, et | Trombetta A C, Soldano S, Contini P, et al. A circulating cell |
| al. A circulating cell population | population showing both M1 and M2 monocyte/macrophage surface |
| showing both M1 and M2 | markers characterizes systemic sclerosis patients with lung |
| monocyte/macrophage surface markers | involvement. Respir Res. 2018; 19(1): 186. |
| characterizes systemic sclerosis patients | |
| with lung involvement. Respir Res. | |
| 2018; 19(1): 186. | |
| Jablonski K A, Amici S A, Webb L M, et | Jablonski K A, Amici S A, Webb L M, et al. Novel Markers to Delineate |
| al. Novel Markers to Delineate Murine | Murine M1 and M2 Macrophages. PLoS One. 2015; 10(12) |
| M1 and M2 Macrophages. PLoS One. | |
| 2015; 10(12) | |
| Jablonski K A, Amici S A, Webb L M, et | Jablonski K A, Amici S A, Webb L M, et al. Novel Markers to Delineate |
| al. Novel Markers to Delineate Murine | Murine M1 and M2 Macrophages. PLoS One. 2015; 10(12) |
| M1 and M2 Macrophages. PLoS One. | |
| 2015; 10(12) | |
| Wang L X, Zhang S X, Wu H J, Rong | Wang L X, Zhang S X, Wu H J, Rong X L, Guo J. M2b macrophage |
| X L, Guo J. M2b macrophage | polarization and its roles in diseases. J Leukoc Biol. 2019; 106(2): 345- |
| polarization and its roles in diseases. J | 358. |
| Leukoc Biol. 2019; 106(2): 345-358. | |
| J Immunol. 2016 Mar. 15; 196(6): | Poczobutt J M, De S, Yadav V K, Nguyen T T, Li H, Sippel T R, Weiser- |
| 2847\ddot{1}¿½2859. | Evans M C, Nemenoff R A. Expression Profiling of Macrophages |
| | Reveals Multiple Populations with Distinct Biological Roles in an |
| | Immunocompetent Orthotopic Model of Lung Cancer. J Immunol. |
| | 2016 Mar. 15; 196(6): 2847-59. doi: 10.4049/jimmunol.1502364. Epub |
| | 2016 Feb. 12. PMID: 26873985; PMCID: PMC4779748. |
| Macrophage Polarization - Mini-review | Macrophage Polarization - Mini-review \| Bio-Rad (https://protect- |
| \| Bio-Rad (https://protect- | us.mimecast.com/s/KOY4CR68MXCQYnP2fQhJ0T?domain=bio-rad- |
| us.mimecast.com/s/KOY4CR68MXCQ | antibodies.com) |
| YnP2fQhJ0T?domain=bio-rad- | |
| antibodies.com) | |
| Tumor Associated Neutrophils. Their | Masucci M T, Minopoli M and Carriero M V (2019) Tumor Associated |
| Role in Tumorigenesis, Metastasis, | Neutrophils. Their Role in Tumorigenesis, Metastasis, Prognosis |
| Prognosis and Therapy (https://protect- | and Therapy. Front. Oncol. 9: 1146. doi: 10.3389/fonc.2019.01146 |
| us.mimecast.com/s/IfjiCVOJ5WiXB0jG | |
| SEuhiF?domain=nih.gov) | |
| Wang X, Qiu L, Li Z, Wang X Y, Yi H. | Wang X, Qiu L, Li Z, Wang X Y, Yi H. Understanding the Multifaceted |
| Understanding the Multifaceted Role of | Role of Neutrophils in Cancer and Autoimmune Diseases. Front |
| Neutrophils in Cancer and Autoimmune | Immunol. 2018; 9: 2456. |
| Diseases. Front Immunol. 2018; 9: 2456. | |
| Morgan A. Giese, Laurel E. Hind, Anna | Morgan A. Giese, Laurel E. Hind, Anna Huttenlocher; Neutrophil |
| Huttenlocher; Neutrophil plasticity in | plasticity in the tumor microenvironment. Blood 2019; 133 (20): |
| the tumor microenvironment. Blood | 2159\ddot{1}¿½2167. |
| 2019; 133 (20): 2159\ddot{1}¿½2167. | |
| BioCompare | https://protect- |
| | us.mimecast.com/s/wb7KCW6Ww1Cx3zwJIOCL_r?domain=biocompare.com |

### Creation of Network Modeling of Shared Immune Genes

Gene expression data for the training sets of solid tumors in the TCGA20 (lung squamous cell, lung adenocarcinoma, breast, ovarian, kidney clear cell, head and neck, prostate, colon, bladder, pancreas, kidney papillary, rectal, kidney chromophobe, esophageal, stomach cancers) were each individually scaled. The RNASeq expression data sets were limited to the immune markers as described (N=758). These scaled data sets were then fuzzy clustered using fclust (Ferraro, Giordani et al. 2019), with k=15. Clusters of genes identified by this process were limited to genes whose expression was three standard deviations above the mean for all genes. Clusters were only selected for further use if they contained at least five genes. These selected clusters for all fifteen tumors were combined and then used to create a network, and clusters of genes were defined by the cluster_louvain function in igraph (Blondel, Guillaume et al. 2008). Each set of genes defined in these network models were named based on annotation of the contributing genes in terms of their curated association with immune cell types Weights for each gene in a network signature were derived using the mean of all fuzzy clustering gene scores. Samples are scored with these models via a determining of the weighted mean score for each sample using the genes of each network signature set. Genes and coefficients are presented in Table 42.

TABLE 42
Immune gene network signatures.

| | Simplified | | |
|---|---|---|---|
| Model name | model name | Gene name | Gene weight |
| NC_Tc_PC_Tr_B | non-B | PTPRC | 0.917325418 |
| | Lymphoid 1 | | |
| NC_Tc_PC_Tr_B | non-B | IKZF1 | 0.854261396 |
| | Lymphoid 1 | | |
| NC_Tc_PC_Tr_B | non-B | IRF8 | 0.83718805 |
| | Lymphoid 1 | | |
| NC_Tc_PC_Tr_B | non-B | CCR2 | 0.626339138 |
| | Lymphoid 1 | | |
| NC_Tc_PC_Tr_B | non-B | CD40LG | 0.700167609 |
| | Lymphoid 1 | | |
| NC_Tc_PC_Tr_B | non-B | CSF2RB | 0.653807747 |
| | Lymphoid 1 | | |
| NC_Tc_PC_Tr_B | non-B | CCR4 | 0.691980433 |
| | Lymphoid 1 | | |
| NC_Tc_PC_Tr_B | non-B | PTGDS | 0.577573099 |
| | Lymphoid 1 | | |
| NC_Tc_PC_Tr_B | non-B | CD37 | 0.804972636 |
| | Lymphoid 1 | | |
| NC_Tc_PC_Tr_B | non-B | IL16 | 0.780911078 |
| | Lymphoid 1 | | |
| NC_Tc_PC_Tr_B | non-B | CLEC10A | 0.579434053 |
| | Lymphoid 1 | | |
| NC_Tc_PC_Tr_B | non-B | IL7R | 0.691393804 |
| | Lymphoid 1 | | |
| NC_Tc_PC_Tr_B | non-B | CD69 | 0.50293393 |
| | Lymphoid 1 | | |
| NC_Tc_PC_Tr_B | non-B | SELL | 0.600787357 |
| | Lymphoid 1 | | |
| NC_Tc_PC_Tr_B | non-B | CD28 | 0.686122434 |
| | Lymphoid 1 | | |
| NC_Tc_PC_Tr_B | non-B | ICOS | 0.664203652 |
| | Lymphoid 1 | | |
| NC_Tc_PC_Tr_B | non-B | PTPN22 | 0.61607136 |
| | Lymphoid 1 | | |
| NC_Tc_PC_Tr_B | non-B | BCL11B | 0.609119483 |
| | Lymphoid 1 | | |
| NC_Tc_PC_Tr_B | non-B | RGS18 | 0.370643898 |
| | Lymphoid 1 | | |
| NC_Tc_PC_Tr_B | non-B | FCGR2C | 0.256895259 |
| | Lymphoid 1 | | |
| NC_Tc_PC_Tr_B | non-B | TMEM156 | 0.270859513 |
| | Lymphoid 1 | | |
| B_Bmem | B cells | BLK | 0.890181803 |
| B_Bmem | B cells | TNFRSF13B | 0.876186917 |
| B_Bmem | B cells | CD19 | 0.910624235 |
| B_Bmem | B cells | CD79B | 0.897000018 |
| B_Bmem | B cells | POU2AF1 | 0.920626349 |
| B_Bmem | B cells | IGLC3 | 0.952422944 |
| B_Bmem | B cells | IGHA1 | 0.969434783 |
| B_Bmem | B cells | CD79A | 0.988008884 |
| B_Bmem | B cells | IGLC2 | 0.987185163 |
| B_Bmem | B cells | IGKC | 0.994055721 |
| B_Bmem | B cells | MZB1 | 0.990953944 |
| B_Bmem | B cells | PNOC | 0.911511441 |
| B_Bmem | B cells | CD27 | 0.915284037 |
| B_Bmem | B cells | FCRL2 | 0.848730854 |
| B_Bmem | B cells | IGHM | 0.940097522 |
| B_Bmem | B cells | JCHAIN | 0.952169016 |
| B_Bmem | B cells | TNFRSF13C | 0.820399226 |
| B_Bmem | B cells | VPREB3 | 0.870654492 |
| B_Bmem | B cells | CR2 | 0.836133953 |
| B_Bmem | B cells | LY9 | 0.86687041 |
| B_Bmem | B cells | CD22 | 0.943096213 |
| Tgd_T_Tm | non-B | IL2RG | 0.849230312 |
| | Lymphoid 3 | | |
| Tgd_T_Tm | non-B | CD3G | 0.881440415 |
| | Lymphoid 3 | | |
| Tgd_T_Tm | non-B | CXCR3 | 0.874962552 |
| | Lymphoid 3 | | |
| Tgd_T_Tm | non-B | SH2D1A | 0.894351476 |
| | Lymphoid 3 | | |
| Tgd_T_Tm | non-B | CD2 | 0.975933021 |
| | Lymphoid 3 | | |
| Tgd_T_Tm | non-B | CD3E | 0.984355314 |
| | Lymphoid 3 | | |
| Tgd_T_Tm | non-B | CD3D | 0.895022156 |
| | Lymphoid 3 | | |
| Tgd_T_Tm | non-B | TRBC2 | 0.969101205 |
| | Lymphoid 3 | | |
| Tgd_T_Tm | non-B | ITK | 0.884268188 |
| | Lymphoid 3 | | |
| Tgd_T_Tm | non-B | SEPTIN1 | 0.705468055 |
| | Lymphoid 3 | | |
| Tgd_T_Tm | non-B | TBC1D10C | 0.773429372 |
| | Lymphoid 3 | | |
| Tgd_T_Tm | non-B | LCK | 0.918259358 |
| | Lymphoid 3 | | |
| Tgd_T_Tm | non-B | PDCD1 | 0.757961539 |
| | Lymphoid 3 | | |
| Tgd_T_Tm | non-B | CTLA4 | 0.738854239 |
| | Lymphoid 3 | | |
| Tgd_T_Tm | non-B | TIGIT | 0.889416557 |
| | Lymphoid 3 | | |
| Tgd_T_Tm | non-B | CCL5 | 0.902009794 |
| | Lymphoid 3 | | |
| Tgd_T_Tm | non-B | TBX21 | 0.783005531 |
| | Lymphoid 3 | | |
| Tgd_T_Tm | non-B | CD7 | 0.806232456 |
| | Lymphoid 3 | | |
| Tgd_T_Tm | non-B | CCL4 | 0.848241653 |
| | Lymphoid 3 | | |
| Tgd_T_Tm | non-B | TRDC | 0.670793137 |
| | Lymphoid 3 | | |
| Tgd_T_Tm | non-B | FASLG | 0.948719515 |
| | Lymphoid 3 | | |
| D_N_M | Myeloid 1 | CD180 | 0.818123022 |
| D_N_M | Myeloid 1 | CD14 | 0.862053164 |
| D_N_M | Myeloid 1 | FCGR1A | 0.788506002 |
| D_N_M | Myeloid 1 | AIF1 | 0.878369526 |
| D_N_M | Myeloid 1 | TLR8 | 0.855379385 |
| D_N_M | Myeloid 1 | FCGR3A | 0.903577469 |
| D_N_M | Myeloid 1 | HAVCR2 | 0.95209179 |
| D_N_M | Myeloid 1 | MNDA | 0.877753077 |
| D_N_M | Myeloid 1 | CMKLR1 | 0.856603997 |
| D_N_M | Myeloid 1 | CSF1R | 0.888130199 |
| D_N_M | Myeloid 1 | SIGLEC1 | 0.85613325 |
| D_N_M | Myeloid 1 | C3AR1 | 0.948899775 |
| D_N_M | Myeloid 1 | CCR1 | 0.860443369 |
| D_N_M | Myeloid 1 | MSR1 | 0.885921785 |
| D_N_M | Myeloid 1 | TYROBP | 0.896458165 |
| D_N_M | Myeloid 1 | FCGR2A | 0.82660907 |
| D_N_M | Myeloid 1 | CD163 | 0.908990209 |
| D_N_M | Myeloid 1 | MS4A4A | 0.954017257 |
| D_N_M | Myeloid 1 | VSIG4 | 0.864592262 |
| D_N_M | Myeloid 1 | OLR1 | 0.641709136 |
| D_N_M | Myeloid 1 | CD86 | 0.942643847 |
| N2_M_M1_N | Myeloid 4 | CSF3R | 0.715215998 |
| N2_M_M1_N | Myeloid 4 | CXCL2 | 0.595410915 |
| N2_M_M1_N | Myeloid 4 | SLC11A1 | 0.689708163 |
| N2_M_M1_N | Myeloid 4 | MCEMP1 | 0.768803468 |
| N2_M_M1_N | Myeloid 4 | TREM1 | 0.824650671 |
| N2_M_M1_N | Myeloid 4 | AQP9 | 0.811375587 |
| N2_M_M1_N | Myeloid 4 | FPR1 | 0.719975361 |
| N2_M_M1_N | Myeloid 4 | FPR2 | 0.737412524 |
| N2_M_M1_N | Myeloid 4 | C15orf48 | 0.570093343 |
| N2_M_M1_N | Myeloid 4 | IL1A | 0.820259972 |
| N2_M_M1_N | Myeloid 4 | IL1B | 0.695235604 |
| N2_M_M1_N | Myeloid 4 | CXCL1 | 0.689400321 |
| N2_M_M1_N | Myeloid 4 | CXCL8 | 0.75884384 |
| N2_M_M1_N | Myeloid 4 | S100A8 | 0.826027987 |
| N2_M_M1_N | Myeloid 4 | S100A9 | 0.866857554 |
| N2_M_M1_N | Myeloid 4 | IL1RN | 0.735164439 |
| N2_M_M1_N | Myeloid 4 | LY6D | 0.826119538 |
| N2_M_M1_N | Myeloid 4 | CCL7 | 0.821428177 |
| N2_M_M1_N | Myeloid 4 | OSM | 0.782890491 |
| N2_M_M1_N | Myeloid 4 | TNF | 0.420337801 |
| N2_M_M1_N | Myeloid 4 | CSF2 | 0.593191201 |
| Mo_Baso_M_M2_ | Myeloid 3 | GIMAP4 | 0.83438145 |
| Mast_MK | | | |
| Mo_Baso_M_M2_ | Myeloid 3 | GIMAP5 | 0.829366114 |
| Mast_MK | | | |
| Mo_Baso_M_M2_ | Myeloid 3 | GIMAP1 | 0.782323816 |
| Mast_MK | | | |
| Mo_Baso_M_M2_ | Myeloid 3 | PLAU | 0.572969998 |
| Mast_MK | | | |
| Mo_Baso_M_M2_ | Myeloid 3 | HGF | 0.595192943 |
| Mast_MK | | | |
| Mo_Baso_M_M2 | Myeloid 3 | DYSF | 0.613825168 |
| Mast_MK | | | |
| Mo_Baso_M_M2_ | Myeloid 3 | ACE | 0.437208174 |
| Mast_MK | | | |
| Mo_Baso_M_M2 | Myeloid 3 | S1PR1 | 0.859626705 |
| Mast_MK | | | |
| Mo_Baso_M_M2 | Myeloid 3 | VWF | 0.669753942 |
| Mast_MK | | | |
| Mo_Baso_M_M2_ | Myeloid 3 | ZBTB16 | 0.651179019 |
| Mast_MK | | | |
| Mo_Baso_M_M2_ | Myeloid 3 | CFP | 0.44063352 |
| Mast_MK | | | |
| Mo_Baso_M_M2_ | Myeloid 3 | FGFBP2 | 0.638039851 |
| Mast_MK | | | |
| Mo_Baso_M_M2_ | Myeloid 3 | CD36 | 0.717409509 |
| Mast_MK | | | |
| Mo_Baso_M_M2_ | Myeloid 3 | IGF1 | 0.691052422 |
| Mast_MK | | | |
| Mo_Baso_M_M2_ | Myeloid 3 | DAB2 | 0.557080727 |
| Mast_MK | | | |
| Mo_Baso_M_M2_ | Myeloid 3 | TLR4 | 0.626384948 |
| Mast_MK | | | |
| Mo_Baso_M_M2_ | Myeloid 3 | LRP1 | 0.604761842 |
| Mast_MK | | | |
| Mo_Baso_M_M2_ | Myeloid 3 | VCAM1 | 0.595818898 |
| Mast_MK | | | |
| Mo_Baso_M_M2_ | Myeloid 3 | FN1 | 0.621558355 |
| Mast_MK | | | |
| Mo_Baso_M_M2_ | Myeloid 3 | NT5E | 0.512603299 |
| Mast_MK | | | |
| Mo_Baso_M_M2_ | Myeloid 3 | TNFSF4 | 0.648997326 |
| Mast_MK | | | |
| Mast_D_Baso_Bn_ | Myeloid 2 | IL4R | 0.566193934 |
| MK_T_Th_Bmem | | | |
| Mast_D_Baso_Bn_ | Myeloid 2 | MAFG | 0.560799162 |
| MK_T_Th_Bmem | | | |
| Mast_D_Baso_Bn_ | Myeloid 2 | TRAF4 | 0.562977321 |
| MK_T_Th_Bmem | | | |
| Mast_D_Baso_Bn_ | Myeloid 2 | KIF5B | 0.679717963 |
| MK_T_Th_Bmem | | | |
| Mast_D_Baso_Bn_ | Myeloid 2 | STAT3 | 0.785791656 |
| MK_T_Th_Bmem | | | |
| Mast_D_Baso_Bn_ | Myeloid 2 | TET2 | 0.793797205 |
| MK_T_Th_Bmem | | | |
| Mast_D_Baso_Bn_ | Myeloid 2 | BCL7A | 0.650442077 |
| MK_T_Th_Bmem | | | |
| Mast_D_Baso_Bn_ | Myeloid 2 | SUPT3H | 0.475839276 |
| MK_T_Th_Bmem | | | |
| Mast_D_Baso_Bn_ | Myeloid 2 | EPOR | 0.716431271 |
| MK_T_Th_Bmem | | | |
| Mast_D_Baso_Bn_ | Myeloid 2 | PPARG | 0.620783906 |
| MK_T_Th_Bmem | | | |
| Mast_D_Baso_Bn_ | Myeloid 2 | ITGA2 | 0.692998153 |
| MK_T_Th_Bmem | | | |
| Mast_D_Baso_Bn_ | Myeloid 2 | MERTK | 0.571043006 |
| MK_T_Th_Bmem | | | |
| Mast_D_Baso_Bn_ | Myeloid 2 | LIMA1 | 0.709811988 |
| MK_T_Th_Bmem | | | |
| Mast_D_Baso_Bn_ | Myeloid 2 | SATB1 | 0.573754304 |
| MK_T_Th_Bmem | | | |
| Mast_D_Baso_Bn_ | Myeloid 2 | MITF | 0.682990431 |
| MK_T_Th_Bmem | | | |
| Mast_D_Baso_Bn_ | Myeloid 2 | IDS | 0.770547371 |
| MK_T_Th_Bmem | | | |
| Mast_D_Baso_Bn_ | Myeloid 2 | RALGPS2 | 0.790550186 |
| MK_T_Th_Bmem | | | |
| Mast_D_Baso_Bn_ | Myeloid 2 | BMPR2 | 0.81152926 |
| MK_T_Th_Bmem | | | |
| Mast_D_Baso_Bn_ | Myeloid 2 | ACSS2 | 0.660689281 |
| MK_T_Th_Bmem | | | |
| Mast_D_Baso_Bn_ | Myeloid 2 | HNMT | 0.588426363 |
| MK_T_Th_Bmem | | | |
| Mast_D_Baso_Bn_ | Myeloid 2 | HFE | 0.379911361 |
| MK_T_Th_Bmem | | | |
| Tgd_M2_N2_ | non-B | CD5L | 0.666840631 |
| Th17B_mem | Lymphoid 2 | | |
| Tgd_M2_N2_ | non-B | NDST2 | 0.616705557 |
| Th17B_mem | Lymphoid 2 | | |
| Tgd_M2_N2_ | non-B | PRTN3 | 0.613580976 |
| Th17B_mem | Lymphoid 2 | | |
| Tgd_M2_N2_ | non-B | TLR9 | 0.614786125 |
| Th17B_mem | Lymphoid 2 | | |
| Tgd_M2_N2_ | non-B | IL5 | 0.718193951 |
| Th17B_mem | Lymphoid 2 | | |
| Tgd_M2_N2_ | non-B | NMBR | 0.75771414 |
| Th17B_mem | Lymphoid 2 | | |
| Tgd_M2_N2_ | non-B | IZUMO1R | 0.665286008 |
| Th17B_mem | Lymphoid 2 | | |
| Tgd_M2_N2_ | non-B | IL2 | 0.557187991 |
| Th17B_mem | Lymphoid 2 | | |
| Tgd_M2_N2_ | non-B | CCL1 | 0.704954012 |
| Th17B_mem | Lymphoid 2 | | |
| Tgd_M2_N2_ | non-B | IL21 | 0.69890301 |
| Th17B_mem | Lymphoid 2 | | |
| Tgd_M2_N2_ | non-B | TRGV9 | 0.63922918 |
| Th17B_mem | Lymphoid 2 | | |
| Tgd_M2_N2_ | non-B | SPIC | 0.691740143 |
| Th17B_mem | Lymphoid 2 | | |
| Tgd_M2_N2_ | non-B | TRDV3 | 0.949214669 |
| Th17B_mem | Lymphoid 2 | | |
| Tgd_M2_N2_ | non-B | IL26 | 0.741259723 |
| Th17B_mem | Lymphoid 2 | | |
| Tgd_M2_N2_ | non-B | CLEC4C | 0.43513567 |
| Th17B_mem | Lymphoid 2 | | |
| Tgd_M2_N2_ | non-B | ARG1 | 0.537690306 |
| Th17B_mem | Lymphoid 2 | | |
| Tgd_M2_N2_ | non-B | ADGRG1 | 0.412855498 |
| Th17B_mem | Lymphoid 2 | | |

### Derivation of TIME Genes for Clustering

To build a gene set for examination of the TIME via clustering that encompasses the diverse genes that might play a role in the TIME in multiple tumor types, TCGA data for eighteen tumor types (lung squamous cell, lung adenocarcinoma, breast, ovarian, kidney clear cell, head and neck, prostate, melanoma, colon, bladder, pancreas, kidney papillary, sarcoma, rectal, B cell lymphoma, kidney chromophobe, esophageal and stomach cancers) were compared to the immune-modulatory, mesenchymal stem like, and mesenchymal TIME subtypes as defined by a 101 gene centroid previously described (Ring, Hout et al. 2016). In an examination of all possible sets of three distinct tumor types among the eighteen tumors, the union of the top 1,000 genes most highly correlated with each subtype were retained for each set of three tumors. These union gene data sets were sorted by the Euclidean distance of the IM, MSL and M clusters, and the top ranked gene sets that included all the eighteen tissues were retained. Gene sets that resulted in skewed cluster sizes which could generate high distances were identified as the standard deviation of the number of genes or tumors divided by the mean of the number of genes or tumors, and gene sets with a value less than 0.7 were not included, resulting in fifteen gene sets. In addition, a list of 27 genes previously described for the identification of the immuno-modulatory tumor environment (Nielsen, Ring et al. 2021) were added, resulting in a final unique gene list with N=2,967.

### Validation of ImmGen-Derived Models

The ImmGen derived models were validated using defined human immune cell populations GSE22886 (Abbas, Baldwin et al. 2005) and GSE74246 (Corces, Buenrostro et al. 2016). A Student T test was used to determine if the average score for a signature was greater than expected in either the signature's specific cell type (when data was available), or for the lineage of the signature (lymphoid or myeloid). All signatures not further refined by k-means subclasses or tissue of origin were significant when grouped by lineage (lymphoid or myeloid origins) in at least one data set. Those that were significant on both data sets were most of the non-refined signature sets: alpha-beta T cells, mature B cells, all B cells, dendritic cells, granulocytes, and macrophage cells. The CD8+ T cell and gamma-delta T cell models were not significant in both data sets.

### Description of Network Models of Co-Expressed Immune Markers in Relation to Other Immune Signatures

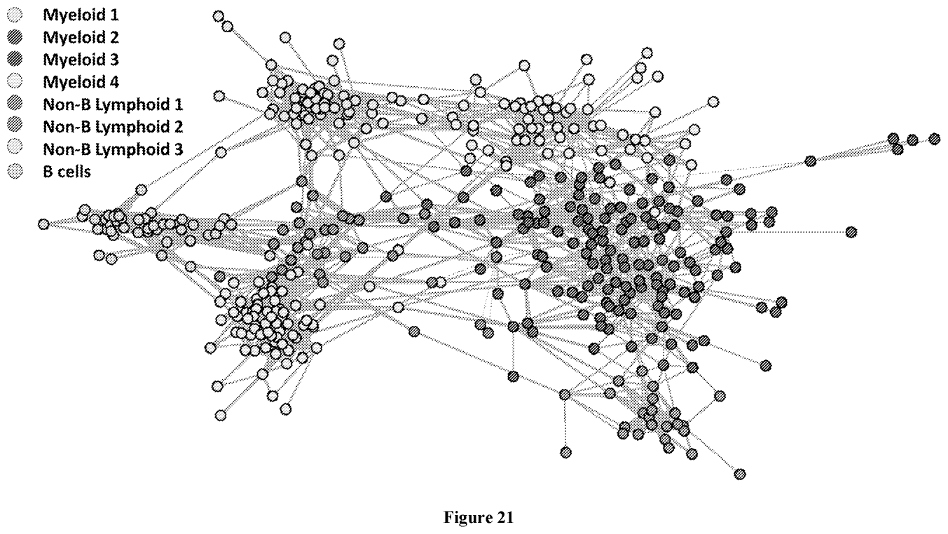

In addition to the ImmGen-derived signatures, signatures were also created to identify potential populations of immune cells that may co-infiltrate tumors. These signatures used genes previously declared to be markers for diverse immune cell types and were validated on mouse and human RNASeq data sets of defined cell populations Sets of co-expressed genes across fifteen different tumor types were identified, resulting in eight signatures (FIG. 21). These signatures all contained mixtures of immune markers, except for one which was comprised almost entirely of B cell markers (Table 43). However, the genes largely grouped into sets of genes attributed primarily to myeloid, non-B lymphoid, or B cell lineage. These lineage attributions were used to name the signatures, though it should be recognized that these signatures are associated with multiple immune cell types.

TABLE 43
Number of unique annotations for different
immune cell signatures in each lineage.

| | B cell | non-B cell | Myeloid |
|---|---|---|---|
| Signature | lineage | lymphoid lineage | lineage |
| B cells | 21 | 1 | 0 |
| non-B Lymphoid 1 | 6 | 12 | 6 |
| non-B Lymphoid 2 | 2 | 8 | 8 |
| non-B Lymphoid 3 | 1 | 20 | 1 |
| Myeloid 1 | 1 | 1 | 20 |
| Myeloid 2 | 3 | 3 | 16 |
| Myeloid 3 | 1 | 8 | 13 |
| Myeloid 4 | 1 | 1 | 19 |

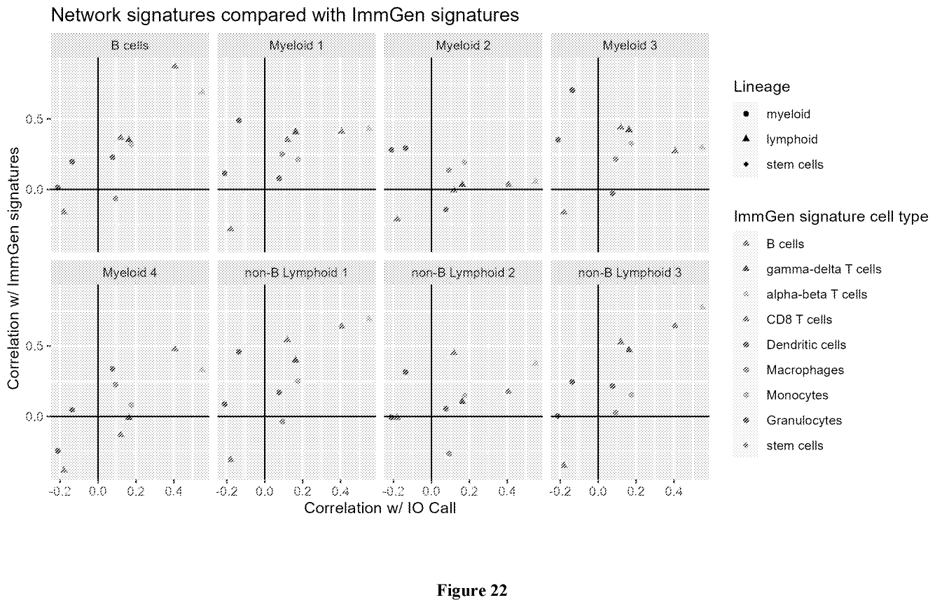

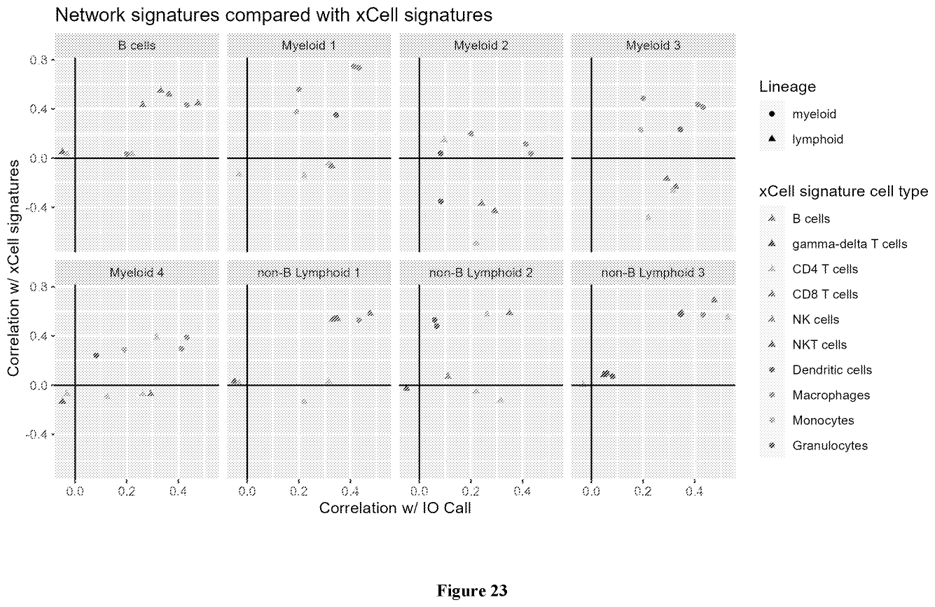

To further describe the network signatures, they were compared to the signatures derived from the ImmGen data sets across 7,162 samples comprised of 20 tumor types from The Cancer Genome Atlas (FIG. 22). Each network signature was compared to all the ImmGen-derived signatures and the correlation of each was contrasted with the correlation of each ImmGen signature to the 27 gene DTIO score previously described for the identification of the immuno-modulatory tumor environment (Nielsen, Ring et al. 2021). In a similar manner, the network signatures were compared to the xCell signatures (FIG. 23).

These comparisons demonstrate and validate that the novel network signatures described herein accurately identify and classify immune infiltrates. As seen in FIG. 23, the B cell signature is highly correlated with three xCell B cell signatures, which also are highly correlated with the DTIO score. Diversity between the signatures is shown in these comparisons. The myeloid signature 1 is correlated with three xCell macrophage signatures, as well as a dendritic and monocyte signature, while myeloid signature 4 is associated with two of those xCell macrophage signatures, as well as a granulocyte, monocyte and CD4+ T cell signature. Some of these associated signatures have low correlation with DTIO, suggesting that these network signatures are potentially identifying aspect of the infiltrate population not captured by DTIO. Similarly, the non-B lymphoid network signatures are correlated most strongly with xCell lymphoid signatures, though significant correlations with myeloid signatures are found in all these models. For example, the model non-B lymphoid 2 correlated with a CD8+ and CD4+ T cell signatures, but also with two granulocyte signatures, both of which have a low correlation with DTIO.

### Distribution of Network Signatures Between Tumor Tissue and TIME Subsets

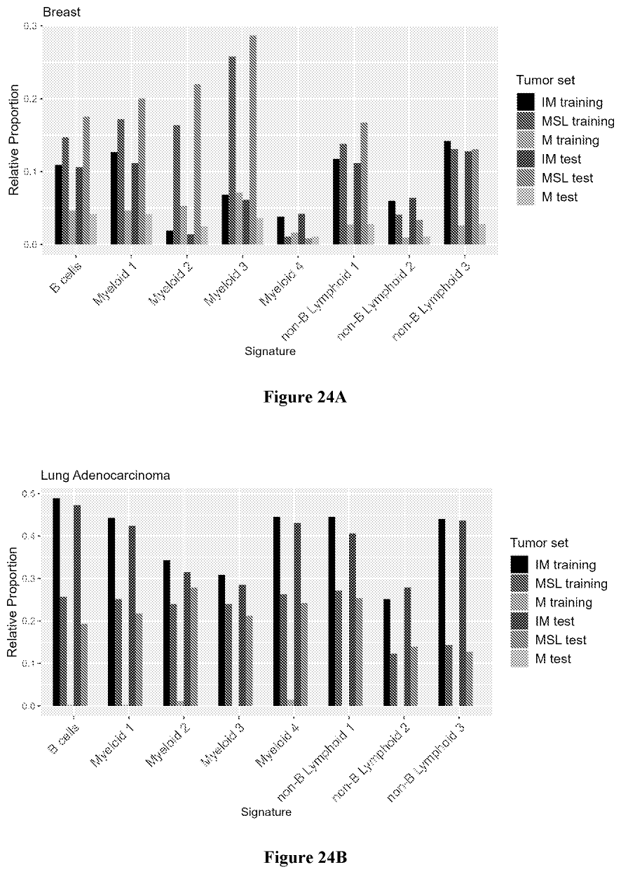

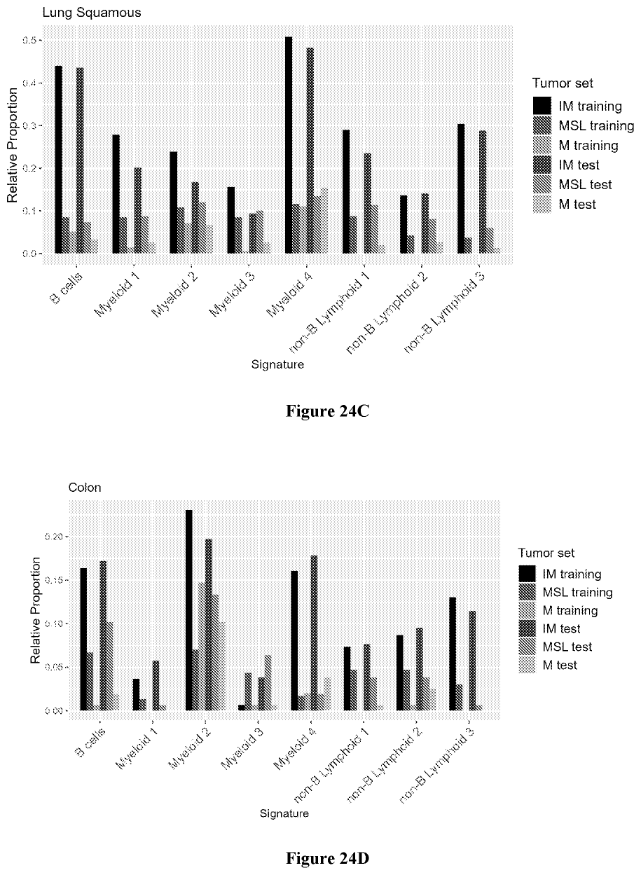

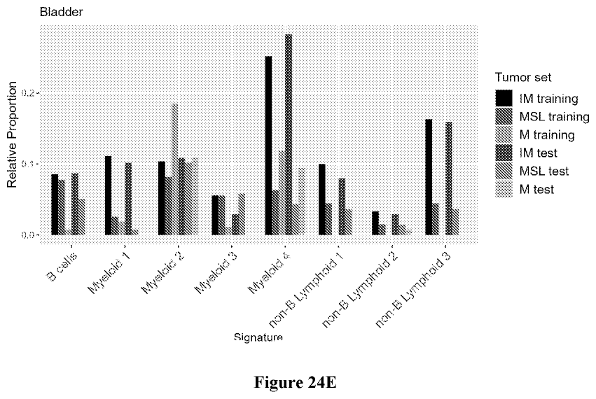

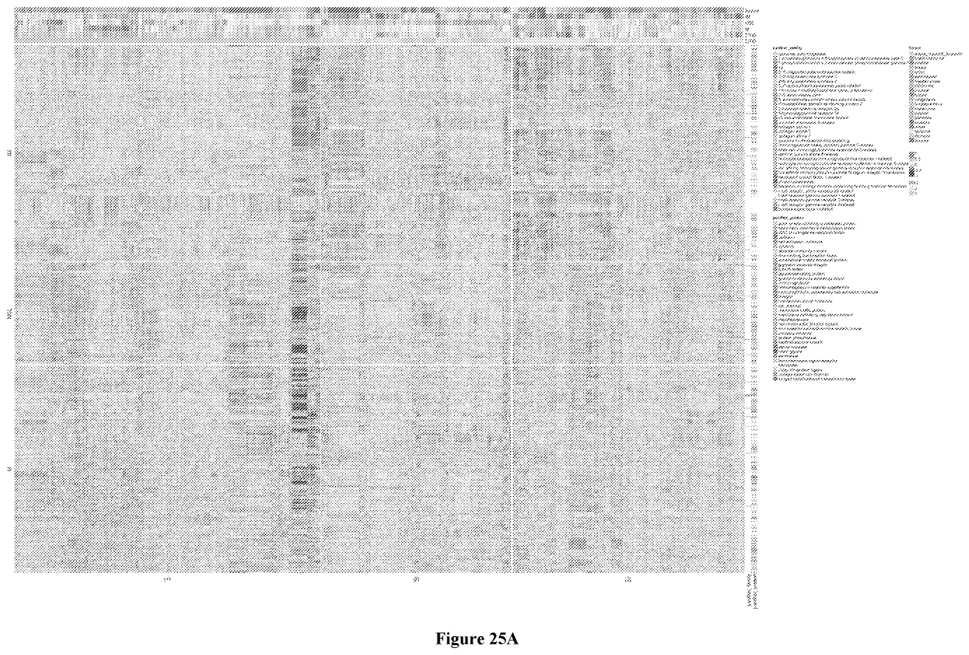

To examine the variation in the prevalence of the tumor samples with high network signature score between tissues and TIME subtypes, the relative proportion of tumor samples have a network signature score higher than the 70th percentile were determined. The samples for a tumor set from The Cancer Genome Atlas (TCGA) were defined as IM, MSL or M subtypes using the 101 gene centroid, as previously described (Ring, Hout et al. 2016). The tumor data was further refined as belonging to the 733 breast samples used for model derivation (training set) or the 359 breast samples reserved as a test set (FIG. 24A), the 350 lung adenocarcinoma samples used for model derivation (training set) or the 165 lung adenocarcinoma tumors reserved as a test set (FIG. 24B), the 352 lung squamous cell carcinoma samples used for model derivation (training set) or the 149 lung squamous cell carcinomas reserved as a test set (FIG. 24C), the 299 colon carcinoma samples used for model derivation (training set) or the 157 colon carcinoma samples reserved as a test set (FIG. 24D), and the 270 bladder carcinoma samples used for model derivation (training set) or the 138 bladder carcinoma samples reserved as a test set (FIG. 24E).

Similar proportions of positive samples were seen in both training and test sets in the IM, MSL and M subsets, indicating that the models give consistent results. In adenocarcinoma of the lung, the non-B lymphoid signature 1 had proportions of 0.446 and 0.406 in the IM train and test sets, and 0.271 and 0.255 in the MSL train and test sets (and 0 in both the M subsets). Similarly, the B lymphoid signature had proportions of 0.489 and 0.473 in the IM train and test sets, and 0.257 and 0.194 in the MSL train and test sets (and <0.01 in both the M subsets). In breast carcinoma, the non-B lymphoid signature 1 had proportions of 0.117 and 0.111 in the IM train and test sets, 0.138 and 0.167 in the MSL train and test sets, and 0.027 and 0.028 the M subsets. Similarly, the B lymphoid signature in breast had proportions of 0.109 and 0.106 in the IM train and test sets, 0.147 and 0.175 in the MSL train and test sets, and 0.046 and 0.047 the M subsets.

However, different proportions were observed between TME subtypes, and between tissues, suggesting that different tumors have different populations of infiltrating immune cells. As expected, IM subtype samples had the greatest prevalence of immune signatures, and M the least. The myeloid signatures frequently had a strong presence in MSL subtype samples, and this relatively increased prevalence also showed variation between tissues. In adenocarcinoma of the lung (FIG. 24B), the myeloid 1 signature had a prevalence of 0.251 and 0.218 (train and test) in the MSL subtype, while in lung squamous cell carcinoma the proportions were much lower, at 0.085 and 0.087 (train and test) in the MSL subtype samples. Among other things, this example demonstrates that the TME has a strong role in determining tumor immune infiltrate composition.

### Distribution of the Network Signatures in Relation to the Tumor Immune Microenvironment

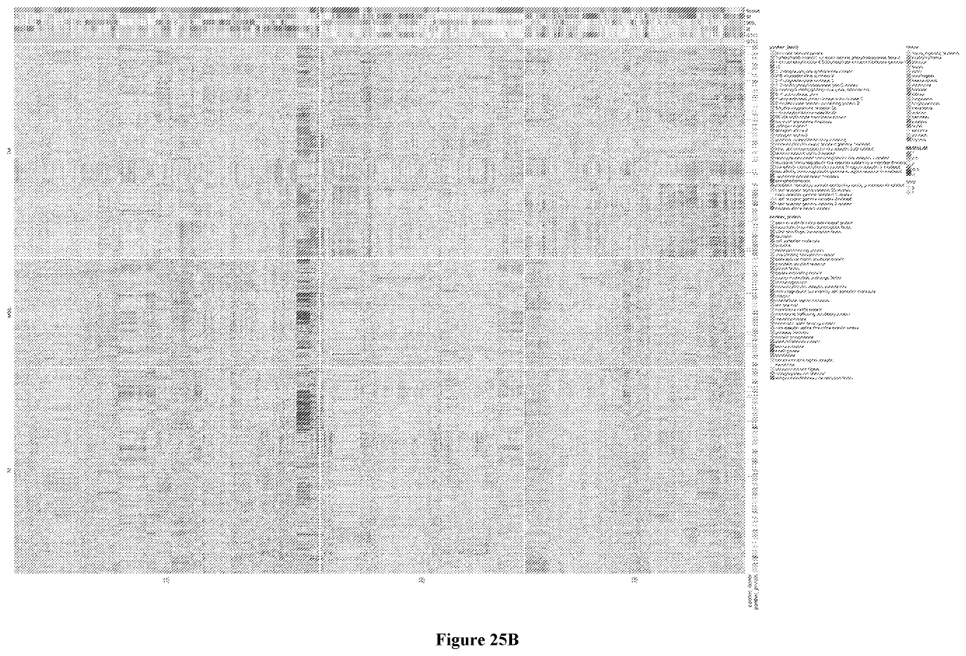

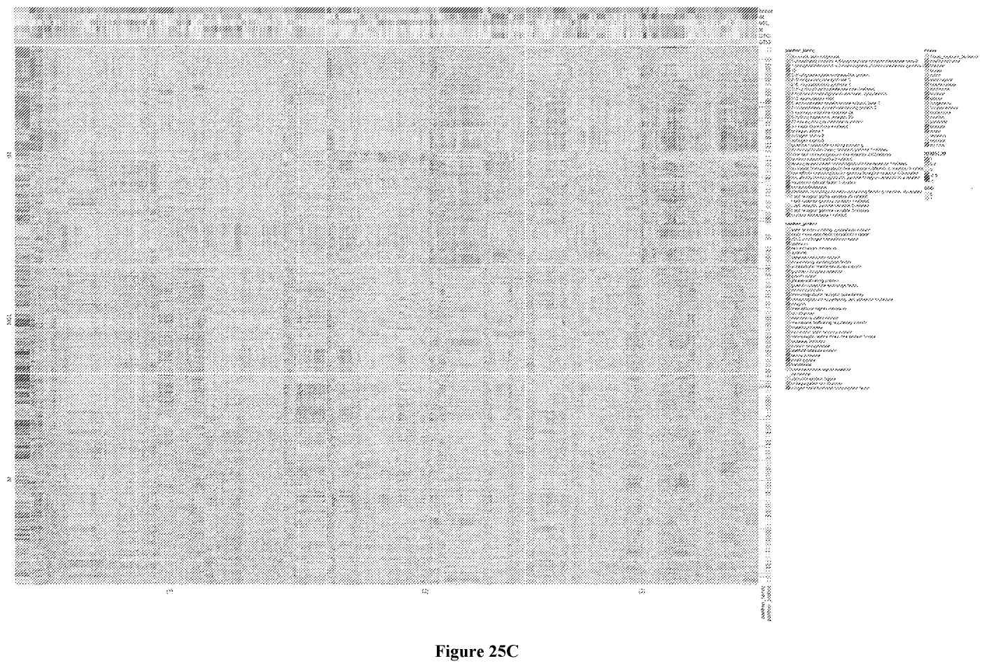

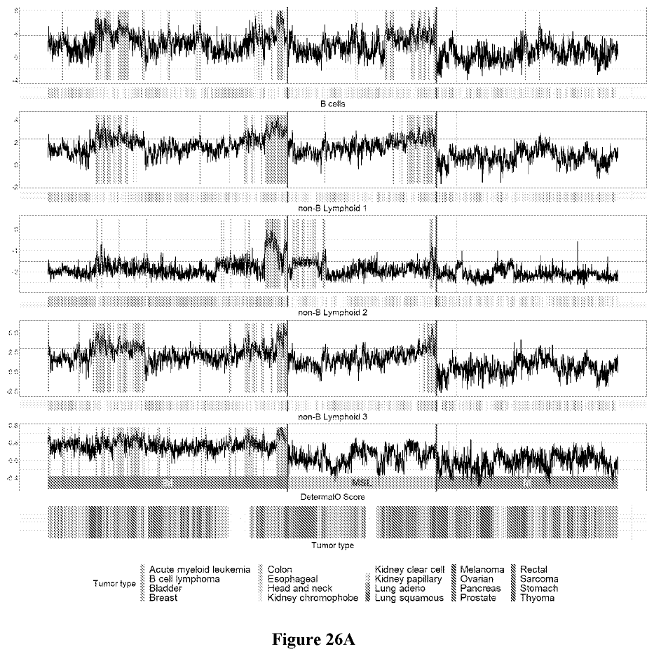

To describe the variation in gene expression and distribution of tumor types in relation to the tumor immune microenvironment, we clustered tumor samples comprising 20 tumor types from The Cancer Genome Atlas (TCGA; https://portal.gdc.cancer.gov/) using the training set of cases. A list of 2,967 genes derived (as described herein) to highlight distinct molecular physiologies across the TIME expression landscape were used for clustering. Genes and tumor samples were initially grouped by their correlation to the IM, MSL and M tumor subtypes, and then hierarchically clustered. Annotation of genes by protein families is shown, and each tumor sample is annotated with the correlation to the IM, MSL and M subtypes using the 101 gene centroid models, as well as the 27 gene DTIO score and call. Clusters using the 4,883 training samples (FIG. 25A), the 2,279 test samples (FIG. 25B), and the complete set of 7,162 samples (FIG. 25C) are shown.

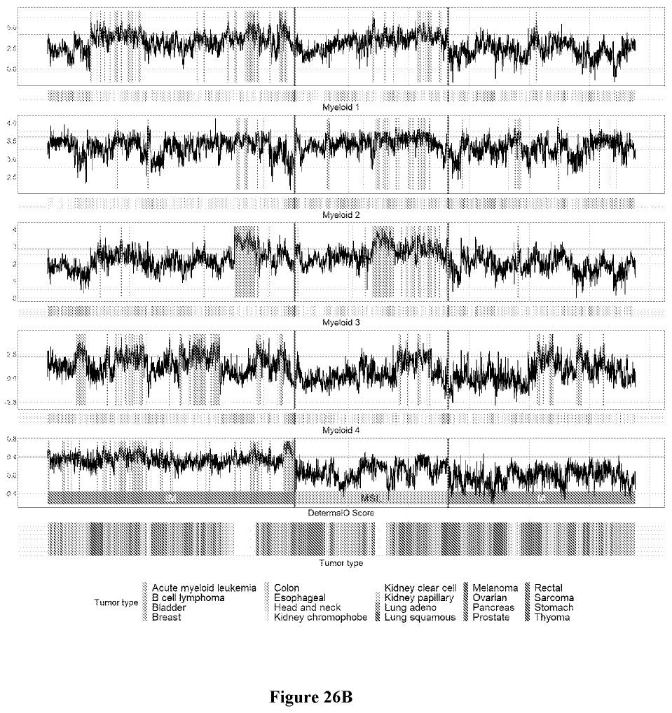

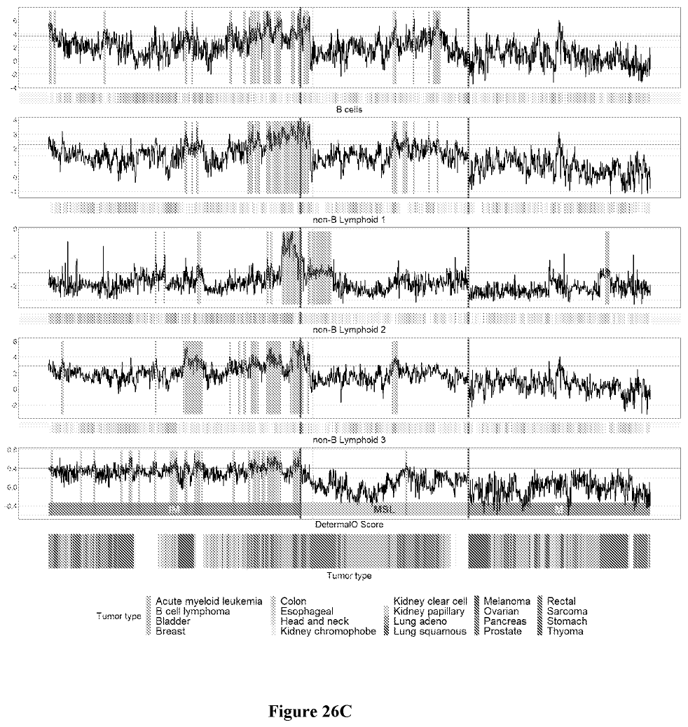

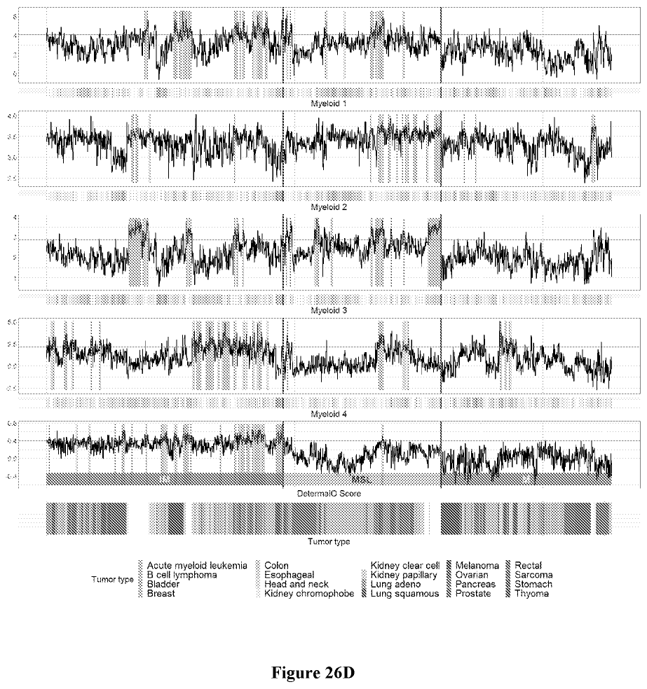

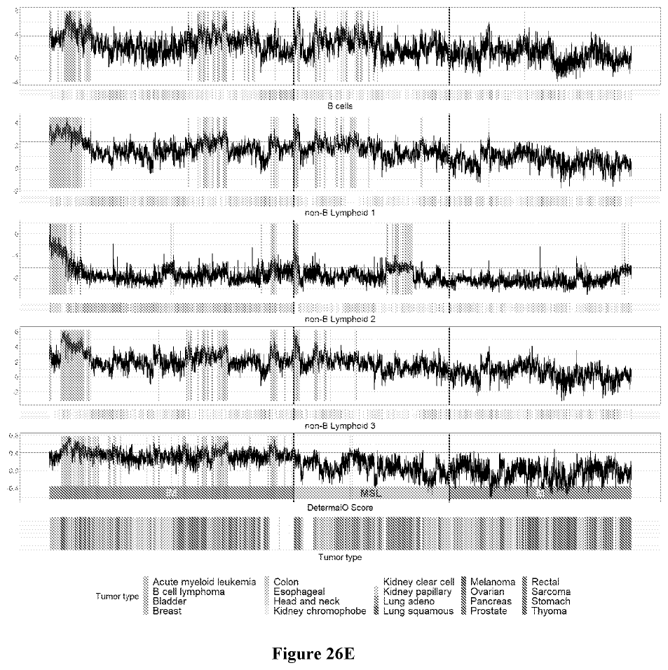

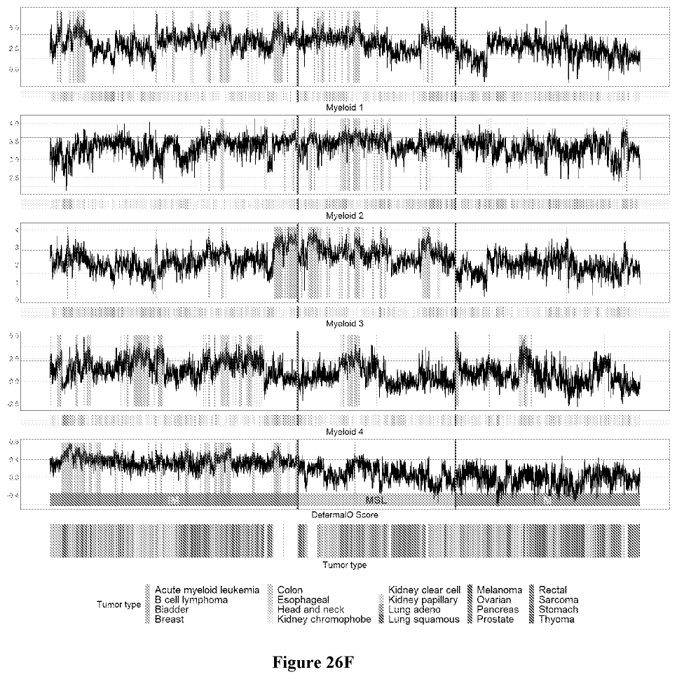

The distribution of the lymphoid and myeloid network signatures in relation to these clusters is shown for the training samples (FIG. 26A and FIG. 26B), the test samples (FIG. 26C and FIG. 26D), and all samples (FIG. 26E and FIG. 26F). The score of the network signatures is shown, and large peaks above their mean value are highlighted. To identify groups of tumors in which DTIO may not be capturing information about immune cell infiltrate presences, the degree of agreement for the signature and DTIO, defined as the min-max normalization of the network score subtracted from the min-max normalization of the DTIO score, was also shown.

There is considerable variation between the network signatures across the TIME. The B cell, and non-B lymphoid signatures 1 and 3 show an overall similarity, but non-B lymphoid signature is relatively distinct, especially in the MSL subset. The Pearson correlation of the B lymphoid signature with DTIO across the training and set samples was 0.46 and 0.45, in non-B lymphoid 1 it was 0.51 and 0.50, and in non-B lymphoid 3 it was 0.64 and 0.63, while in non-B lymphoid 2 it was 0.38 and 0.37 (train and test sets). They myeloid signature showed even greater differences with DTIO, with myeloid 2 and 3 have correlations with DTIO of −0.06 and −0.01 (train and test) and 0.099 and 0.12 (train and test), respectively.

Association of Network Signatures with Outcome in ICI Treated Patients

To compare the association of outcome of several immune signatures, all derived ImmGen and network signatures, as well as the immune signatures from the xCell study, were applied to cohort 2 (platinum treated) of the Imvigor 210 trial (Suzman, Agrawal et al. 2019). Significant signatures are shown in Table 44, and were assessed on a bladder cohort (GSE176307) (Rose, Weir et al. 2021), and a melanoma cohort (GSE91061) (Riaz, Havel et al. 2017). The melanoma cohort was analyzed both before treatment with Nivolumab and after four weeks of treatment. Of the immune signatures that were significant on the Imvigor 210 cohort, only DTIO and the two network signatures (B cell signature and non-B lymphoid signature 3) were significant on the bladder and melanoma cohorts. It is interesting that the only xCell signatures to be significant in the Imvigor cohort, and are also significant in the melanoma cohort, are T cell signatures, which might have a relationship with the network model non-B Lymphoid 3, and plasma cells, and may reflect the significance of the network B cell signature.

TABLE 44
Significant immune infiltrate signatures from Imvigor 210 trial data.

| | Imvigor 210 cohort 2 | | | GSE176307 | | | GSE91061 Pre−treatment | | | GSE91061 On treatment | | |
|---|---|---|---|---|---|---|---|---|---|---|---|---|
| | (bladder, 2 year OS) | | | (bladder, 1 year OS) | | | (melanoma, response) | | | (melanoma, response) | | |
| Signature | N | OR (95% CI) | P value | N | beta (95% CI) | P value | N | beta (95% CI) | P value | N | beta (95% CI) | P value |
| DTIO | 272 | 0.28 | <0.0001 | 89 | 0.02 | 0.0115 | 49 | 1.89 | 0.0952 | 56 | 2.72 | 0.0215 |
| | | (0.15-0.5) | | | (0-0.39) | | | (−1.82-5.6) | | | (−2.61-8.06) | |
| Network: B cells | 272 | 0.91 | 0.05 | 89 | 0.8 | 0.0218 | 49 | 0.22 | 0.1051 | 56 | 0.5 | 0.0005 |
| | | (0.83-1) | | | (0.67-0.97) | | | (−0.21-0.64) | | | (−0.48-1.48) | |
| Network: non-B | 272 | 0.87 | 0.01 | 89 | 0.72 | 0.0051 | 49 | 0.29 | 0.0804 | 56 | 0.53 | 0.0029 |
| Lymphoid 3 | | (0.78-0.97) | | | (0.57-0.9) | | | (−0.28-0.85) | | | (−0.51-1.57) | |
| ImmGen: CD8+ | 272 | 1.34 | 0.01 | 89 | 1.15 | 0.351 | 49 | 0.1 | 0.8155 | 56 | 0.28 | 0.3943 |
| activated T cells | | (1.06-1.68) | | | (0.86-1.55) | | | (−0.09-0.29) | | | (−0.27-0.84) | |
| ImmGen: | 272 | 1.5 | 0 | 89 | 0.78 | 0.2788 | 49 | −0.22 | 0.3759 | 56 | −0.01 | 0.9606 |
| Macrophage | | (1.2-1.88) | | | (0.5-1.22) | | | (0.22-−0.66) | | | (0.01-−0.04) | |
| xCell: CD8+ | 272 | <0.001 | 0.05 | 89 | <0.001 | 0.203 | 49 | 7.49 | 0.0482 | 56 | 6.49 | 0.0029 |
| central memory | | (<0.001-0.88) | | | (<0.001->100) | | | (−7.19-22.16) | | | (−6.23-19.21) | |
| T cell | | | | | | | | | | | | |
| xCell: CD8+ | 272 | <0.001 | 0.04 | 89 | <0.001 | 0.4258 | 49 | 31.52 | 0.1167 | 56 | 17.84 | 0.0019 |
| effector memory | | (<0.001-0.4) | | | (<0.001->100) | | | (−30.26-93.3) | | | (−17.13-52.82) | |
| T cell | | | | | | | | | | | | |
| xCell: Plasma | 272 | <0.001 | 0.04 | 89 | <0.001 | 0.1822 | 49 | 18.93 | 0.4096 | 56 | 47.42 | 0.0232 |
| cells | | (<0.001-0.54) | | | (<0.001->100) | | | (−18.17-56.04) | | | (−45.53->100) | |
| xCell: T gamma- | 272 | <0.001 | 0 | 89 | <0.001 | 0.1026 | 49 | 29.37 | 0.1259 | 56 | 32.25 | 0.0214 |
| delta cells | | (<0.001-0) | | | (<0.001->100) | | | (−28.19-86.93) | | | (−30.96-95.47) | |

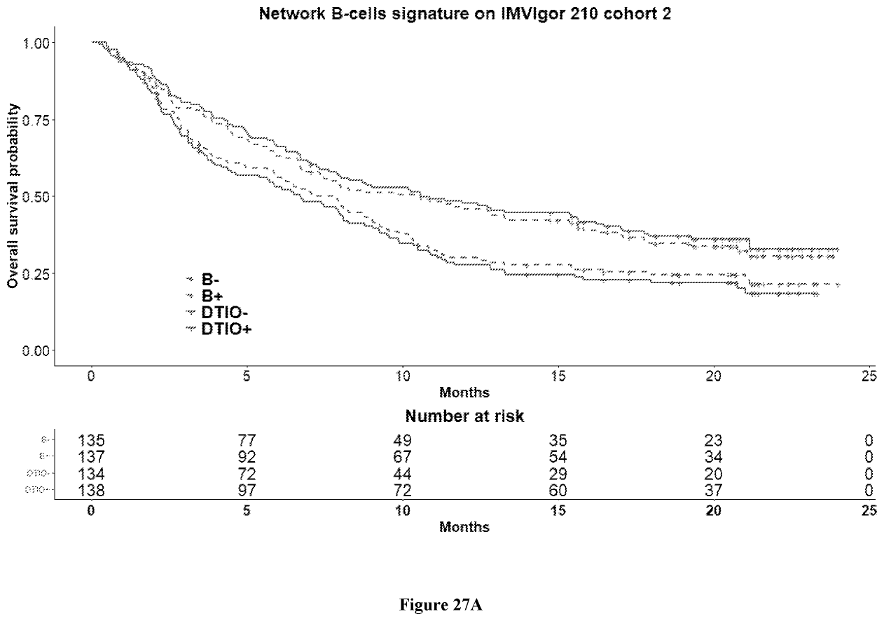

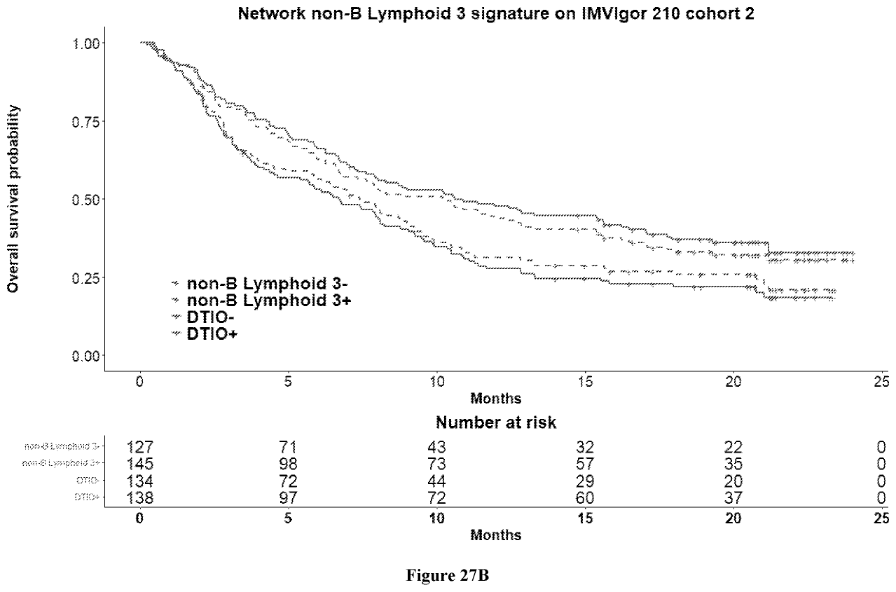

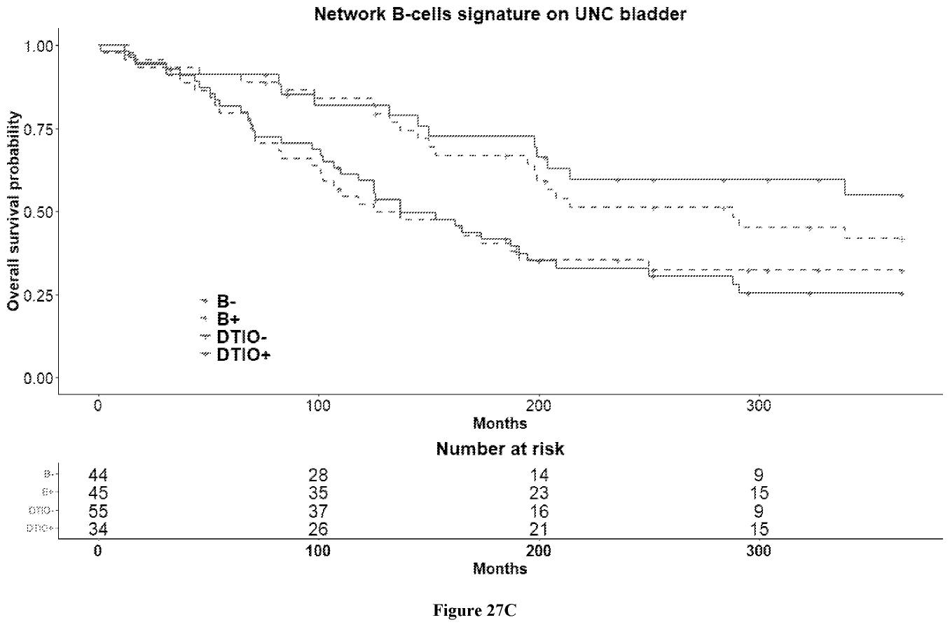

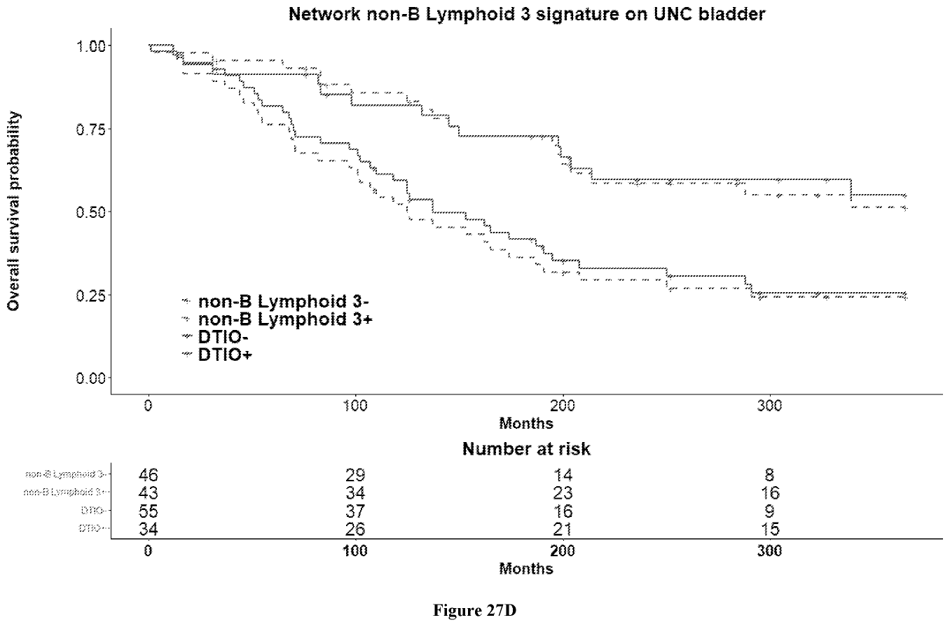

The similar prognostic capabilities of DTIO and these two network signatures can be observed in Kaplan-Meier plots of the overall survival of patients with high network B cell signature and non-B lymphoid 3 scores (defined as above or below the mean of the study population) was observed for two year overall survival in Imvigor (N=272) (FIG. 27A and FIG. 27B) and the other bladder cohort (GSE176307, N=89) (FIG. 27C and FIG. 27D. As expected from the similarity in distributions of scores of these signatures with DTIO, each captures much of the prognostic ability of DTIO.

Among other things, the present example demonstrates methods for identification of novel immune infiltrate populations within tumor samples (e.g., within tumor immune microenvironment (TIME)). In some embodiments, methods provided herein may be used to determine immune infiltrate levels for a particular tumor type without the need for a solid tumor biopsy. In some embodiments, immune infiltrate information provided herein may be used to inform or select one or more therapies for a tumor. In some embodiments, immune infiltrate information provided herein may be used in combination with tumor gene expression subtype data and/or DetermaIO scoring to inform or select one or more therapies for a tumor. In some embodiments, immune infiltrate information provided herein may be used in combination with tumor gene expression subtype data and/or DetermaIO scoring to identify patients whose cancer may not be adequately met by existing therapeutic regimens, or otherwise are strong candidates for novel drug discovery and development programs.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of treating cancer, the method comprising a step of:
administering immunomodulation therapy to subjects whose tumors have been determined to be responsive to the immunomodulation therapy by assessment of both:
(i) subtype markers of a subtype selected from mesenchymal (M), mesenchymal stem-like (MSL), and combinations thereof; and
(ii) status markers of immunomodulatory (IM) status;
wherein the subtype markers are considered to indicate likely non-responsiveness to immunomodulation therapy and the status markers are considered to indicate likely responsiveness to immunomodulation therapy.

2. A method of assessing a tumor's likely responsiveness to immunomodulation therapy, which method comprises
(a) assessing both:
(i) subtype markers of a subtype selected from mesenchymal (M), mesenchymal stem-like (MSL), and combinations thereof; and
(ii) status markers of immunomodulatory (IM) status; and
(b) calculating, by means of a computer, an IO score by weighting the subtype markers as likely to indicate non-responsiveness to immunomodulation therapy and the status markers as likely to indicate responsiveness to immunomodulation therapy.

3. The method of claim 2, further comprising a step of administering the immunomodulation therapy to a subject whose tumor has been determined to have an IO score above a threshold established to correlate with responsiveness to the immunomodulation therapy.

4. The method of claim 2, further comprising a step of administering an alternative therapy to a subject whose tumor has been determined to have an IO score below a certain threshold.

5. The method of claim 3, wherein the immunomodulation therapy is selectively administered to subjects whose tumors have been determined to have IO scores above a certain threshold.

6. The method of claim 3, wherein the immunomodulation therapy is selected from the ICI therapy, CAR-T cell therapy, neoantigen vaccine therapy, or combinations thereof.

7. The method of claim 4, wherein the alternative therapy is kinase inhibitor or other tumor microenvironment modulating therapy.

8. A method of monitoring therapy administered to a cancer patient, the method comprising steps of:
(a) at each of a plurality of time points, determining both
(i) subtype markers of a subtype selected from mesenchymal (M), mesenchymal stem-like (MSL), and combinations thereof; and
(ii) status markers of immunomodulatory (IM) status;
wherein the subtype markers are considered to indicate likely non-responsiveness to immunomodulation therapy and the status markers are considered to indicate likely responsiveness to immunomodulation therapy, so that an IO score representing the patient's likelihood of responding to the immunomodulation therapy is determined; and
(b) adjusting therapy in light of a change in the IO score.

9. A method of treating a tumor, which method comprises steps of:
(a) at a first time point, assessing the tumor by determining both
(i) subtype markers of a subtype selected from mesenchymal (M), mesenchymal stem-like (MSL), and combinations thereof; and
(ii) status markers of immunomodulatory (IM) status;
wherein the subtype markers are considered to indicate likely non-responsiveness to immunomodulation therapy and the status markers are considered to indicate likely responsiveness to immunomodulation therapy, so that an IO score representing the tumor's likelihood of responding to the immunomodulation therapy is determined;
(b) selecting therapy according to the IO score, wherein the selecting comprises:
(i) initiating or continuing immunomodulation therapy when the IO score meets a threshold determined to correlate with responsiveness to the immunomodulation therapy; and/or
(ii) reducing or withdrawing the immunomodulation therapy and/or initiating or continuing alternative therapy when the IO score meets a threshold determined to correlate with non-responsiveness to the immunomodulation therapy.

10. The method of claim 8, wherein an increase in IO score, or an IO score greater than a predefined threshold, indicates an increased likelihood of responding to the immunomodulation therapy.

11. The method of claim 8, wherein a decrease in IO score, or an IO score less than a predefined threshold, indicates a reduced likelihood of responding to the immunomodulation therapy.

12. The method of claim 8, wherein the immunomodulation therapy is selected from the ICI therapy, CAR-T cell therapy, neoantigen vaccine therapy, or combinations thereof.

13. The method of claim 8, wherein the alternative therapy is kinase inhibitor therapy.

14. A method comprising steps of:
a. receiving, by a processor of a computing device, data corresponding to levels of a plurality of markers for each of:
i. a subtype selected from M, MSL, and combinations thereof; and
ii. an IM status;
b. automatically determining, by the processor, a classification of the subject as non-responsive to a first therapy (e.g. immunomodulation therapy) using the data received in step (a) to produce a numerical score; and, optionally,
c. prescribing and/or administering a second therapy (e.g. an alternative to the first therapy, e.g., an alternative to immunomodulation therapy) to the subject for treatment of the disease, thereby avoiding prescription and/or administration of the first therapy to the subject.

15. A method comprising the steps of:
a. receiving, by a processor of a computing device, data corresponding to levels of a plurality of markers for each of:
i. a subtype selected from M, MSL, and combinations thereof; and
ii. an IM status;
b. automatically determining, by the processor, a classification of the subject as responsive to a first therapy (e.g. immunomodulation therapy) using the data received in step (a) to produce a numerical score; and, optionally,
c. prescribing and/or administering the first therapy to the subject for treatment of the disease.

16. In a method of administering an immunomodulation therapy, the improvement that comprises administering the therapy selectively to subjects who have been assigned a numerical IO score calculated through assessment of each of:
a. Mesenchymal (M) subtype and/or mesenchymal stem-like (MSL) subtype as a negative predictor of responsiveness; and
b. IM status as a positive predictor of responsiveness.

17. The method of claim 16, wherein the assigned IO score is above a threshold established to distinguish between responsive and non-responsive historical subjects who have received the immunomodulation therapy.

18. A method of determining a tumor classifier effective to distinguish between responsiveness and non-responsiveness to immunomodulation therapy, the method comprising steps of:
a. Employing elastic net regularized linear models to create individual subclassifying models for a set of subtypes;
b. Training the classifier on a gene expression dataset from a sample of interest; and
c. Assessing the correlation between the classifier and responsiveness to immunomodulation therapy.

19. The method of claim 18, wherein the classifier comprises a set of between 75 and 100 genes.

20. The method of claim 18, wherein the classifier comprises a set of between 50 and 75 genes.

21. The method of claim 18, wherein the classifier comprises a set of between 25 and 50 genes.

22. The method of claim 18, wherein the classifier comprises a set of less than 25 genes.

23. The method of claim 18, wherein the subtypes are defined based upon previously established models.

24. The method of claim 23, wherein the classifier comprises a reduced gene set compared to previously established models.

25. A method of treating cancer, the method comprising steps of:
(i) assessing expression levels for one or more genes selected from the group consisting of:
CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, SAMSN1, CD80, CLEC7A, PDCD1LG2, CD274, S100A8, KYNU, LINC02195, IL9R, DUSP5, TNFAIP8, TNFSF10, RARRES3, APOL3, LINC02446, ZNF683, IFNG, FASLG, CD48, CD52, C16orf54, TESPA1, JAML, GMFG, ARHGAP15, TMEM273, CD3G, TIGIT, SIRPG, TRAC, CD3E, CD2, TRBV28, CD3D, TRBC2, CCR5, CD8A, CCL5, IL2RB, CXCR6, KMO, SNX10, PIK3AP1, SLC7A7, VCAM1, RASSF4, TFEC, HAVCR2, APOL6, IDO1, CXCL9, GBP5, GBP1, GBP4, CXCL11, CXCL10, LAP3, STAT1, WARS1, SAMHD1, ZBP1, OASL, EPSTI1, IL15RA, USP30-AS1, BATF2, ETV7, PSMB10, RTP4, CARD16, GZMB, GZMH, GNLY, CD8B, CTSW, CST7, NKG7, GZMA, PRF1, CD247, SLA2, PDCD1, CD7, LAG3, HNRNPA1P21, FOXP3, CCR8, CXCL13, AIM2, IL2RA, ICOS, CTLA4, TNFRSF9, IL21R, BTN3A3, BTN3A1, TAP2, NLRC5, HLA-F, PSMB8, PSMB9, TAP1, HCP5, UBE2L6, PSME2, IRF1, C19orf38, IGFLR1, LINC01943, RAB33A, SLC2A6, IFI30, LILRB3, IL23A, PSME2P2, ITGAE, STAC3, FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1, ADH1B, C7, CCL14, SELP, ACKR1, MMRN1, ITM2A, AQP1, ABI3BP, P2RY12, MPRIP, KIF13B, FYCO1, SPTLC2, ADGRA3, RBFOX2, ITGB4, KRT17, KRT16, KRT14, KRT5, DSG3, COL17A1, TMEM119, PODN, SVEP1, LAMA2, COL14A1, FGF7, OGN, PRELP, ELN, MFAP4, SSC5D, PTGDS, CHRDL1, ITGBL1, ASPN, PDGFRB, HTRA1, HEG1, ZCCHC24, SGCD, SRPX, APOD, SHC4, MIA, IL17D, LRRN4CL, BOC, PDZRN3, SFRP1, TCF7L1, CACNA1G, SPEG, COL2A1, CRISPLD1, PIANP, NACAD, EFNB3, PCYT1B, RGMA, GLI2, PCDH19, ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MIDI, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, and ZCCHC24;
(ii) comparing the assessed expression with a set of reference thresholds for the one or more genes; and
(iii) administering ICI therapy to the subject if the comparing determines that the assessed expression levels have a significant pattern relative to their reference thresholds.

26. A method of assessing a tumor's likely responsiveness to immunomodulation therapy, which method comprises
(a) assessing both:
(i) subtype markers of a subtype selected from mesenchymal (M), mesenchymal stem-like (MSL), and combinations thereof; and
(ii) status markers of immunomodulatory (IM) status; and
(b) calculating, by means of a computer, an IO score by weighting the subtype markers as likely to indicate non-responsiveness to immunomodulation therapy and the status markers as likely to indicate responsiveness to immunomodulation therapy;
wherein the subtype markers and status markers are expression levels for a set of genes selected from the group consisting of:
CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNF AIP8, TNFSF10, COL2A1, FOXC1, KRT16, MIA, SFRP1, APOD, ASPN, HTRA1, CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, SAMSN1, CD80, CLEC7A, PDCD1LG2, CD274, S100A8, KYNU, LINC02195, IL9R, DUSP5, TNFAIP8, TNFSF10, RARRES3, APOL3, LINC02446, ZNF683, IFNG, FASLG, CD48, CD52, C16orf54, TESPA1, JAML, GMFG, ARHGAP15, TMEM273, CD3G, TIGIT, SIRPG, TRAC, CD3E, CD2, TRBV28, CD3D, TRBC2, CCR5, CD8A, CCL5, IL2RB, CXCR6, KMO, SNX10, PIK3AP1, SLC7A7, VCAM1, RASSF4, TFEC, HAVCR2, APOL6, IDO1, CXCL9, GBP5, GBP1, GBP4, CXCL11, CXCL10, LAP3, STAT1, WARS1, SAMHD1, ZBP1, OASL, EPSTI1, IL15RA, USP30-AS1, BATF2, ETV7, PSMB10, RTP4, CARD16, GZMB, GZMH, GNLY, CD8B, CTSW, CST7, NKG7, GZMA, PRF1, CD247, SLA2, PDCD1, CD7, LAG3, HNRNPA1P21, FOXP3, CCR8, CXCL13, AIM2, IL2RA, ICOS, CTLA4, TNFRSF9, IL21R, BTN3A3, BTN3A1, TAP2, NLRC5, HLA-F, PSMB8, PSMB9, TAP1, HCP5, UBE2L6, PSME2, IRF1, C19orf38, IGFLR1, LINC01943, RAB33A, SLC2A6, IFI30, LILRB3, IL23A, PSME2P2, ITGAE, STAC3, FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1, ADH1B, C7, CCL14, SELP, ACKR1, MMRN1, ITM2A, AQP1, ABI3BP, P2RY12, MPRIP, KIF13B, FYCO1, SPTLC2, ADGRA3, RBFOX2, ITGB4, KRT17, KRT16, KRT14, KRT5, DSG3, COL17A1, TMEM119, PODN, SVEP1, LAMA2, COL14A1, FGF7, OGN, PRELP, ELN, MFAP4, SSC5D, PTGDS, CHRDL1, ITGBL1, ASPN, PDGFRB, HTRA1, HEG1, ZCCHC24, SGCD, SRPX, APOD, SHC4, MIA, IL17D, LRRN4CL, BOC, PDZRN3, SFRP1, TCF7L1, CACNA1G, SPEG, COL2A1, CRISPLD1, PIANP, NACAD, EFNB3, PCYT1B, RGMA, GLI2, PCDH19, ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MIDI, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, ZCCHC24, and combinations thereof.

27. The method of claim 26, wherein the subtype markers and status markers comprise at least one gene from one or more gene groups below:
Group A: CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1;
Group B1: CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10;
Group B2: COL2A1, FOXC1, KRT16, MIA, SFRP1;
Group B3: APOD, ASPN, HTRA1;
Group C1: SAMSN1, CD80, CLEC7A, PDCD1LG2, CD274, S100A8, KYNU, LINC02195, IL9R, DUSP5;
Group C2: TNFAIP8, TNFSF10;
Group C3: RARRES3, APOL3, LINC02446, ZNF683, IFNG, FASLG;
Group C4: CD48, CD52, C16orf54, TESPA1, JAML, GMFG, ARHGAP15, TMEM273;
Group C5: CD3G, TIGIT, SIRPG, TRAC, CD3E, CD2, TRBV28, CD3D, TRBC2, CCR5, CD8A, CCL5, IL2RB, CXCR6;
Group C6: KMO, SNX10, PIK3AP1, SLC7A7, VCAM1, RASSF4, TFEC, HAVCR2;
Group C7: APOL6, IDO1, CXCL9, GBP5, GBP1, GBP4, CXCL11, CXCL10, LAP3, STAT1, WARS1, SAMHD1;
Group C8: ZBP1, OASL, EPSTI1, IL15RA, USP30-AS1, BATF2, ETV7, PSMB10, RTP4, CARD16;
Group C9: GZMB, GZMH, GNLY, CD8B, CTSW, CST7, NKG7, GZMA, PRF1, CD247, SLA2, PDCD1, CD7, LAG3;
Group C10: HNRNPA1P21, FOXP3, CCR8, CXCL13, AIM2, IL2RA, ICOS, CTLA4, TNFRSF9, IL21R;
Group C11: BTN3A3, BTN3A1, TAP2, NLRC5, HLA-F, PSMB8, PSMB9, TAP1, HCP5, UBE2L6, PSME2, IRF1;
Group C12: C19orf38, IGFLR1, LINC01943, RAB33A, SLC2A6, IFI30, LILRB3, IL23A, PSME2P2, ITGAE, STAC3;
Group C13: FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1;
Group C14: ADH1B, C7, CCL14, SELP, ACKR1, MMRN1, ITM2A, AQP1, ABI3BP, P2RY12;
Group C15: MPRIP, KIF13B, FYCO1, SPTLC2, ADGRA3, RBFOX2;
Group C16: ITGB4, KRT17, KRT16, KRT14, KRT5, DSG3, COL17A1;
Group C17: TMEM119, PODN, SVEP1, LAMA2, COL14A1, FGF7, OGN, PRELP, ELN, MFAP4, SSC5D, PTGDS, CHRDL1;
Group C18: ITGBL1, ASPN, PDGFRB, HTRA1, HEG1;
Group C19: ZCCHC24, SGCD, SRPX, APOD, SHC4, MIA, IL17D, LRRN4CL, BOC, PDZRN3, SFRP1;
Group C20: TCF7L1, CACNA1G, SPEG, COL2A1, CRISPLD1, PIANP, NACAD, EFNB3, PCYT1B, RGMA, GLI2, PCDH19; and
Group D1: ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MID1, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, ZCCHC24.

28. The method of claim 27, wherein the subtype markers and status markers comprise at least one gene from five or more of the gene groups.

29. The method of claim 28, wherein the subtype markers and status markers comprise at least one gene from ten or more of the gene groups.

30. The method of claim 29, wherein the subtype markers and status markers comprise at least one gene each of the gene groups.

31. The method of claim 27, wherein the subtype markers and status markers comprise:
(i) at least one gene selected from Group A;
(ii) at least one gene selected from any one of Group B1, Group B2, or Group B3;
(iii) at least one gene selected from any one of Group C1, Group C2, Group C3, Group C4, Group C5, Group C6, Group C7, Group C8, Group C9, Group C10, Group C11, Group C12, Group C13, Group C14, Group C15, Group C16, Group C17, Group C18, Group C19, Group C20; and
(iv) at least one gene selected from Group D1.

32. The method of claim 2, further comprising a step of administering an additional therapy to a subject whose tumor has been determined to have an IO score below a certain threshold.

33. The method of claim 32, wherein the additional therapy is selected to target gene pathways associated with negative IO scores.

34. The method of claim 33, wherein the immunomodulation therapy is ICI therapy and the additional therapy is not ICI therapy.

35. The method of claim 33, wherein the immunomodulation therapy and additional therapy are co-administered.

36. The method of claim 33, wherein the immunomodulation therapy and additional therapy are administered sequentially.

37. The method of claim 4, wherein the alternative therapy is selected to target gene pathways associated with negative IO scores.

38. The method of claim 37, wherein the immunomodulation therapy is ICI therapy and the alternative therapy is not ICI therapy.

39. The method of claim 37, wherein:
(i) the alternative therapy is administered; and
(ii) the IO score is determined after alternative therapy administration;
wherein, if the IO score has changed to be above a certain threshold, the alternative therapy is either:
discontinued in favor of immunomodulation therapy; or
continued along with co-administration of immunomodulation therapy.

40. A method of establishing a biomarker indicative of immune microenvironment status, the method comprising steps of:
determining a correlation between a candidate biomarker and one or more of IM status markers and M and MSL subtype markers;
incorporating the candidate biomarker into a complete biomarker that includes both indicators of likely responsiveness and indicators of likely non-responsiveness to immunomodulation therapy.

41. The method of claim 40, wherein the IM status markers and the M and MSL subtype markers comprise at least one gene from one or more gene groups below:
Group A: CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1;
Group B1: CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNF AIP8, TNFSF10;
Group B2: COL2A1, FOXC1, KRT16, MIA, SFRP1;
Group B3: APOD, ASPN, HTRA1;
Group C1: SAMSN1, CD80, CLEC7A, PDCD1LG2, CD274, S100A8, KYNU, LINC02195, IL9R, DUSP5;
Group C2: TNFAIP8, TNFSF10;
Group C3: RARRES3, APOL3, LINC02446, ZNF683, IFNG, FASLG;
Group C4: CD48, CD52, C16orf54, TESPA1, JAML, GMFG, ARHGAP15, TMEM273;
Group C5: CD3G, TIGIT, SIRPG, TRAC, CD3E, CD2, TRBV28, CD3D, TRBC2, CCR5, CD8A, CCL5, IL2RB, CXCR6;
Group C6: KMO, SNX10, PIK3AP1, SLC7A7, VCAM1, RASSF4, TFEC, HAVCR2;
Group C7: APOL6, IDO1, CXCL9, GBP5, GBP1, GBP4, CXCL11, CXCL10, LAP3, STAT1, WARS1, SAMHD1;
Group C8: ZBP1, OASL, EPSTI1, IL15RA, USP30-AS1, BATF2, ETV7, PSMB10, RTP4, CARD16;
Group C9: GZMB, GZMH, GNLY, CD8B, CTSW, CST7, NKG7, GZMA, PRF1, CD247, SLA2, PDCD1, CD7, LAG3;
Group C10: HNRNPA1P21, FOXP3, CCR8, CXCL13, AIM2, IL2RA, ICOS, CTLA4, TNFRSF9, IL21R;
Group C11: BTN3A3, BTN3A1, TAP2, NLRC5, HLA-F, PSMB8, PSMB9, TAP1, HCP5, UBE2L6, PSME2, IRF1;
Group C12: C19orf38, IGFLR1, LINC01943, RAB33A, SLC2A6, IFI30, LILRB3, IL23A, PSME2P2, ITGAE, STAC3;
Group C13: FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1;
Group C14: ADH1B, C7, CCL14, SELP, ACKR1, MMRN1, ITM2A, AQP1, ABI3BP, P2RY12;
Group C15: MPRIP, KIF13B, FYCO1, SPTLC2, ADGRA3, RBFOX2;
Group C16: ITGB4, KRT17, KRT16, KRT14, KRT5, DSG3, COL17A1;
Group C17: TMEM119, PODN, SVEP1, LAMA2, COL14A1, FGF7, OGN, PRELP, ELN, MFAP4, SSC5D, PTGDS, CHRDL1;
Group C18: ITGBL1, ASPN, PDGFRB, HTRA1, HEG1;
Group C19: ZCCHC24, SGCD, SRPX, APOD, SHC4, MIA, IL17D, LRRN4CL, BOC, PDZRN3, SFRP1;
Group C20: TCF7L1, CACNA1G, SPEG, COL2A1, CRISPLD1, PIANP, NACAD, EFNB3, PCYT1B, RGMA, GLI2, PCDH19; and
Group D1: ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MID1, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, ZCCHC24.

42. The method of claim 40, wherein the IM status markers and the M and MSL subtype markers are identified by a gene expression algorithm.

43. The method of claim 40, wherein the biomarker comprises one or more gene variants.

44. The method of claim 43, wherein the one or more gene variants may present differences in gene expression.

45. A method of treating cancer, the method comprising steps of:
(i) assessing expression levels in a sample from a subject suffering from the cancer, for a set of genes selected from the group consisting of:
CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, SAMSN1, CD80, CLEC7A, PDCD1LG2, CD274, S100A8, KYNU, LINC02195, IL9R, DUSP5, TNFAIP8, TNFSF10, RARRES3, APOL3, LINC02446, ZNF683, IFNG, FASLG, CD48, CD52, C16orf54, TESPA1, JAML, GMFG, ARHGAP15, TMEM273, CD3G, TIGIT, SIRPG, TRAC, CD3E, CD2, TRBV28, CD3D, TRBC2, CCR5, CD8A, CCL5, IL2RB, CXCR6, KMO, SNX10, PIK3AP1, SLC7A7, VCAM1, RASSF4, TFEC, HAVCR2, APOL6, IDO1, CXCL9, GBP5, GBP1, GBP4, CXCL11, CXCL10, LAP3, STAT1, WARS1, SAMHD1, ZBP1, OASL, EPSTI1, IL15RA, USP30-AS1, BATF2, ETV7, PSMB10, RTP4, CARD16, GZMB, GZMH, GNLY, CD8B, CTSW, CST7, NKG7, GZMA, PRF1, CD247, SLA2, PDCD1, CD7, LAG3, HNRNPA1P21, FOXP3, CCR8, CXCL13, AIM2, IL2RA, ICOS, CTLA4, TNFRSF9, IL21R, BTN3A3, BTN3A1, TAP2, NLRC5, HLA-F, PSMB8, PSMB9, TAP1, HCP5, UBE2L6, PSME2, IRF1, C19orf38, IGFLR1, LINC01943, RAB33A, SLC2A6, IFI30, LILRB3, IL23A, PSME2P2, ITGAE, STAC3, FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1, ADH1B, C7, CCL14, SELP, ACKR1, MMRN1, ITM2A, AQP1, ABI3BP, P2RY12, MPRIP, KIF13B, FYCO1, SPTLC2, ADGRA3, RBFOX2, ITGB4, KRT17, KRT16, KRT14, KRT5, DSG3, COL17A1, TMEM119, PODN, SVEP1, LAMA2, COL14A1, FGF7, OGN, PRELP, ELN, MFAP4, SSC5D, PTGDS, CHRDL1, ITGBL1, ASPN, PDGFRB, HTRA1, HEG1, ZCCHC24, SGCD, SRPX, APOD, SHC4, MIA, IL17D, LRRN4CL, BOC, PDZRN3, SFRP1, TCF7L1, CACNA1G, SPEG, COL2A1, CRISPLD1, PIANP, NACAD, EFNB3, PCYT1B, RGMA, GLI2, PCDH19, ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MIDI, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, ZCCHC24, and combinations thereof,
wherein reference levels for the set have been established, when considered together, to characterize M, IM and MSL character; and
(ii) comparing the assessed expression levels with the set of established reference levels; and; and
(iii) administering ICI therapy to the subject if the comparing determines that the assessed expression levels indicate that the M, IM, and MSL character of the subject's cancer indicate that it is likely to be responsive to the ICI therapy.

46. A method of establishing a biomarker indicative of immune microenvironment status, the method comprising steps of:
providing a classification system that includes both:
(i) subtype markers of a subtype selected from mesenchymal (M), mesenchymal stem-like (MSL), and combinations thereof; and
(ii) status markers of immunomodulatory (IM) status; and
has been established to predict responsiveness to immunomodulation therapy by considering both markers that indicate likely non-responsiveness and markers that indicate likely responsiveness to the immunomodulation therapy.

47. The method of claim 46, wherein the markers are or comprise expression levels for a set of genes selected from the group consisting of;
CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, CCL5, CD52, CXCL11, CXCL13, DUSP5, GZMB, IDO1, IL23A, ITM2A, KMO, KYNU, PSMB9, PTGDS, RARRES3, RTP4, S100A8, SPTLC2, TNFAIP8, TNFSF10, COL2A1, FOXC1, KRT16, MIA, SFRP1, APOD, ASPN, HTRA1, SAMSN1, CD80, CLEC7A, PDCD1LG2, CD274, S100A8, KYNU, LINC02195, IL9R, DUSP5, TNFAIP8, TNFSF10, RARRES3, APOL3, LINC02446, ZNF683, IFNG, FASLG, CD48, CD52, C16orf54, TESPA1, JAML, GMFG, ARHGAP15, TMEM273, CD3G, TIGIT, SIRPG, TRAC, CD3E, CD2, TRBV28, CD3D, TRBC2, CCR5, CD8A, CCL5, IL2RB, CXCR6, KMO, SNX10, PIK3AP1, SLC7A7, VCAM1, RASSF4, TFEC, HAVCR2, APOL6, IDO1, CXCL9, GBP5, GBP1, GBP4, CXCL11, CXCL10, LAP3, STAT1, WARS1, SAMHD1, ZBP1, OASL, EPSTI1, IL15RA, USP30-AS1, BATF2, ETV7, PSMB10, RTP4, CARD16, GZMB, GZMH, GNLY, CD8B, CTSW, CST7, NKG7, GZMA, PRF1, CD247, SLA2, PDCD1, CD7, LAG3, HNRNPA1P21, FOXP3, CCR8, CXCL13, AIM2, IL2RA, ICOS, CTLA4, TNFRSF9, IL21R, BTN3A3, BTN3A1, TAP2, NLRC5, HLA-F, PSMB8, PSMB9, TAP1, HCP5, UBE2L6, PSME2, IRF1, C19orf38, IGFLR1, LINC01943, RAB33A, SLC2A6, IFI30, LILRB3, IL23A, PSME2P2, ITGAE, STAC3, FOXC1, ADAMTS9-AS2, RGN, KL, ADAMTS9-AS1, WDFY3-AS2, PTH1R, PLEKHH2, WSCD1, CABP1, CEP112, TMEM47, RCAN2, LIN7A, LEPR, PDGFA, SERTAD4-AS1, ADH1B, C7, CCL14, SELP, ACKR1, MMRN1, ITM2A, AQP1, ABI3BP, P2RY12, MPRIP, KIF13B, FYCO1, SPTLC2, ADGRA3, RBFOX2, ITGB4, KRT17, KRT16, KRT14, KRT5, DSG3, COL17A1, TMEM119, PODN, SVEP1, LAMA2, COL14A1, FGF7, OGN, PRELP, ELN, MFAP4, SSC5D, PTGDS, CHRDL1, ITGBL1, ASPN, PDGFRB, HTRA1, HEG1, ZCCHC24, SGCD, SRPX, APOD, SHC4, MIA, IL17D, LRRN4CL, BOC, PDZRN3, SFRP1, TCF7L1, CACNA1G, SPEG, COL2A1, CRISPLD1, PIANP, NACAD, EFNB3, PCYT1B, RGMA, GLI2, PCDH19, ABCA8, ADRA2A, AKAP12, ALDH3B2, APOD, ART3, ASPN, AZGP1, BLVRB, C7, CCL5, CD36, CD52, CDC20, CHI3L1, COL2A1, COL5A1, COL5A2, CRAT, CROT, CXCL10, CXCL11, CXCL13, CYP4F8, DBI, DEFB1, DHCR24, DUSP5, FABP7, FASN, FGFR4, FGL2, FOXA1, FOXC1, GABRP, GALNT7, GBP1, GCHFR, GPR87, GZMB, HGD, HTRA1, IDO1, IGFBP4, IGHM, IGJ, IL23A, IL33, INPP4B, ITM2A, JAM2, KCNK5, KIAA1324, KMO, KRT14, KRT16, KRT17, KRT6A, KRT6B, KYNU, LBP, LHFP, IGKC, MFAP4, MIA, MIDI, MYBL1, NEK2, NTN3, OGN, PI3, PLEKHB1, PMAIP1, PSMB9, PTGDS, RARRES3, RTP4, S100A1, S100A7, S100A8, SCRG1, SEMA3C, SERHL2, SFRP1, SIDT1, SOX10, SPDEF, SPRR1B, SPTLC2, SRPX, TCF7L1, TFAP2B, THBS4, TNFAIP8, TNFSF10, TRIM68, TSC22D3, UBD, UGT2B28, XBP1, ZCCHC24, and combinations thereof.

48. The method of claim 46, wherein the markers are or indicate, presence or level of a particular form of one or more genes or gene products.

49. The method of claim 47 or claim 48, wherein the candidate biomarker is selected from the group consisting of presence and level of a particular form of a gene or gene product.

50. The method of claim 49 wherein the candidate biomarker is or comprises presence or level of one or more miRNA species.

51. The method of claim 49, wherein the candidate biomarker is or comprises presence or level of one or more epigenetic modifications.

52. The method of claim 49, wherein the candidate biomarker is or comprises presence or level of one or more gene mutations.

53. The method of claim 49, wherein the candidate biomarker is or comprises presence or level of one or more gene transcript forms.

54. The method of claim 49, wherein the candidate biomarker is or comprises presence or level of one or more proteins or forms thereof.

55. A method of characterizing a potential cancer therapy by determining that it directly or indirectly correlates with an immunomodulatory (IM) status or with a subtype selected from mesenchymal (M), mesenchymal stem-like (MSL).

56. A method comprising a step of:
detecting in a subject who is a candidate for receiving a particular therapy a biomarker established to correlate with responsiveness or non-responsiveness to the therapy.

57. A method of treating a subject in whom a biomarker has been detected, the method comprising steps of:
administering immunomodulation therapy or therapy that sensitizes to immunomodulation therapy if the therapy has been correlated with IM status; and
administering alternative therapy if the biomarker has been correlated with M or MSL subtype.

58. A method of treating a subject in whom a biomarker has been detected, the method comprising steps of:
administering therapy that has been correlated with IM status if the biomarker has also been so correlated; and
administering therapy that has been correlated with M or MSL subtype if the therapy has also been so correlated.

59. The method of claim 46, wherein the candidate biomarker is or comprises a gene comprising one or more mutations.

60. A method of treating a subject, comprising steps of:
(a) assessing a biomarker indicative of immune microenvironment status; and
(b) selecting a treatment option based upon biomarker level.

61. A method of claim 60, wherein the assessment of a biomarker includes:
(a) assessing both:
(i) subtype markers of a subtype selected from mesenchymal (M), mesenchymal stem-like (MSL), and combinations thereof; and
(ii) status markers of immunomodulatory (IM) status; and
(b) calculating, by means of a computer, an IO score by weighting the subtype markers as likely to indicate non-responsiveness to immunomodulation therapy and the status markers as likely to indicate responsiveness to immunomodulation therapy.

62. The method of claim 60 or 61, wherein the biomarker is or comprises presence or level of a particular form of one or more genes or gene products.

63. The method of claim 62, wherein the one or more genes or gene products comprise one or more mutations.

64. The method of claim 62, wherein the one or more genes or gene products comprise one or more methylated genes.

65. The method of claim 64, wherein the assessing comprises assessment of methylation status and/or level.

66. The method of claim 60 or 61, wherein the biomarker is or comprises presence or level of one or more miRNA species.

67. The method of claim 66, wherein the biomarker is a miRNA present in blood.

68. The method of claim 66, wherein the biomarker is a miRNA present in a solid tumor.

69. The method of claim 60 or 61, wherein the biomarker is or comprises one or more immune cell types.

70. The method of claim 60 or 61, wherein the biomarker is or comprises one or more immune cell types.

71. The method of claim 62, wherein the gene or gene products are correlated with one or more immune cell types.

72. The method of any one of claims 60-71, wherein the treatment option comprises administration of one or more compounds.

73. The method of claim 72, wherein the subject is determined to have a tumor type sensitive to the one or more compounds.

74. The method of claim 72, wherein the subject is determined to have a tumor type with a particular IO score that may change upon administration of the one or more compounds.

75. The method of claim 74, wherein the treatment option further comprises co-administration or sequential administration of an additional or alternative therapy based upon change in IO score.

76. The method of claim 60, wherein:
the biomarker is determined to have a correlation with one or more M or MSL subtype markers; and
the biomarker is determined to have biochemical, physical, or regulatory interactions with one or more additional biomarkers determined to have a correlation with one or more IM status markers.

77. The method of claim 60, wherein:
the biomarker is determined to have a correlation with one or more IM status markers; and
the biomarker is determined to have biochemical, physical, or regulatory interactions with one or more additional biomarkers determined to have a correlation with one or more M or MSL subtype markers.
